(19) United States
(12) Patent Application Publication
(10) Pub. No.: US 2026/0101670 A1
(43) Pub. Date: Apr. 9, 2026

(54) COMPOUND, LIGHT-EMITTING DEVICE INCLUDING SAME AND ELECTRONIC APPARATUS INCLUDING SAME

(71) Applicant: Samsung Display Co., Ltd., Yongin-si (KR)

(72) Inventors: Hwasook Ryu, Yongin-si (KR); Hankyu Pak, Yongin-si (KR); Dongjun Kim, Yongin-si (KR); Chaeyeong Kim, Yongin-si (KR); Sanghyun Han, Yongin-si (KR)

(21) Appl. No.: 19/088,611

(22) Filed: Mar. 24, 2025

(30) Foreign Application Priority Data
Oct. 7, 2024 (KR) 10-2024-0136014

(51) Int. Cl. H10K 85/60; C07D 251/24; C09K 11/06; H10K 50/12; H10K 50/16; H10K 85/30; H10K 101/00

(52) U.S. Cl. CPC H10K 85/654; C07D 251/24; C09K 11/06; H10K 85/346; H10K 85/658; C09K 2211/1007; C09K 2211/1014; C09K 2211/1018; C09K 2211/1022; C09K 2211/1029; C09K 2211/1044; C09K 2211/185; H10K 50/12; H10K 50/16; H10K 2101/90

(57) ABSTRACT

Provided are a compound represented by Formula 1, a light-emitting device including the same, and an electronic device including the light-emitting device including the compound represented by Formula 1. The light-emitting device includes a first electrode, a second electrode facing the first electrode, and an interlayer between the first electrode and the second electrode and including an emission layer, wherein the interlayer includes the compound represented by Formula 1. The features of Formula 1 are as described in the present disclosure.

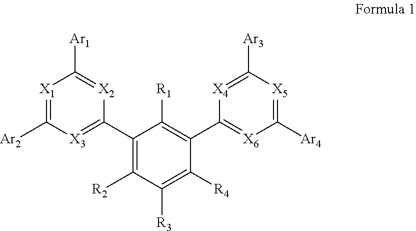

### CROSS-REFERENCE TO RELATED APPLICATION

The present application claims priority to and the benefit of Korean Patent Application No. 10-2024-0136014, filed on Oct. 7, 2024, in the Korean Intellectual Property Office, the entire content of which is hereby incorporated by reference.

### BACKGROUND

### 1. Field

One or more embodiments of the present disclosure relate to a compound, a light-emitting device including the same, and an electronic apparatus including the light-emitting device.

### 2. Description of the Related Art

From among light-emitting devices, self-emissive devices have wide viewing angles, high contrast ratios, short response times, and excellent characteristics in terms of luminance, driving voltage, and response speed.

In a light-emitting device, a first electrode is on a substrate, and a hole transport region, an emission layer, an electron transport region, and a second electrode are sequentially on the first electrode. Holes provided from the first electrode move toward the emission layer through the hole transport region, and electrons provided from the second electrode move toward the emission layer through the electron transport region. Carriers, such as holes and electrons, recombine in the emission layer to produce excitons. The excitons may transition from an excited state to a ground state, thereby generating light.

### SUMMARY

One or more embodiments of the present disclosure include a compound represented by Formula 1 and a light-emitting device including the same.

Additional aspects of embodiments will be set forth in part in the description which follows and, in part, will be apparent from the description, or may be learned by practice of the presented embodiments of the disclosure.

One or more embodiments include a compound represented by Formula 1:

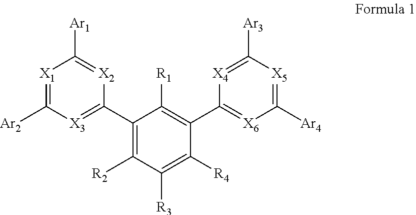

wherein, in Formula 1,
X₁ may be N or CR₁₁, X₂ may be N or CR₁₂, X₃ may be N or CR₁₃, and at least one selected from among X₁ to X₃ may be N,
X₄ may be N or CR₁₄, X₅ may be N or CR₁₅, X₆ may be N or CR₁₆, and at least one selected from among X₄ to X₆ may be N,
R₁ to R₄ and R₁₁ to R₁₆ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₃-C₁₀ cycloalkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₁-C₆₀ alkylthio group unsubstituted or substituted with at least one R₁₀ₐ, a C₆-C₆₀ aryl group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₁-C₆₀ heteroaryl group unsubstituted or substituted with at least one R₁₀ₐ, a C₆-C₆₀ non-aromatic condensed polycyclic group unsubstituted or substituted with at least one R₁₀ₐ, a C₁-C₆₀ non-aromatic condensed heteropolycyclic group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
Ar₁ to Ar₄ may each independently be a C₆-C₆₀ aryl group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₁-C₆₀ heteroaryl group unsubstituted or substituted with at least one R₁₀ₐ, a C₆-C₆₀ non-aromatic condensed polycyclic group unsubstituted or substituted with at least one R₁₀ₐ, or a C₁-C₆₀ non-aromatic condensed heteropolycyclic group unsubstituted or substituted with at least one R₁₀ₐ,
at least one selected from among Ar₁ to Ar₄ may be an unsubstituted terphenyl group, and
R₁₀ₐ may be:
hydrogen, —F, —Cl, —Br, —I, a hydroxyl group, a cyano group, or a nitro group,
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof,
a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof, or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ may each independently be hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, or a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof.

According to one or more embodiments, a light-emitting device includes a first electrode, a second electrode facing the first electrode, and an interlayer between the first electrode and the second electrode and including an emission layer, wherein the interlayer includes a compound represented by Formula 1.

According to one or more embodiments, an electronic apparatus includes the light-emitting device.

### BRIEF DESCRIPTION OF THE DRAWINGS

The above and other aspects and features of certain embodiments of the disclosure will be more apparent from the following description taken in conjunction with the accompanying drawings, in which:

FIG. 1 is a schematic drawing of the structure of a light-emitting device according to an embodiment;

FIG. 2 is a cross-sectional view of an electronic apparatus according to an embodiment; and

FIG. 3 is a cross-sectional view of an electronic apparatus according to another embodiment.

### DETAILED DESCRIPTION

Reference will now be made in more detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the drawings, to explain aspects of embodiments of the present description. As used herein, the term “and/or” includes any and all combinations of one or more of the associated listed items. Throughout the disclosure, the expression “at least one of a, b or c” indicates only a, only b, only c, both a and b, both a and c, both b and c, all of a, b, and c, or variations thereof.

While electron transport materials of the related art are reported to have high efficiency and long lifespan characteristics, further improvements in luminescence efficiency and lifespan are required or beneficial to achieve fine pitch and low power consumption for commercialization of mobile devices and large TVs using organic light-emitting devices.

According to an aspect of embodiments, a compound may be represented by Formula 1:

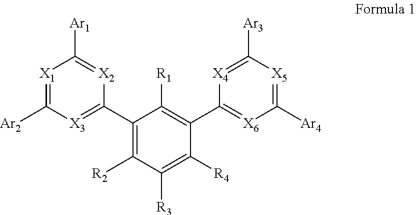

wherein, in Formula 1,
X₁ may be N or CR₁₁, X₂ may be N or CR₁₂, X₃ may be N or CR₁₃, and at least one selected from among X₁ to X₃ may be N,
X₄ may be N or CR₁₄, X₅ may be N or CR₁₅, X₆ may be N or CR₁₆, and at least one selected from among X₄ to X₆ may be N,
R₁ to R₄ and R₁₁ to R₁₆ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₃-C₁₀ cycloalkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₁-C₆₀ alkylthio group unsubstituted or substituted with at least one R₁₀ₐ, a C₆-C₆₀ aryl group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₁-C₆₀ heteroaryl group unsubstituted or substituted with at least one R₁₀ₐ, a C₆-C₆₀ non-aromatic condensed polycyclic group unsubstituted or substituted with at least one R₁₀ₐ, a C₁-C₆₀ non-aromatic condensed heteropolycyclic group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
Ar₁ to Ar₄ may each independently be a C₆-C₆₀ aryl group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₁-C₆₀ heteroaryl group unsubstituted or substituted with at least one R₁₀ₐ, a C₆-C₆₀ non-aromatic condensed polycyclic group unsubstituted or substituted with at least one R₁₀ₐ, or a C₁-C₆₀ non-aromatic condensed heteropolycyclic group unsubstituted or substituted with at least one R₁₀ₐ,
at least one selected from among Ar₁ to Ar₄ may be an unsubstituted terphenyl group,
R₁₀ₐ may be:
hydrogen, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ may each independently be hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, or a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof.

The compound represented by Formula 1 has a terphenyl group directly bonded to a triazine core, leading to excellent charge transport capability. A light-emitting device including the compound of Formula 1 may have excellent driving voltage, efficiency, and lifespan.

According to an embodiment, the unsubstituted terphenyl group may be represented by Formula Y1 or Formula Y2:

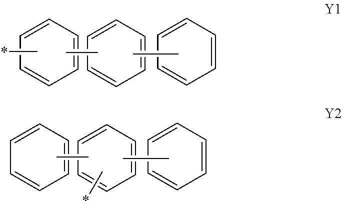

wherein * in Formulae Y1 and Y2 indicates a binding site to a neighboring atom.

According to an embodiment, the unsubstituted terphenyl group may be any one selected from among Formulae Y11 to Y25:

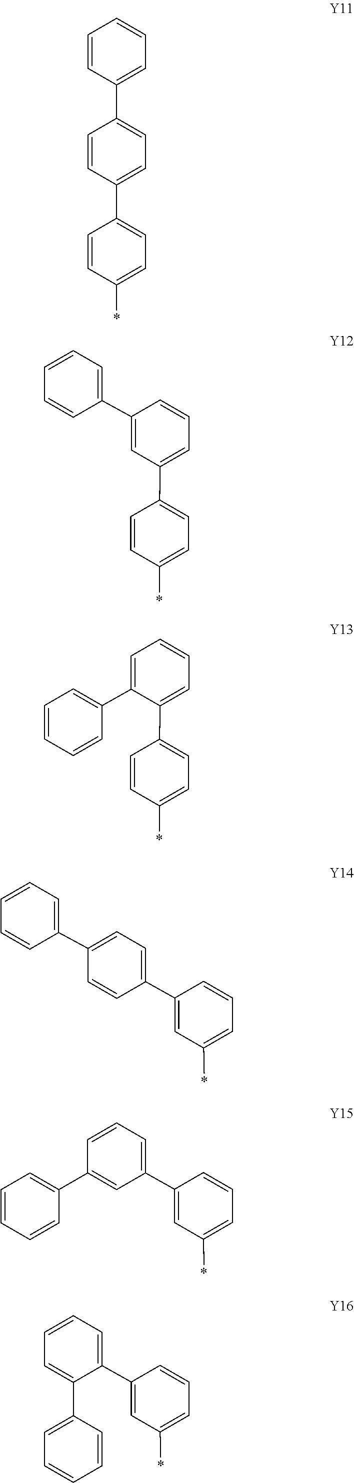

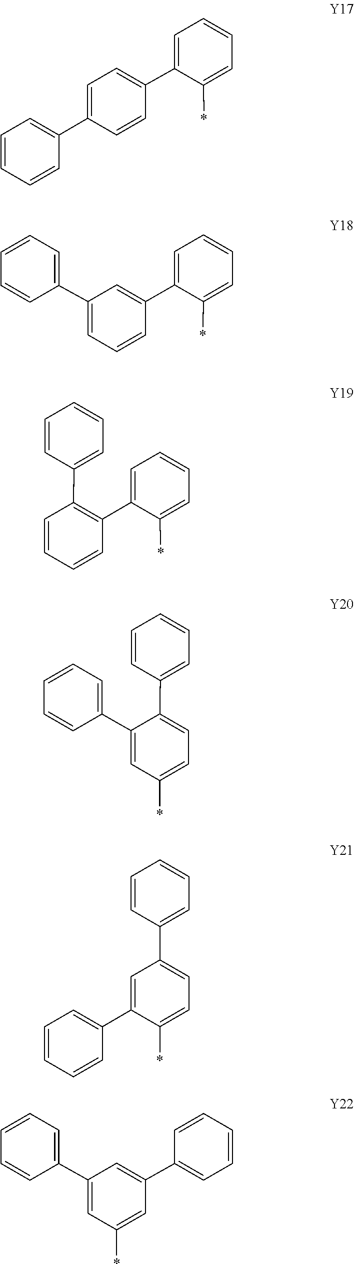

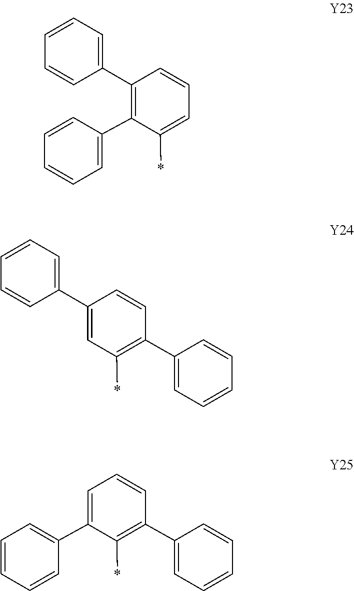 [SMILES: *c1cccc(-c2ccccc2)c1-c1ccccc1 ; *c1cc(-c2ccccc2)ccc1-c1ccccc1 ; *c1c(-c2ccccc2)cccc1-c1ccccc1]

wherein * in Formulae Y11 to Y25 indicates a binding site to a neighboring atom.

According to an embodiment, the substituents other than the unsubstituted terphenyl groups of Ar₁ to Ar₄ may each independently be a C₆-C₆₀ aryl group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, or a C₁-C₆₀ heteroaryl group unsubstituted or substituted with at least one R₁₀ₐ. For example, the substituents other than the unsubstituted terphenyl groups of Ar₁ to Ar₄ may each independently be a C₆-C₆₀ aryl group unsubstituted or substituted with at least one R₁₀ₐ.

According to an embodiment, Ar₁ or Ar₂ may be an unsubstituted terphenyl group.

According to an embodiment, Ar₃ or Ar₄ may be an unsubstituted terphenyl group.

According to an embodiment, Ar₁ or Ar₃ may be an unsubstituted terphenyl group.

According to an embodiment, R₁ to R₄ may each independently be —F, —Cl, —Br, —I, a cyano group, or a C₁-C₆₀ alkyl group unsubstituted or substituted with at least one R₁₀ₐ.

According to an embodiment, R₁₁ and R₁₅ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₃-C₁₀ cycloalkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₁-C₆₀ alkylthio group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₁-C₆₀ heteroaryl group unsubstituted or substituted with at least one R₁₀ₐ, a C₆-C₆₀ non-aromatic condensed polycyclic group unsubstituted or substituted with at least one R₁₀ₐ, a C₁-C₆₀ non-aromatic condensed heteropolycyclic group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂).

Q₁, Q₂ and Q₃ are as described above.

In an embodiment, the compound represented by Formula 1 may include one selected from among the following compounds:

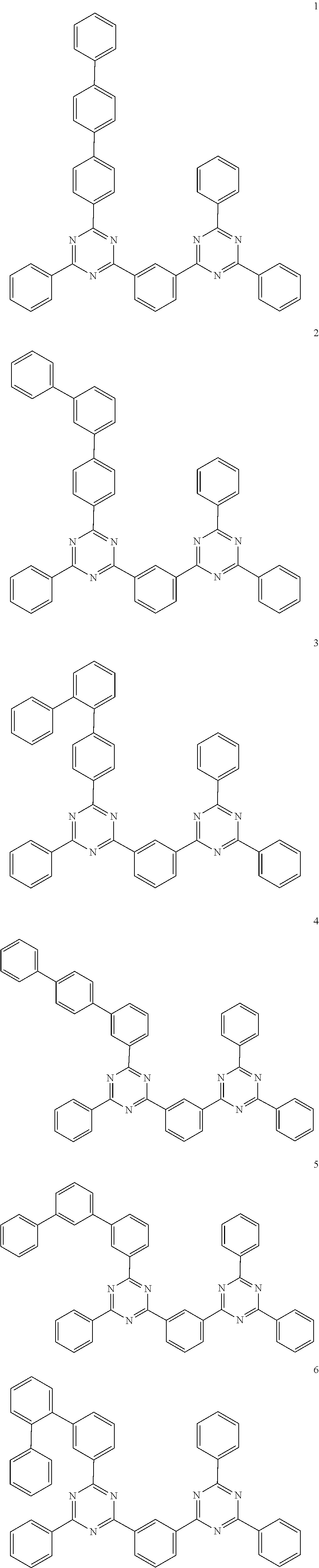

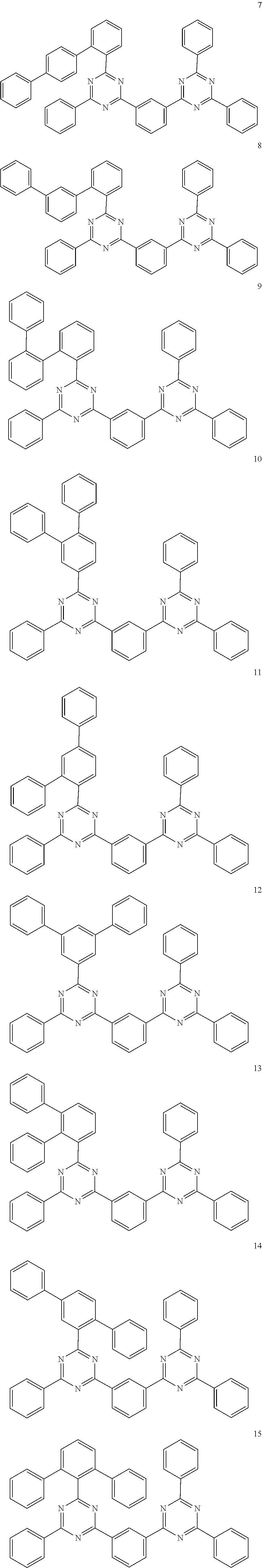

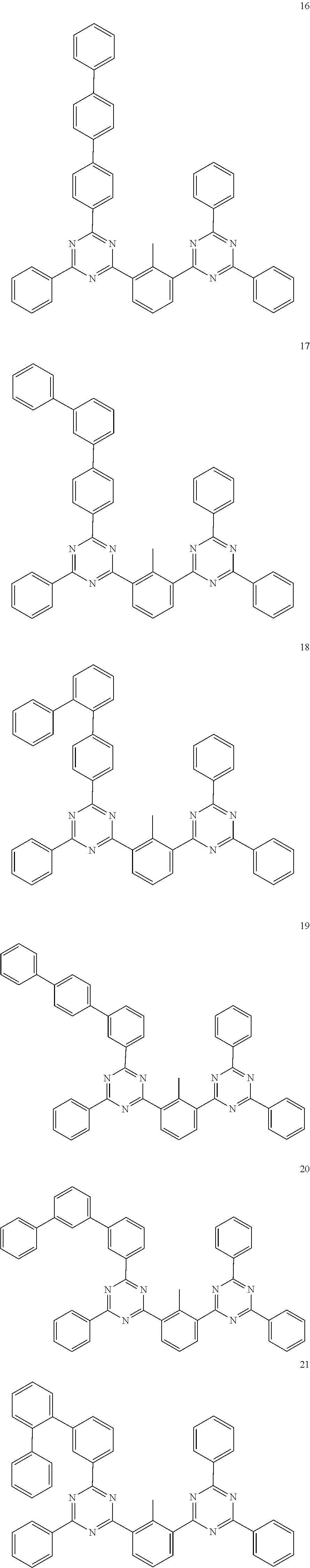

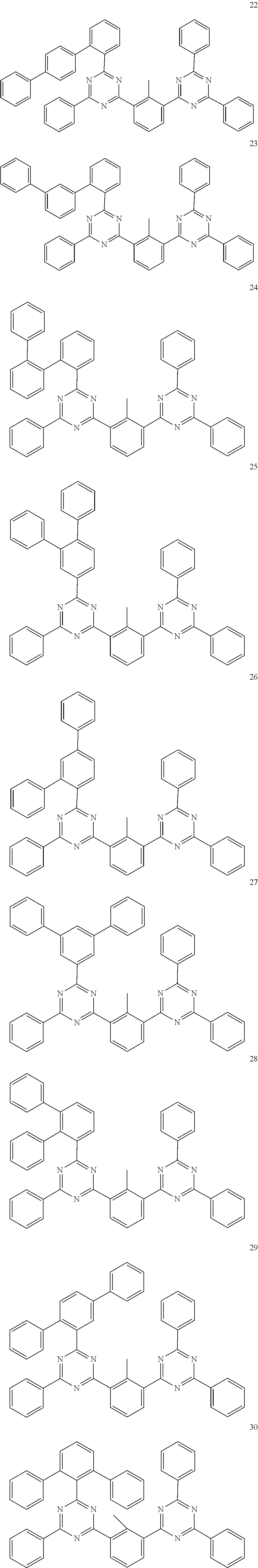

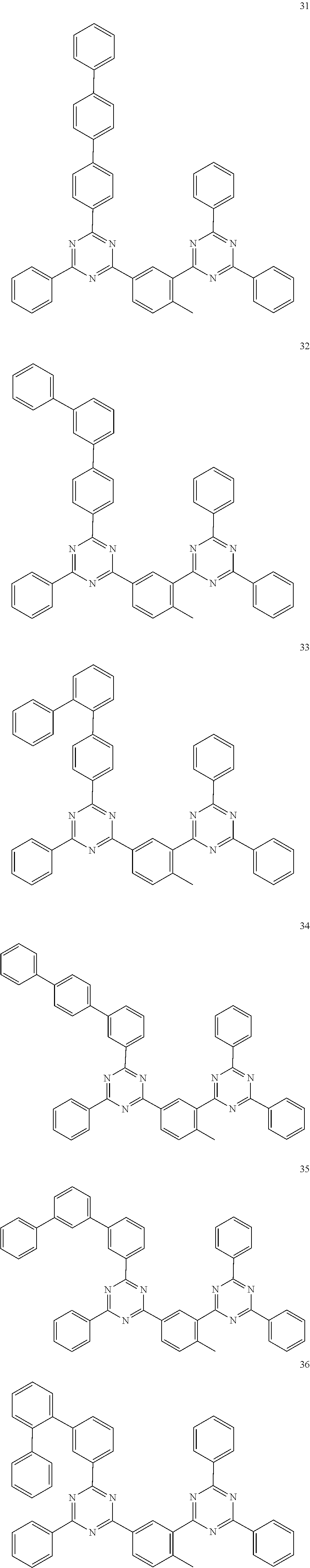

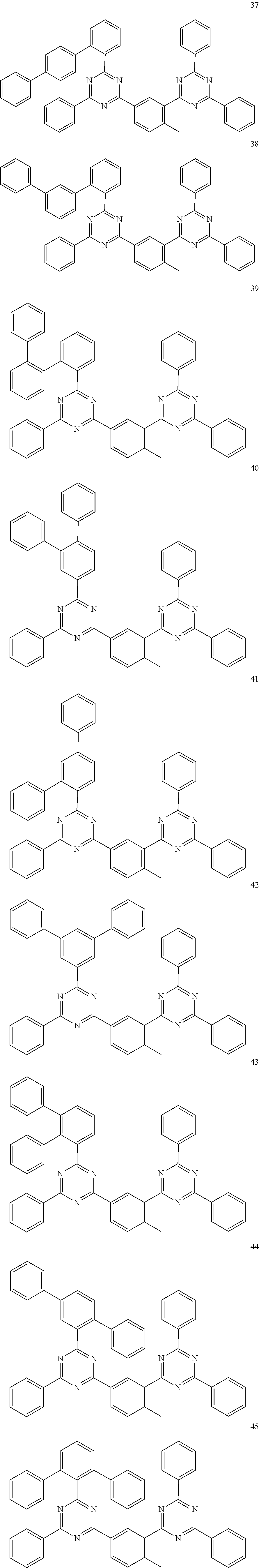

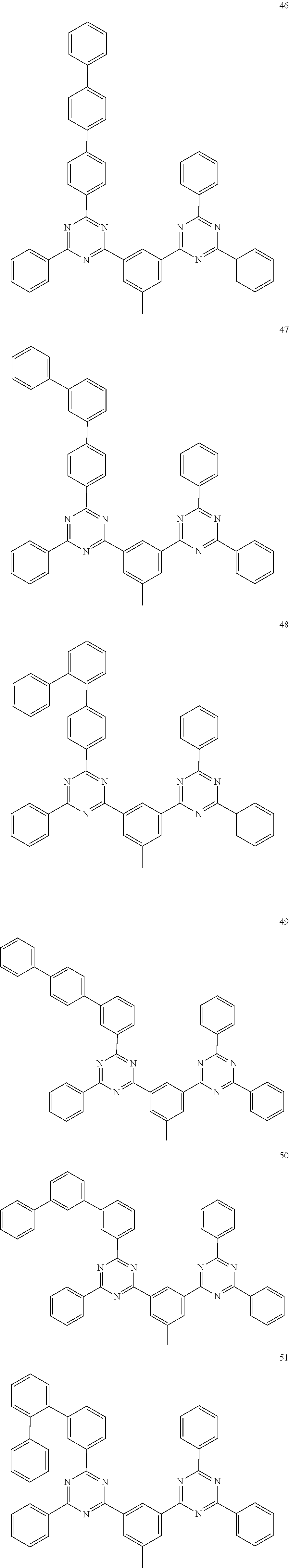

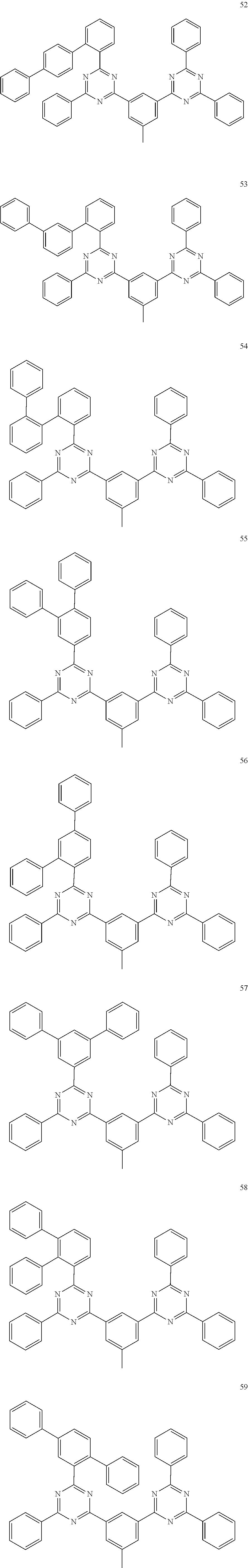

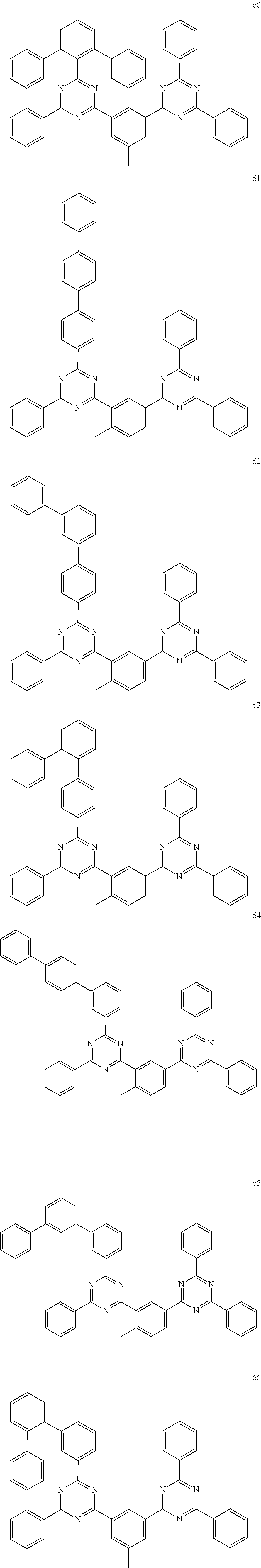

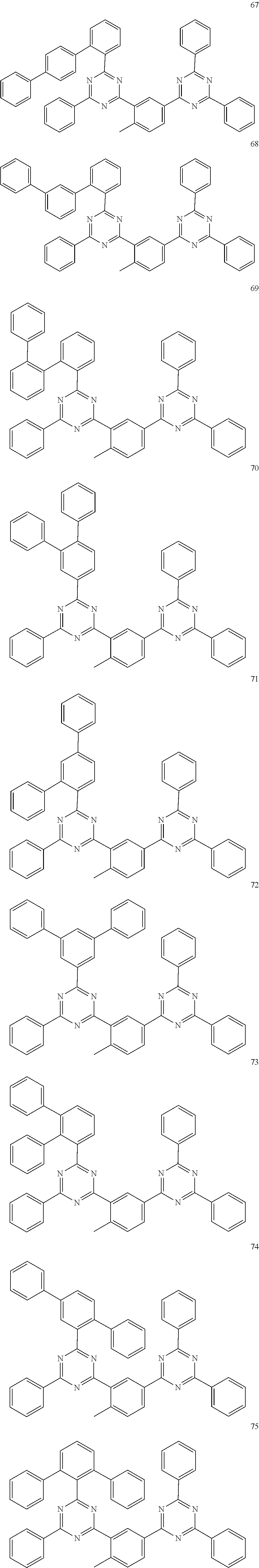

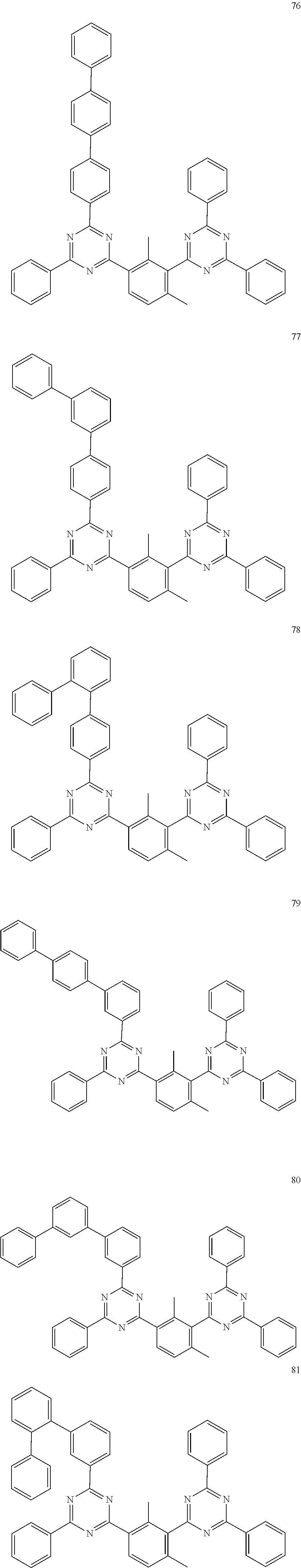

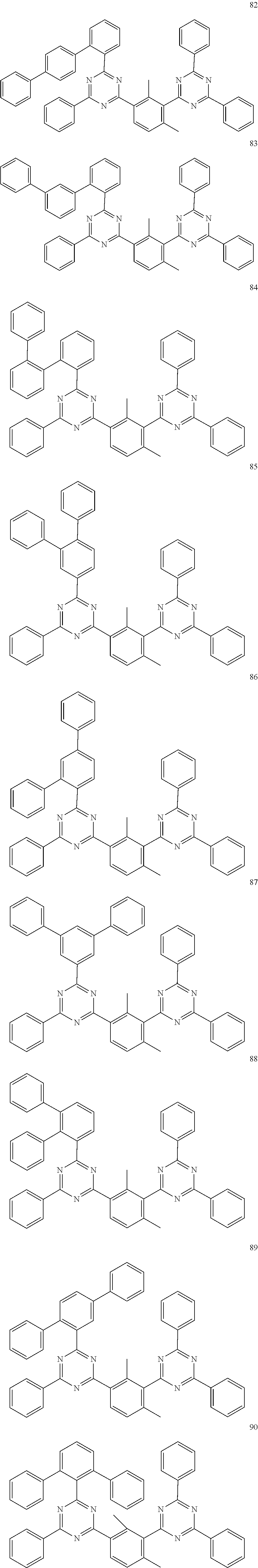

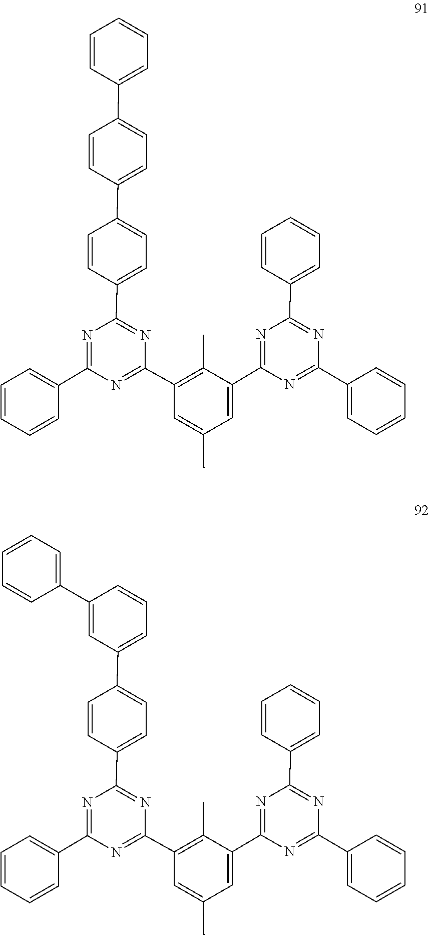 [SMILES: Cc1cc(-c2nc(-c3ccccc3)nc(-c3ccccc3)n2)c(C)c(-c2nc(-c3ccccc3)nc(-c3ccc(-c4ccc(-c5ccccc5)cc4)cc3)n2)c1 ; Cc1cc(-c2nc(-c3ccccc3)nc(-c3ccccc3)n2)c(C)c(-c2nc(-c3ccccc3)nc(-c3ccc(-c4cccc(-c5ccccc5)c4)cc3)n2)c1]

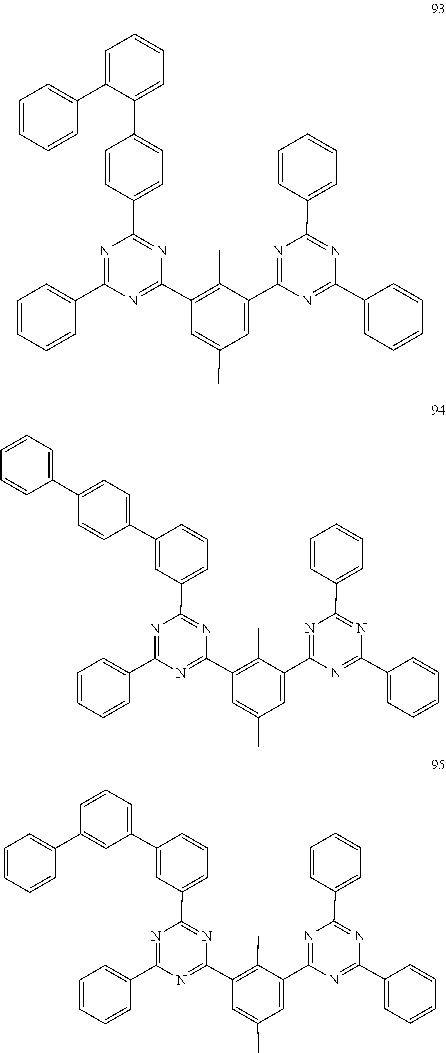 [SMILES: Cc1cc(-c2nc(-c3ccccc3)nc(-c3ccccc3)n2)c(C)c(-c2nc(-c3ccccc3)nc(-c3ccc(-c4ccccc4-c4ccccc4)cc3)n2)c1 ; Cc1cc(-c2nc(-c3ccccc3)nc(-c3ccccc3)n2)c(C)c(-c2nc(-c3ccccc3)nc(-c3cccc(-c4ccc(-c5ccccc5)cc4)c3)n2)c1 ; Cc1cc(-c2nc(-c3ccccc3)nc(-c3ccccc3)n2)c(C)c(-c2nc(-c3ccccc3)nc(-c3cccc(-c4cccc(-c5ccccc5)c4)c3)n2)c1]

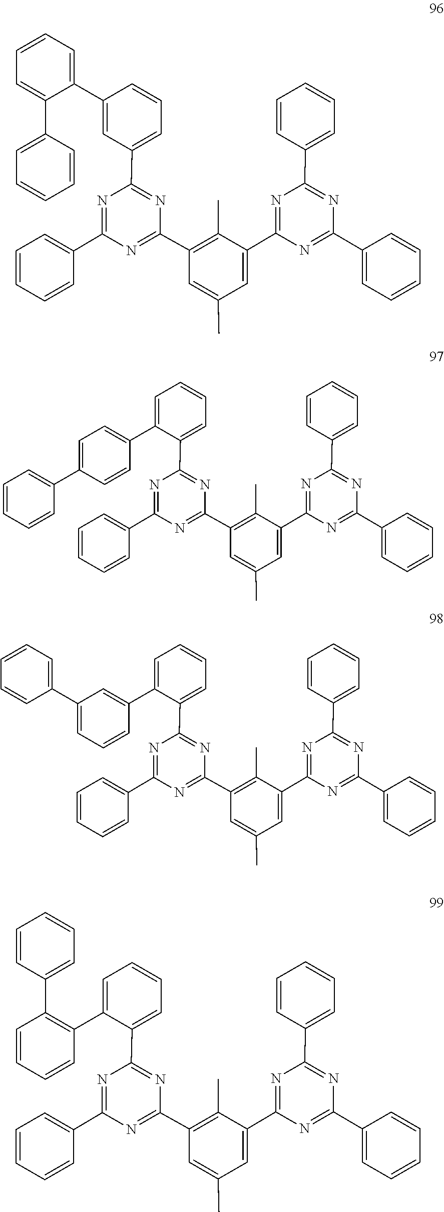 [SMILES: Cc1cc(-c2nc(-c3ccccc3)nc(-c3ccccc3)n2)c(C)c(-c2nc(-c3ccccc3)nc(-c3cccc(-c4ccccc4-c4ccccc4)c3)n2)c1 ; Cc1cc(-c2nc(-c3ccccc3)nc(-c3ccccc3)n2)c(C)c(-c2nc(-c3ccccc3)nc(-c3ccccc3-c3ccc(-c4ccccc4)cc3)n2)c1 ; Cc1cc(-c2nc(-c3ccccc3)nc(-c3ccccc3)n2)c(C)c(-c2nc(-c3ccccc3)nc(-c3ccccc3-c3cccc(-c4ccccc4)c3)n2)c1 ; Cc1cc(-c2nc(-c3ccccc3)nc(-c3ccccc3)n2)c(C)c(-c2nc(-c3ccccc3)nc(-c3ccccc3-c3ccccc3-c3ccccc3)n2)c1]

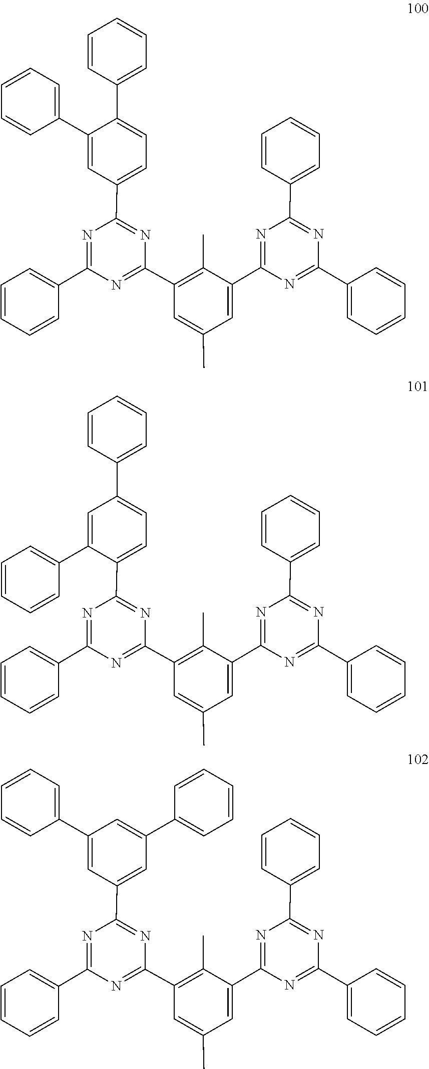

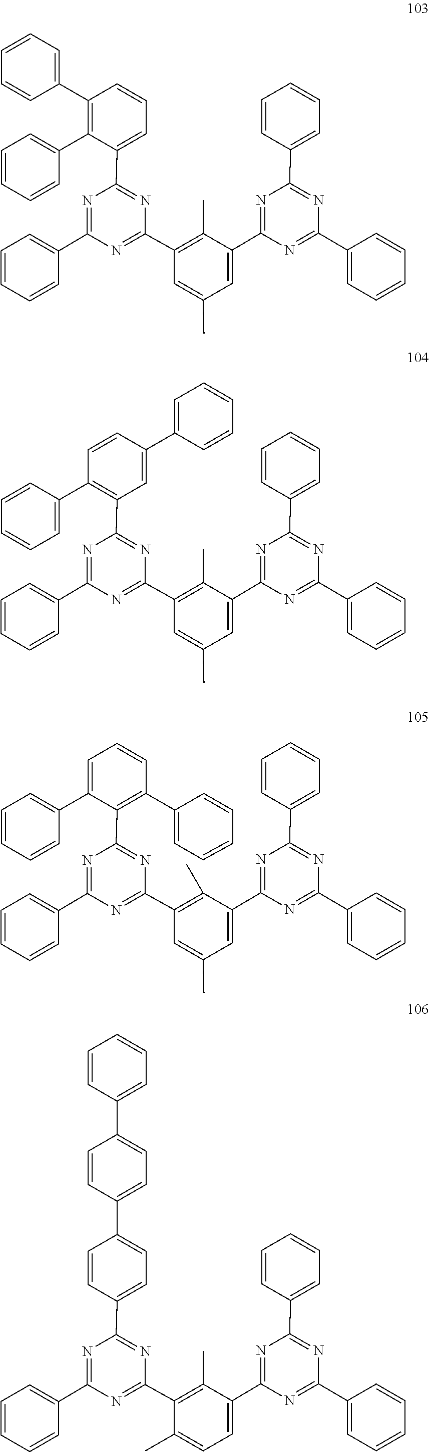 [SMILES: Cc1cc(-c2nc(-c3ccccc3)nc(-c3ccccc3)n2)c(C)c(-c2nc(-c3ccccc3)nc(-c3cccc(-c4ccccc4)c3-c3ccccc3)n2)c1 ; Cc1cc(-c2nc(-c3ccccc3)nc(-c3ccccc3)n2)c(C)c(-c2nc(-c3ccccc3)nc(-c3cc(-c4ccccc4)ccc3-c3ccccc3)n2)c1 ; Cc1cc(-c2nc(-c3ccccc3)nc(-c3ccccc3)n2)c(C)c(-c2nc(-c3ccccc3)nc(-c3c(-c4ccccc4)cccc3-c3ccccc3)n2)c1 ; Cc1ccc(-c2nc(-c3ccccc3)nc(-c3ccccc3)n2)c(C)c1-c1nc(-c2ccccc2)nc(-c2ccc(-c3ccc(-c4ccccc4)cc3)cc2)n1]

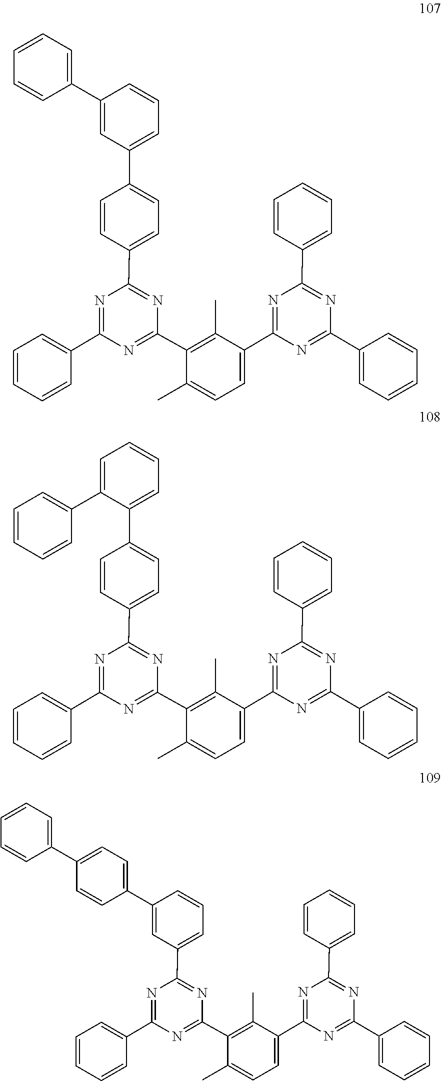

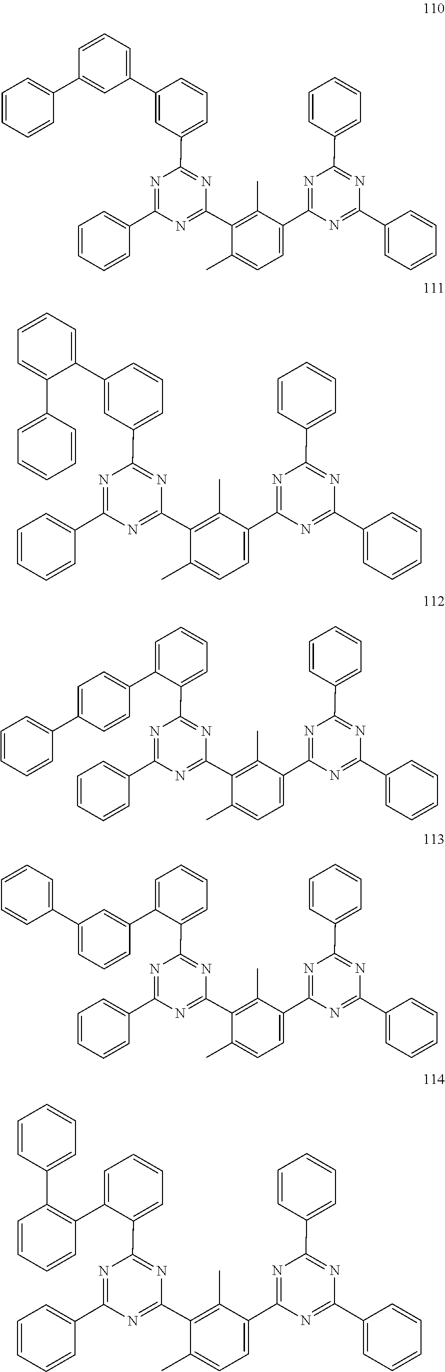 [SMILES: Cc1ccc(-c2nc(-c3ccccc3)nc(-c3ccccc3)n2)c(C)c1-c1nc(-c2ccccc2)nc(-c2cccc(-c3cccc(-c4ccccc4)c3)c2)n1 ; Cc1ccc(-c2nc(-c3ccccc3)nc(-c3ccccc3)n2)c(C)c1-c1nc(-c2ccccc2)nc(-c2cccc(-c3ccccc3-c3ccccc3)c2)n1 ; Cc1ccc(-c2nc(-c3ccccc3)nc(-c3ccccc3)n2)c(C)c1-c1nc(-c2ccccc2)nc(-c2ccccc2-c2ccc(-c3ccccc3)cc2)n1 ; Cc1ccc(-c2nc(-c3ccccc3)nc(-c3ccccc3)n2)c(C)c1-c1nc(-c2ccccc2)nc(-c2ccccc2-c2cccc(-c3ccccc3)c2)n1 ; Cc1ccc(-c2nc(-c3ccccc3)nc(-c3ccccc3)n2)c(C)c1-c1nc(-c2ccccc2)nc(-c2ccccc2-c2ccccc2-c2ccccc2)n1]

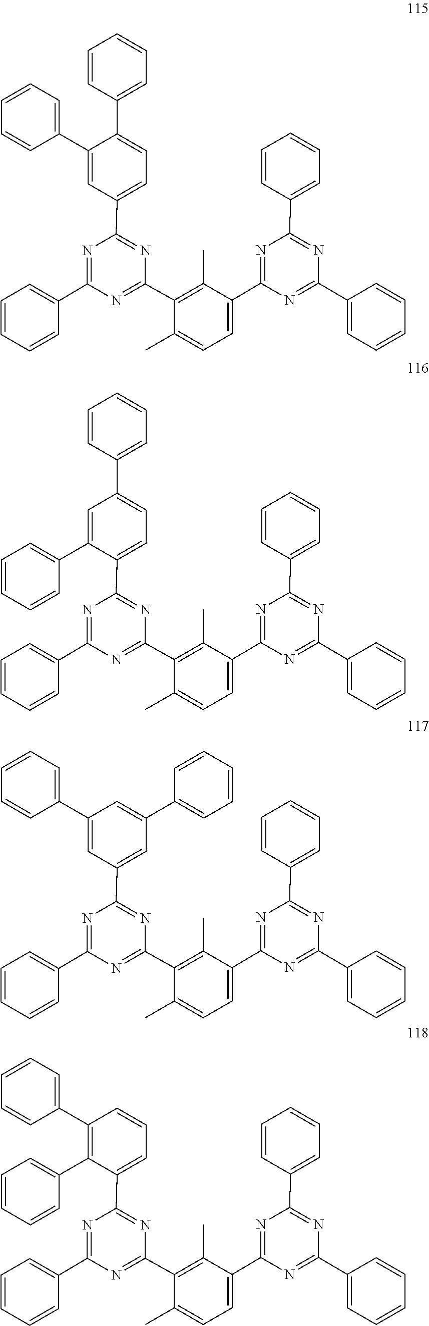

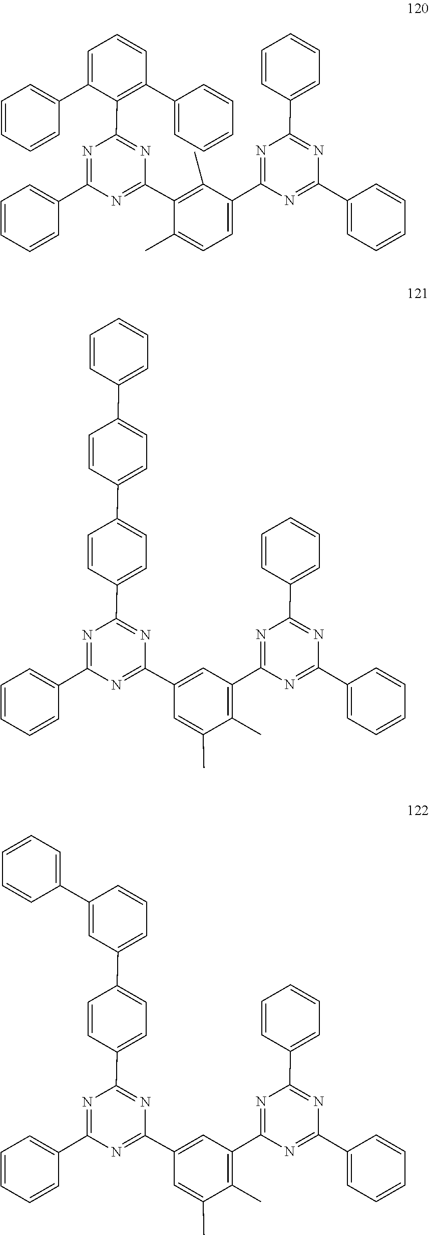 [SMILES: Cc1ccc(-c2nc(-c3ccccc3)nc(-c3ccccc3)n2)c(C)c1-c1nc(-c2ccccc2)nc(-c2c(-c3ccccc3)cccc2-c2ccccc2)n1 ; Cc1cc(-c2nc(-c3ccccc3)nc(-c3ccc(-c4ccc(-c5ccccc5)cc4)cc3)n2)cc(-c2nc(-c3ccccc3)nc(-c3ccccc3)n2)c1C ; Cc1cc(-c2nc(-c3ccccc3)nc(-c3ccc(-c4cccc(-c5ccccc5)c4)cc3)n2)cc(-c2nc(-c3ccccc3)nc(-c3ccccc3)n2)c1C]

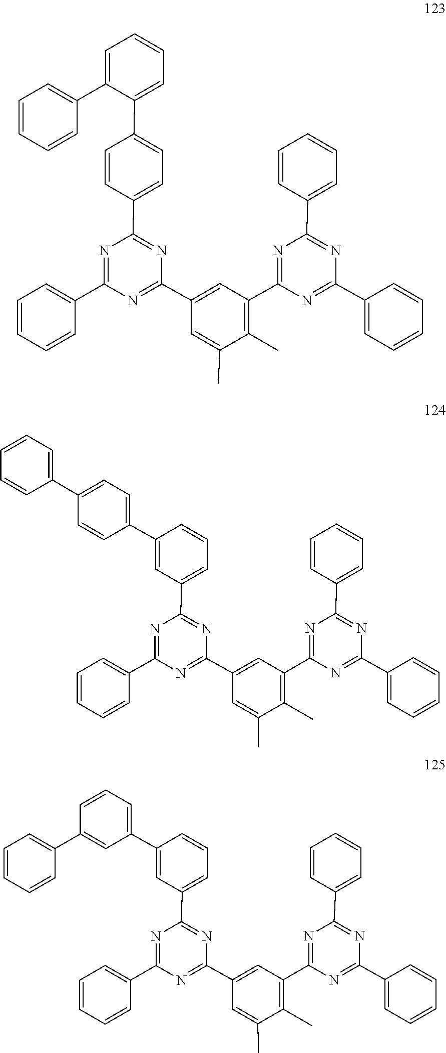 [SMILES: Cc1cc(-c2nc(-c3ccccc3)nc(-c3ccc(-c4ccccc4-c4ccccc4)cc3)n2)cc(-c2nc(-c3ccccc3)nc(-c3ccccc3)n2)c1C ; Cc1cc(-c2nc(-c3ccccc3)nc(-c3cccc(-c4ccc(-c5ccccc5)cc4)c3)n2)cc(-c2nc(-c3ccccc3)nc(-c3ccccc3)n2)c1C ; Cc1cc(-c2nc(-c3ccccc3)nc(-c3cccc(-c4cccc(-c5ccccc5)c4)c3)n2)cc(-c2nc(-c3ccccc3)nc(-c3ccccc3)n2)c1C]

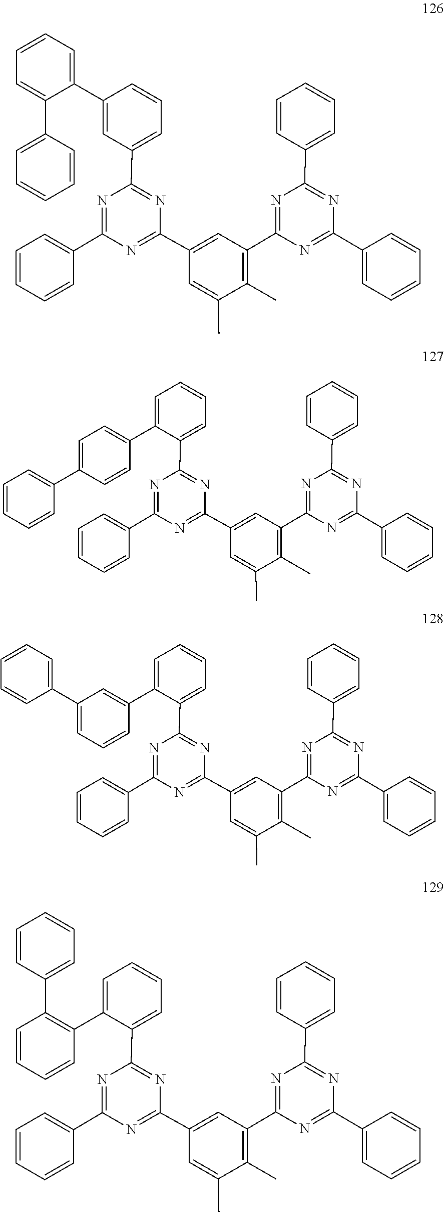 [SMILES: Cc1cc(-c2nc(-c3ccccc3)nc(-c3cccc(-c4ccccc4-c4ccccc4)c3)n2)cc(-c2nc(-c3ccccc3)nc(-c3ccccc3)n2)c1C ; Cc1cc(-c2nc(-c3ccccc3)nc(-c3ccccc3-c3ccc(-c4ccccc4)cc3)n2)cc(-c2nc(-c3ccccc3)nc(-c3ccccc3)n2)c1C ; Cc1cc(-c2nc(-c3ccccc3)nc(-c3ccccc3-c3cccc(-c4ccccc4)c3)n2)cc(-c2nc(-c3ccccc3)nc(-c3ccccc3)n2)c1C ; Cc1cc(-c2nc(-c3ccccc3)nc(-c3ccccc3-c3ccccc3-c3ccccc3)n2)cc(-c2nc(-c3ccccc3)nc(-c3ccccc3)n2)c1C]

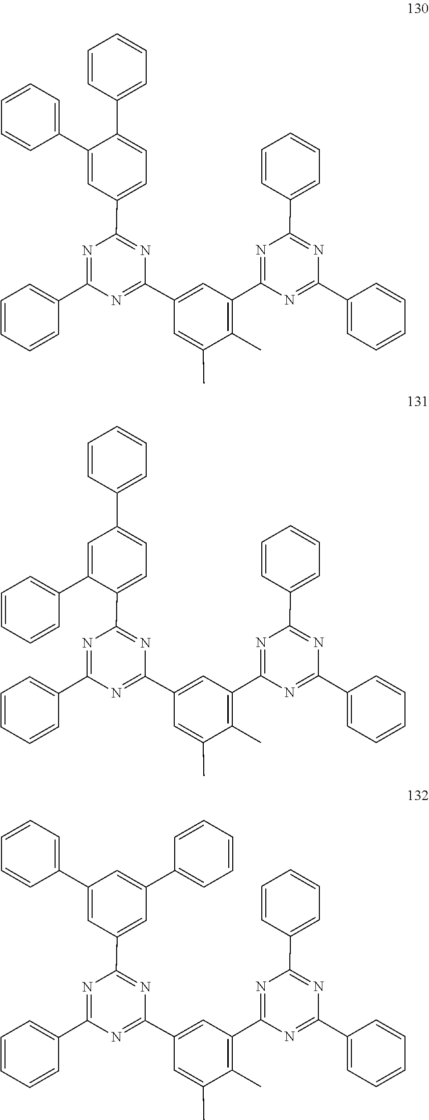

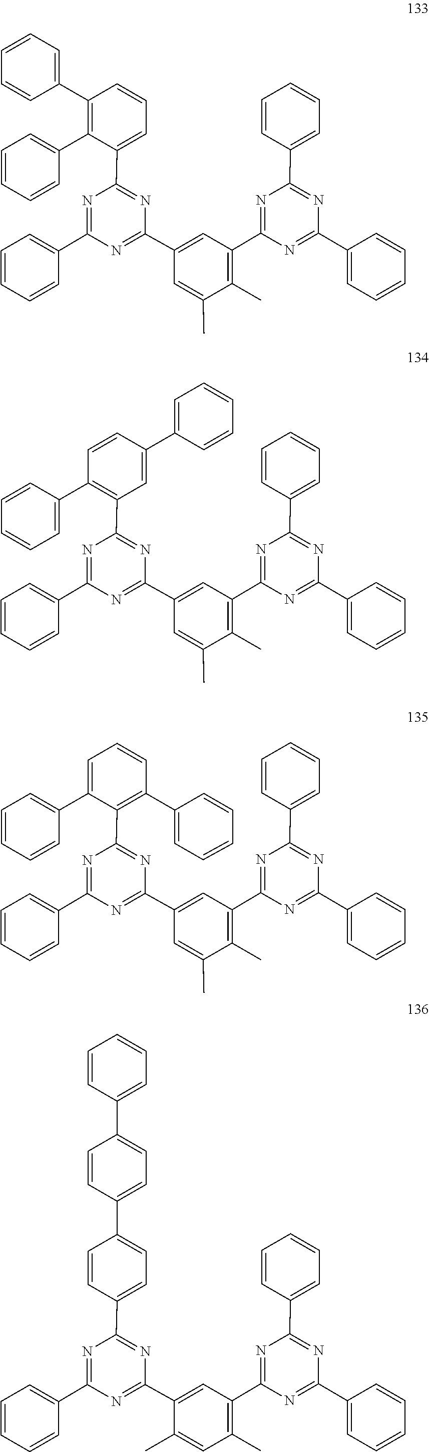 [SMILES: Cc1cc(-c2nc(-c3ccccc3)nc(-c3cccc(-c4ccccc4)c3-c3ccccc3)n2)cc(-c2nc(-c3ccccc3)nc(-c3ccccc3)n2)c1C ; Cc1cc(-c2nc(-c3ccccc3)nc(-c3cc(-c4ccccc4)ccc3-c3ccccc3)n2)cc(-c2nc(-c3ccccc3)nc(-c3ccccc3)n2)c1C ; Cc1cc(-c2nc(-c3ccccc3)nc(-c3c(-c4ccccc4)cccc3-c3ccccc3)n2)cc(-c2nc(-c3ccccc3)nc(-c3ccccc3)n2)c1C ; Cc1cc(C)c(-c2nc(-c3ccccc3)nc(-c3ccc(-c4ccc(-c5ccccc5)cc4)cc3)n2)cc1-c1nc(-c2ccccc2)nc(-c2ccccc2)n1]

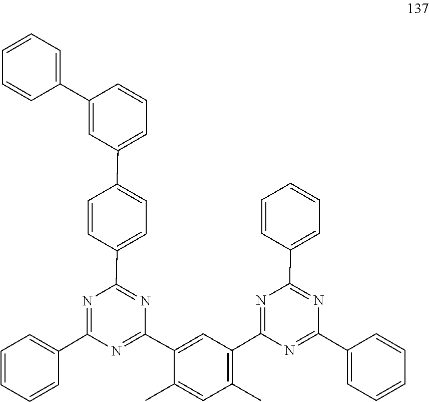 [SMILES: Cc1cc(C)c(-c2nc(-c3ccccc3)nc(-c3ccc(-c4cccc(-c5ccccc5)c4)cc3)n2)cc1-c1nc(-c2ccccc2)nc(-c2ccccc2)n1]

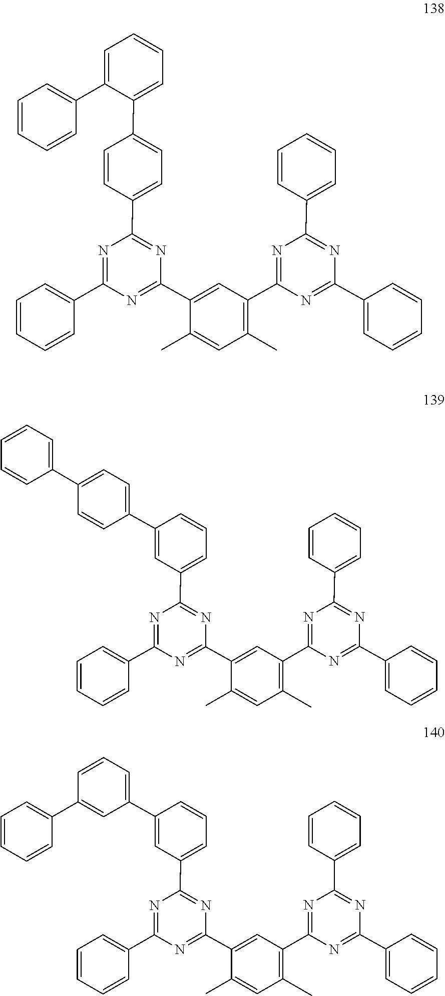 [SMILES: Cc1cc(C)c(-c2nc(-c3ccccc3)nc(-c3ccc(-c4ccccc4-c4ccccc4)cc3)n2)cc1-c1nc(-c2ccccc2)nc(-c2ccccc2)n1 ; Cc1cc(C)c(-c2nc(-c3ccccc3)nc(-c3cccc(-c4ccc(-c5ccccc5)cc4)c3)n2)cc1-c1nc(-c2ccccc2)nc(-c2ccccc2)n1 ; Cc1cc(C)c(-c2nc(-c3ccccc3)nc(-c3cccc(-c4cccc(-c5ccccc5)c4)c3)n2)cc1-c1nc(-c2ccccc2)nc(-c2ccccc2)n1]

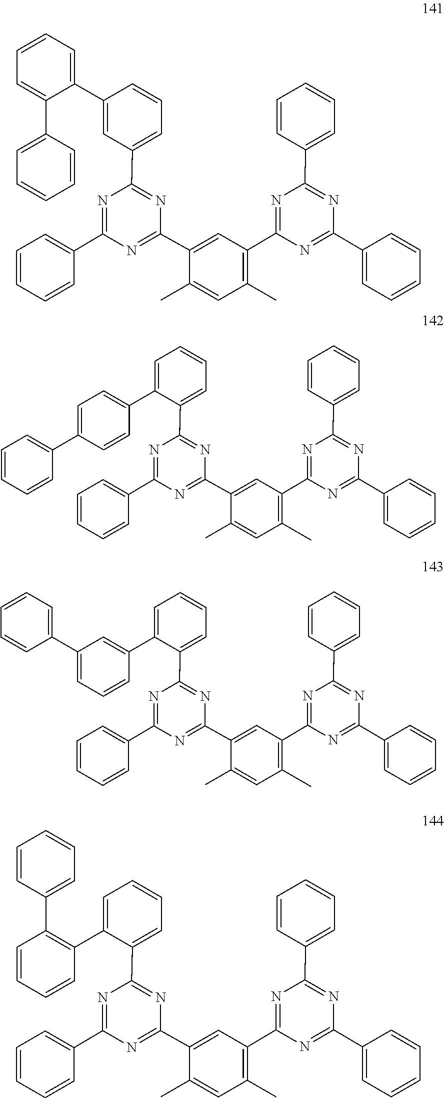 [SMILES: Cc1cc(C)c(-c2nc(-c3ccccc3)nc(-c3cccc(-c4ccccc4-c4ccccc4)c3)n2)cc1-c1nc(-c2ccccc2)nc(-c2ccccc2)n1 ; Cc1cc(C)c(-c2nc(-c3ccccc3)nc(-c3ccccc3-c3ccc(-c4ccccc4)cc3)n2)cc1-c1nc(-c2ccccc2)nc(-c2ccccc2)n1 ; Cc1cc(C)c(-c2nc(-c3ccccc3)nc(-c3ccccc3-c3cccc(-c4ccccc4)c3)n2)cc1-c1nc(-c2ccccc2)nc(-c2ccccc2)n1 ; Cc1cc(C)c(-c2nc(-c3ccccc3)nc(-c3ccccc3-c3ccccc3-c3ccccc3)n2)cc1-c1nc(-c2ccccc2)nc(-c2ccccc2)n1]

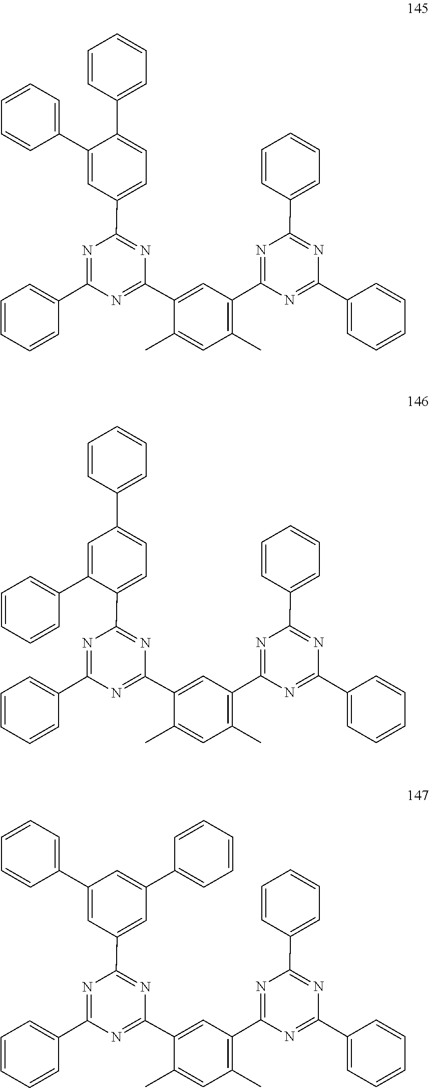 [SMILES: Cc1cc(C)c(-c2nc(-c3ccccc3)nc(-c3ccc(-c4ccccc4)c(-c4ccccc4)c3)n2)cc1-c1nc(-c2ccccc2)nc(-c2ccccc2)n1 ; Cc1cc(C)c(-c2nc(-c3ccccc3)nc(-c3ccc(-c4ccccc4)cc3-c3ccccc3)n2)cc1-c1nc(-c2ccccc2)nc(-c2ccccc2)n1 ; Cc1cc(C)c(-c2nc(-c3ccccc3)nc(-c3cc(-c4ccccc4)cc(-c4ccccc4)c3)n2)cc1-c1nc(-c2ccccc2)nc(-c2ccccc2)n1]

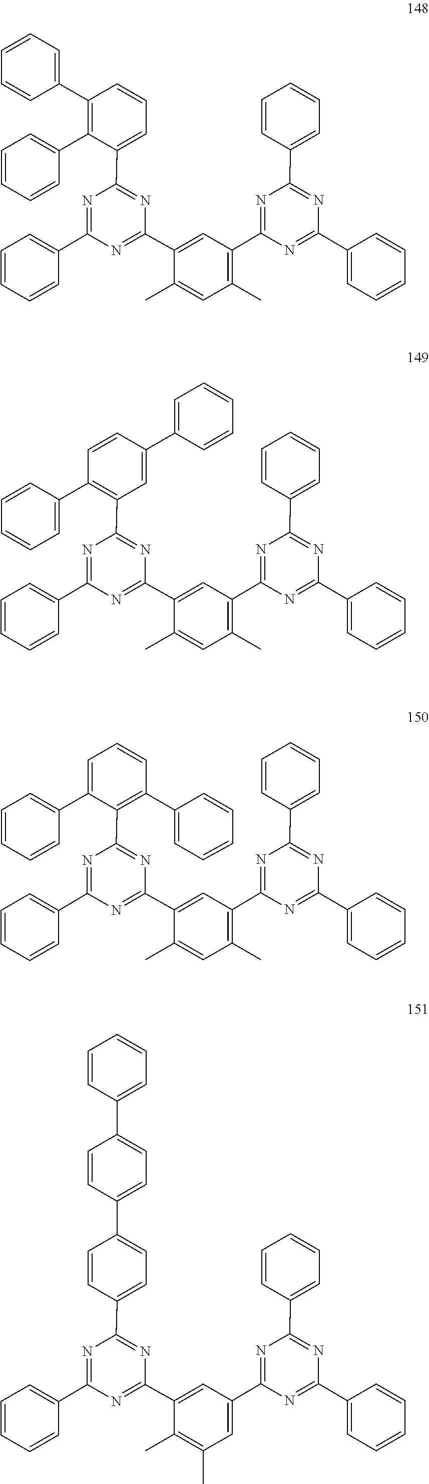 [SMILES: Cc1cc(C)c(-c2nc(-c3ccccc3)nc(-c3cccc(-c4ccccc4)c3-c3ccccc3)n2)cc1-c1nc(-c2ccccc2)nc(-c2ccccc2)n1 ; Cc1cc(C)c(-c2nc(-c3ccccc3)nc(-c3cc(-c4ccccc4)ccc3-c3ccccc3)n2)cc1-c1nc(-c2ccccc2)nc(-c2ccccc2)n1 ; Cc1cc(C)c(-c2nc(-c3ccccc3)nc(-c3c(-c4ccccc4)cccc3-c3ccccc3)n2)cc1-c1nc(-c2ccccc2)nc(-c2ccccc2)n1 ; Cc1cc(-c2nc(-c3ccccc3)nc(-c3ccccc3)n2)cc(-c2nc(-c3ccccc3)nc(-c3ccc(-c4ccc(-c5ccccc5)cc4)cc3)n2)c1C]

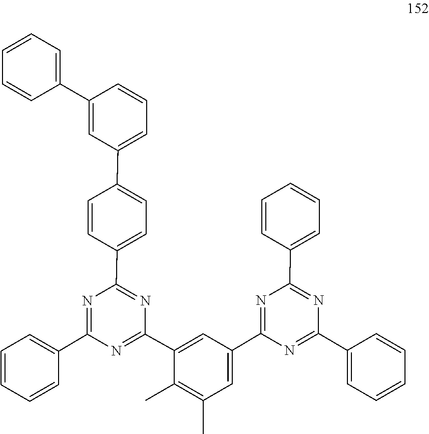 [SMILES: Cc1cc(-c2nc(-c3ccccc3)nc(-c3ccccc3)n2)cc(-c2nc(-c3ccccc3)nc(-c3ccc(-c4cccc(-c5ccccc5)c4)cc3)n2)c1C]

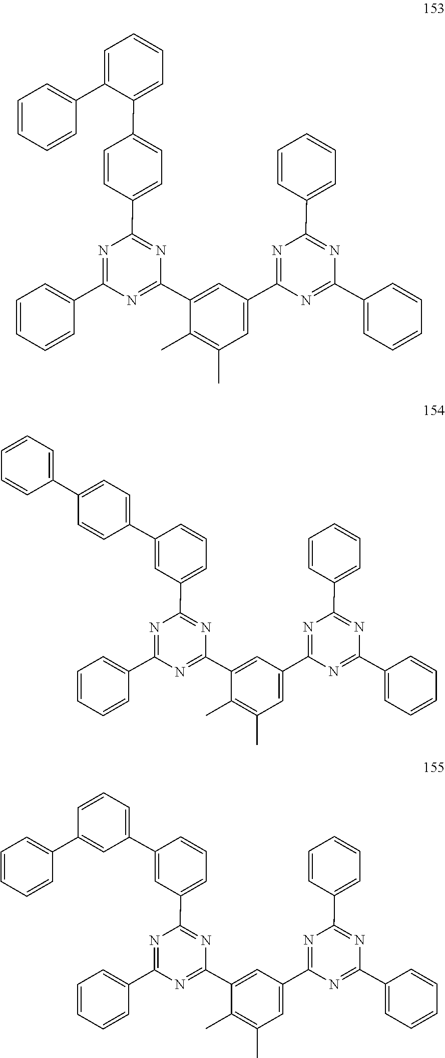 [SMILES: Cc1cc(-c2nc(-c3ccccc3)nc(-c3ccccc3)n2)cc(-c2nc(-c3ccccc3)nc(-c3ccc(-c4ccccc4-c4ccccc4)cc3)n2)c1C ; Cc1cc(-c2nc(-c3ccccc3)nc(-c3ccccc3)n2)cc(-c2nc(-c3ccccc3)nc(-c3cccc(-c4ccc(-c5ccccc5)cc4)c3)n2)c1C ; Cc1cc(-c2nc(-c3ccccc3)nc(-c3ccccc3)n2)cc(-c2nc(-c3ccccc3)nc(-c3cccc(-c4cccc(-c5ccccc5)c4)c3)n2)c1C]

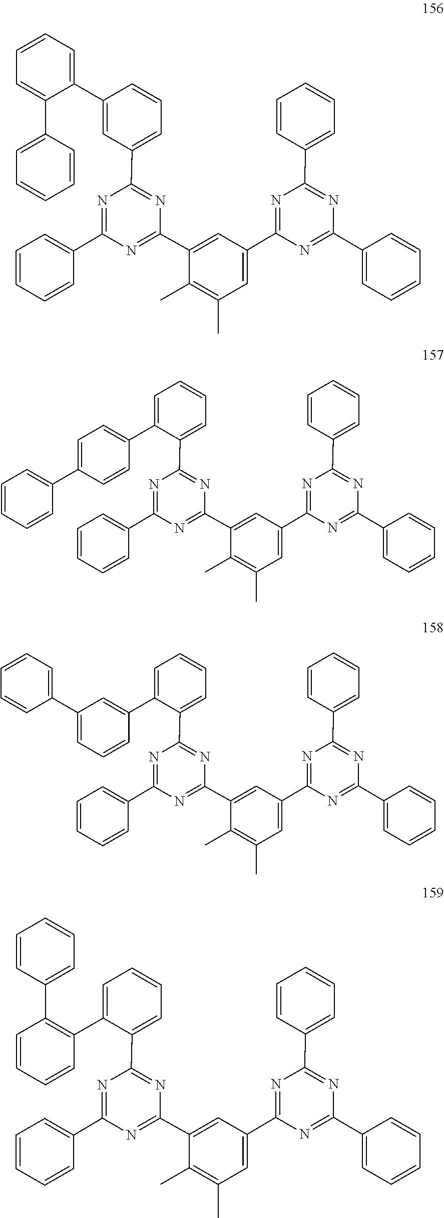 [SMILES: Cc1cc(-c2nc(-c3ccccc3)nc(-c3ccccc3)n2)cc(-c2nc(-c3ccccc3)nc(-c3cccc(-c4ccccc4-c4ccccc4)c3)n2)c1C ; Cc1cc(-c2nc(-c3ccccc3)nc(-c3ccccc3)n2)cc(-c2nc(-c3ccccc3)nc(-c3ccccc3-c3ccc(-c4ccccc4)cc3)n2)c1C ; Cc1cc(-c2nc(-c3ccccc3)nc(-c3ccccc3)n2)cc(-c2nc(-c3ccccc3)nc(-c3ccccc3-c3cccc(-c4ccccc4)c3)n2)c1C ; Cc1cc(-c2nc(-c3ccccc3)nc(-c3ccccc3)n2)cc(-c2nc(-c3ccccc3)nc(-c3ccccc3-c3ccccc3-c3ccccc3)n2)c1C]

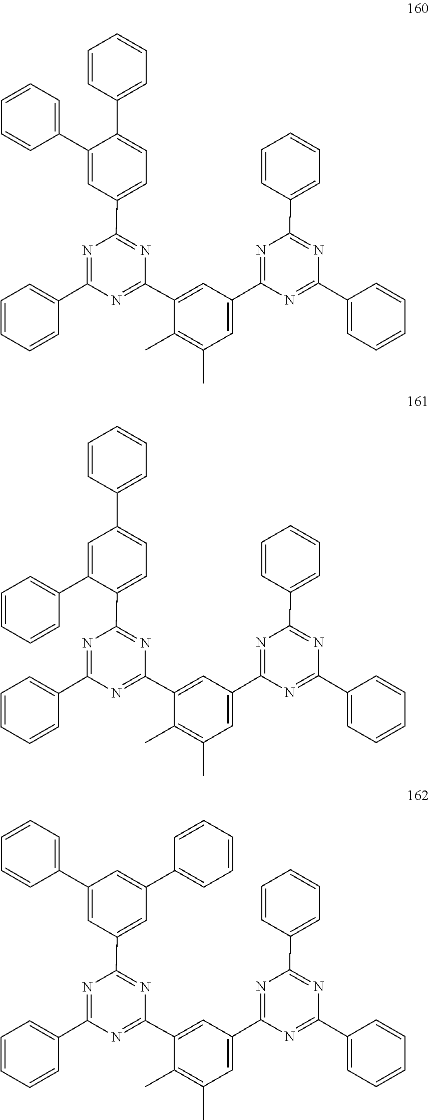

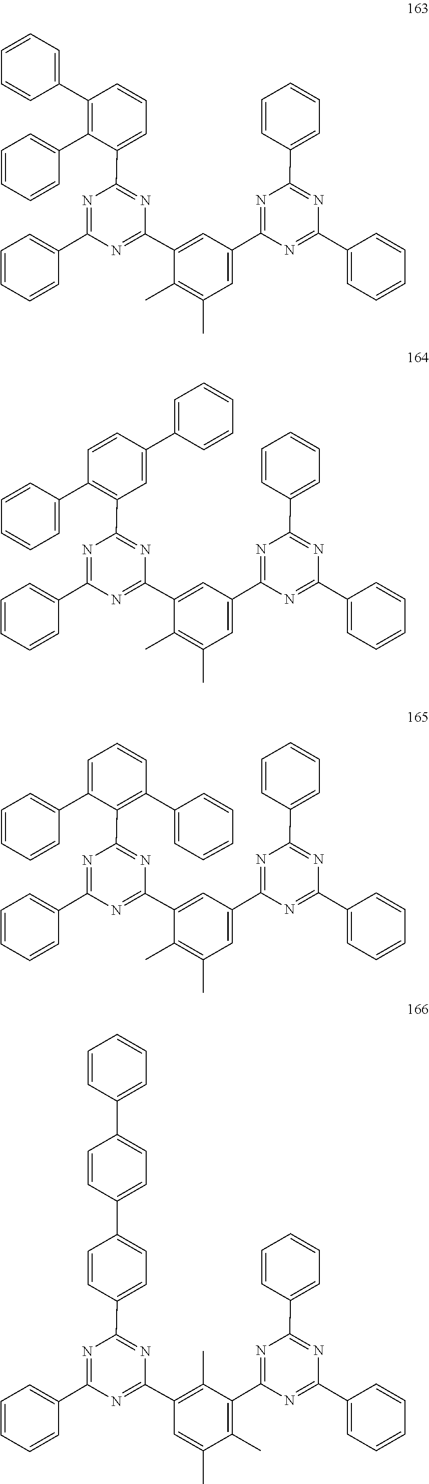 [SMILES: Cc1cc(-c2nc(-c3ccccc3)nc(-c3ccccc3)n2)cc(-c2nc(-c3ccccc3)nc(-c3cccc(-c4ccccc4)c3-c3ccccc3)n2)c1C ; Cc1cc(-c2nc(-c3ccccc3)nc(-c3ccccc3)n2)cc(-c2nc(-c3ccccc3)nc(-c3cc(-c4ccccc4)ccc3-c3ccccc3)n2)c1C ; Cc1cc(-c2nc(-c3ccccc3)nc(-c3ccccc3)n2)cc(-c2nc(-c3ccccc3)nc(-c3c(-c4ccccc4)cccc3-c3ccccc3)n2)c1C ; Cc1cc(-c2nc(-c3ccccc3)nc(-c3ccc(-c4ccc(-c5ccccc5)cc4)cc3)n2)c(C)c(-c2nc(-c3ccccc3)nc(-c3ccccc3)n2)c1C]

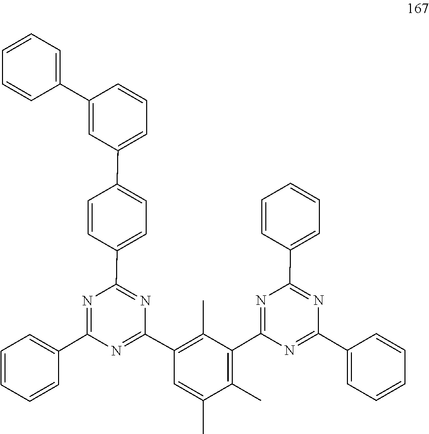 [SMILES: Cc1cc(-c2nc(-c3ccccc3)nc(-c3ccc(-c4cccc(-c5ccccc5)c4)cc3)n2)c(C)c(-c2nc(-c3ccccc3)nc(-c3ccccc3)n2)c1C]

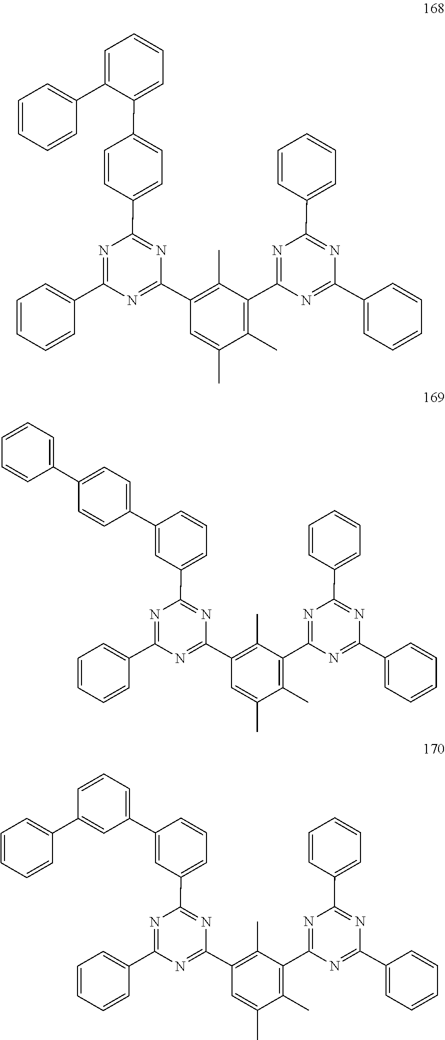 [SMILES: Cc1cc(-c2nc(-c3ccccc3)nc(-c3ccc(-c4ccccc4-c4ccccc4)cc3)n2)c(C)c(-c2nc(-c3ccccc3)nc(-c3ccccc3)n2)c1C ; Cc1cc(-c2nc(-c3ccccc3)nc(-c3cccc(-c4ccc(-c5ccccc5)cc4)c3)n2)c(C)c(-c2nc(-c3ccccc3)nc(-c3ccccc3)n2)c1C ; Cc1cc(-c2nc(-c3ccccc3)nc(-c3cccc(-c4cccc(-c5ccccc5)c4)c3)n2)c(C)c(-c2nc(-c3ccccc3)nc(-c3ccccc3)n2)c1C]

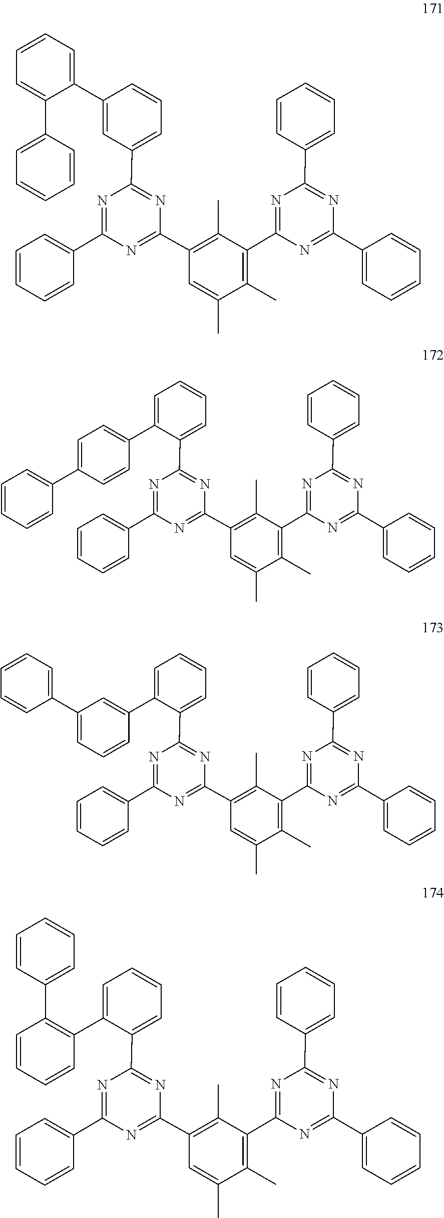 [SMILES: Cc1cc(-c2nc(-c3ccccc3)nc(-c3cccc(-c4ccccc4-c4ccccc4)c3)n2)c(C)c(-c2nc(-c3ccccc3)nc(-c3ccccc3)n2)c1C ; Cc1cc(-c2nc(-c3ccccc3)nc(-c3ccccc3-c3ccc(-c4ccccc4)cc3)n2)c(C)c(-c2nc(-c3ccccc3)nc(-c3ccccc3)n2)c1C ; Cc1cc(-c2nc(-c3ccccc3)nc(-c3ccccc3-c3cccc(-c4ccccc4)c3)n2)c(C)c(-c2nc(-c3ccccc3)nc(-c3ccccc3)n2)c1C ; Cc1cc(-c2nc(-c3ccccc3)nc(-c3ccccc3-c3ccccc3-c3ccccc3)n2)c(C)c(-c2nc(-c3ccccc3)nc(-c3ccccc3)n2)c1C]

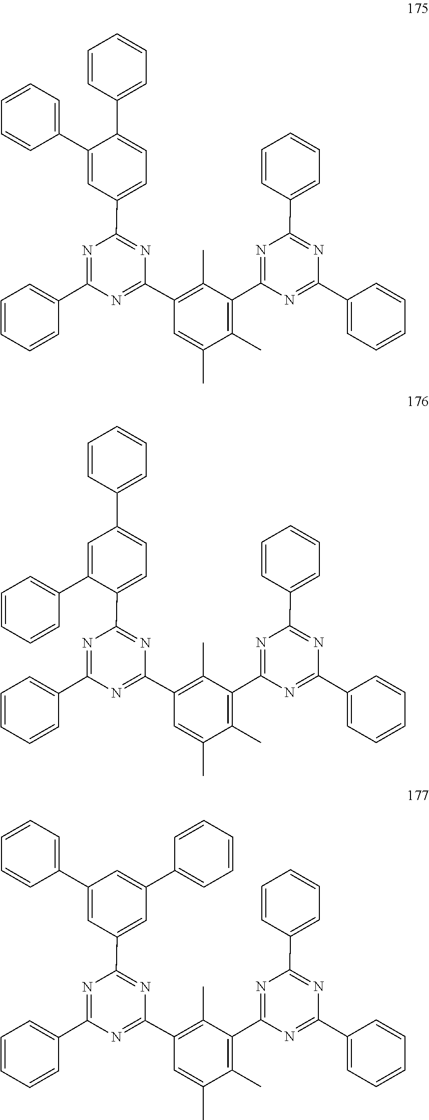

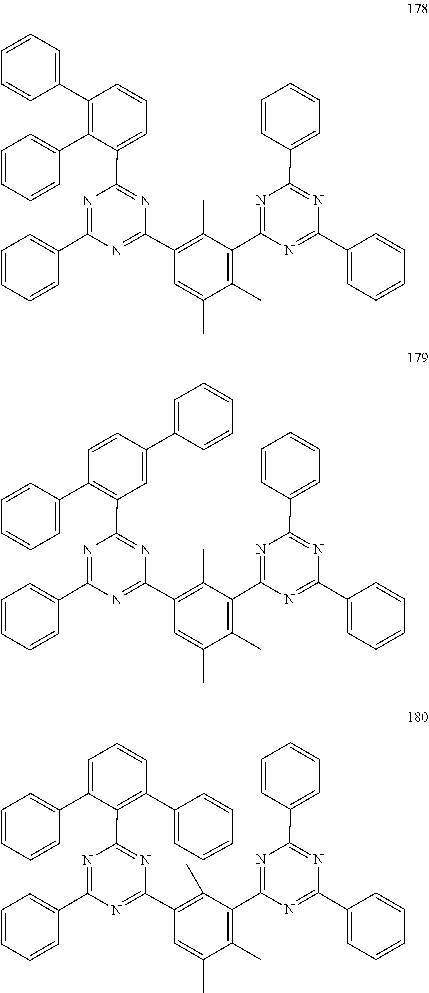 [SMILES: Cc1cc(-c2nc(-c3ccccc3)nc(-c3cccc(-c4ccccc4)c3-c3ccccc3)n2)c(C)c(-c2nc(-c3ccccc3)nc(-c3ccccc3)n2)c1C ; Cc1cc(-c2nc(-c3ccccc3)nc(-c3cc(-c4ccccc4)ccc3-c3ccccc3)n2)c(C)c(-c2nc(-c3ccccc3)nc(-c3ccccc3)n2)c1C ; Cc1cc(-c2nc(-c3ccccc3)nc(-c3c(-c4ccccc4)cccc3-c3ccccc3)n2)c(C)c(-c2nc(-c3ccccc3)nc(-c3ccccc3)n2)c1C]

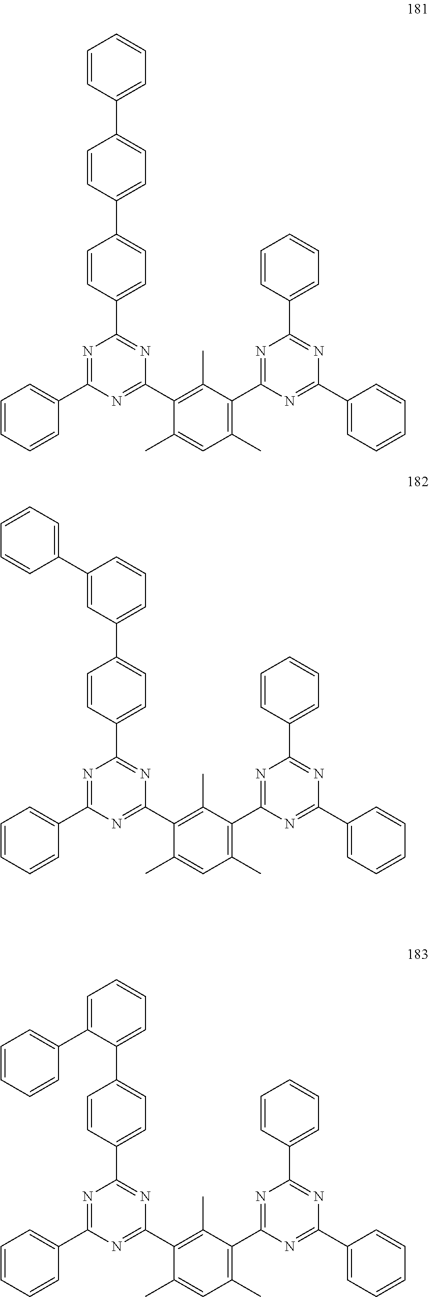 [SMILES: Cc1cc(C)c(-c2nc(-c3ccccc3)nc(-c3ccc(-c4ccc(-c5ccccc5)cc4)cc3)n2)c(C)c1-c1nc(-c2ccccc2)nc(-c2ccccc2)n1 ; Cc1cc(C)c(-c2nc(-c3ccccc3)nc(-c3ccc(-c4cccc(-c5ccccc5)c4)cc3)n2)c(C)c1-c1nc(-c2ccccc2)nc(-c2ccccc2)n1 ; Cc1cc(C)c(-c2nc(-c3ccccc3)nc(-c3ccc(-c4ccccc4-c4ccccc4)cc3)n2)c(C)c1-c1nc(-c2ccccc2)nc(-c2ccccc2)n1]

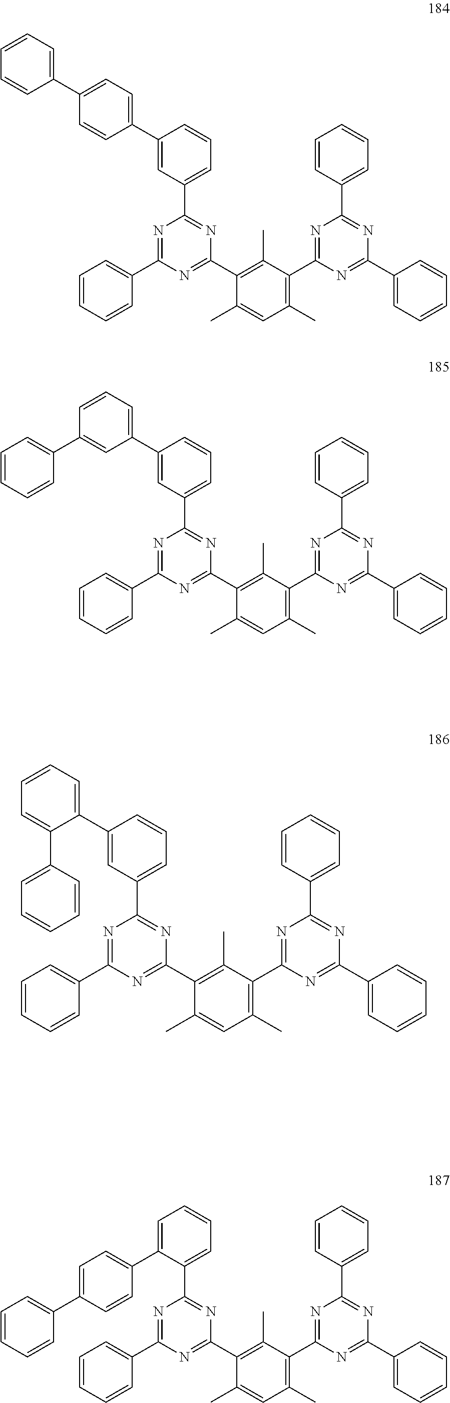 [SMILES: Cc1cc(C)c(-c2nc(-c3ccccc3)nc(-c3cccc(-c4ccc(-c5ccccc5)cc4)c3)n2)c(C)c1-c1nc(-c2ccccc2)nc(-c2ccccc2)n1 ; Cc1cc(C)c(-c2nc(-c3ccccc3)nc(-c3cccc(-c4cccc(-c5ccccc5)c4)c3)n2)c(C)c1-c1nc(-c2ccccc2)nc(-c2ccccc2)n1 ; Cc1cc(C)c(-c2nc(-c3ccccc3)nc(-c3cccc(-c4ccccc4-c4ccccc4)c3)n2)c(C)c1-c1nc(-c2ccccc2)nc(-c2ccccc2)n1 ; Cc1cc(C)c(-c2nc(-c3ccccc3)nc(-c3ccccc3-c3ccc(-c4ccccc4)cc3)n2)c(C)c1-c1nc(-c2ccccc2)nc(-c2ccccc2)n1]

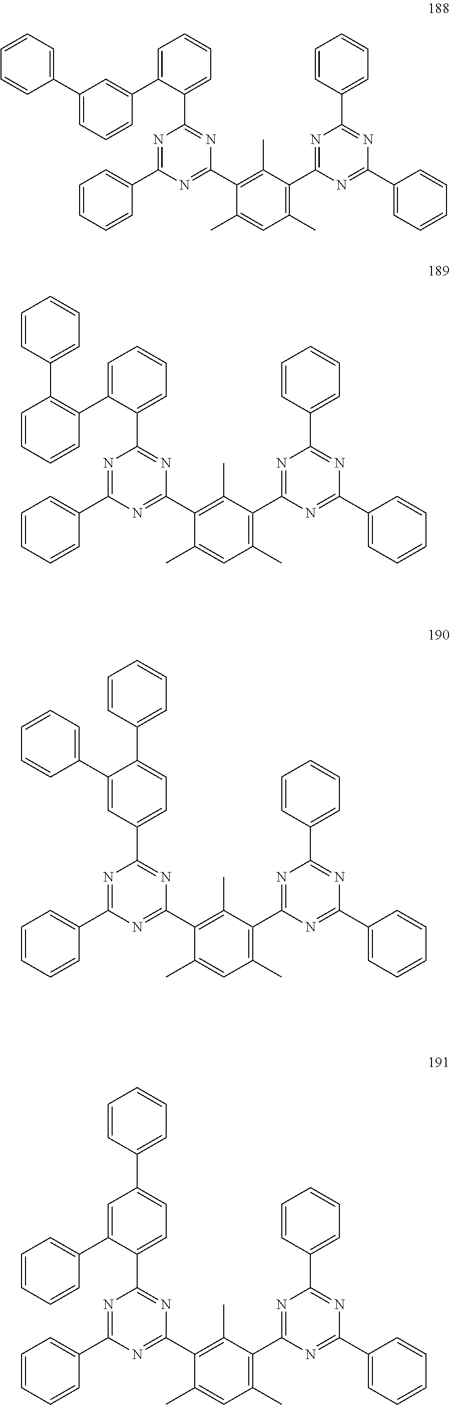 [SMILES: Cc1cc(C)c(-c2nc(-c3ccccc3)nc(-c3ccccc3-c3cccc(-c4ccccc4)c3)n2)c(C)c1-c1nc(-c2ccccc2)nc(-c2ccccc2)n1 ; Cc1cc(C)c(-c2nc(-c3ccccc3)nc(-c3ccccc3-c3ccccc3-c3ccccc3)n2)c(C)c1-c1nc(-c2ccccc2)nc(-c2ccccc2)n1 ; Cc1cc(C)c(-c2nc(-c3ccccc3)nc(-c3ccc(-c4ccccc4)c(-c4ccccc4)c3)n2)c(C)c1-c1nc(-c2ccccc2)nc(-c2ccccc2)n1 ; Cc1cc(C)c(-c2nc(-c3ccccc3)nc(-c3ccc(-c4ccccc4)cc3-c3ccccc3)n2)c(C)c1-c1nc(-c2ccccc2)nc(-c2ccccc2)n1]

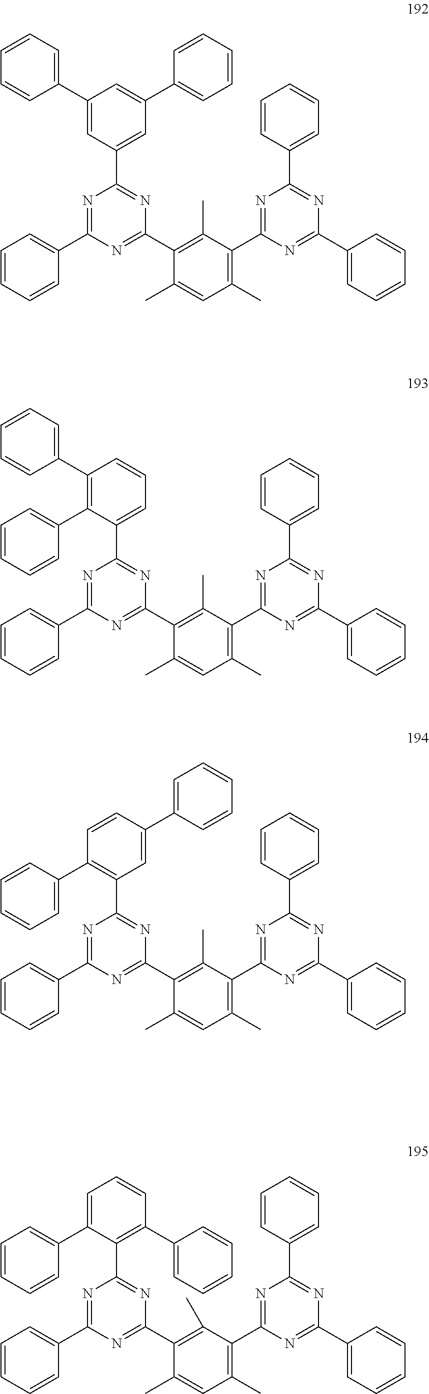 [SMILES: Cc1cc(C)c(-c2nc(-c3ccccc3)nc(-c3cc(-c4ccccc4)cc(-c4ccccc4)c3)n2)c(C)c1-c1nc(-c2ccccc2)nc(-c2ccccc2)n1 ; Cc1cc(C)c(-c2nc(-c3ccccc3)nc(-c3cccc(-c4ccccc4)c3-c3ccccc3)n2)c(C)c1-c1nc(-c2ccccc2)nc(-c2ccccc2)n1 ; Cc1cc(C)c(-c2nc(-c3ccccc3)nc(-c3cc(-c4ccccc4)ccc3-c3ccccc3)n2)c(C)c1-c1nc(-c2ccccc2)nc(-c2ccccc2)n1 ; Cc1cc(C)c(-c2nc(-c3ccccc3)nc(-c3c(-c4ccccc4)cccc3-c3ccccc3)n2)c(C)c1-c1nc(-c2ccccc2)nc(-c2ccccc2)n1]

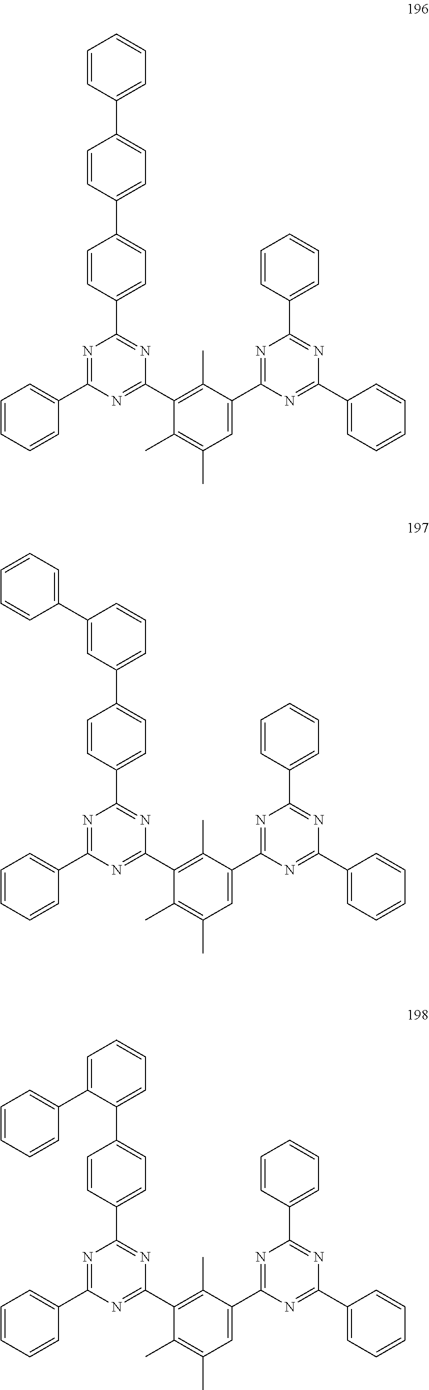 [SMILES: Cc1cc(-c2nc(-c3ccccc3)nc(-c3ccccc3)n2)c(C)c(-c2nc(-c3ccccc3)nc(-c3ccc(-c4ccc(-c5ccccc5)cc4)cc3)n2)c1C ; Cc1cc(-c2nc(-c3ccccc3)nc(-c3ccccc3)n2)c(C)c(-c2nc(-c3ccccc3)nc(-c3ccc(-c4cccc(-c5ccccc5)c4)cc3)n2)c1C ; Cc1cc(-c2nc(-c3ccccc3)nc(-c3ccccc3)n2)c(C)c(-c2nc(-c3ccccc3)nc(-c3ccc(-c4ccccc4-c4ccccc4)cc3)n2)c1C]

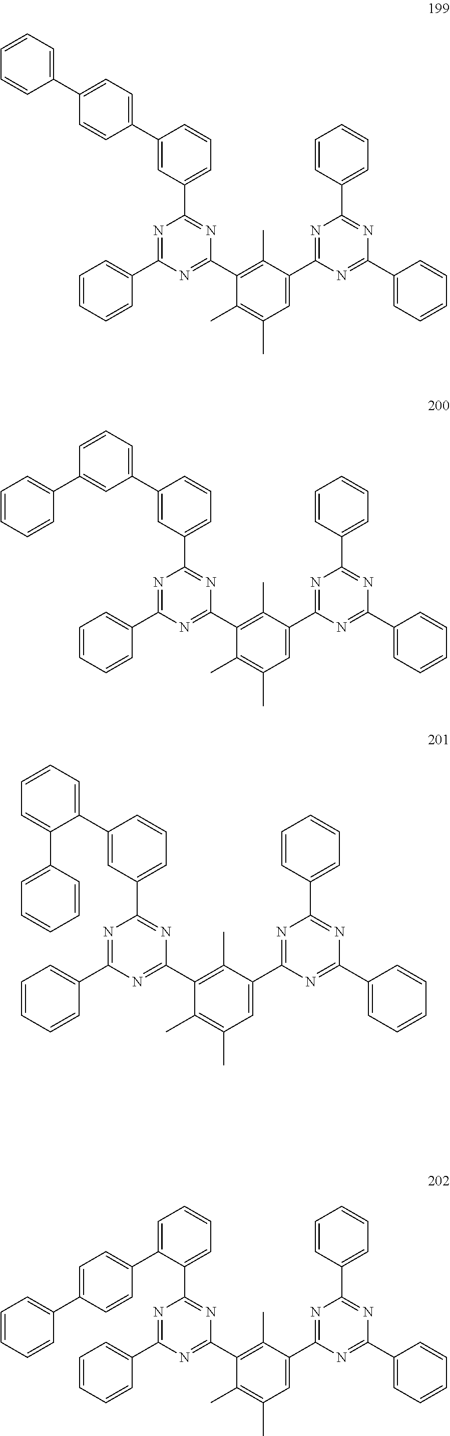 [SMILES: Cc1cc(-c2nc(-c3ccccc3)nc(-c3ccccc3)n2)c(C)c(-c2nc(-c3ccccc3)nc(-c3cccc(-c4ccc(-c5ccccc5)cc4)c3)n2)c1C ; Cc1cc(-c2nc(-c3ccccc3)nc(-c3ccccc3)n2)c(C)c(-c2nc(-c3ccccc3)nc(-c3cccc(-c4cccc(-c5ccccc5)c4)c3)n2)c1C ; Cc1cc(-c2nc(-c3ccccc3)nc(-c3ccccc3)n2)c(C)c(-c2nc(-c3ccccc3)nc(-c3cccc(-c4ccccc4-c4ccccc4)c3)n2)c1C ; Cc1cc(-c2nc(-c3ccccc3)nc(-c3ccccc3)n2)c(C)c(-c2nc(-c3ccccc3)nc(-c3ccccc3-c3ccc(-c4ccccc4)cc3)n2)c1C]

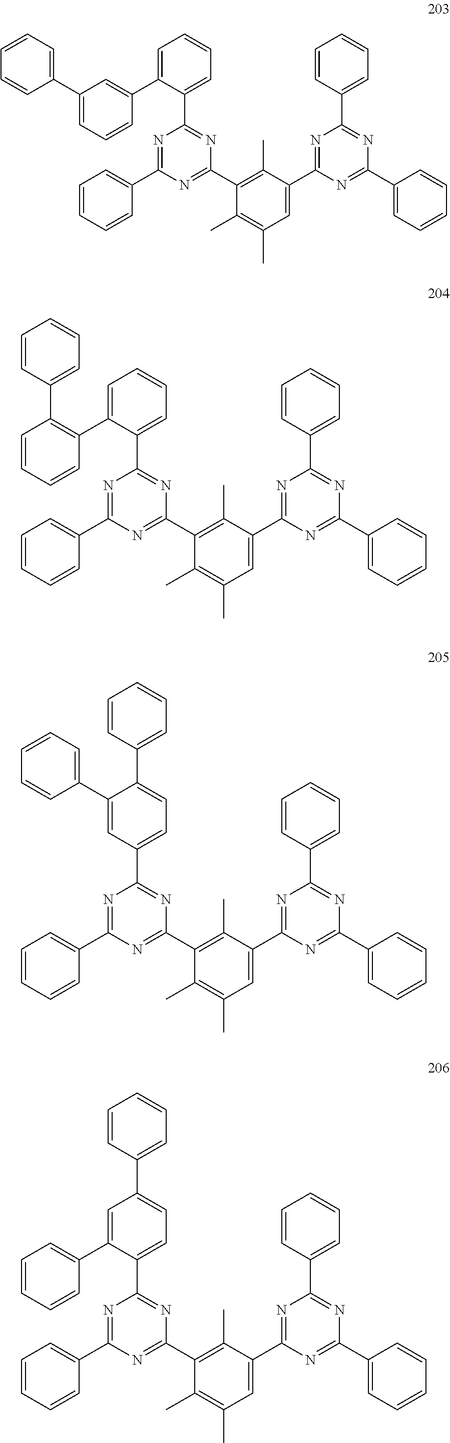 [SMILES: Cc1cc(-c2nc(-c3ccccc3)nc(-c3ccccc3)n2)c(C)c(-c2nc(-c3ccccc3)nc(-c3ccccc3-c3cccc(-c4ccccc4)c3)n2)c1C ; Cc1cc(-c2nc(-c3ccccc3)nc(-c3ccccc3)n2)c(C)c(-c2nc(-c3ccccc3)nc(-c3ccccc3-c3ccccc3-c3ccccc3)n2)c1C ; Cc1cc(-c2nc(-c3ccccc3)nc(-c3ccccc3)n2)c(C)c(-c2nc(-c3ccccc3)nc(-c3ccc(-c4ccccc4)c(-c4ccccc4)c3)n2)c1C ; Cc1cc(-c2nc(-c3ccccc3)nc(-c3ccccc3)n2)c(C)c(-c2nc(-c3ccccc3)nc(-c3ccc(-c4ccccc4)cc3-c3ccccc3)n2)c1C]

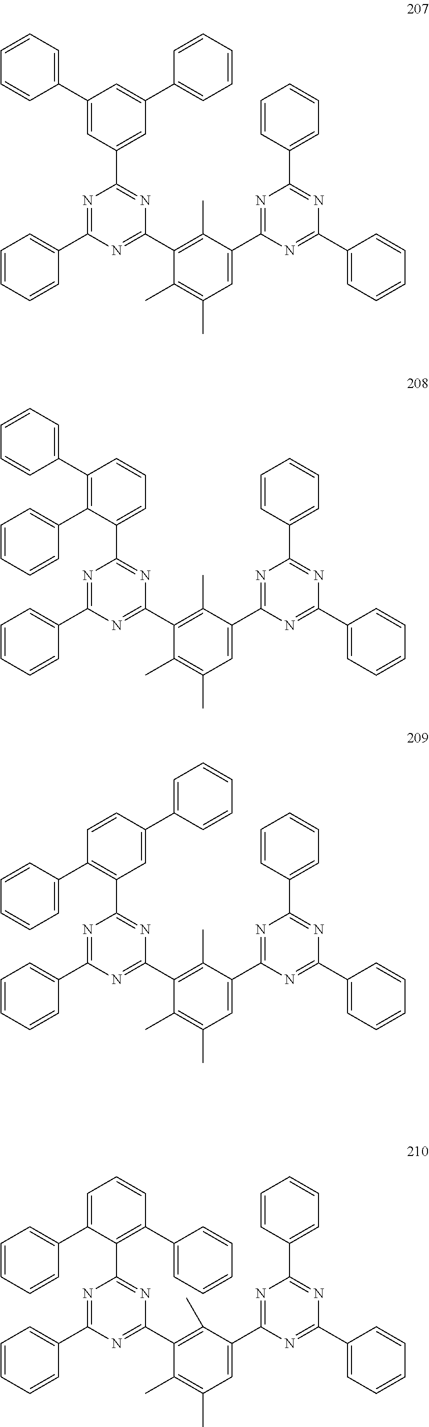 [SMILES: Cc1cc(-c2nc(-c3ccccc3)nc(-c3ccccc3)n2)c(C)c(-c2nc(-c3ccccc3)nc(-c3cc(-c4ccccc4)cc(-c4ccccc4)c3)n2)c1C ; Cc1cc(-c2nc(-c3ccccc3)nc(-c3ccccc3)n2)c(C)c(-c2nc(-c3ccccc3)nc(-c3cccc(-c4ccccc4)c3-c3ccccc3)n2)c1C ; Cc1cc(-c2nc(-c3ccccc3)nc(-c3ccccc3)n2)c(C)c(-c2nc(-c3ccccc3)nc(-c3cc(-c4ccccc4)ccc3-c3ccccc3)n2)c1C ; Cc1cc(-c2nc(-c3ccccc3)nc(-c3ccccc3)n2)c(C)c(-c2nc(-c3ccccc3)nc(-c3c(-c4ccccc4)cccc3-c3ccccc3)n2)c1C]

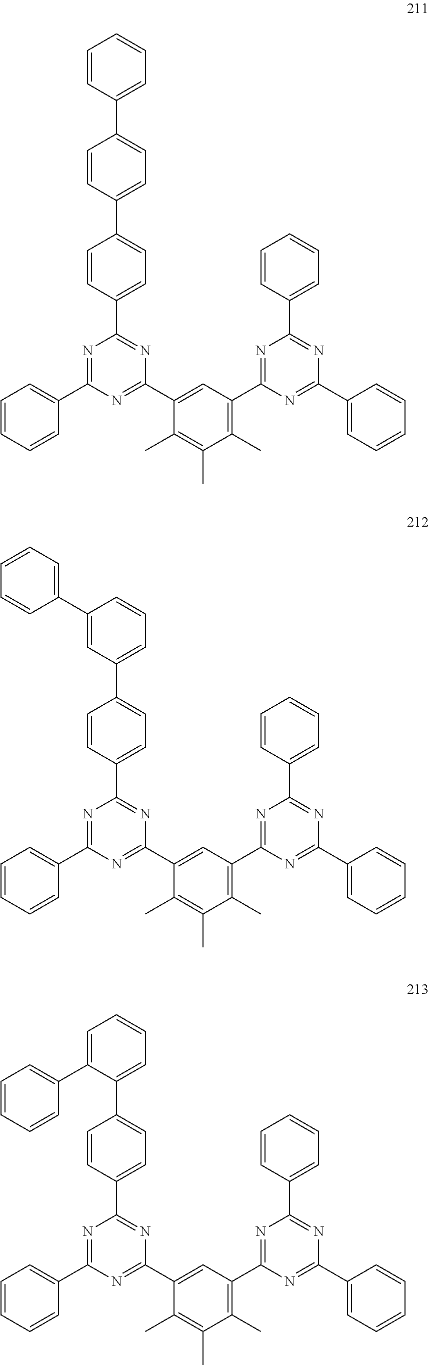 [SMILES: Cc1c(-c2nc(-c3ccccc3)nc(-c3ccccc3)n2)cc(-c2nc(-c3ccccc3)nc(-c3ccc(-c4ccc(-c5ccccc5)cc4)cc3)n2)c(C)c1C ; Cc1c(-c2nc(-c3ccccc3)nc(-c3ccccc3)n2)cc(-c2nc(-c3ccccc3)nc(-c3ccc(-c4cccc(-c5ccccc5)c4)cc3)n2)c(C)c1C ; Cc1c(-c2nc(-c3ccccc3)nc(-c3ccccc3)n2)cc(-c2nc(-c3ccccc3)nc(-c3ccc(-c4ccccc4-c4ccccc4)cc3)n2)c(C)c1C]

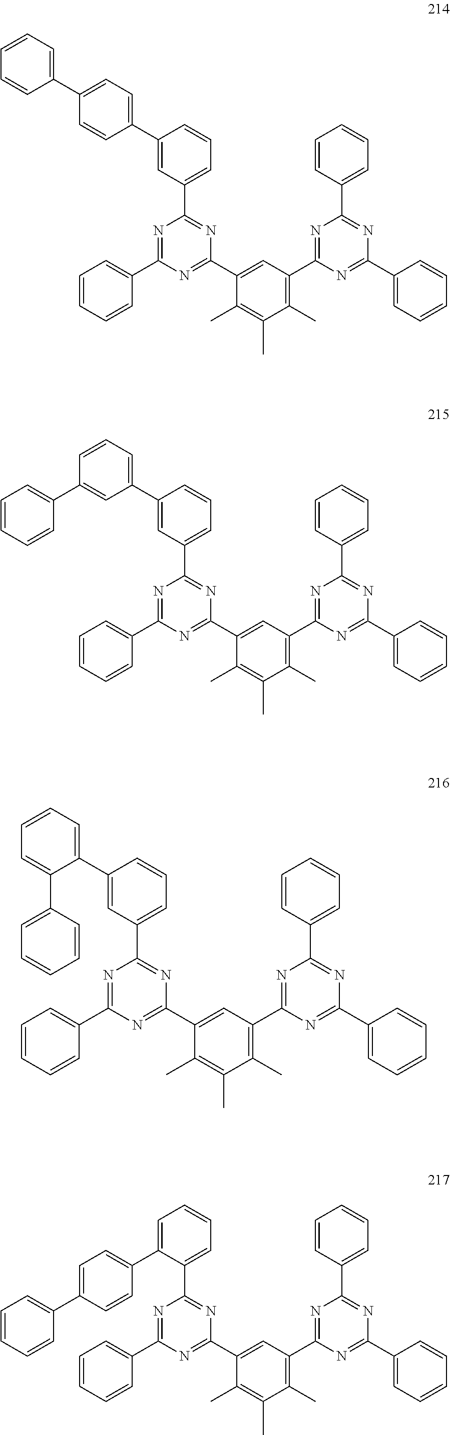 [SMILES: Cc1c(-c2nc(-c3ccccc3)nc(-c3ccccc3)n2)cc(-c2nc(-c3ccccc3)nc(-c3cccc(-c4ccc(-c5ccccc5)cc4)c3)n2)c(C)c1C ; Cc1c(-c2nc(-c3ccccc3)nc(-c3ccccc3)n2)cc(-c2nc(-c3ccccc3)nc(-c3cccc(-c4cccc(-c5ccccc5)c4)c3)n2)c(C)c1C ; Cc1c(-c2nc(-c3ccccc3)nc(-c3ccccc3)n2)cc(-c2nc(-c3ccccc3)nc(-c3cccc(-c4ccccc4-c4ccccc4)c3)n2)c(C)c1C ; Cc1c(-c2nc(-c3ccccc3)nc(-c3ccccc3)n2)cc(-c2nc(-c3ccccc3)nc(-c3ccccc3-c3ccc(-c4ccccc4)cc3)n2)c(C)c1C]

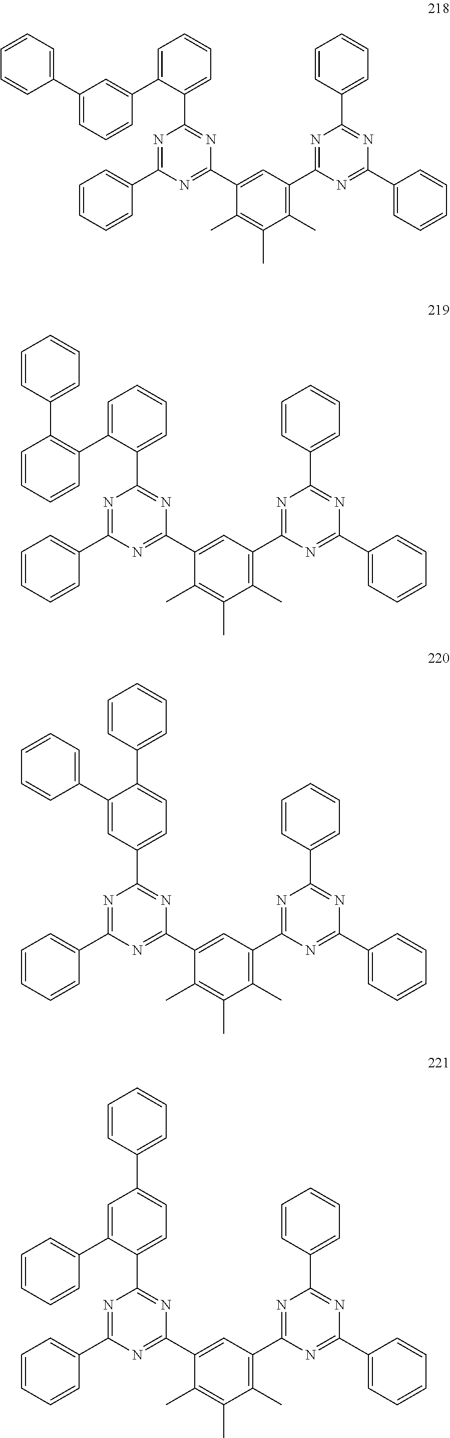 [SMILES: Cc1c(-c2nc(-c3ccccc3)nc(-c3ccccc3)n2)cc(-c2nc(-c3ccccc3)nc(-c3ccccc3-c3cccc(-c4ccccc4)c3)n2)c(C)c1C ; Cc1c(-c2nc(-c3ccccc3)nc(-c3ccccc3)n2)cc(-c2nc(-c3ccccc3)nc(-c3ccccc3-c3ccccc3-c3ccccc3)n2)c(C)c1C ; Cc1c(-c2nc(-c3ccccc3)nc(-c3ccccc3)n2)cc(-c2nc(-c3ccccc3)nc(-c3ccc(-c4ccccc4)c(-c4ccccc4)c3)n2)c(C)c1C ; Cc1c(-c2nc(-c3ccccc3)nc(-c3ccccc3)n2)cc(-c2nc(-c3ccccc3)nc(-c3ccc(-c4ccccc4)cc3-c3ccccc3)n2)c(C)c1C]

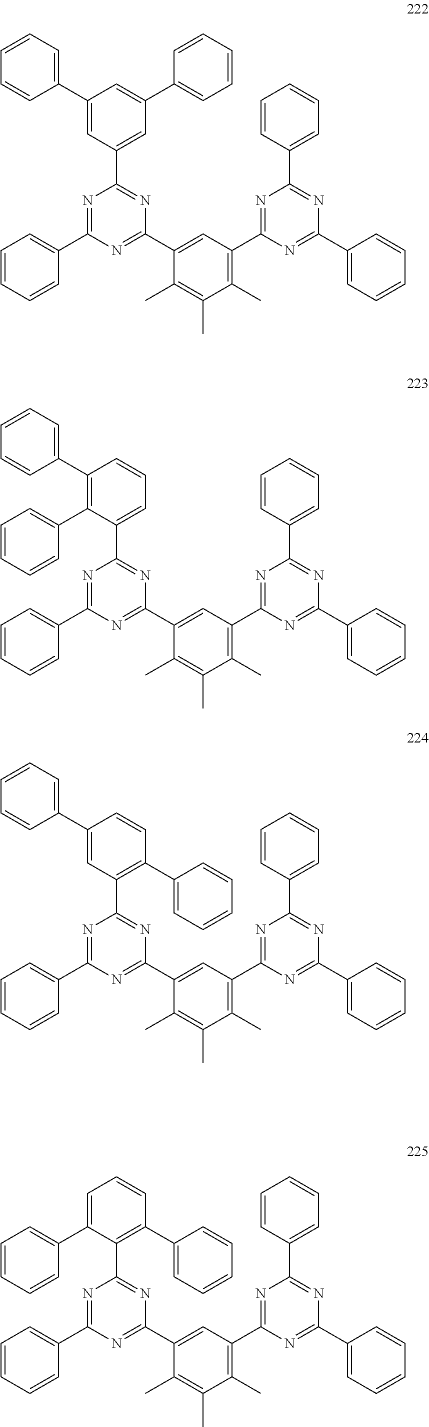 [SMILES: Cc1c(-c2nc(-c3ccccc3)nc(-c3ccccc3)n2)cc(-c2nc(-c3ccccc3)nc(-c3cc(-c4ccccc4)cc(-c4ccccc4)c3)n2)c(C)c1C ; Cc1c(-c2nc(-c3ccccc3)nc(-c3ccccc3)n2)cc(-c2nc(-c3ccccc3)nc(-c3cccc(-c4ccccc4)c3-c3ccccc3)n2)c(C)c1C ; Cc1c(-c2nc(-c3ccccc3)nc(-c3ccccc3)n2)cc(-c2nc(-c3ccccc3)nc(-c3cc(-c4ccccc4)ccc3-c3ccccc3)n2)c(C)c1C ; Cc1c(-c2nc(-c3ccccc3)nc(-c3ccccc3)n2)cc(-c2nc(-c3ccccc3)nc(-c3c(-c4ccccc4)cccc3-c3ccccc3)n2)c(C)c1C]

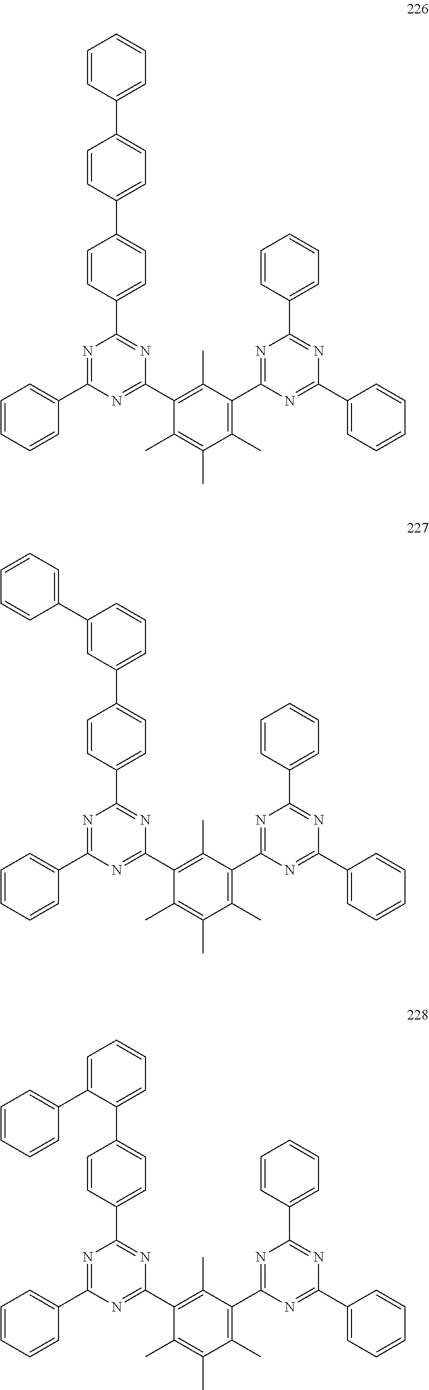 [SMILES: Cc1c(C)c(-c2nc(-c3ccccc3)nc(-c3ccccc3)n2)c(C)c(-c2nc(-c3ccccc3)nc(-c3ccc(-c4ccc(-c5ccccc5)cc4)cc3)n2)c1C ; Cc1c(C)c(-c2nc(-c3ccccc3)nc(-c3ccccc3)n2)c(C)c(-c2nc(-c3ccccc3)nc(-c3ccc(-c4cccc(-c5ccccc5)c4)cc3)n2)c1C ; Cc1c(C)c(-c2nc(-c3ccccc3)nc(-c3ccccc3)n2)c(C)c(-c2nc(-c3ccccc3)nc(-c3ccc(-c4ccccc4-c4ccccc4)cc3)n2)c1C]

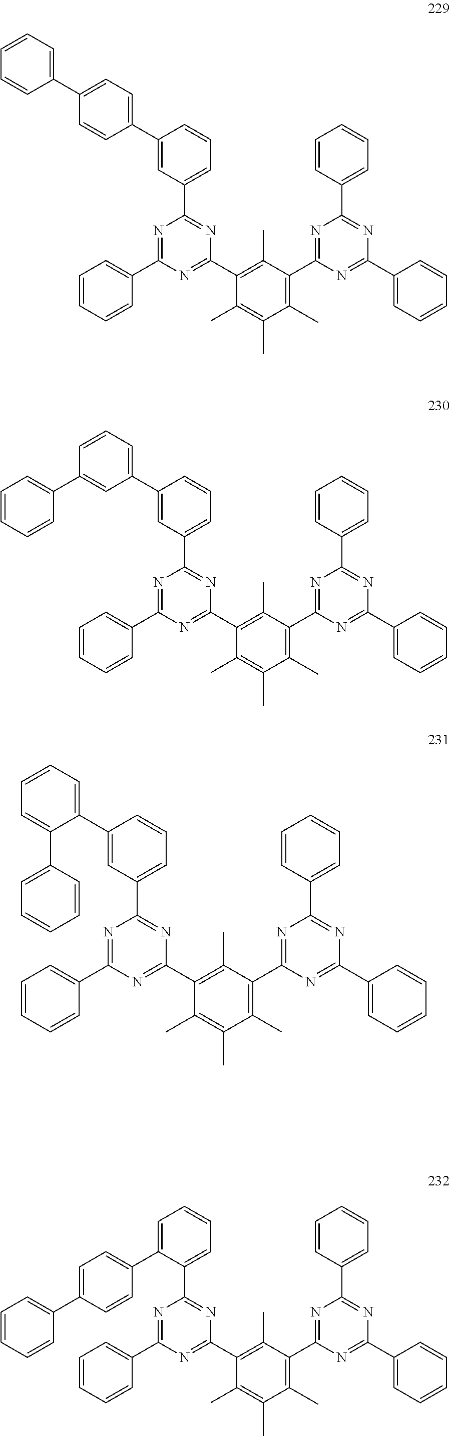 [SMILES: Cc1c(C)c(-c2nc(-c3ccccc3)nc(-c3ccccc3)n2)c(C)c(-c2nc(-c3ccccc3)nc(-c3cccc(-c4ccc(-c5ccccc5)cc4)c3)n2)c1C ; Cc1c(C)c(-c2nc(-c3ccccc3)nc(-c3ccccc3)n2)c(C)c(-c2nc(-c3ccccc3)nc(-c3cccc(-c4cccc(-c5ccccc5)c4)c3)n2)c1C ; Cc1c(C)c(-c2nc(-c3ccccc3)nc(-c3ccccc3)n2)c(C)c(-c2nc(-c3ccccc3)nc(-c3cccc(-c4ccccc4-c4ccccc4)c3)n2)c1C ; Cc1c(C)c(-c2nc(-c3ccccc3)nc(-c3ccccc3)n2)c(C)c(-c2nc(-c3ccccc3)nc(-c3ccccc3-c3ccc(-c4ccccc4)cc3)n2)c1C]

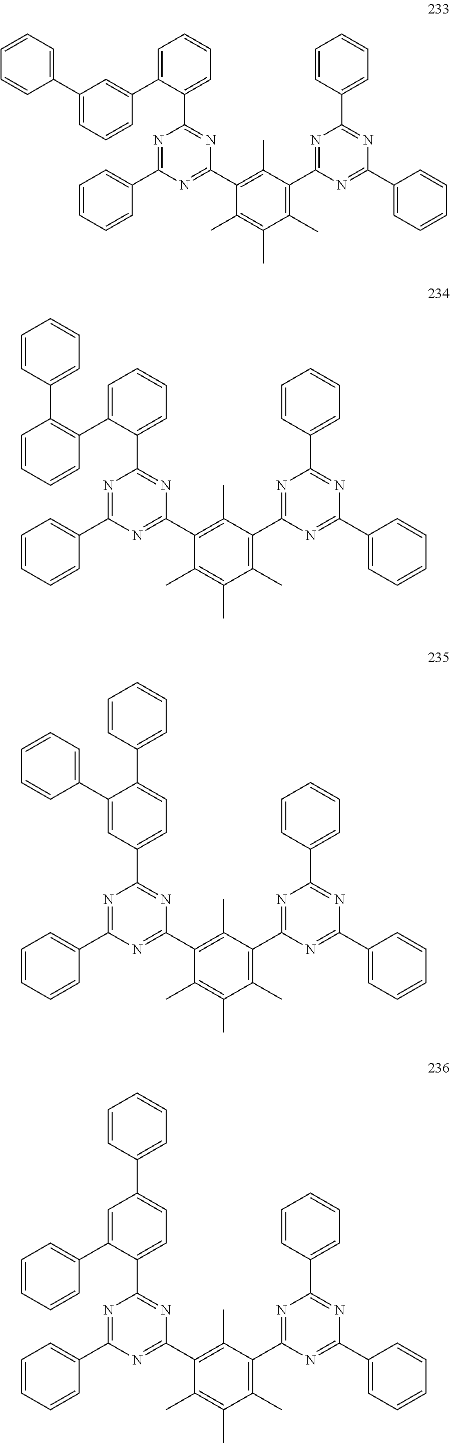 [SMILES: Cc1c(C)c(-c2nc(-c3ccccc3)nc(-c3ccccc3)n2)c(C)c(-c2nc(-c3ccccc3)nc(-c3ccccc3-c3cccc(-c4ccccc4)c3)n2)c1C ; Cc1c(C)c(-c2nc(-c3ccccc3)nc(-c3ccccc3)n2)c(C)c(-c2nc(-c3ccccc3)nc(-c3ccccc3-c3ccccc3-c3ccccc3)n2)c1C ; Cc1c(C)c(-c2nc(-c3ccccc3)nc(-c3ccccc3)n2)c(C)c(-c2nc(-c3ccccc3)nc(-c3ccc(-c4ccccc4)c(-c4ccccc4)c3)n2)c1C ; Cc1c(C)c(-c2nc(-c3ccccc3)nc(-c3ccccc3)n2)c(C)c(-c2nc(-c3ccccc3)nc(-c3ccc(-c4ccccc4)cc3-c3ccccc3)n2)c1C]

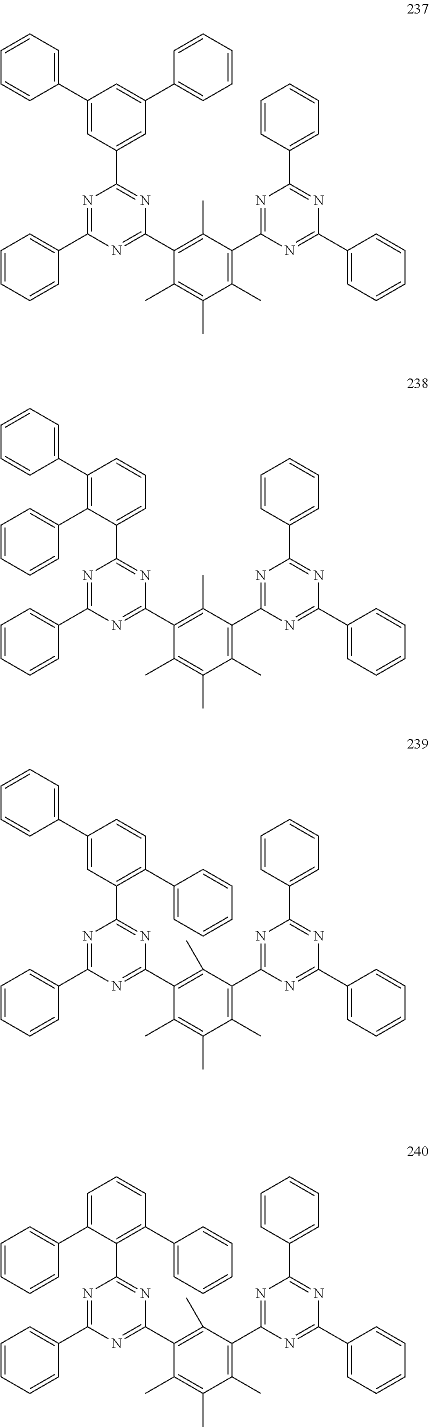 [SMILES: Cc1c(C)c(-c2nc(-c3ccccc3)nc(-c3ccccc3)n2)c(C)c(-c2nc(-c3ccccc3)nc(-c3cc(-c4ccccc4)cc(-c4ccccc4)c3)n2)c1C ; Cc1c(C)c(-c2nc(-c3ccccc3)nc(-c3ccccc3)n2)c(C)c(-c2nc(-c3ccccc3)nc(-c3cccc(-c4ccccc4)c3-c3ccccc3)n2)c1C ; Cc1c(C)c(-c2nc(-c3ccccc3)nc(-c3ccccc3)n2)c(C)c(-c2nc(-c3ccccc3)nc(-c3cc(-c4ccccc4)ccc3-c3ccccc3)n2)c1C ; Cc1c(C)c(-c2nc(-c3ccccc3)nc(-c3ccccc3)n2)c(C)c(-c2nc(-c3ccccc3)nc(-c3c(-c4ccccc4)cccc3-c3ccccc3)n2)c1C]

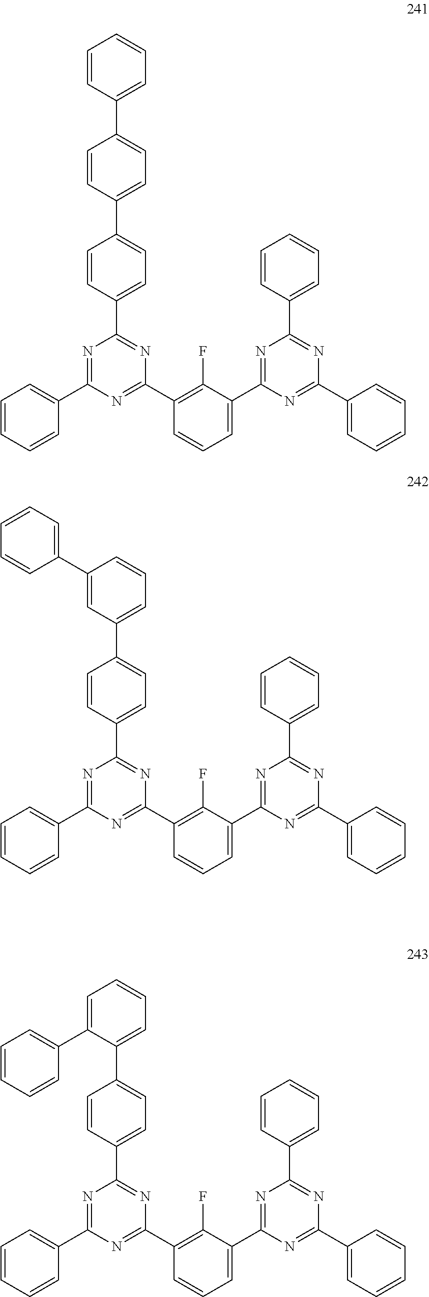 [SMILES: Fc1c(-c2nc(-c3ccccc3)nc(-c3ccccc3)n2)cccc1-c1nc(-c2ccccc2)nc(-c2ccc(-c3ccc(-c4ccccc4)cc3)cc2)n1 ; Fc1c(-c2nc(-c3ccccc3)nc(-c3ccccc3)n2)cccc1-c1nc(-c2ccccc2)nc(-c2ccc(-c3cccc(-c4ccccc4)c3)cc2)n1 ; Fc1c(-c2nc(-c3ccccc3)nc(-c3ccccc3)n2)cccc1-c1nc(-c2ccccc2)nc(-c2ccc(-c3ccccc3-c3ccccc3)cc2)n1]

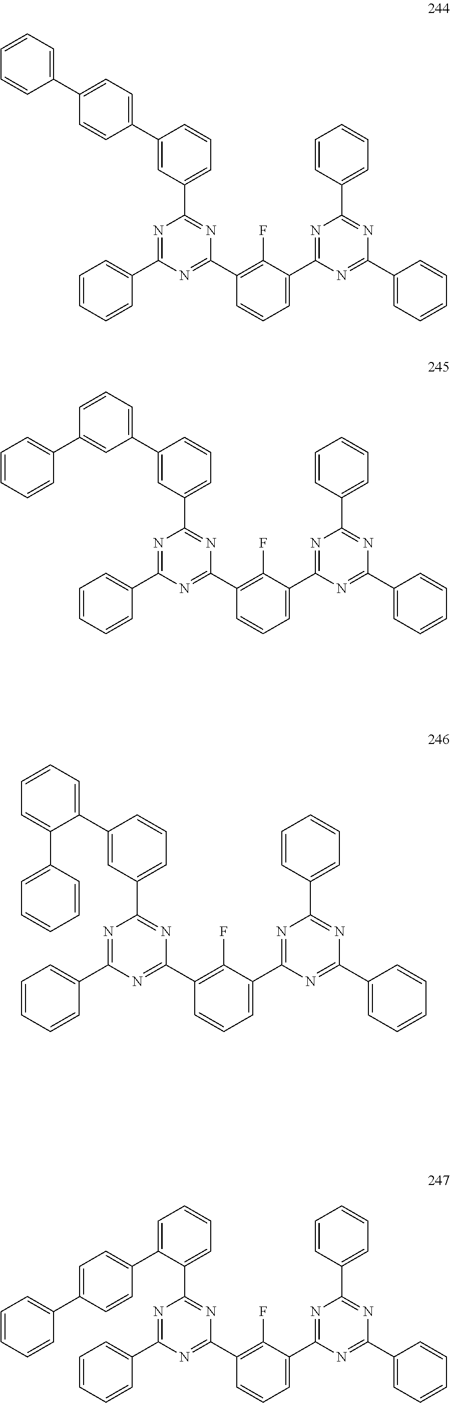 [SMILES: Fc1c(-c2nc(-c3ccccc3)nc(-c3ccccc3)n2)cccc1-c1nc(-c2ccccc2)nc(-c2cccc(-c3ccc(-c4ccccc4)cc3)c2)n1 ; Fc1c(-c2nc(-c3ccccc3)nc(-c3ccccc3)n2)cccc1-c1nc(-c2ccccc2)nc(-c2cccc(-c3cccc(-c4ccccc4)c3)c2)n1 ; Fc1c(-c2nc(-c3ccccc3)nc(-c3ccccc3)n2)cccc1-c1nc(-c2ccccc2)nc(-c2cccc(-c3ccccc3-c3ccccc3)c2)n1 ; Fc1c(-c2nc(-c3ccccc3)nc(-c3ccccc3)n2)cccc1-c1nc(-c2ccccc2)nc(-c2ccccc2-c2ccc(-c3ccccc3)cc2)n1]

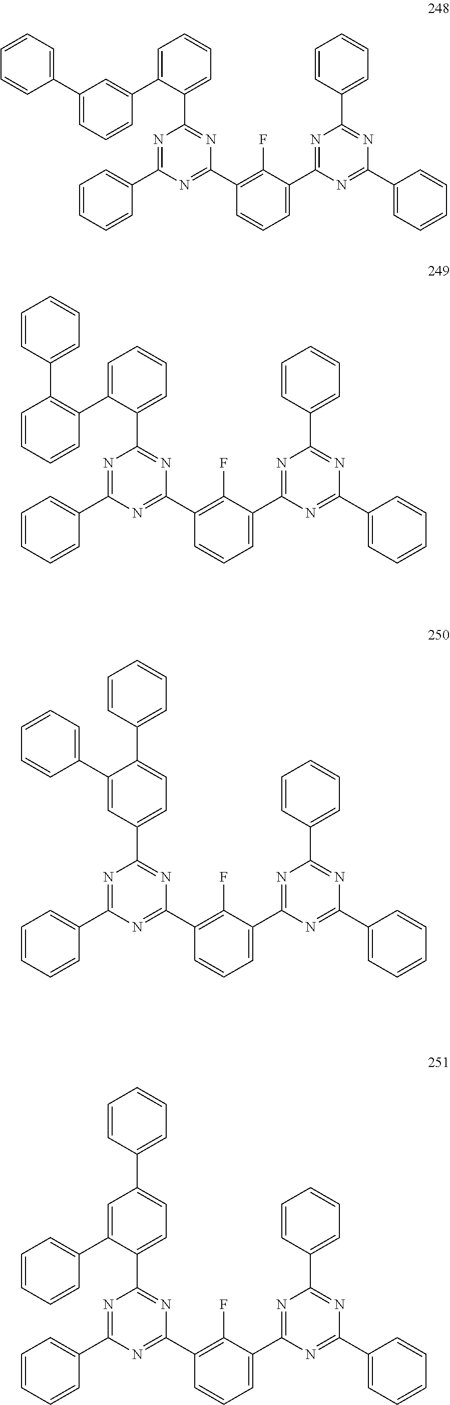 [SMILES: Fc1c(-c2nc(-c3ccccc3)nc(-c3ccccc3)n2)cccc1-c1nc(-c2ccccc2)nc(-c2ccccc2-c2cccc(-c3ccccc3)c2)n1 ; Fc1c(-c2nc(-c3ccccc3)nc(-c3ccccc3)n2)cccc1-c1nc(-c2ccccc2)nc(-c2ccccc2-c2ccccc2-c2ccccc2)n1 ; Fc1c(-c2nc(-c3ccccc3)nc(-c3ccccc3)n2)cccc1-c1nc(-c2ccccc2)nc(-c2ccc(-c3ccccc3)c(-c3ccccc3)c2)n1 ; Fc1c(-c2nc(-c3ccccc3)nc(-c3ccccc3)n2)cccc1-c1nc(-c2ccccc2)nc(-c2ccc(-c3ccccc3)cc2-c2ccccc2)n1]

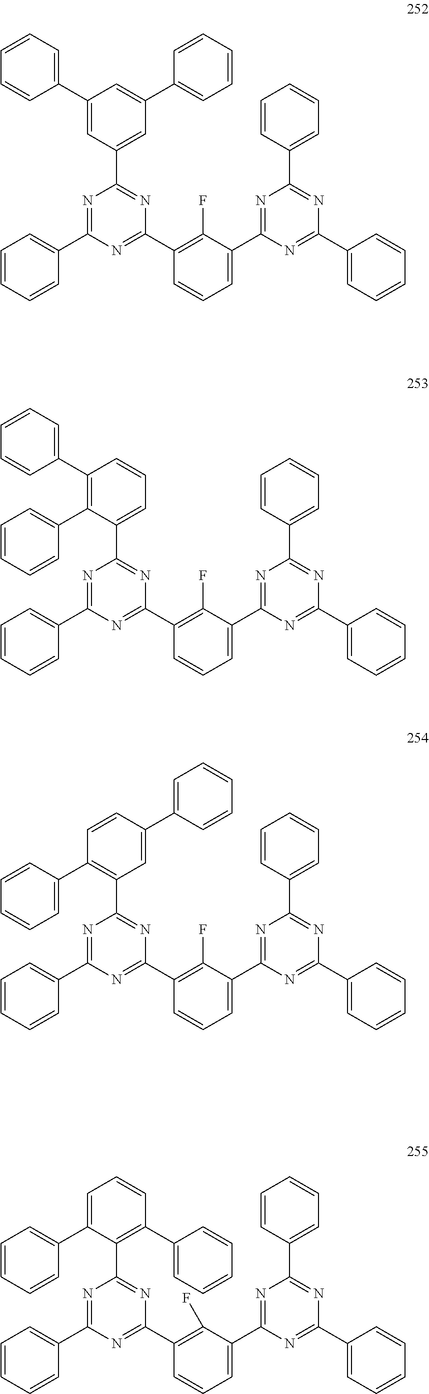 [SMILES: Fc1c(-c2nc(-c3ccccc3)nc(-c3ccccc3)n2)cccc1-c1nc(-c2ccccc2)nc(-c2cc(-c3ccccc3)cc(-c3ccccc3)c2)n1 ; Fc1c(-c2nc(-c3ccccc3)nc(-c3ccccc3)n2)cccc1-c1nc(-c2ccccc2)nc(-c2cccc(-c3ccccc3)c2-c2ccccc2)n1 ; Fc1c(-c2nc(-c3ccccc3)nc(-c3ccccc3)n2)cccc1-c1nc(-c2ccccc2)nc(-c2cc(-c3ccccc3)ccc2-c2ccccc2)n1 ; Fc1c(-c2nc(-c3ccccc3)nc(-c3ccccc3)n2)cccc1-c1nc(-c2ccccc2)nc(-c2c(-c3ccccc3)cccc2-c2ccccc2)n1]

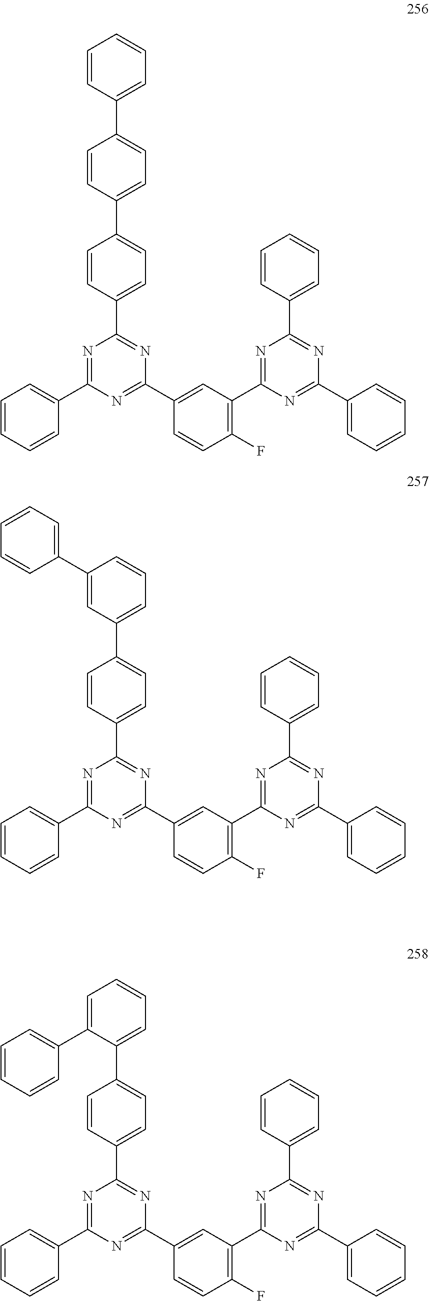 [SMILES: Fc1ccc(-c2nc(-c3ccccc3)nc(-c3ccc(-c4ccc(-c5ccccc5)cc4)cc3)n2)cc1-c1nc(-c2ccccc2)nc(-c2ccccc2)n1 ; Fc1ccc(-c2nc(-c3ccccc3)nc(-c3ccc(-c4cccc(-c5ccccc5)c4)cc3)n2)cc1-c1nc(-c2ccccc2)nc(-c2ccccc2)n1 ; Fc1ccc(-c2nc(-c3ccccc3)nc(-c3ccc(-c4ccccc4-c4ccccc4)cc3)n2)cc1-c1nc(-c2ccccc2)nc(-c2ccccc2)n1]

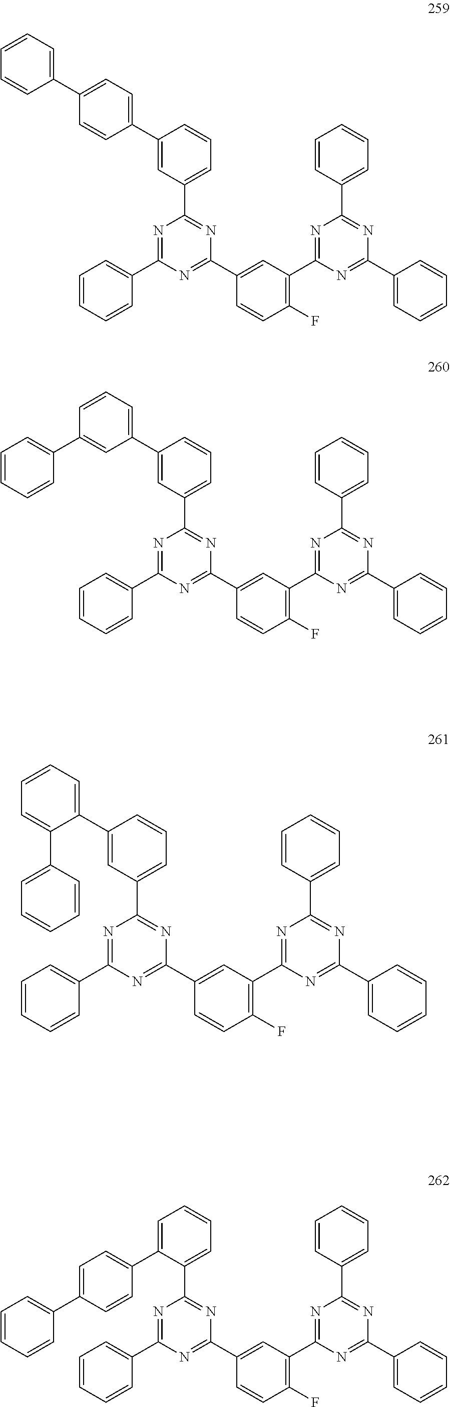 [SMILES: Fc1ccc(-c2nc(-c3ccccc3)nc(-c3cccc(-c4ccc(-c5ccccc5)cc4)c3)n2)cc1-c1nc(-c2ccccc2)nc(-c2ccccc2)n1 ; Fc1ccc(-c2nc(-c3ccccc3)nc(-c3cccc(-c4cccc(-c5ccccc5)c4)c3)n2)cc1-c1nc(-c2ccccc2)nc(-c2ccccc2)n1 ; Fc1ccc(-c2nc(-c3ccccc3)nc(-c3cccc(-c4ccccc4-c4ccccc4)c3)n2)cc1-c1nc(-c2ccccc2)nc(-c2ccccc2)n1 ; Fc1ccc(-c2nc(-c3ccccc3)nc(-c3ccccc3-c3ccc(-c4ccccc4)cc3)n2)cc1-c1nc(-c2ccccc2)nc(-c2ccccc2)n1]

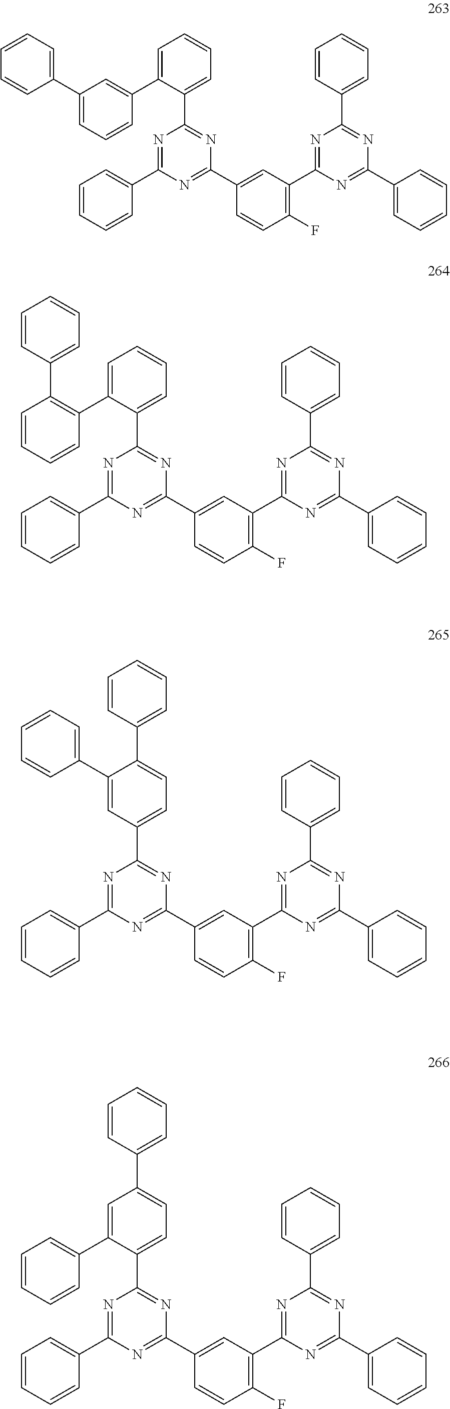 [SMILES: Fc1ccc(-c2nc(-c3ccccc3)nc(-c3ccccc3-c3cccc(-c4ccccc4)c3)n2)cc1-c1nc(-c2ccccc2)nc(-c2ccccc2)n1 ; Fc1ccc(-c2nc(-c3ccccc3)nc(-c3ccccc3-c3ccccc3-c3ccccc3)n2)cc1-c1nc(-c2ccccc2)nc(-c2ccccc2)n1 ; Fc1ccc(-c2nc(-c3ccccc3)nc(-c3ccc(-c4ccccc4)c(-c4ccccc4)c3)n2)cc1-c1nc(-c2ccccc2)nc(-c2ccccc2)n1 ; Fc1ccc(-c2nc(-c3ccccc3)nc(-c3ccc(-c4ccccc4)cc3-c3ccccc3)n2)cc1-c1nc(-c2ccccc2)nc(-c2ccccc2)n1]

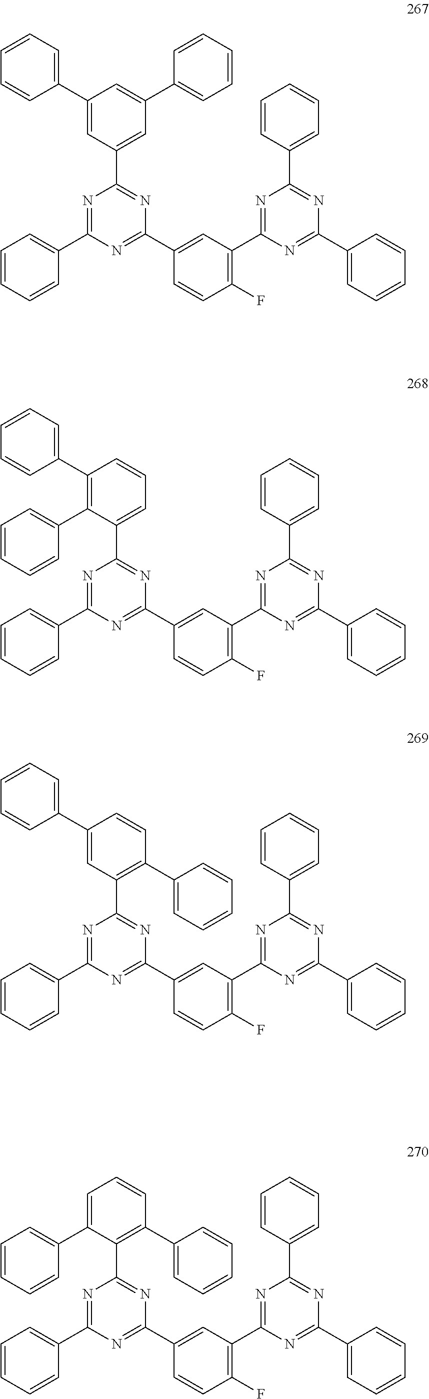 [SMILES: Fc1ccc(-c2nc(-c3ccccc3)nc(-c3cc(-c4ccccc4)cc(-c4ccccc4)c3)n2)cc1-c1nc(-c2ccccc2)nc(-c2ccccc2)n1 ; Fc1ccc(-c2nc(-c3ccccc3)nc(-c3cccc(-c4ccccc4)c3-c3ccccc3)n2)cc1-c1nc(-c2ccccc2)nc(-c2ccccc2)n1 ; Fc1ccc(-c2nc(-c3ccccc3)nc(-c3cc(-c4ccccc4)ccc3-c3ccccc3)n2)cc1-c1nc(-c2ccccc2)nc(-c2ccccc2)n1 ; Fc1ccc(-c2nc(-c3ccccc3)nc(-c3c(-c4ccccc4)cccc3-c3ccccc3)n2)cc1-c1nc(-c2ccccc2)nc(-c2ccccc2)n1]

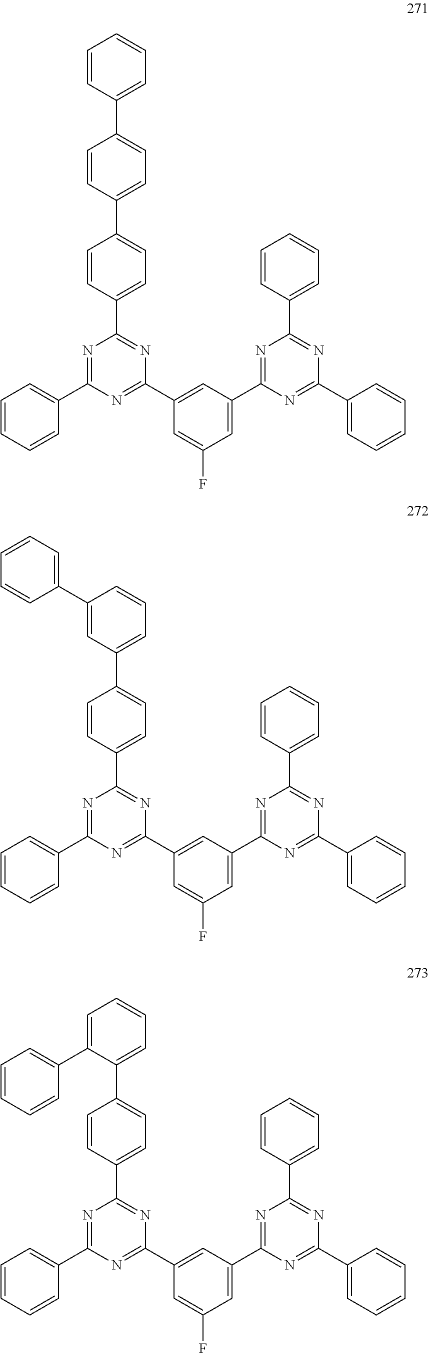 [SMILES: Fc1cc(-c2nc(-c3ccccc3)nc(-c3ccccc3)n2)cc(-c2nc(-c3ccccc3)nc(-c3ccc(-c4ccc(-c5ccccc5)cc4)cc3)n2)c1 ; Fc1cc(-c2nc(-c3ccccc3)nc(-c3ccccc3)n2)cc(-c2nc(-c3ccccc3)nc(-c3ccc(-c4cccc(-c5ccccc5)c4)cc3)n2)c1 ; Fc1cc(-c2nc(-c3ccccc3)nc(-c3ccccc3)n2)cc(-c2nc(-c3ccccc3)nc(-c3ccc(-c4ccccc4-c4ccccc4)cc3)n2)c1]

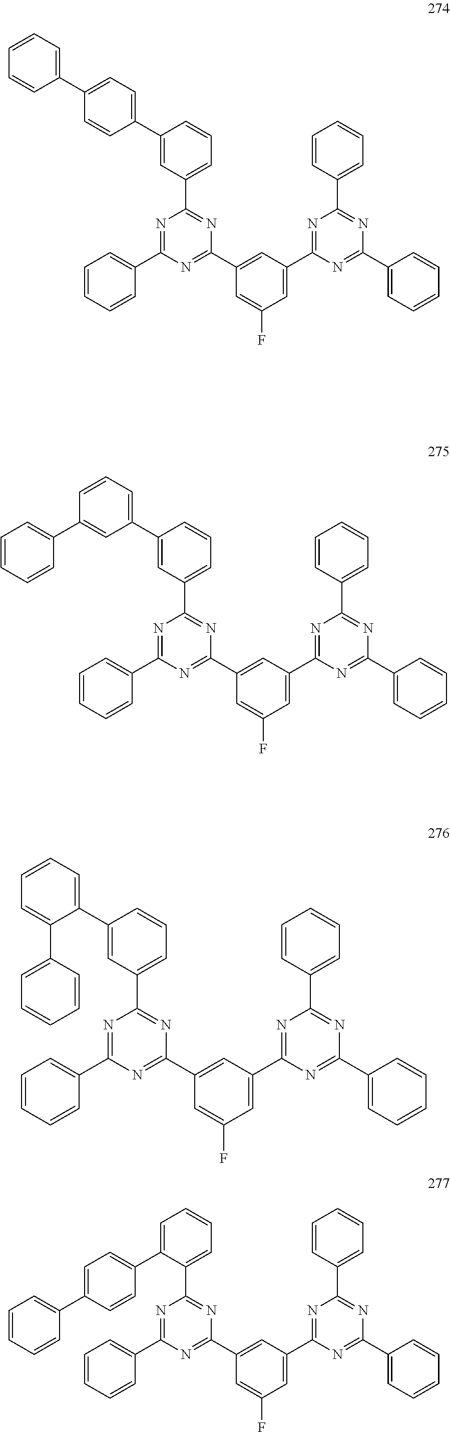 [SMILES: Fc1cc(-c2nc(-c3ccccc3)nc(-c3ccccc3)n2)cc(-c2nc(-c3ccccc3)nc(-c3cccc(-c4ccc(-c5ccccc5)cc4)c3)n2)c1 ; Fc1cc(-c2nc(-c3ccccc3)nc(-c3ccccc3)n2)cc(-c2nc(-c3ccccc3)nc(-c3cccc(-c4cccc(-c5ccccc5)c4)c3)n2)c1 ; Fc1cc(-c2nc(-c3ccccc3)nc(-c3ccccc3)n2)cc(-c2nc(-c3ccccc3)nc(-c3cccc(-c4ccccc4-c4ccccc4)c3)n2)c1 ; Fc1cc(-c2nc(-c3ccccc3)nc(-c3ccccc3)n2)cc(-c2nc(-c3ccccc3)nc(-c3ccccc3-c3ccc(-c4ccccc4)cc3)n2)c1]

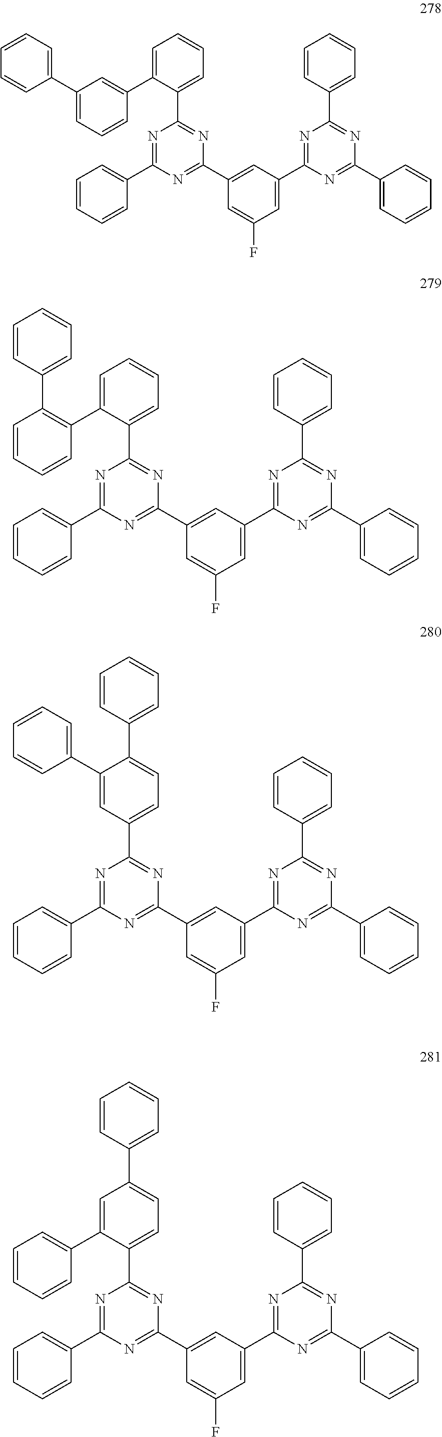 [SMILES: Fc1cc(-c2nc(-c3ccccc3)nc(-c3ccccc3)n2)cc(-c2nc(-c3ccccc3)nc(-c3ccccc3-c3cccc(-c4ccccc4)c3)n2)c1 ; Fc1cc(-c2nc(-c3ccccc3)nc(-c3ccccc3)n2)cc(-c2nc(-c3ccccc3)nc(-c3ccccc3-c3ccccc3-c3ccccc3)n2)c1 ; Fc1cc(-c2nc(-c3ccccc3)nc(-c3ccccc3)n2)cc(-c2nc(-c3ccccc3)nc(-c3ccc(-c4ccccc4)c(-c4ccccc4)c3)n2)c1 ; Fc1cc(-c2nc(-c3ccccc3)nc(-c3ccccc3)n2)cc(-c2nc(-c3ccccc3)nc(-c3ccc(-c4ccccc4)cc3-c3ccccc3)n2)c1]

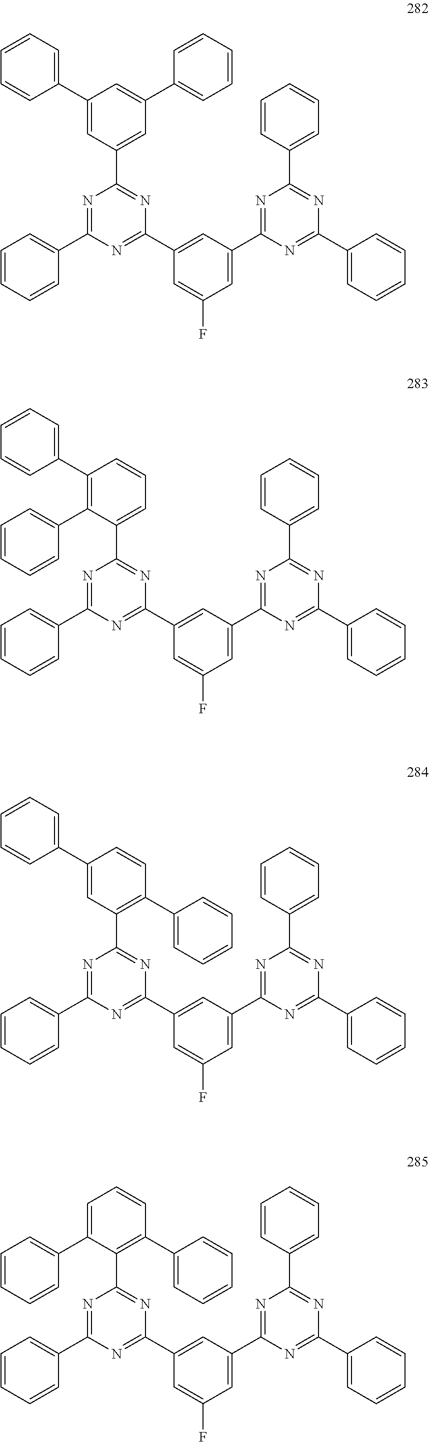 [SMILES: Fc1cc(-c2nc(-c3ccccc3)nc(-c3ccccc3)n2)cc(-c2nc(-c3ccccc3)nc(-c3cc(-c4ccccc4)cc(-c4ccccc4)c3)n2)c1 ; Fc1cc(-c2nc(-c3ccccc3)nc(-c3ccccc3)n2)cc(-c2nc(-c3ccccc3)nc(-c3cccc(-c4ccccc4)c3-c3ccccc3)n2)c1 ; Fc1cc(-c2nc(-c3ccccc3)nc(-c3ccccc3)n2)cc(-c2nc(-c3ccccc3)nc(-c3cc(-c4ccccc4)ccc3-c3ccccc3)n2)c1 ; Fc1cc(-c2nc(-c3ccccc3)nc(-c3ccccc3)n2)cc(-c2nc(-c3ccccc3)nc(-c3c(-c4ccccc4)cccc3-c3ccccc3)n2)c1]

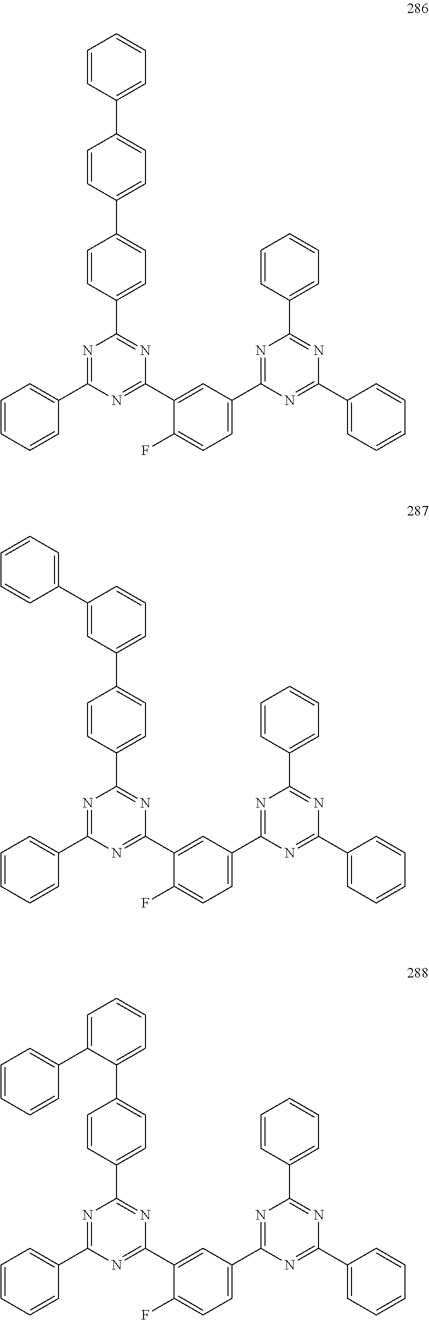 [SMILES: Fc1ccc(-c2nc(-c3ccccc3)nc(-c3ccccc3)n2)cc1-c1nc(-c2ccccc2)nc(-c2ccc(-c3ccc(-c4ccccc4)cc3)cc2)n1 ; Fc1ccc(-c2nc(-c3ccccc3)nc(-c3ccccc3)n2)cc1-c1nc(-c2ccccc2)nc(-c2ccc(-c3cccc(-c4ccccc4)c3)cc2)n1 ; Fc1ccc(-c2nc(-c3ccccc3)nc(-c3ccccc3)n2)cc1-c1nc(-c2ccccc2)nc(-c2ccc(-c3ccccc3-c3ccccc3)cc2)n1]

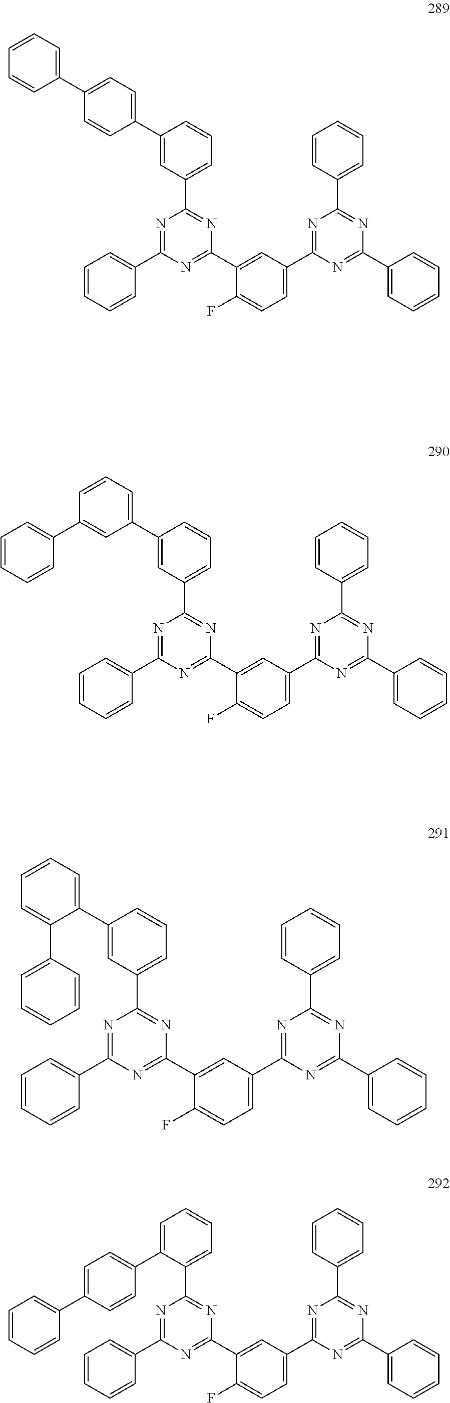 [SMILES: Fc1ccc(-c2nc(-c3ccccc3)nc(-c3ccccc3)n2)cc1-c1nc(-c2ccccc2)nc(-c2cccc(-c3ccc(-c4ccccc4)cc3)c2)n1 ; Fc1ccc(-c2nc(-c3ccccc3)nc(-c3ccccc3)n2)cc1-c1nc(-c2ccccc2)nc(-c2cccc(-c3cccc(-c4ccccc4)c3)c2)n1 ; Fc1ccc(-c2nc(-c3ccccc3)nc(-c3ccccc3)n2)cc1-c1nc(-c2ccccc2)nc(-c2cccc(-c3ccccc3-c3ccccc3)c2)n1 ; Fc1ccc(-c2nc(-c3ccccc3)nc(-c3ccccc3)n2)cc1-c1nc(-c2ccccc2)nc(-c2ccccc2-c2ccc(-c3ccccc3)cc2)n1]

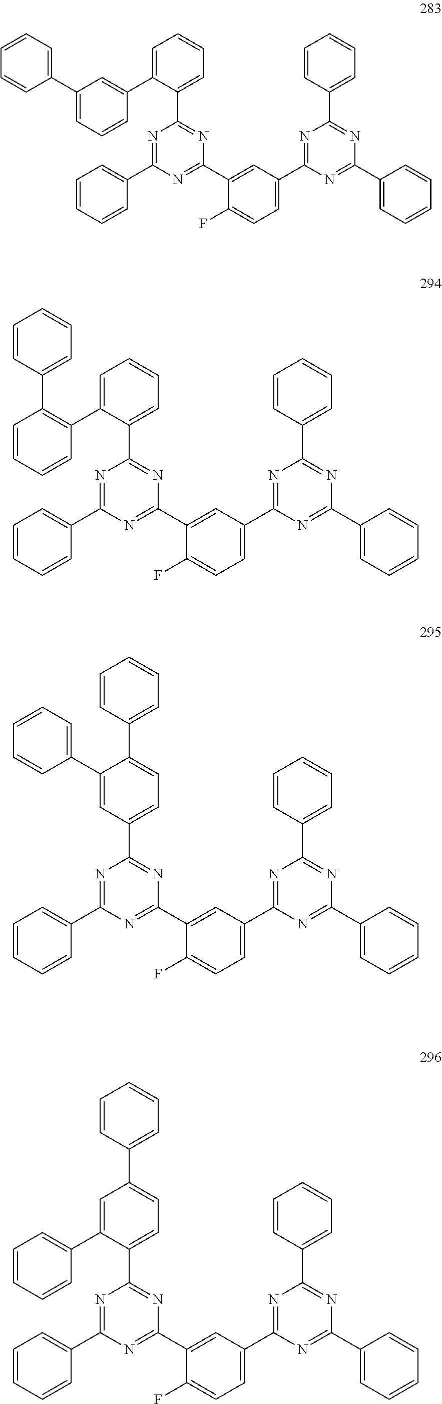 [SMILES: Fc1ccc(-c2nc(-c3ccccc3)nc(-c3ccccc3)n2)cc1-c1nc(-c2ccccc2)nc(-c2ccccc2-c2cccc(-c3ccccc3)c2)n1 ; Fc1ccc(-c2nc(-c3ccccc3)nc(-c3ccccc3)n2)cc1-c1nc(-c2ccccc2)nc(-c2ccccc2-c2ccccc2-c2ccccc2)n1 ; Fc1ccc(-c2nc(-c3ccccc3)nc(-c3ccccc3)n2)cc1-c1nc(-c2ccccc2)nc(-c2ccc(-c3ccccc3)c(-c3ccccc3)c2)n1 ; Fc1ccc(-c2nc(-c3ccccc3)nc(-c3ccccc3)n2)cc1-c1nc(-c2ccccc2)nc(-c2ccc(-c3ccccc3)cc2-c2ccccc2)n1]

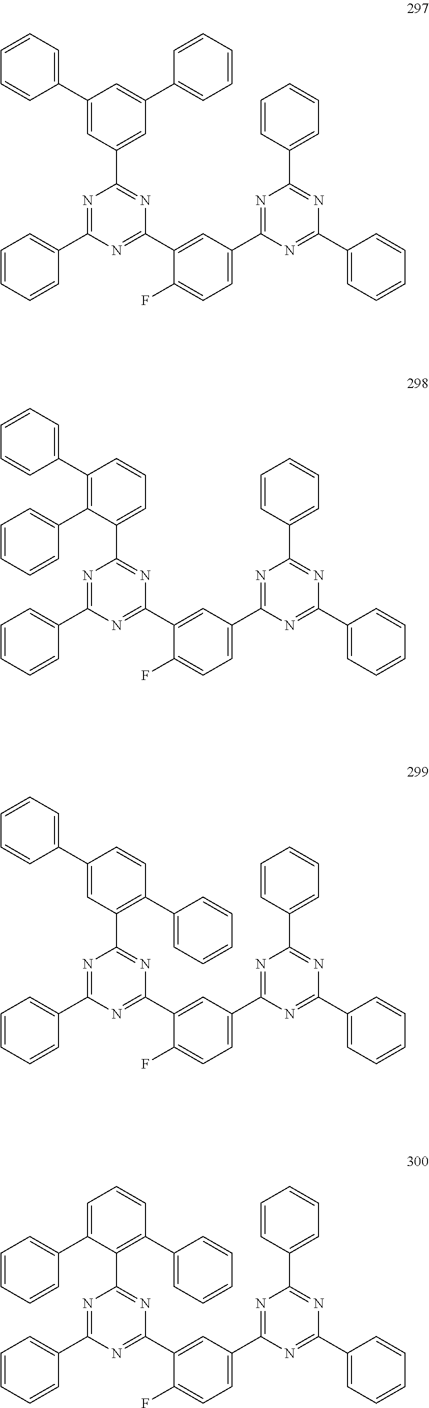 [SMILES: Fc1ccc(-c2nc(-c3ccccc3)nc(-c3ccccc3)n2)cc1-c1nc(-c2ccccc2)nc(-c2cc(-c3ccccc3)cc(-c3ccccc3)c2)n1 ; Fc1ccc(-c2nc(-c3ccccc3)nc(-c3ccccc3)n2)cc1-c1nc(-c2ccccc2)nc(-c2cccc(-c3ccccc3)c2-c2ccccc2)n1 ; Fc1ccc(-c2nc(-c3ccccc3)nc(-c3ccccc3)n2)cc1-c1nc(-c2ccccc2)nc(-c2cc(-c3ccccc3)ccc2-c2ccccc2)n1 ; Fc1ccc(-c2nc(-c3ccccc3)nc(-c3ccccc3)n2)cc1-c1nc(-c2ccccc2)nc(-c2c(-c3ccccc3)cccc2-c2ccccc2)n1]

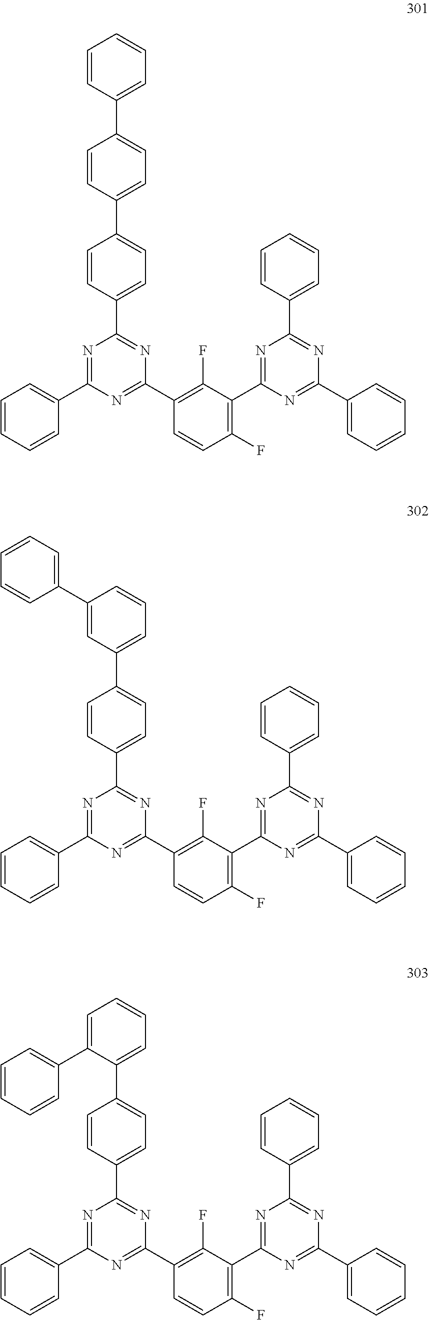 [SMILES: Fc1ccc(-c2nc(-c3ccccc3)nc(-c3ccc(-c4ccc(-c5ccccc5)cc4)cc3)n2)c(F)c1-c1nc(-c2ccccc2)nc(-c2ccccc2)n1 ; Fc1ccc(-c2nc(-c3ccccc3)nc(-c3ccc(-c4cccc(-c5ccccc5)c4)cc3)n2)c(F)c1-c1nc(-c2ccccc2)nc(-c2ccccc2)n1 ; Fc1ccc(-c2nc(-c3ccccc3)nc(-c3ccc(-c4ccccc4-c4ccccc4)cc3)n2)c(F)c1-c1nc(-c2ccccc2)nc(-c2ccccc2)n1]

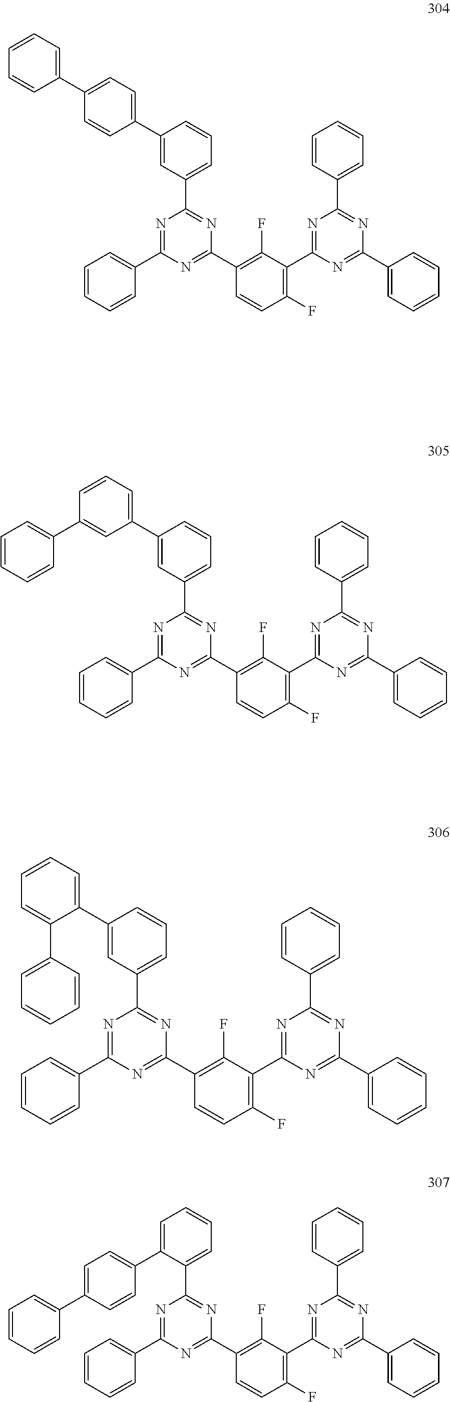 [SMILES: Fc1ccc(-c2nc(-c3ccccc3)nc(-c3cccc(-c4ccc(-c5ccccc5)cc4)c3)n2)c(F)c1-c1nc(-c2ccccc2)nc(-c2ccccc2)n1 ; Fc1ccc(-c2nc(-c3ccccc3)nc(-c3cccc(-c4cccc(-c5ccccc5)c4)c3)n2)c(F)c1-c1nc(-c2ccccc2)nc(-c2ccccc2)n1 ; Fc1ccc(-c2nc(-c3ccccc3)nc(-c3cccc(-c4ccccc4-c4ccccc4)c3)n2)c(F)c1-c1nc(-c2ccccc2)nc(-c2ccccc2)n1 ; Fc1ccc(-c2nc(-c3ccccc3)nc(-c3ccccc3-c3ccc(-c4ccccc4)cc3)n2)c(F)c1-c1nc(-c2ccccc2)nc(-c2ccccc2)n1]

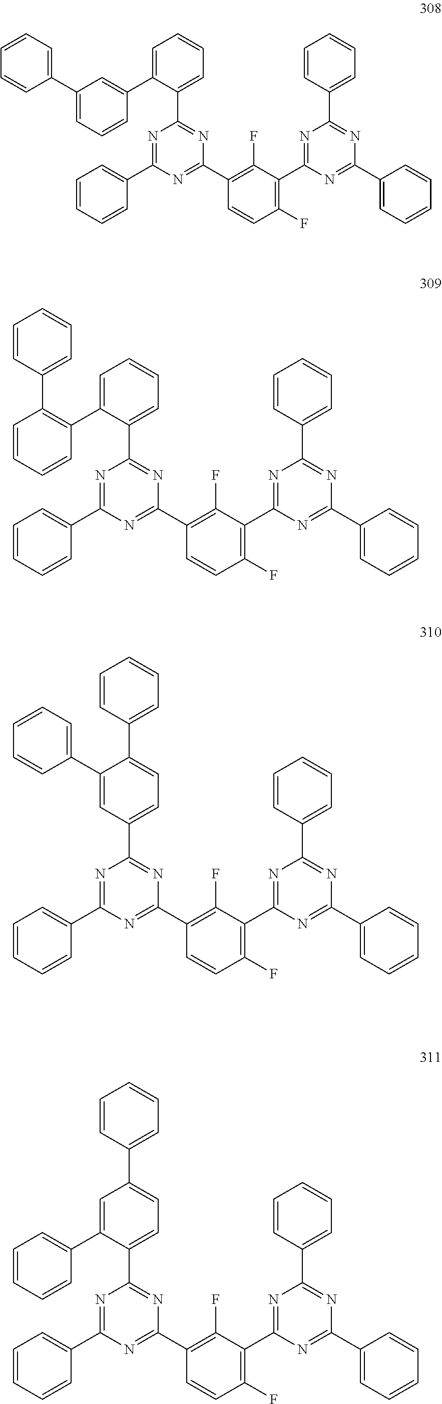 [SMILES: Fc1ccc(-c2nc(-c3ccccc3)nc(-c3ccccc3-c3cccc(-c4ccccc4)c3)n2)c(F)c1-c1nc(-c2ccccc2)nc(-c2ccccc2)n1 ; Fc1ccc(-c2nc(-c3ccccc3)nc(-c3ccccc3-c3ccccc3-c3ccccc3)n2)c(F)c1-c1nc(-c2ccccc2)nc(-c2ccccc2)n1 ; Fc1ccc(-c2nc(-c3ccccc3)nc(-c3ccc(-c4ccccc4)c(-c4ccccc4)c3)n2)c(F)c1-c1nc(-c2ccccc2)nc(-c2ccccc2)n1 ; Fc1ccc(-c2nc(-c3ccccc3)nc(-c3ccc(-c4ccccc4)cc3-c3ccccc3)n2)c(F)c1-c1nc(-c2ccccc2)nc(-c2ccccc2)n1]

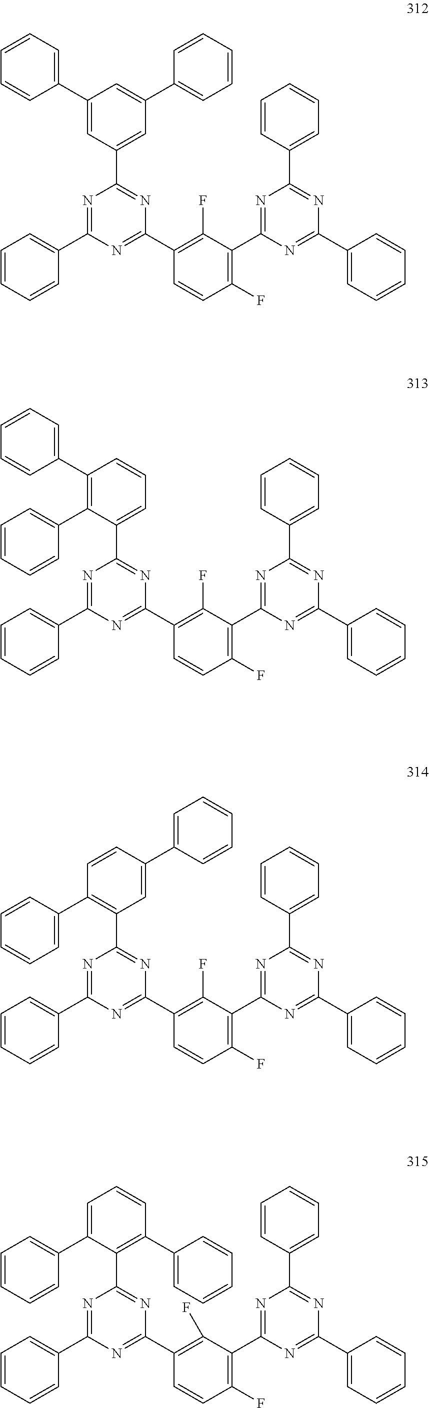 [SMILES: Fc1ccc(-c2nc(-c3ccccc3)nc(-c3cc(-c4ccccc4)cc(-c4ccccc4)c3)n2)c(F)c1-c1nc(-c2ccccc2)nc(-c2ccccc2)n1 ; Fc1ccc(-c2nc(-c3ccccc3)nc(-c3cccc(-c4ccccc4)c3-c3ccccc3)n2)c(F)c1-c1nc(-c2ccccc2)nc(-c2ccccc2)n1 ; Fc1ccc(-c2nc(-c3ccccc3)nc(-c3cc(-c4ccccc4)ccc3-c3ccccc3)n2)c(F)c1-c1nc(-c2ccccc2)nc(-c2ccccc2)n1 ; Fc1ccc(-c2nc(-c3ccccc3)nc(-c3c(-c4ccccc4)cccc3-c3ccccc3)n2)c(F)c1-c1nc(-c2ccccc2)nc(-c2ccccc2)n1]

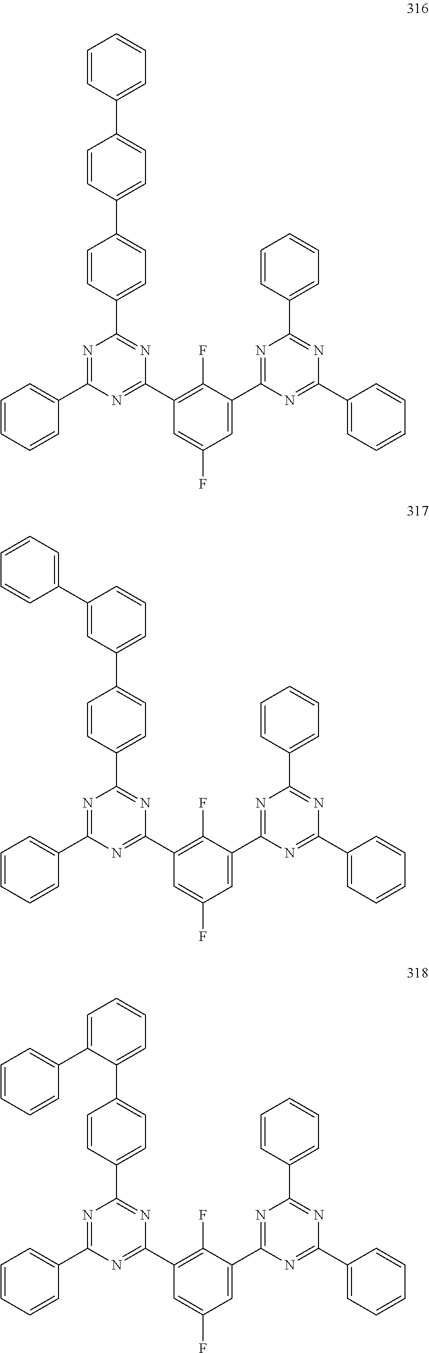 [SMILES: Fc1cc(-c2nc(-c3ccccc3)nc(-c3ccccc3)n2)c(F)c(-c2nc(-c3ccccc3)nc(-c3ccc(-c4ccc(-c5ccccc5)cc4)cc3)n2)c1 ; Fc1cc(-c2nc(-c3ccccc3)nc(-c3ccccc3)n2)c(F)c(-c2nc(-c3ccccc3)nc(-c3ccc(-c4cccc(-c5ccccc5)c4)cc3)n2)c1 ; Fc1cc(-c2nc(-c3ccccc3)nc(-c3ccccc3)n2)c(F)c(-c2nc(-c3ccccc3)nc(-c3ccc(-c4ccccc4-c4ccccc4)cc3)n2)c1]

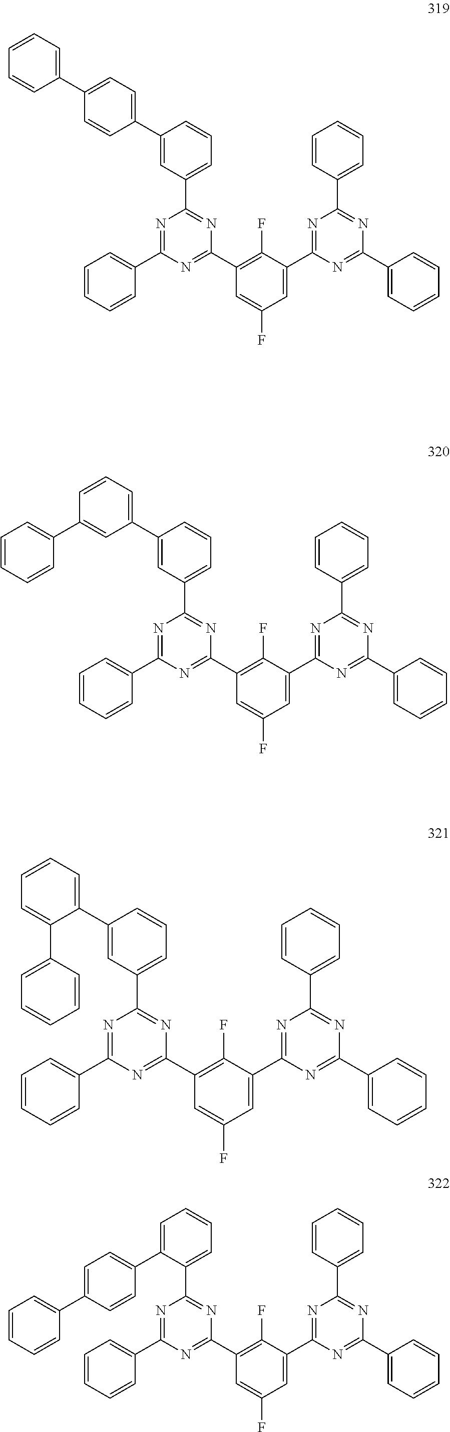 [SMILES: Fc1cc(-c2nc(-c3ccccc3)nc(-c3ccccc3)n2)c(F)c(-c2nc(-c3ccccc3)nc(-c3cccc(-c4ccc(-c5ccccc5)cc4)c3)n2)c1 ; Fc1cc(-c2nc(-c3ccccc3)nc(-c3ccccc3)n2)c(F)c(-c2nc(-c3ccccc3)nc(-c3cccc(-c4cccc(-c5ccccc5)c4)c3)n2)c1 ; Fc1cc(-c2nc(-c3ccccc3)nc(-c3ccccc3)n2)c(F)c(-c2nc(-c3ccccc3)nc(-c3cccc(-c4ccccc4-c4ccccc4)c3)n2)c1 ; Fc1cc(-c2nc(-c3ccccc3)nc(-c3ccccc3)n2)c(F)c(-c2nc(-c3ccccc3)nc(-c3ccccc3-c3ccc(-c4ccccc4)cc3)n2)c1]

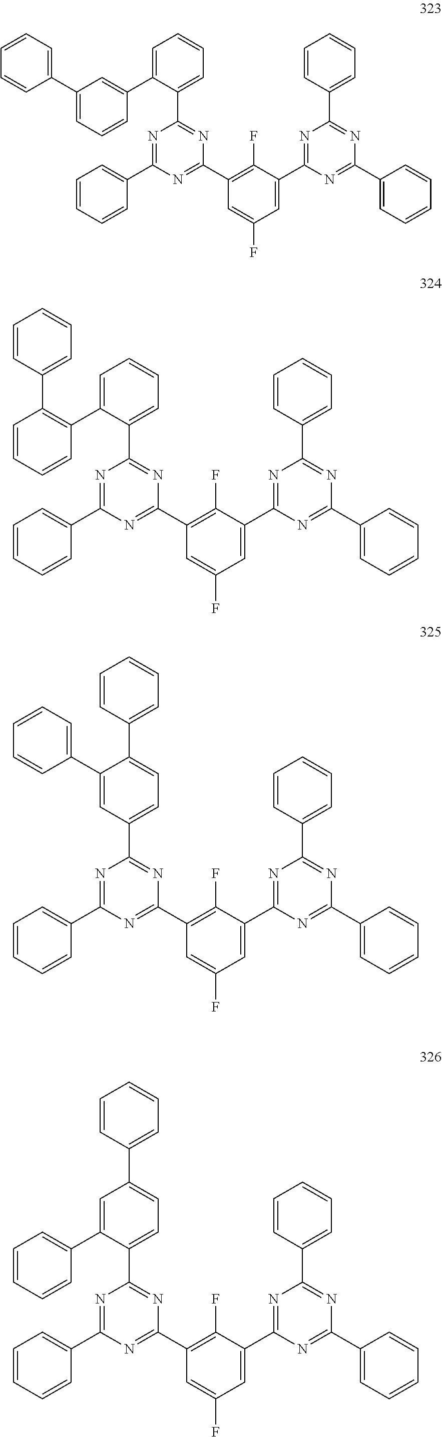 [SMILES: Fc1cc(-c2nc(-c3ccccc3)nc(-c3ccccc3)n2)c(F)c(-c2nc(-c3ccccc3)nc(-c3ccccc3-c3cccc(-c4ccccc4)c3)n2)c1 ; Fc1cc(-c2nc(-c3ccccc3)nc(-c3ccccc3)n2)c(F)c(-c2nc(-c3ccccc3)nc(-c3ccccc3-c3ccccc3-c3ccccc3)n2)c1 ; Fc1cc(-c2nc(-c3ccccc3)nc(-c3ccccc3)n2)c(F)c(-c2nc(-c3ccccc3)nc(-c3ccc(-c4ccccc4)c(-c4ccccc4)c3)n2)c1 ; Fc1cc(-c2nc(-c3ccccc3)nc(-c3ccccc3)n2)c(F)c(-c2nc(-c3ccccc3)nc(-c3ccc(-c4ccccc4)cc3-c3ccccc3)n2)c1]

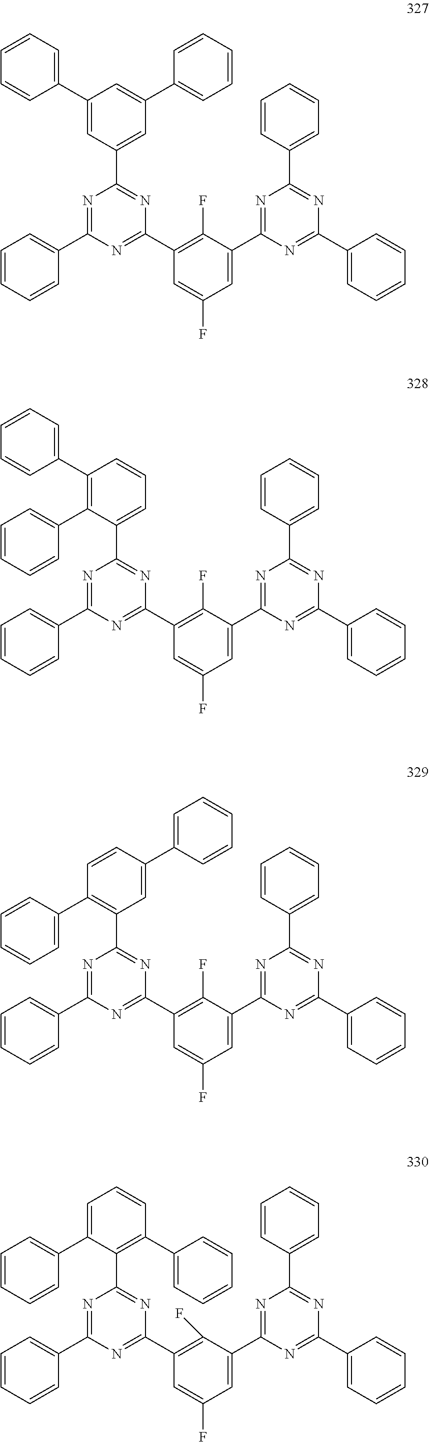 [SMILES: Fc1cc(-c2nc(-c3ccccc3)nc(-c3ccccc3)n2)c(F)c(-c2nc(-c3ccccc3)nc(-c3cc(-c4ccccc4)cc(-c4ccccc4)c3)n2)c1 ; Fc1cc(-c2nc(-c3ccccc3)nc(-c3ccccc3)n2)c(F)c(-c2nc(-c3ccccc3)nc(-c3cccc(-c4ccccc4)c3-c3ccccc3)n2)c1 ; Fc1cc(-c2nc(-c3ccccc3)nc(-c3ccccc3)n2)c(F)c(-c2nc(-c3ccccc3)nc(-c3cc(-c4ccccc4)ccc3-c3ccccc3)n2)c1 ; Fc1cc(-c2nc(-c3ccccc3)nc(-c3ccccc3)n2)c(F)c(-c2nc(-c3ccccc3)nc(-c3c(-c4ccccc4)cccc3-c3ccccc3)n2)c1]

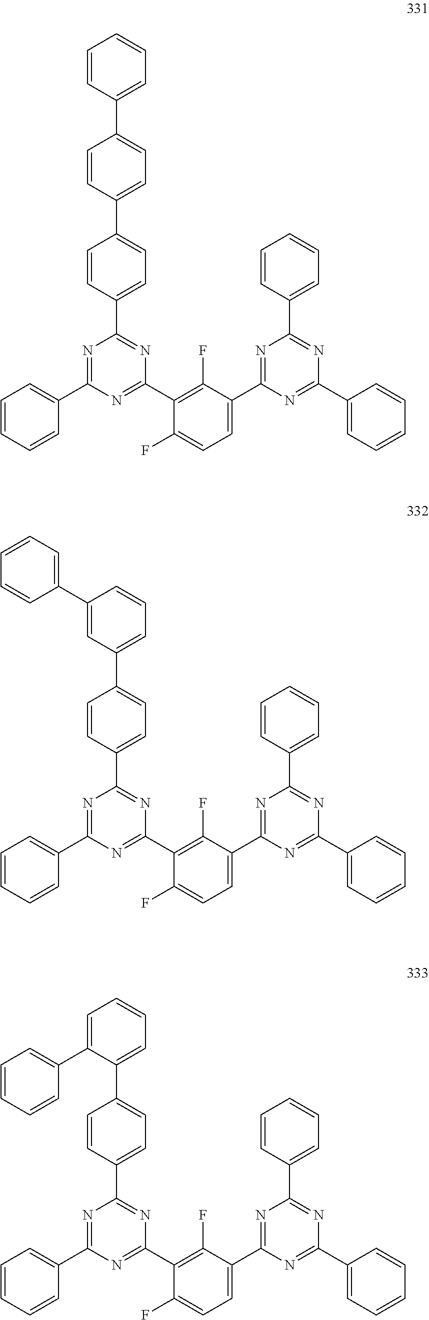 [SMILES: Fc1ccc(-c2nc(-c3ccccc3)nc(-c3ccccc3)n2)c(F)c1-c1nc(-c2ccccc2)nc(-c2ccc(-c3ccc(-c4ccccc4)cc3)cc2)n1 ; Fc1ccc(-c2nc(-c3ccccc3)nc(-c3ccccc3)n2)c(F)c1-c1nc(-c2ccccc2)nc(-c2ccc(-c3cccc(-c4ccccc4)c3)cc2)n1 ; Fc1ccc(-c2nc(-c3ccccc3)nc(-c3ccccc3)n2)c(F)c1-c1nc(-c2ccccc2)nc(-c2ccc(-c3ccccc3-c3ccccc3)cc2)n1]

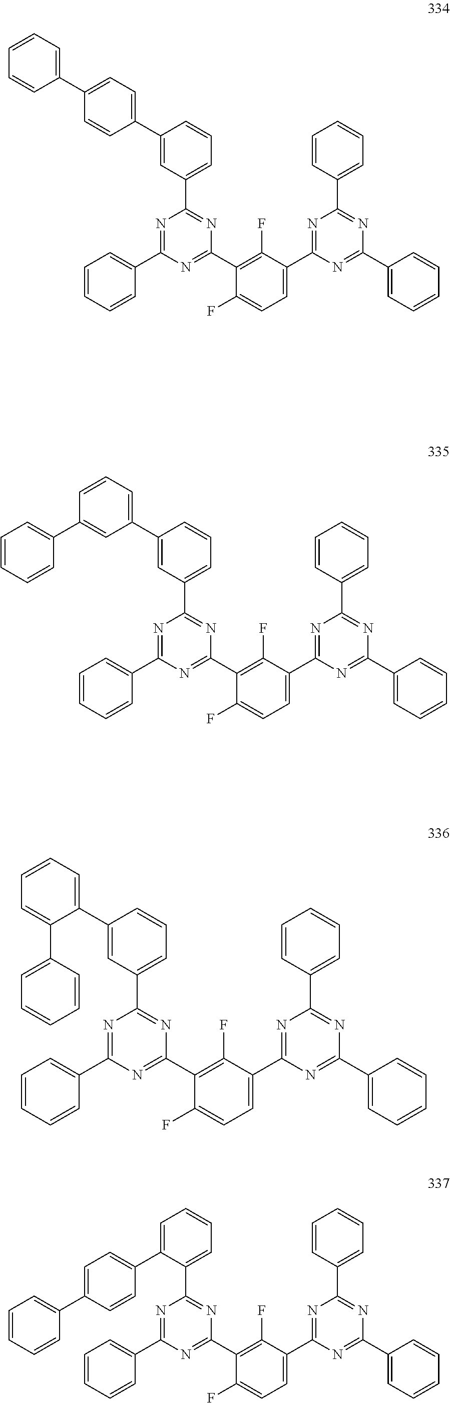 [SMILES: Fc1ccc(-c2nc(-c3ccccc3)nc(-c3ccccc3)n2)c(F)c1-c1nc(-c2ccccc2)nc(-c2cccc(-c3ccc(-c4ccccc4)cc3)c2)n1 ; Fc1ccc(-c2nc(-c3ccccc3)nc(-c3ccccc3)n2)c(F)c1-c1nc(-c2ccccc2)nc(-c2cccc(-c3cccc(-c4ccccc4)c3)c2)n1 ; Fc1ccc(-c2nc(-c3ccccc3)nc(-c3ccccc3)n2)c(F)c1-c1nc(-c2ccccc2)nc(-c2cccc(-c3ccccc3-c3ccccc3)c2)n1 ; Fc1ccc(-c2nc(-c3ccccc3)nc(-c3ccccc3)n2)c(F)c1-c1nc(-c2ccccc2)nc(-c2ccccc2-c2ccc(-c3ccccc3)cc2)n1]

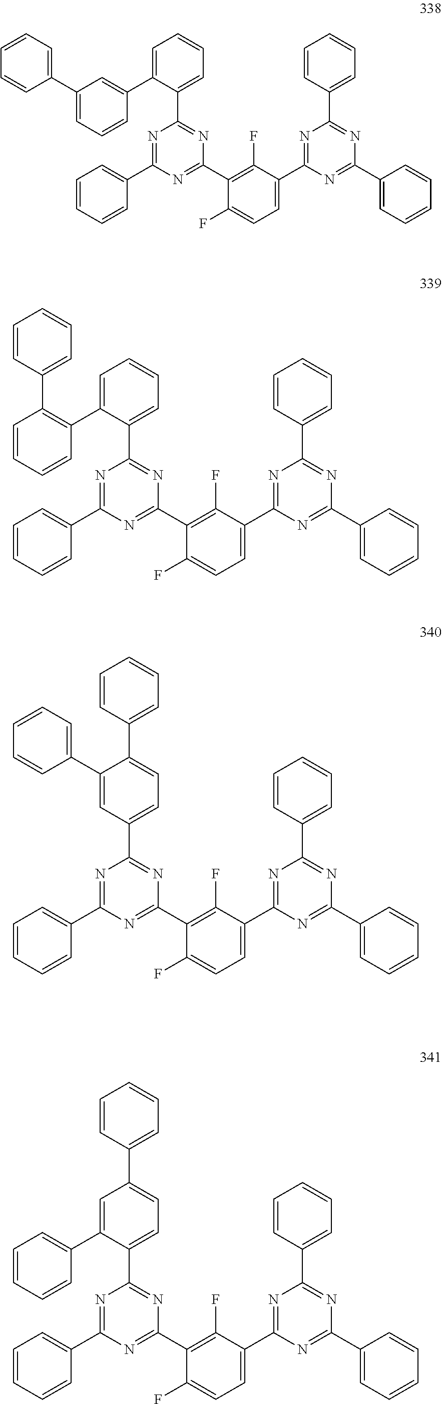 [SMILES: Fc1ccc(-c2nc(-c3ccccc3)nc(-c3ccccc3)n2)c(F)c1-c1nc(-c2ccccc2)nc(-c2ccccc2-c2cccc(-c3ccccc3)c2)n1 ; Fc1ccc(-c2nc(-c3ccccc3)nc(-c3ccccc3)n2)c(F)c1-c1nc(-c2ccccc2)nc(-c2ccccc2-c2ccccc2-c2ccccc2)n1 ; Fc1ccc(-c2nc(-c3ccccc3)nc(-c3ccccc3)n2)c(F)c1-c1nc(-c2ccccc2)nc(-c2ccc(-c3ccccc3)c(-c3ccccc3)c2)n1 ; Fc1ccc(-c2nc(-c3ccccc3)nc(-c3ccccc3)n2)c(F)c1-c1nc(-c2ccccc2)nc(-c2ccc(-c3ccccc3)cc2-c2ccccc2)n1]

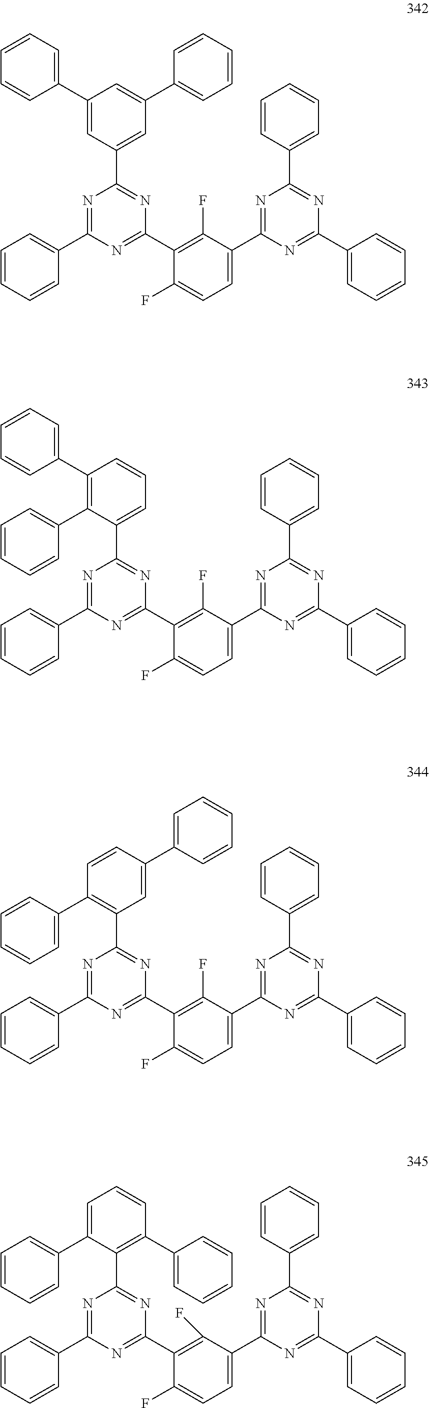 [SMILES: Fc1ccc(-c2nc(-c3ccccc3)nc(-c3ccccc3)n2)c(F)c1-c1nc(-c2ccccc2)nc(-c2cc(-c3ccccc3)cc(-c3ccccc3)c2)n1 ; Fc1ccc(-c2nc(-c3ccccc3)nc(-c3ccccc3)n2)c(F)c1-c1nc(-c2ccccc2)nc(-c2cccc(-c3ccccc3)c2-c2ccccc2)n1 ; Fc1ccc(-c2nc(-c3ccccc3)nc(-c3ccccc3)n2)c(F)c1-c1nc(-c2ccccc2)nc(-c2cc(-c3ccccc3)ccc2-c2ccccc2)n1 ; Fc1ccc(-c2nc(-c3ccccc3)nc(-c3ccccc3)n2)c(F)c1-c1nc(-c2ccccc2)nc(-c2c(-c3ccccc3)cccc2-c2ccccc2)n1]

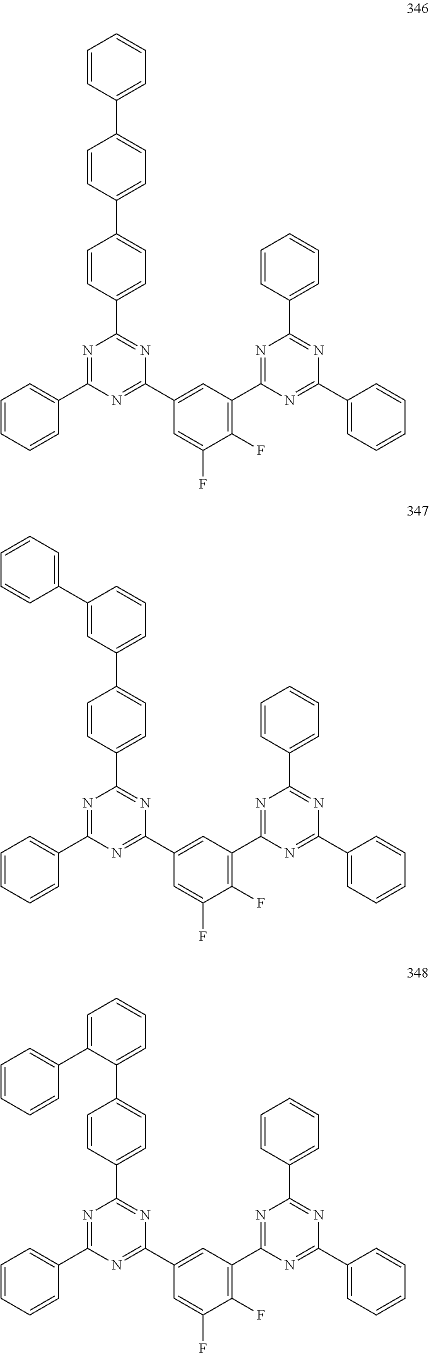 [SMILES: Fc1cc(-c2nc(-c3ccccc3)nc(-c3ccc(-c4ccc(-c5ccccc5)cc4)cc3)n2)cc(-c2nc(-c3ccccc3)nc(-c3ccccc3)n2)c1F ; Fc1cc(-c2nc(-c3ccccc3)nc(-c3ccc(-c4cccc(-c5ccccc5)c4)cc3)n2)cc(-c2nc(-c3ccccc3)nc(-c3ccccc3)n2)c1F ; Fc1cc(-c2nc(-c3ccccc3)nc(-c3ccc(-c4ccccc4-c4ccccc4)cc3)n2)cc(-c2nc(-c3ccccc3)nc(-c3ccccc3)n2)c1F]

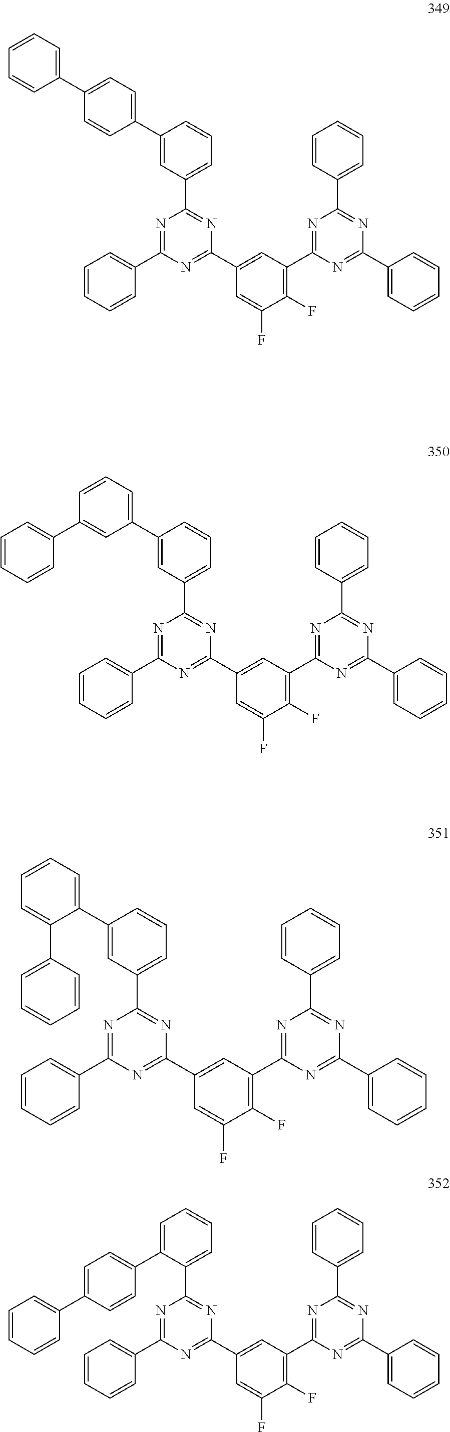 [SMILES: Fc1cc(-c2nc(-c3ccccc3)nc(-c3cccc(-c4ccc(-c5ccccc5)cc4)c3)n2)cc(-c2nc(-c3ccccc3)nc(-c3ccccc3)n2)c1F ; Fc1cc(-c2nc(-c3ccccc3)nc(-c3cccc(-c4cccc(-c5ccccc5)c4)c3)n2)cc(-c2nc(-c3ccccc3)nc(-c3ccccc3)n2)c1F ; Fc1cc(-c2nc(-c3ccccc3)nc(-c3cccc(-c4ccccc4-c4ccccc4)c3)n2)cc(-c2nc(-c3ccccc3)nc(-c3ccccc3)n2)c1F ; Fc1cc(-c2nc(-c3ccccc3)nc(-c3ccccc3-c3ccc(-c4ccccc4)cc3)n2)cc(-c2nc(-c3ccccc3)nc(-c3ccccc3)n2)c1F]

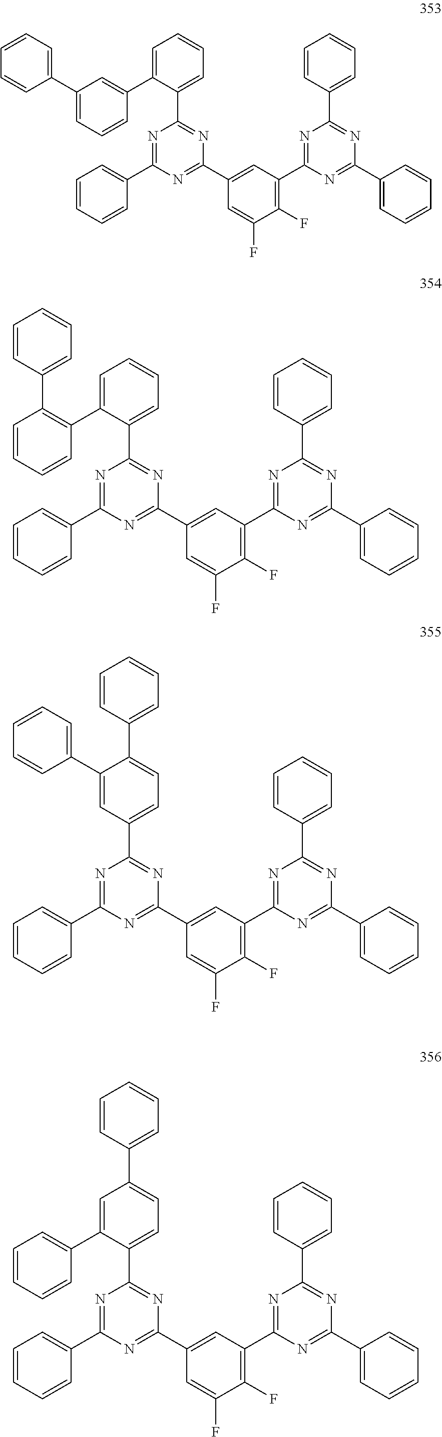 [SMILES: Fc1cc(-c2nc(-c3ccccc3)nc(-c3ccccc3-c3cccc(-c4ccccc4)c3)n2)cc(-c2nc(-c3ccccc3)nc(-c3ccccc3)n2)c1F ; Fc1cc(-c2nc(-c3ccccc3)nc(-c3ccccc3-c3ccccc3-c3ccccc3)n2)cc(-c2nc(-c3ccccc3)nc(-c3ccccc3)n2)c1F ; Fc1cc(-c2nc(-c3ccccc3)nc(-c3ccc(-c4ccccc4)c(-c4ccccc4)c3)n2)cc(-c2nc(-c3ccccc3)nc(-c3ccccc3)n2)c1F ; Fc1cc(-c2nc(-c3ccccc3)nc(-c3ccc(-c4ccccc4)cc3-c3ccccc3)n2)cc(-c2nc(-c3ccccc3)nc(-c3ccccc3)n2)c1F]

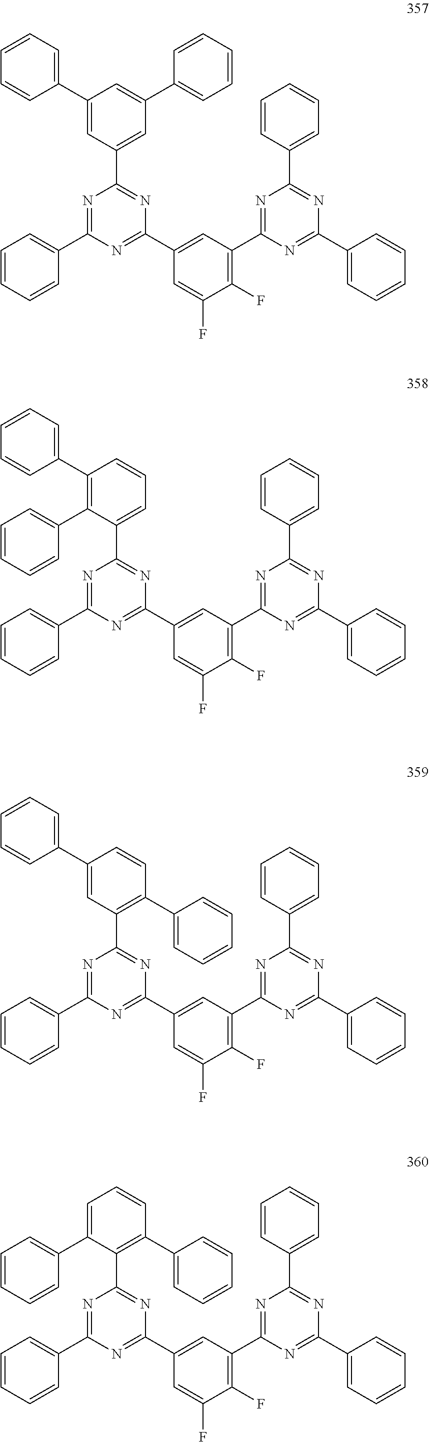 [SMILES: Fc1cc(-c2nc(-c3ccccc3)nc(-c3cc(-c4ccccc4)cc(-c4ccccc4)c3)n2)cc(-c2nc(-c3ccccc3)nc(-c3ccccc3)n2)c1F ; Fc1cc(-c2nc(-c3ccccc3)nc(-c3cccc(-c4ccccc4)c3-c3ccccc3)n2)cc(-c2nc(-c3ccccc3)nc(-c3ccccc3)n2)c1F ; Fc1cc(-c2nc(-c3ccccc3)nc(-c3cc(-c4ccccc4)ccc3-c3ccccc3)n2)cc(-c2nc(-c3ccccc3)nc(-c3ccccc3)n2)c1F ; Fc1cc(-c2nc(-c3ccccc3)nc(-c3c(-c4ccccc4)cccc3-c3ccccc3)n2)cc(-c2nc(-c3ccccc3)nc(-c3ccccc3)n2)c1F]

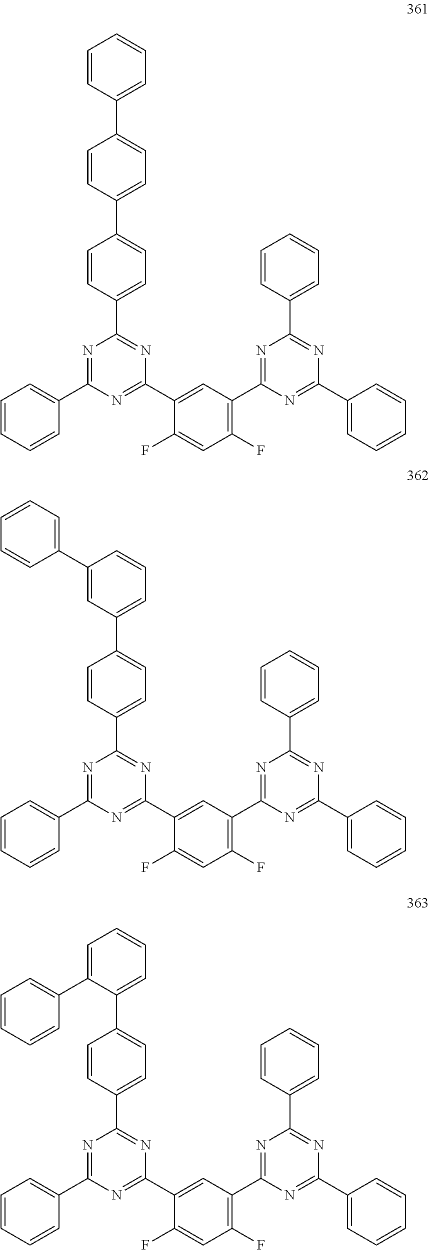 [SMILES: Fc1cc(F)c(-c2nc(-c3ccccc3)nc(-c3ccc(-c4ccc(-c5ccccc5)cc4)cc3)n2)cc1-c1nc(-c2ccccc2)nc(-c2ccccc2)n1 ; Fc1cc(F)c(-c2nc(-c3ccccc3)nc(-c3ccc(-c4cccc(-c5ccccc5)c4)cc3)n2)cc1-c1nc(-c2ccccc2)nc(-c2ccccc2)n1 ; Fc1cc(F)c(-c2nc(-c3ccccc3)nc(-c3ccc(-c4ccccc4-c4ccccc4)cc3)n2)cc1-c1nc(-c2ccccc2)nc(-c2ccccc2)n1]

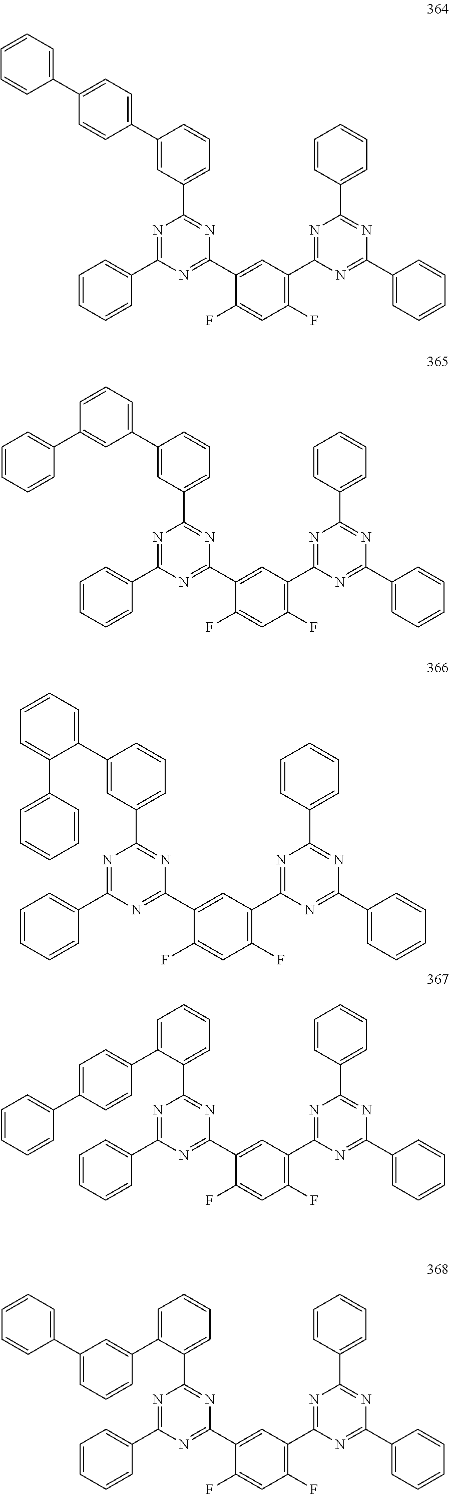 [SMILES: Fc1cc(F)c(-c2nc(-c3ccccc3)nc(-c3cccc(-c4ccc(-c5ccccc5)cc4)c3)n2)cc1-c1nc(-c2ccccc2)nc(-c2ccccc2)n1 ; Fc1cc(F)c(-c2nc(-c3ccccc3)nc(-c3cccc(-c4cccc(-c5ccccc5)c4)c3)n2)cc1-c1nc(-c2ccccc2)nc(-c2ccccc2)n1 ; Fc1cc(F)c(-c2nc(-c3ccccc3)nc(-c3cccc(-c4ccccc4-c4ccccc4)c3)n2)cc1-c1nc(-c2ccccc2)nc(-c2ccccc2)n1 ; Fc1cc(F)c(-c2nc(-c3ccccc3)nc(-c3ccccc3-c3ccc(-c4ccccc4)cc3)n2)cc1-c1nc(-c2ccccc2)nc(-c2ccccc2)n1 ; Fc1cc(F)c(-c2nc(-c3ccccc3)nc(-c3ccccc3-c3cccc(-c4ccccc4)c3)n2)cc1-c1nc(-c2ccccc2)nc(-c2ccccc2)n1]

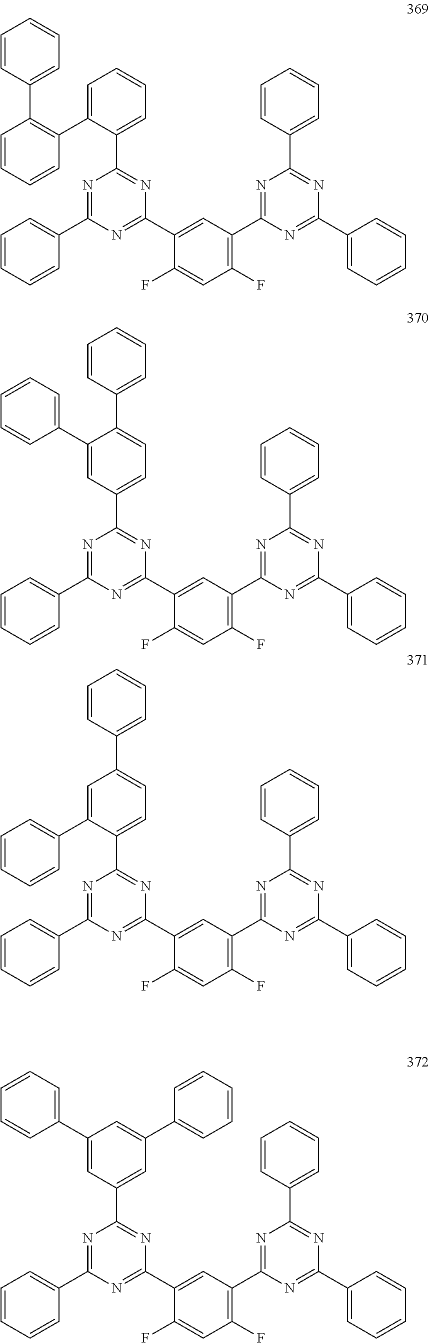

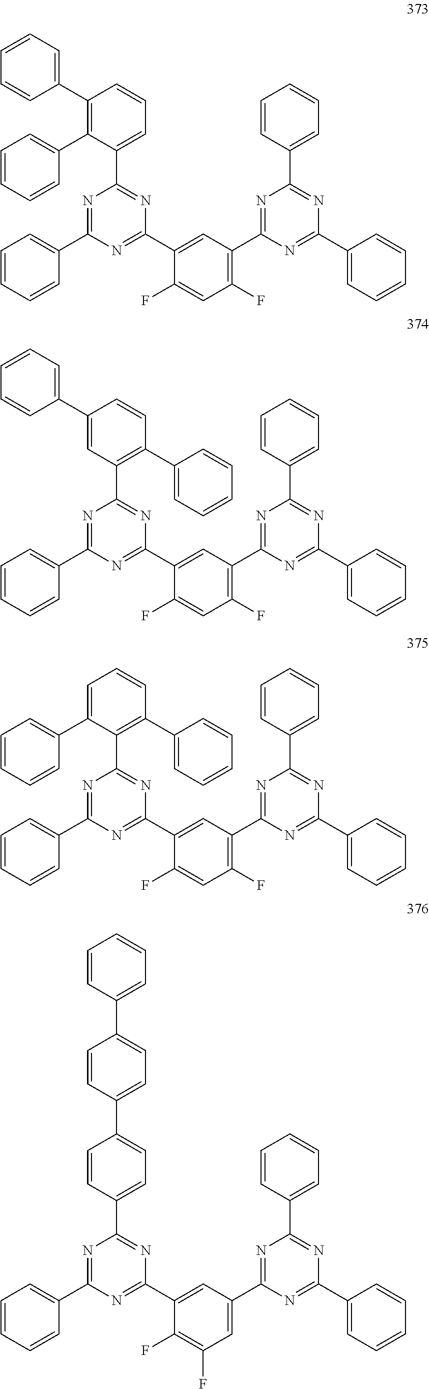 [SMILES: Fc1cc(F)c(-c2nc(-c3ccccc3)nc(-c3cccc(-c4ccccc4)c3-c3ccccc3)n2)cc1-c1nc(-c2ccccc2)nc(-c2ccccc2)n1 ; Fc1cc(F)c(-c2nc(-c3ccccc3)nc(-c3cc(-c4ccccc4)ccc3-c3ccccc3)n2)cc1-c1nc(-c2ccccc2)nc(-c2ccccc2)n1 ; Fc1cc(F)c(-c2nc(-c3ccccc3)nc(-c3c(-c4ccccc4)cccc3-c3ccccc3)n2)cc1-c1nc(-c2ccccc2)nc(-c2ccccc2)n1 ; Fc1cc(-c2nc(-c3ccccc3)nc(-c3ccccc3)n2)cc(-c2nc(-c3ccccc3)nc(-c3ccc(-c4ccc(-c5ccccc5)cc4)cc3)n2)c1F]

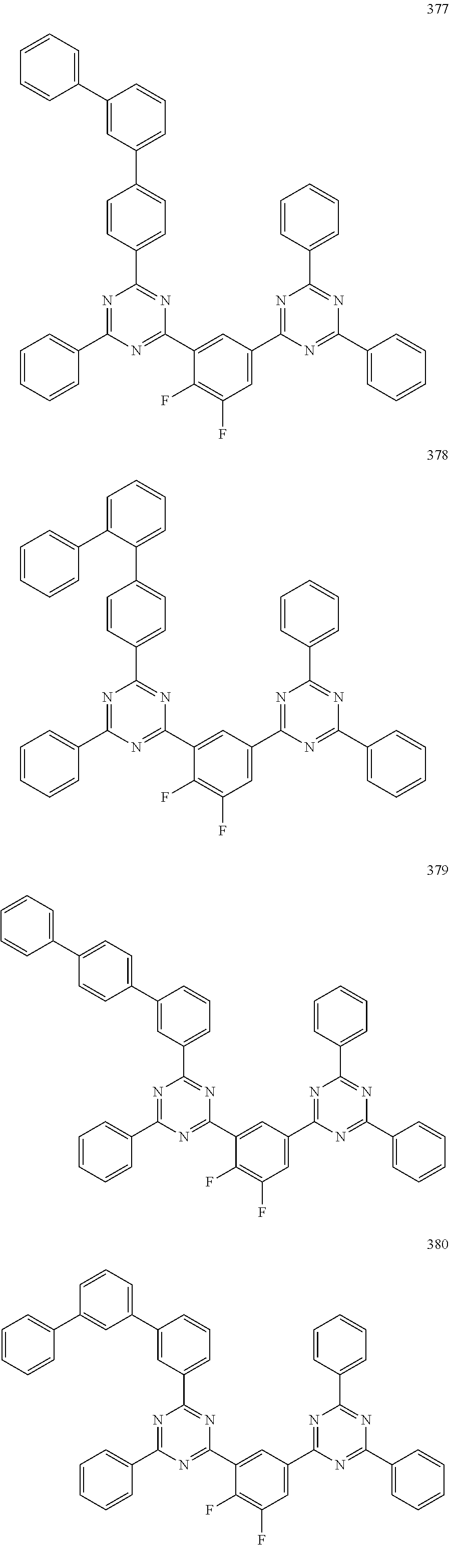 [SMILES: Fc1cc(-c2nc(-c3ccccc3)nc(-c3ccccc3)n2)cc(-c2nc(-c3ccccc3)nc(-c3ccc(-c4cccc(-c5ccccc5)c4)cc3)n2)c1F ; Fc1cc(-c2nc(-c3ccccc3)nc(-c3ccccc3)n2)cc(-c2nc(-c3ccccc3)nc(-c3ccc(-c4ccccc4-c4ccccc4)cc3)n2)c1F ; Fc1cc(-c2nc(-c3ccccc3)nc(-c3ccccc3)n2)cc(-c2nc(-c3ccccc3)nc(-c3cccc(-c4ccc(-c5ccccc5)cc4)c3)n2)c1F ; Fc1cc(-c2nc(-c3ccccc3)nc(-c3ccccc3)n2)cc(-c2nc(-c3ccccc3)nc(-c3cccc(-c4cccc(-c5ccccc5)c4)c3)n2)c1F]

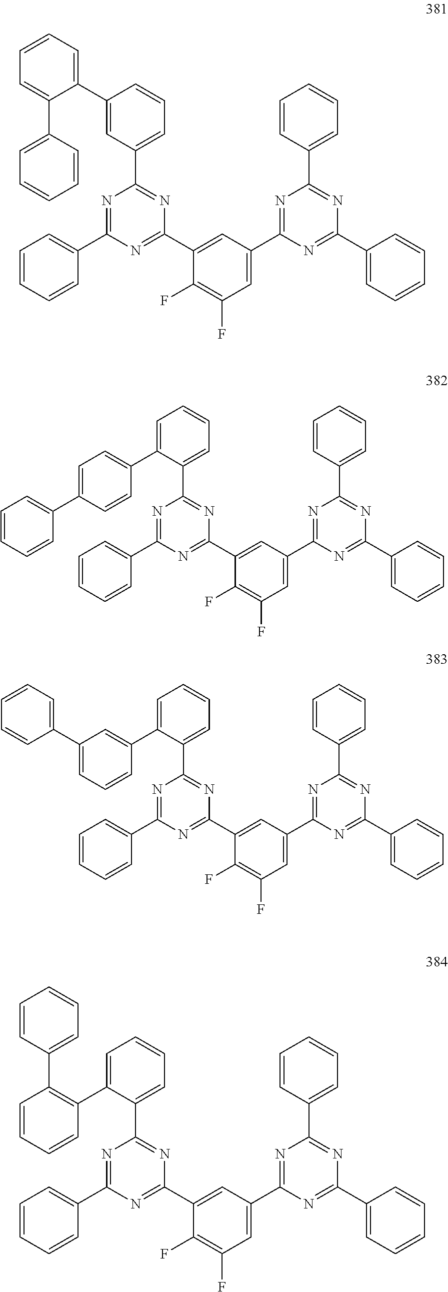 [SMILES: Fc1cc(-c2nc(-c3ccccc3)nc(-c3ccccc3)n2)cc(-c2nc(-c3ccccc3)nc(-c3cccc(-c4ccccc4-c4ccccc4)c3)n2)c1F ; Fc1cc(-c2nc(-c3ccccc3)nc(-c3ccccc3)n2)cc(-c2nc(-c3ccccc3)nc(-c3ccccc3-c3ccc(-c4ccccc4)cc3)n2)c1F ; Fc1cc(-c2nc(-c3ccccc3)nc(-c3ccccc3)n2)cc(-c2nc(-c3ccccc3)nc(-c3ccccc3-c3cccc(-c4ccccc4)c3)n2)c1F ; Fc1cc(-c2nc(-c3ccccc3)nc(-c3ccccc3)n2)cc(-c2nc(-c3ccccc3)nc(-c3ccccc3-c3ccccc3-c3ccccc3)n2)c1F]

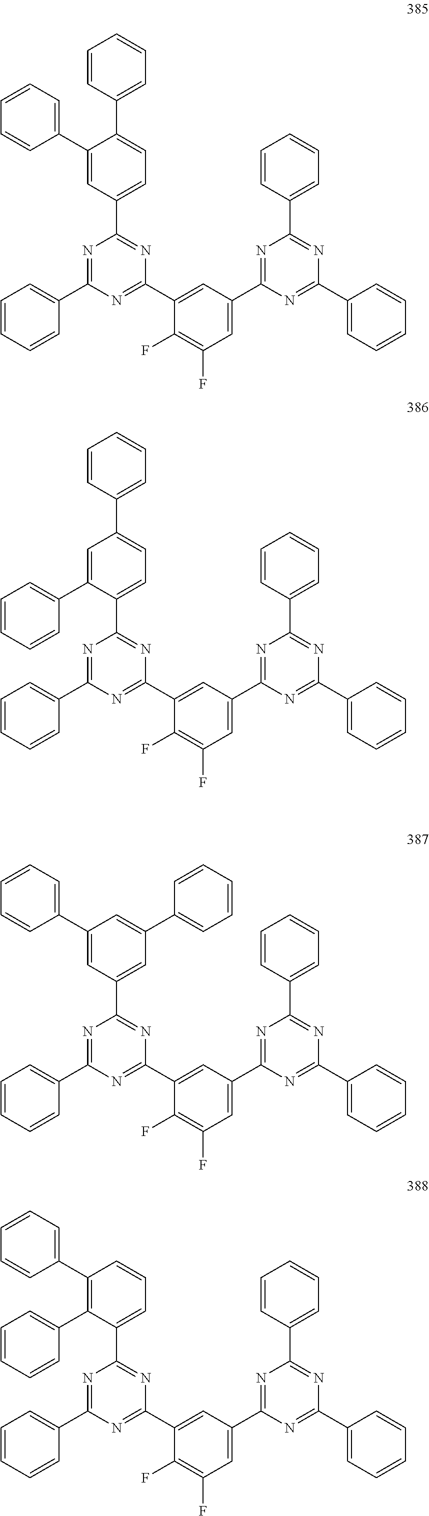 [SMILES: Fc1cc(-c2nc(-c3ccccc3)nc(-c3ccccc3)n2)cc(-c2nc(-c3ccccc3)nc(-c3ccc(-c4ccccc4)c(-c4ccccc4)c3)n2)c1F ; Fc1cc(-c2nc(-c3ccccc3)nc(-c3ccccc3)n2)cc(-c2nc(-c3ccccc3)nc(-c3ccc(-c4ccccc4)cc3-c3ccccc3)n2)c1F ; Fc1cc(-c2nc(-c3ccccc3)nc(-c3ccccc3)n2)cc(-c2nc(-c3ccccc3)nc(-c3cc(-c4ccccc4)cc(-c4ccccc4)c3)n2)c1F ; Fc1cc(-c2nc(-c3ccccc3)nc(-c3ccccc3)n2)cc(-c2nc(-c3ccccc3)nc(-c3cccc(-c4ccccc4)c3-c3ccccc3)n2)c1F]

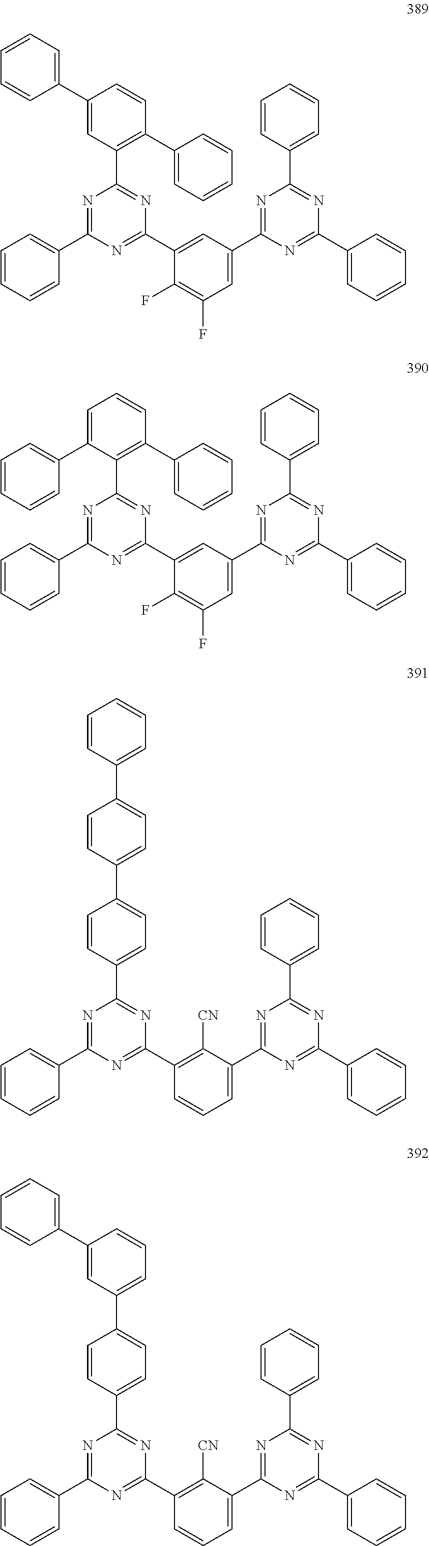 [SMILES: Fc1cc(-c2nc(-c3ccccc3)nc(-c3ccccc3)n2)cc(-c2nc(-c3ccccc3)nc(-c3cc(-c4ccccc4)ccc3-c3ccccc3)n2)c1F ; Fc1cc(-c2nc(-c3ccccc3)nc(-c3ccccc3)n2)cc(-c2nc(-c3ccccc3)nc(-c3c(-c4ccccc4)cccc3-c3ccccc3)n2)c1F ; N#Cc1c(-c2nc(-c3ccccc3)nc(-c3ccccc3)n2)cccc1-c1nc(-c2ccccc2)nc(-c2ccc(-c3ccc(-c4ccccc4)cc3)cc2)n1 ; N#Cc1c(-c2nc(-c3ccccc3)nc(-c3ccccc3)n2)cccc1-c1nc(-c2ccccc2)nc(-c2ccc(-c3cccc(-c4ccccc4)c3)cc2)n1]

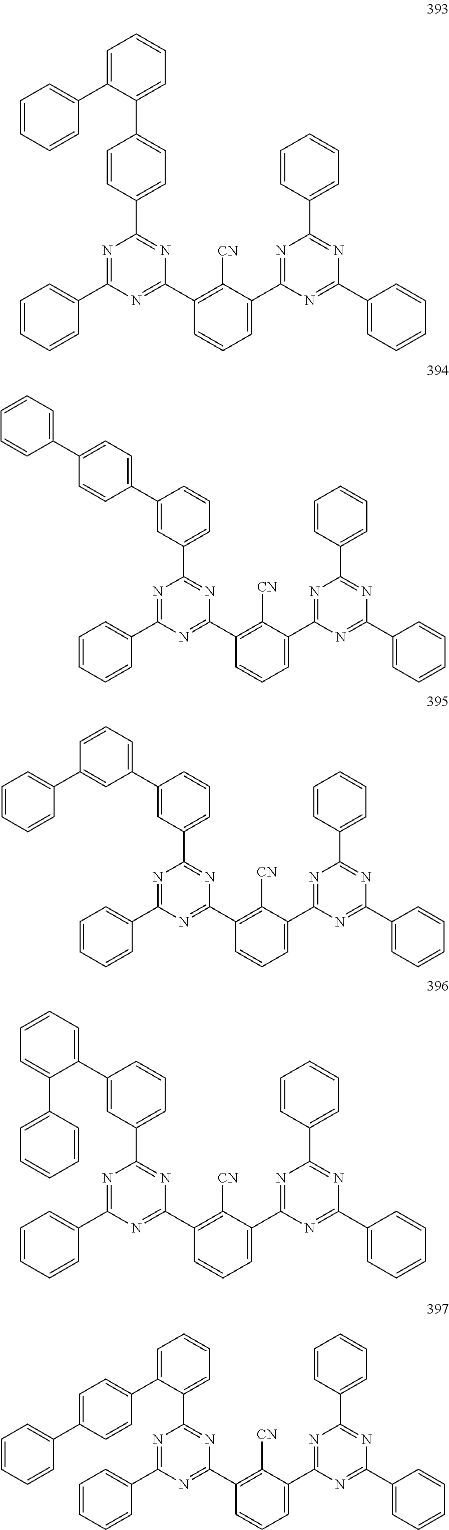 [SMILES: N#Cc1c(-c2nc(-c3ccccc3)nc(-c3ccccc3)n2)cccc1-c1nc(-c2ccccc2)nc(-c2ccc(-c3ccccc3-c3ccccc3)cc2)n1 ; N#Cc1c(-c2nc(-c3ccccc3)nc(-c3ccccc3)n2)cccc1-c1nc(-c2ccccc2)nc(-c2cccc(-c3ccc(-c4ccccc4)cc3)c2)n1 ; N#Cc1c(-c2nc(-c3ccccc3)nc(-c3ccccc3)n2)cccc1-c1nc(-c2ccccc2)nc(-c2cccc(-c3cccc(-c4ccccc4)c3)c2)n1 ; N#Cc1c(-c2nc(-c3ccccc3)nc(-c3ccccc3)n2)cccc1-c1nc(-c2ccccc2)nc(-c2cccc(-c3ccccc3-c3ccccc3)c2)n1 ; N#Cc1c(-c2nc(-c3ccccc3)nc(-c3ccccc3)n2)cccc1-c1nc(-c2ccccc2)nc(-c2ccccc2-c2ccc(-c3ccccc3)cc2)n1]

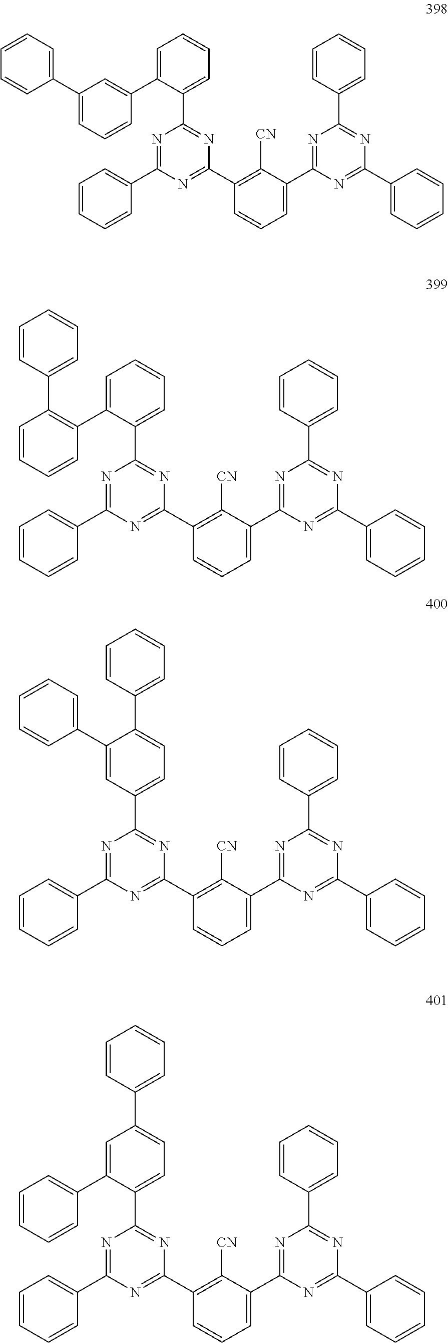 [SMILES: N#Cc1c(-c2nc(-c3ccccc3)nc(-c3ccccc3)n2)cccc1-c1nc(-c2ccccc2)nc(-c2ccccc2-c2cccc(-c3ccccc3)c2)n1 ; N#Cc1c(-c2nc(-c3ccccc3)nc(-c3ccccc3)n2)cccc1-c1nc(-c2ccccc2)nc(-c2ccccc2-c2ccccc2-c2ccccc2)n1 ; N#Cc1c(-c2nc(-c3ccccc3)nc(-c3ccccc3)n2)cccc1-c1nc(-c2ccccc2)nc(-c2ccc(-c3ccccc3)c(-c3ccccc3)c2)n1 ; N#Cc1c(-c2nc(-c3ccccc3)nc(-c3ccccc3)n2)cccc1-c1nc(-c2ccccc2)nc(-c2ccc(-c3ccccc3)cc2-c2ccccc2)n1]

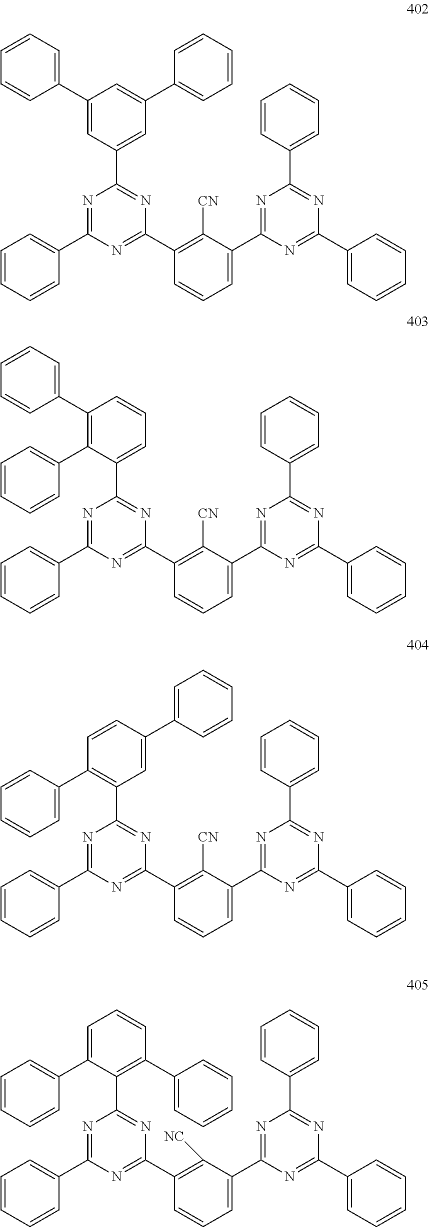

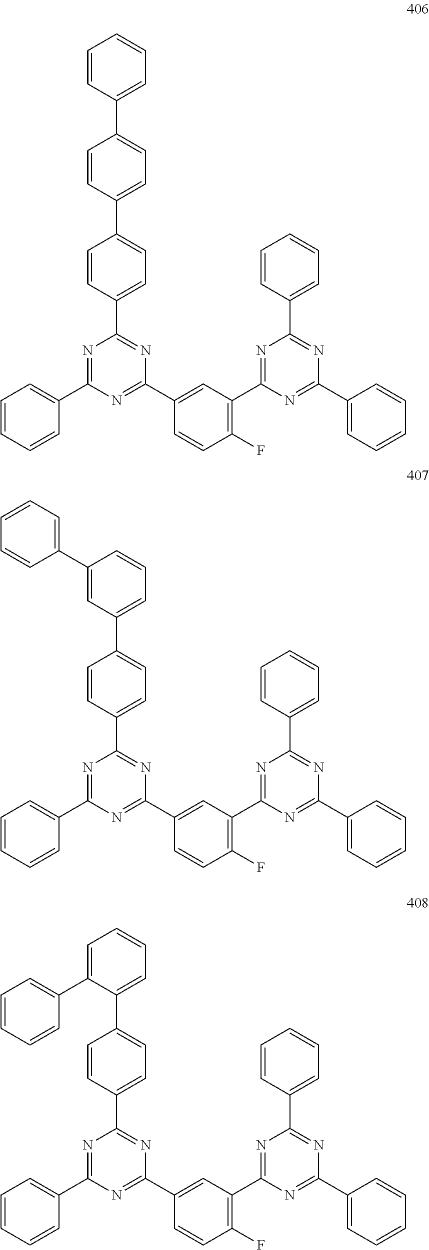 [SMILES: Fc1ccc(-c2nc(-c3ccccc3)nc(-c3ccc(-c4ccc(-c5ccccc5)cc4)cc3)n2)cc1-c1nc(-c2ccccc2)nc(-c2ccccc2)n1 ; Fc1ccc(-c2nc(-c3ccccc3)nc(-c3ccc(-c4cccc(-c5ccccc5)c4)cc3)n2)cc1-c1nc(-c2ccccc2)nc(-c2ccccc2)n1 ; Fc1ccc(-c2nc(-c3ccccc3)nc(-c3ccc(-c4ccccc4-c4ccccc4)cc3)n2)cc1-c1nc(-c2ccccc2)nc(-c2ccccc2)n1]

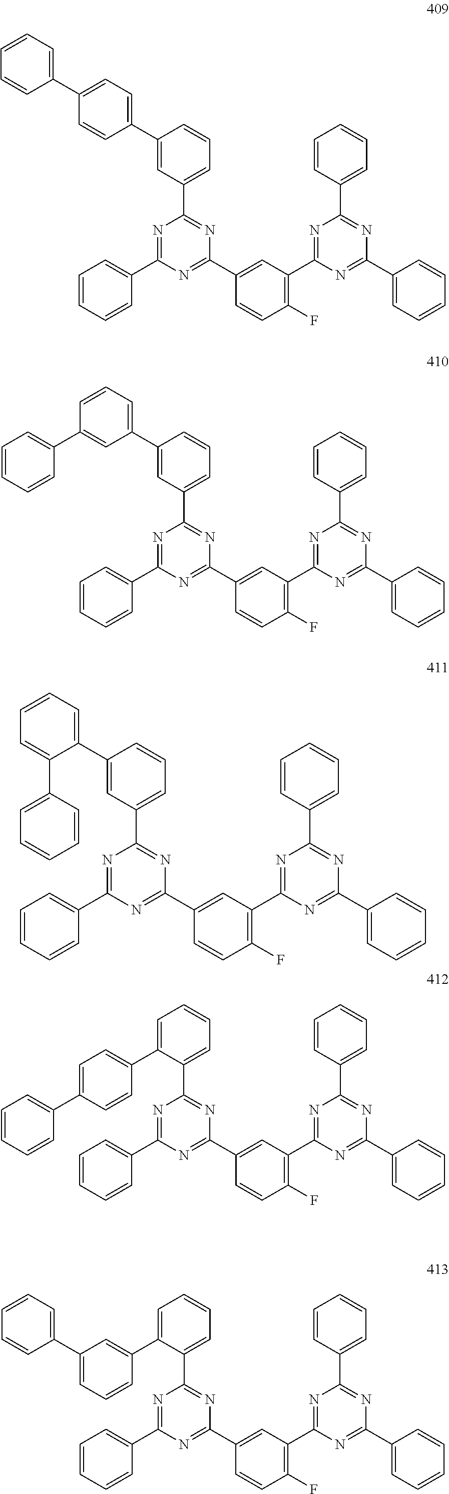 [SMILES: Fc1ccc(-c2nc(-c3ccccc3)nc(-c3cccc(-c4ccc(-c5ccccc5)cc4)c3)n2)cc1-c1nc(-c2ccccc2)nc(-c2ccccc2)n1 ; Fc1ccc(-c2nc(-c3ccccc3)nc(-c3cccc(-c4cccc(-c5ccccc5)c4)c3)n2)cc1-c1nc(-c2ccccc2)nc(-c2ccccc2)n1 ; Fc1ccc(-c2nc(-c3ccccc3)nc(-c3cccc(-c4ccccc4-c4ccccc4)c3)n2)cc1-c1nc(-c2ccccc2)nc(-c2ccccc2)n1 ; Fc1ccc(-c2nc(-c3ccccc3)nc(-c3ccccc3-c3ccc(-c4ccccc4)cc3)n2)cc1-c1nc(-c2ccccc2)nc(-c2ccccc2)n1 ; Fc1ccc(-c2nc(-c3ccccc3)nc(-c3ccccc3-c3cccc(-c4ccccc4)c3)n2)cc1-c1nc(-c2ccccc2)nc(-c2ccccc2)n1]

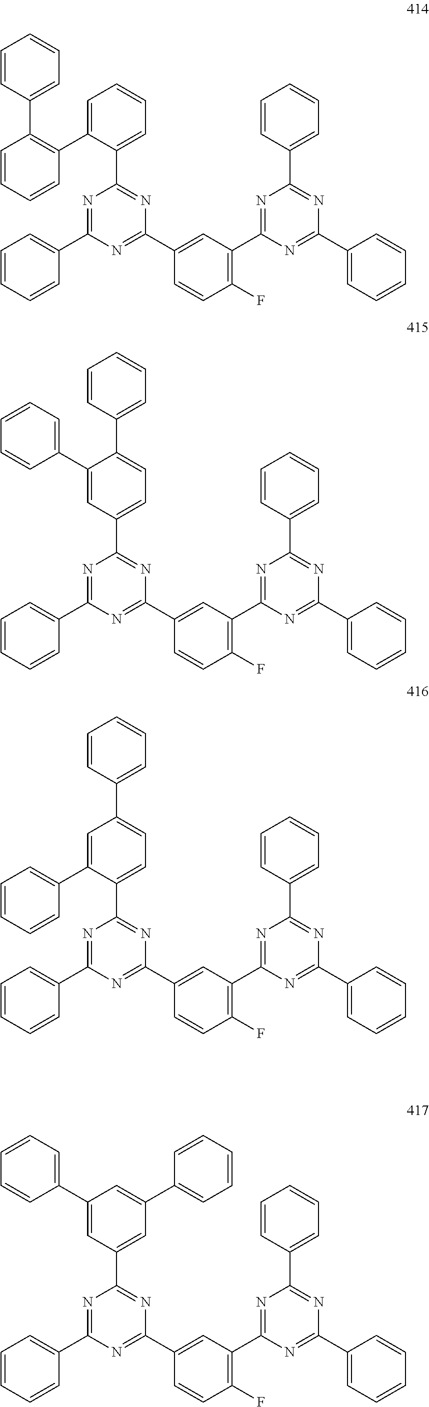 [SMILES: Fc1ccc(-c2nc(-c3ccccc3)nc(-c3ccccc3-c3ccccc3-c3ccccc3)n2)cc1-c1nc(-c2ccccc2)nc(-c2ccccc2)n1 ; Fc1ccc(-c2nc(-c3ccccc3)nc(-c3ccc(-c4ccccc4)c(-c4ccccc4)c3)n2)cc1-c1nc(-c2ccccc2)nc(-c2ccccc2)n1 ; Fc1ccc(-c2nc(-c3ccccc3)nc(-c3ccc(-c4ccccc4)cc3-c3ccccc3)n2)cc1-c1nc(-c2ccccc2)nc(-c2ccccc2)n1 ; Fc1ccc(-c2nc(-c3ccccc3)nc(-c3cc(-c4ccccc4)cc(-c4ccccc4)c3)n2)cc1-c1nc(-c2ccccc2)nc(-c2ccccc2)n1]

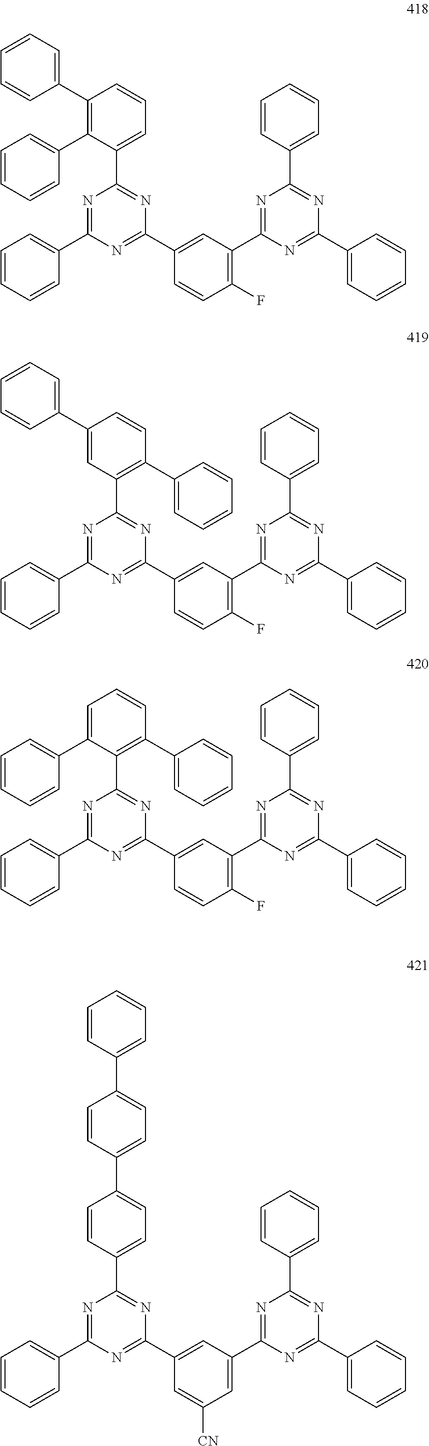 [SMILES: Fc1ccc(-c2nc(-c3ccccc3)nc(-c3cccc(-c4ccccc4)c3-c3ccccc3)n2)cc1-c1nc(-c2ccccc2)nc(-c2ccccc2)n1 ; Fc1ccc(-c2nc(-c3ccccc3)nc(-c3cc(-c4ccccc4)ccc3-c3ccccc3)n2)cc1-c1nc(-c2ccccc2)nc(-c2ccccc2)n1 ; Fc1ccc(-c2nc(-c3ccccc3)nc(-c3c(-c4ccccc4)cccc3-c3ccccc3)n2)cc1-c1nc(-c2ccccc2)nc(-c2ccccc2)n1 ; N#Cc1cc(-c2nc(-c3ccccc3)nc(-c3ccccc3)n2)cc(-c2nc(-c3ccccc3)nc(-c3ccc(-c4ccc(-c5ccccc5)cc4)cc3)n2)c1]

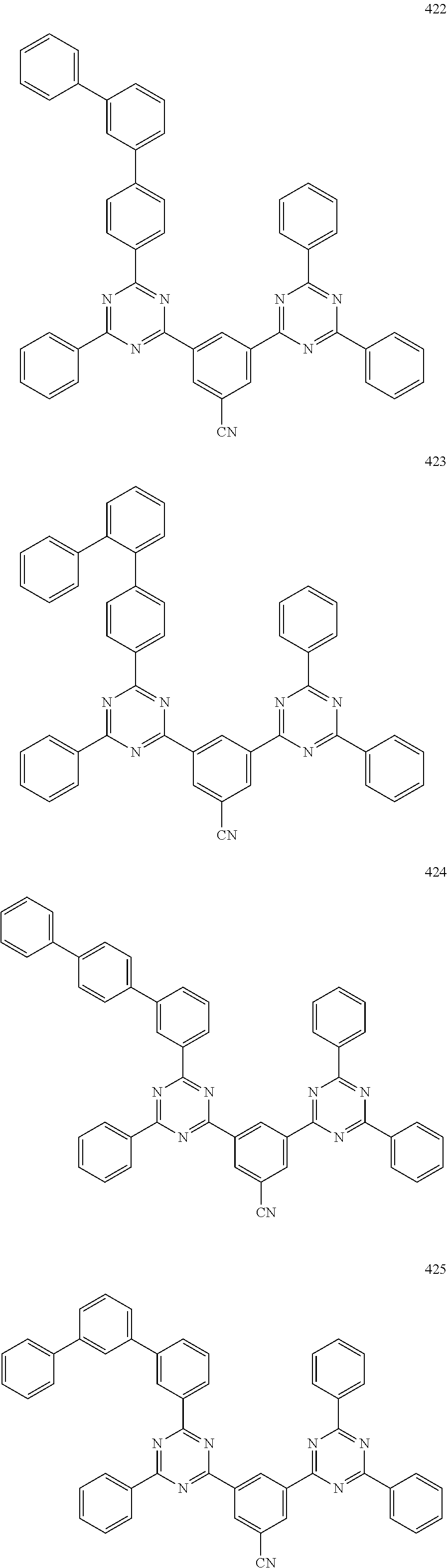 [SMILES: N#Cc1cc(-c2nc(-c3ccccc3)nc(-c3ccccc3)n2)cc(-c2nc(-c3ccccc3)nc(-c3ccc(-c4cccc(-c5ccccc5)c4)cc3)n2)c1 ; N#Cc1cc(-c2nc(-c3ccccc3)nc(-c3ccccc3)n2)cc(-c2nc(-c3ccccc3)nc(-c3ccc(-c4ccccc4-c4ccccc4)cc3)n2)c1 ; N#Cc1cc(-c2nc(-c3ccccc3)nc(-c3ccccc3)n2)cc(-c2nc(-c3ccccc3)nc(-c3cccc(-c4ccc(-c5ccccc5)cc4)c3)n2)c1 ; N#Cc1cc(-c2nc(-c3ccccc3)nc(-c3ccccc3)n2)cc(-c2nc(-c3ccccc3)nc(-c3cccc(-c4cccc(-c5ccccc5)c4)c3)n2)c1]

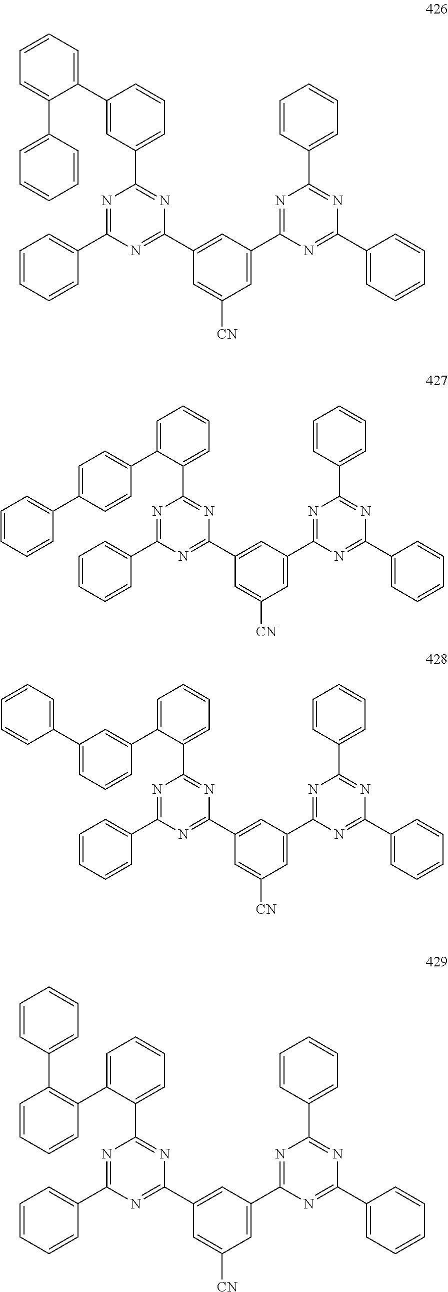 [SMILES: N#Cc1cc(-c2nc(-c3ccccc3)nc(-c3ccccc3)n2)cc(-c2nc(-c3ccccc3)nc(-c3cccc(-c4ccccc4-c4ccccc4)c3)n2)c1 ; N#Cc1cc(-c2nc(-c3ccccc3)nc(-c3ccccc3)n2)cc(-c2nc(-c3ccccc3)nc(-c3ccccc3-c3ccc(-c4ccccc4)cc3)n2)c1 ; N#Cc1cc(-c2nc(-c3ccccc3)nc(-c3ccccc3)n2)cc(-c2nc(-c3ccccc3)nc(-c3ccccc3-c3cccc(-c4ccccc4)c3)n2)c1 ; N#Cc1cc(-c2nc(-c3ccccc3)nc(-c3ccccc3)n2)cc(-c2nc(-c3ccccc3)nc(-c3ccccc3-c3ccccc3-c3ccccc3)n2)c1]

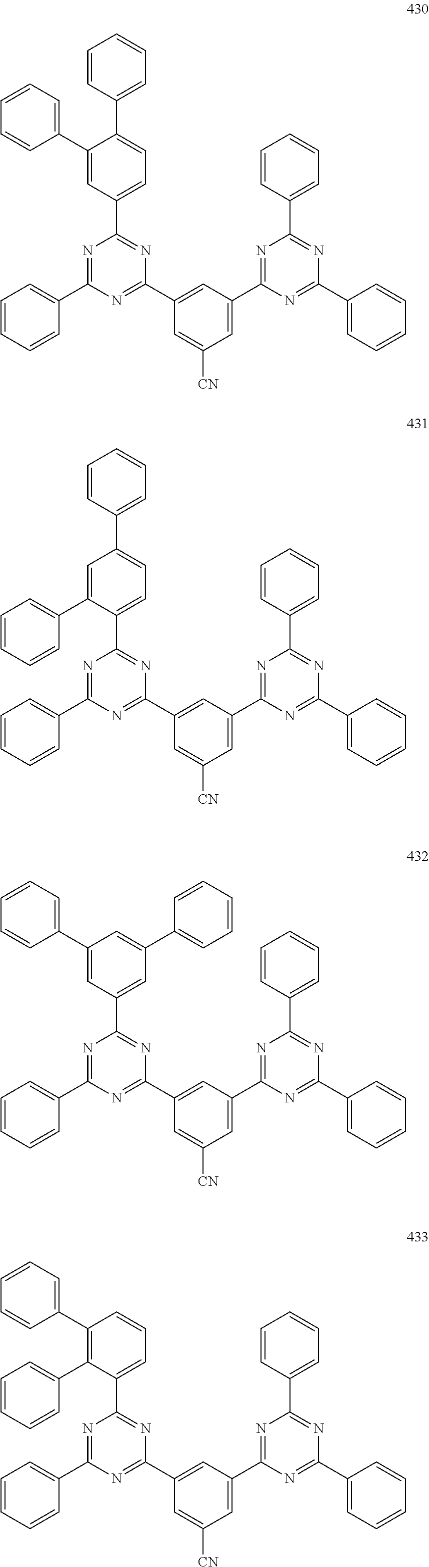 [SMILES: N#Cc1cc(-c2nc(-c3ccccc3)nc(-c3ccccc3)n2)cc(-c2nc(-c3ccccc3)nc(-c3ccc(-c4ccccc4)c(-c4ccccc4)c3)n2)c1 ; N#Cc1cc(-c2nc(-c3ccccc3)nc(-c3ccccc3)n2)cc(-c2nc(-c3ccccc3)nc(-c3ccc(-c4ccccc4)cc3-c3ccccc3)n2)c1 ; N#Cc1cc(-c2nc(-c3ccccc3)nc(-c3ccccc3)n2)cc(-c2nc(-c3ccccc3)nc(-c3cc(-c4ccccc4)cc(-c4ccccc4)c3)n2)c1 ; N#Cc1cc(-c2nc(-c3ccccc3)nc(-c3ccccc3)n2)cc(-c2nc(-c3ccccc3)nc(-c3cccc(-c4ccccc4)c3-c3ccccc3)n2)c1]

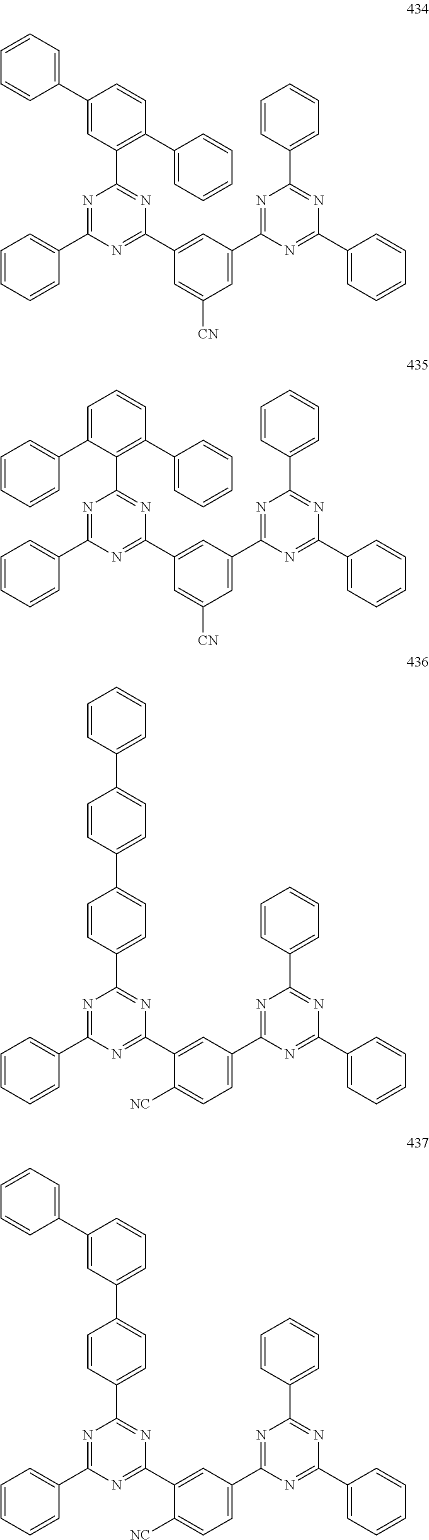 [SMILES: N#Cc1cc(-c2nc(-c3ccccc3)nc(-c3ccccc3)n2)cc(-c2nc(-c3ccccc3)nc(-c3cc(-c4ccccc4)ccc3-c3ccccc3)n2)c1 ; N#Cc1cc(-c2nc(-c3ccccc3)nc(-c3ccccc3)n2)cc(-c2nc(-c3ccccc3)nc(-c3c(-c4ccccc4)cccc3-c3ccccc3)n2)c1 ; N#Cc1ccc(-c2nc(-c3ccccc3)nc(-c3ccccc3)n2)cc1-c1nc(-c2ccccc2)nc(-c2ccc(-c3ccc(-c4ccccc4)cc3)cc2)n1 ; N#Cc1ccc(-c2nc(-c3ccccc3)nc(-c3ccccc3)n2)cc1-c1nc(-c2ccccc2)nc(-c2ccc(-c3cccc(-c4ccccc4)c3)cc2)n1]

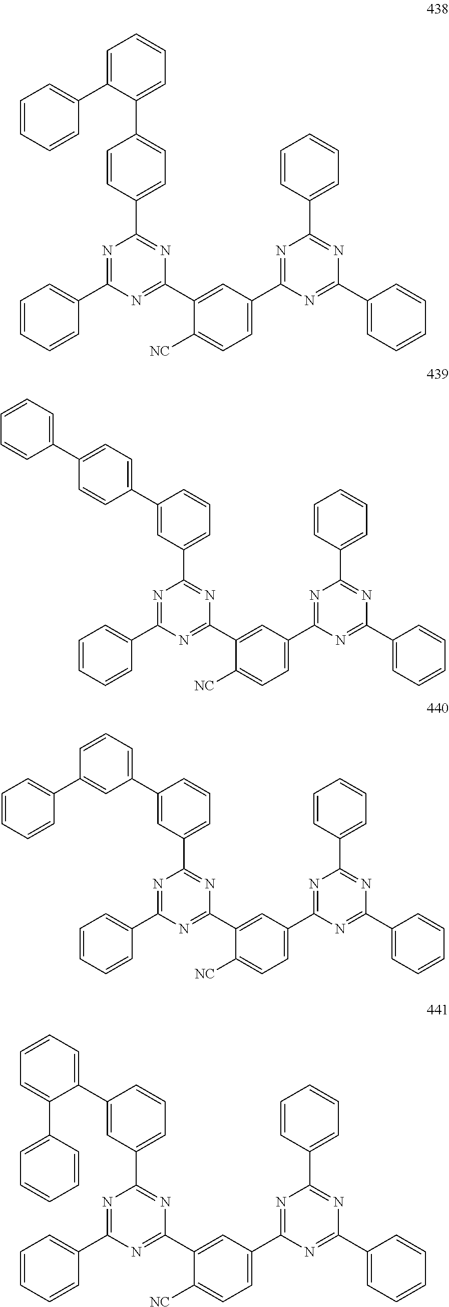 [SMILES: N#Cc1ccc(-c2nc(-c3ccccc3)nc(-c3ccccc3)n2)cc1-c1nc(-c2ccccc2)nc(-c2ccc(-c3ccccc3-c3ccccc3)cc2)n1 ; N#Cc1ccc(-c2nc(-c3ccccc3)nc(-c3ccccc3)n2)cc1-c1nc(-c2ccccc2)nc(-c2cccc(-c3ccc(-c4ccccc4)cc3)c2)n1 ; N#Cc1ccc(-c2nc(-c3ccccc3)nc(-c3ccccc3)n2)cc1-c1nc(-c2ccccc2)nc(-c2cccc(-c3cccc(-c4ccccc4)c3)c2)n1 ; N#Cc1ccc(-c2nc(-c3ccccc3)nc(-c3ccccc3)n2)cc1-c1nc(-c2ccccc2)nc(-c2cccc(-c3ccccc3-c3ccccc3)c2)n1]

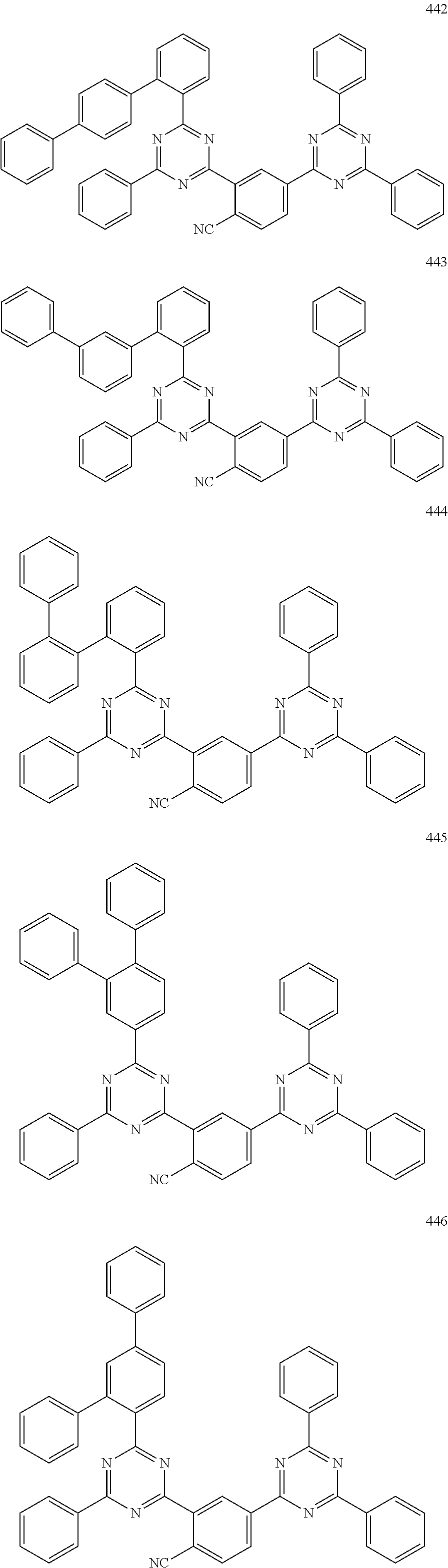 [SMILES: N#Cc1ccc(-c2nc(-c3ccccc3)nc(-c3ccccc3)n2)cc1-c1nc(-c2ccccc2)nc(-c2ccccc2-c2ccc(-c3ccccc3)cc2)n1 ; N#Cc1ccc(-c2nc(-c3ccccc3)nc(-c3ccccc3)n2)cc1-c1nc(-c2ccccc2)nc(-c2ccccc2-c2cccc(-c3ccccc3)c2)n1 ; N#Cc1ccc(-c2nc(-c3ccccc3)nc(-c3ccccc3)n2)cc1-c1nc(-c2ccccc2)nc(-c2ccccc2-c2ccccc2-c2ccccc2)n1 ; N#Cc1ccc(-c2nc(-c3ccccc3)nc(-c3ccccc3)n2)cc1-c1nc(-c2ccccc2)nc(-c2ccc(-c3ccccc3)c(-c3ccccc3)c2)n1 ; N#Cc1ccc(-c2nc(-c3ccccc3)nc(-c3ccccc3)n2)cc1-c1nc(-c2ccccc2)nc(-c2ccc(-c3ccccc3)cc2-c2ccccc2)n1]

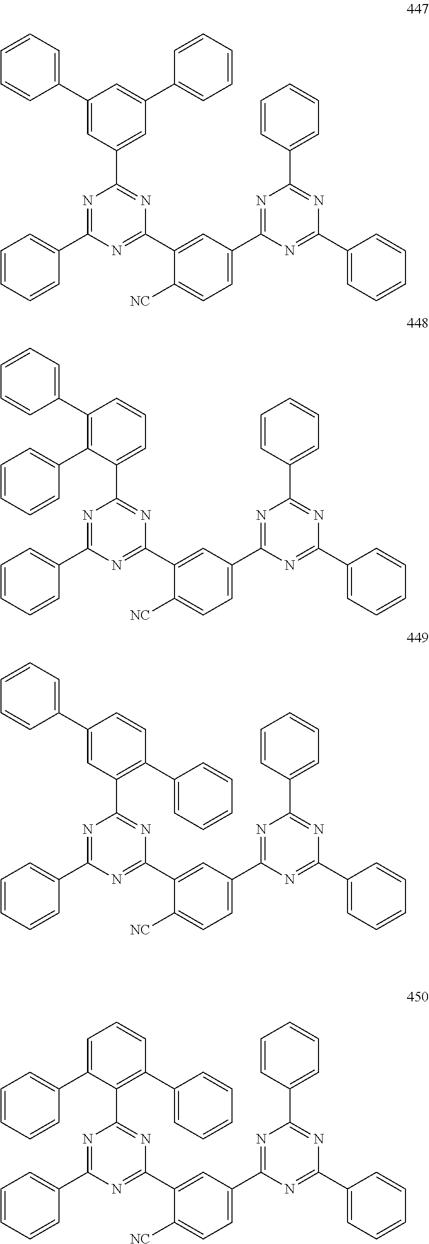 [SMILES: N#Cc1ccc(-c2nc(-c3ccccc3)nc(-c3ccccc3)n2)cc1-c1nc(-c2ccccc2)nc(-c2cc(-c3ccccc3)cc(-c3ccccc3)c2)n1 ; N#Cc1ccc(-c2nc(-c3ccccc3)nc(-c3ccccc3)n2)cc1-c1nc(-c2ccccc2)nc(-c2cccc(-c3ccccc3)c2-c2ccccc2)n1 ; N#Cc1ccc(-c2nc(-c3ccccc3)nc(-c3ccccc3)n2)cc1-c1nc(-c2ccccc2)nc(-c2cc(-c3ccccc3)ccc2-c2ccccc2)n1 ; N#Cc1ccc(-c2nc(-c3ccccc3)nc(-c3ccccc3)n2)cc1-c1nc(-c2ccccc2)nc(-c2c(-c3ccccc3)cccc2-c2ccccc2)n1]

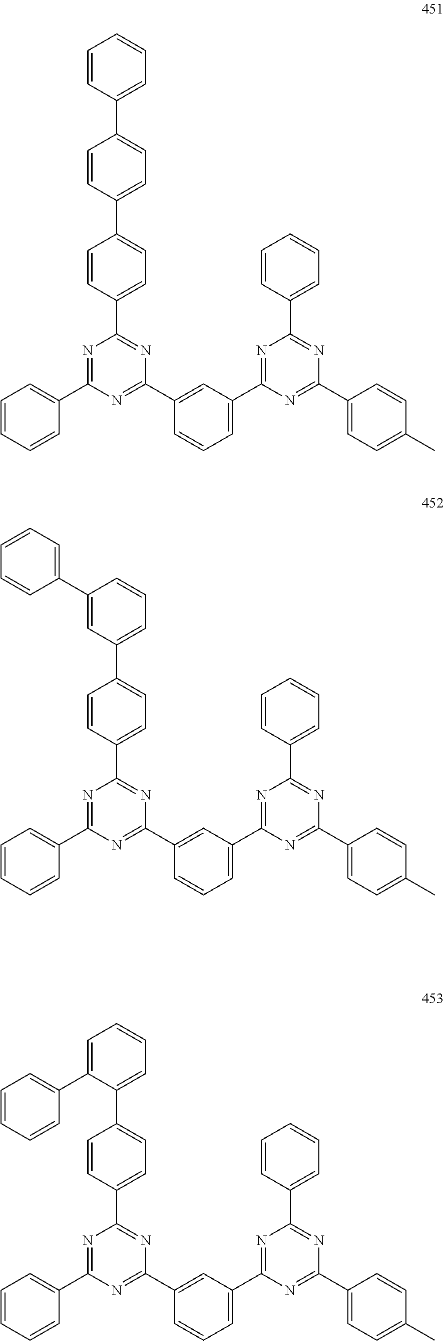 [SMILES: Cc1ccc(-c2nc(-c3ccccc3)nc(-c3cccc(-c4nc(-c5ccccc5)nc(-c5ccc(-c6ccc(-c7ccccc7)cc6)cc5)n4)c3)n2)cc1 ; Cc1ccc(-c2nc(-c3ccccc3)nc(-c3cccc(-c4nc(-c5ccccc5)nc(-c5ccc(-c6cccc(-c7ccccc7)c6)cc5)n4)c3)n2)cc1 ; Cc1ccc(-c2nc(-c3ccccc3)nc(-c3cccc(-c4nc(-c5ccccc5)nc(-c5ccc(-c6ccccc6-c6ccccc6)cc5)n4)c3)n2)cc1]

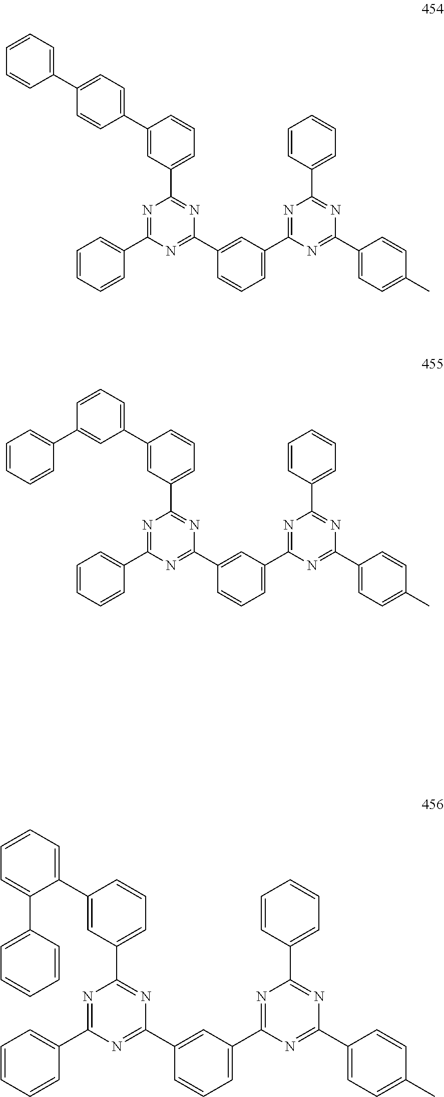 [SMILES: Cc1ccc(-c2nc(-c3ccccc3)nc(-c3cccc(-c4nc(-c5ccccc5)nc(-c5cccc(-c6ccc(-c7ccccc7)cc6)c5)n4)c3)n2)cc1 ; Cc1ccc(-c2nc(-c3ccccc3)nc(-c3cccc(-c4nc(-c5ccccc5)nc(-c5cccc(-c6cccc(-c7ccccc7)c6)c5)n4)c3)n2)cc1 ; Cc1ccc(-c2nc(-c3ccccc3)nc(-c3cccc(-c4nc(-c5ccccc5)nc(-c5cccc(-c6ccccc6-c6ccccc6)c5)n4)c3)n2)cc1]

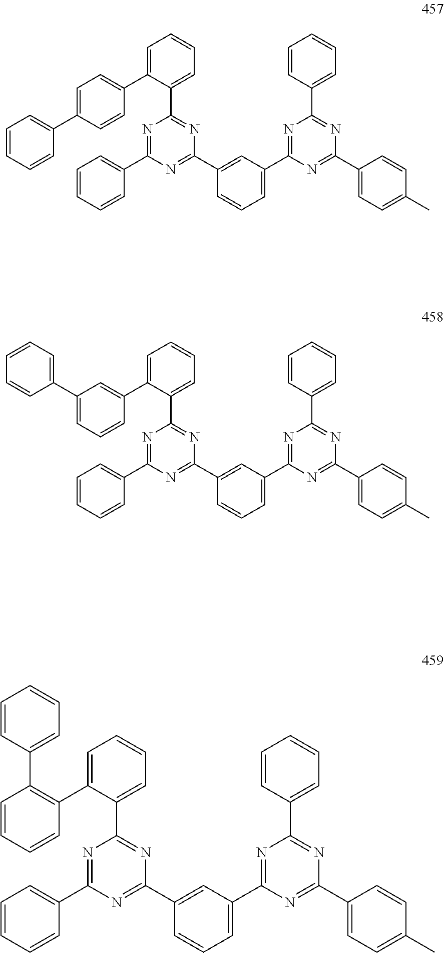 [SMILES: Cc1ccc(-c2nc(-c3ccccc3)nc(-c3cccc(-c4nc(-c5ccccc5)nc(-c5ccccc5-c5ccc(-c6ccccc6)cc5)n4)c3)n2)cc1 ; Cc1ccc(-c2nc(-c3ccccc3)nc(-c3cccc(-c4nc(-c5ccccc5)nc(-c5ccccc5-c5cccc(-c6ccccc6)c5)n4)c3)n2)cc1 ; Cc1ccc(-c2nc(-c3ccccc3)nc(-c3cccc(-c4nc(-c5ccccc5)nc(-c5ccccc5-c5ccccc5-c5ccccc5)n4)c3)n2)cc1]

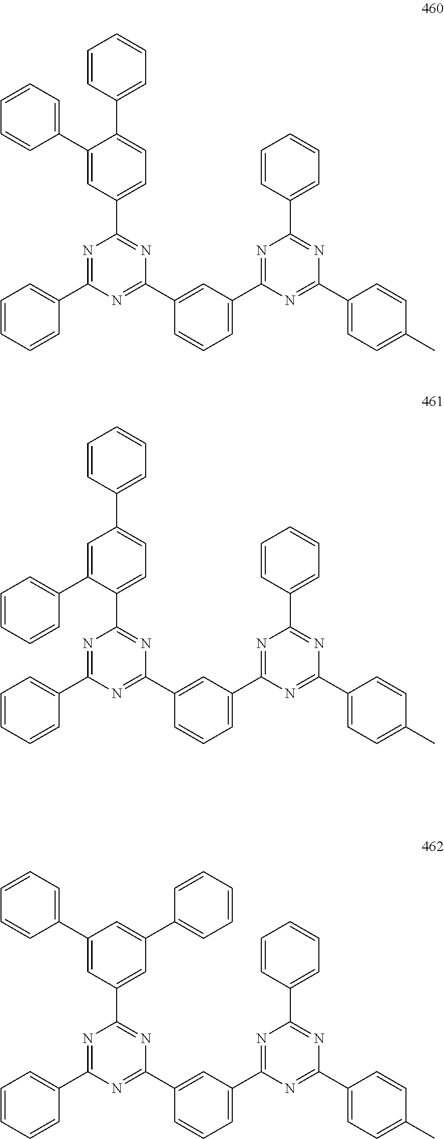 [SMILES: Cc1ccc(-c2nc(-c3ccccc3)nc(-c3cccc(-c4nc(-c5ccccc5)nc(-c5ccc(-c6ccccc6)c(-c6ccccc6)c5)n4)c3)n2)cc1 ; Cc1ccc(-c2nc(-c3ccccc3)nc(-c3cccc(-c4nc(-c5ccccc5)nc(-c5ccc(-c6ccccc6)cc5-c5ccccc5)n4)c3)n2)cc1 ; Cc1ccc(-c2nc(-c3ccccc3)nc(-c3cccc(-c4nc(-c5ccccc5)nc(-c5cc(-c6ccccc6)cc(-c6ccccc6)c5)n4)c3)n2)cc1]

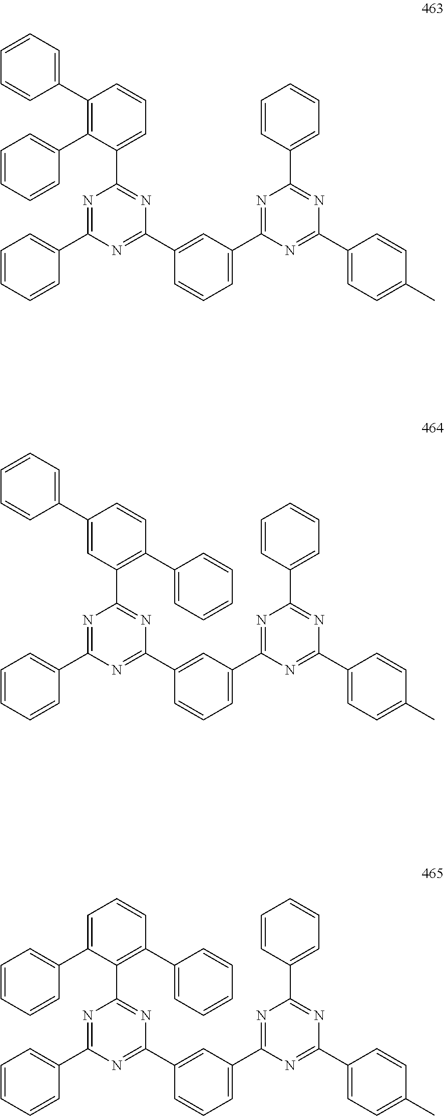 [SMILES: Cc1ccc(-c2nc(-c3ccccc3)nc(-c3cccc(-c4nc(-c5ccccc5)nc(-c5cccc(-c6ccccc6)c5-c5ccccc5)n4)c3)n2)cc1 ; Cc1ccc(-c2nc(-c3ccccc3)nc(-c3cccc(-c4nc(-c5ccccc5)nc(-c5cc(-c6ccccc6)ccc5-c5ccccc5)n4)c3)n2)cc1 ; Cc1ccc(-c2nc(-c3ccccc3)nc(-c3cccc(-c4nc(-c5ccccc5)nc(-c5c(-c6ccccc6)cccc5-c5ccccc5)n4)c3)n2)cc1]

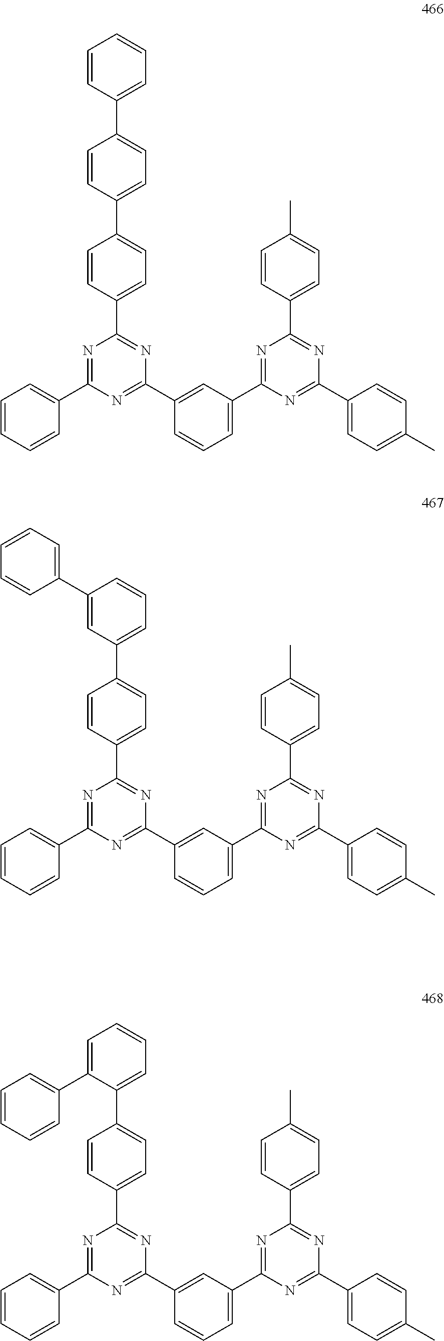 [SMILES: Cc1ccc(-c2nc(-c3ccc(C)cc3)nc(-c3cccc(-c4nc(-c5ccccc5)nc(-c5ccc(-c6ccc(-c7ccccc7)cc6)cc5)n4)c3)n2)cc1 ; Cc1ccc(-c2nc(-c3ccc(C)cc3)nc(-c3cccc(-c4nc(-c5ccccc5)nc(-c5ccc(-c6cccc(-c7ccccc7)c6)cc5)n4)c3)n2)cc1 ; Cc1ccc(-c2nc(-c3ccc(C)cc3)nc(-c3cccc(-c4nc(-c5ccccc5)nc(-c5ccc(-c6ccccc6-c6ccccc6)cc5)n4)c3)n2)cc1]

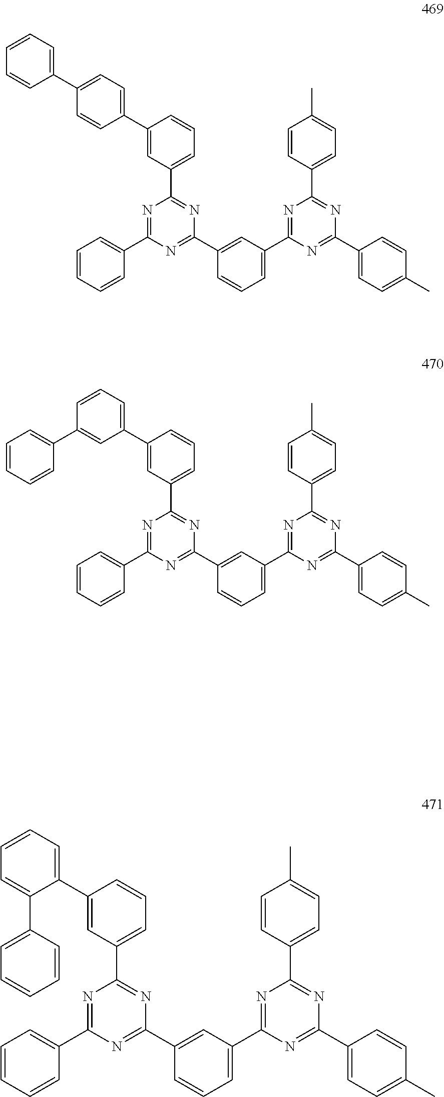 [SMILES: Cc1ccc(-c2nc(-c3ccc(C)cc3)nc(-c3cccc(-c4nc(-c5ccccc5)nc(-c5cccc(-c6ccc(-c7ccccc7)cc6)c5)n4)c3)n2)cc1 ; Cc1ccc(-c2nc(-c3ccc(C)cc3)nc(-c3cccc(-c4nc(-c5ccccc5)nc(-c5cccc(-c6cccc(-c7ccccc7)c6)c5)n4)c3)n2)cc1 ; Cc1ccc(-c2nc(-c3ccc(C)cc3)nc(-c3cccc(-c4nc(-c5ccccc5)nc(-c5cccc(-c6ccccc6-c6ccccc6)c5)n4)c3)n2)cc1]

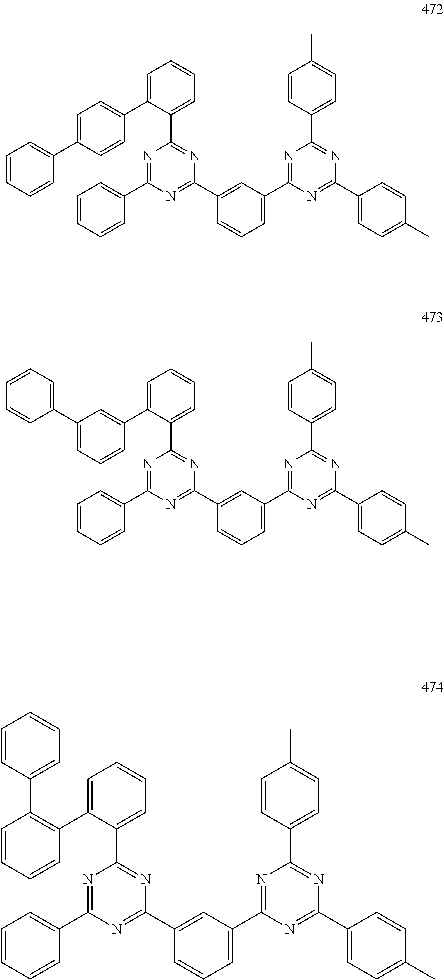 [SMILES: Cc1ccc(-c2nc(-c3ccc(C)cc3)nc(-c3cccc(-c4nc(-c5ccccc5)nc(-c5ccccc5-c5ccc(-c6ccccc6)cc5)n4)c3)n2)cc1 ; Cc1ccc(-c2nc(-c3ccc(C)cc3)nc(-c3cccc(-c4nc(-c5ccccc5)nc(-c5ccccc5-c5cccc(-c6ccccc6)c5)n4)c3)n2)cc1 ; Cc1ccc(-c2nc(-c3ccc(C)cc3)nc(-c3cccc(-c4nc(-c5ccccc5)nc(-c5ccccc5-c5ccccc5-c5ccccc5)n4)c3)n2)cc1]

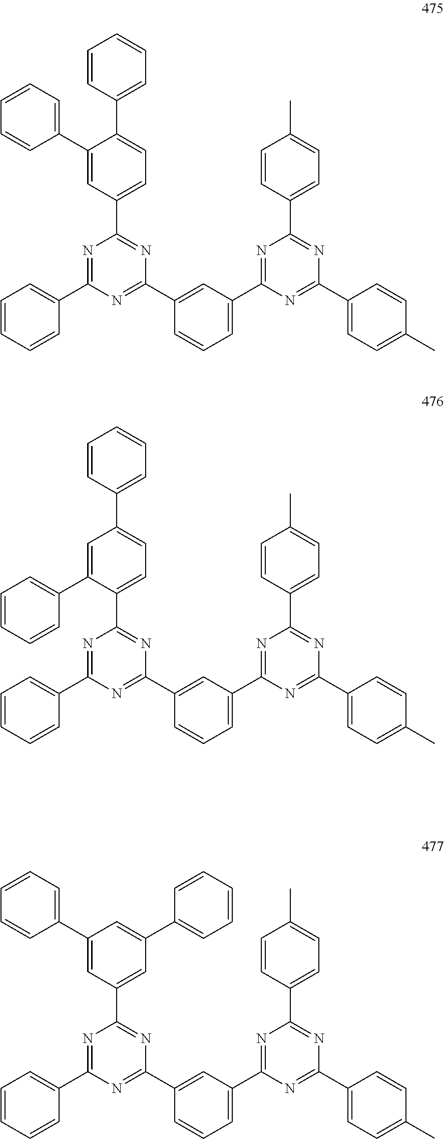 [SMILES: Cc1ccc(-c2nc(-c3ccc(C)cc3)nc(-c3cccc(-c4nc(-c5ccccc5)nc(-c5ccc(-c6ccccc6)c(-c6ccccc6)c5)n4)c3)n2)cc1 ; Cc1ccc(-c2nc(-c3ccc(C)cc3)nc(-c3cccc(-c4nc(-c5ccccc5)nc(-c5ccc(-c6ccccc6)cc5-c5ccccc5)n4)c3)n2)cc1 ; Cc1ccc(-c2nc(-c3ccc(C)cc3)nc(-c3cccc(-c4nc(-c5ccccc5)nc(-c5cc(-c6ccccc6)cc(-c6ccccc6)c5)n4)c3)n2)cc1]

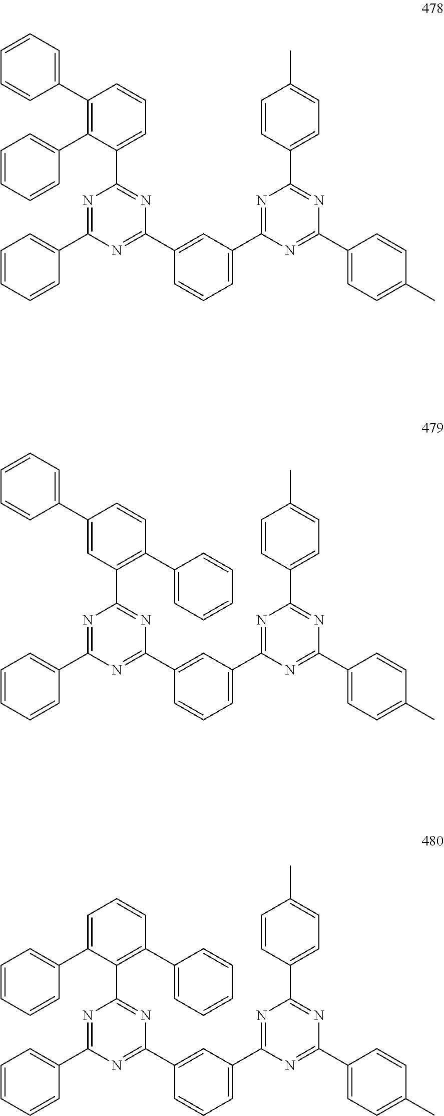 [SMILES: Cc1ccc(-c2nc(-c3ccc(C)cc3)nc(-c3cccc(-c4nc(-c5ccccc5)nc(-c5cccc(-c6ccccc6)c5-c5ccccc5)n4)c3)n2)cc1 ; Cc1ccc(-c2nc(-c3ccc(C)cc3)nc(-c3cccc(-c4nc(-c5ccccc5)nc(-c5cc(-c6ccccc6)ccc5-c5ccccc5)n4)c3)n2)cc1 ; Cc1ccc(-c2nc(-c3ccc(C)cc3)nc(-c3cccc(-c4nc(-c5ccccc5)nc(-c5c(-c6ccccc6)cccc5-c5ccccc5)n4)c3)n2)cc1]

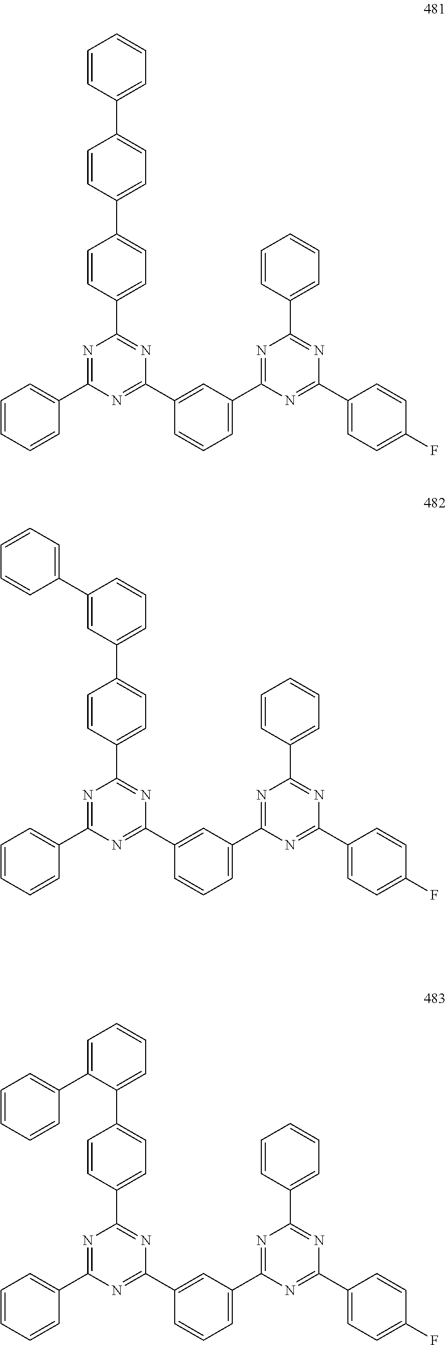 [SMILES: Fc1ccc(-c2nc(-c3ccccc3)nc(-c3cccc(-c4nc(-c5ccccc5)nc(-c5ccc(-c6ccc(-c7ccccc7)cc6)cc5)n4)c3)n2)cc1 ; Fc1ccc(-c2nc(-c3ccccc3)nc(-c3cccc(-c4nc(-c5ccccc5)nc(-c5ccc(-c6cccc(-c7ccccc7)c6)cc5)n4)c3)n2)cc1 ; Fc1ccc(-c2nc(-c3ccccc3)nc(-c3cccc(-c4nc(-c5ccccc5)nc(-c5ccc(-c6ccccc6-c6ccccc6)cc5)n4)c3)n2)cc1]

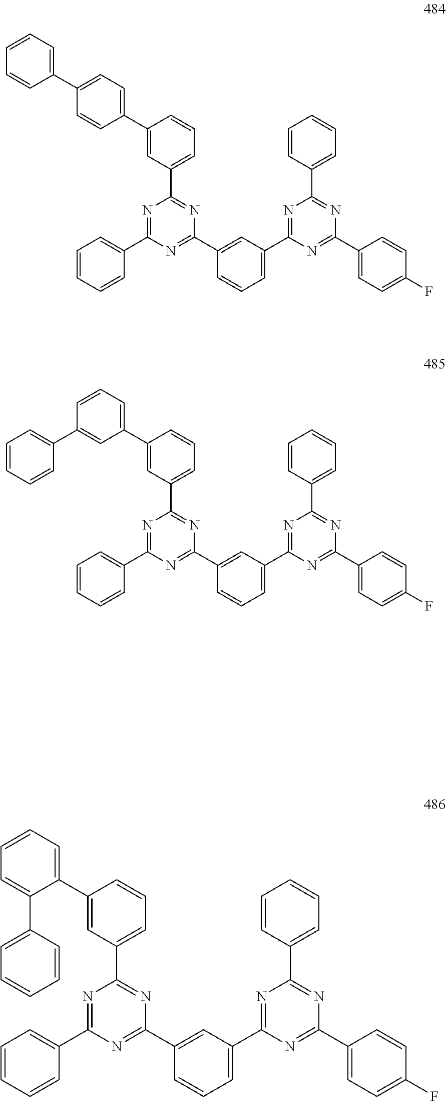 [SMILES: Fc1ccc(-c2nc(-c3ccccc3)nc(-c3cccc(-c4nc(-c5ccccc5)nc(-c5cccc(-c6ccc(-c7ccccc7)cc6)c5)n4)c3)n2)cc1 ; Fc1ccc(-c2nc(-c3ccccc3)nc(-c3cccc(-c4nc(-c5ccccc5)nc(-c5cccc(-c6cccc(-c7ccccc7)c6)c5)n4)c3)n2)cc1 ; Fc1ccc(-c2nc(-c3ccccc3)nc(-c3cccc(-c4nc(-c5ccccc5)nc(-c5cccc(-c6ccccc6-c6ccccc6)c5)n4)c3)n2)cc1]

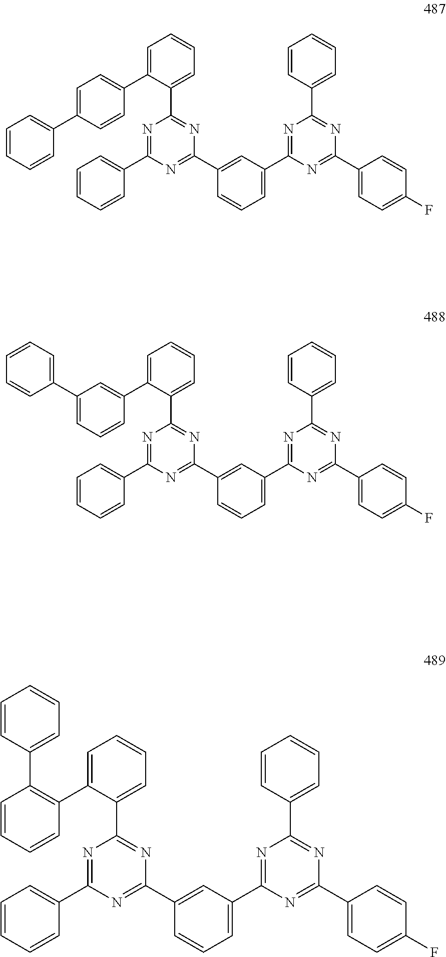 [SMILES: Fc1ccc(-c2nc(-c3ccccc3)nc(-c3cccc(-c4nc(-c5ccccc5)nc(-c5ccccc5-c5ccc(-c6ccccc6)cc5)n4)c3)n2)cc1 ; Fc1ccc(-c2nc(-c3ccccc3)nc(-c3cccc(-c4nc(-c5ccccc5)nc(-c5ccccc5-c5cccc(-c6ccccc6)c5)n4)c3)n2)cc1 ; Fc1ccc(-c2nc(-c3ccccc3)nc(-c3cccc(-c4nc(-c5ccccc5)nc(-c5ccccc5-c5ccccc5-c5ccccc5)n4)c3)n2)cc1]

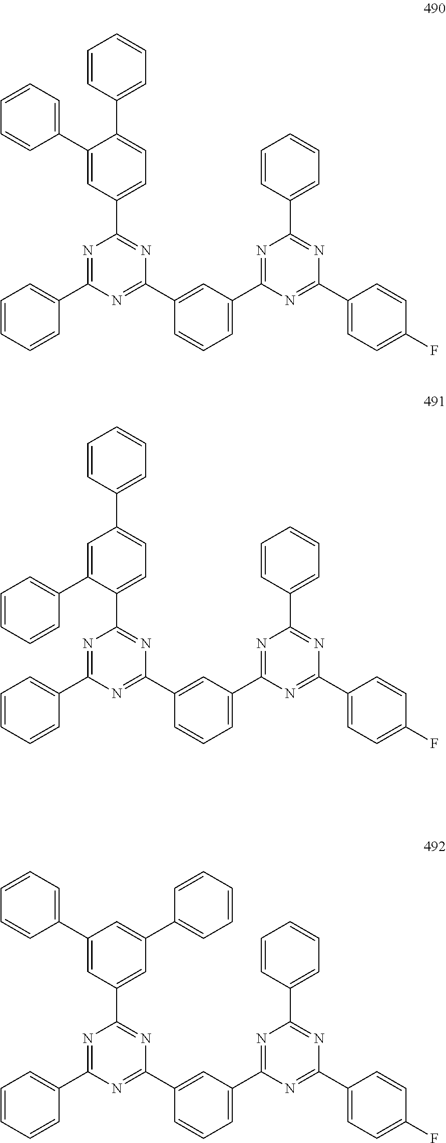 [SMILES: Fc1ccc(-c2nc(-c3ccccc3)nc(-c3cccc(-c4nc(-c5ccccc5)nc(-c5ccc(-c6ccccc6)c(-c6ccccc6)c5)n4)c3)n2)cc1 ; Fc1ccc(-c2nc(-c3ccccc3)nc(-c3cccc(-c4nc(-c5ccccc5)nc(-c5ccc(-c6ccccc6)cc5-c5ccccc5)n4)c3)n2)cc1 ; Fc1ccc(-c2nc(-c3ccccc3)nc(-c3cccc(-c4nc(-c5ccccc5)nc(-c5cc(-c6ccccc6)cc(-c6ccccc6)c5)n4)c3)n2)cc1]

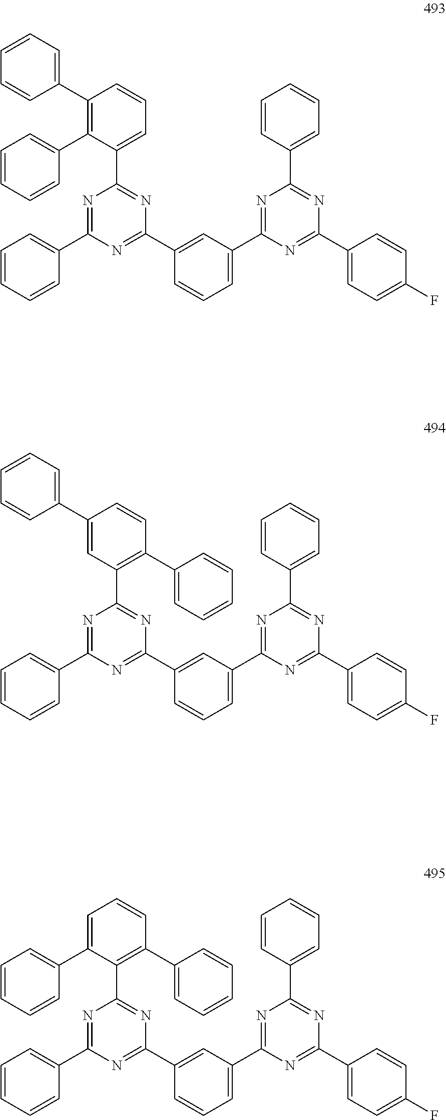 [SMILES: Fc1ccc(-c2nc(-c3ccccc3)nc(-c3cccc(-c4nc(-c5ccccc5)nc(-c5cccc(-c6ccccc6)c5-c5ccccc5)n4)c3)n2)cc1 ; Fc1ccc(-c2nc(-c3ccccc3)nc(-c3cccc(-c4nc(-c5ccccc5)nc(-c5cc(-c6ccccc6)ccc5-c5ccccc5)n4)c3)n2)cc1 ; Fc1ccc(-c2nc(-c3ccccc3)nc(-c3cccc(-c4nc(-c5ccccc5)nc(-c5c(-c6ccccc6)cccc5-c5ccccc5)n4)c3)n2)cc1]

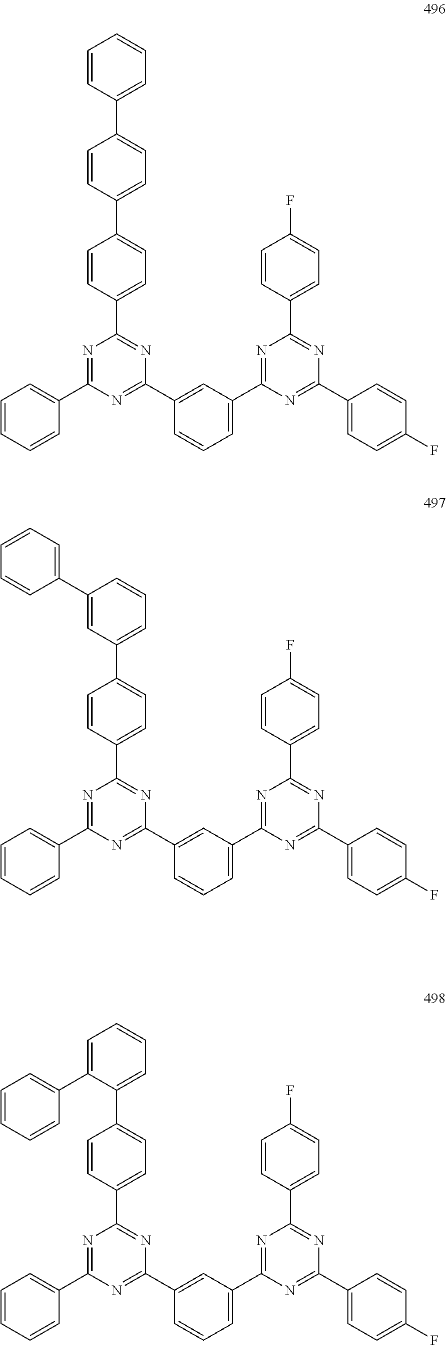 [SMILES: Fc1ccc(-c2nc(-c3ccc(F)cc3)nc(-c3cccc(-c4nc(-c5ccccc5)nc(-c5ccc(-c6ccc(-c7ccccc7)cc6)cc5)n4)c3)n2)cc1 ; Fc1ccc(-c2nc(-c3ccc(F)cc3)nc(-c3cccc(-c4nc(-c5ccccc5)nc(-c5ccc(-c6cccc(-c7ccccc7)c6)cc5)n4)c3)n2)cc1 ; Fc1ccc(-c2nc(-c3ccc(F)cc3)nc(-c3cccc(-c4nc(-c5ccccc5)nc(-c5ccc(-c6ccccc6-c6ccccc6)cc5)n4)c3)n2)cc1]

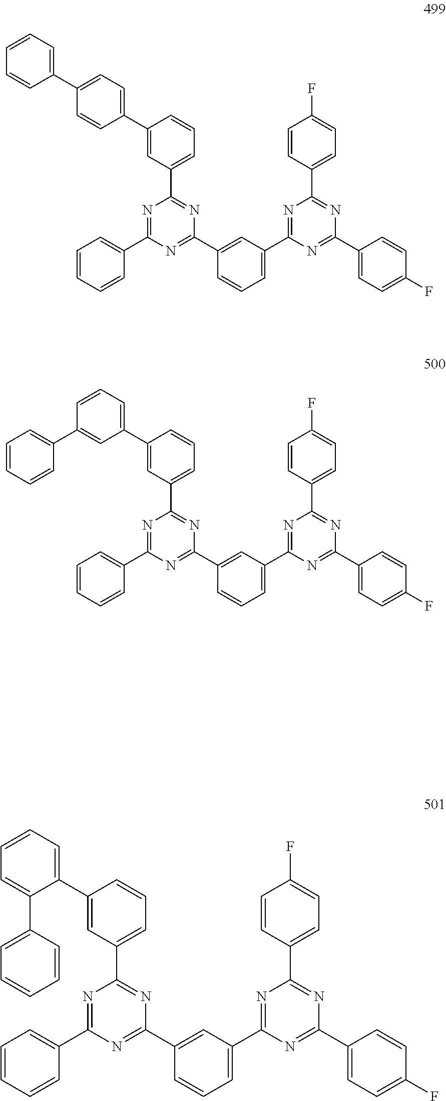 [SMILES: Fc1ccc(-c2nc(-c3ccc(F)cc3)nc(-c3cccc(-c4nc(-c5ccccc5)nc(-c5cccc(-c6ccc(-c7ccccc7)cc6)c5)n4)c3)n2)cc1 ; Fc1ccc(-c2nc(-c3ccc(F)cc3)nc(-c3cccc(-c4nc(-c5ccccc5)nc(-c5cccc(-c6cccc(-c7ccccc7)c6)c5)n4)c3)n2)cc1 ; Fc1ccc(-c2nc(-c3ccc(F)cc3)nc(-c3cccc(-c4nc(-c5ccccc5)nc(-c5cccc(-c6ccccc6-c6ccccc6)c5)n4)c3)n2)cc1]

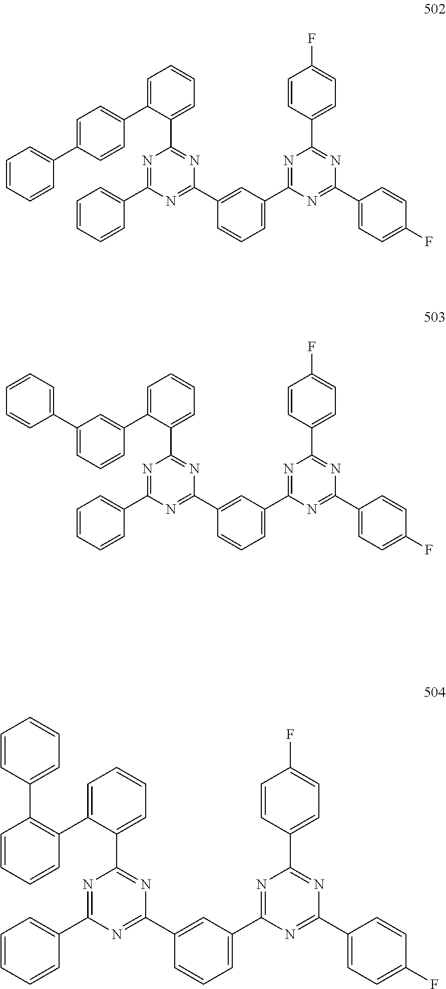 [SMILES: Fc1ccc(-c2nc(-c3ccc(F)cc3)nc(-c3cccc(-c4nc(-c5ccccc5)nc(-c5ccccc5-c5ccc(-c6ccccc6)cc5)n4)c3)n2)cc1 ; Fc1ccc(-c2nc(-c3ccc(F)cc3)nc(-c3cccc(-c4nc(-c5ccccc5)nc(-c5ccccc5-c5cccc(-c6ccccc6)c5)n4)c3)n2)cc1 ; Fc1ccc(-c2nc(-c3ccc(F)cc3)nc(-c3cccc(-c4nc(-c5ccccc5)nc(-c5ccccc5-c5ccccc5-c5ccccc5)n4)c3)n2)cc1]

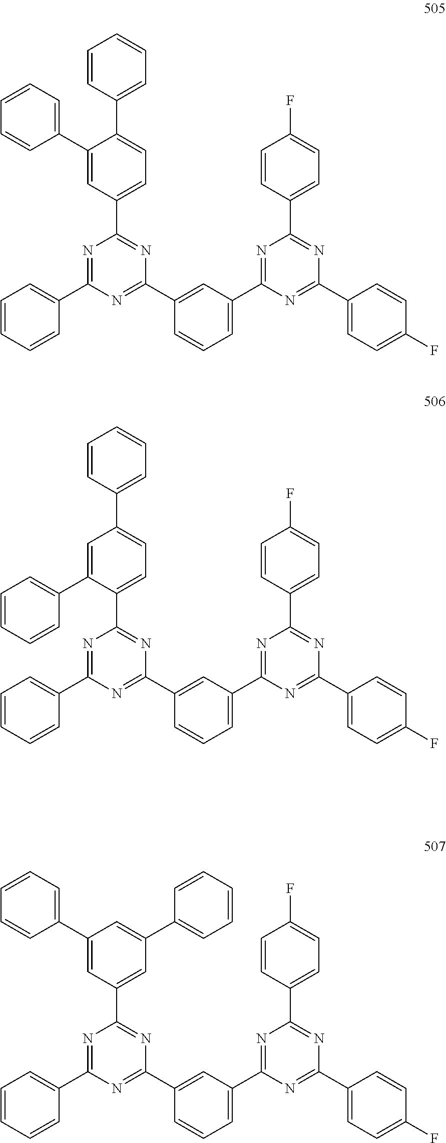 [SMILES: Fc1ccc(-c2nc(-c3ccc(F)cc3)nc(-c3cccc(-c4nc(-c5ccccc5)nc(-c5ccc(-c6ccccc6)c(-c6ccccc6)c5)n4)c3)n2)cc1 ; Fc1ccc(-c2nc(-c3ccc(F)cc3)nc(-c3cccc(-c4nc(-c5ccccc5)nc(-c5ccc(-c6ccccc6)cc5-c5ccccc5)n4)c3)n2)cc1 ; Fc1ccc(-c2nc(-c3ccc(F)cc3)nc(-c3cccc(-c4nc(-c5ccccc5)nc(-c5cc(-c6ccccc6)cc(-c6ccccc6)c5)n4)c3)n2)cc1]

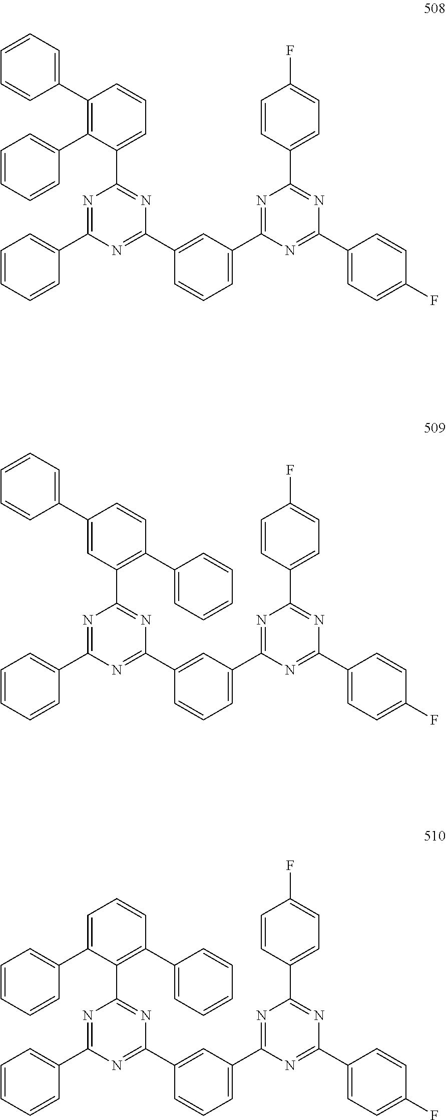 [SMILES: Fc1ccc(-c2nc(-c3ccc(F)cc3)nc(-c3cccc(-c4nc(-c5ccccc5)nc(-c5cccc(-c6ccccc6)c5-c5ccccc5)n4)c3)n2)cc1 ; Fc1ccc(-c2nc(-c3ccc(F)cc3)nc(-c3cccc(-c4nc(-c5ccccc5)nc(-c5cc(-c6ccccc6)ccc5-c5ccccc5)n4)c3)n2)cc1 ; Fc1ccc(-c2nc(-c3ccc(F)cc3)nc(-c3cccc(-c4nc(-c5ccccc5)nc(-c5c(-c6ccccc6)cccc5-c5ccccc5)n4)c3)n2)cc1]

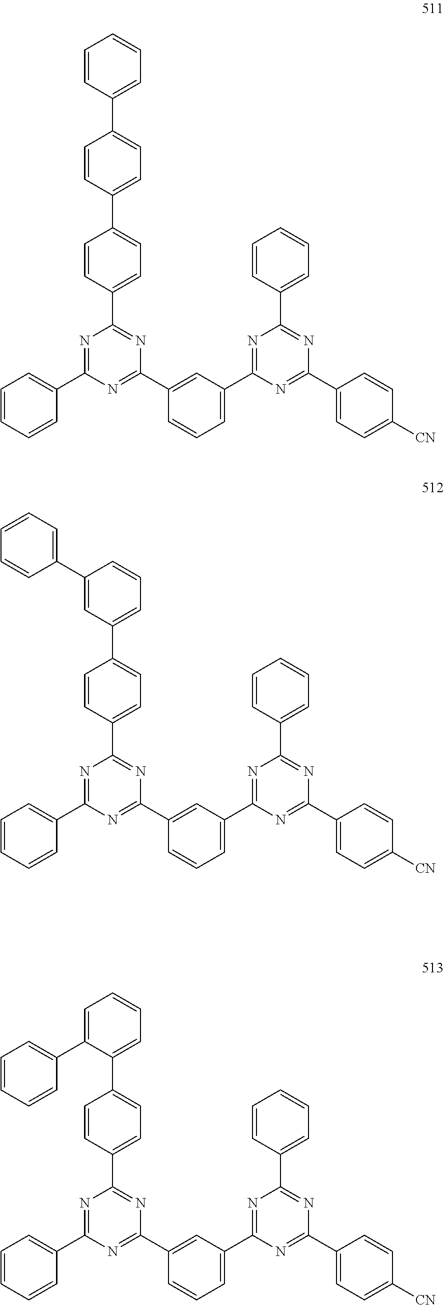 [SMILES: N#Cc1ccc(-c2nc(-c3ccccc3)nc(-c3cccc(-c4nc(-c5ccccc5)nc(-c5ccc(-c6ccc(-c7ccccc7)cc6)cc5)n4)c3)n2)cc1 ; N#Cc1ccc(-c2nc(-c3ccccc3)nc(-c3cccc(-c4nc(-c5ccccc5)nc(-c5ccc(-c6cccc(-c7ccccc7)c6)cc5)n4)c3)n2)cc1 ; N#Cc1ccc(-c2nc(-c3ccccc3)nc(-c3cccc(-c4nc(-c5ccccc5)nc(-c5ccc(-c6ccccc6-c6ccccc6)cc5)n4)c3)n2)cc1]

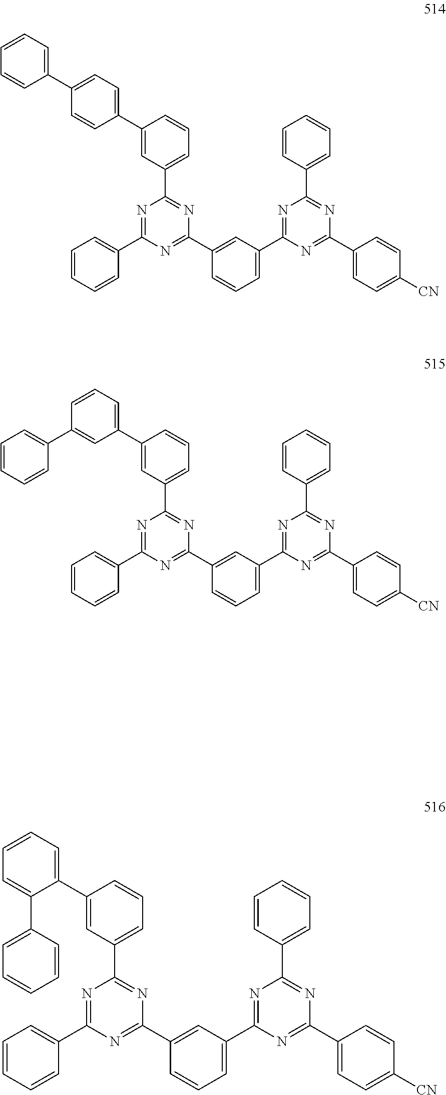 [SMILES: N#Cc1ccc(-c2nc(-c3ccccc3)nc(-c3cccc(-c4nc(-c5ccccc5)nc(-c5cccc(-c6ccc(-c7ccccc7)cc6)c5)n4)c3)n2)cc1 ; N#Cc1ccc(-c2nc(-c3ccccc3)nc(-c3cccc(-c4nc(-c5ccccc5)nc(-c5cccc(-c6cccc(-c7ccccc7)c6)c5)n4)c3)n2)cc1 ; N#Cc1ccc(-c2nc(-c3ccccc3)nc(-c3cccc(-c4nc(-c5ccccc5)nc(-c5cccc(-c6ccccc6-c6ccccc6)c5)n4)c3)n2)cc1]

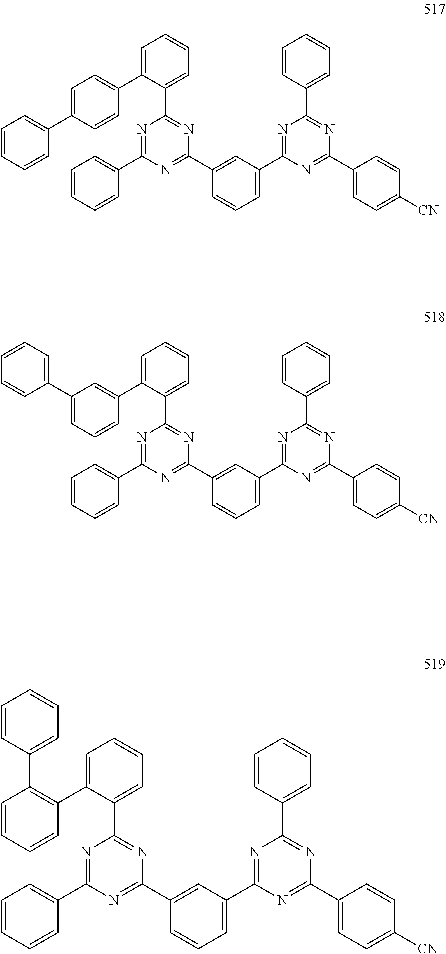 [SMILES: N#Cc1ccc(-c2nc(-c3ccccc3)nc(-c3cccc(-c4nc(-c5ccccc5)nc(-c5ccccc5-c5ccc(-c6ccccc6)cc5)n4)c3)n2)cc1 ; N#Cc1ccc(-c2nc(-c3ccccc3)nc(-c3cccc(-c4nc(-c5ccccc5)nc(-c5ccccc5-c5cccc(-c6ccccc6)c5)n4)c3)n2)cc1 ; N#Cc1ccc(-c2nc(-c3ccccc3)nc(-c3cccc(-c4nc(-c5ccccc5)nc(-c5ccccc5-c5ccccc5-c5ccccc5)n4)c3)n2)cc1]

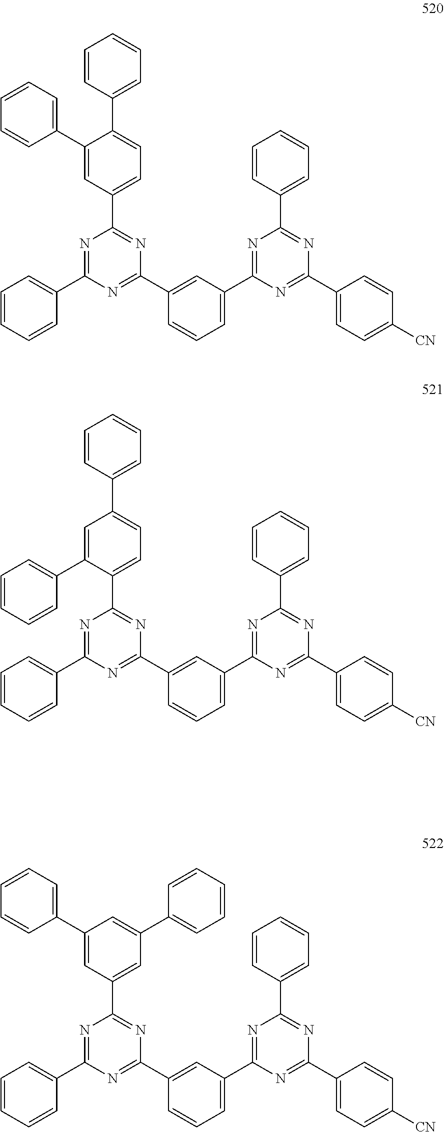 [SMILES: N#Cc1ccc(-c2nc(-c3ccccc3)nc(-c3cccc(-c4nc(-c5ccccc5)nc(-c5ccc(-c6ccccc6)c(-c6ccccc6)c5)n4)c3)n2)cc1 ; N#Cc1ccc(-c2nc(-c3ccccc3)nc(-c3cccc(-c4nc(-c5ccccc5)nc(-c5ccc(-c6ccccc6)cc5-c5ccccc5)n4)c3)n2)cc1 ; N#Cc1ccc(-c2nc(-c3ccccc3)nc(-c3cccc(-c4nc(-c5ccccc5)nc(-c5cc(-c6ccccc6)cc(-c6ccccc6)c5)n4)c3)n2)cc1]

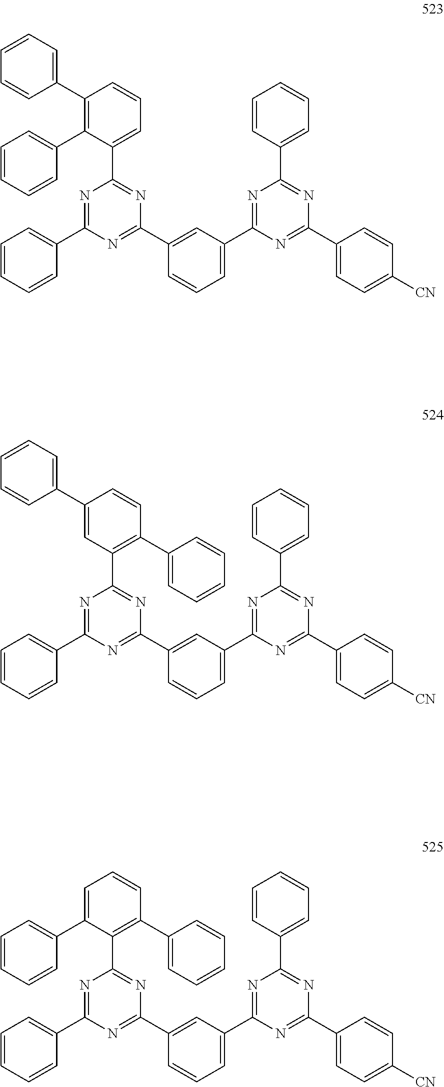 [SMILES: N#Cc1ccc(-c2nc(-c3ccccc3)nc(-c3cccc(-c4nc(-c5ccccc5)nc(-c5cccc(-c6ccccc6)c5-c5ccccc5)n4)c3)n2)cc1 ; N#Cc1ccc(-c2nc(-c3ccccc3)nc(-c3cccc(-c4nc(-c5ccccc5)nc(-c5cc(-c6ccccc6)ccc5-c5ccccc5)n4)c3)n2)cc1 ; N#Cc1ccc(-c2nc(-c3ccccc3)nc(-c3cccc(-c4nc(-c5ccccc5)nc(-c5c(-c6ccccc6)cccc5-c5ccccc5)n4)c3)n2)cc1]

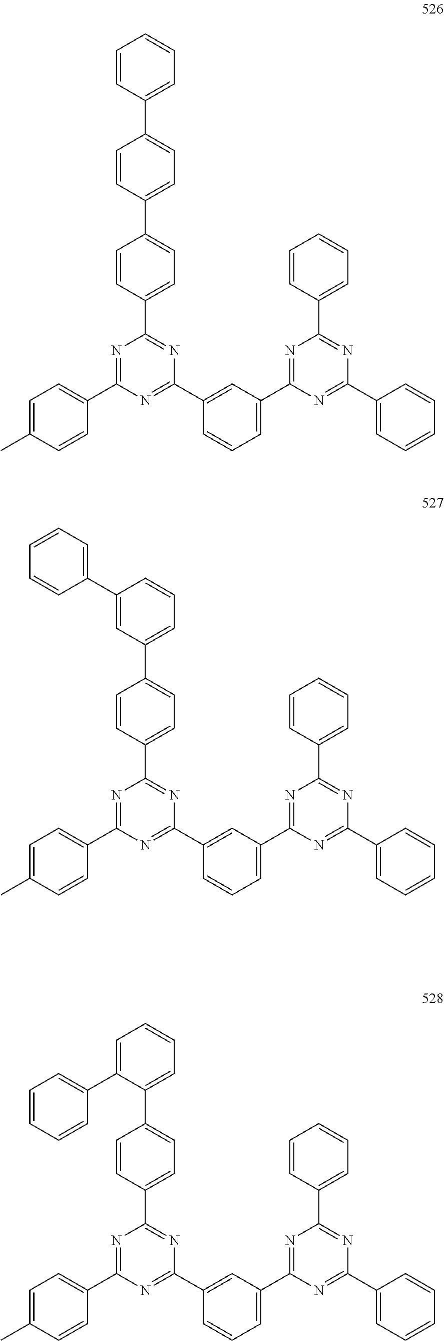 [SMILES: Cc1ccc(-c2nc(-c3ccc(-c4ccc(-c5ccccc5)cc4)cc3)nc(-c3cccc(-c4nc(-c5ccccc5)nc(-c5ccccc5)n4)c3)n2)cc1 ; Cc1ccc(-c2nc(-c3ccc(-c4cccc(-c5ccccc5)c4)cc3)nc(-c3cccc(-c4nc(-c5ccccc5)nc(-c5ccccc5)n4)c3)n2)cc1 ; Cc1ccc(-c2nc(-c3ccc(-c4ccccc4-c4ccccc4)cc3)nc(-c3cccc(-c4nc(-c5ccccc5)nc(-c5ccccc5)n4)c3)n2)cc1]

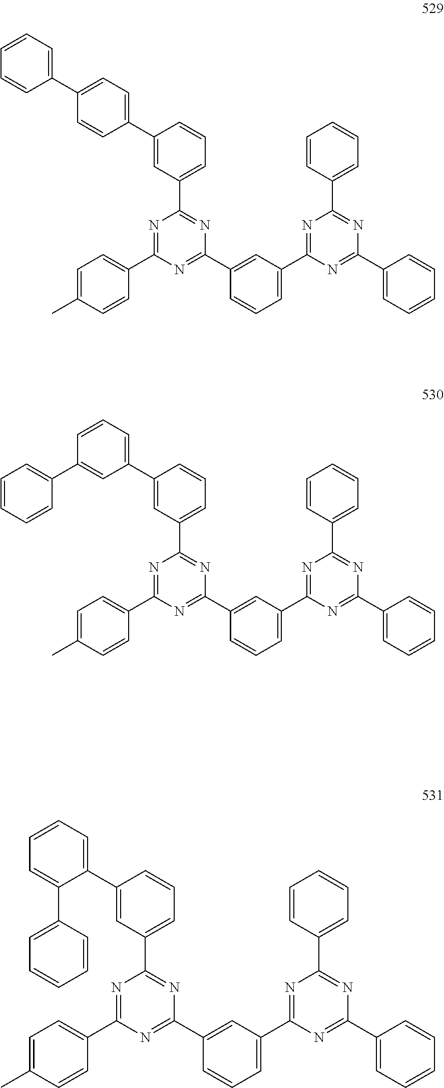 [SMILES: Cc1ccc(-c2nc(-c3cccc(-c4ccc(-c5ccccc5)cc4)c3)nc(-c3cccc(-c4nc(-c5ccccc5)nc(-c5ccccc5)n4)c3)n2)cc1 ; Cc1ccc(-c2nc(-c3cccc(-c4cccc(-c5ccccc5)c4)c3)nc(-c3cccc(-c4nc(-c5ccccc5)nc(-c5ccccc5)n4)c3)n2)cc1 ; Cc1ccc(-c2nc(-c3cccc(-c4nc(-c5ccccc5)nc(-c5ccccc5)n4)c3)nc(-c3cccc(-c4ccccc4-c4ccccc4)c3)n2)cc1]

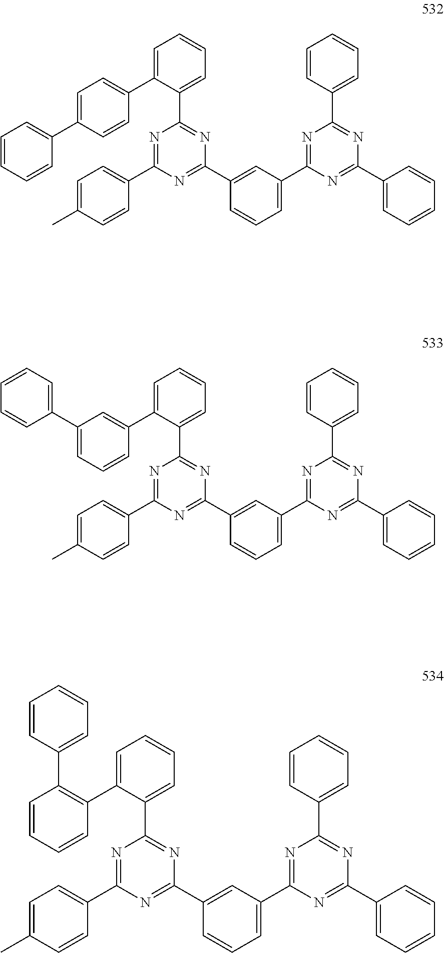 [SMILES: Cc1ccc(-c2nc(-c3cccc(-c4nc(-c5ccccc5)nc(-c5ccccc5)n4)c3)nc(-c3ccccc3-c3ccc(-c4ccccc4)cc3)n2)cc1 ; Cc1ccc(-c2nc(-c3cccc(-c4nc(-c5ccccc5)nc(-c5ccccc5)n4)c3)nc(-c3ccccc3-c3cccc(-c4ccccc4)c3)n2)cc1 ; Cc1ccc(-c2nc(-c3cccc(-c4nc(-c5ccccc5)nc(-c5ccccc5)n4)c3)nc(-c3ccccc3-c3ccccc3-c3ccccc3)n2)cc1]

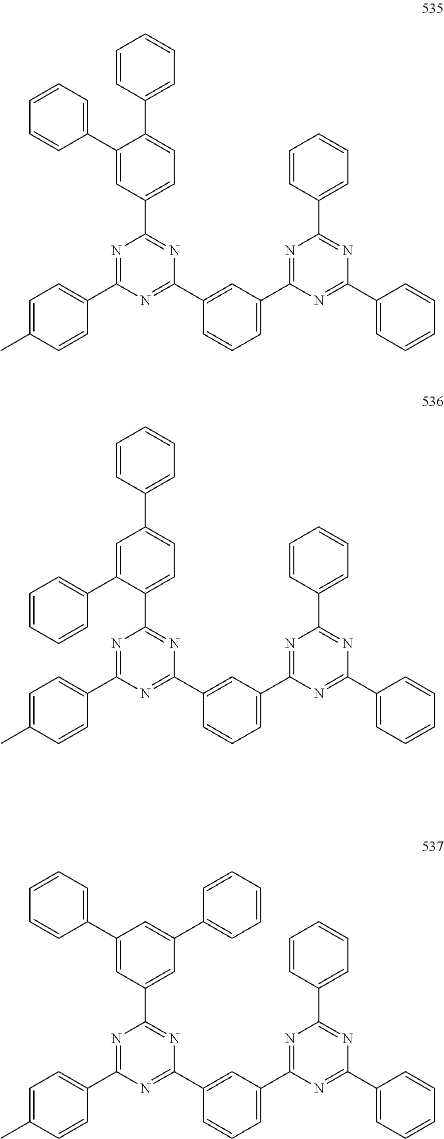 [SMILES: Cc1ccc(-c2nc(-c3cccc(-c4nc(-c5ccccc5)nc(-c5ccccc5)n4)c3)nc(-c3ccc(-c4ccccc4)c(-c4ccccc4)c3)n2)cc1 ; Cc1ccc(-c2nc(-c3cccc(-c4nc(-c5ccccc5)nc(-c5ccccc5)n4)c3)nc(-c3ccc(-c4ccccc4)cc3-c3ccccc3)n2)cc1 ; Cc1ccc(-c2nc(-c3cc(-c4ccccc4)cc(-c4ccccc4)c3)nc(-c3cccc(-c4nc(-c5ccccc5)nc(-c5ccccc5)n4)c3)n2)cc1]

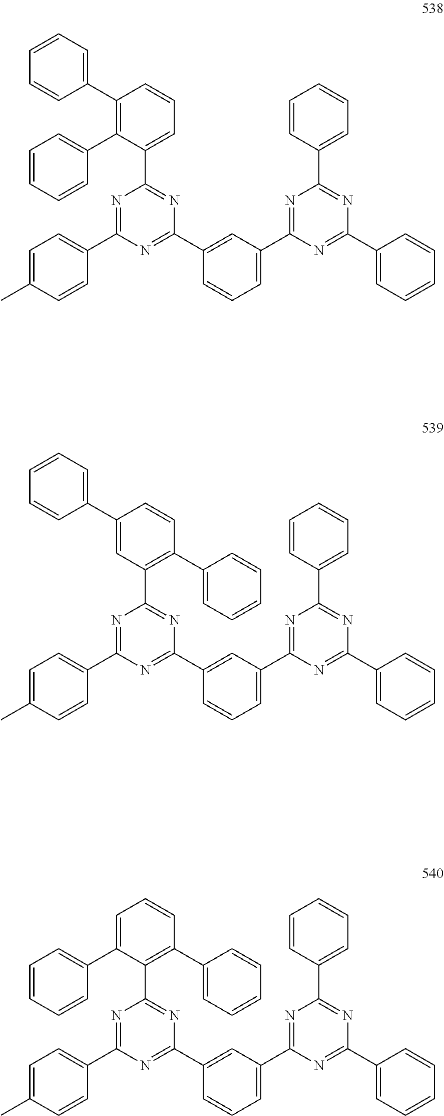 [SMILES: Cc1ccc(-c2nc(-c3cccc(-c4nc(-c5ccccc5)nc(-c5ccccc5)n4)c3)nc(-c3cccc(-c4ccccc4)c3-c3ccccc3)n2)cc1 ; Cc1ccc(-c2nc(-c3cccc(-c4nc(-c5ccccc5)nc(-c5ccccc5)n4)c3)nc(-c3cc(-c4ccccc4)ccc3-c3ccccc3)n2)cc1 ; Cc1ccc(-c2nc(-c3cccc(-c4nc(-c5ccccc5)nc(-c5ccccc5)n4)c3)nc(-c3c(-c4ccccc4)cccc3-c3ccccc3)n2)cc1]

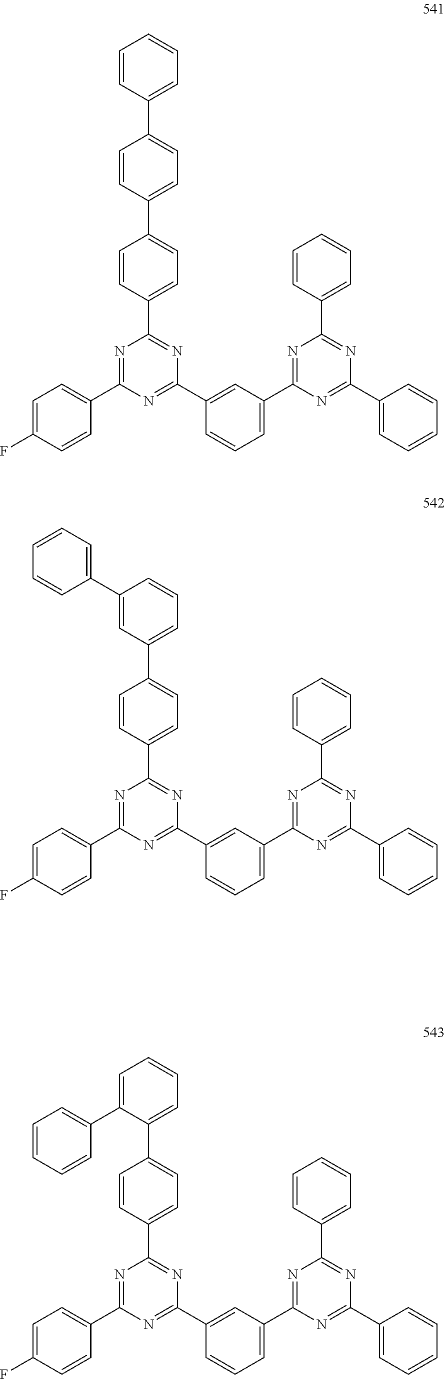 [SMILES: Fc1ccc(-c2nc(-c3ccc(-c4ccc(-c5ccccc5)cc4)cc3)nc(-c3cccc(-c4nc(-c5ccccc5)nc(-c5ccccc5)n4)c3)n2)cc1 ; Fc1ccc(-c2nc(-c3ccc(-c4cccc(-c5ccccc5)c4)cc3)nc(-c3cccc(-c4nc(-c5ccccc5)nc(-c5ccccc5)n4)c3)n2)cc1 ; Fc1ccc(-c2nc(-c3ccc(-c4ccccc4-c4ccccc4)cc3)nc(-c3cccc(-c4nc(-c5ccccc5)nc(-c5ccccc5)n4)c3)n2)cc1]

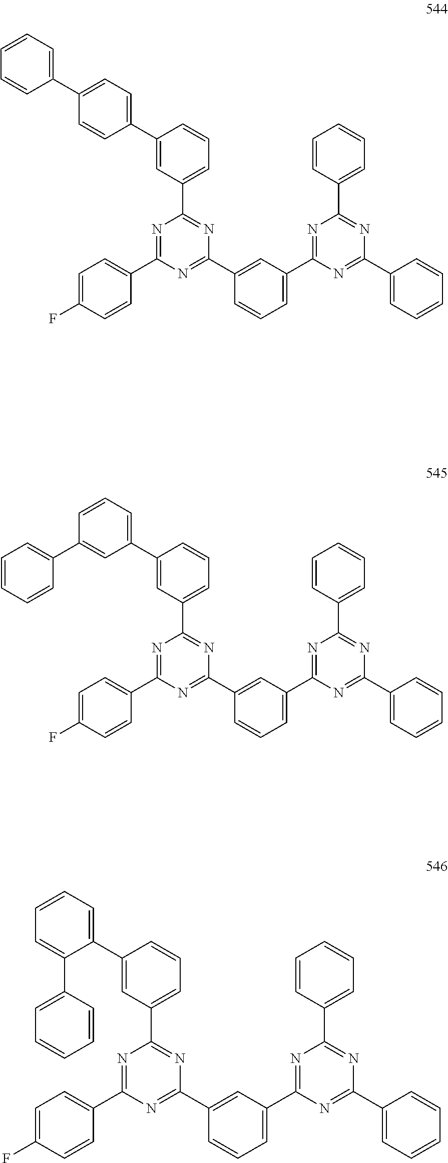 [SMILES: Fc1ccc(-c2nc(-c3cccc(-c4ccc(-c5ccccc5)cc4)c3)nc(-c3cccc(-c4nc(-c5ccccc5)nc(-c5ccccc5)n4)c3)n2)cc1 ; Fc1ccc(-c2nc(-c3cccc(-c4cccc(-c5ccccc5)c4)c3)nc(-c3cccc(-c4nc(-c5ccccc5)nc(-c5ccccc5)n4)c3)n2)cc1 ; Fc1ccc(-c2nc(-c3cccc(-c4nc(-c5ccccc5)nc(-c5ccccc5)n4)c3)nc(-c3cccc(-c4ccccc4-c4ccccc4)c3)n2)cc1]

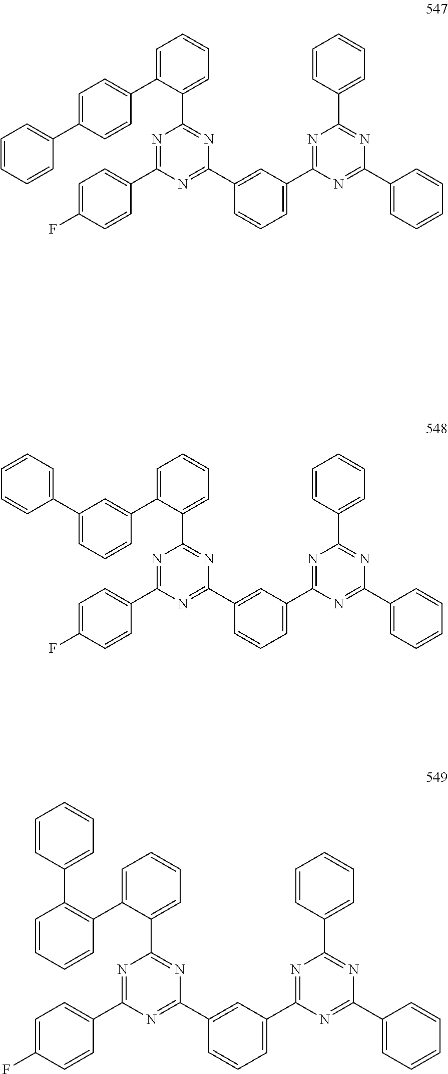 [SMILES: Fc1ccc(-c2nc(-c3cccc(-c4nc(-c5ccccc5)nc(-c5ccccc5)n4)c3)nc(-c3ccccc3-c3ccc(-c4ccccc4)cc3)n2)cc1 ; Fc1ccc(-c2nc(-c3cccc(-c4nc(-c5ccccc5)nc(-c5ccccc5)n4)c3)nc(-c3ccccc3-c3cccc(-c4ccccc4)c3)n2)cc1 ; Fc1ccc(-c2nc(-c3cccc(-c4nc(-c5ccccc5)nc(-c5ccccc5)n4)c3)nc(-c3ccccc3-c3ccccc3-c3ccccc3)n2)cc1]

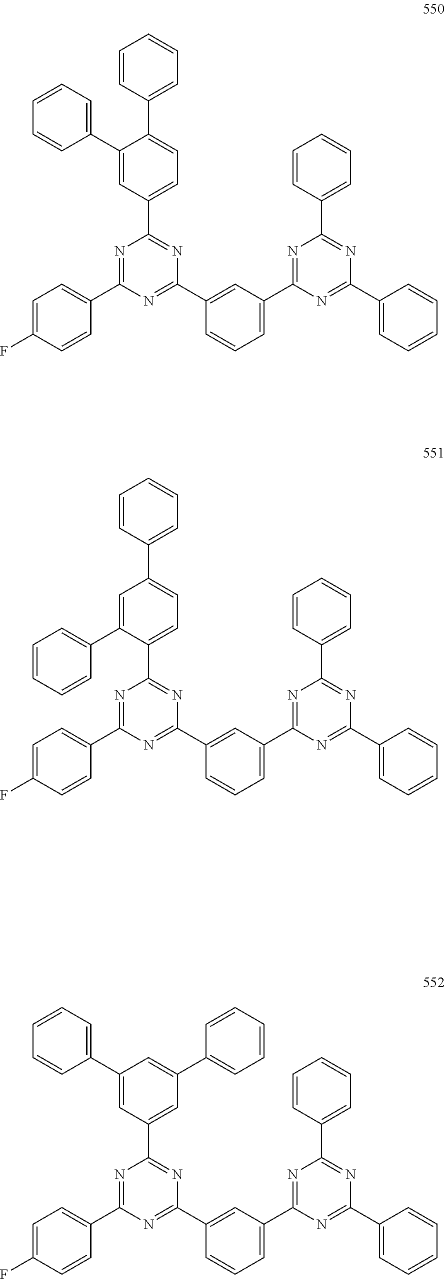 [SMILES: Fc1ccc(-c2nc(-c3cccc(-c4nc(-c5ccccc5)nc(-c5ccccc5)n4)c3)nc(-c3ccc(-c4ccccc4)c(-c4ccccc4)c3)n2)cc1 ; Fc1ccc(-c2nc(-c3cccc(-c4nc(-c5ccccc5)nc(-c5ccccc5)n4)c3)nc(-c3ccc(-c4ccccc4)cc3-c3ccccc3)n2)cc1 ; Fc1ccc(-c2nc(-c3cc(-c4ccccc4)cc(-c4ccccc4)c3)nc(-c3cccc(-c4nc(-c5ccccc5)nc(-c5ccccc5)n4)c3)n2)cc1]

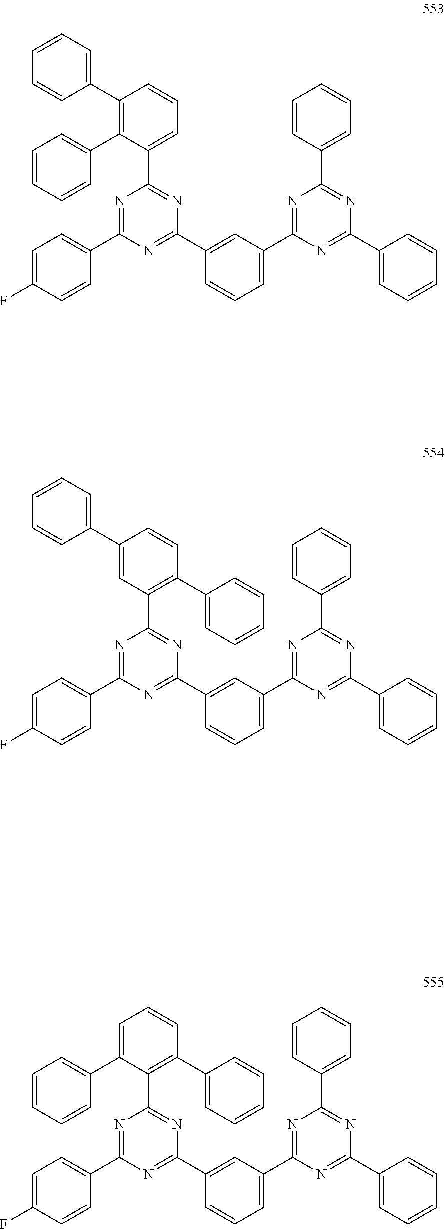 [SMILES: Fc1ccc(-c2nc(-c3cccc(-c4nc(-c5ccccc5)nc(-c5ccccc5)n4)c3)nc(-c3cccc(-c4ccccc4)c3-c3ccccc3)n2)cc1 ; Fc1ccc(-c2nc(-c3cccc(-c4nc(-c5ccccc5)nc(-c5ccccc5)n4)c3)nc(-c3cc(-c4ccccc4)ccc3-c3ccccc3)n2)cc1 ; Fc1ccc(-c2nc(-c3cccc(-c4nc(-c5ccccc5)nc(-c5ccccc5)n4)c3)nc(-c3c(-c4ccccc4)cccc3-c3ccccc3)n2)cc1]

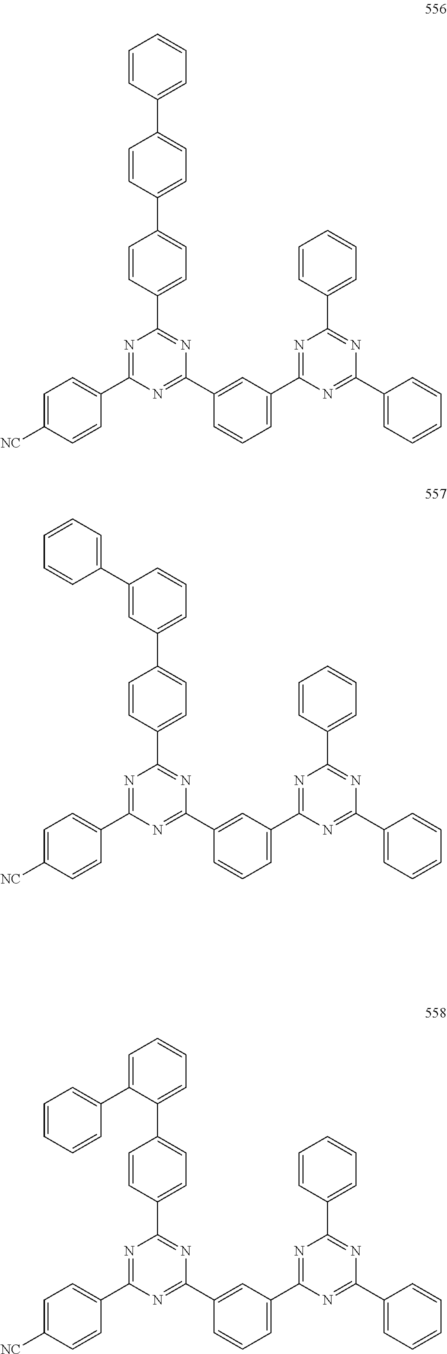 [SMILES: N#Cc1ccc(-c2nc(-c3ccc(-c4ccc(-c5ccccc5)cc4)cc3)nc(-c3cccc(-c4nc(-c5ccccc5)nc(-c5ccccc5)n4)c3)n2)cc1 ; N#Cc1ccc(-c2nc(-c3ccc(-c4cccc(-c5ccccc5)c4)cc3)nc(-c3cccc(-c4nc(-c5ccccc5)nc(-c5ccccc5)n4)c3)n2)cc1 ; N#Cc1ccc(-c2nc(-c3ccc(-c4ccccc4-c4ccccc4)cc3)nc(-c3cccc(-c4nc(-c5ccccc5)nc(-c5ccccc5)n4)c3)n2)cc1]

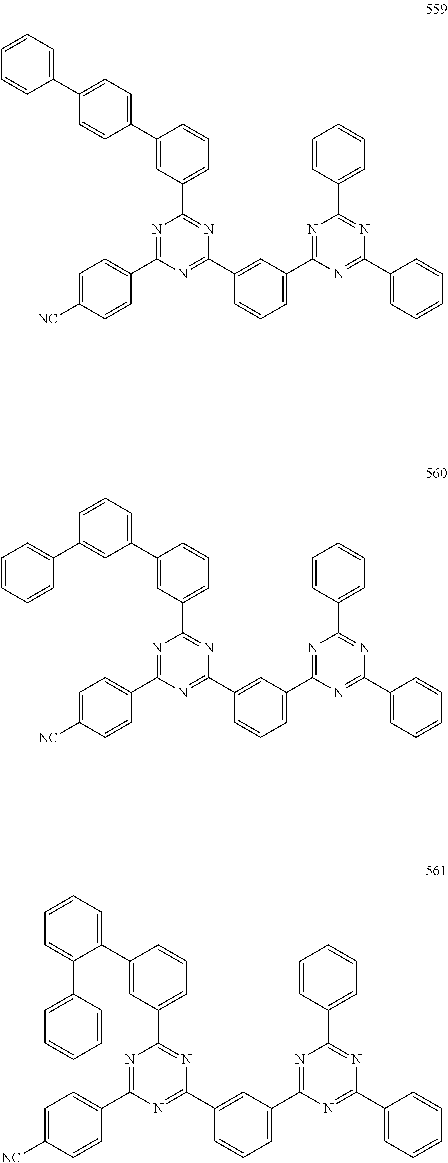 [SMILES: N#Cc1ccc(-c2nc(-c3cccc(-c4ccc(-c5ccccc5)cc4)c3)nc(-c3cccc(-c4nc(-c5ccccc5)nc(-c5ccccc5)n4)c3)n2)cc1 ; N#Cc1ccc(-c2nc(-c3cccc(-c4cccc(-c5ccccc5)c4)c3)nc(-c3cccc(-c4nc(-c5ccccc5)nc(-c5ccccc5)n4)c3)n2)cc1 ; N#Cc1ccc(-c2nc(-c3cccc(-c4nc(-c5ccccc5)nc(-c5ccccc5)n4)c3)nc(-c3cccc(-c4ccccc4-c4ccccc4)c3)n2)cc1]

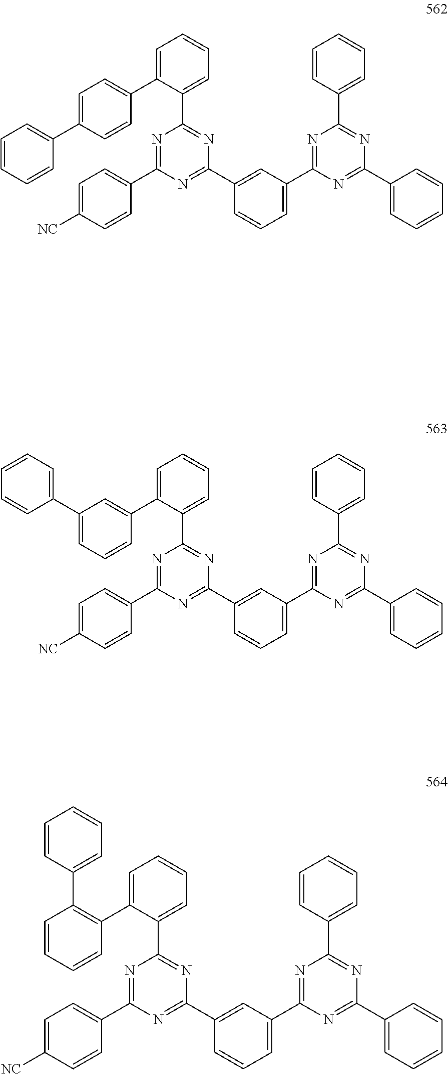 [SMILES: N#Cc1ccc(-c2nc(-c3cccc(-c4nc(-c5ccccc5)nc(-c5ccccc5)n4)c3)nc(-c3ccccc3-c3ccc(-c4ccccc4)cc3)n2)cc1 ; N#Cc1ccc(-c2nc(-c3cccc(-c4nc(-c5ccccc5)nc(-c5ccccc5)n4)c3)nc(-c3ccccc3-c3cccc(-c4ccccc4)c3)n2)cc1 ; N#Cc1ccc(-c2nc(-c3cccc(-c4nc(-c5ccccc5)nc(-c5ccccc5)n4)c3)nc(-c3ccccc3-c3ccccc3-c3ccccc3)n2)cc1]

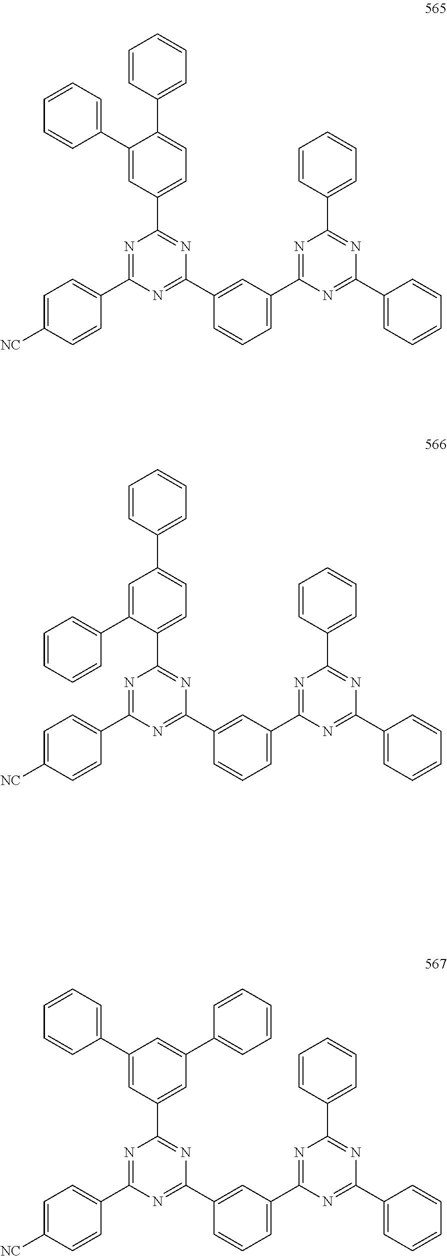 [SMILES: N#Cc1ccc(-c2nc(-c3cccc(-c4nc(-c5ccccc5)nc(-c5ccccc5)n4)c3)nc(-c3ccc(-c4ccccc4)c(-c4ccccc4)c3)n2)cc1 ; N#Cc1ccc(-c2nc(-c3cccc(-c4nc(-c5ccccc5)nc(-c5ccccc5)n4)c3)nc(-c3ccc(-c4ccccc4)cc3-c3ccccc3)n2)cc1 ; N#Cc1ccc(-c2nc(-c3cc(-c4ccccc4)cc(-c4ccccc4)c3)nc(-c3cccc(-c4nc(-c5ccccc5)nc(-c5ccccc5)n4)c3)n2)cc1]

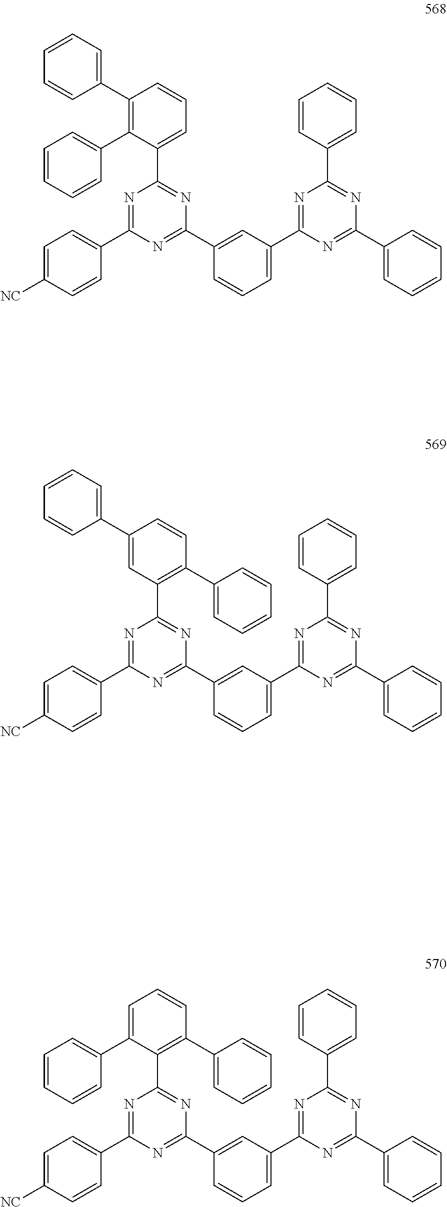 [SMILES: N#Cc1ccc(-c2nc(-c3cccc(-c4nc(-c5ccccc5)nc(-c5ccccc5)n4)c3)nc(-c3cccc(-c4ccccc4)c3-c3ccccc3)n2)cc1 ; N#Cc1ccc(-c2nc(-c3cccc(-c4nc(-c5ccccc5)nc(-c5ccccc5)n4)c3)nc(-c3cc(-c4ccccc4)ccc3-c3ccccc3)n2)cc1 ; N#Cc1ccc(-c2nc(-c3cccc(-c4nc(-c5ccccc5)nc(-c5ccccc5)n4)c3)nc(-c3c(-c4ccccc4)cccc3-c3ccccc3)n2)cc1]

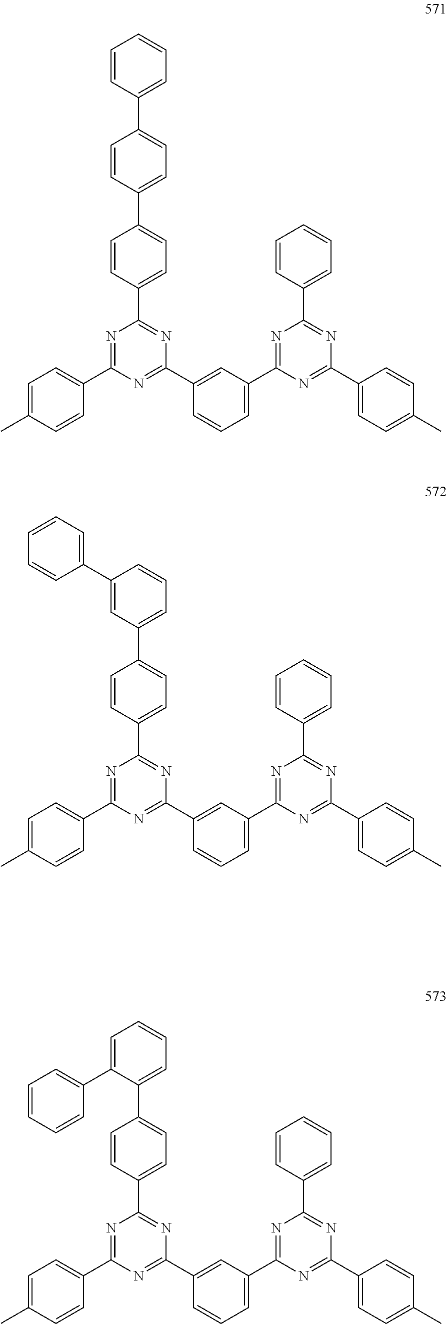

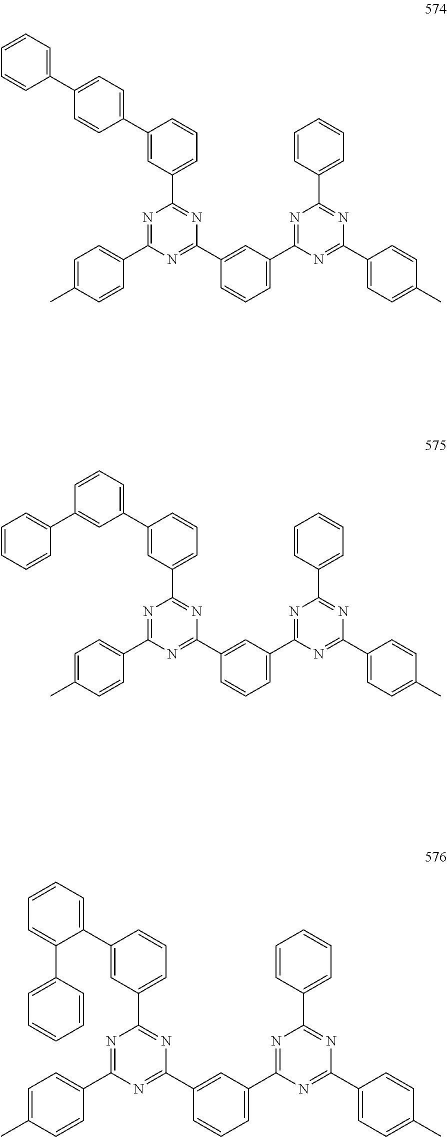

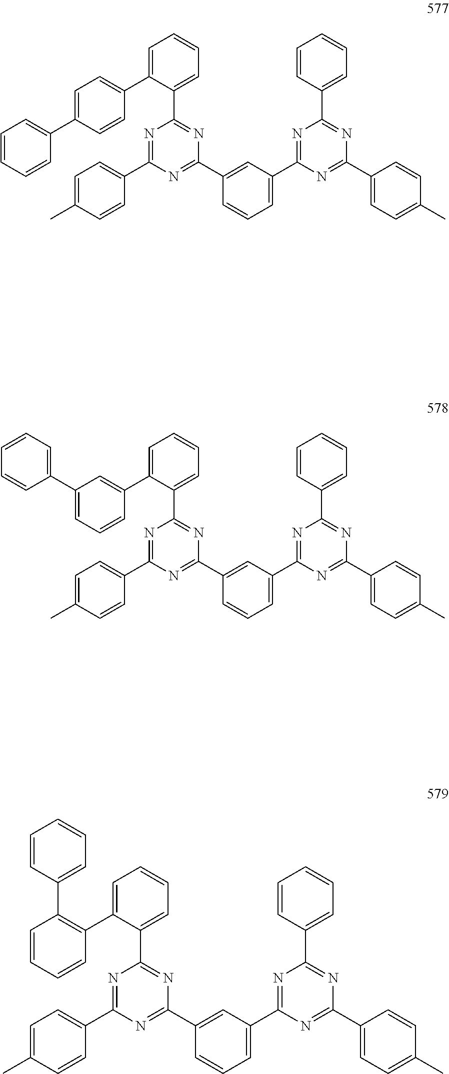

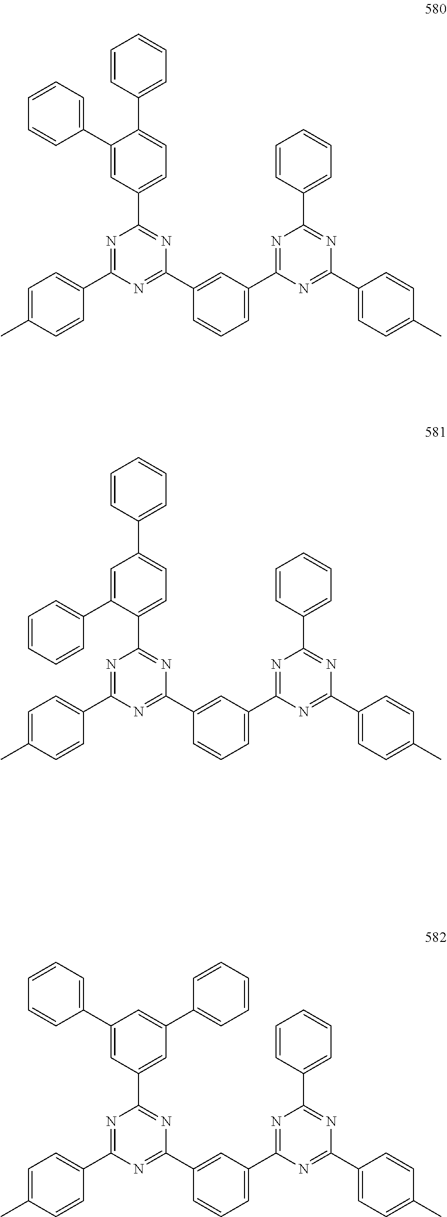

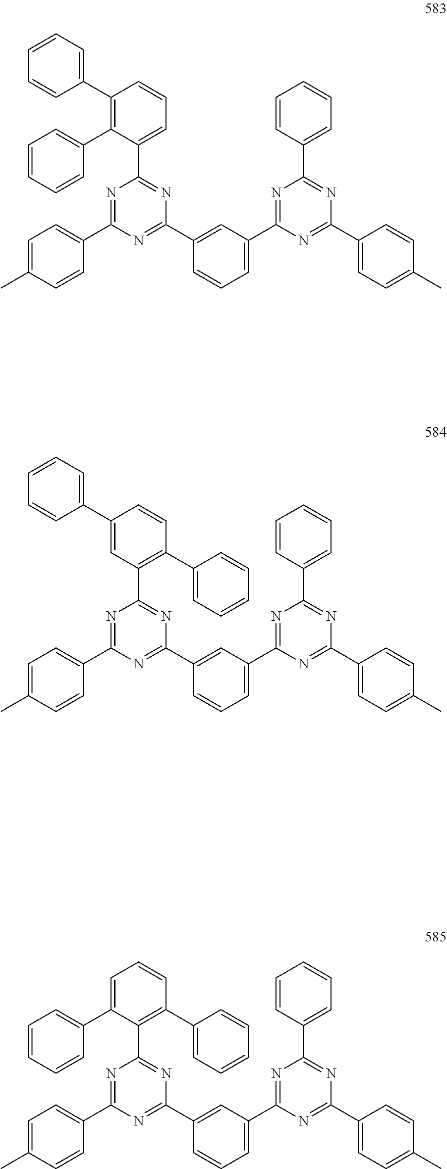

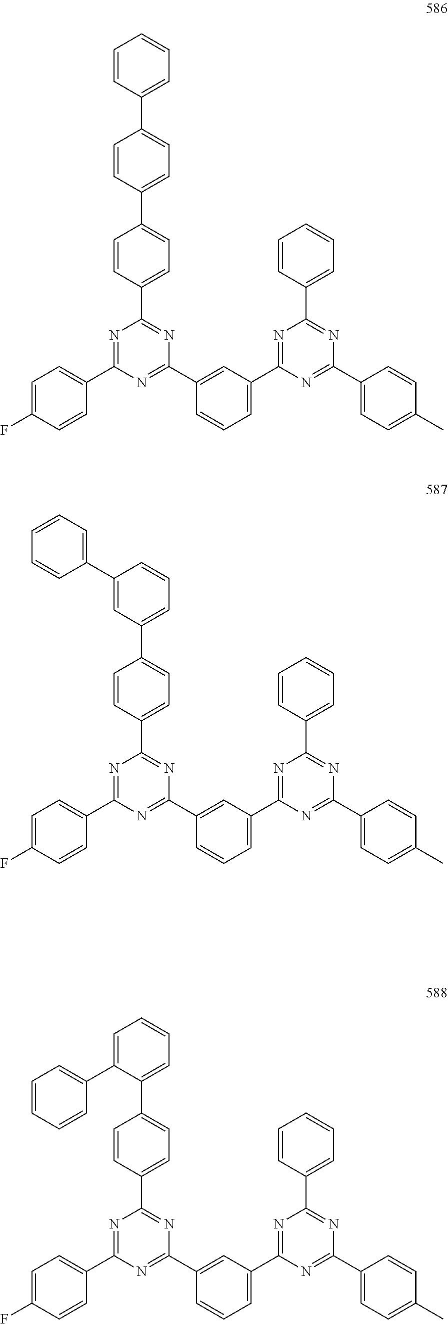 [SMILES: Cc1ccc(-c2nc(-c3ccccc3)nc(-c3cccc(-c4nc(-c5ccc(F)cc5)nc(-c5ccc(-c6ccc(-c7ccccc7)cc6)cc5)n4)c3)n2)cc1 ; Cc1ccc(-c2nc(-c3ccccc3)nc(-c3cccc(-c4nc(-c5ccc(F)cc5)nc(-c5ccc(-c6cccc(-c7ccccc7)c6)cc5)n4)c3)n2)cc1 ; Cc1ccc(-c2nc(-c3ccccc3)nc(-c3cccc(-c4nc(-c5ccc(F)cc5)nc(-c5ccc(-c6ccccc6-c6ccccc6)cc5)n4)c3)n2)cc1]

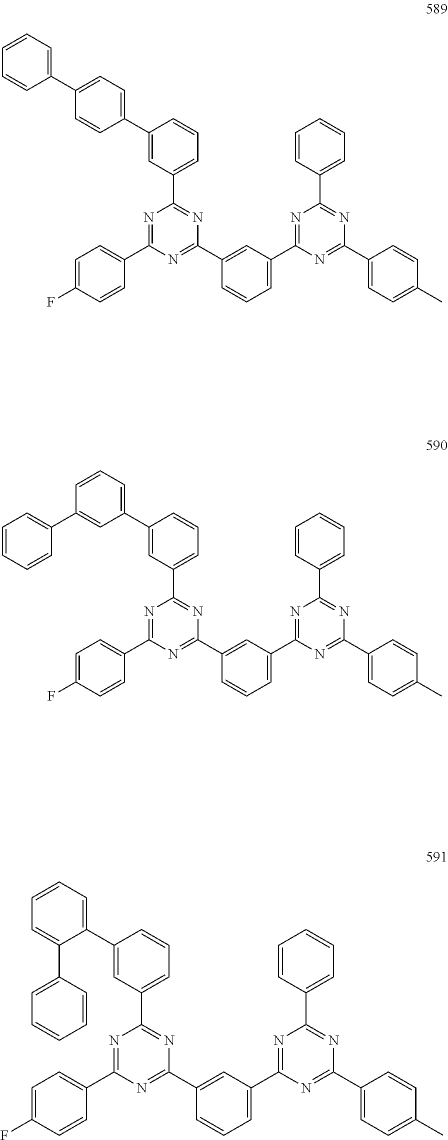 [SMILES: Cc1ccc(-c2nc(-c3ccccc3)nc(-c3cccc(-c4nc(-c5ccc(F)cc5)nc(-c5cccc(-c6ccc(-c7ccccc7)cc6)c5)n4)c3)n2)cc1 ; Cc1ccc(-c2nc(-c3ccccc3)nc(-c3cccc(-c4nc(-c5ccc(F)cc5)nc(-c5cccc(-c6cccc(-c7ccccc7)c6)c5)n4)c3)n2)cc1 ; Cc1ccc(-c2nc(-c3ccccc3)nc(-c3cccc(-c4nc(-c5ccc(F)cc5)nc(-c5cccc(-c6ccccc6-c6ccccc6)c5)n4)c3)n2)cc1]

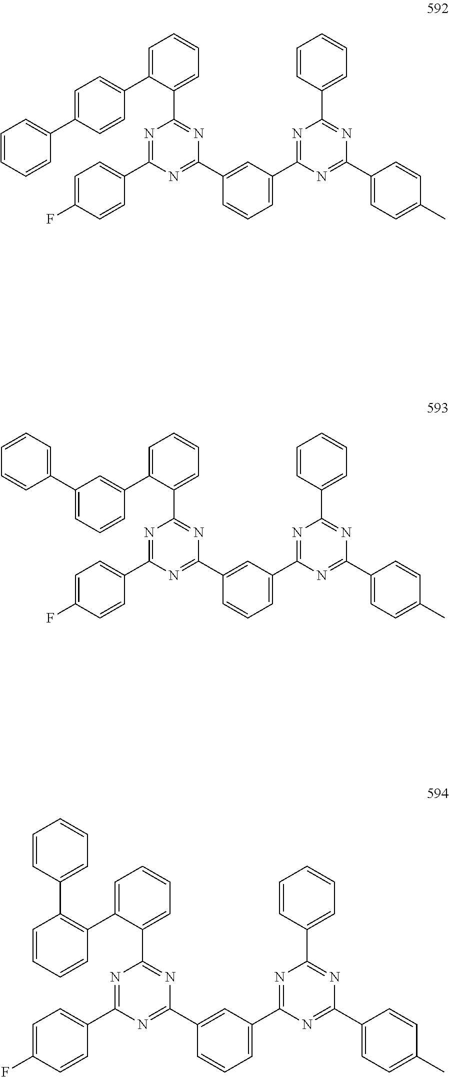 [SMILES: Cc1ccc(-c2nc(-c3ccccc3)nc(-c3cccc(-c4nc(-c5ccc(F)cc5)nc(-c5ccccc5-c5ccc(-c6ccccc6)cc5)n4)c3)n2)cc1 ; Cc1ccc(-c2nc(-c3ccccc3)nc(-c3cccc(-c4nc(-c5ccc(F)cc5)nc(-c5ccccc5-c5cccc(-c6ccccc6)c5)n4)c3)n2)cc1 ; Cc1ccc(-c2nc(-c3ccccc3)nc(-c3cccc(-c4nc(-c5ccc(F)cc5)nc(-c5ccccc5-c5ccccc5-c5ccccc5)n4)c3)n2)cc1]

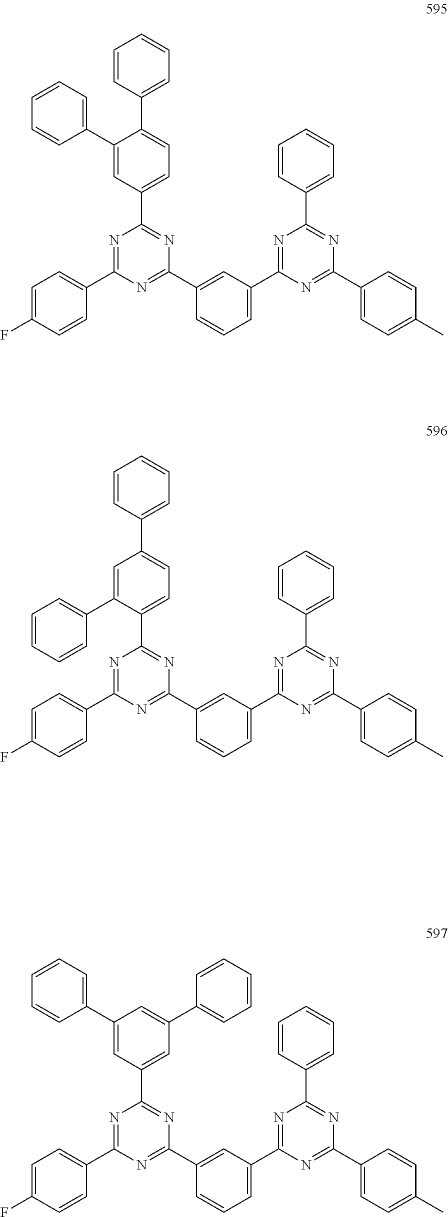 [SMILES: Cc1ccc(-c2nc(-c3ccccc3)nc(-c3cccc(-c4nc(-c5ccc(F)cc5)nc(-c5ccc(-c6ccccc6)c(-c6ccccc6)c5)n4)c3)n2)cc1 ; Cc1ccc(-c2nc(-c3ccccc3)nc(-c3cccc(-c4nc(-c5ccc(F)cc5)nc(-c5ccc(-c6ccccc6)cc5-c5ccccc5)n4)c3)n2)cc1 ; Cc1ccc(-c2nc(-c3ccccc3)nc(-c3cccc(-c4nc(-c5ccc(F)cc5)nc(-c5cc(-c6ccccc6)cc(-c6ccccc6)c5)n4)c3)n2)cc1]

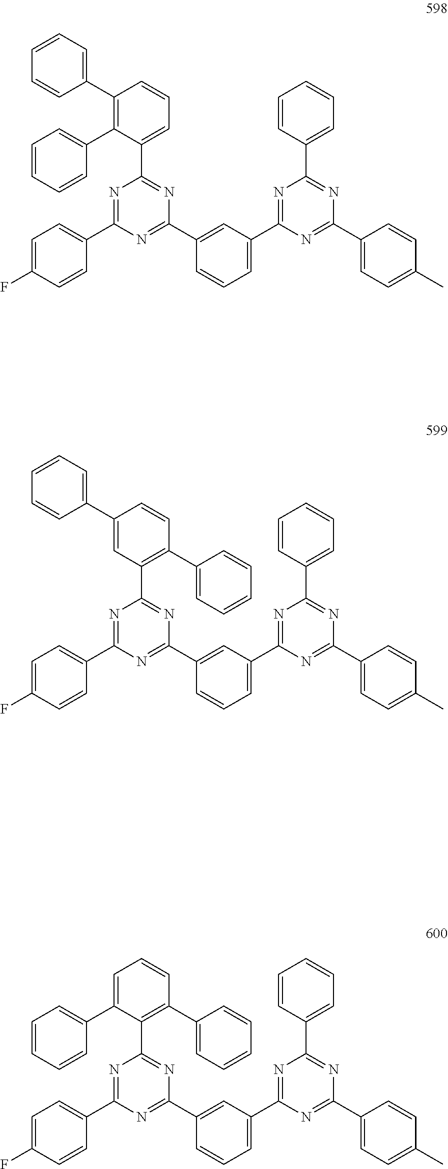 [SMILES: Cc1ccc(-c2nc(-c3ccccc3)nc(-c3cccc(-c4nc(-c5ccc(F)cc5)nc(-c5cccc(-c6ccccc6)c5-c5ccccc5)n4)c3)n2)cc1 ; Cc1ccc(-c2nc(-c3ccccc3)nc(-c3cccc(-c4nc(-c5ccc(F)cc5)nc(-c5cc(-c6ccccc6)ccc5-c5ccccc5)n4)c3)n2)cc1 ; Cc1ccc(-c2nc(-c3ccccc3)nc(-c3cccc(-c4nc(-c5ccc(F)cc5)nc(-c5c(-c6ccccc6)cccc5-c5ccccc5)n4)c3)n2)cc1]

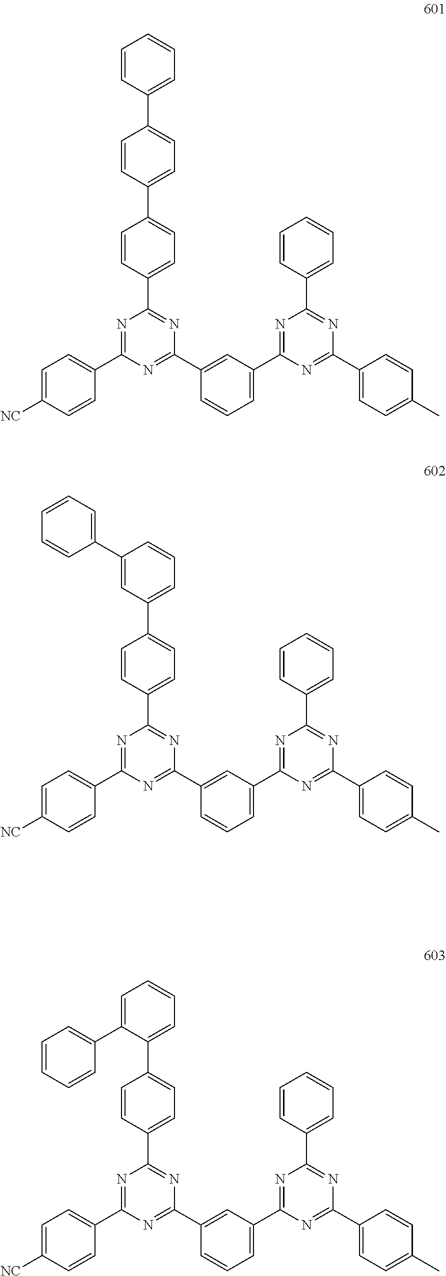 [SMILES: Cc1ccc(-c2nc(-c3ccccc3)nc(-c3cccc(-c4nc(-c5ccc(C#N)cc5)nc(-c5ccc(-c6ccc(-c7ccccc7)cc6)cc5)n4)c3)n2)cc1 ; Cc1ccc(-c2nc(-c3ccccc3)nc(-c3cccc(-c4nc(-c5ccc(C#N)cc5)nc(-c5ccc(-c6cccc(-c7ccccc7)c6)cc5)n4)c3)n2)cc1 ; Cc1ccc(-c2nc(-c3ccccc3)nc(-c3cccc(-c4nc(-c5ccc(C#N)cc5)nc(-c5ccc(-c6ccccc6-c6ccccc6)cc5)n4)c3)n2)cc1]

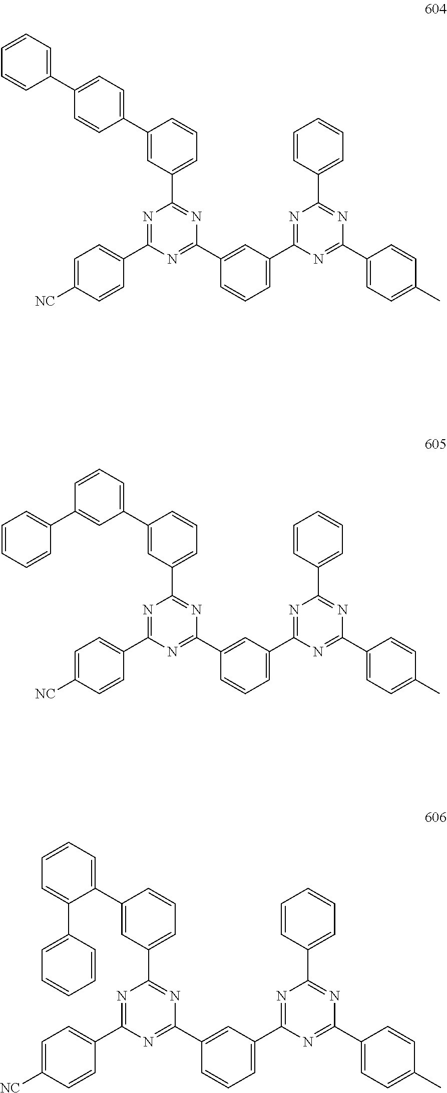 [SMILES: Cc1ccc(-c2nc(-c3ccccc3)nc(-c3cccc(-c4nc(-c5ccc(C#N)cc5)nc(-c5cccc(-c6ccc(-c7ccccc7)cc6)c5)n4)c3)n2)cc1 ; Cc1ccc(-c2nc(-c3ccccc3)nc(-c3cccc(-c4nc(-c5ccc(C#N)cc5)nc(-c5cccc(-c6cccc(-c7ccccc7)c6)c5)n4)c3)n2)cc1 ; Cc1ccc(-c2nc(-c3ccccc3)nc(-c3cccc(-c4nc(-c5ccc(C#N)cc5)nc(-c5cccc(-c6ccccc6-c6ccccc6)c5)n4)c3)n2)cc1]

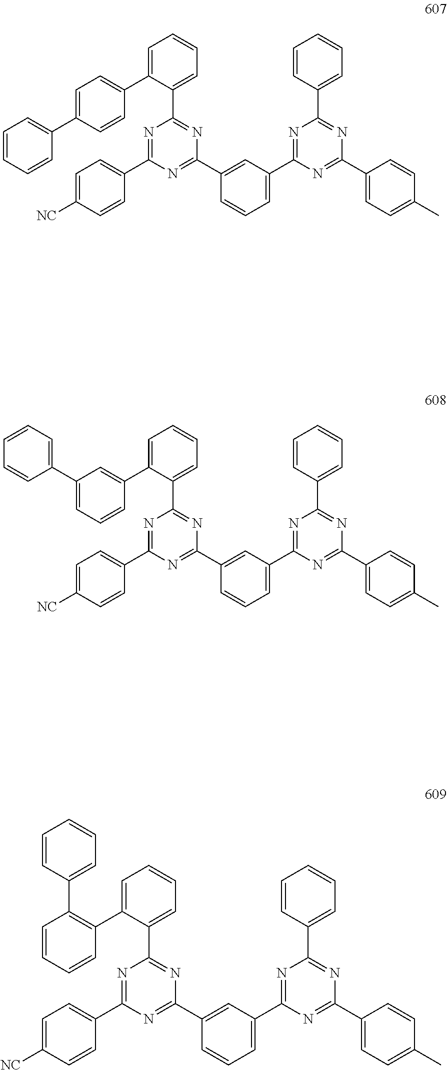 [SMILES: Cc1ccc(-c2nc(-c3ccccc3)nc(-c3cccc(-c4nc(-c5ccc(C#N)cc5)nc(-c5ccccc5-c5ccc(-c6ccccc6)cc5)n4)c3)n2)cc1 ; Cc1ccc(-c2nc(-c3ccccc3)nc(-c3cccc(-c4nc(-c5ccc(C#N)cc5)nc(-c5ccccc5-c5cccc(-c6ccccc6)c5)n4)c3)n2)cc1 ; Cc1ccc(-c2nc(-c3ccccc3)nc(-c3cccc(-c4nc(-c5ccc(C#N)cc5)nc(-c5ccccc5-c5ccccc5-c5ccccc5)n4)c3)n2)cc1]

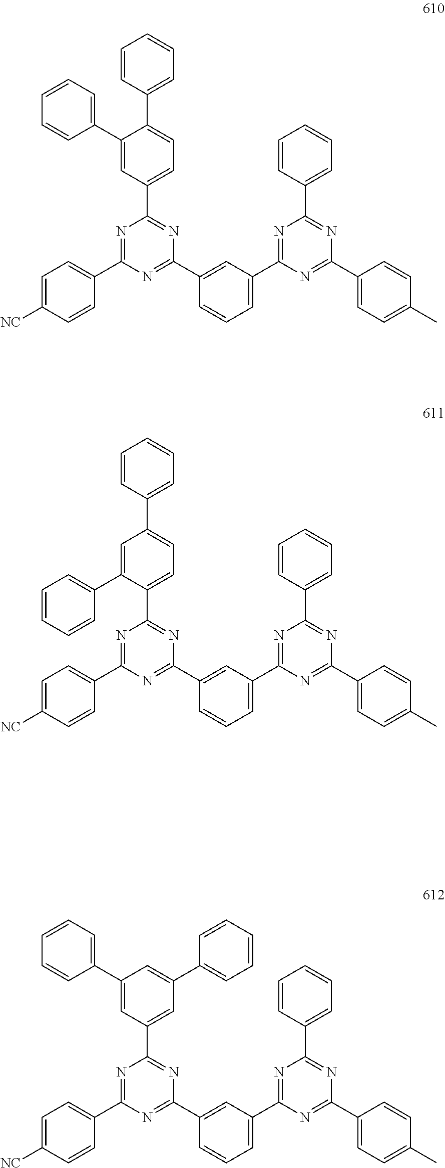 [SMILES: Cc1ccc(-c2nc(-c3ccccc3)nc(-c3cccc(-c4nc(-c5ccc(C#N)cc5)nc(-c5ccc(-c6ccccc6)c(-c6ccccc6)c5)n4)c3)n2)cc1 ; Cc1ccc(-c2nc(-c3ccccc3)nc(-c3cccc(-c4nc(-c5ccc(C#N)cc5)nc(-c5ccc(-c6ccccc6)cc5-c5ccccc5)n4)c3)n2)cc1 ; Cc1ccc(-c2nc(-c3ccccc3)nc(-c3cccc(-c4nc(-c5ccc(C#N)cc5)nc(-c5cc(-c6ccccc6)cc(-c6ccccc6)c5)n4)c3)n2)cc1]

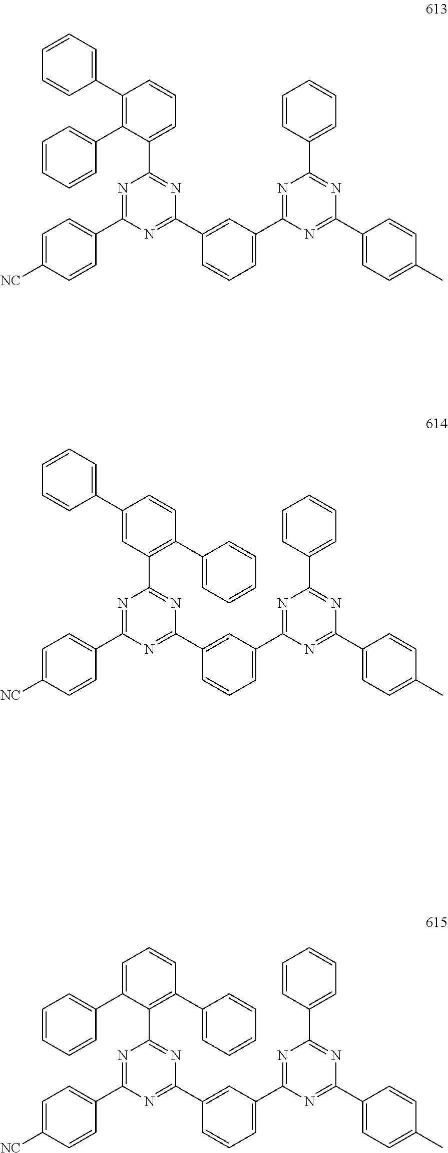 [SMILES: Cc1ccc(-c2nc(-c3ccccc3)nc(-c3cccc(-c4nc(-c5ccc(C#N)cc5)nc(-c5cccc(-c6ccccc6)c5-c5ccccc5)n4)c3)n2)cc1 ; Cc1ccc(-c2nc(-c3ccccc3)nc(-c3cccc(-c4nc(-c5ccc(C#N)cc5)nc(-c5cc(-c6ccccc6)ccc5-c5ccccc5)n4)c3)n2)cc1 ; Cc1ccc(-c2nc(-c3ccccc3)nc(-c3cccc(-c4nc(-c5ccc(C#N)cc5)nc(-c5c(-c6ccccc6)cccc5-c5ccccc5)n4)c3)n2)cc1]

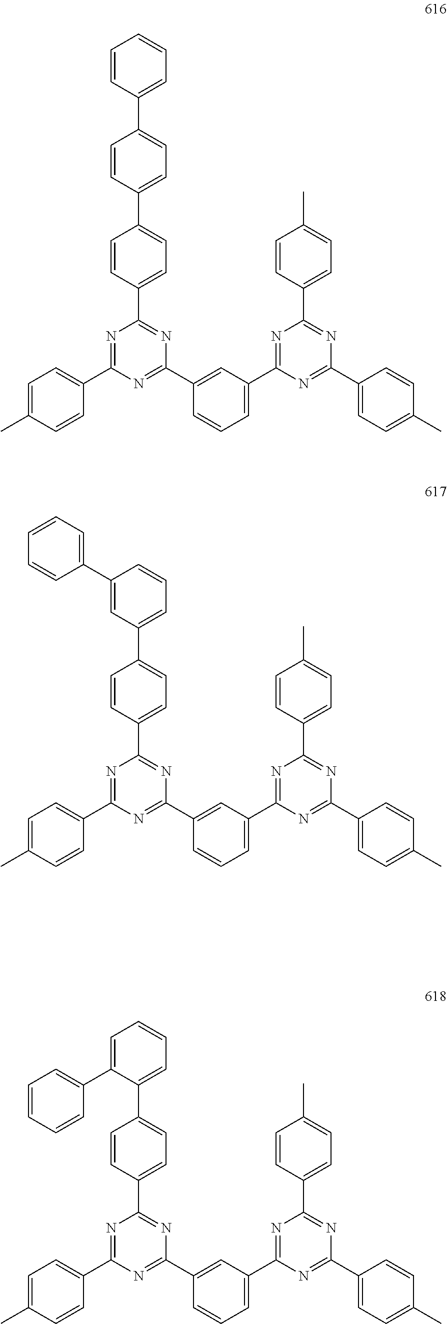

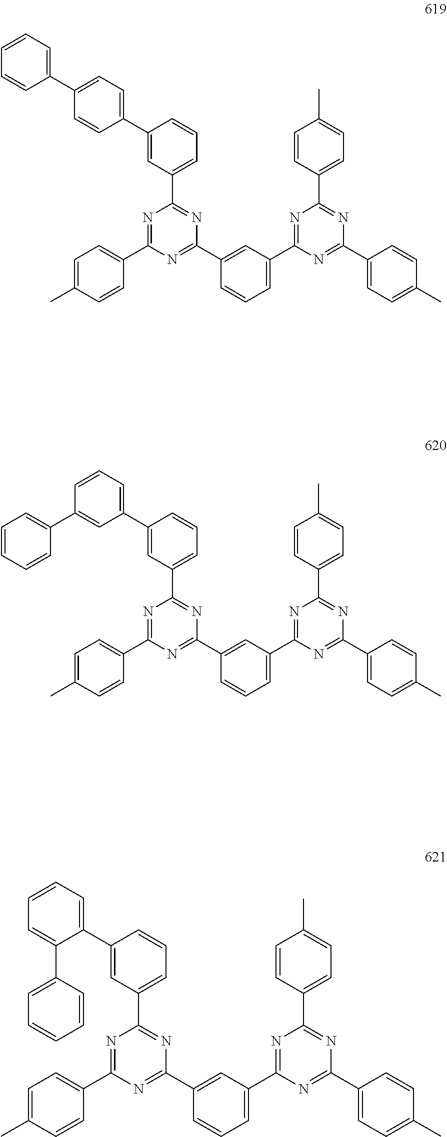

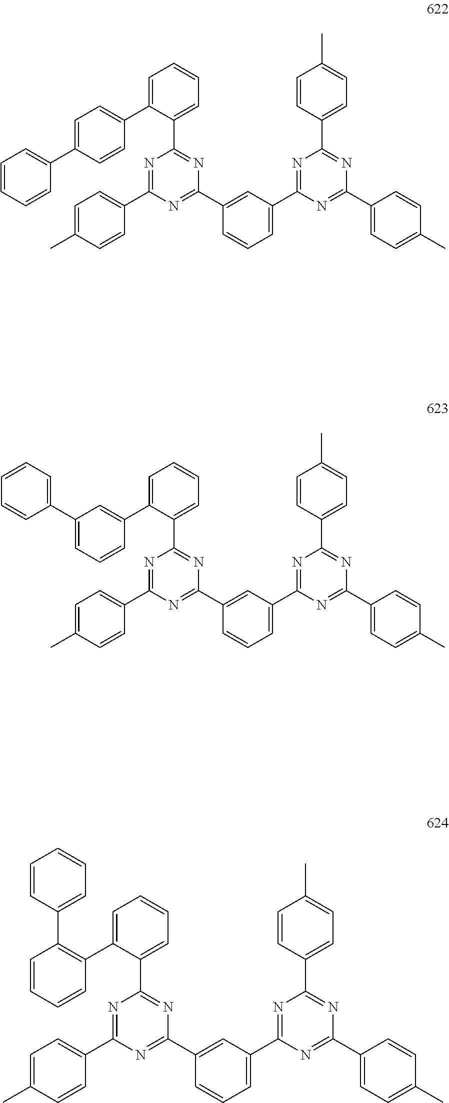

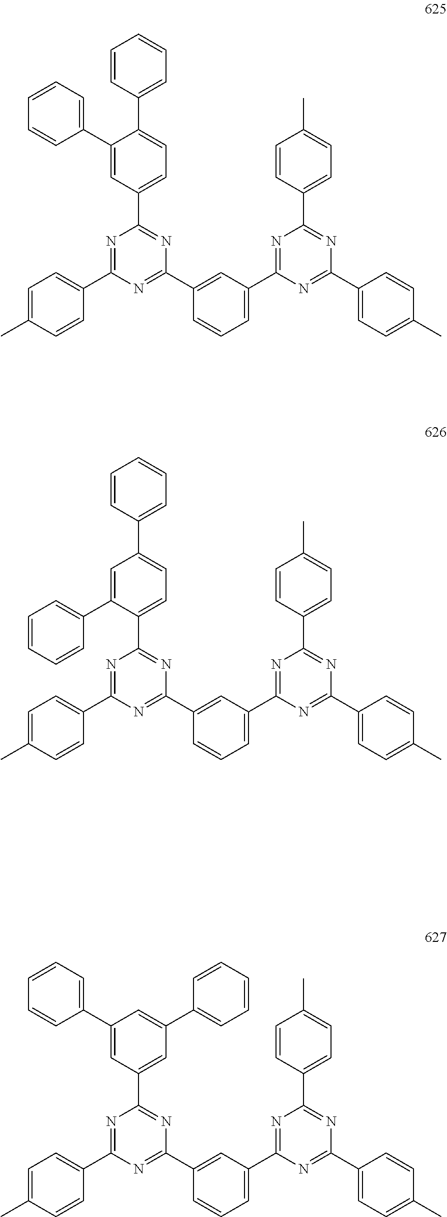

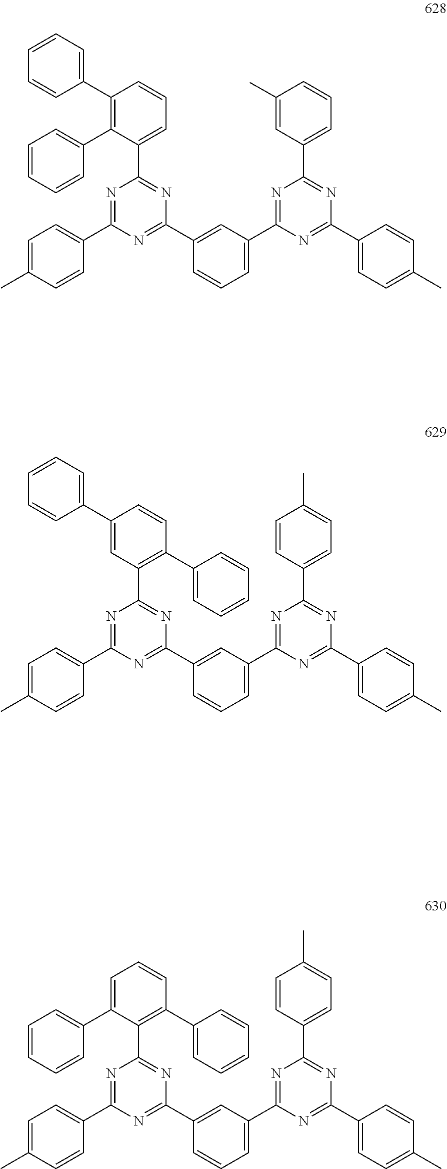

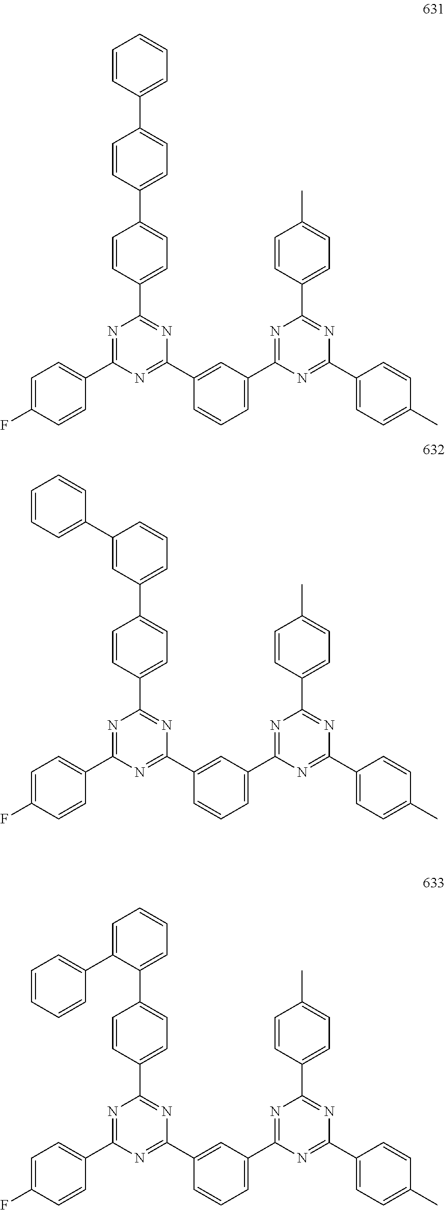 [SMILES: Cc1ccc(-c2nc(-c3ccc(C)cc3)nc(-c3cccc(-c4nc(-c5ccc(F)cc5)nc(-c5ccc(-c6ccc(-c7ccccc7)cc6)cc5)n4)c3)n2)cc1 ; Cc1ccc(-c2nc(-c3ccc(C)cc3)nc(-c3cccc(-c4nc(-c5ccc(F)cc5)nc(-c5ccc(-c6cccc(-c7ccccc7)c6)cc5)n4)c3)n2)cc1 ; Cc1ccc(-c2nc(-c3ccc(C)cc3)nc(-c3cccc(-c4nc(-c5ccc(F)cc5)nc(-c5ccc(-c6ccccc6-c6ccccc6)cc5)n4)c3)n2)cc1]

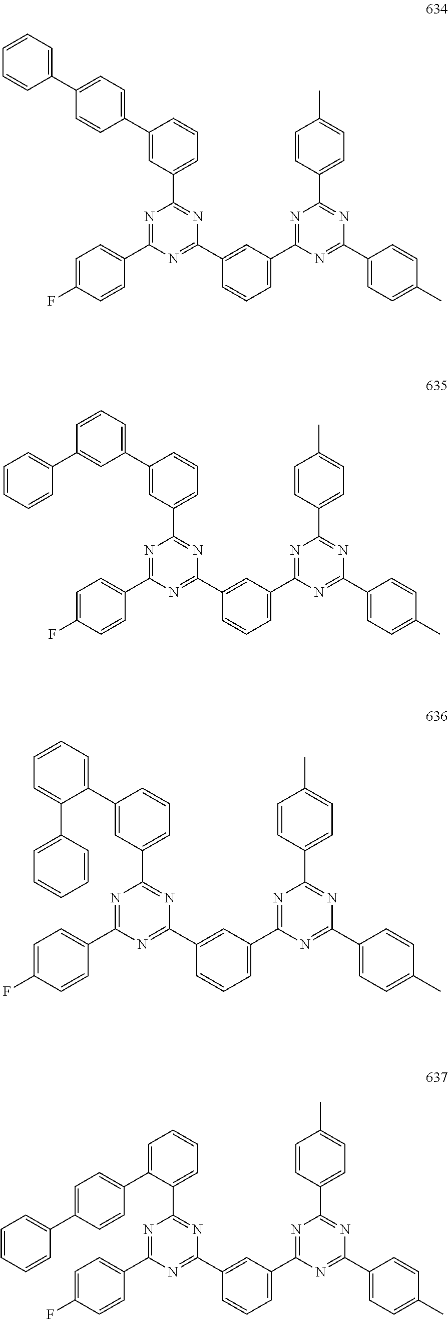 [SMILES: Cc1ccc(-c2nc(-c3ccc(C)cc3)nc(-c3cccc(-c4nc(-c5ccc(F)cc5)nc(-c5cccc(-c6ccc(-c7ccccc7)cc6)c5)n4)c3)n2)cc1 ; Cc1ccc(-c2nc(-c3ccc(C)cc3)nc(-c3cccc(-c4nc(-c5ccc(F)cc5)nc(-c5cccc(-c6cccc(-c7ccccc7)c6)c5)n4)c3)n2)cc1 ; Cc1ccc(-c2nc(-c3ccc(C)cc3)nc(-c3cccc(-c4nc(-c5ccc(F)cc5)nc(-c5cccc(-c6ccccc6-c6ccccc6)c5)n4)c3)n2)cc1 ; Cc1ccc(-c2nc(-c3ccc(C)cc3)nc(-c3cccc(-c4nc(-c5ccc(F)cc5)nc(-c5ccccc5-c5ccc(-c6ccccc6)cc5)n4)c3)n2)cc1]

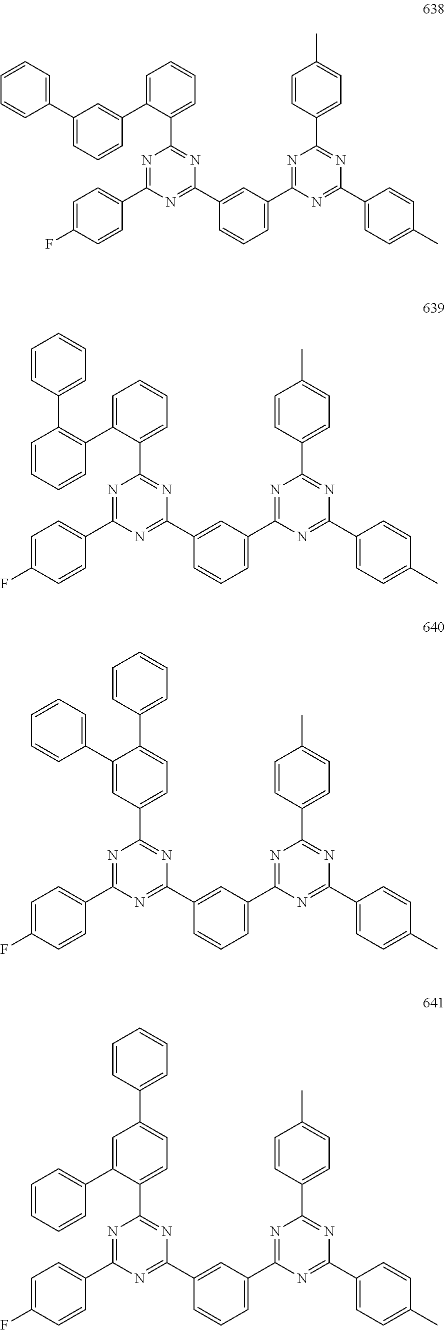 [SMILES: Cc1ccc(-c2nc(-c3ccc(C)cc3)nc(-c3cccc(-c4nc(-c5ccc(F)cc5)nc(-c5ccccc5-c5cccc(-c6ccccc6)c5)n4)c3)n2)cc1 ; Cc1ccc(-c2nc(-c3ccc(C)cc3)nc(-c3cccc(-c4nc(-c5ccc(F)cc5)nc(-c5ccccc5-c5ccccc5-c5ccccc5)n4)c3)n2)cc1 ; Cc1ccc(-c2nc(-c3ccc(C)cc3)nc(-c3cccc(-c4nc(-c5ccc(F)cc5)nc(-c5ccc(-c6ccccc6)c(-c6ccccc6)c5)n4)c3)n2)cc1 ; Cc1ccc(-c2nc(-c3ccc(C)cc3)nc(-c3cccc(-c4nc(-c5ccc(F)cc5)nc(-c5ccc(-c6ccccc6)cc5-c5ccccc5)n4)c3)n2)cc1]

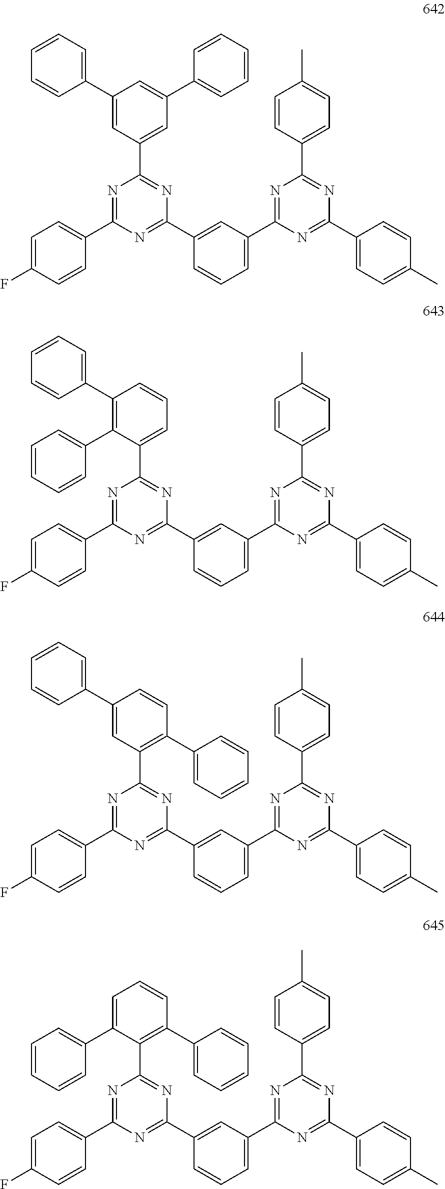 [SMILES: Cc1ccc(-c2nc(-c3ccc(C)cc3)nc(-c3cccc(-c4nc(-c5ccc(F)cc5)nc(-c5cc(-c6ccccc6)cc(-c6ccccc6)c5)n4)c3)n2)cc1 ; Cc1ccc(-c2nc(-c3ccc(C)cc3)nc(-c3cccc(-c4nc(-c5ccc(F)cc5)nc(-c5cccc(-c6ccccc6)c5-c5ccccc5)n4)c3)n2)cc1 ; Cc1ccc(-c2nc(-c3ccc(C)cc3)nc(-c3cccc(-c4nc(-c5ccc(F)cc5)nc(-c5cc(-c6ccccc6)ccc5-c5ccccc5)n4)c3)n2)cc1 ; Cc1ccc(-c2nc(-c3ccc(C)cc3)nc(-c3cccc(-c4nc(-c5ccc(F)cc5)nc(-c5c(-c6ccccc6)cccc5-c5ccccc5)n4)c3)n2)cc1]

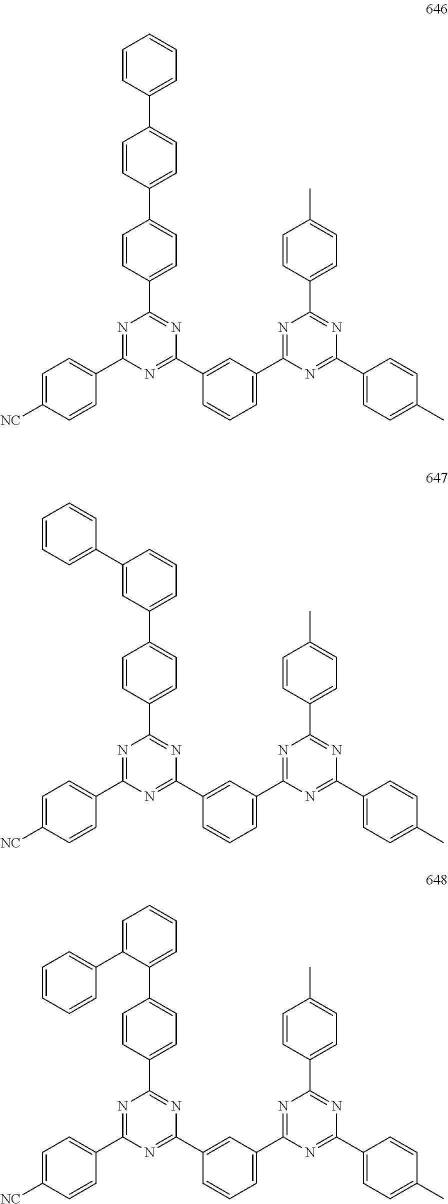 [SMILES: Cc1ccc(-c2nc(-c3ccc(C)cc3)nc(-c3cccc(-c4nc(-c5ccc(C#N)cc5)nc(-c5ccc(-c6ccc(-c7ccccc7)cc6)cc5)n4)c3)n2)cc1 ; Cc1ccc(-c2nc(-c3ccc(C)cc3)nc(-c3cccc(-c4nc(-c5ccc(C#N)cc5)nc(-c5ccc(-c6cccc(-c7ccccc7)c6)cc5)n4)c3)n2)cc1 ; Cc1ccc(-c2nc(-c3ccc(C)cc3)nc(-c3cccc(-c4nc(-c5ccc(C#N)cc5)nc(-c5ccc(-c6ccccc6-c6ccccc6)cc5)n4)c3)n2)cc1]

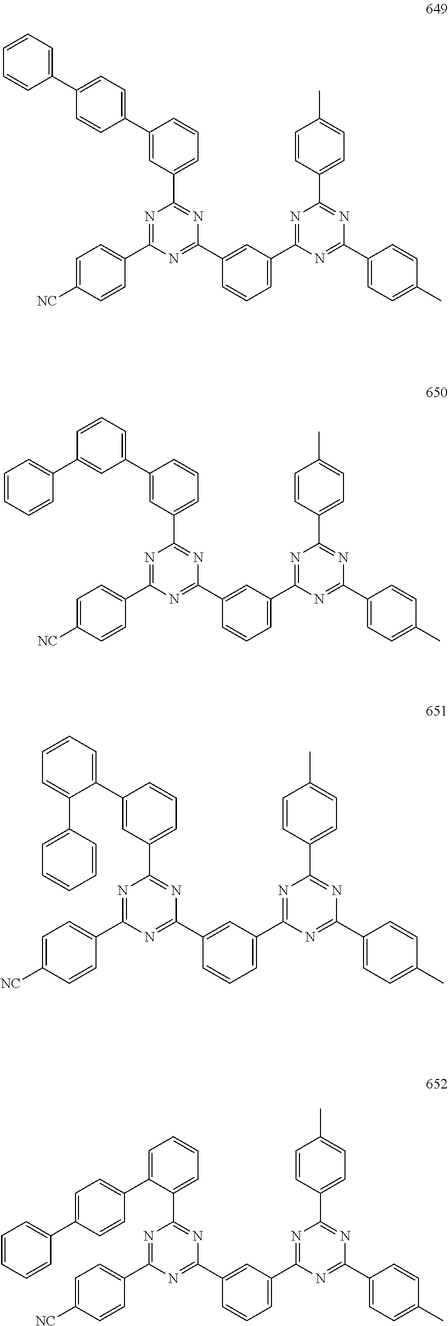 [SMILES: Cc1ccc(-c2nc(-c3ccc(C)cc3)nc(-c3cccc(-c4nc(-c5ccc(C#N)cc5)nc(-c5cccc(-c6ccc(-c7ccccc7)cc6)c5)n4)c3)n2)cc1 ; Cc1ccc(-c2nc(-c3ccc(C)cc3)nc(-c3cccc(-c4nc(-c5ccc(C#N)cc5)nc(-c5cccc(-c6cccc(-c7ccccc7)c6)c5)n4)c3)n2)cc1 ; Cc1ccc(-c2nc(-c3ccc(C)cc3)nc(-c3cccc(-c4nc(-c5ccc(C#N)cc5)nc(-c5cccc(-c6ccccc6-c6ccccc6)c5)n4)c3)n2)cc1 ; Cc1ccc(-c2nc(-c3ccc(C)cc3)nc(-c3cccc(-c4nc(-c5ccc(C#N)cc5)nc(-c5ccccc5-c5ccc(-c6ccccc6)cc5)n4)c3)n2)cc1]

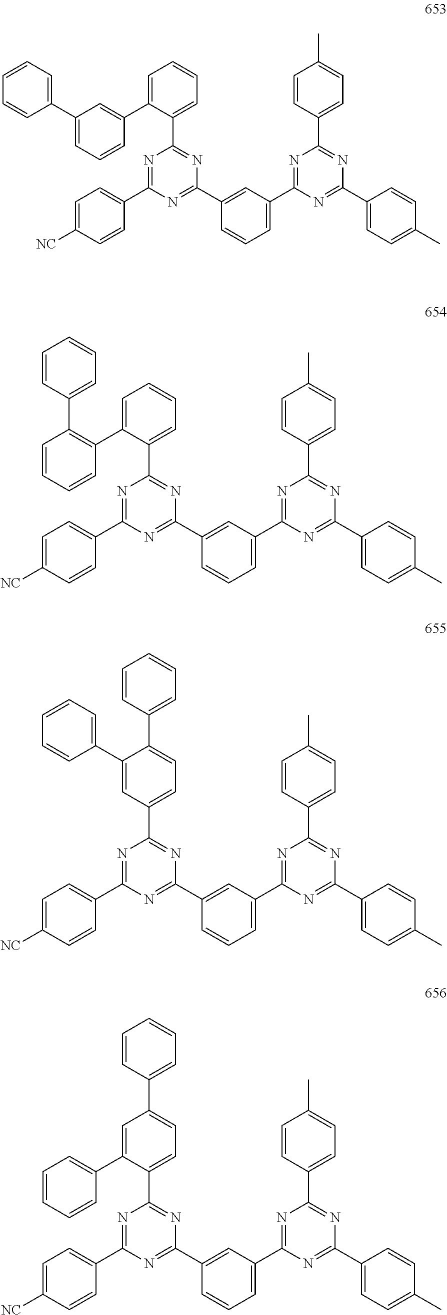 [SMILES: Cc1ccc(-c2nc(-c3ccc(C)cc3)nc(-c3cccc(-c4nc(-c5ccc(C#N)cc5)nc(-c5ccccc5-c5cccc(-c6ccccc6)c5)n4)c3)n2)cc1 ; Cc1ccc(-c2nc(-c3ccc(C)cc3)nc(-c3cccc(-c4nc(-c5ccc(C#N)cc5)nc(-c5ccccc5-c5ccccc5-c5ccccc5)n4)c3)n2)cc1 ; Cc1ccc(-c2nc(-c3ccc(C)cc3)nc(-c3cccc(-c4nc(-c5ccc(C#N)cc5)nc(-c5ccc(-c6ccccc6)c(-c6ccccc6)c5)n4)c3)n2)cc1 ; Cc1ccc(-c2nc(-c3ccc(C)cc3)nc(-c3cccc(-c4nc(-c5ccc(C#N)cc5)nc(-c5ccc(-c6ccccc6)cc5-c5ccccc5)n4)c3)n2)cc1]

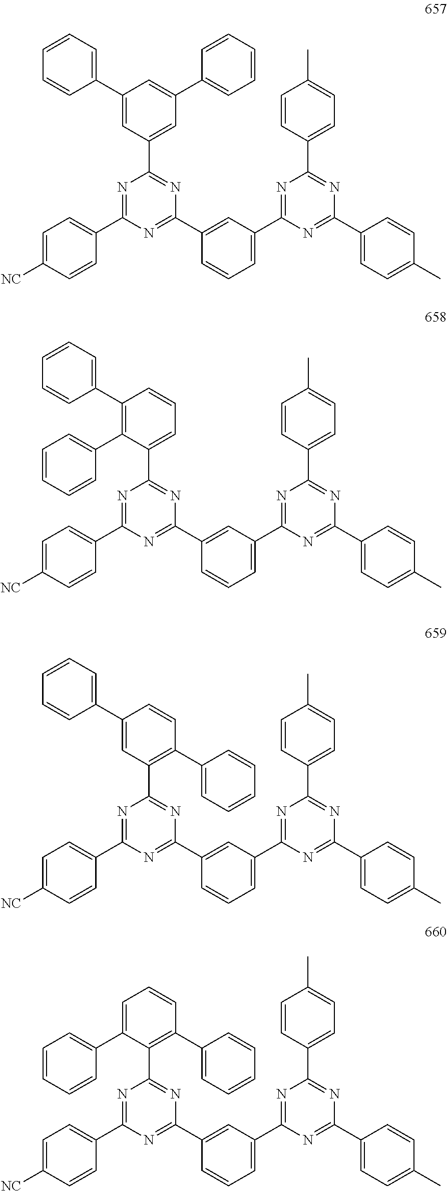 [SMILES: Cc1ccc(-c2nc(-c3ccc(C)cc3)nc(-c3cccc(-c4nc(-c5ccc(C#N)cc5)nc(-c5cc(-c6ccccc6)cc(-c6ccccc6)c5)n4)c3)n2)cc1 ; Cc1ccc(-c2nc(-c3ccc(C)cc3)nc(-c3cccc(-c4nc(-c5ccc(C#N)cc5)nc(-c5cccc(-c6ccccc6)c5-c5ccccc5)n4)c3)n2)cc1 ; Cc1ccc(-c2nc(-c3ccc(C)cc3)nc(-c3cccc(-c4nc(-c5ccc(C#N)cc5)nc(-c5cc(-c6ccccc6)ccc5-c5ccccc5)n4)c3)n2)cc1 ; Cc1ccc(-c2nc(-c3ccc(C)cc3)nc(-c3cccc(-c4nc(-c5ccc(C#N)cc5)nc(-c5c(-c6ccccc6)cccc5-c5ccccc5)n4)c3)n2)cc1]

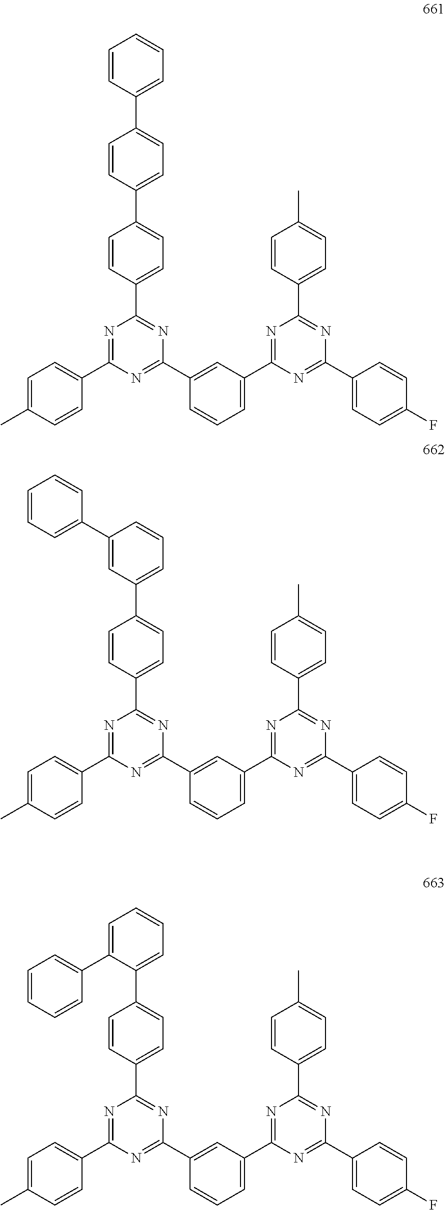 [SMILES: Cc1ccc(-c2nc(-c3ccc(F)cc3)nc(-c3cccc(-c4nc(-c5ccc(C)cc5)nc(-c5ccc(-c6ccc(-c7ccccc7)cc6)cc5)n4)c3)n2)cc1 ; Cc1ccc(-c2nc(-c3ccc(F)cc3)nc(-c3cccc(-c4nc(-c5ccc(C)cc5)nc(-c5ccc(-c6cccc(-c7ccccc7)c6)cc5)n4)c3)n2)cc1 ; Cc1ccc(-c2nc(-c3ccc(F)cc3)nc(-c3cccc(-c4nc(-c5ccc(C)cc5)nc(-c5ccc(-c6ccccc6-c6ccccc6)cc5)n4)c3)n2)cc1]

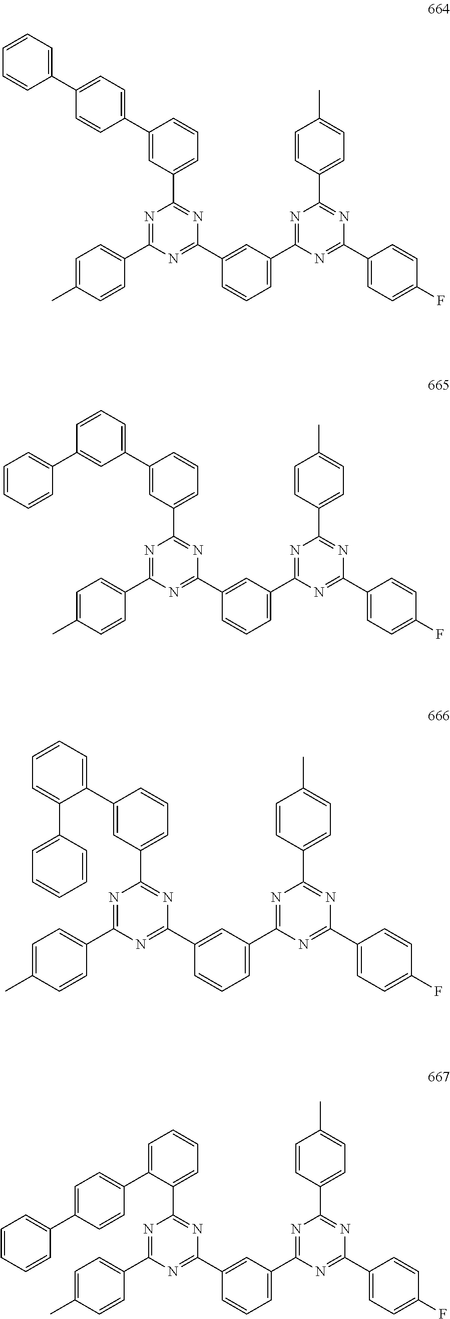 [SMILES: Cc1ccc(-c2nc(-c3ccc(F)cc3)nc(-c3cccc(-c4nc(-c5ccc(C)cc5)nc(-c5cccc(-c6ccc(-c7ccccc7)cc6)c5)n4)c3)n2)cc1 ; Cc1ccc(-c2nc(-c3ccc(F)cc3)nc(-c3cccc(-c4nc(-c5ccc(C)cc5)nc(-c5cccc(-c6cccc(-c7ccccc7)c6)c5)n4)c3)n2)cc1 ; Cc1ccc(-c2nc(-c3ccc(F)cc3)nc(-c3cccc(-c4nc(-c5ccc(C)cc5)nc(-c5cccc(-c6ccccc6-c6ccccc6)c5)n4)c3)n2)cc1 ; Cc1ccc(-c2nc(-c3ccc(F)cc3)nc(-c3cccc(-c4nc(-c5ccc(C)cc5)nc(-c5ccccc5-c5ccc(-c6ccccc6)cc5)n4)c3)n2)cc1]

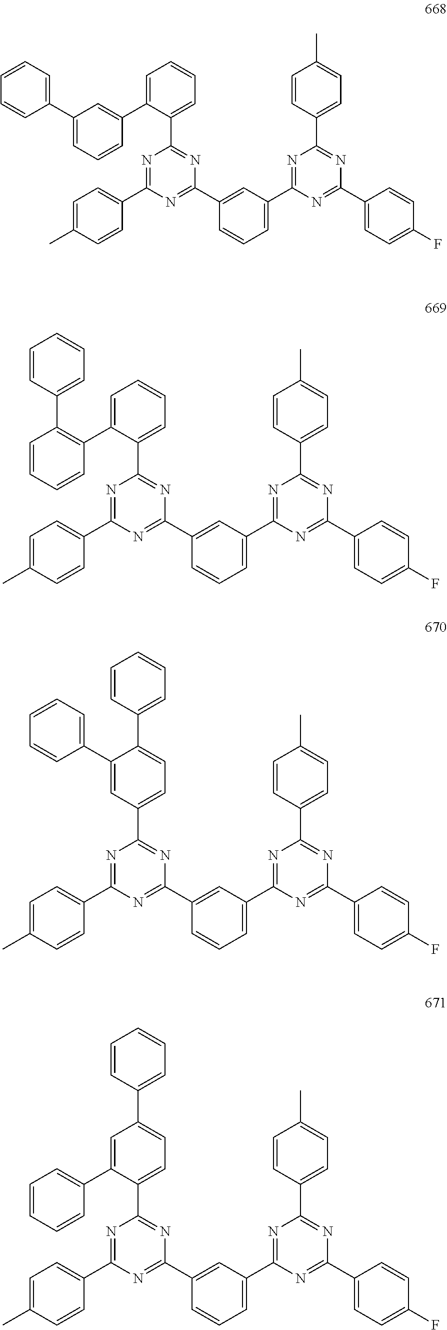 [SMILES: Cc1ccc(-c2nc(-c3ccc(F)cc3)nc(-c3cccc(-c4nc(-c5ccc(C)cc5)nc(-c5ccccc5-c5cccc(-c6ccccc6)c5)n4)c3)n2)cc1 ; Cc1ccc(-c2nc(-c3ccc(F)cc3)nc(-c3cccc(-c4nc(-c5ccc(C)cc5)nc(-c5ccccc5-c5ccccc5-c5ccccc5)n4)c3)n2)cc1 ; Cc1ccc(-c2nc(-c3ccc(F)cc3)nc(-c3cccc(-c4nc(-c5ccc(C)cc5)nc(-c5ccc(-c6ccccc6)c(-c6ccccc6)c5)n4)c3)n2)cc1 ; Cc1ccc(-c2nc(-c3ccc(F)cc3)nc(-c3cccc(-c4nc(-c5ccc(C)cc5)nc(-c5ccc(-c6ccccc6)cc5-c5ccccc5)n4)c3)n2)cc1]

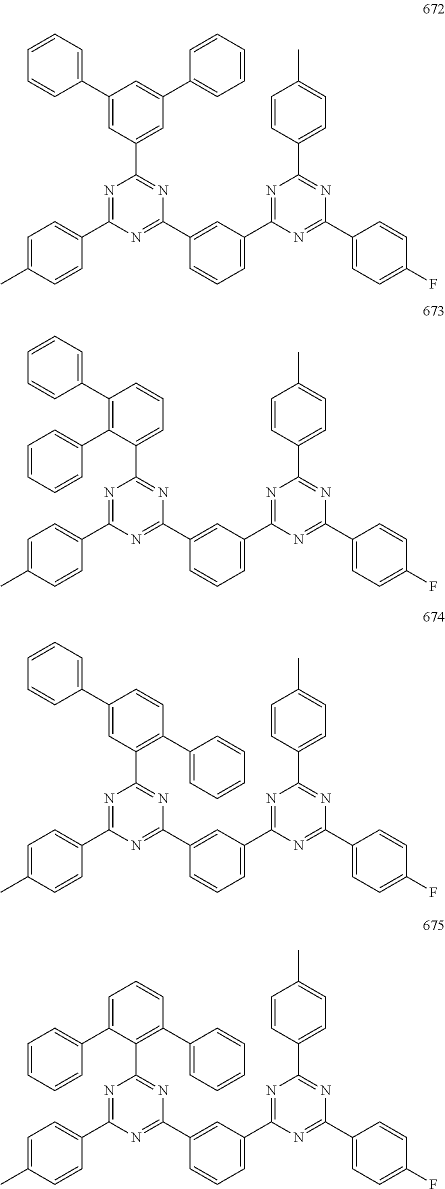 [SMILES: Cc1ccc(-c2nc(-c3ccc(F)cc3)nc(-c3cccc(-c4nc(-c5ccc(C)cc5)nc(-c5cc(-c6ccccc6)cc(-c6ccccc6)c5)n4)c3)n2)cc1 ; Cc1ccc(-c2nc(-c3ccc(F)cc3)nc(-c3cccc(-c4nc(-c5ccc(C)cc5)nc(-c5cccc(-c6ccccc6)c5-c5ccccc5)n4)c3)n2)cc1 ; Cc1ccc(-c2nc(-c3ccc(F)cc3)nc(-c3cccc(-c4nc(-c5ccc(C)cc5)nc(-c5cc(-c6ccccc6)ccc5-c5ccccc5)n4)c3)n2)cc1 ; Cc1ccc(-c2nc(-c3ccc(F)cc3)nc(-c3cccc(-c4nc(-c5ccc(C)cc5)nc(-c5c(-c6ccccc6)cccc5-c5ccccc5)n4)c3)n2)cc1]

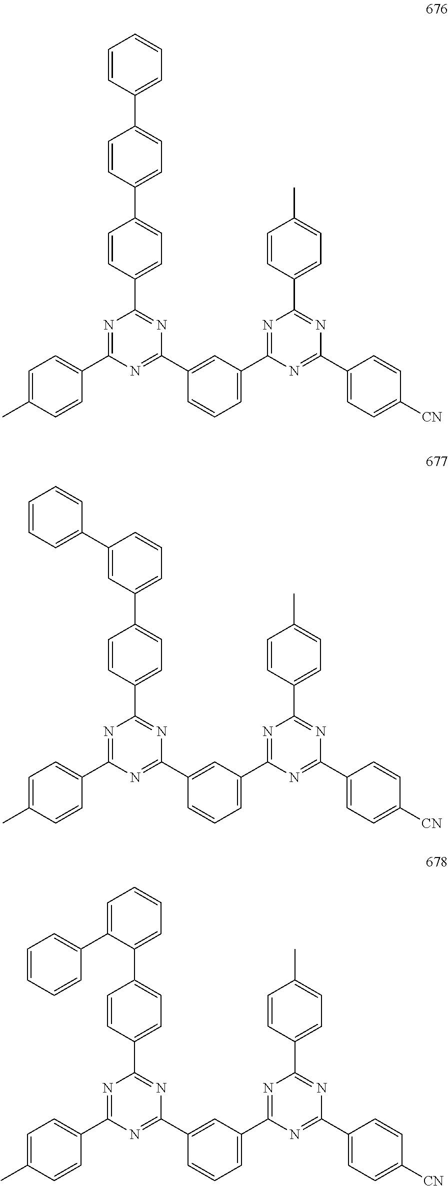 [SMILES: Cc1ccc(-c2nc(-c3ccc(C#N)cc3)nc(-c3cccc(-c4nc(-c5ccc(C)cc5)nc(-c5ccc(-c6ccc(-c7ccccc7)cc6)cc5)n4)c3)n2)cc1 ; Cc1ccc(-c2nc(-c3ccc(C#N)cc3)nc(-c3cccc(-c4nc(-c5ccc(C)cc5)nc(-c5ccc(-c6cccc(-c7ccccc7)c6)cc5)n4)c3)n2)cc1 ; Cc1ccc(-c2nc(-c3ccc(C#N)cc3)nc(-c3cccc(-c4nc(-c5ccc(C)cc5)nc(-c5ccc(-c6ccccc6-c6ccccc6)cc5)n4)c3)n2)cc1]

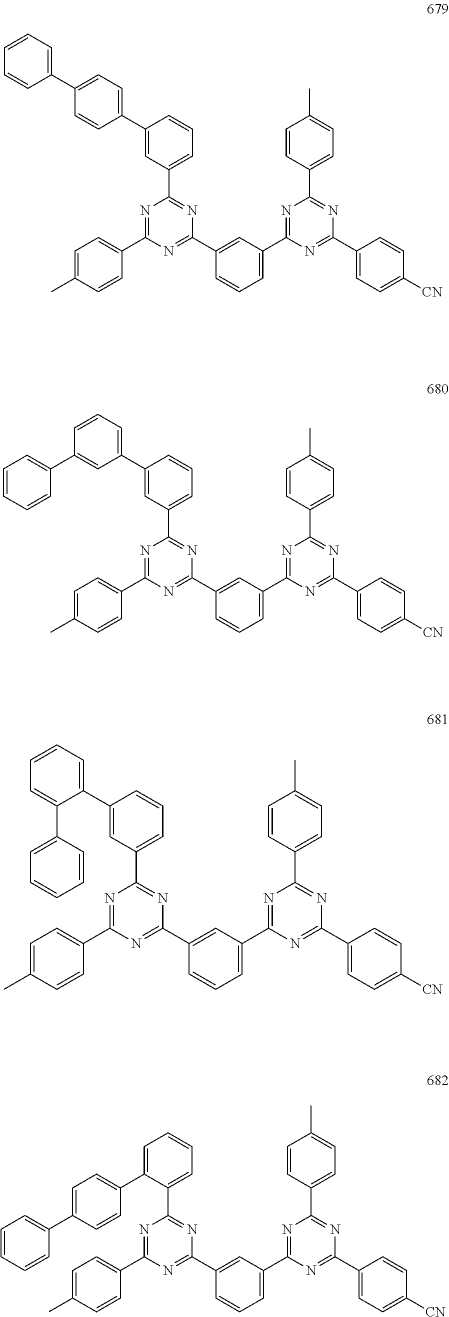 [SMILES: Cc1ccc(-c2nc(-c3ccc(C#N)cc3)nc(-c3cccc(-c4nc(-c5ccc(C)cc5)nc(-c5cccc(-c6ccc(-c7ccccc7)cc6)c5)n4)c3)n2)cc1 ; Cc1ccc(-c2nc(-c3ccc(C#N)cc3)nc(-c3cccc(-c4nc(-c5ccc(C)cc5)nc(-c5cccc(-c6cccc(-c7ccccc7)c6)c5)n4)c3)n2)cc1 ; Cc1ccc(-c2nc(-c3ccc(C#N)cc3)nc(-c3cccc(-c4nc(-c5ccc(C)cc5)nc(-c5cccc(-c6ccccc6-c6ccccc6)c5)n4)c3)n2)cc1 ; Cc1ccc(-c2nc(-c3ccc(C#N)cc3)nc(-c3cccc(-c4nc(-c5ccc(C)cc5)nc(-c5ccccc5-c5ccc(-c6ccccc6)cc5)n4)c3)n2)cc1]

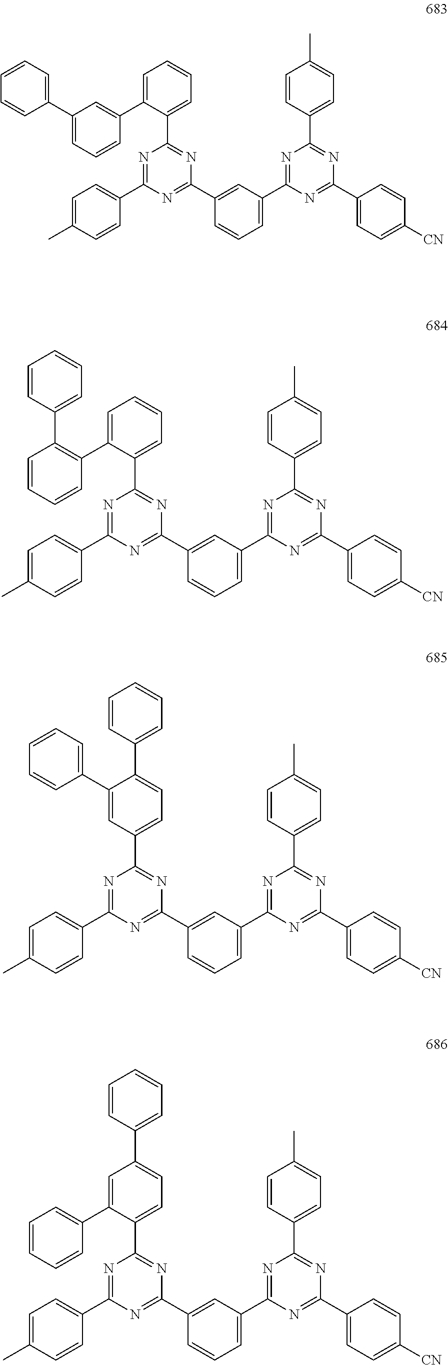 [SMILES: Cc1ccc(-c2nc(-c3ccc(C#N)cc3)nc(-c3cccc(-c4nc(-c5ccc(C)cc5)nc(-c5ccccc5-c5cccc(-c6ccccc6)c5)n4)c3)n2)cc1 ; Cc1ccc(-c2nc(-c3ccc(C#N)cc3)nc(-c3cccc(-c4nc(-c5ccc(C)cc5)nc(-c5ccccc5-c5ccccc5-c5ccccc5)n4)c3)n2)cc1 ; Cc1ccc(-c2nc(-c3ccc(C#N)cc3)nc(-c3cccc(-c4nc(-c5ccc(C)cc5)nc(-c5ccc(-c6ccccc6)c(-c6ccccc6)c5)n4)c3)n2)cc1 ; Cc1ccc(-c2nc(-c3ccc(C#N)cc3)nc(-c3cccc(-c4nc(-c5ccc(C)cc5)nc(-c5ccc(-c6ccccc6)cc5-c5ccccc5)n4)c3)n2)cc1]

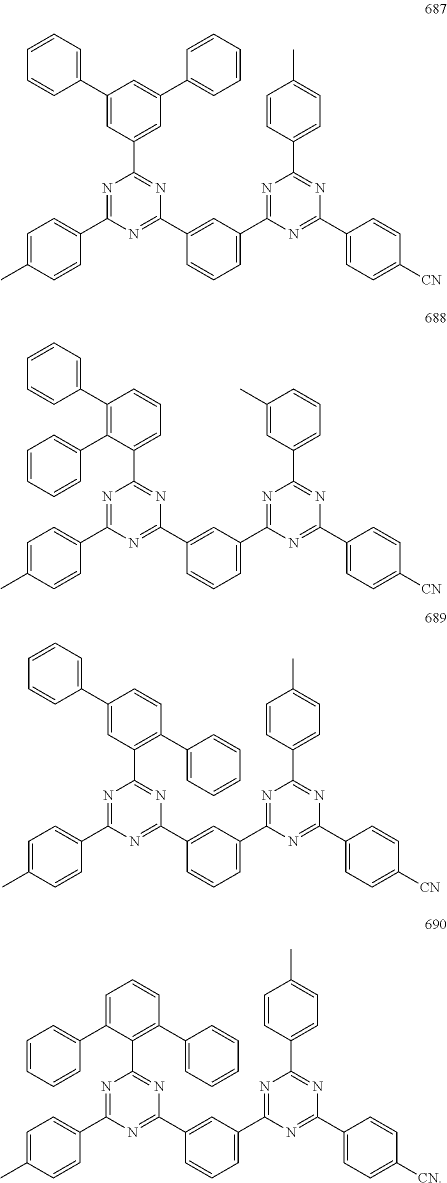 [SMILES: Cc1ccc(-c2nc(-c3ccc(C#N)cc3)nc(-c3cccc(-c4nc(-c5ccc(C)cc5)nc(-c5cc(-c6ccccc6)cc(-c6ccccc6)c5)n4)c3)n2)cc1 ; Cc1ccc(-c2nc(-c3cccc(-c4nc(-c5ccc(C#N)cc5)nc(-c5cccc(C)c5)n4)c3)nc(-c3cccc(-c4ccccc4)c3-c3ccccc3)n2)cc1 ; Cc1ccc(-c2nc(-c3ccc(C#N)cc3)nc(-c3cccc(-c4nc(-c5ccc(C)cc5)nc(-c5cc(-c6ccccc6)ccc5-c5ccccc5)n4)c3)n2)cc1 ; Cc1ccc(-c2nc(-c3ccc(C#N)cc3)nc(-c3cccc(-c4nc(-c5ccc(C)cc5)nc(-c5c(-c6ccccc6)cccc5-c5ccccc5)n4)c3)n2)cc1]

A light-emitting device according to another aspect of embodiments includes:
a first electrode;
a second electrode facing the first electrode; and
an interlayer between the first electrode and the second electrode and including an emission layer, wherein:
the interlayer may include the compound represented by Formula 1.

According to an embodiment, the first electrode may be an anode, the second electrode may be a cathode, and the interlayer may further include a hole transport region between the first electrode and the emission layer and including a hole injection layer, a hole transport layer, an electron blocking layer, an emission auxiliary layer, or any combination thereof.

According to an embodiment, the first electrode may be an anode, the second electrode may be a cathode, and the interlayer may further include an electron transport region between the second electrode and the emission layer and including a hole blocking layer, an electron transport layer, an electron injection layer, or any combination thereof.

According to an embodiment, the electron transport region may include the compound represented by Formula 1. For example, the hole-blocking layer may include the compound represented by Formula 1. For example, the electron transport layer may include the compound represented by Formula 1. For example, the electron injection layer may include the compound represented by Formula 1.

According to an embodiment, the emission layer may include a first host, a second host, a first dopant, and a second dopant, wherein the first dopant may be a compound including a ligand including a metal and an imidazole moiety, and the second dopant may be a compound including boron.

According to an embodiment, the first host may be a hole-transporting host.

According to an embodiment, the second host may be an electron-transporting host.

The hole-transporting host may be a compound having strong hole properties. The expression “a compound having strong hole properties” refers to a compound that is easy to accept holes (e.g., that easily accepts and/or transports holes), and such properties may be obtained by including a hole-receiving moiety (also, referred to as an HT moiety).

Such a hole-receiving moiety may include, for example, a π electron-rich heteroaromatic compound (e.g., a carbazole derivative and/or an indole derivative), and/or an aromatic amine compound.

The electron-transporting host may be a compound having strong electron properties. The expression “a compound having strong electron properties” refers to a compound that is easy to accept electrons (e.g., that easily accepts and/or transports electrons), and such properties may be obtained by including an electron-receiving moiety (also, referred to as an ET moiety).

Such an electron-receiving moiety may include, for example, a π electron-deficient heteroaromatic compound. For example, the electron-receiving moiety may include a nitrogen-containing heteroaromatic compound.

In embodiments, for a compound that includes only HT moieties or only ET moieties, the nature of the compound as being HT or ET is clear.

A compound may include both HT moieties and ET moieties. In such embodiments, a simple comparison of the total number of HT moieties and the total number of ET moieties present in the compound may serve as a criterion for predicting or determining whether the compound is HT or ET, but cannot be an absolute criterion. One reason for this is the fact that one HT moiety and one ET moiety may not have exactly the same ability to attract holes and electrons, respectively.

Therefore, a relatively certain method of determining whether a compound of a given structure is HT or ET may be to implement the compound directly in a device.

According to an embodiment, the first host and the second host may have a weight ratio of 9:1 to 1:9. For example, the first host and the second host may have a weight ratio of 6:4 to 4:6. In embodiments where the weight ratio of the first host and the second host is within this range, the charge injection balance may be suitable or appropriate.

According to an embodiment, the metal of the first dopant may be a transition metal.

For example, the first dopant may include a compound represented by Formula 401:

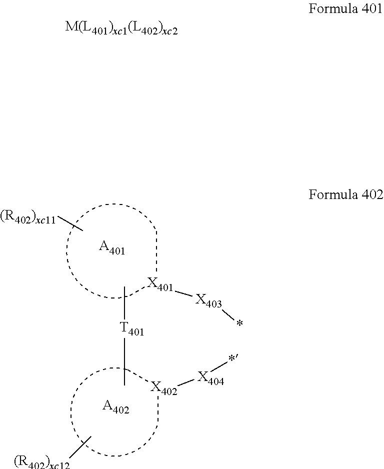

wherein, in Formulae 401 and 402,
M may be titanium (Ti), cobalt (Co), copper (Cu), zinc (Zn), zirconium (Zr), ruthenium (Ru), rhodium (Rh), palladium (Pd), rhenium (Re), platinum (Pt), gold (Au), osmium (Os), iridium (Ir), or rhenium (Re),
L₄₀₁ may be a ligand represented by Formula 402, and xc1 is 1, 2, or 3, wherein, when xc1 is 2 or more, two or more of L₄₀₁ may be identical to or different from each other,
L₄₀₂ may be an organic ligand, and xc2 may be 0, 1, 2, 3, or 4, wherein, when xc2 is 2 or more, two or more of L₄₀₂ may be identical to or different from each other,
X₄₀₁ and X₄₀₂ may each independently be nitrogen or carbon,
ring A₄₀₁ and ring A₄₀₂ may each independently be a C₃-C₆₀ carbocyclic group or a C₁-C₆₀ heterocyclic group,
T₄₀₁ may be a single bond (e.g., a covalent single bond), —O—, —S—, —C(═O)—, —N(Q₄₁₁)-, —C(Q₄₁₁)(Q₄₁₂)-, —C(Q₄₁₁)=C(Q₄₁₂)-, —C(Q₄₁₁)=, or ═C═,
X₄₀₃ and X₄₀₄ may each independently be a chemical bond (e.g., a covalent chemical bond or a coordinate covalent bond, which may also be referred to as a dative bond), O, S, N(Q₄₁₃), B(Q₄₁₃), P(Q₄₁₃), C(Q₄₁₃)(Q₄₁₄), or Si(Q₄₁₃)(Q₄₁₄),
R₄₀₁ and R₄₀₂ may each independently be hydrogen, deuterium, —F, —Cl, —Br, —I, a hydroxyl group, a cyano group, a nitro group, a C₁-C₂₀ alkyl group that is unsubstituted or substituted with at least one R₁₀ₐ, a C₁-C₂₀ alkoxy group that is unsubstituted or substituted with at least one R₁₀ₐ, a C₃-C₆₀ carbocyclic group that is unsubstituted or substituted with at least one R₁₀ₐ, a C₁-C₆₀ heterocyclic group that is unsubstituted or substituted with at least one R₁₀ₐ, —Si(Q₄₀₁)(Q₄₀₂)(Q₄₀₃), —N(Q₄₀₁)(Q₄₀₂), —B(Q₄₀₁)(Q₄₀₂), —C(═O)(Q₄₀₁), —S(═O)₂(Q₄₀₁), or —P(═O)(Q₄₀₁)(Q₄₀₂),
optionally, R₄₀₁ and R₄₀₂ may be linked to each other to form a ring,
R₁₀ₐ may be:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁₁ to Q₁₃, Q₂₁ to Q₂₃, Q₃₁ to Q₃₃, Q₄₁₁ to Q₄₁₄ and Q₄₀₁ to Q₄₀₃ may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof,
xc11 and xc12 may each independently be an integer from 0 to 10,
* and *′ in Formula 402 each indicate a binding site to M in Formula 401, and
one of ring A₄₀₁ or ring A₄₀₂ may include an imidazole moiety.

In one or more embodiments, when xc1 in Formula 401 is 2 or more, two of ring A₄₀₁ among two or more of L₄₀₁ may be optionally linked to each other via T₄₀₂, which is a linking group, or two of ring A₄₀₂ among two or more of L₄₀₁ may be optionally linked to each other via T₄₀₃, which is a linking group. T₄₀₂ and T₄₀₃ are each as described in connection with T₄₀₁.

According to an embodiment, the second dopant may include a compound represented by Formula 2:

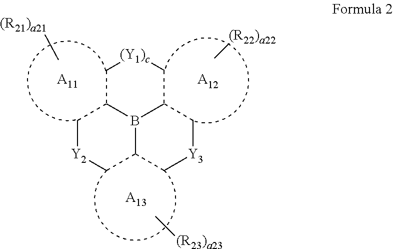

wherein, in Formula 2, Y₁ to Y₃ may each independently be S, N(R₂₄), B(R₂₄), C(R₂₄)(R₂₅), or Si(R₂₄)(R₂₅),
c may be 0 or 1,
A₁₁ to A₁₃ may each independently be selected from a C₅-C₃₀ carbocyclic group and a C₁-C₃₀ heterocyclic group,
R₂₁ and R₂₅ may each independently be hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₁₀ cycloalkyl group unsubstituted or substituted with at least one R₁₀ₐ, a C₁-C₁₀ heterocycloalkyl group unsubstituted or substituted with at least one R₁₀ₐ, a C₃-C₁₀ cycloalkenyl group unsubstituted or substituted with at least one R₁₀ₐ, a C₁-C₁₀ heterocycloalkenyl group unsubstituted or substituted with at least one R₁₀ₐ, a C₆-C₆₀ aryl group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₁-C₆₀ heteroaryl group unsubstituted or substituted with at least one R₁₀ₐ, a monovalent non-aromatic condensed polycyclic group unsubstituted or substituted with at least one R₁₀ₐ, a monovalent non-aromatic condensed heteropolycyclic group unsubstituted or substituted with at least one R₁₀ₐ, —Si(Q₁)(Q₂)(Q₃), —N(Q₁)(Q₂), —B(Q₁)(Q₂), —P(Q₁)(Q₂), —C(═O)(Q₁), —S(═O)₂(Q₁), and —P(═O)(Q₁)(Q₂),
R₂₁ to R₂₅ may optionally be linked to each other to form a substituted or unsubstituted C₅-C₃₀ carbocyclic group and a substituted or unsubstituted C₁-C₃₀ heterocyclic group,
a21 to a23 may each independently be an integer from 0 to 10,
R₁₀ₐ may be:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof.

According to an embodiment, the emission layer may be a fluorescent emission layer.

According to an embodiment, the emission layer may be a blue emission layer.

Another aspect of embodiments of the disclosure provides an electronic apparatus including the light-emitting device. The electronic apparatus may further include a thin-film transistor. For example, the electronic apparatus may further include a thin-film transistor including a source electrode and a drain electrode, wherein the first electrode of the light-emitting device may be electrically connected to the source electrode or the drain electrode.

According to an embodiment, the electronic apparatus may further include a color filter, a color conversion layer, a touch screen layer, a polarizing layer, or any combination thereof. More details on the electronic apparatus may be the same as the descriptions provided elsewhere herein.

The term “interlayer” as used herein refers to a single layer and/or all layers between a first electrode and a second electrode of a light-emitting device.

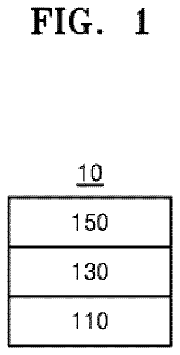

### Description of FIG. 1

FIG. 1 is a schematic cross-sectional view of a light-emitting device 10 according to an embodiment. The light-emitting device 10 includes a first electrode 110, an interlayer 130, and a second electrode 150.

Hereinafter, a structure of the light-emitting device 10 according to an embodiment and a method of manufacturing the light-emitting device 10 are described with reference to FIG. 1.

### First Electrode 110

In FIG. 1, a substrate may be under the first electrode 110 and/or on the second electrode 150. As the substrate, a glass substrate and/or a plastic substrate may be used. According to an embodiment, the substrate may be a flexible substrate and may include plastics having excellent heat resistance and durability, such as polyimide, polyethylene terephthalate (PET), polycarbonate, polyethylene naphthalate, polyarylate (PAR), polyetherimide, or any combination thereof.

The first electrode 110 may be formed by, for example, depositing and/or sputtering a material for forming the first electrode 110 on the substrate. When the first electrode 110 is an anode, a material for forming the first electrode 110 may be a high-work function material that facilitates injection of holes.

The first electrode 110 may be a reflective electrode, a transflective electrode, or a transmissive electrode. When the first electrode 110 is a transmissive electrode, a material for forming the first electrode 110 may include indium tin oxide (ITO), indium zinc oxide (IZO), tin oxide (SnO₂), zinc oxide (ZnO), or any combination thereof. According to an embodiment, when the first electrode 110 is a transflective electrode or a reflective electrode, a material included in the first electrode 110 may include magnesium (Mg), silver (Ag), aluminum (AI), aluminum-lithium (Al—Li), calcium (Ca), magnesium-indium (Mg—In), magnesium-silver (Mg—Ag), or any combination thereof.

The first electrode 110 may have a single-layer structure consisting of a single layer or a multilayer structure including a plurality of layers. According to an embodiment, the first electrode 110 may have a three-layer structure of ITO/Ag/ITO.

### Interlayer 130

The interlayer 130 is above the first electrode 110. The interlayer 130 includes the emission layer.

The interlayer 130 may further include a hole transport region between the first electrode 110 and the emission layer, and an electron transport region between the emission layer and the second electrode 150.

The interlayer 130 may further include, in addition to various suitable organic materials, a metal-containing compound such as an organometallic compound, an inorganic material such as quantum dots, and/or the like.

The interlayer 130 may include i) two or more emitting units sequentially stacked between the first electrode 110 and the second electrode 150, and ii) a charge generation layer between adjacent two of the two or more emitting units. When the interlayer 130 includes the emission layer and the charge generation layer as described above, the light-emitting device 10 may be a tandem light-emitting device.

### Hole Transport Region in Interlayer 130

The hole transport region may have: i) a single-layer structure consisting of a single layer consisting of a single material, ii) a single-layer structure consisting of a single layer consisting of a plurality of materials that are different from each other, or iii) a multilayer structure including a plurality of layers including a plurality of materials that are different from each other.

The hole transport region may include a hole injection layer, a hole transport layer, an emission auxiliary layer, an electron blocking layer, or any combination thereof.

For example, the hole transport region may have a multi-layer structure including a hole injection layer/hole transport layer structure, a hole injection layer/hole transport layer/emission auxiliary layer structure, a hole injection layer/emission auxiliary layer structure, a hole transport layer/emission auxiliary layer structure, or a hole injection layer/hole transport layer/electron-blocking layer structure, wherein layers in each structure are sequentially stacked from the first electrode 110.

For example, the hole transport region may have a multi-layered structure of hole transport layer/emission auxiliary layer, or hole transport layer/electron blocking layer, which are sequentially stacked in this stated order from the first electrode 110.

The hole transport region may include a compound represented by Formula 201, a compound represented by Formula 202, or any combination thereof:

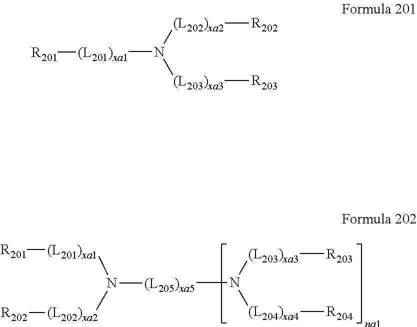

wherein, in Formulae 201 and 202,
L₂₀₁ to L₂₀₄ may each independently be a C₃-C₆₀ carbocyclic group that is unsubstituted or substituted with at least one R₁₀ₐ or a C₁-C₆₀ heterocyclic group that is unsubstituted or substituted with at least one R₁₀ₐ,
L₂₀₅ may be *—O—*′, *—S—*′, *—N(Q₂₀₁)-*′, a C₁-C₂₀ alkylene group unsubstituted or substituted with at least one R₁₀ₐ, a C₂-C₂₀ alkenylene group unsubstituted or substituted with at least one R₁₀ₐ, a C₃-C₆₀ carbocyclic group unsubstituted or substituted with at least one R₁₀ₐ, or a C₁-C₆₀ heterocyclic group unsubstituted or substituted with at least one R₁₀ₐ,
xa1 to xa4 may each independently be an integer from 0 to 5,
xa5 may be an integer from 1 to 10,
R₂₀₁ to R₂₀₄ and Q₂₀₁ may each independently be a C₃-C₆₀ carbocyclic group that is unsubstituted or substituted with at least one R₁₀ₐ or a C₁-C₆₀ heterocyclic group that is unsubstituted or substituted with at least one R₁₀ₐ,
R₂₀₁ and R₂₀₂ may optionally be linked to each other via a single bond (e.g., a covalent single bond), a C₁-C₅ alkylene group that is unsubstituted or substituted with at least one R₁₀ₐ, or a C₂-C₅ alkenylene group that is unsubstituted or substituted with at least one R₁₀ₐ to form a C₈-C₆₀ polycyclic group (for example, a carbazole group) that is unsubstituted or substituted with at least one R₁₀ₐ (for example, Compound HT16),
R₂₀₃ and R₂₀₄ may optionally be linked to each other via a single bond (e.g., a covalent single bond), a C₁-C₅ alkylene group that is unsubstituted or substituted with at least one R₁₀ₐ, or a C₂-C₅ alkenylene group that is unsubstituted or substituted with at least one R₁₀ₐ to form a C₈-C₆₀ polycyclic group that is unsubstituted or substituted with at least one R₁₀ₐ, and
na1 may be an integer from 1 to 4.

According to an embodiment, each of Formulae 201 and 202 may include at least one selected from among groups represented by Formulae CY201 to CY217:

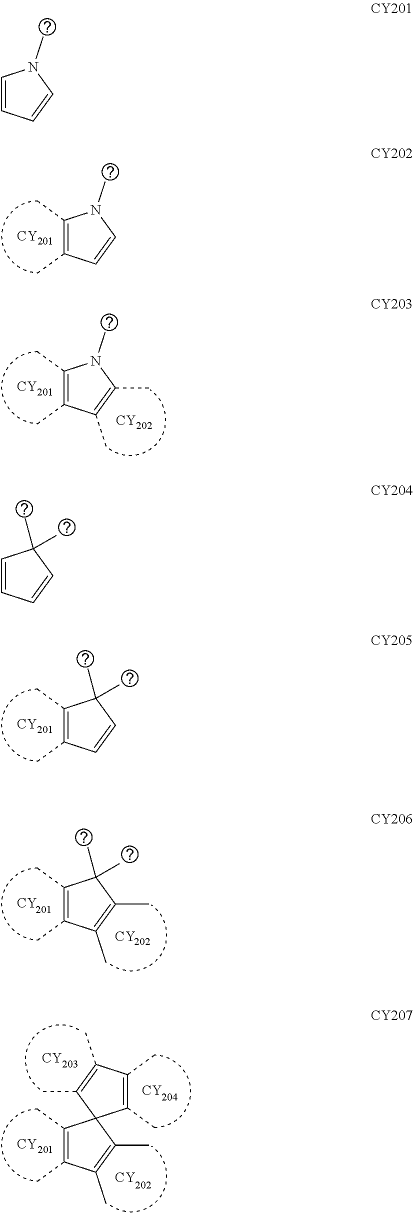

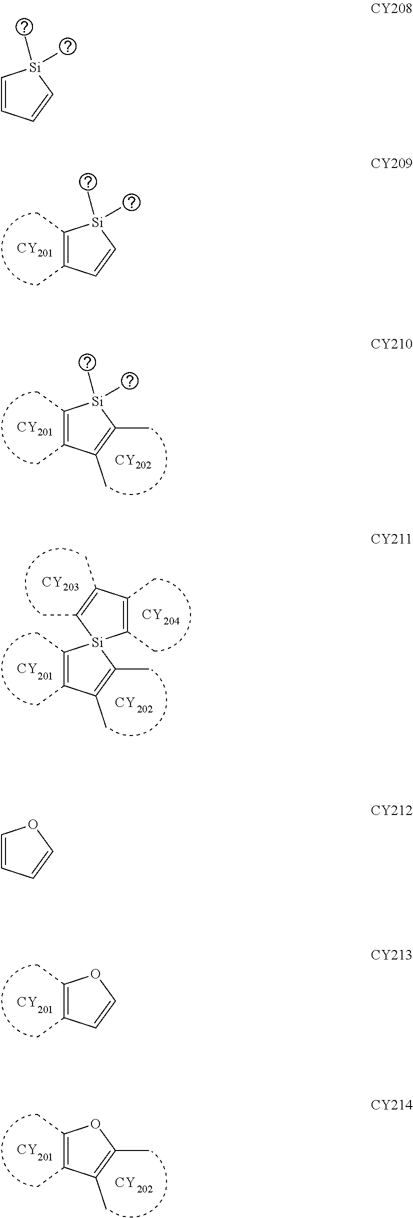

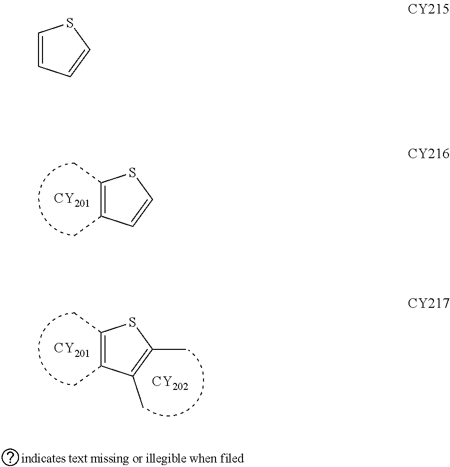

wherein, in Formulae CY201 to CY217, R_{10b}, and R_{10c} are each as described in connection with R₁₀ₐ, ring CY₂₀₁ to ring CY₂₀₄ may each independently be a C₃-C₂₀ carbocyclic group or a C₁-C₂₀ heterocyclic group, and at least one hydrogen in Formulae CY201 to CY217 may be unsubstituted or substituted with R₁₀ₐ.

According to an embodiment, ring CY₂₀₁ to ring CY₂₀₄ in Formulae CY201 to CY217 may each independently be a benzene group, a naphthalene group, a phenanthrene group, or an anthracene group.

According to an embodiment, each of Formulae 201 and 202 may include at least one selected from among groups represented by Formulae CY201 to CY203.

According to an embodiment, Formula 201 may include at least one selected from among groups represented by Formulae CY201 to CY203 and at least one selected from among groups represented by Formulae CY204 to CY217.

According to an embodiment, in Formula 201, xa1 may be 1, R₂₀₁ may be a group represented by one selected from among Formulae CY201 to CY203, xa2 may be 0, and R₂₀₂ may be a group represented by one selected from among Formulae CY204 to CY207.

According to an embodiment, each of Formulae 201 and 202 may not include groups represented by Formulae CY201 to CY203.

According to an embodiment, each of Formulae 201 and 202 may not include groups represented by Formulae CY201 to CY203 and may include at least one selected from among groups represented by Formulae CY204 to CY217.

According to an embodiment, each of Formulae 201 and 202 may not include groups represented by Formulae CY201 to CY217.

According to an embodiment, the hole transport region may include one selected from among Compounds HT1 to HT46, m-MTDATA, TDATA, 2-TNATA, NPB(NPD), β-NPB, TPD, Spiro-TPD, Spiro-NPB, methylated NPB, TAPC, HMTPD, 4,4′,4″-tris(N-carbazolyl)triphenylamine (TCTA), polyaniline/dodecylbenzenesulfonic acid (PANI/DBSA), poly(3,4-ethylenedioxythiophene)/poly(4-styrenesulfonate) (PEDOT/PSS), polyaniline/camphor sulfonic acid (PANI/CSA), polyaniline/poly(4-styrenesulfonate) (PANI/PSS), or any combination thereof:

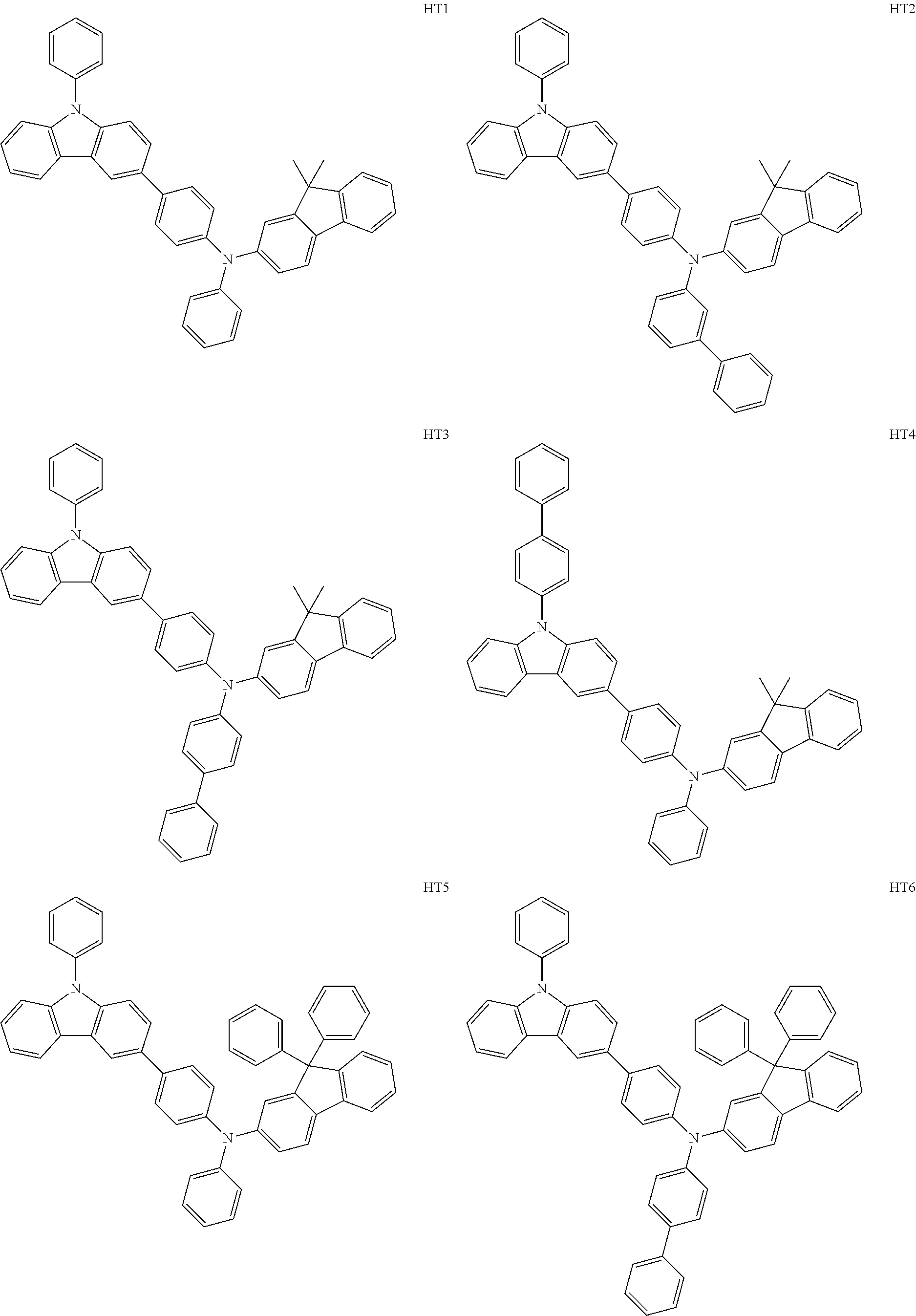

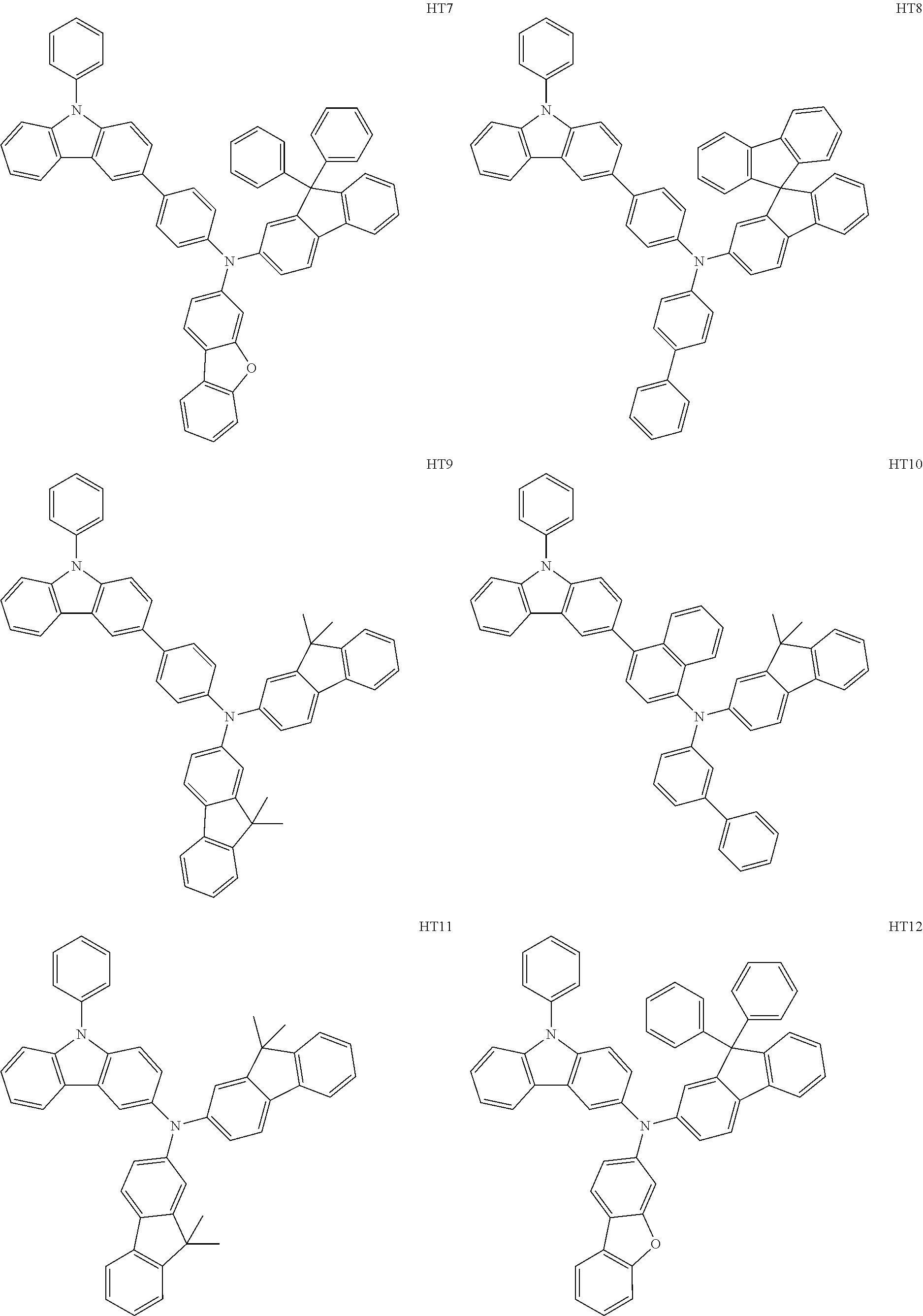

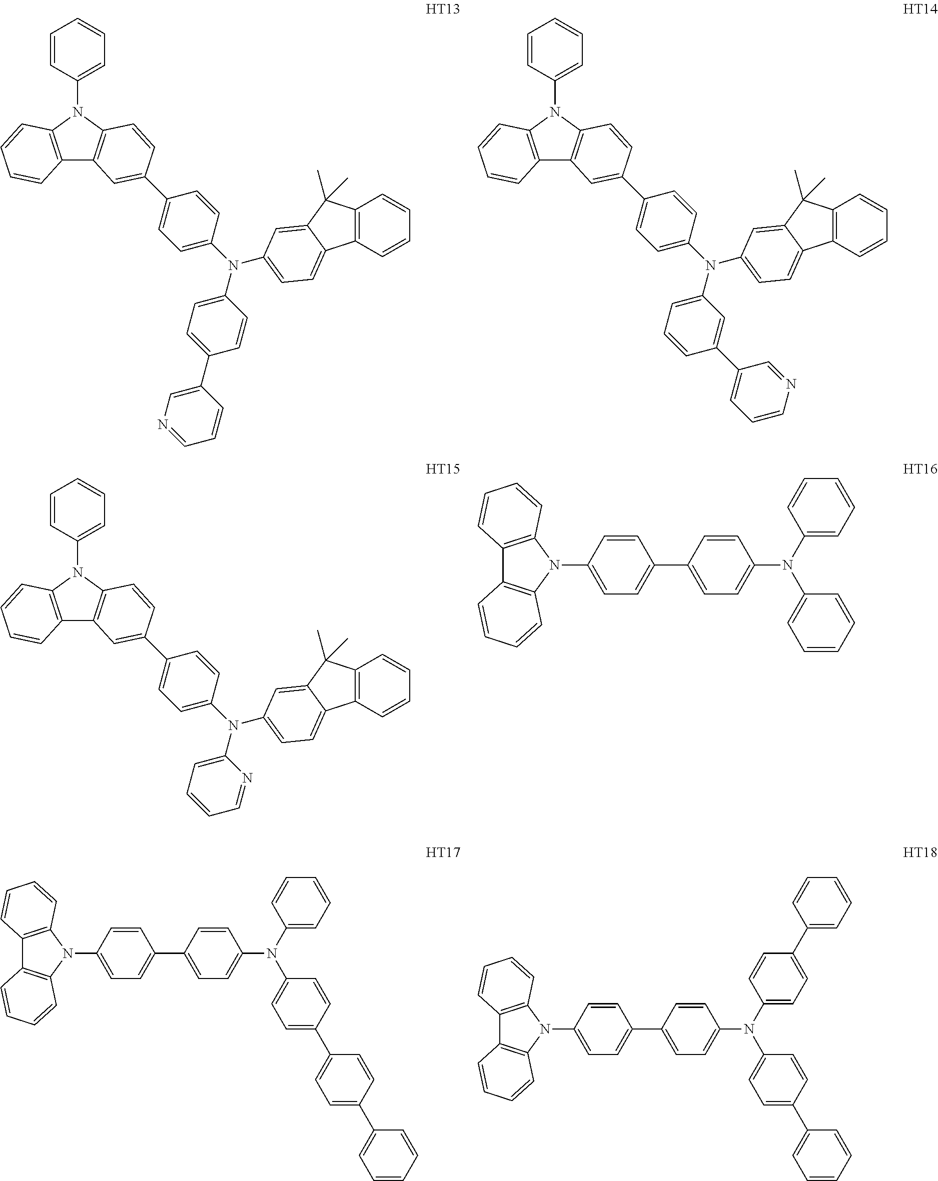

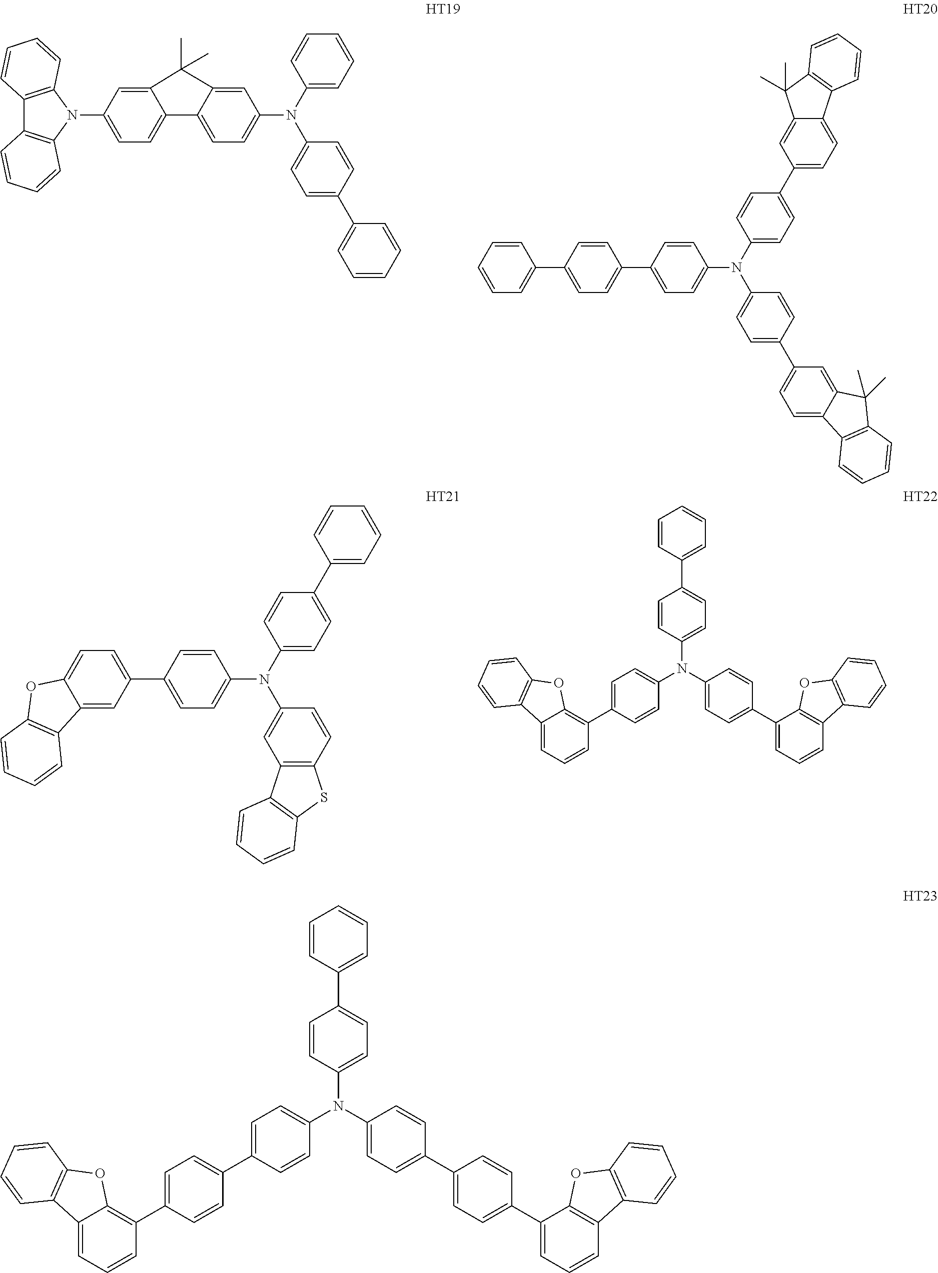 [SMILES: CC1(C)c2cc(N(c3ccccc3)c3ccc(-c4ccccc4)cc3)ccc2-c2ccc(-n3c4ccccc4c4ccccc43)cc21 ; CC1(C)c2ccccc2-c2ccc(-c3ccc(N(c4ccc(-c5ccc(-c6ccccc6)cc5)cc4)c4ccc(-c5ccc6c(c5)C(C)(C)c5ccccc5-6)cc4)cc3)cc21 ; c1ccc(-c2ccc(N(c3ccc(-c4ccc5oc6ccccc6c5c4)cc3)c3ccc4sc5ccccc5c4c3)cc2)cc1 ; c1ccc(-c2ccc(N(c3ccc(-c4cccc5c4oc4ccccc45)cc3)c3ccc(-c4cccc5c4oc4ccccc45)cc3)cc2)cc1 ; c1ccc(-c2ccc(N(c3ccc(-c4ccc(-c5cccc6c5oc5ccccc56)cc4)cc3)c3ccc(-c4ccc(-c5cccc6c5oc5ccccc56)cc4)cc3)cc2)cc1]

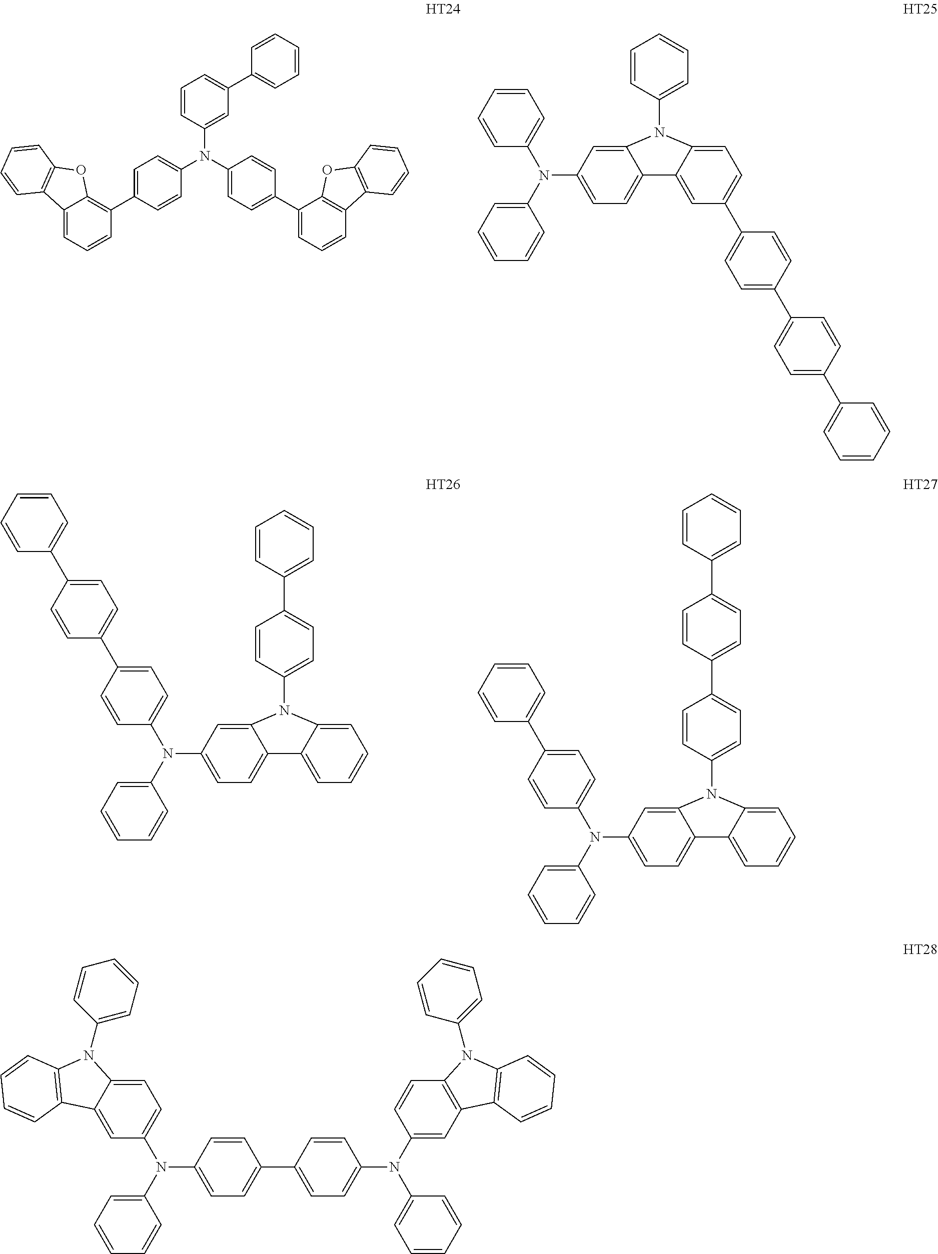 [SMILES: c1ccc(-c2cccc(N(c3ccc(-c4cccc5c4oc4ccccc45)cc3)c3ccc(-c4cccc5c4oc4ccccc45)cc3)c2)cc1 ; c1ccc(-c2ccc(-c3ccc(-c4ccc5c(c4)c4ccc(N(c6ccccc6)c6ccccc6)cc4n5-c4ccccc4)cc3)cc2)cc1 ; c1ccc(-c2ccc(-c3ccc(N(c4ccccc4)c4ccc5c6ccccc6n(-c6ccc(-c7ccccc7)cc6)c5c4)cc3)cc2)cc1 ; c1ccc(-c2ccc(-c3ccc(-n4c5ccccc5c5ccc(N(c6ccccc6)c6ccc(-c7ccccc7)cc6)cc54)cc3)cc2)cc1 ; c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1]

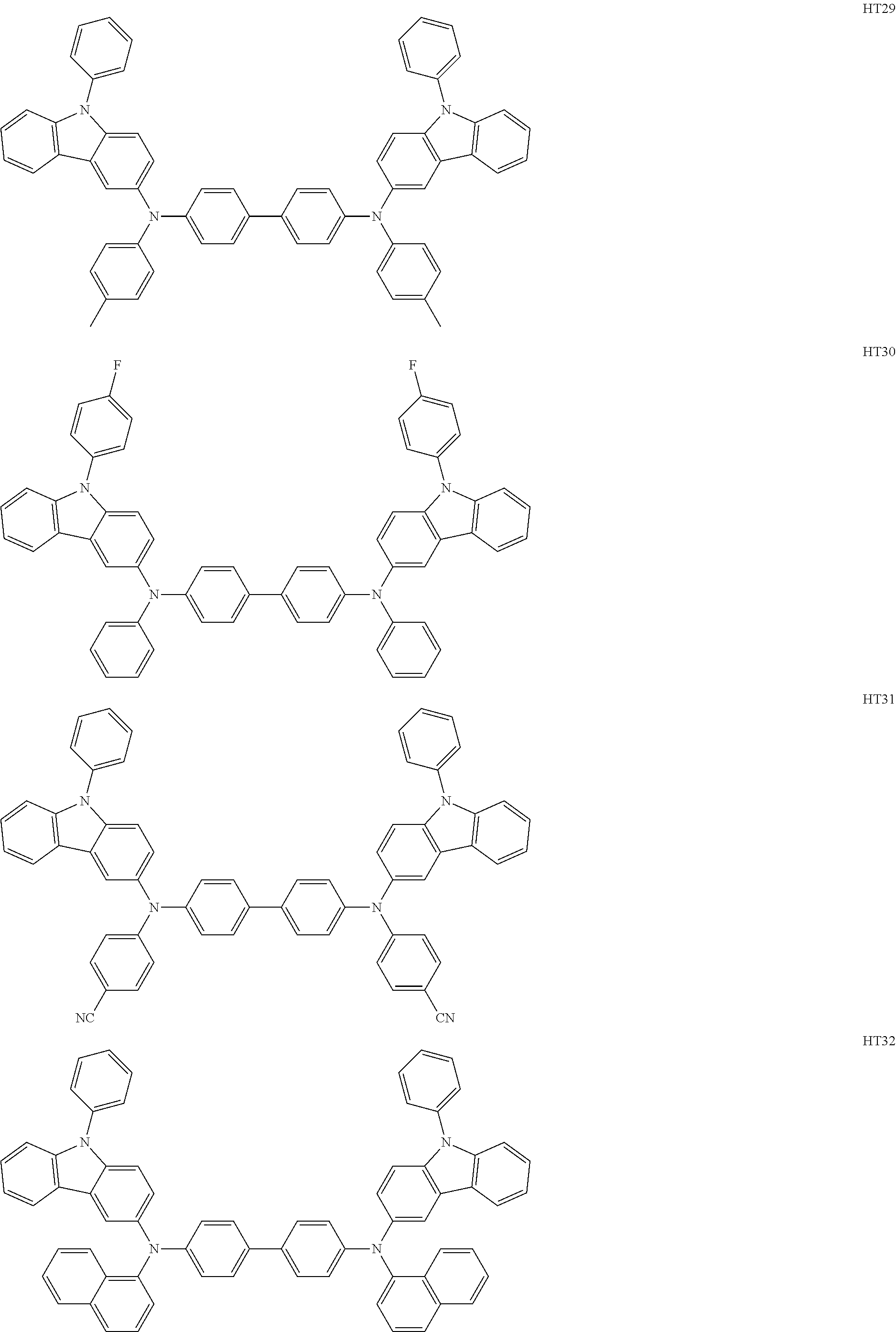

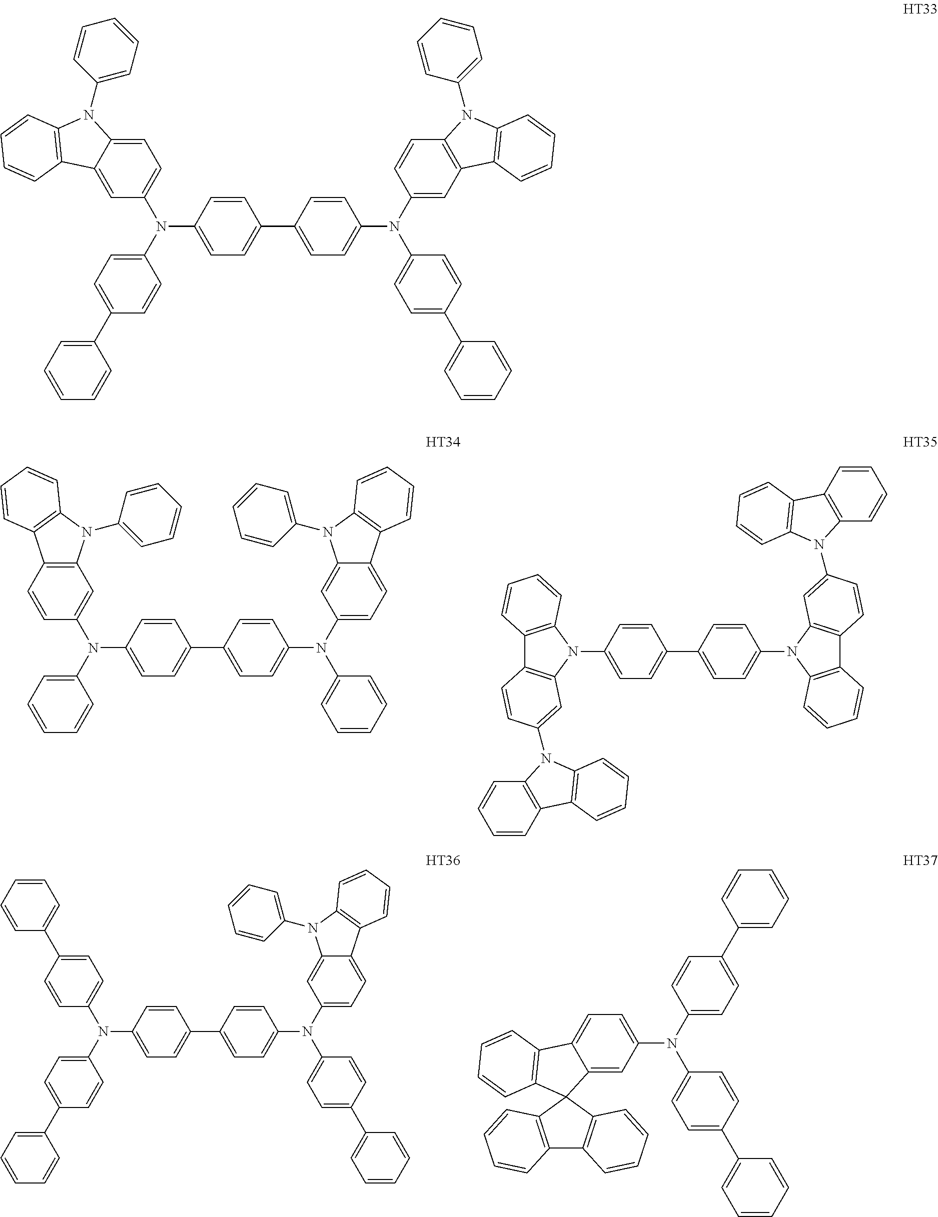

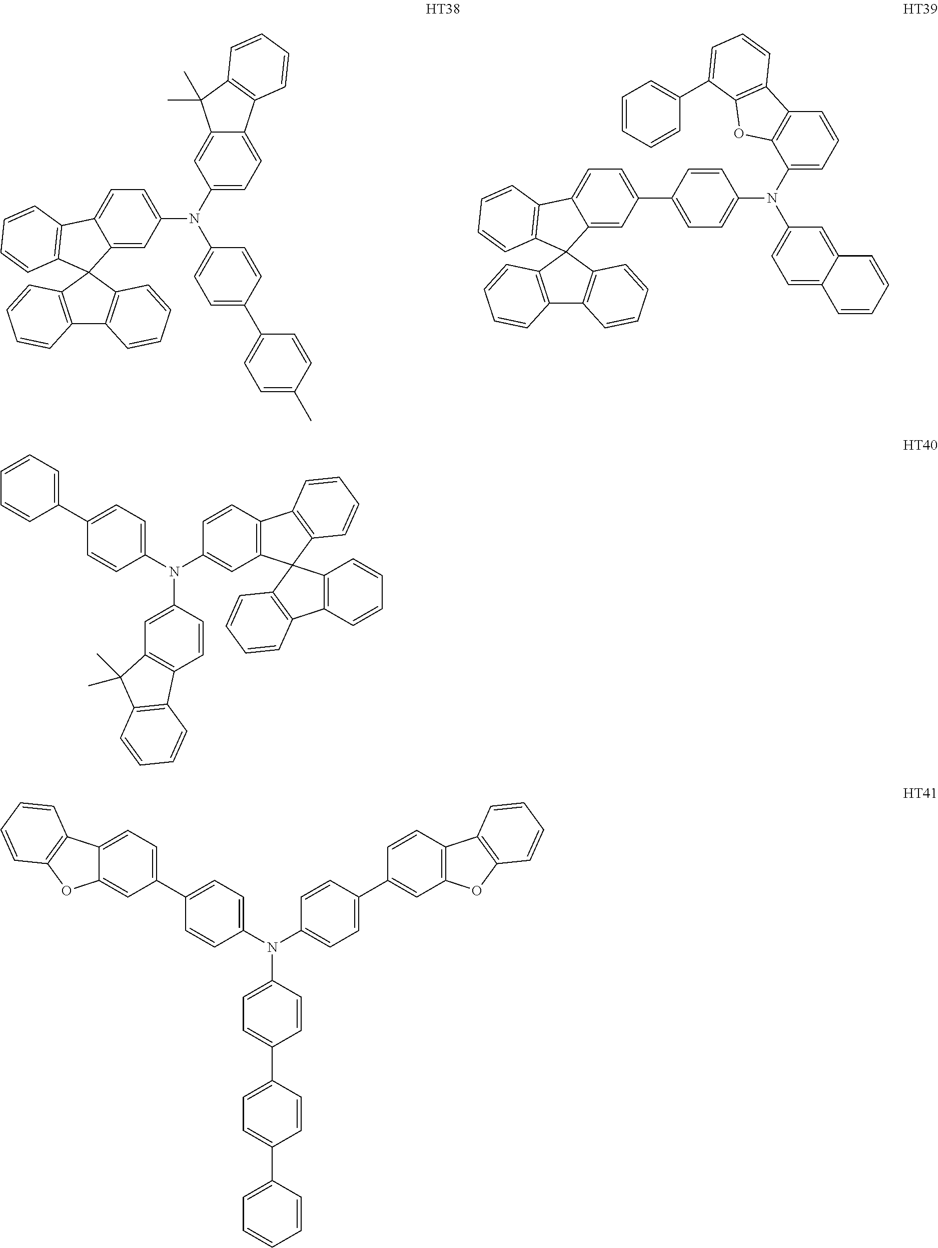 [SMILES: Cc1ccc(-c2ccc(N(c3ccc4c(c3)C(C)(C)c3ccccc3-4)c3ccc4c(c3)C3(c5ccccc5-c5ccccc53)c3ccccc3-4)cc2)cc1 ; c1ccc(-c2cccc3c2oc2c(N(c4ccc(-c5ccc6c(c5)C5(c7ccccc7-c7ccccc75)c5ccccc5-6)cc4)c4ccc5ccccc5c4)cccc23)cc1 ; CC1(C)c2ccccc2-c2ccc(N(c3ccc(-c4ccccc4)cc3)c3ccc4c(c3)C3(c5ccccc5-c5ccccc53)c3ccccc3-4)cc21 ; c1ccc(-c2ccc(-c3ccc(N(c4ccc(-c5ccc6c(c5)oc5ccccc56)cc4)c4ccc(-c5ccc6c(c5)oc5ccccc56)cc4)cc3)cc2)cc1]

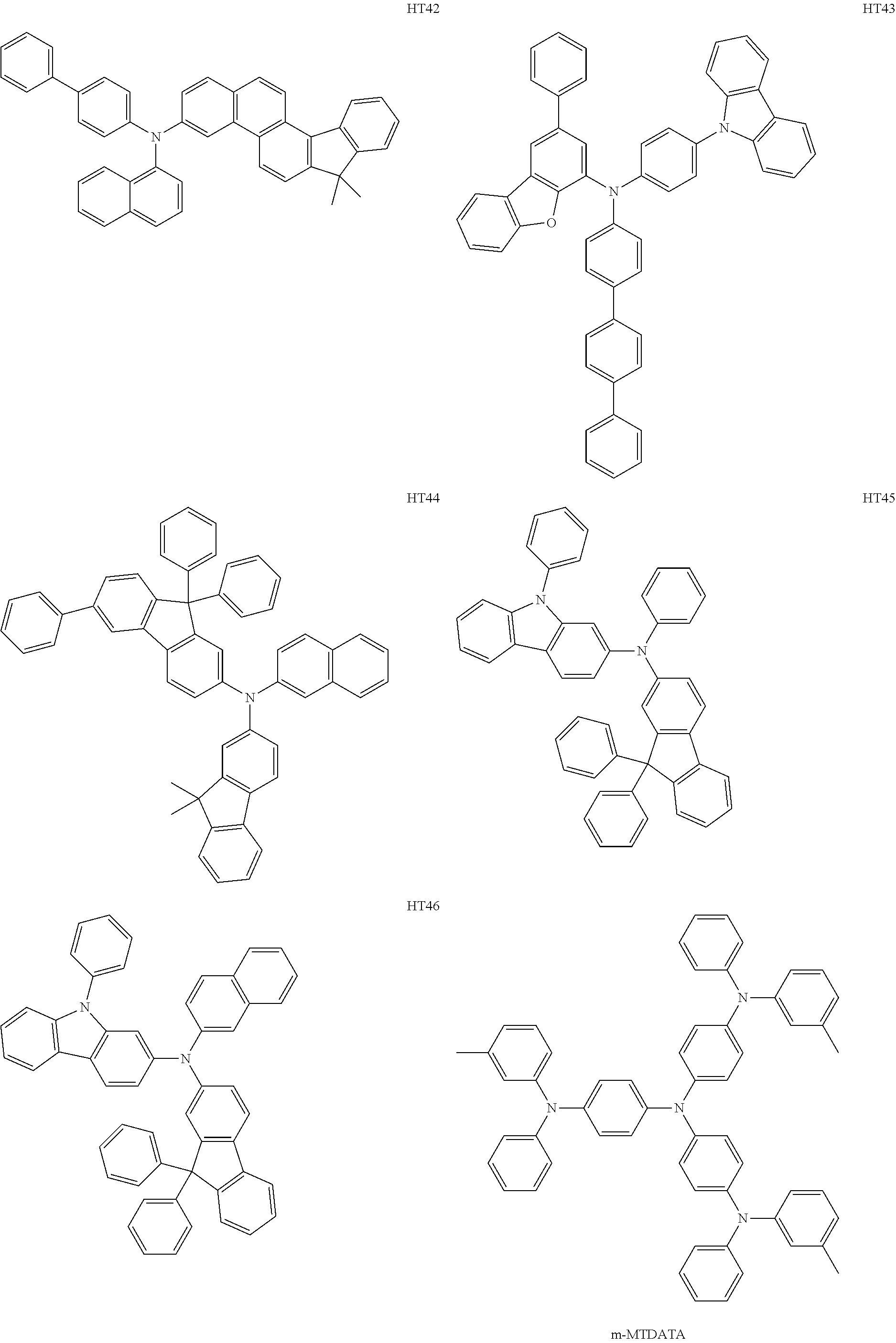

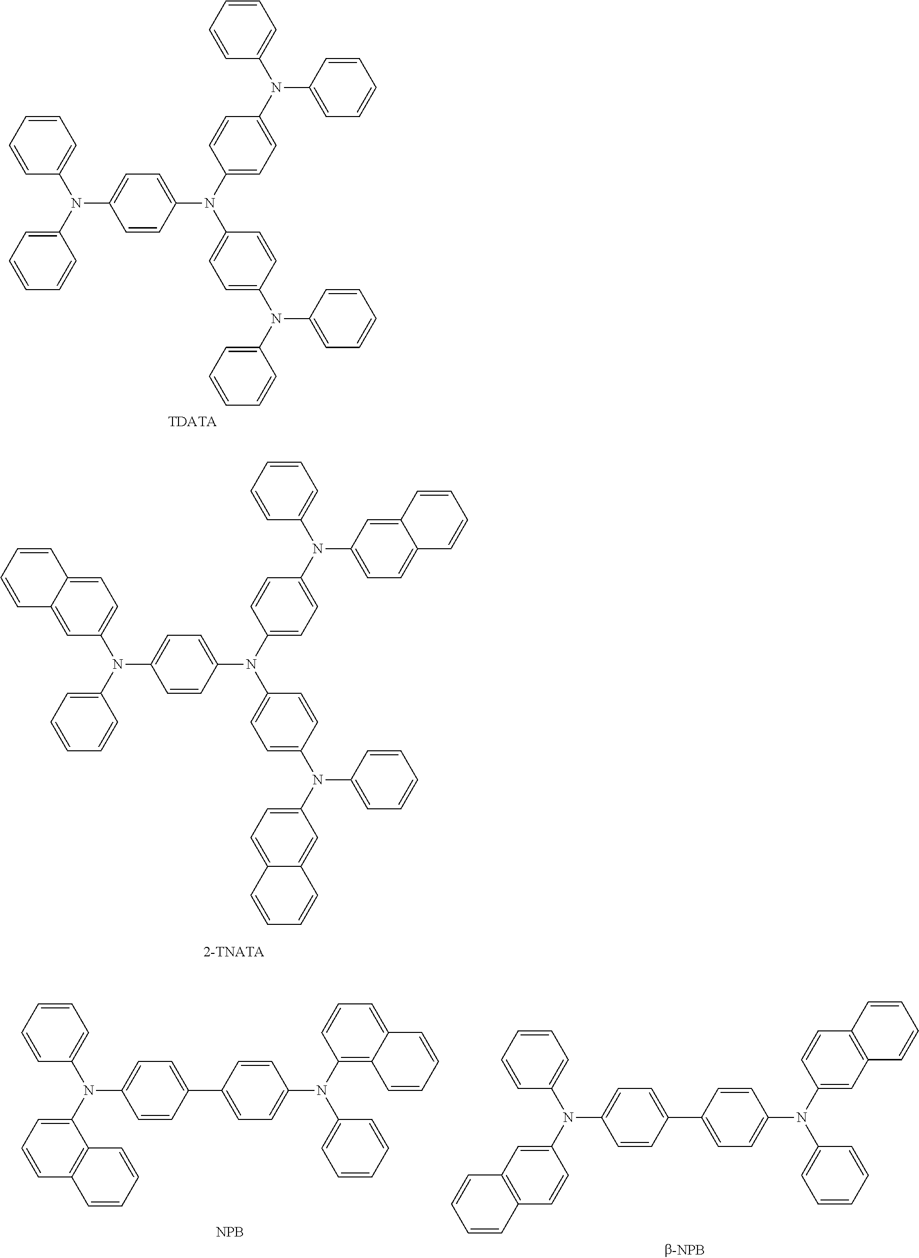 [SMILES: c1ccc(N(c2ccccc2)c2ccc(N(c3ccc(N(c4ccccc4)c4ccccc4)cc3)c3ccc(N(c4ccccc4)c4ccccc4)cc3)cc2)cc1 ; c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1 ; "TDATA" ; c1ccc(N(c2ccc(-c3ccc(N(c4ccccc4)c4cccc5ccccc45)cc3)cc2)c2cccc3ccccc23)cc1 ; "NPB" ; c1ccc(N(c2ccc(-c3ccc(N(c4ccccc4)c4ccc5ccccc5c4)cc3)cc2)c2ccc3ccccc3c2)cc1]

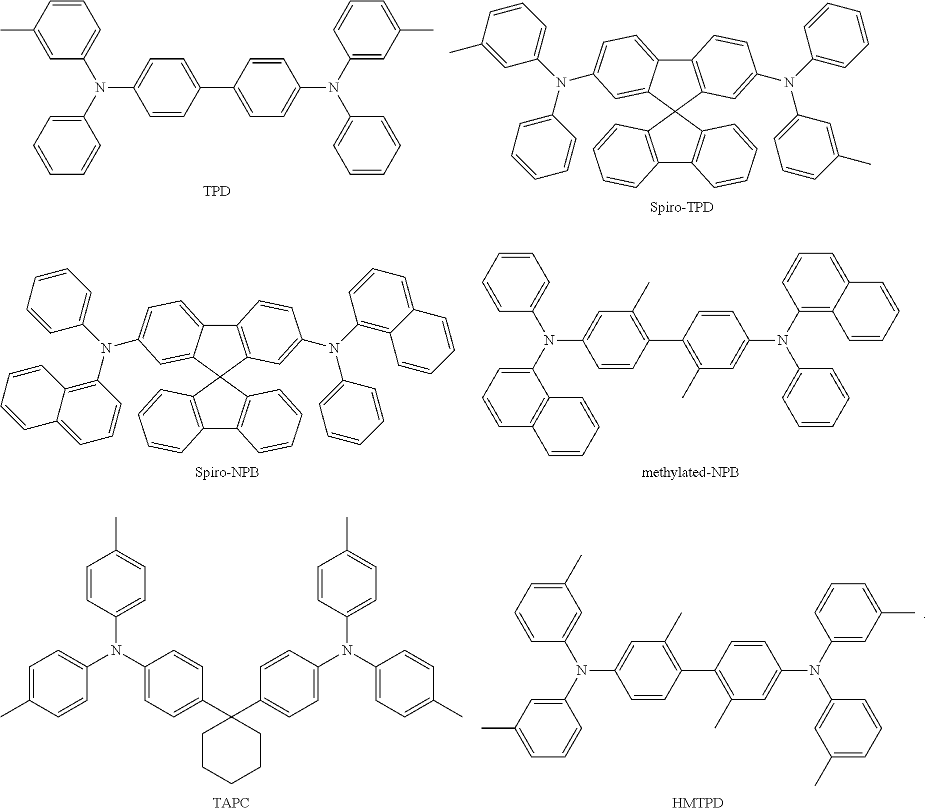

The thickness of the hole transport region may be about 50 Å to about 10,000 Å, for example, about 100 Å to about 4,000 Å. When the hole transport region includes a hole transport layer, an electron blocking layer or any combination thereof, a thickness of the hole transport layer may be about 50 Å to about 2,000 Å, for example, about 100 Å to about 1,500 Å. When the thicknesses of the hole transport region and the hole transport layer are within the ranges described above, suitable or satisfactory hole transportation characteristics may be obtained without a substantial increase in driving voltage.

The emission auxiliary layer may increase light-emission efficiency by compensating for an optical resonance distance of the wavelength of light emitted by an emission layer, and the electron blocking layer may block or reduce the leakage of electrons from an emission layer to a hole transport region. Materials that may be included in the hole transport region may be included in the emission auxiliary layer and the electron blocking layer.

p-Dopant

The hole transport region may further include, in addition to these materials, a charge-generation material for the improvement of conductive properties (e.g., electrically conductive properties). The charge-generation material may be uniformly or non-uniformly dispersed in the hole transport region (for example, in the form of a single layer consisting of a charge-generation material).

The charge-generation material may be, for example, a p-dopant.

For example, the LUMO energy of the p-dopant may be less than or equal to −3.5 eV.

According to an embodiment, the p-dopant may include a quinone derivative, a cyano group-containing compound, a compound including an element EL1 and an element EL2, or any combination thereof.

Examples of the quinone derivative may include TCNQ and F4-TCNQ.

Examples of the cyano group-containing compound may include HAT-CN and a compound represented by Formula 221.

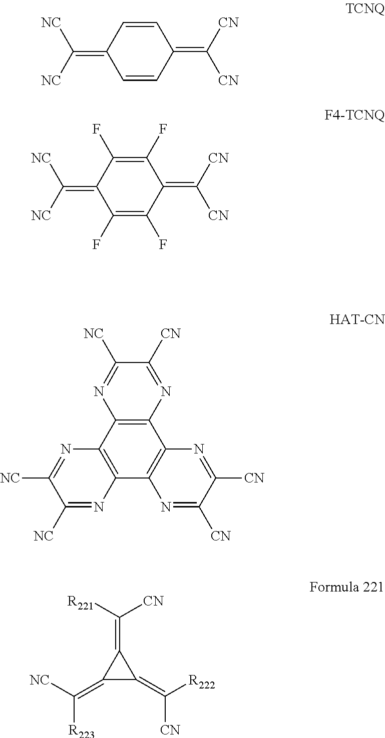

In Formula 221,
R₂₂₁ to R₂₂₃ may each independently be a C₃-C₆₀ carbocyclic group that is unsubstituted or substituted with at least one R₁₀ₐ or a C₁-C₆₀ heterocyclic group that is unsubstituted or substituted with at least one R₁₀ₐ, and
at least one of R₂₂₁ to R₂₂₃ may each independently be a C₃-C₆₀ carbocyclic group or a C₁-C₆₀ heterocyclic group, each substituted with: a cyano group; —F; —Cl; —Br; —I; a C₁-C₂₀ alkyl group substituted with a cyano group, —F, —Cl, —Br, —I, or any combination thereof; or any combination thereof.

In the compound including the element EL1 and the element EL2, the element EL1 may be a metal, a metalloid, or a combination thereof, and the element EL2 may be a non-metal, a metalloid, or a combination thereof.

Examples of the metal may include an alkali metal (for example, lithium (Li), sodium (Na), potassium (K), rubidium (Rb), cesium (Cs), and/or the like); an alkaline earth metal (for example, beryllium (Be), magnesium (Mg), calcium (Ca), strontium (Sr), barium (Ba), and/or the like); a transition metal (for example, titanium (Ti), zirconium (Zr), hafnium (Hf), vanadium (V), niobium (Nb), tantalum (Ta), chromium (Cr), molybdenum (Mo), tungsten (W), manganese (Mn), technetium (Tc), rhenium (Re), iron (Fe), ruthenium (Ru), osmium (Os), cobalt (Co), rhodium (Rh), iridium (Ir), nickel (Ni), palladium (Pd), platinum (Pt), copper (Cu), silver (Ag), gold (Au), and/or the like); a post-transition metal (for example, zinc (Zn), indium (In), tin (Sn), and/or the like); and a lanthanide metal (for example, lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), and/or the like).

Examples of the metalloid may include silicon (Si), antimony (Sb), and tellurium (Te).

Examples of the non-metal may include oxygen (O) and halogen (for example, F, Cl, Br, I, and/or the like).

Examples of the compound including the element EL1 and the element EL2 may include a metal oxide, a metal halide (for example, a metal fluoride, a metal chloride, a metal bromide, a metal iodide, and/or the like), a metalloid halide (for example, a metalloid fluoride, a metalloid chloride, a metalloid bromide, a metalloid iodide, and/or the like), a metal telluride, or any combination thereof.

Examples of the metal oxide may include a tungsten oxide (for example, WO, W₂O₃, WO₂, WO₃, W₂O₅, and/or the like), a vanadium oxide (for example, VO, V₂O₃, VO₂, V₂O₅, and/or the like), a molybdenum oxide (MoO, Mo₂O₃, MoO₂, MoO₃, Mo₂O₅, and/or the like), and a rhenium oxide (for example, ReO₃, and/or the like).

Examples of the metal halide may include an alkali metal halide, an alkaline earth metal halide, a transition metal halide, a post-transition metal halide, and a lanthanide metal halide.

Examples of the alkali metal halide may include LiF, NaF, KF, RbF, CsF, LiCl, NaCl, KCl, RbCl, CsCl, LiBr, NaBr, KBr, RbBr, CsBr, LiI, NaI, KI, RbI, and CsI.

Examples of the alkaline earth metal halide may include BeF₂, MgF₂, CaF₂, SrF₂, BaF₂, BeCl₂, MgCl₂, CaCl₂, SrCl₂, BaCl₂, BeBr₂, MgBr₂, CaBr₂, SrBr₂, BaBr₂, BeI₂, MgI₂, CaI₂, SrI₂, and BaI₂.

Examples of the transition metal halide may include a titanium halide (for example, TiF₄, TiC₄, TiBr₄, TiI₄, and/or the like), a zirconium halide (for example, ZrF₄, ZrCl₄, ZrBr4, ZrI₄, and/or the like), a hafnium halide (for example, HfF₄, HfCl₄, HfBr₄, HfI₄, and/or the like), a vanadium halide (for example, VF₃, VCl₃, VBr₃, VI₃, and/or the like), a niobium halide (for example, NbF₃, NbCl₃, NbBr₃, NbI₃, and/or the like), a tantalum halide (for example, TaF₃, TaCl₃, TaBr₃, TaI₃, and/or the like), a chromium halide (for example, CrF₃, CrO₃, CrBr₃, CrI₃, and/or the like), a molybdenum halide (for example, MoF₃, MoCl₃, MoBr₃, MoI₃, and/or the like), a tungsten halide (for example, WF₃, WCl₃, WBr₃, WI₃, and/or the like), a manganese halide (for example, MnF₂, MnCl₂, MnBr₂, MnI₂, and/or the like), a technetium halide (for example, TcF₂, TcCl₂, TcBr₂, TcI₂, and/or the like), a rhenium halide (for example, ReF₂, ReCl₂, ReBr₂, ReI₂, and/or the like), an iron halide (for example, FeF₂, FeCl₂, FeBr₂, FeI₂, and/or the like), a ruthenium halide (for example, RuF₂, RuCl₂, RuBr₂, RuI₂, and/or the like), an osmium halide (for example, OsF₂, OsCl₂, OsBr₂, OsI₂, and/or the like), a cobalt halide (for example, CoF₂, CoCl₂, CoBr₂, CoI₂, and/or the like), a rhodium halide (for example, RhF₂, RhCl₂, RhBr₂, RhI₂, and/or the like), an iridium halide (for example, IrF₂, IrCl₂, IrBr₂, IrI₂, and/or the like), a nickel halide (for example, NiF₂, NiCl₂, NiBr₂, NiI₂, and/or the like), a palladium halide (for example, PdF₂, PdCl₂, PdBr₂, PdI₂, and/or the like), a platinum halide (for example, PtF₂, PtCl₂, PtBr₂, PtI₂, and/or the like), a copper halide (for example, CuF, CuCl, CuBr, CuI, and/or the like), a silver halide (for example, AgF, AgCl, AgBr, AgI, and/or the like), and a gold halide (for example, AuF, AuCl, AuBr, AuI, and/or the like).

Examples of the post-transition metal halide may include a zinc halide (for example, ZnF₂, ZnCl₂, ZnBr₂, ZnI₂, and/or the like), an indium halide (for example, InI₃, and/or the like), and a tin halide (for example, SnI₂, and/or the like).

Examples of the lanthanide metal halide may include YbF, YbF₂, YbF₃, SmF₃, YbCl, YbCl₂, YbCl₃ SmCl₃, YbBr, YbBr₂, YbBr₃, SmBr₃, YbI, YbI₂, YbI₃, and SmI₃.

Examples of the metalloid halide may include an antimony halide (for example, SbCl₅, and/or the like).

Examples of the metal telluride may include an alkali metal telluride (for example, Li₂Te, Na₂Te, K₂Te, Rb₂Te, Cs₂Te, and/or the like), an alkaline earth metal telluride (for example, BeTe, MgTe, CaTe, SrTe, BaTe, and/or the like), a transition metal telluride (for example, TiTe₂, ZrTe₂, HfTe₂, V₂Te₃, Nb₂Te₃, Ta₂Te₃, Cr₂Te₃, Mo₂Te₃, W₂Te₃, MnTe, TcTe, ReTe, FeTe, RuTe, OsTe, CoTe, RhTe, IrTe, NiTe, PdTe, PtTe, Cu₂Te, CuTe, Ag₂Te, AgTe, Au₂Te, and/or the like), a post-transition metal telluride (for example, ZnTe, and/or the like), and a lanthanide metal telluride (for example, LaTe, CeTe, PrTe, NdTe, PmTe, EuTe, GdTe, TbTe, DyTe, HoTe, ErTe, TmTe, YbTe, LuTe, and/or the like).

### Emission Layer in Interlayer 130

When the light-emitting device 10 is a full-color light-emitting device, the emission layer may be patterned into a red emission layer, a green emission layer, and/or a blue emission layer, according to a sub-pixel. According to an embodiment, the emission layer may have a stacked structure of two or more layers of a red emission layer, a green emission layer, and a blue emission layer, in which the two or more layers contact (e.g., physically contact) each other or are separated from each other, to emit white light. In one or more embodiments, the emission layer may include two or more materials of a red light-emitting material, a green light-emitting material, and a blue light-emitting material, in which the two or more materials are mixed with each other in a single layer, to emit white light.

The emission layer may include a host and a dopant. The host may include the first host and the second host. The above dopant may include the first dopant and the second dopant described above. For example, the first dopant may include a phosphorescent dopant, and the second dopant may include a delayed fluorescent dopant.

An amount of the dopant in the emission layer may be in a range of about 0.01 parts by weight to about 35 parts by weight based on 100 parts by weight of the total host.

According to an embodiment, the emission layer may include a delayed fluorescence material. The delayed fluorescence material may act as a host or a dopant in the emission layer.

The thickness of the emission layer may be about 100 Å to about 1,000 Å, for example, about 200 Å to about 600 Å. When the thickness of the emission layer is within the range described above, excellent luminescence characteristics may be obtained without a substantial increase in driving voltage.

### Host

The host may include the first host and the second host.

The first host and the second host may each independently include a compound represented by Formula 301.

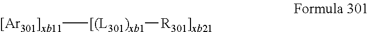

wherein, in Formula 301,
Ar₃₀₁ and L₃₀₁ may each independently be a C₃-C₆₀ carbocyclic group that is unsubstituted or substituted with at least one R₁₀ₐ or a C₁-C₆₀ heterocyclic group that is unsubstituted or substituted with at least one R₁₀ₐ,
xb11 may be 1, 2, or 3,
xb1 may be an integer from 0 to 5,
R₃₀₁ may be hydrogen, deuterium, —F, —Cl, —Br, —I, a hydroxyl group, a cyano group, a nitro group, a C₁-C₆₀ alkyl group that is unsubstituted or substituted with at least one R₁₀ₐ, a C₂-C₆₀ alkenyl group that is unsubstituted or substituted with at least one R₁₀ₐ, a C₂-C₆₀ alkynyl group that is unsubstituted or substituted with at least one R₁₀ₐ, a C₁-C₆₀ alkoxy group that is unsubstituted or substituted with at least one R₁₀ₐ, a C₃-C₆₀ carbocyclic group that is unsubstituted or substituted with at least one R₁₀ₐ, a C₁-C₆₀ heterocyclic group that is unsubstituted or substituted with at least one R₁₀ₐ, —Si(Q₃₀₁)(Q₃₀₂)(Q₃₀₃), —N(Q₃₀₁)(Q₃₀₂), —B(Q₃₀₁)(Q₃₀₂), —C(═O)(Q₃₀₁), —S(═O)₂(Q₃₀₁), or —P(═O)(Q₃₀₁)(Q₃₀₂),
xb21 may be an integer from 1 to 5, and
Q₃₀₁ to Q₃₀₃ are each as described in connection with Q₁.

According to an embodiment, when xb11 in Formula 301 is 2 or more, two or more of Ar₃₀₁ may be linked to each other via a single bond (e.g., a covalent single bond).

As another example, the first host and the second host may each independently include a compound represented by Formula 301-1 below, a compound represented by Formula 301-2 below, or any combination thereof:

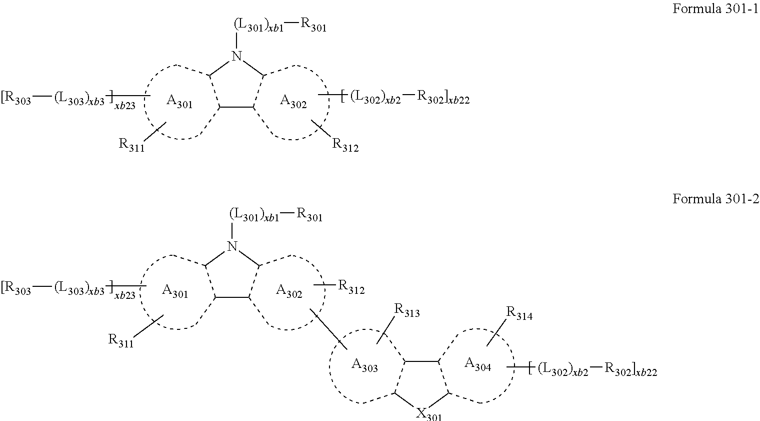

wherein, in Formulae 301-1 and 301-2,
ring A₃₀₁ to ring A₃₀₄ may each independently be a C₃-C₆₀ carbocyclic group that is unsubstituted or substituted with at least one R₁₀ₐ or a C₁-C₆₀ heterocyclic group that is unsubstituted or substituted with at least one R₁₀ₐ,
X₃₀₁ may be O, S, N-[(L₃₀₄)_{xb4}-R₃₀₄], C(R₃₀₄)(R₃₀₅), or Si(R₃₀₄)(R₃₀₅),
xb22 and xb23 may each independently be 0, 1, or 2,
L₃₀₁, xb1, and R₃₀₁ are each as described in the present specification,
L₃₀₂ to L₃₀₄ are each independently as described in connection with L₃₀₁,
xb2 to xb4 are each independently as described in connection with xb1, and
R₃₀₂ to R₃₀₅ and R₃₁₁ to R₃₁₄ are each as described in connection with R₃₀₁.

As another example, the first host and the second host may each independently include an alkaline earth metal complex, a transition metal complex, or any combination thereof. According to an embodiment, the host may include a Be complex (for example, Compound H55), an Mg complex, a Zn complex, or any combination thereof.

According to an embodiment, the first host and the second host may each independently include one selected from among Compounds H1 to H128, HT-1 to HT-4, ET-1 to ET-3, 9,10-di(2-naphthyl)anthracene (ADN), 2-methyl-9,10-bis(naphthalen-2-yl)anthracene (MADN), 9,10-di-(2-naphthyl)-2-t-butyl-anthracene (TBADN), 4,4′-bis(N-carbazolyl)-1,1′-biphenyl (CBP), 1,3-di-9-carbazolylbenzene (mCP), 1,3,5-tri(carbazol-9-yl)benzene (TCP), or any combination thereof:

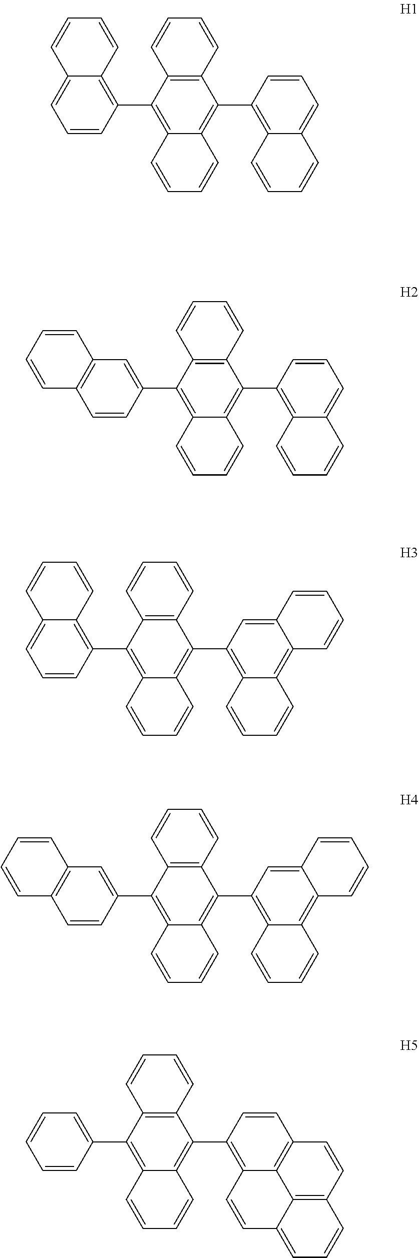

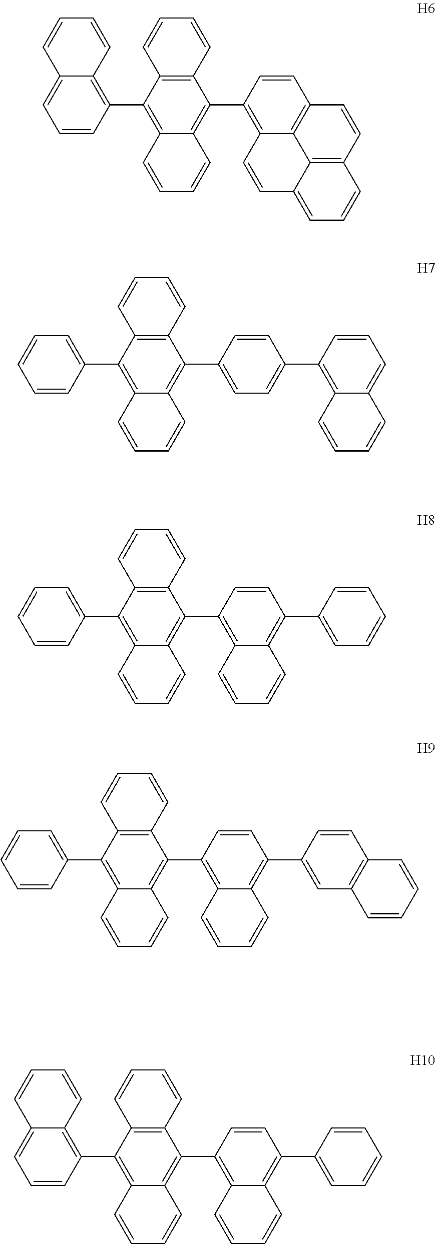

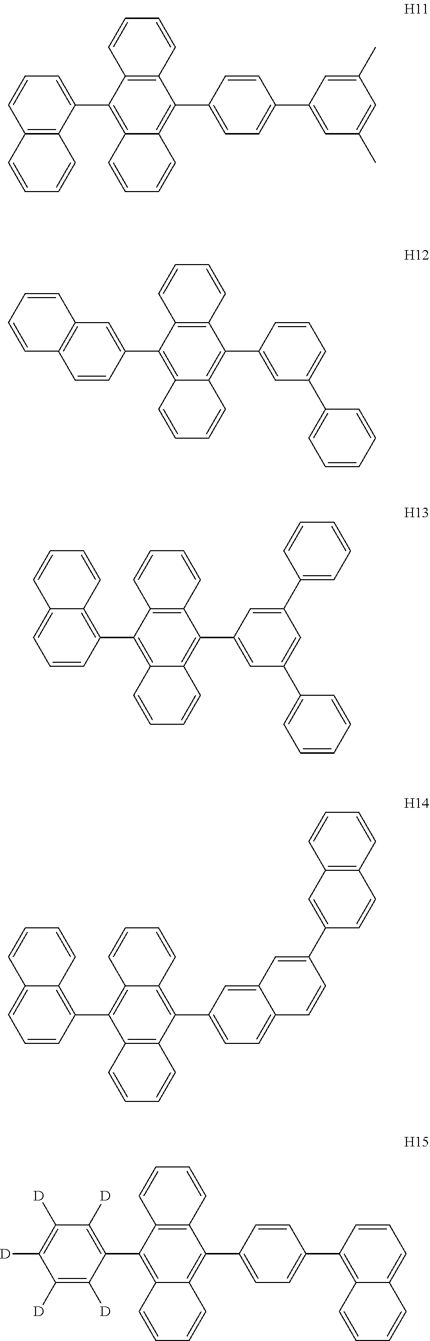

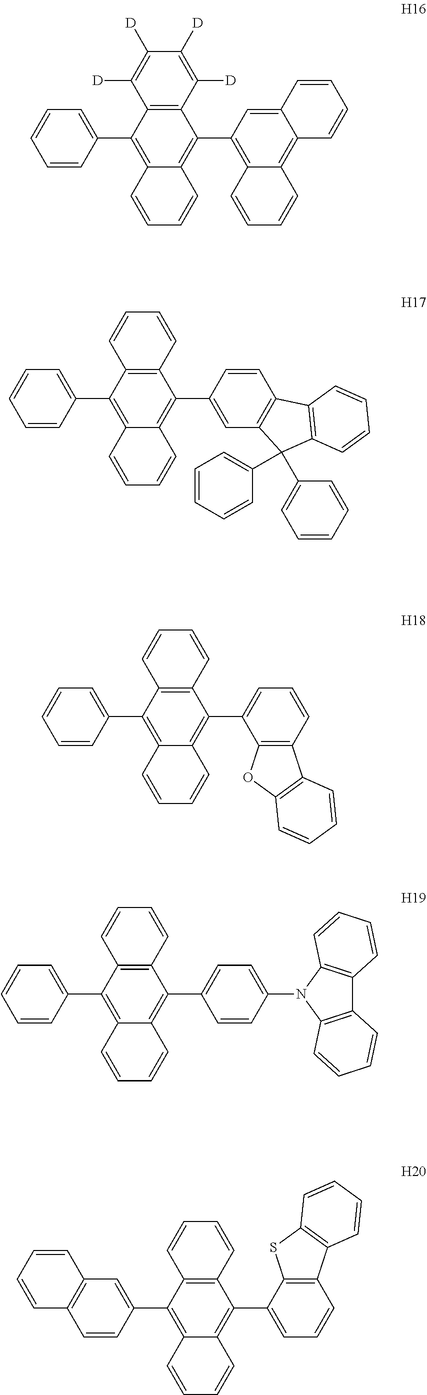

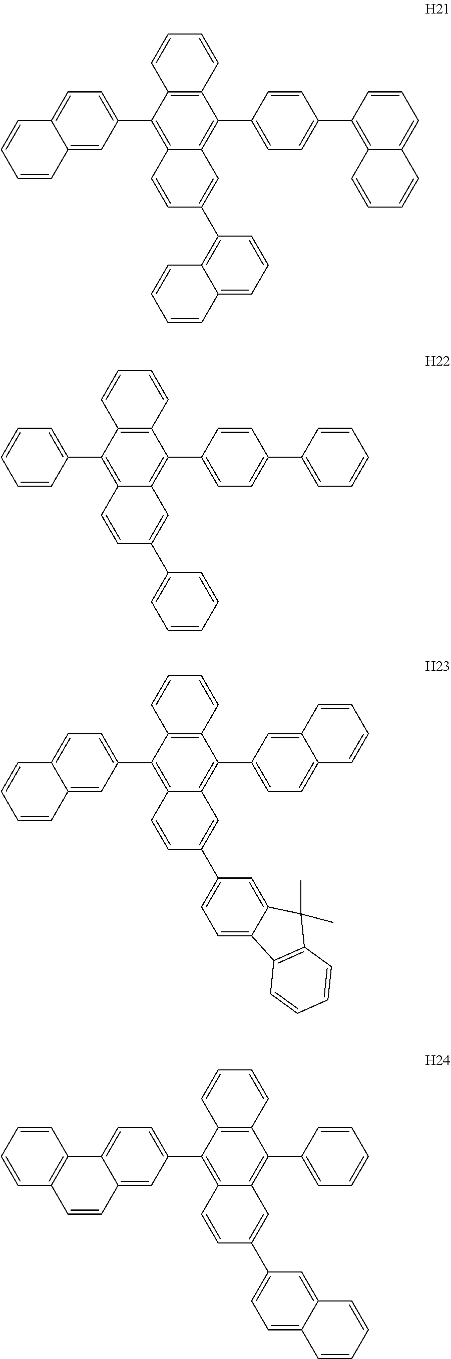

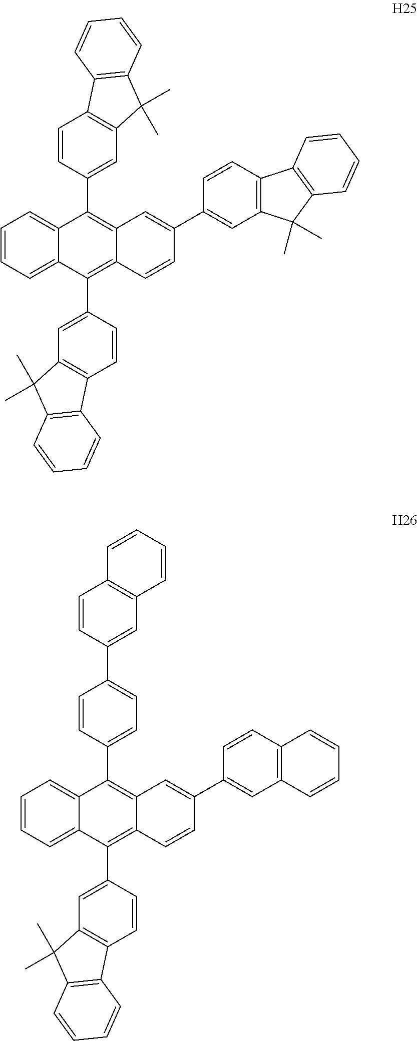

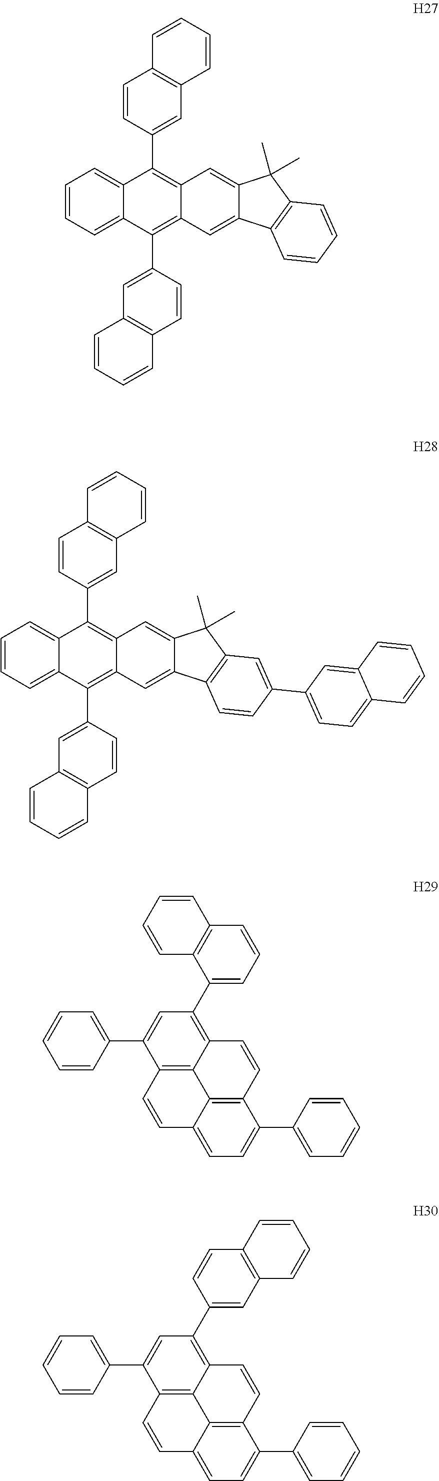

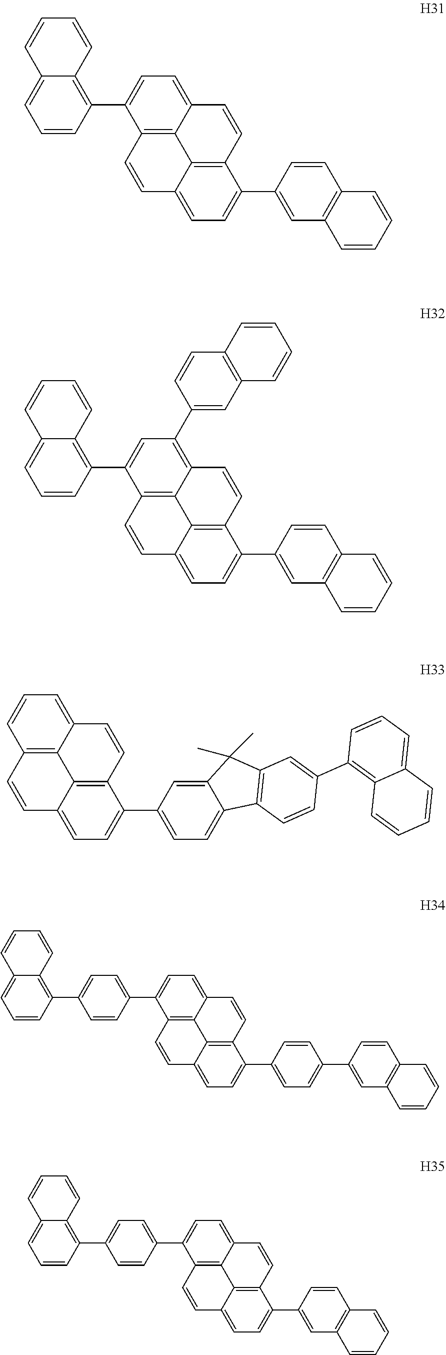

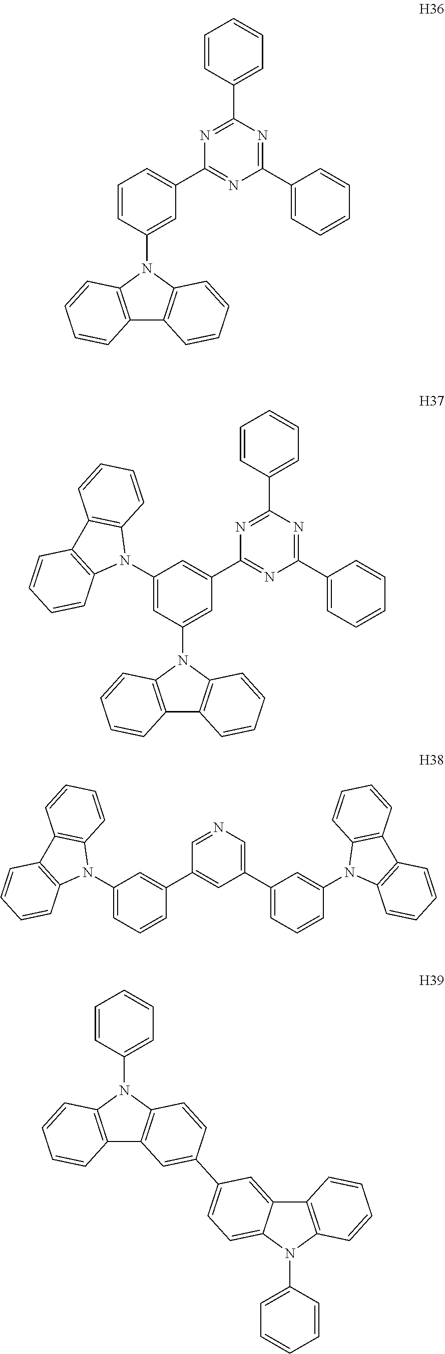 [SMILES: c1ccc(-c2nc(-c3ccccc3)nc(-c3cccc(-n4c5ccccc5c5ccccc54)c3)n2)cc1 ; c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1 ; c1ccc(-n2c3ccccc3c3cc(-c4ccc5c(c4)c4ccccc4n5-c4ccccc4)ccc32)cc1]

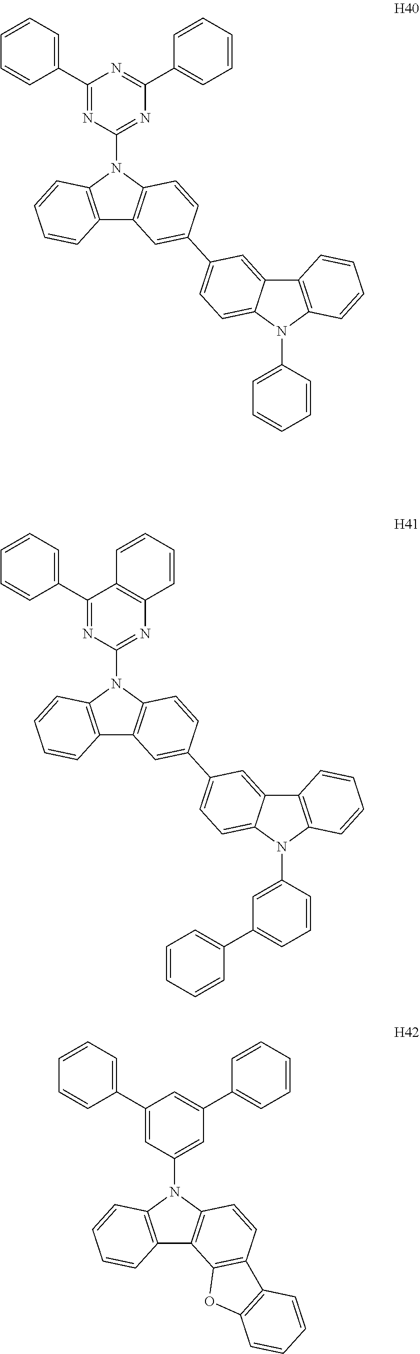 [SMILES: c1ccc(-c2nc(-c3ccccc3)nc(-n3c4ccccc4c4cc(-c5ccc6c(c5)c5ccccc5n6-c5ccccc5)ccc43)n2)cc1 ; c1ccc(-c2cccc(-n3c4ccccc4c4cc(-c5ccc6c(c5)c5ccccc5n6-c5nc(-c6ccccc6)c6ccccc6n5)ccc43)c2)cc1 ; c1ccc(-c2cc(-c3ccccc3)cc(-n3c4ccccc4c4c5oc6ccccc6c5ccc43)c2)cc1]

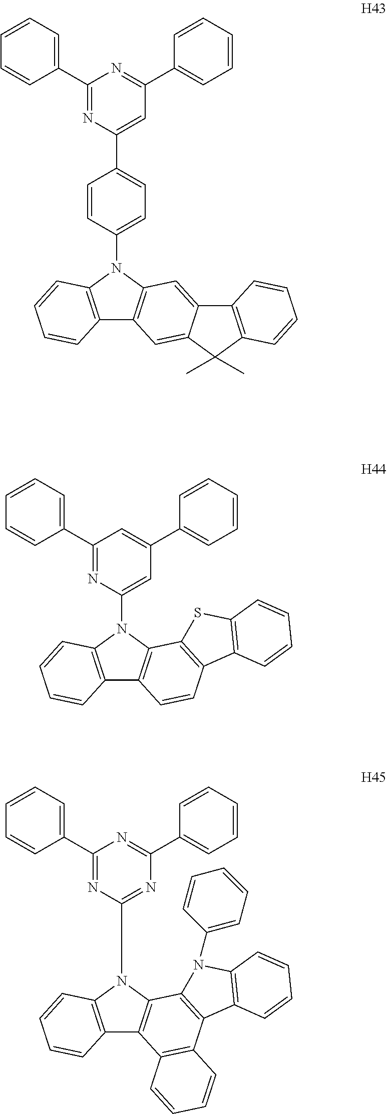 [SMILES: CC1(C)c2ccccc2-c2cc3c(cc21)c1ccccc1n3-c1ccc(-c2cc(-c3ccccc3)nc(-c3ccccc3)n2)cc1 ; c1ccc(-c2cc(-c3ccccc3)nc(-n3c4ccccc4c4ccc5c6ccccc6sc5c43)c2)cc1 ; c1ccc(-c2nc(-c3ccccc3)nc(-n3c4ccccc4c4c5ccccc5c5c6ccccc6n(-c6ccccc6)c5c43)n2)cc1]

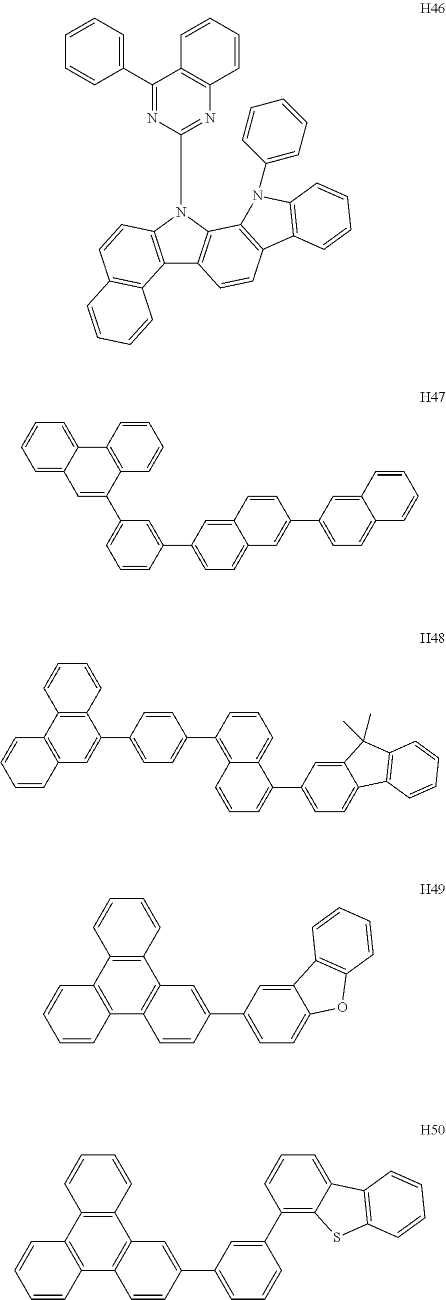

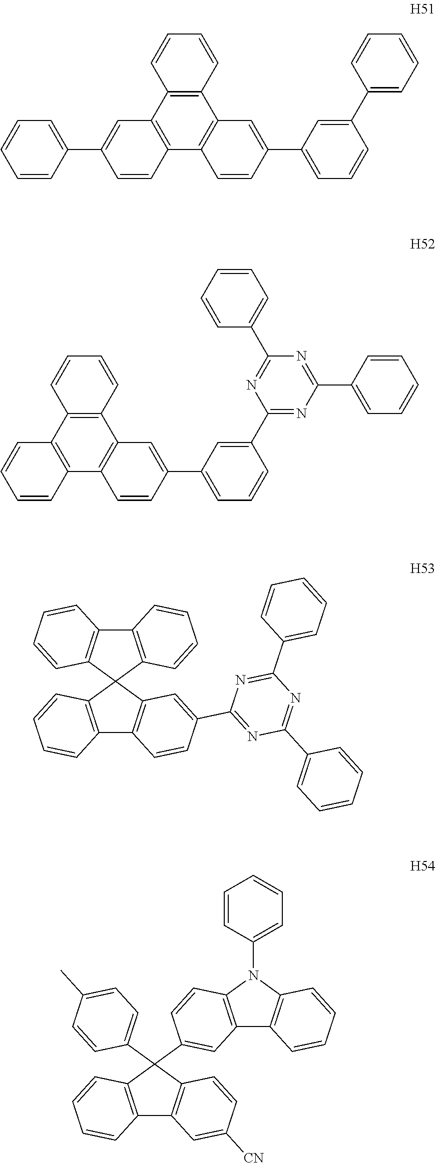

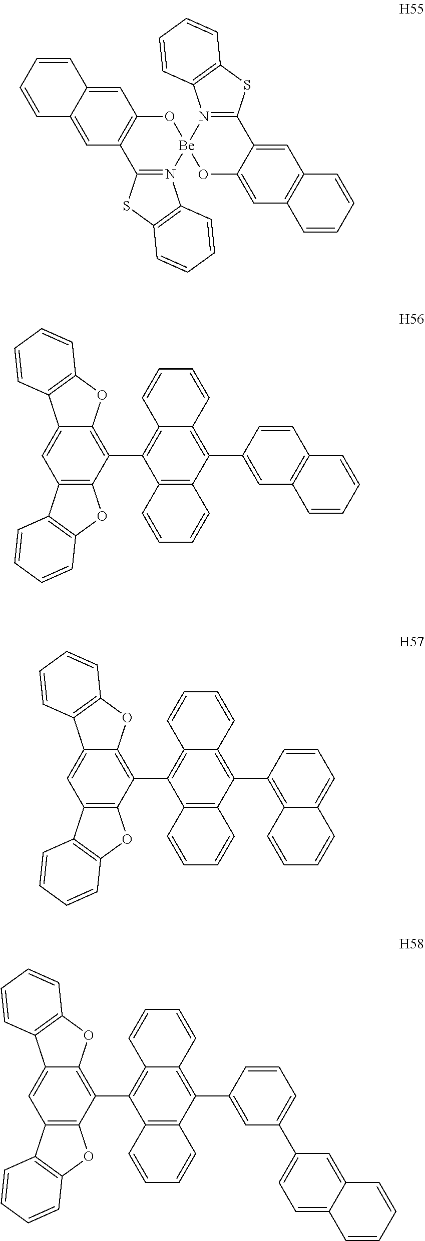

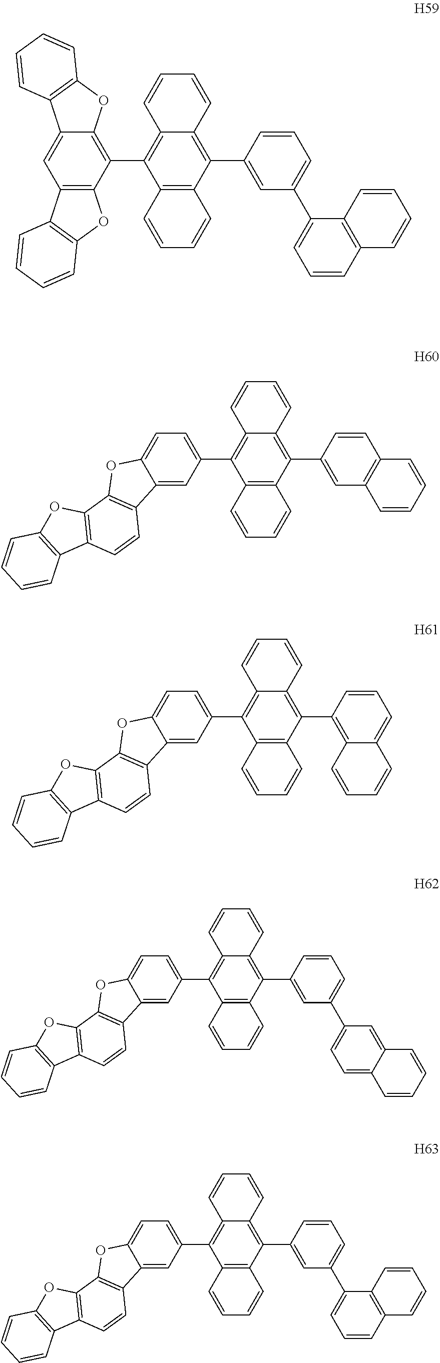 [SMILES: c1cc(-c2cccc3ccccc23)cc(-c2c3ccccc3c(-c3c4oc5ccccc5c4cc4c3oc3ccccc34)c3ccccc23)c1 ; c1ccc2cc(-c3c4ccccc4c(-c4ccc5oc6c(ccc7c8ccccc8oc76)c5c4)c4ccccc34)ccc2c1 ; c1ccc2c(-c3c4ccccc4c(-c4ccc5oc6c(ccc7c8ccccc8oc76)c5c4)c4ccccc34)cccc2c1 ; c1cc(-c2ccc3ccccc3c2)cc(-c2c3ccccc3c(-c3ccc4oc5c(ccc6c7ccccc7oc65)c4c3)c3ccccc23)c1 ; c1cc(-c2cccc3ccccc23)cc(-c2c3ccccc3c(-c3ccc4oc5c(ccc6c7ccccc7oc65)c4c3)c3ccccc23)c1]

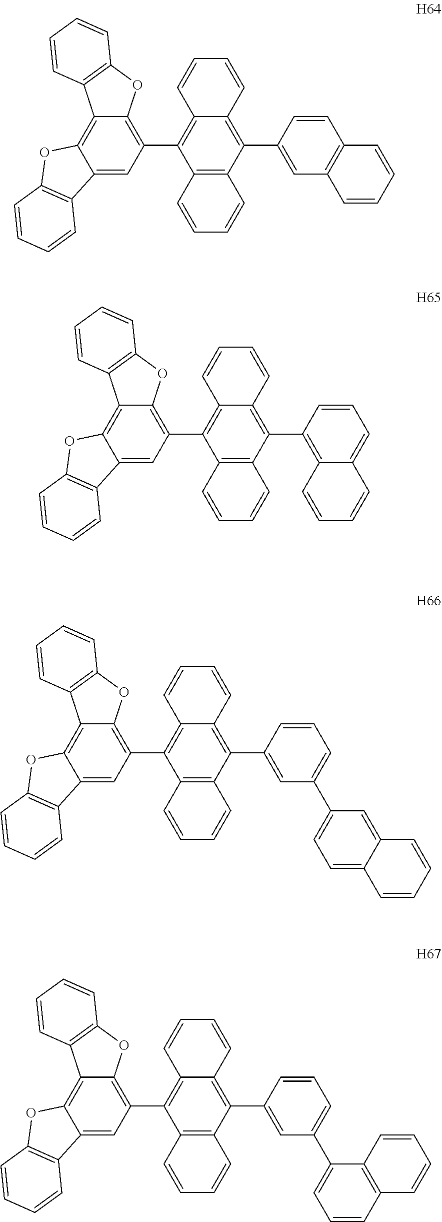 [SMILES: c1ccc2cc(-c3c4ccccc4c(-c4cc5c6ccccc6oc5c5c4oc4ccccc45)c4ccccc34)ccc2c1 ; c1ccc2c(-c3c4ccccc4c(-c4cc5c6ccccc6oc5c5c4oc4ccccc45)c4ccccc34)cccc2c1 ; c1cc(-c2ccc3ccccc3c2)cc(-c2c3ccccc3c(-c3cc4c5ccccc5oc4c4c3oc3ccccc34)c3ccccc23)c1 ; c1cc(-c2cccc3ccccc23)cc(-c2c3ccccc3c(-c3cc4c5ccccc5oc4c4c3oc3ccccc34)c3ccccc23)c1]

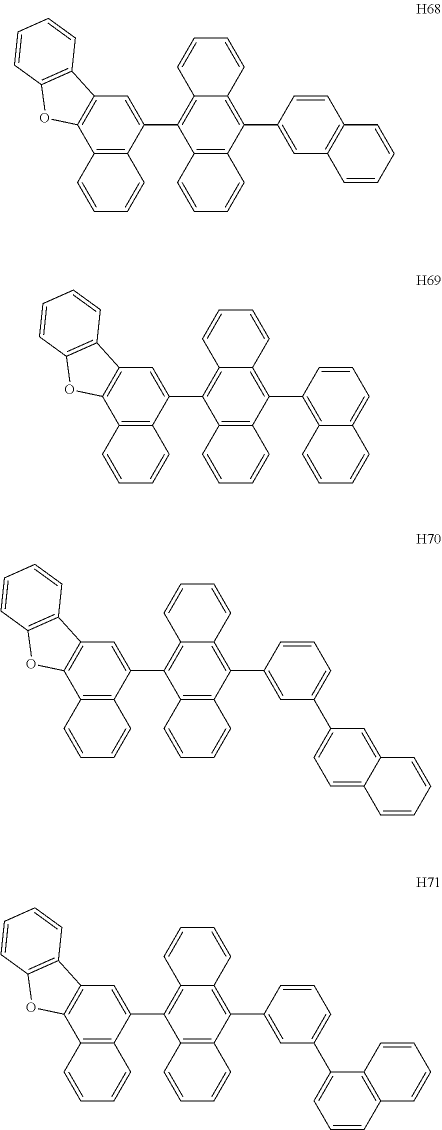 [SMILES: c1ccc2cc(-c3c4ccccc4c(-c4cc5c6ccccc6oc5c5ccccc45)c4ccccc34)ccc2c1 ; c1ccc2c(-c3c4ccccc4c(-c4cc5c6ccccc6oc5c5ccccc45)c4ccccc34)cccc2c1 ; c1cc(-c2ccc3ccccc3c2)cc(-c2c3ccccc3c(-c3cc4c5ccccc5oc4c4ccccc34)c3ccccc23)c1 ; c1cc(-c2cccc3ccccc23)cc(-c2c3ccccc3c(-c3cc4c5ccccc5oc4c4ccccc34)c3ccccc23)c1]

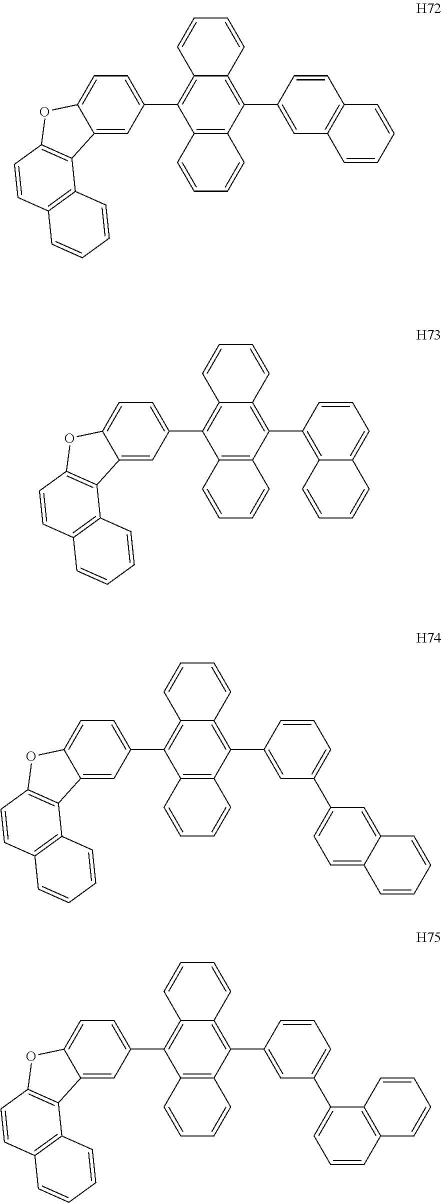 [SMILES: c1ccc2cc(-c3c4ccccc4c(-c4ccc5oc6ccc7ccccc7c6c5c4)c4ccccc34)ccc2c1 ; c1ccc2c(-c3c4ccccc4c(-c4ccc5oc6ccc7ccccc7c6c5c4)c4ccccc34)cccc2c1 ; c1cc(-c2ccc3ccccc3c2)cc(-c2c3ccccc3c(-c3ccc4oc5ccc6ccccc6c5c4c3)c3ccccc23)c1 ; c1cc(-c2cccc3ccccc23)cc(-c2c3ccccc3c(-c3ccc4oc5ccc6ccccc6c5c4c3)c3ccccc23)c1]

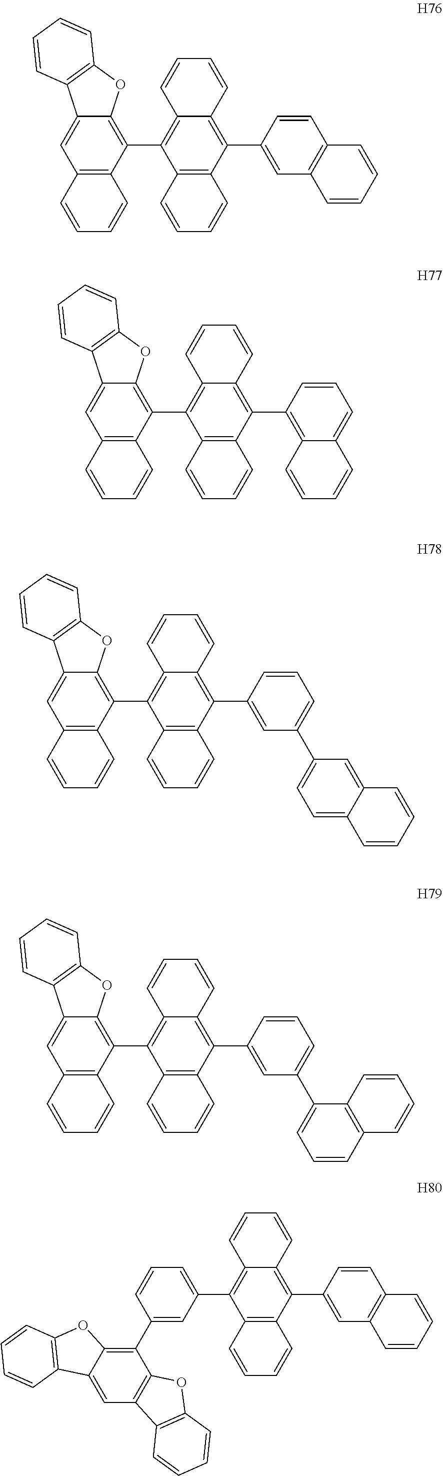 [SMILES: c1ccc2cc(-c3c4ccccc4c(-c4c5ccccc5cc5c4oc4ccccc45)c4ccccc34)ccc2c1 ; c1ccc2c(-c3c4ccccc4c(-c4c5ccccc5cc5c4oc4ccccc45)c4ccccc34)cccc2c1 ; c1cc(-c2ccc3ccccc3c2)cc(-c2c3ccccc3c(-c3c4ccccc4cc4c3oc3ccccc34)c3ccccc23)c1 ; c1cc(-c2cccc3ccccc23)cc(-c2c3ccccc3c(-c3c4ccccc4cc4c3oc3ccccc34)c3ccccc23)c1 ; c1cc(-c2c3ccccc3c(-c3ccc4ccccc4c3)c3ccccc23)cc(-c2c3oc4ccccc4c3cc3c2oc2ccccc23)c1]

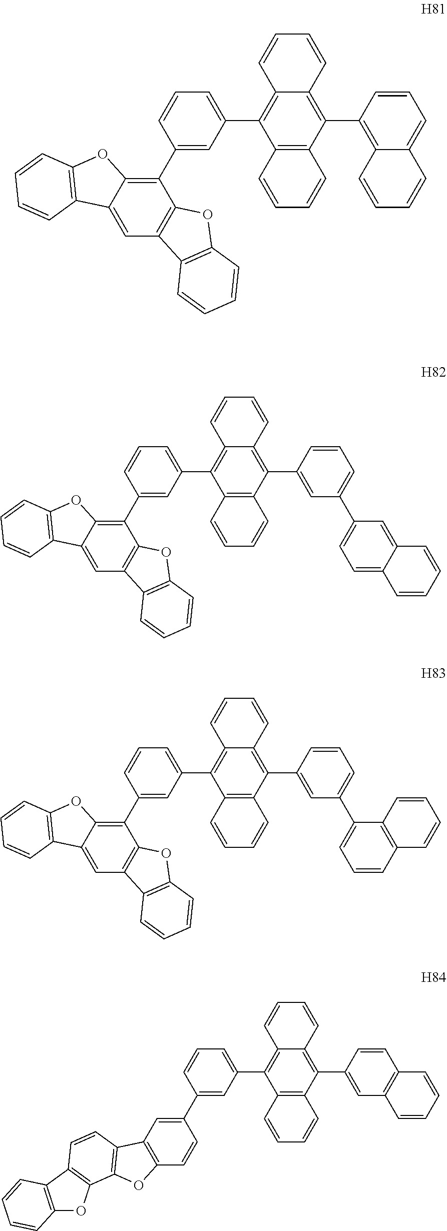 [SMILES: c1cc(-c2c3ccccc3c(-c3cccc4ccccc34)c3ccccc23)cc(-c2c3oc4ccccc4c3cc3c2oc2ccccc23)c1 ; c1cc(-c2ccc3ccccc3c2)cc(-c2c3ccccc3c(-c3cccc(-c4c5oc6ccccc6c5cc5c4oc4ccccc45)c3)c3ccccc23)c1 ; c1cc(-c2cccc3ccccc23)cc(-c2c3ccccc3c(-c3cccc(-c4c5oc6ccccc6c5cc5c4oc4ccccc45)c3)c3ccccc23)c1 ; c1cc(-c2ccc3oc4c(ccc5c6ccccc6oc54)c3c2)cc(-c2c3ccccc3c(-c3ccc4ccccc4c3)c3ccccc23)c1]

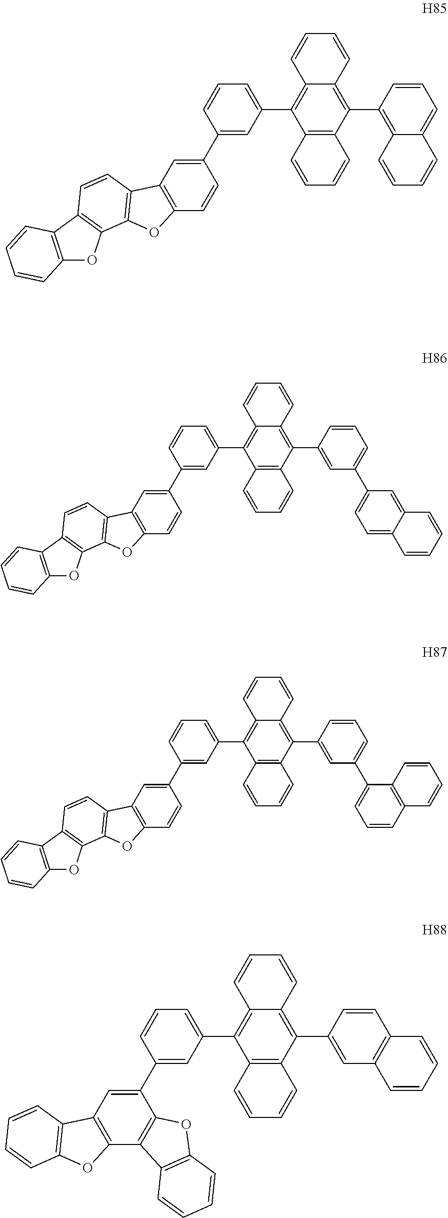 [SMILES: c1cc(-c2ccc3oc4c(ccc5c6ccccc6oc54)c3c2)cc(-c2c3ccccc3c(-c3cccc4ccccc34)c3ccccc23)c1 ; c1cc(-c2ccc3ccccc3c2)cc(-c2c3ccccc3c(-c3cccc(-c4ccc5oc6c(ccc7c8ccccc8oc76)c5c4)c3)c3ccccc23)c1 ; c1cc(-c2ccc3oc4c(ccc5c6ccccc6oc54)c3c2)cc(-c2c3ccccc3c(-c3cccc(-c4cccc5ccccc45)c3)c3ccccc23)c1 ; c1cc(-c2c3ccccc3c(-c3ccc4ccccc4c3)c3ccccc23)cc(-c2cc3c4ccccc4oc3c3c2oc2ccccc23)c1]

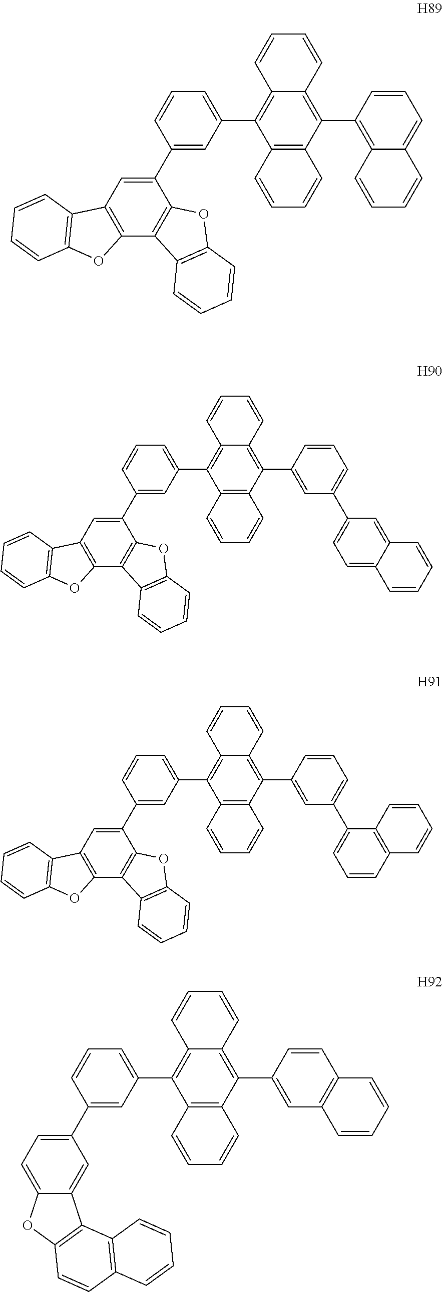 [SMILES: c1cc(-c2c3ccccc3c(-c3cccc4ccccc34)c3ccccc23)cc(-c2cc3c4ccccc4oc3c3c2oc2ccccc23)c1 ; c1cc(-c2ccc3ccccc3c2)cc(-c2c3ccccc3c(-c3cccc(-c4cc5c6ccccc6oc5c5c4oc4ccccc45)c3)c3ccccc23)c1 ; c1cc(-c2cccc3ccccc23)cc(-c2c3ccccc3c(-c3cccc(-c4cc5c6ccccc6oc5c5c4oc4ccccc45)c3)c3ccccc23)c1 ; c1cc(-c2ccc3oc4ccc5ccccc5c4c3c2)cc(-c2c3ccccc3c(-c3ccc4ccccc4c3)c3ccccc23)c1]

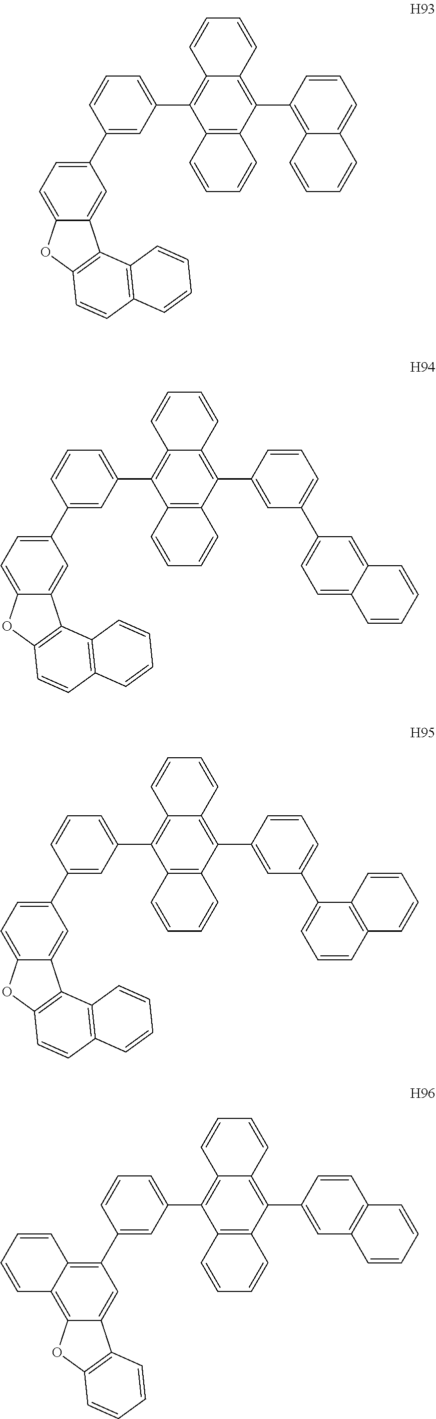 [SMILES: c1cc(-c2ccc3oc4ccc5ccccc5c4c3c2)cc(-c2c3ccccc3c(-c3cccc4ccccc34)c3ccccc23)c1 ; c1cc(-c2ccc3ccccc3c2)cc(-c2c3ccccc3c(-c3cccc(-c4ccc5oc6ccc7ccccc7c6c5c4)c3)c3ccccc23)c1 ; c1cc(-c2ccc3oc4ccc5ccccc5c4c3c2)cc(-c2c3ccccc3c(-c3cccc(-c4cccc5ccccc45)c3)c3ccccc23)c1 ; c1cc(-c2c3ccccc3c(-c3ccc4ccccc4c3)c3ccccc23)cc(-c2cc3c4ccccc4oc3c3ccccc23)c1]

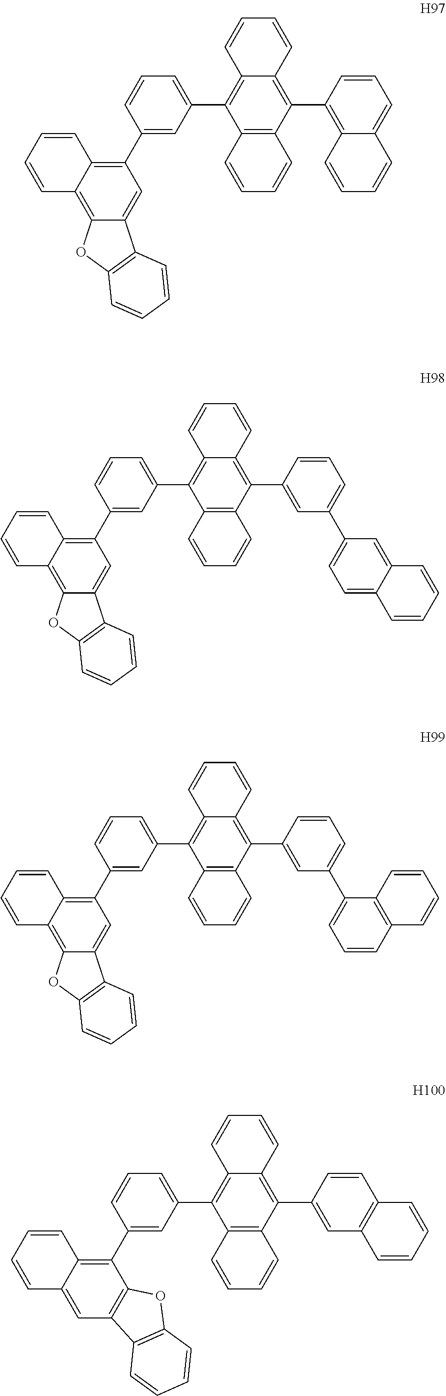 [SMILES: c1cc(-c2c3ccccc3c(-c3cccc4ccccc34)c3ccccc23)cc(-c2cc3c4ccccc4oc3c3ccccc23)c1 ; c1cc(-c2ccc3ccccc3c2)cc(-c2c3ccccc3c(-c3cccc(-c4cc5c6ccccc6oc5c5ccccc45)c3)c3ccccc23)c1 ; c1cc(-c2cccc3ccccc23)cc(-c2c3ccccc3c(-c3cccc(-c4cc5c6ccccc6oc5c5ccccc45)c3)c3ccccc23)c1 ; c1cc(-c2c3ccccc3c(-c3ccc4ccccc4c3)c3ccccc23)cc(-c2c3ccccc3cc3c2oc2ccccc23)c1]

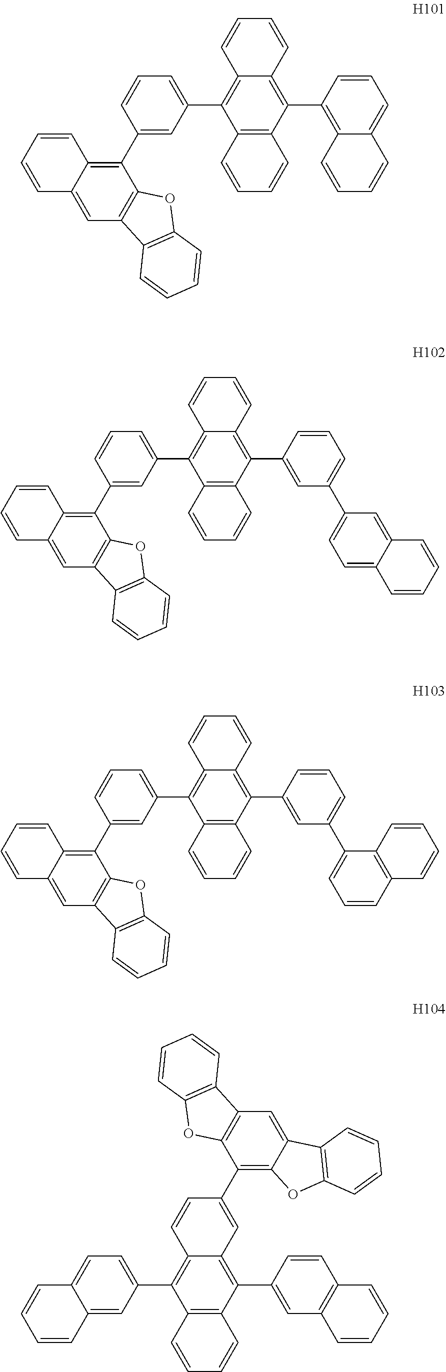 [SMILES: c1cc(-c2c3ccccc3c(-c3cccc4ccccc34)c3ccccc23)cc(-c2c3ccccc3cc3c2oc2ccccc23)c1 ; c1cc(-c2ccc3ccccc3c2)cc(-c2c3ccccc3c(-c3cccc(-c4c5ccccc5cc5c4oc4ccccc45)c3)c3ccccc23)c1 ; c1cc(-c2cccc3ccccc23)cc(-c2c3ccccc3c(-c3cccc(-c4c5ccccc5cc5c4oc4ccccc45)c3)c3ccccc23)c1 ; c1ccc2cc(-c3c4ccccc4c(-c4ccc5ccccc5c4)c4cc(-c5c6oc7ccccc7c6cc6c5oc5ccccc56)ccc34)ccc2c1]

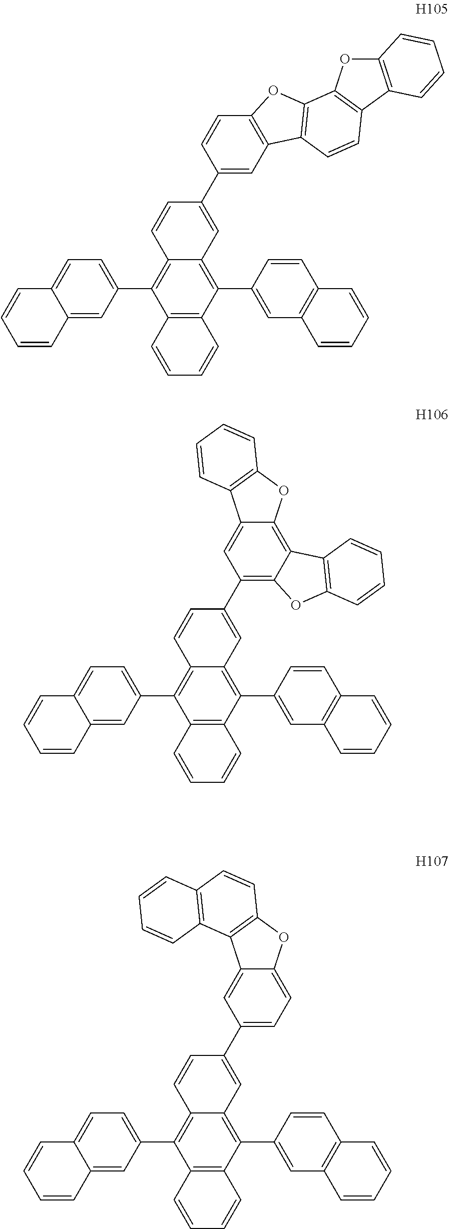 [SMILES: c1ccc2cc(-c3c4ccccc4c(-c4ccc5ccccc5c4)c4cc(-c5ccc6oc7c(ccc8c9ccccc9oc87)c6c5)ccc34)ccc2c1 ; c1ccc2cc(-c3c4ccccc4c(-c4ccc5ccccc5c4)c4cc(-c5cc6c7ccccc7oc6c6c5oc5ccccc56)ccc34)ccc2c1 ; c1ccc2cc(-c3c4ccccc4c(-c4ccc5ccccc5c4)c4cc(-c5ccc6oc7ccc8ccccc8c7c6c5)ccc34)ccc2c1]

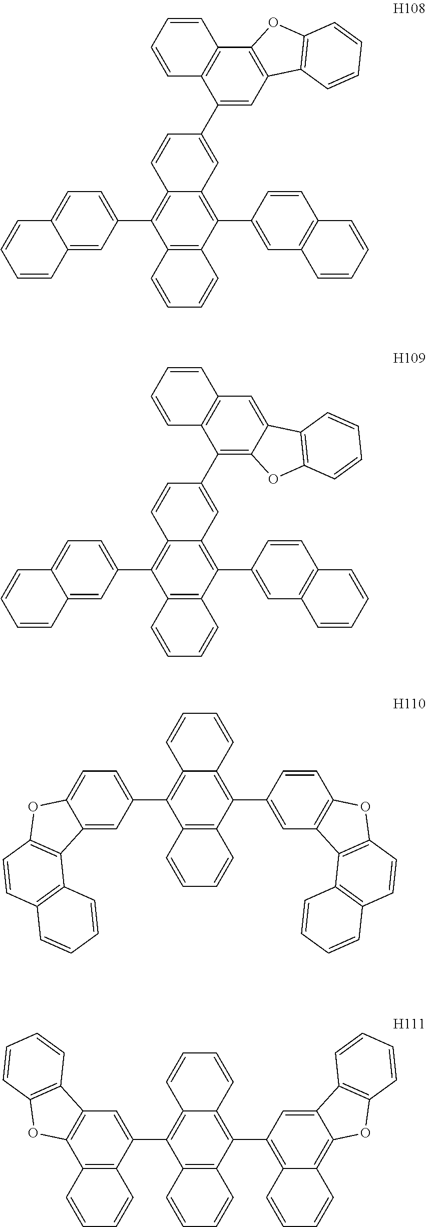 [SMILES: c1ccc2cc(-c3c4ccccc4c(-c4ccc5ccccc5c4)c4cc(-c5cc6c7ccccc7oc6c6ccccc56)ccc34)ccc2c1 ; c1ccc2cc(-c3c4ccccc4c(-c4ccc5ccccc5c4)c4cc(-c5c6ccccc6cc6c5oc5ccccc56)ccc34)ccc2c1 ; c1ccc2c(c1)ccc1oc3ccc(-c4c5ccccc5c(-c5ccc6oc7ccc8ccccc8c7c6c5)c5ccccc45)cc3c12 ; c1ccc2c(c1)oc1c3ccccc3c(-c3c4ccccc4c(-c4cc5c6ccccc6oc5c5ccccc45)c4ccccc34)cc21]

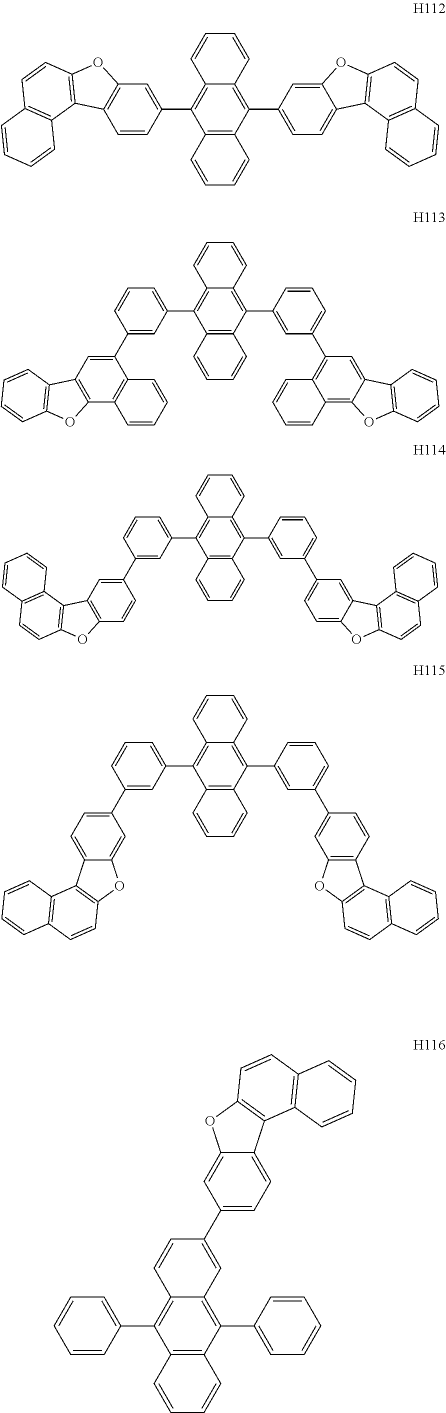 [SMILES: c1ccc2c(c1)ccc1oc3cc(-c4c5ccccc5c(-c5ccc6c(c5)oc5ccc7ccccc7c56)c5ccccc45)ccc3c12 ; c1cc(-c2c3ccccc3c(-c3cccc(-c4cc5c6ccccc6oc5c5ccccc45)c3)c3ccccc23)cc(-c2cc3c4ccccc4oc3c3ccccc23)c1 ; c1cc(-c2ccc3oc4ccc5ccccc5c4c3c2)cc(-c2c3ccccc3c(-c3cccc(-c4ccc5oc6ccc7ccccc7c6c5c4)c3)c3ccccc23)c1 ; c1cc(-c2ccc3c(c2)oc2ccc4ccccc4c23)cc(-c2c3ccccc3c(-c3cccc(-c4ccc5c(c4)oc4ccc6ccccc6c45)c3)c3ccccc23)c1 ; c1ccc(-c2c3ccccc3c(-c3ccccc3)c3cc(-c4ccc5c(c4)oc4ccc6ccccc6c45)ccc23)cc1]

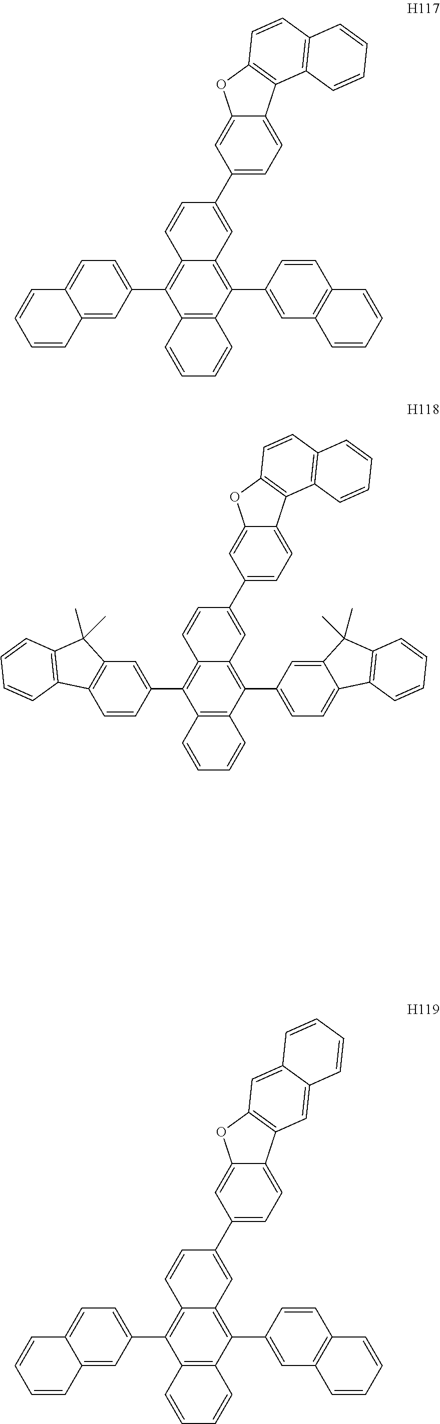 [SMILES: c1ccc2cc(-c3c4ccccc4c(-c4ccc5ccccc5c4)c4cc(-c5ccc6c(c5)oc5ccc7ccccc7c56)ccc34)ccc2c1 ; CC1(C)c2ccccc2-c2ccc(-c3c4ccccc4c(-c4ccc5c(c4)C(C)(C)c4ccccc4-5)c4cc(-c5ccc6c(c5)oc5ccc7ccccc7c56)ccc34)cc21 ; c1ccc2cc(-c3c4ccccc4c(-c4ccc5ccccc5c4)c4cc(-c5ccc6c(c5)oc5cc7ccccc7cc56)ccc34)ccc2c1]

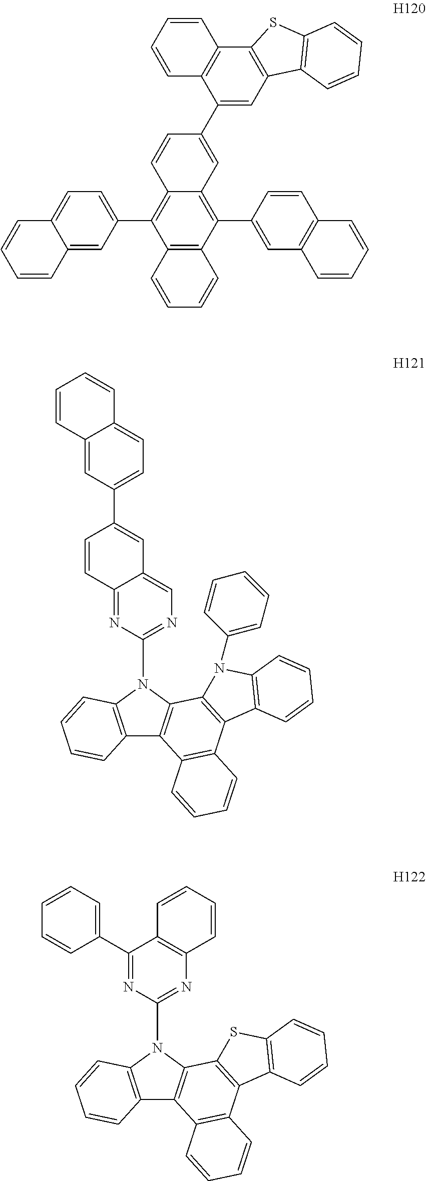 [SMILES: c1ccc2cc(-c3c4ccccc4c(-c4ccc5ccccc5c4)c4cc(-c5cc6c7ccccc7sc6c6ccccc56)ccc34)ccc2c1 ; c1ccc(-n2c3ccccc3c3c4ccccc4c4c5ccccc5n(-c5ncc6cc(-c7ccc8ccccc8c7)ccc6n5)c4c32)cc1 ; c1ccc(-c2nc(-n3c4ccccc4c4c5ccccc5c5c6ccccc6sc5c43)nc3ccccc23)cc1]

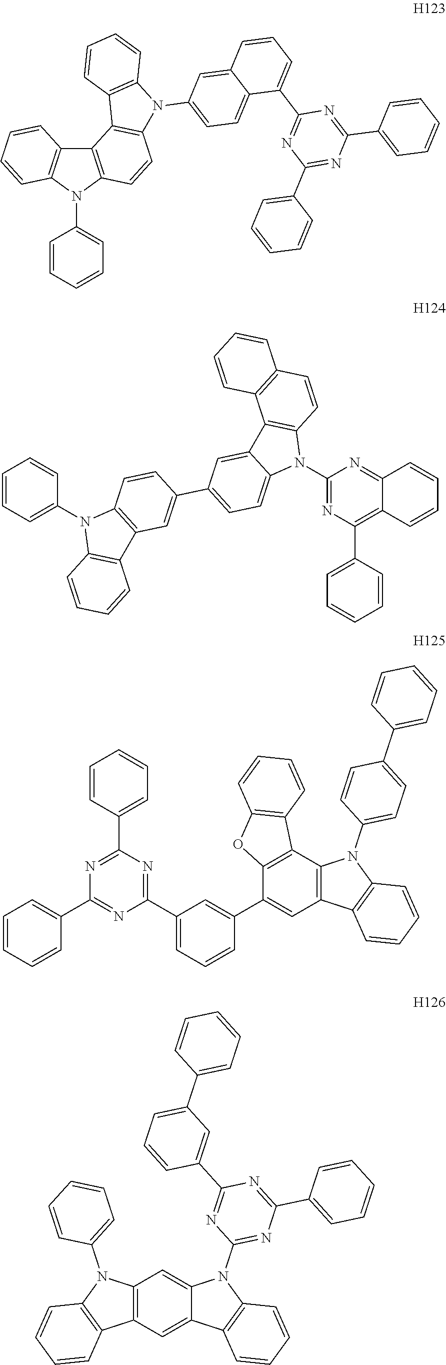 [SMILES: c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1 ; c1ccc(-c2ccc(-n3c4ccccc4c4cc(-c5cccc(-c6nc(-c7ccccc7)nc(-c7ccccc7)n6)c5)c5oc6ccccc6c5c43)cc2)cc1 ; c1ccc(-c2cccc(-c3nc(-c4ccccc4)nc(-n4c5ccccc5c5cc6c7ccccc7n(-c7ccccc7)c6cc54)n3)c2)cc1]

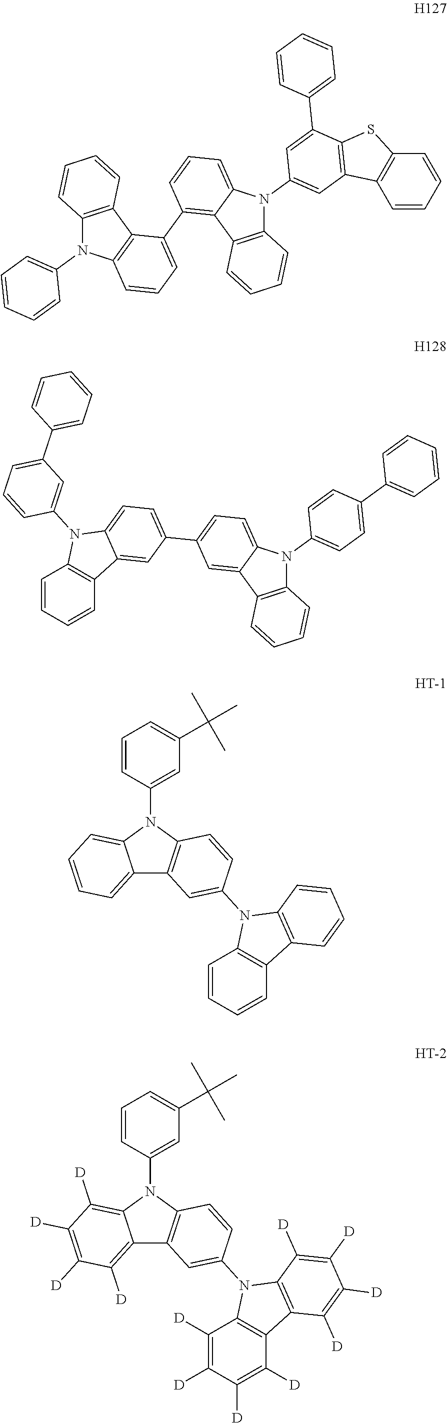 [SMILES: c1ccc(-c2cc(-n3c4ccccc4c4c(-c5cccc6c5c5ccccc5n6-c5ccccc5)cccc43)cc3c2sc2ccccc23)cc1 ; c1ccc(-c2ccc(-n3c4ccccc4c4cc(-c5ccc6c(c5)c5ccccc5n6-c5cccc(-c6ccccc6)c5)ccc43)cc2)cc1 ; CC(C)(C)c1cccc(-n2c3ccccc3c3cc(-n4c5ccccc5c5ccccc54)ccc32)c1 ; [2H]c1c([2H])c([2H])c2c(c1[2H])c1cc(-n3c4c([2H])c([2H])c([2H])c([2H])c4c4c([2H])c([2H])c([2H])c([2H])c43)ccc1n2-c1cccc(C(C)(C)C)c1]

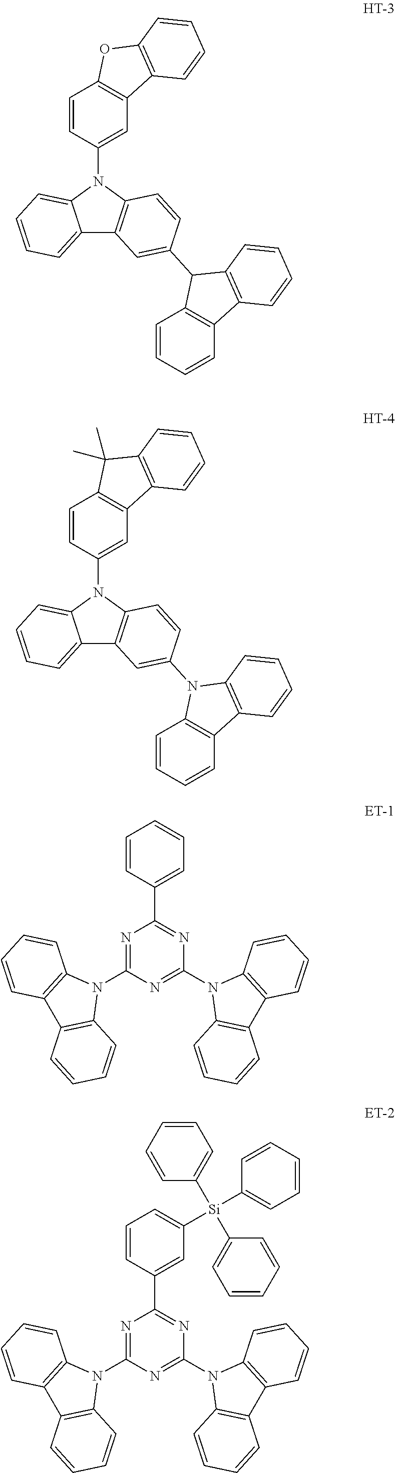

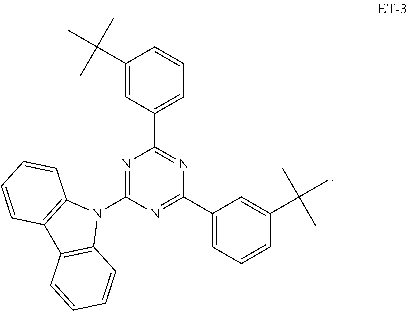 [SMILES: CC(C)(C)c1cccc(-c2nc(-c3cccc(C(C)(C)C)c3)nc(-n3c4ccccc4c4ccccc43)n2)c1]

### Phosphorescent Dopant

Reference may be made to the first dopant described above for the phosphorescent dopant.

According to an embodiment, the first dopant may be present in an amount of about 1.0 parts by weight to about 30 parts by weight (based on 100 parts by weight of the total host). In embodiments where the amount of the first dopant is within this range, the light-emitting device may have excellent efficiency and lifespan.

The first dopant may include, for example, one selected from among Compounds PD26 to PD39, PS-1, and PS-2:

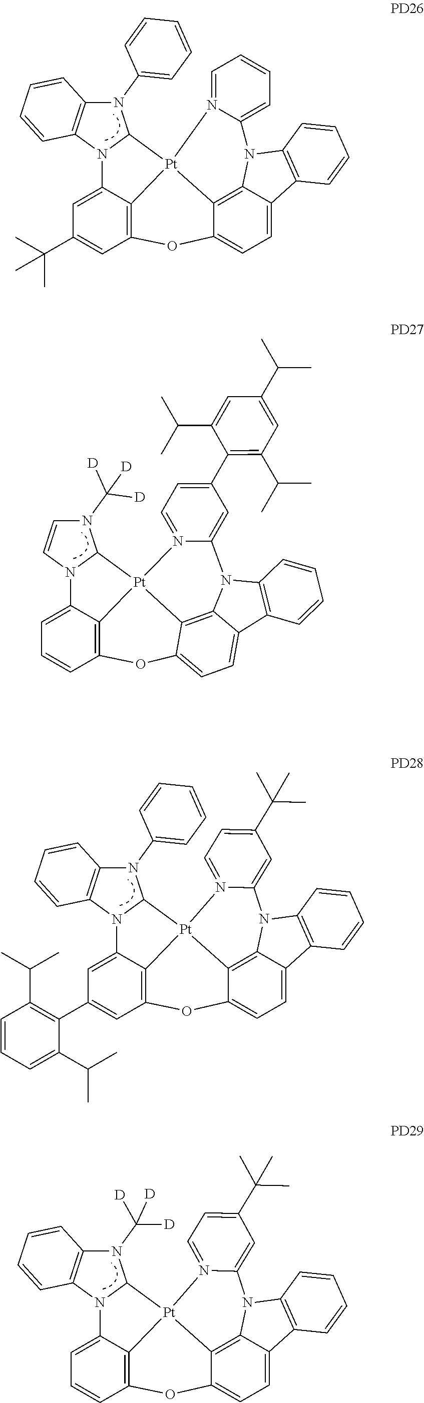

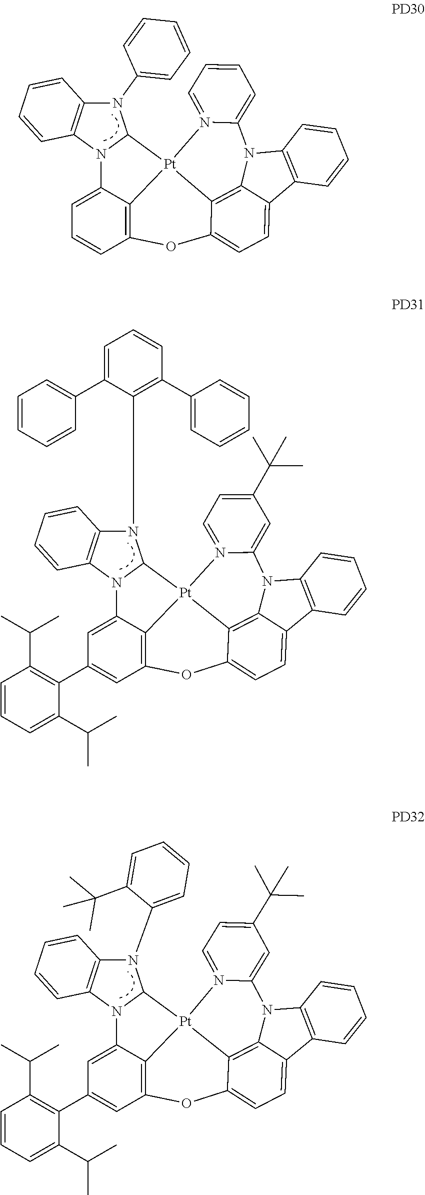

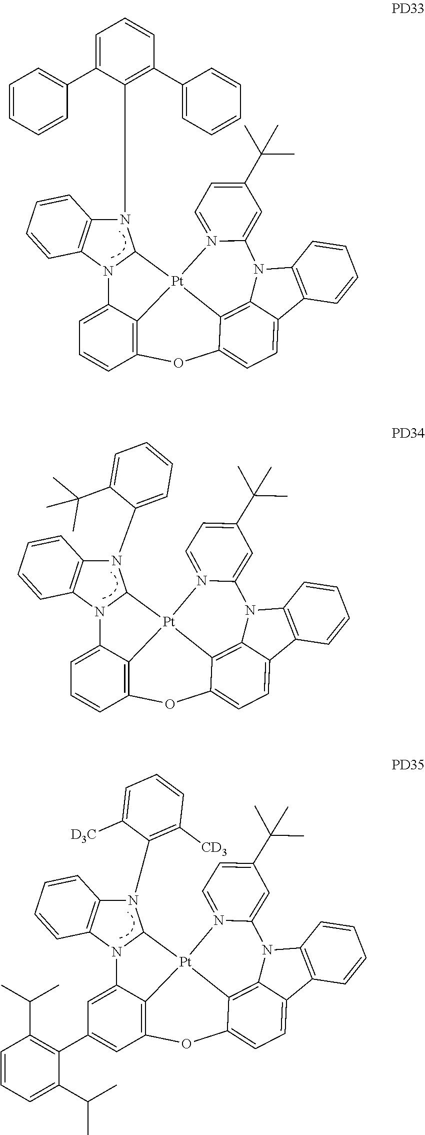

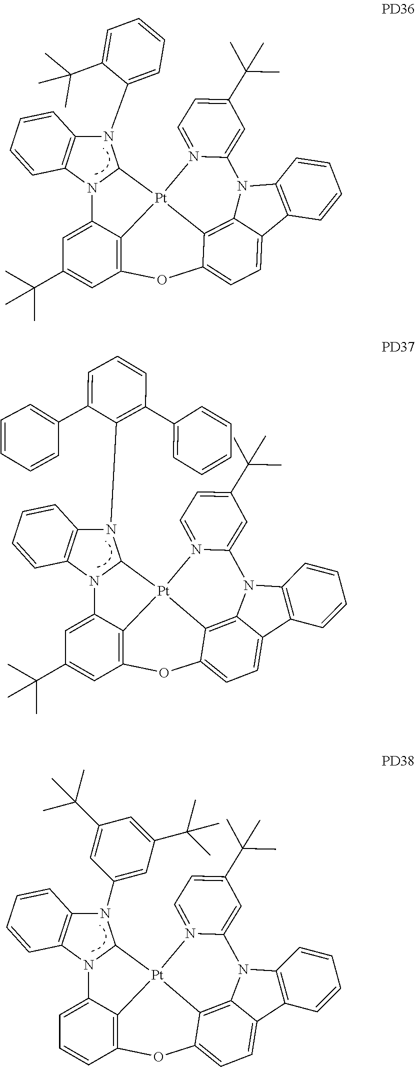

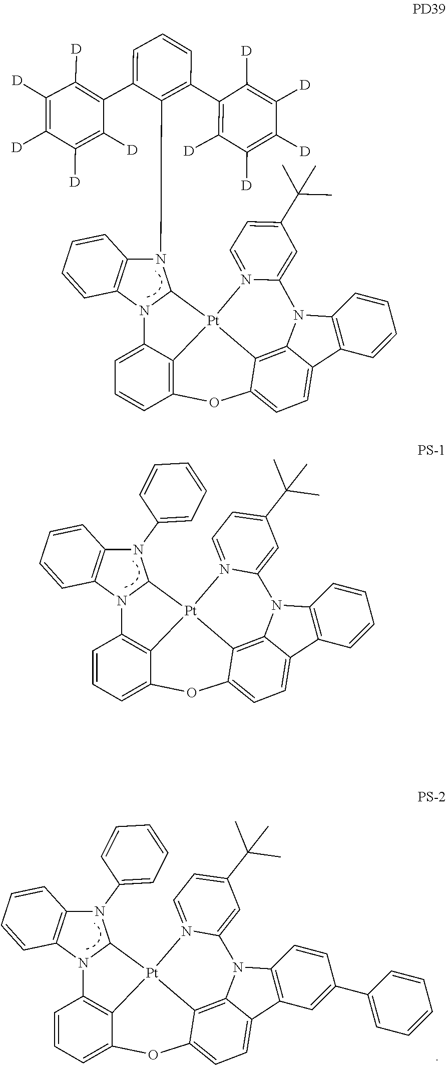

### Delayed Fluorescence Material

Reference may be made to the second dopant described above for the delayed fluorescence material.

According to an embodiment, the amount of the second dopant may be about 1.0 parts by weight to about 7.0 parts by weight (based on 100 parts by weight of total host). In embodiments where the amount of the second dopant is within this range, the light-emitting device may have excellent efficiency and lifespan.

The second dopant may include, for example, any one selected from among the following compounds:

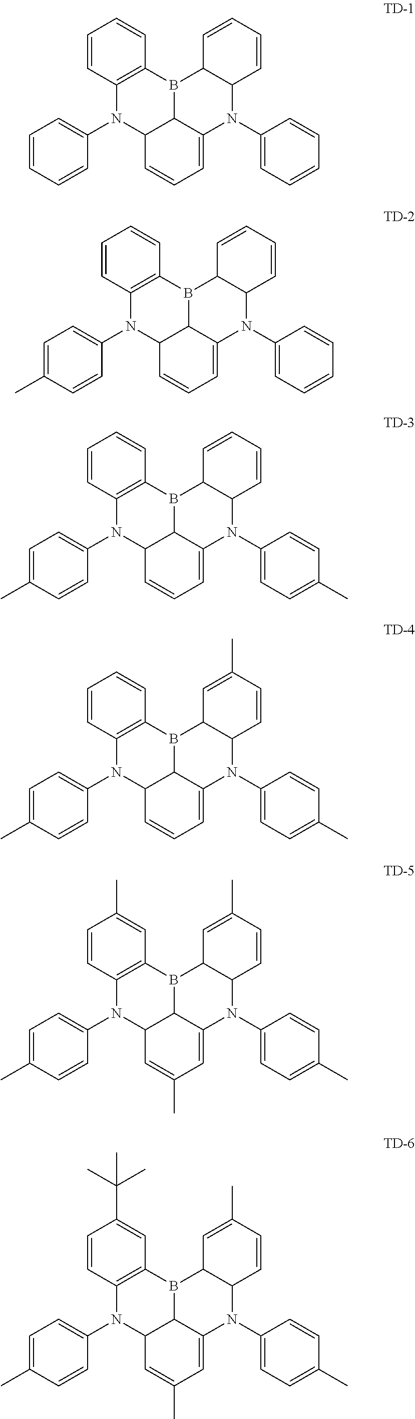

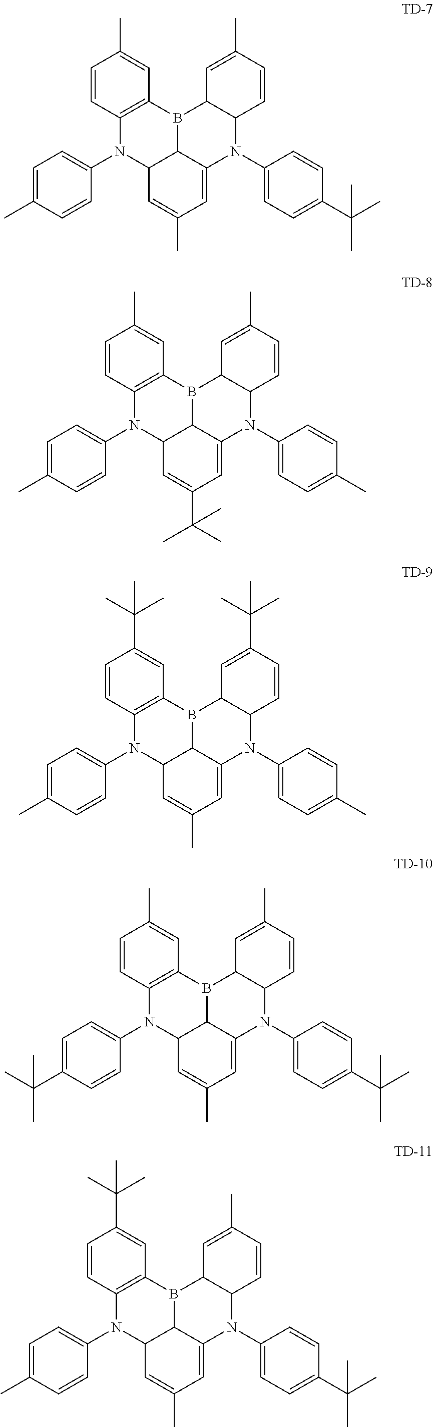

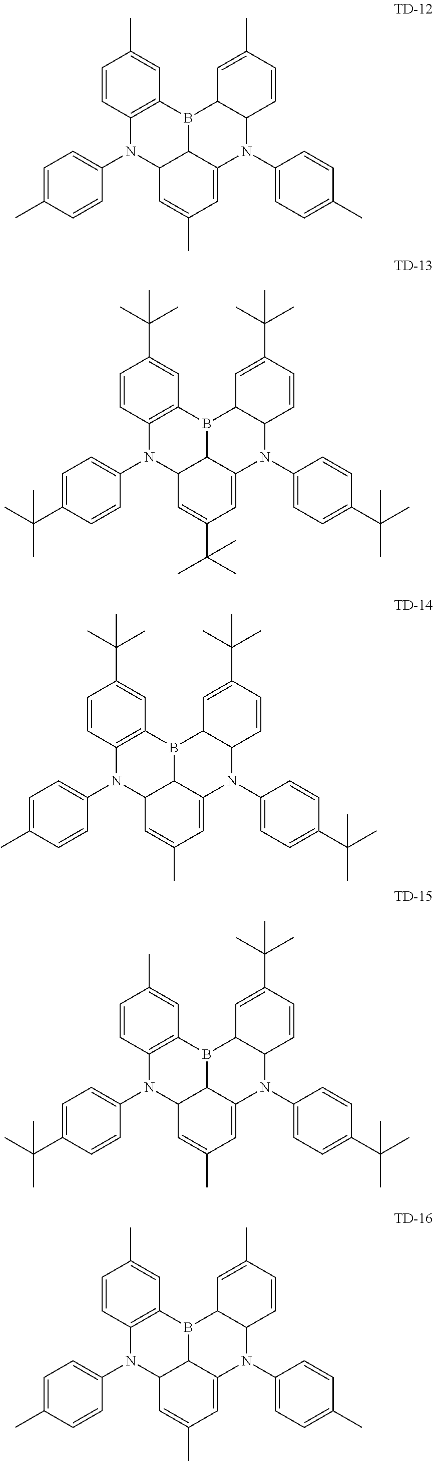

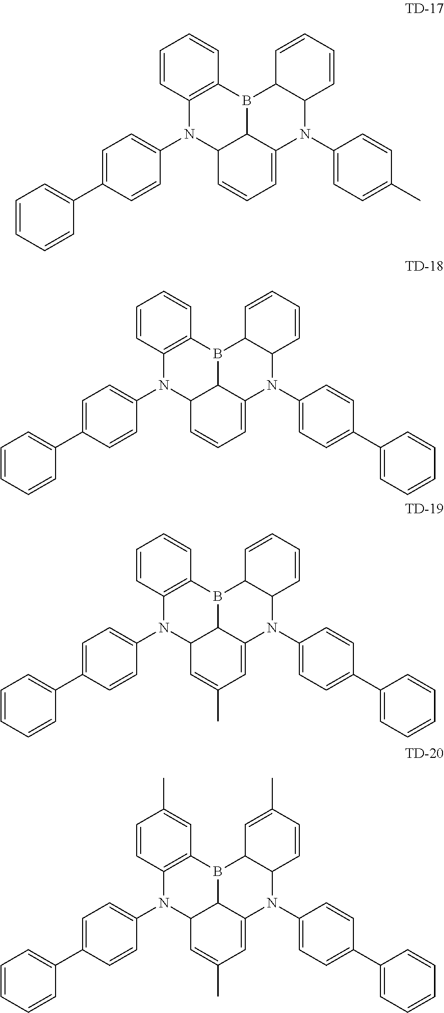 [SMILES: Cc1ccc(N2C3=CC=CC4C3B(c3ccccc3N4c3ccc(-c4ccccc4)cc3)C3C=CC=CC32)cc1 ; C1=CC2B3c4ccccc4N(c4ccc(-c5ccccc5)cc4)C4C=CC=C(C34)N(c3ccc(-c4ccccc4)cc3)C2C=C1 ; CC1=CC2C3B(c4ccccc4N2c2ccc(-c4ccccc4)cc2)C2C=CC=CC2N(c2ccc(-c4ccccc4)cc2)C3=C1 ; CC1=CC2B3c4cc(C)ccc4N(c4ccc(-c5ccccc5)cc4)C4C=C(C)C=C(C34)N(c3ccc(-c4ccccc4)cc3)C2C=C1]

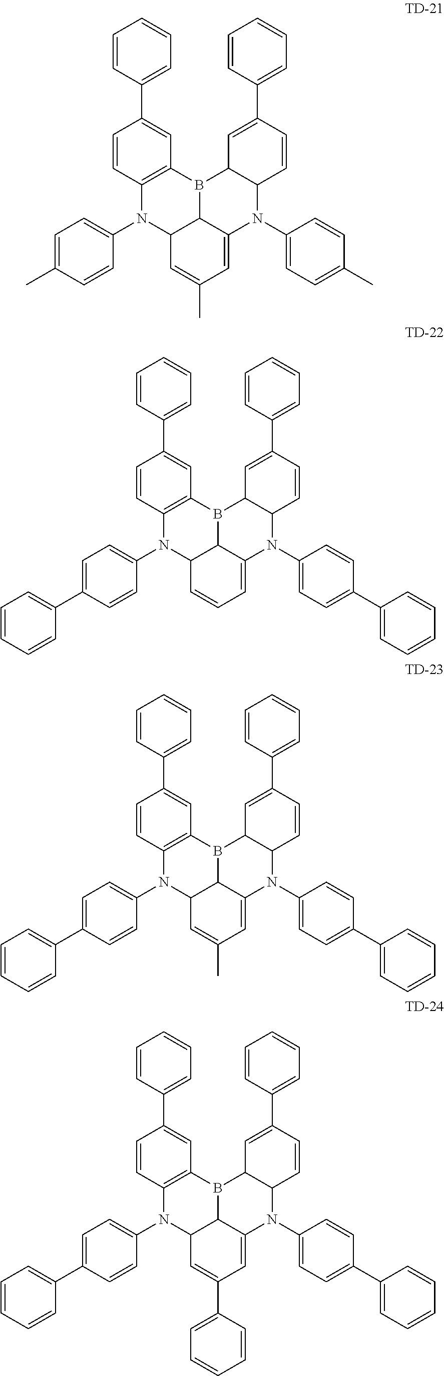

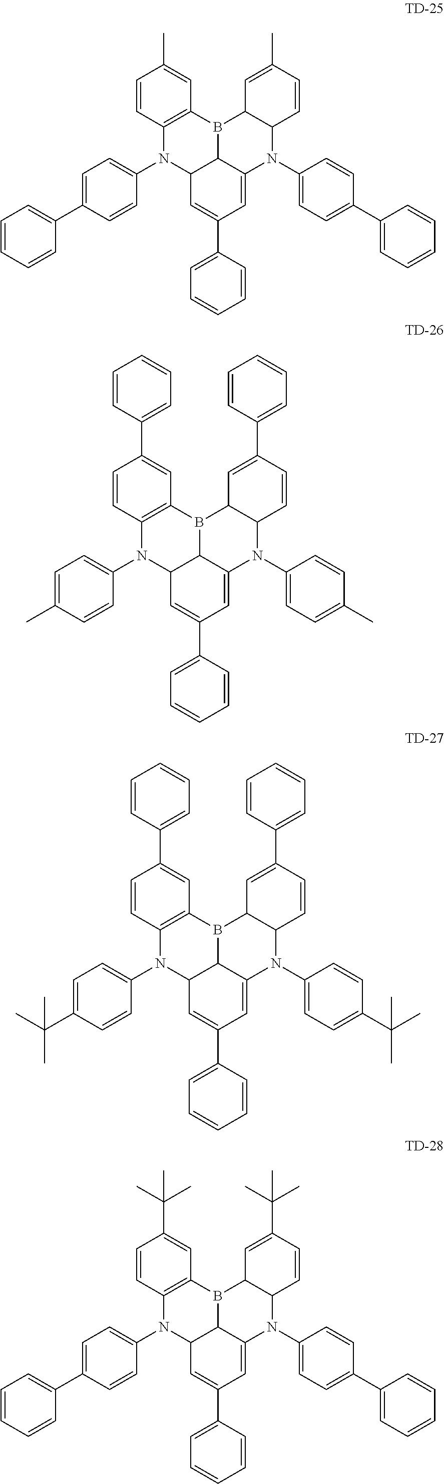

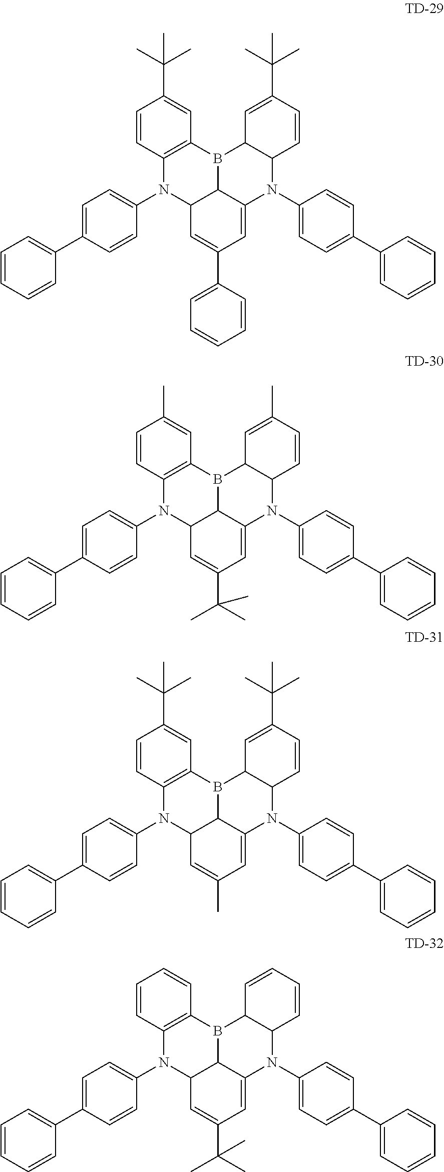 [SMILES: CC(C)(C)C1=CC2B3c4cc(C(C)(C)C)ccc4N(c4ccc(-c5ccccc5)cc4)C4C=C(c5ccccc5)C=C(C34)N(c3ccc(-c4ccccc4)cc3)C2C=C1 ; CC1=CC2B3c4cc(C)ccc4N(c4ccc(-c5ccccc5)cc4)C4C=C(C(C)(C)C)C=C(C34)N(c3ccc(-c4ccccc4)cc3)C2C=C1 ; CC1=CC2C3B(c4cc(C(C)(C)C)ccc4N2c2ccc(-c4ccccc4)cc2)C2C=C(C(C)(C)C)C=CC2N(c2ccc(-c4ccccc4)cc2)C3=C1 ; CC(C)(C)C1=CC2C3B(c4ccccc4N2c2ccc(-c4ccccc4)cc2)C2C=CC=CC2N(c2ccc(-c4ccccc4)cc2)C3=C1]

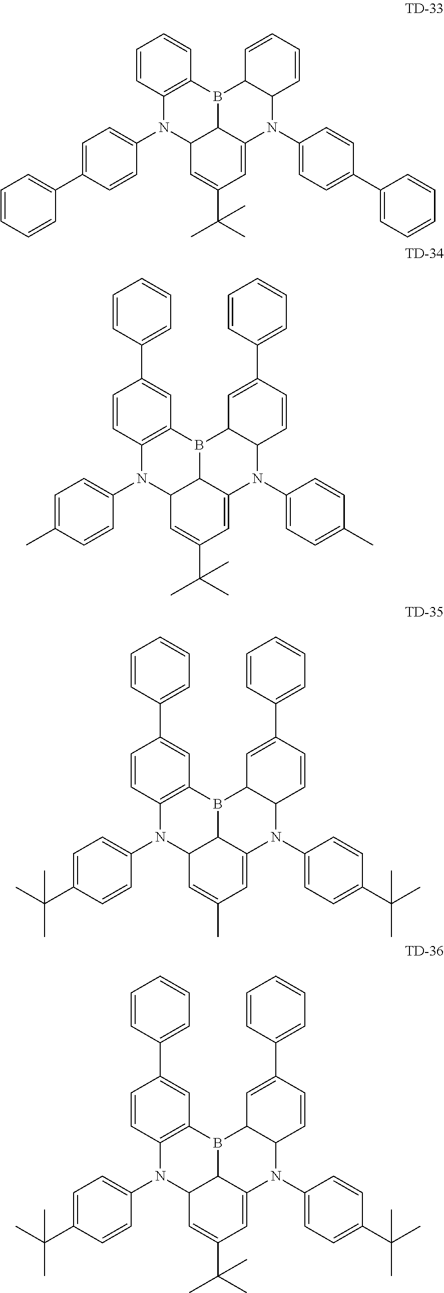

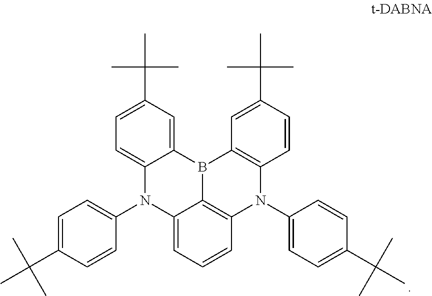 [SMILES: CC(C)(C)c1ccc(N2c3ccc(C(C)(C)C)cc3B3c4cc(C(C)(C)C)ccc4N(c4ccc(C(C)(C)C)cc4)c4cccc2c43)cc1]

### Quantum Dot

The electronic apparatus may include quantum dots. For example, the electronic apparatus may include a color filter, and the color filter may include quantum dots.

The term “quantum dot” as used herein refers to a crystal of a semiconductor compound, and may include any material capable of emitting light of various suitable emission wavelengths according to the size of the crystal. Quantum dots may emit light of various suitable emission wavelengths by adjusting the element ratio in the quantum dot compound.

A diameter of the quantum dot may be, for example, in a range of about 1 nm to about 10 nm.

The quantum dot may be synthesized by a wet chemical process, a metal organic chemical vapor deposition process, a molecular beam epitaxy process, and/or any process similar thereto.

The wet chemical process is a method including mixing a precursor material with an organic solvent and then growing a quantum dot particle crystal. When the crystal grows, the organic solvent naturally acts as a dispersant coordinated on the surface of the quantum dot crystal and controls the growth of the crystal so that the growth of quantum dot particles can be controlled through a process which costs lower, and is easier than vapor deposition methods, such as metal organic chemical vapor deposition (MOCVD) or molecular beam epitaxy (MBE).

The quantum dot may include Group II-VI semiconductor compounds, Group III-V semiconductor compounds, Group III-VI semiconductor compounds, Group I-III-VI semiconductor compounds, Group IV-VI semiconductor compounds, a Group IV element or compound, or a combination thereof.

Examples of the Group II-VI semiconductor compound are a binary compound, such as CdS, CdSe, CdTe, ZnS, ZnSe, ZnTe, ZnO, HgS, HgSe, HgTe, MgSe, and/or MgS; a ternary compound, such as CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, and/or MgZnS; a quaternary compound, such as CdZnSeS, CdZnSeTe, CdZnSTe, CdHgSeS, CdHgSeTe, CdHgSTe, HgZnSeS, HgZnSeTe, and/or HgZnSTe; or a combination thereof.

Examples of the Group III-V semiconductor compound are: a binary compound, such as GaN, GaP, GaAs, GaSb, AlN, AlP, AlAs, AlSb, InN, InP, InAs, InSb, and the like; a ternary compound, such as GaNP, GaNAs, GaNSb, GaPAs, GaPSb, AlNP, AlNAs, AlNSb, AlPAs, AlPSb, InGaP, InNP, InAlP, InNAs, InNSb, InPAs, InPSb, and the like; a quaternary compound, such as GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, InAlPSb, and the like; or any combination thereof. In embodiments, the Group III-V semiconductor compound may further include a Group II element. Examples of the Group III-V semiconductor compound further including a Group II element are InZnP, InGaZnP, InAlZnP, and/or the like.

Examples of the Group III-VI semiconductor compound are: a binary compound, such as GaS, GaSe, Ga₂Se₃, GaTe, InS, InSe, In₂Se₃, and/or InTe; a ternary compound, such as InGaS₃ and/or InGaSes; or any combination thereof.

Examples of the Group I-III-VI semiconductor compound are: a ternary compound, such as AgInS, AgInS₂, CuInS, CuInS₂, CuGaO₂, AgGaO₂, AgAlO₂, and/or the like; a quaternary compound, such as AgInGaS, AgInGaS₂, and/or the like; or any combination thereof.

Examples of the Group IV-VI semiconductor compound are: a binary compound, such as SnS, SnSe, SnTe, PbS, PbSe, and/or PbTe; a ternary compound, such as SnSeS, SnSeTe, SnSTe, PbSeS, PbSeTe, PbSTe, SnPbS, SnPbSe, and/or SnPbTe; a quaternary compound, such as SnPbSSe, SnPbSeTe, and/or SnPbSTe; or a combination thereof.

The Group IV element or compound may include: a single element, such as Si and/or Ge; a binary compound, such as SiC and/or SiGe; or a combination thereof.

Each element included in a multi-element compound such as the binary compound, the ternary compound, and the quaternary compound may be present at a uniform concentration or non-uniform concentration in a particle.

In embodiments, the quantum dot may have a single structure in which the concentration of each element in the quantum dot is uniform, or a core-shell dual structure. For example, the material included in the core and the material included in the shell may be different from each other.

The shell of the quantum dot may act as a protective layer that prevents or reduces chemical degeneration of the core to maintain semiconductor characteristics, and/or as a charging layer that imparts electrophoretic characteristics to the quantum dot. The shell may be a single layer or a multi-layer. The interface between the core and the shell may have a concentration gradient in which the concentration of elements present in the shell decreases along a direction toward the center of the core.

Examples of the shell of the quantum dot may be an oxide of metal, metalloid, and/or non-metal, a semiconductor compound, and a combination thereof. Examples of the oxide of metal, metalloid, and/or non-metal are a binary compound, such as SiO₂, Al₂O₃, TiO₂, ZnO, MnO, Mn₂O₃, Mn₃O₄, CuO, FeO, Fe₂O₃, Fe₃O₄, CoO, Co₃O₄, and/or NiO; a ternary compound, such as MgAl₂O₄, CoFe₂O₄, NiFe₂O₄, and/or CoMn₂O₄; and a combination thereof. Examples of the semiconductor compound are, as described herein, a Group II-VI semiconductor compound; a Group III-V semiconductor compound; a Group III-VI semiconductor compound; a Group I-III-VI semiconductor compound; a Group IV-VI semiconductor compound; and a combination thereof. For example, the semiconductor compound may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, or a combination thereof.

A full width at half maximum (FWHM) of the emission wavelength spectrum of the quantum dot may be about 45 nm or less, for example, about 40 nm or less, for example, about 30 nm or less, and within these ranges, color purity and/or color reproducibility may be increased. In embodiments, light emitted through these quantum dots is emitted in all (e.g., substantially all) directions, which may improve the viewing angle.

In embodiments, the quantum dot may be in the form of a spherical particle (e.g., a substantially spherical particle), a pyramidal particle (e.g., a substantially pyramidal particle), a multi-arm particle, a cubic nanoparticle (e.g., a substantially cubic nanoparticle), a nanotube particle, a nanowire particle, a nanofiber particle, and/or a nanoplate particle.

Because an energy band gap may be adjusted by controlling the size of the quantum dot, light having various suitable wavelength bands may be obtained from the quantum dot emission layer. Accordingly, by using quantum dots of different sizes, a light-emitting device that emits light of various suitable wavelengths may be implemented. In one or more embodiments, the size of the quantum dot may be selected to emit red, green and/or blue light. In embodiments, the size of the quantum dots may be configured such that light of various suitable colors is combined to emit white light.

### Electron Transport Region in Interlayer 130

The electron transport region may have: i) a single-layered structure consisting of a single layer consisting of a single material, ii) a single-layered structure consisting of a single layer including a plurality of different materials, or iii) a multilayer structure including a plurality of layers including a plurality of different materials.

The electron transport region may include: an electron transport layer; and a hole-blocking layer, an electron injection layer, or a combination thereof.

For example, the electron transport region may have a structure including an electron transport layer/electron injection layer structure or a hole blocking layer/electron transport layer/electron injection layer structure, wherein in each structure, constituting layers are sequentially stacked from the emission layer.

The electron transport region (for example, a hole blocking layer or an electron transport layer in the electron transport region) may include a metal-free compound including at least one π electron-deficient nitrogen-containing C₁-C₆₀ cyclic group.

According to an embodiment, the electron transport region may include a compound represented by Formula 601.

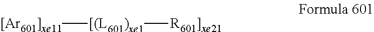

In Formula 601,
Ar₆₀₁ and L₆₀₁ may each independently be a C₃-C₆₀ carbocyclic group that is unsubstituted or substituted with at least one R₁₀ₐ or a C₁-C₆₀ heterocyclic group that is unsubstituted or substituted with at least one R₁₀ₐ,
xe11 may be 1, 2, or 3,
xe1 may be 0, 1, 2, 3, 4, or 5,
R₆₀₁ may be a C₃-C₆₀ carbocyclic group that is unsubstituted or substituted with at least one R₁₀ₐ, a C₁-C₆₀ heterocyclic group that is unsubstituted or substituted with at least one R₁₀ₐ, —Si(Q₆₀₁)(Q₆₀₂)(Q₆₀₃), —C(═O)(Q₆₀₁), —S(═O)₂(Q₆₀₁), or —P(═O)(Q₆₀₁)(Q₆₀₂),
Q₆₀₁ to Q₆₀₃ are each as described in connection with Q₁,
xe21 may be 1, 2, 3, 4, or 5, and
at least one selected from among Ar₆₀₁, L₆₀₁, and R₆₀₁ may each independently be a π electron-deficient nitrogen-containing C₁-C₆₀ cyclic group that is unsubstituted or substituted with at least one R₁₀ₐ.

According to an embodiment, when xe11 in Formula 601 is 2 or more, two or more of Ar₆₀₁ may be linked together via a single bond (e.g., a covalent single bond).

According to an embodiment, Ar₆₀₁ in Formula 601 may be a substituted or unsubstituted anthracene group.

According to an embodiment, the electron transport region may include a compound represented by Formula 601-1:

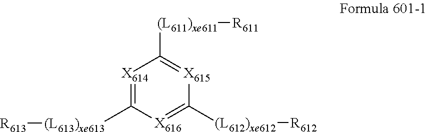

wherein, in Formula 601-1,
X₆₁₄ may be N or C(R₆₁₄), X₆₁₅ may be N or C(R₆₁₅), X₆₁₆ may be N or C(R₆₁₆), and at least one selected from among X₆₁₄ to X₆₁₆ may be N,
L₆₁₁ to L₆₁₃ are each as described in connection with L₆₀₁,
xe611 to xe613 are each as described in connection with xe1,
R₆₁₁ to R₆₁₃ are each as described in connection with R₆₀₁, and
R₆₁₄ to R₆₁₆ may each independently be hydrogen, deuterium, —F, —Cl, —Br, —I, a hydroxyl group, a cyano group, a nitro group, a C₁-C₂₀ alkyl group, a C₁-C₂₀ alkoxy group, a C₃-C₆₀ carbocyclic group that is unsubstituted or substituted with at least one R₁₀ₐ, or a C₁-C₆₀ heterocyclic group that is unsubstituted or substituted with at least one R₁₀ₐ.

According to an embodiment, xe1 and xe611 to xe613 in Formulae 601 and 601-1 may each independently be 0, 1, or 2.

The electron transport region may include one selected from among Compounds ET1 to ET45, 2,9-dimethyl-4,7-diphenyl-1,10-phenanthroline (BCP), 4,7-diphenyl-1,10-phenanthroline (Bphen), Alq₃, BAlq, TAZ, NTAZ, or any combination thereof:

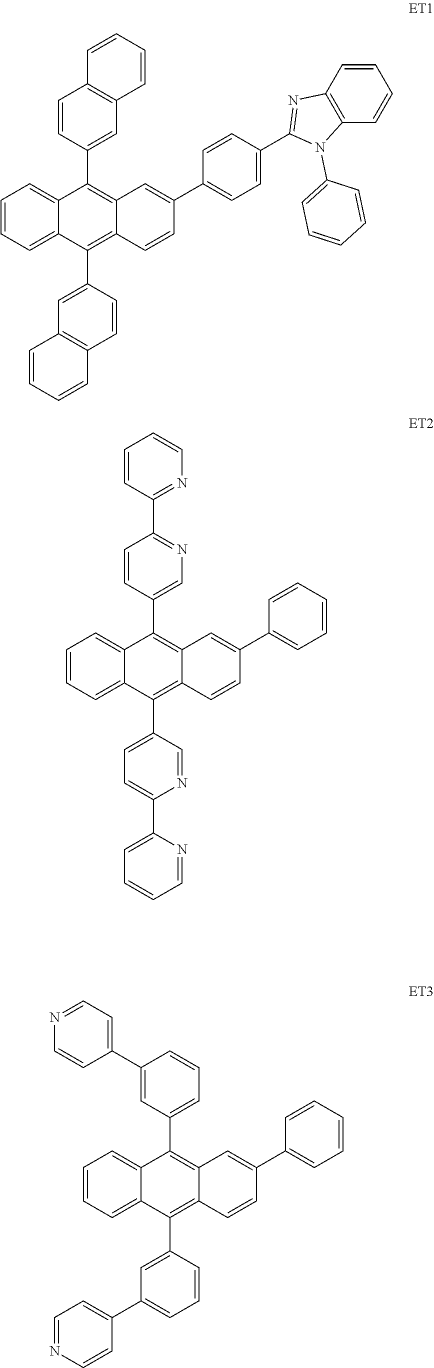 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

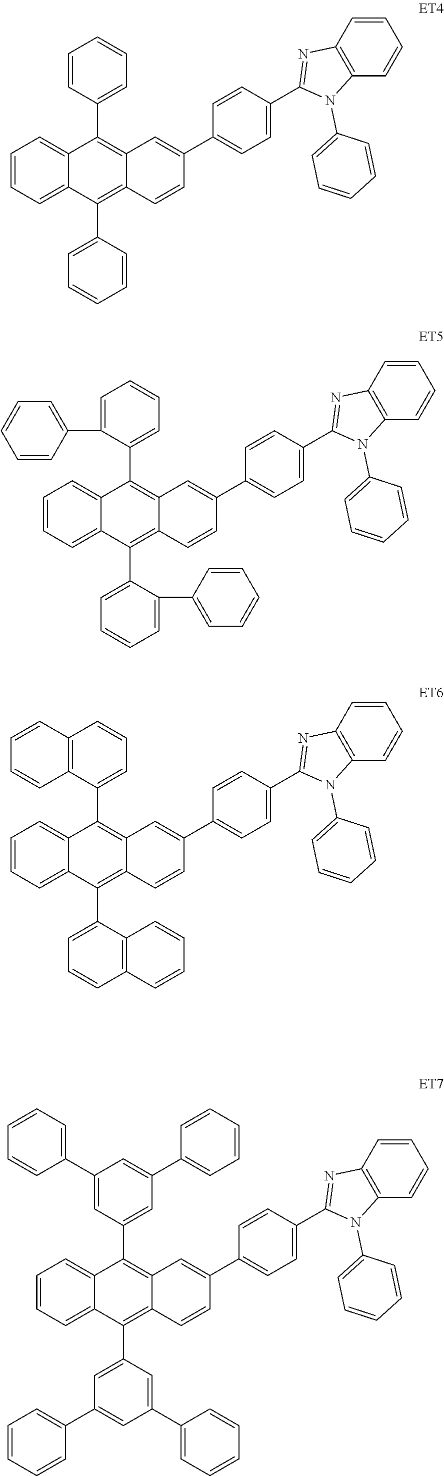 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1 ; c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1]

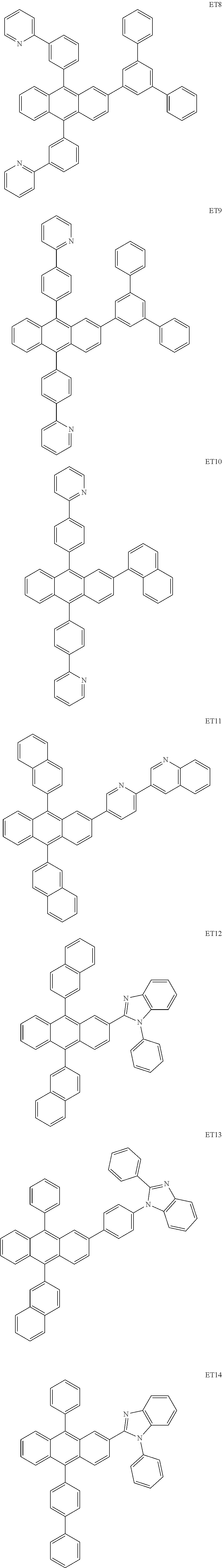

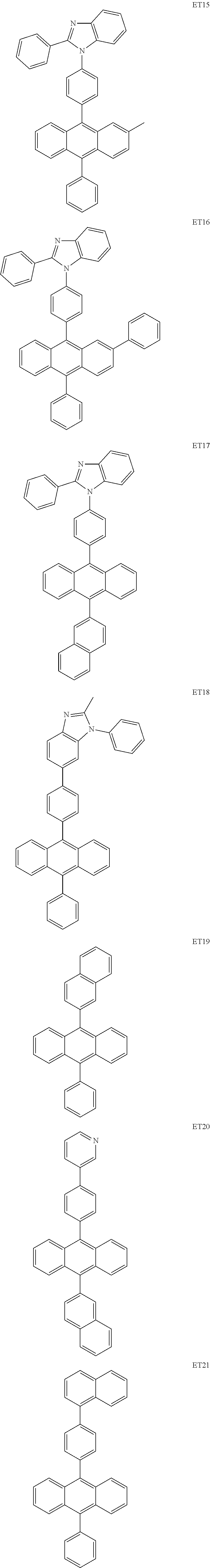

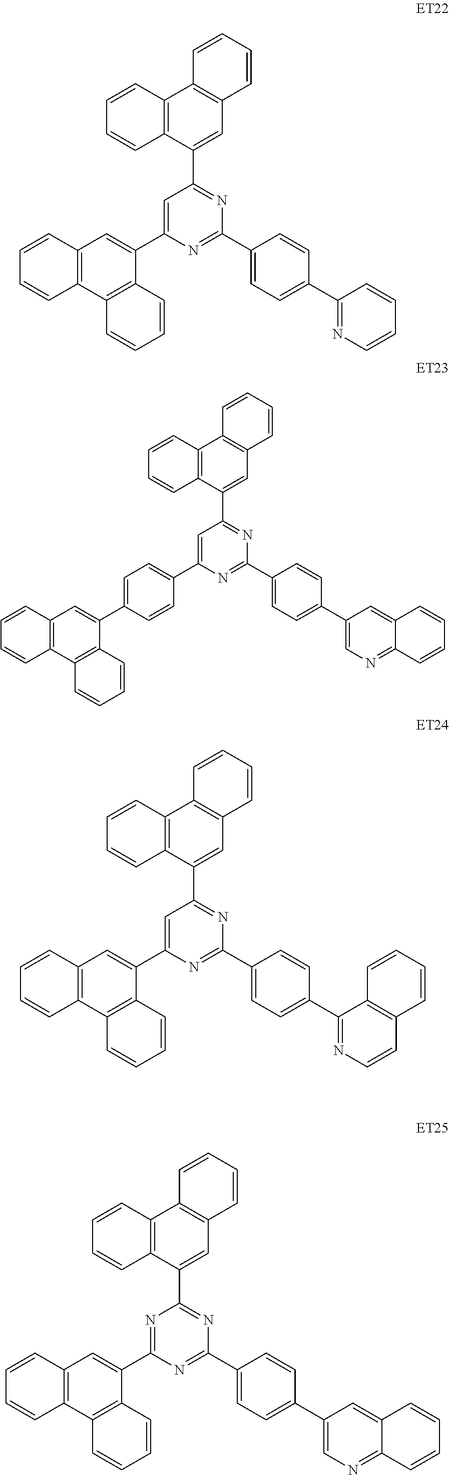 [SMILES: c1ccc(-c2ccc(-c3nc(-c4cc5ccccc5c5ccccc45)cc(-c4cc5ccccc5c5ccccc45)n3)cc2)nc1 ; c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1 ; c1ccc2ncc(-c3ccc(-c4nc(-c5cc6ccccc6c6ccccc56)nc(-c5cc6ccccc6c6ccccc56)n4)cc3)cc2c1]

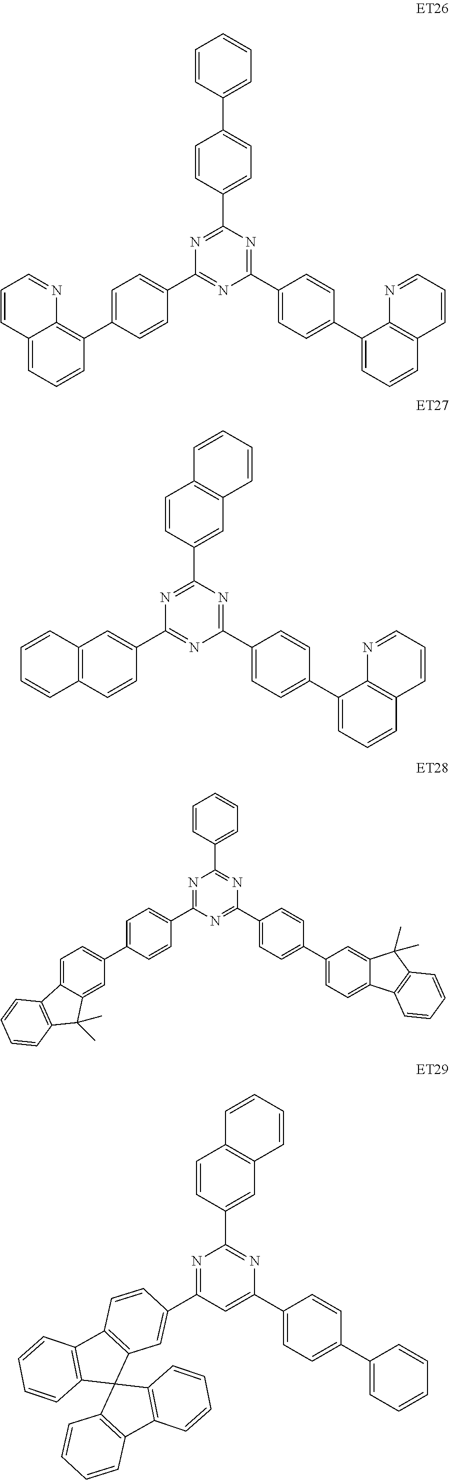 [SMILES: c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1 ; CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2ccc(-c3cc(-c4ccc5c(c4)C4(c6ccccc6-c6ccccc64)c4ccccc4-5)nc(-c4ccc5ccccc5c4)n3)cc2)cc1]

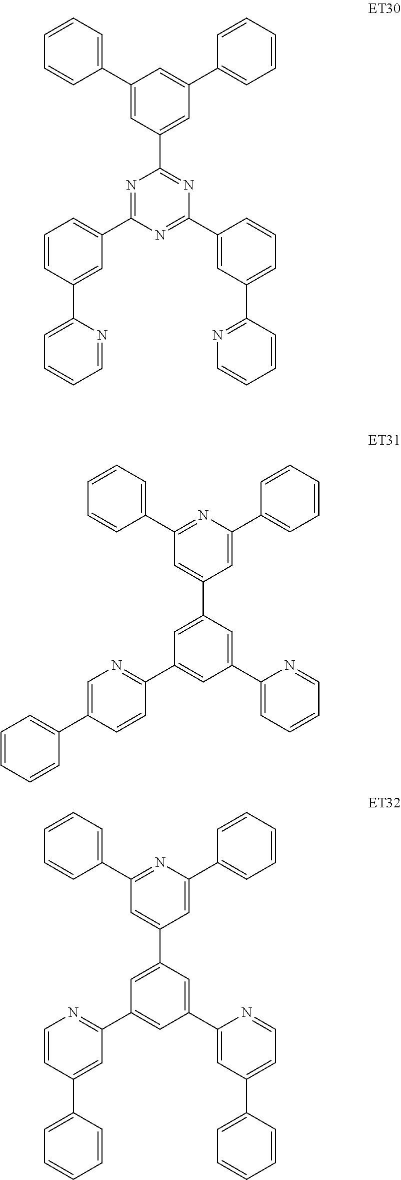 [SMILES: c1ccc(-c2cc(-c3ccccc3)cc(-c3nc(-c4cccc(-c5ccccn5)c4)nc(-c4cccc(-c5ccccn5)c4)n3)c2)cc1 ; c1ccc(-c2ccc(-c3cc(-c4cc(-c5ccccc5)nc(-c5ccccc5)c4)cc(-c4ccccn4)c3)nc2)cc1 ; c1ccc(-c2ccnc(-c3cc(-c4cc(-c5ccccc5)nc(-c5ccccc5)c4)cc(-c4cc(-c5ccccc5)ccn4)c3)c2)cc1]

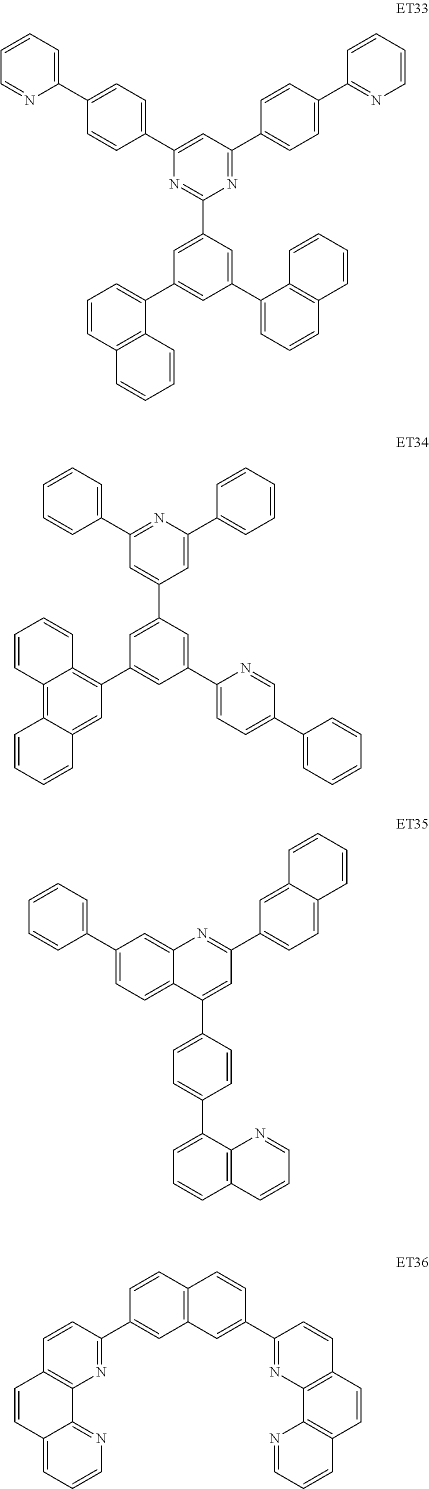 [SMILES: c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1 ; c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12]

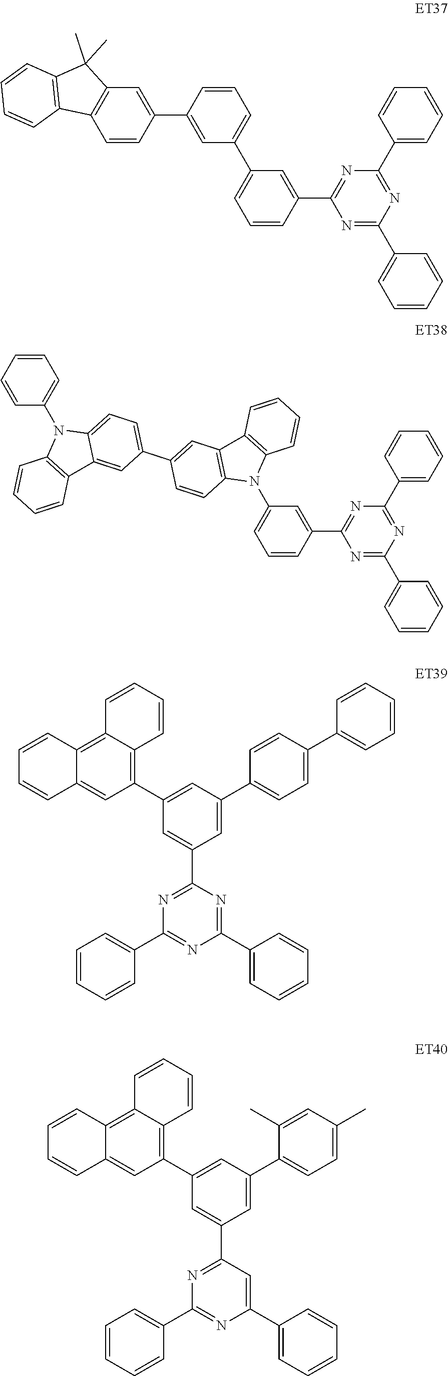 [SMILES: CC1(C)c2ccccc2-c2ccc(-c3cccc(-c4cccc(-c5nc(-c6ccccc6)nc(-c6ccccc6)n5)c4)c3)cc21 ; c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1 ; c1ccc(-c2ccc(-c3cc(-c4nc(-c5ccccc5)nc(-c5ccccc5)n4)cc(-c4cc5ccccc5c5ccccc45)c3)cc2)cc1 ; Cc1ccc(-c2cc(-c3cc(-c4ccccc4)nc(-c4ccccc4)n3)cc(-c3cc4ccccc4c4ccccc34)c2)c(C)c1]

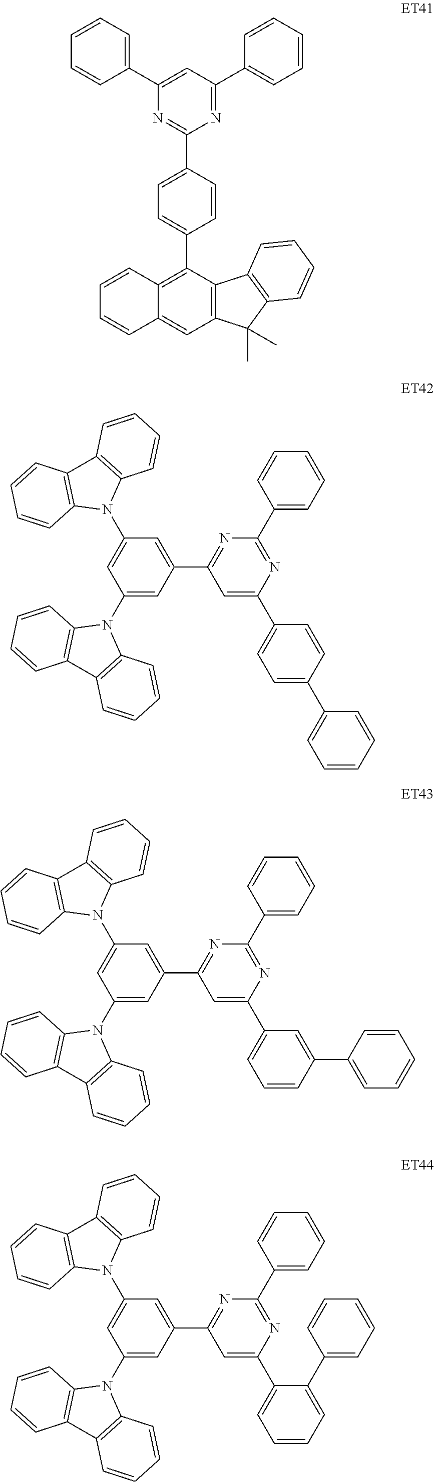 [SMILES: CC1(C)c2ccccc2-c2c1cc1ccccc1c2-c1ccc(-c2nc(-c3ccccc3)cc(-c3ccccc3)n2)cc1 ; c1ccc(-c2ccc(-c3cc(-c4cc(-n5c6ccccc6c6ccccc65)cc(-n5c6ccccc6c6ccccc65)c4)nc(-c4ccccc4)n3)cc2)cc1 ; c1ccc(-c2cccc(-c3cc(-c4cc(-n5c6ccccc6c6ccccc65)cc(-n5c6ccccc6c6ccccc65)c4)nc(-c4ccccc4)n3)c2)cc1 ; c1ccc(-c2nc(-c3cc(-n4c5ccccc5c5ccccc54)cc(-n4c5ccccc5c5ccccc54)c3)cc(-c3ccccc3-c3ccccc3)n2)cc1]

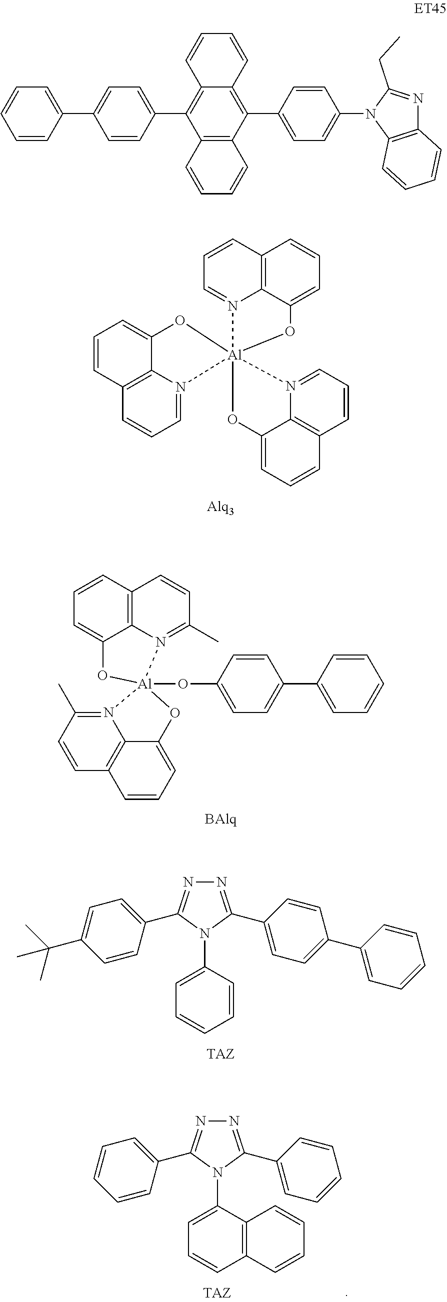

The thickness of the electron transport region may be about 100 Å to about 5,000 Å, for example, about 160 Å to about 4,000 Å. When the electron transport region includes the hole blocking layer, the electron transport layer, or any combination thereof, a thickness of the hole blocking layer or electron transport layer may each independently be from about 20 Å to about 1,000 Å, for example, about 30 Å to about 300 Å, and the thickness of the electron transport layer may be from about 100 Å to about 1,000 Å, for example, about 150 Å to about 500 Å. When the thicknesses of the hole blocking layer and/or the electron transport layer are within these ranges as described above, suitable or satisfactory electron-transporting characteristics may be obtained without a substantial increase in driving voltage.

The electron transport region (for example, the electron transport layer in the electron transport region) may further include, in addition to the materials described above, a metal-containing material.

The metal-containing material may include an alkali metal complex, an alkaline earth metal complex, or any combination thereof. A metal ion of the alkali metal complex may be a Li ion, a Na ion, a K ion, a Rb ion, or a Cs ion, and a metal ion of the alkaline earth metal complex may be a Be ion, a Mg ion, a Ca ion, a Sr ion, or a Ba ion. A ligand coordinated with the metal ion of the alkali metal complex or the alkaline earth-metal complex may include a hydroxyquinoline, a hydroxyisoquinoline, a hydroxybenzoquinoline, a hydroxyacridine, a hydroxyphenanthridine, a hydroxyphenyloxazole, a hydroxyphenylthiazole, a hydroxyphenyloxadiazole, a hydroxyphenylthiadiazole, a hydroxyphenylpyridine, a hydroxyphenylbenzimidazole, a hydroxyphenylbenzothiazole, a bipyridine, a phenanthroline, a cyclopentadiene, or any combination thereof.

According to an embodiment, the metal-containing material may include a Li complex. The Li complex may include, for example, Compound ET-D1 (LiQ) and/or ET-D2:

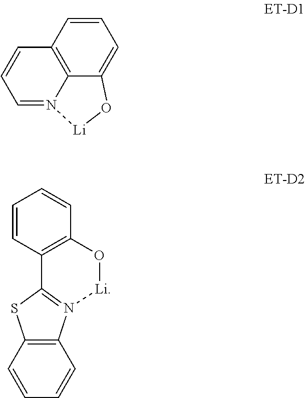

The electron transport region may include an electron injection layer that facilitates the injection of electrons from the second electrode 150. The electron injection layer may directly contact (e.g., physically contact) the second electrode 150.

The electron injection layer may have: i) a single-layered structure consisting of a single layer consisting of a single material, ii) a single-layered structure consisting of a single layer including a plurality of different materials, or iii) a multilayer structure including a plurality of layers including a plurality of different materials.

The electron injection layer may include an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof.

The alkali metal may include Li, Na, K, Rb, Cs, or any combination thereof. The alkaline earth metal may include Mg, Ca, Sr, Ba, or any combination thereof. The rare earth metal may include Sc, Y, Ce, Tb, Yb, Gd, or any combination thereof.

The alkali metal-containing compound, the alkaline earth metal-containing compound, and the rare earth metal-containing compound may include oxides, halides (for example, fluorides, chlorides, bromides, iodides, and/or the like), and/or tellurides of the alkali metal, the alkaline earth metal, and the rare earth metal, or any combination thereof.

The alkali metal-containing compound may include: alkali metal oxides, such as Li₂O, Cs₂O, and/or K₂O; alkali metal halides, such as LiF, NaF, CsF, KF, LiI, NaI, CsI, and/or KI; or any combination thereof. The alkaline earth metal-containing compound may include an alkaline earth metal compound, such as BaO, SrO, CaO, BaₓSr₁₋ₓO (x is a real number satisfying 0<x<1), and/or BaₓCa₁₋ₓO (x is a real number satisfying 0<x<1). The rare earth metal-containing compound may include YbF₃, ScF₃, Sc₂O₃, Y₂O₃, Ce₂O₃, GdF₃, TbF₃, YbI₃, ScI₃, TbI₃, or any combination thereof. According to an embodiment, the rare earth metal-containing compound may include lanthanide metal telluride. Examples of the lanthanide metal telluride may include LaTe, CeTe, PrTe, NdTe, PmTe, SmTe, EuTe, GdTe, TbTe, DyTe, HoTe, ErTe, TmTe, YbTe, LuTe, La₂Te₃, Ce₂Te₃, Pr₂Te₃, Nd₂Te₃, Pm₂Te₃, Sm₂Te₃, Eu₂Te₃, Gd₂Te₃, Tb₂Te₃, Dy₂Te₃, Ho₂Te₃, Er₂Te₃, Tm₂Te₃, Yb₂Te₃, and Lu₂Te₃.

The alkali metal complex, the alkaline earth-metal complex, and the rare earth metal complex may include i) one of ions of the alkali metal, the alkaline earth metal, and the rare earth metal and ii) a ligand bonded to the metal ion, for example, hydroxyquinoline, hydroxyisoquinoline, hydroxybenzoquinoline, hydroxyacridine, hydroxyphenanthridine, hydroxyphenyloxazole, hydroxyphenylthiazole, hydroxyphenyloxadiazole, hydroxyphenylthiadiazole, hydroxyphenylpyridine, hydroxyphenyl benzimidazole, hydroxyphenylbenzothiazole, bipyridine, phenanthroline, cyclopentadiene, or any combination thereof.

The electron injection layer may include or consist of an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof, as described above. According to an embodiment, the electron injection layer may further include an organic material (for example, a compound represented by Formula 601).

According to an embodiment, the electron injection layer may include or consist of i) an alkali metal-containing compound (for example, alkali metal halide), ii) a) an alkali metal-containing compound (for example, alkali metal halide); and b) an alkali metal, an alkaline earth metal, a rare earth metal, or any combination thereof. According to an embodiment, the electron injection layer may be a KI:Yb co-deposited layer, an RbI:Yb co-deposited layer, a LiF:Yb co-deposited layer, and/or the like.

When the electron injection layer further includes an organic material, the alkali metal, the alkaline earth metal, the rare earth metal, the alkali metal-containing compound, the alkaline earth metal-containing compound, the rare earth metal-containing compound, the alkali metal complex, the alkaline earth-metal complex, the rare earth metal complex, or any combination thereof may be uniformly or non-uniformly dispersed in a matrix including the organic material.

The thickness of the electron injection layer may be about 1 Å to about 100 Å, or, for example, about 3 Å to about 90 Å. When the thickness of the electron injection layer is within the ranges as described above, suitable or satisfactory electron injection characteristics may be obtained without a substantial increase in driving voltage.

### Second Electrode 150

The second electrode 150 is on the interlayer 130. The second electrode 150 may be a cathode, which is an electron injection electrode, and as a material for forming the second electrode 150, a metal, an alloy, an electrically conductive compound, or any combination thereof, each having a low-work function, may be used.

The second electrode 150 may include lithium (Li), silver (Ag), magnesium (Mg), aluminum (AI), aluminum-lithium (Al—Li), calcium (Ca), magnesium-indium (Mg—In), magnesium-silver (Mg—Ag), ytterbium (Yb), silver-ytterbium (Ag—Yb), ITO, IZO, or any combination thereof. The second electrode 150 may be a transmissive electrode, a transflective electrode, or a reflective electrode.

The second electrode 150 may have a single-layer structure or a multilayer structure including a plurality of layers.

### Capping Layers

A first capping layer may be outside the first electrode 110, and/or a second capping layer may be outside the second electrode 150. In embodiments, the light-emitting device 10 may have a structure in which the first capping layer, the first electrode 110, the interlayer 130, and the second electrode 150 are sequentially stacked in the stated order, a structure in which the first electrode 110, the interlayer 130, the second electrode 150, and the second capping layer are sequentially stacked in the stated order, or a structure in which the first capping layer, the first electrode 110, the interlayer 130, the second electrode 150, and the second capping layer are sequentially stacked in the stated order.

Light generated in the emission layer of the interlayer 130 of the light-emitting device 10 may be extracted toward the outside through the first electrode 110 which is a transflective electrode or a transmissive electrode, and the first capping layer. Light generated in the emission layer of the interlayer 130 of the light-emitting device 10 may be extracted toward the outside through the second electrode 150 which is a transflective electrode or a transmissive electrode, and the second capping layer.

The first capping layer and the second capping layer may increase external emission efficiency according to the principle of constructive interference. Accordingly, the light extraction efficiency of the light-emitting device 10 is increased, such that the luminescence efficiency of the light-emitting device 10 may be increased.

Each of the first capping layer and the second capping layer may include a material having a refractive index of 1.6 or more (at a wavelength of 589 nm).

The first capping layer and the second capping layer may each independently be an organic capping layer including an organic material, an inorganic capping layer including an inorganic material, or an organic-inorganic composite capping layer including an organic material and an inorganic material.

At least one selected from among the first capping layer and the second capping layer may each independently include a carbocyclic compound, a heterocyclic compound, an amine group-containing compound, a porphine derivative, a phthalocyanine derivative, a naphthalocyanine derivative, an alkali metal complex, an alkaline earth metal complex, or any combination thereof. The carbocyclic compound, the heterocyclic compound, and the amine group-containing compound may optionally be substituted with a substituent including O, N, S, Se, Si, F, Cl, Br, I, or any combination thereof. According to an embodiment, at least one selected from among the first capping layer and the second capping layer may each independently include an amine group-containing compound.

According to an embodiment, at least one selected from among the first capping layer and the second capping layer may each independently include a compound represented by Formula 201, a compound represented by Formula 202, or any combination thereof.

According to an embodiment, at least one selected from among the first capping layer and the second capping layer may each independently include one selected from among Compounds HT28 to HT33, one selected from among Compounds CP1 to CP6, β-NPB, or any combination thereof:

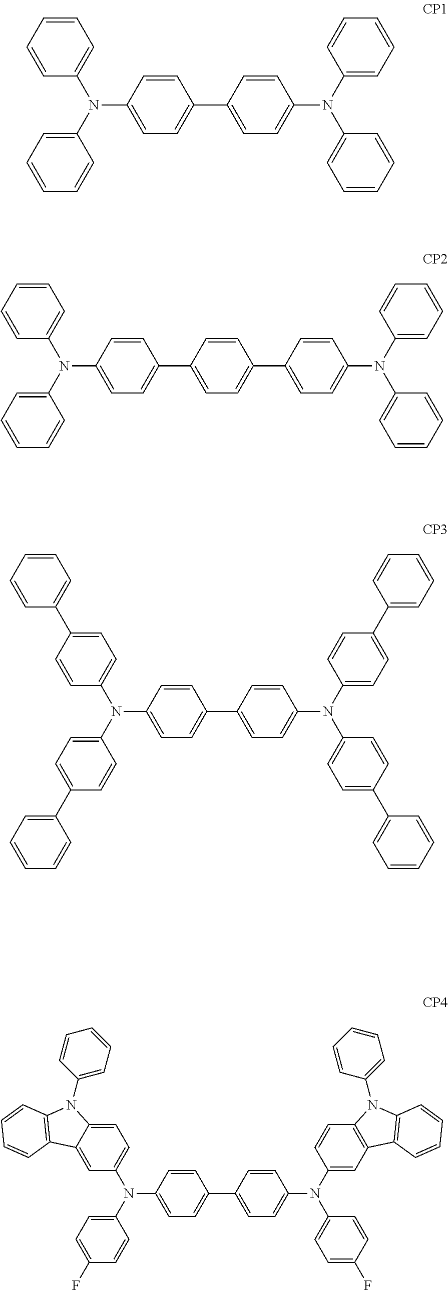 [SMILES: c1ccc(N(c2ccccc2)c2ccc(-c3ccc(N(c4ccccc4)c4ccccc4)cc3)cc2)cc1 ; c1ccc(N(c2ccccc2)c2ccc(-c3ccc(-c4ccc(N(c5ccccc5)c5ccccc5)cc4)cc3)cc2)cc1 ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; Fc1ccc(N(c2ccc(-c3ccc(N(c4ccc(F)cc4)c4ccc5c(c4)c4ccccc4n5-c4ccccc4)cc3)cc2)c2ccc3c(c2)c2ccccc2n3-c2ccccc2)cc1]

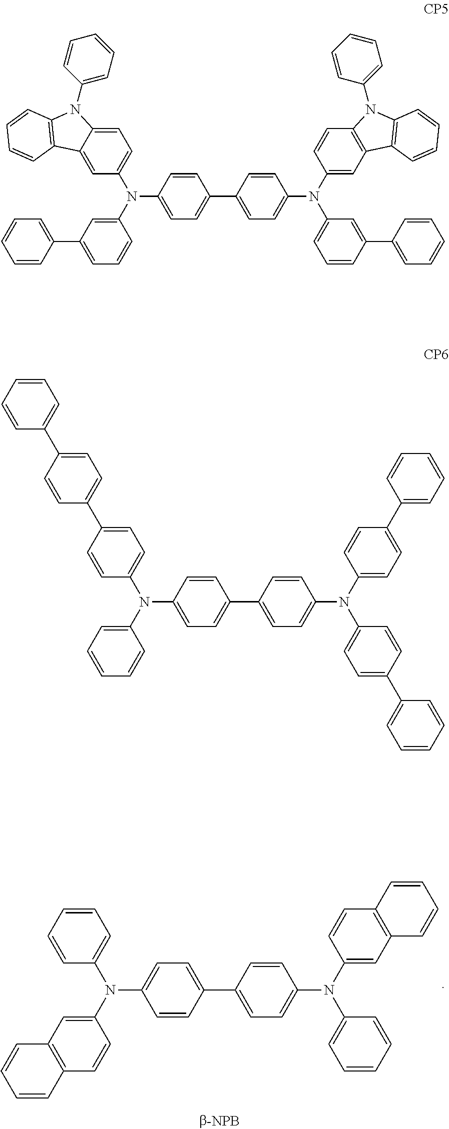 [SMILES: c1ccc(-c2cccc(N(c3ccc(-c4ccc(N(c5cccc(-c6ccccc6)c5)c5ccc6c(c5)c5ccccc5n6-c5ccccc5)cc4)cc3)c3ccc4c(c3)c3ccccc3n4-c3ccccc3)c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1]

### Electronic Apparatus

The light-emitting device may be included in various suitable electronic apparatuses. For example, the electronic apparatus including the light-emitting device may be a light-emitting apparatus, an authentication apparatus, and/or the like.

The electronic apparatus (for example, a light-emitting apparatus) may further include, in addition to the light-emitting device, i) a color filter, ii) a color conversion layer, or iii) a color filter and a color conversion layer. The color filter and/or the color conversion layer may be provided in at least one traveling direction of light emitted from the light-emitting device. For example, the light emitted from the light-emitting device may be blue light. A detailed description of the light-emitting device is provided above. According to an embodiment, the color conversion layer may include quantum dots. The quantum dot may be, for example, a quantum dot as described herein.

The electronic apparatus may include a first substrate. The first substrate may include a plurality of subpixel areas, the color filter may include a plurality of color filter areas respectively corresponding to the subpixel areas, and the color conversion layer may include a plurality of color conversion areas respectively corresponding to the subpixel areas.

A pixel-defining film is between the plurality of subpixel areas, and each subpixel area is defined.

The color filter may further include a plurality of color filter areas and light-shielding patterns provided among the color filter areas, and the color conversion layer may further include a plurality of color conversion areas and light-shielding patterns provided among the color conversion areas.

The plurality of color filter areas (or the plurality of color conversion areas) may include a first area to emit a first color light, a second area to emit a second color light, and/or a third area to emit a third color light, wherein the first color light, the second color light, and/or the third color light may have different maximum emission wavelengths. According to an embodiment, the first color light may be red light, the second color light may be green light, and the third color light may be blue light. According to an embodiment, the plurality of color filter areas (or the plurality of color conversion areas) may include quantum dots. In more detail, the first area may include red quantum dots, the second area may include green quantum dots, and the third area may not include quantum dots. A detailed description of the quantum dots is provided herein. The first area, the second area, and/or the third area may each further include a scatterer (e.g., a light scatterer).

According to an embodiment, the light-emitting device may emit a first light, the first area may absorb the first light to emit a first-1 color light, the second area may absorb the first light to emit a second-1 color light, and the third area may absorb the first light to emit a third-1 color light. In embodiments, the first-1 color light, the second-1 color light, and the third-1 color light may have different maximum emission wavelengths. In more detail, the first light may be blue light, the first-1 color light may be red light, the second-1 color light may be green light, and the third-1 color light may be blue light.

The electronic apparatus may further include a thin-film transistor, in addition to the light-emitting device as described above. The thin-film transistor may include a source electrode, a drain electrode, and an active layer, wherein any one selected from among the source electrode and the drain electrode may be electrically connected to the other of the first electrode and the second electrode of the light-emitting device.

The thin-film transistor may further include a gate electrode, a gate insulating film, and/or the like.

The activation layer may include crystalline silicon, amorphous silicon, an organic semiconductor, an oxide semiconductor, and/or the like.

The electronic apparatus may further include a sealing portion to seal the light-emitting device. The sealing portion may be between the color filter and/or the color conversion layer and the light-emitting device. The sealing portion allows light from the light-emitting device to be extracted to the outside, and concurrently (e.g., simultaneously) prevents or reduces penetration of ambient air and moisture into the light-emitting device. The sealing portion may be a sealing substrate including a transparent glass substrate and/or a plastic substrate. The sealing portion may be a thin-film encapsulation layer including at least one layer of an organic layer and/or an inorganic layer. When the sealing portion is a thin film encapsulation layer, the electronic apparatus may be flexible.

Various suitable functional layers may be additionally on the sealing portion, in addition to the color filter and/or the color conversion layer, according to the use of the electronic apparatus. Examples of the functional layers may include a touch screen layer and a polarizing layer. The touch screen layer may be a pressure-sensitive touch screen layer, a capacitive touch screen layer, and/or an infrared touch screen layer. The authentication apparatus may be, for example, a biometric authentication apparatus that authenticates an individual by using biometric information of a living body (for example, fingertips, pupils, and/or the like).

The authentication apparatus may further include, in addition to the light-emitting device as described above, a biometric information collector.

The electronic apparatus may be applied to various suitable displays, light sources, lighting, personal computers (for example, a mobile personal computer), mobile phones, digital cameras, electronic organizers, electronic dictionaries, electronic game machines, medical instruments (for example, electronic thermometers, sphygmomanometers, blood glucose meters, pulse measurement devices, pulse wave measurement devices, electrocardiogram displays, ultrasonic diagnostic devices, and/or endoscope displays), fish finders, various suitable measuring instruments, meters (for example, meters for a vehicle, an aircraft, and/or a vessel), projectors, and/or the like.

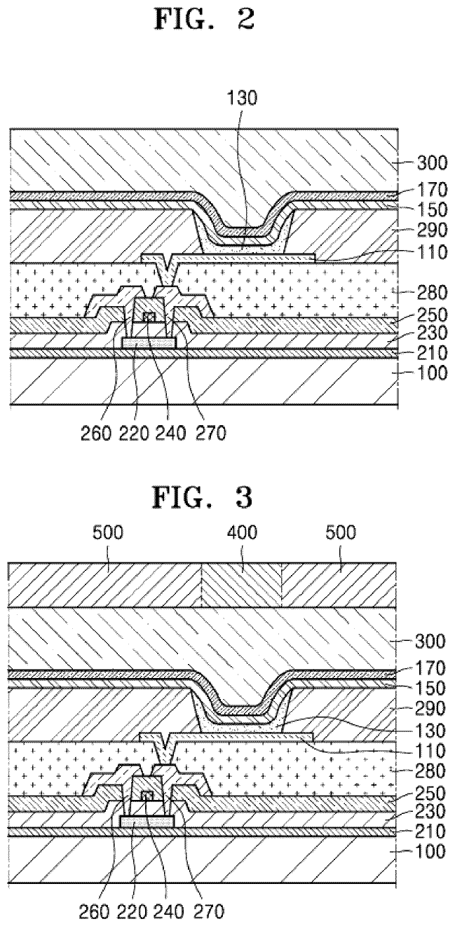

Description of FIGS. 2 and 3

FIG. 2 is a cross-sectional view of an electronic apparatus according to an embodiment.

The electronic apparatus of FIG. 2 includes a substrate 100, a thin-film transistor (TFT), a light-emitting device, and an encapsulation portion 300 that seals the light-emitting device.

The substrate 100 may be a flexible substrate, a glass substrate, and/or a metal substrate. A buffer layer 210 may be on the substrate 100. The buffer layer 210 may prevent or reduce penetration of impurities through the substrate 100 and may provide a flat surface on the substrate 100.

A TFT may be on the buffer layer 210. The TFT may include an activation layer 220, a gate electrode 240, a source electrode 260, and a drain electrode 270.

The activation layer 220 may include an inorganic semiconductor, such as silicon or polysilicon, an organic semiconductor, or an oxide semiconductor, and may include a source region, a drain region, and a channel region.

A gate insulating film 230 for insulating (e.g., electrically insulating) the activation layer 220 from the gate electrode 240 may be on the activation layer 220, and the gate electrode 240 may be on the gate insulating film 230.

An interlayer insulating film 250 may be on the gate electrode 240. The interlayer insulating film 250 may be between the gate electrode 240 and the source electrode 260 and between the gate electrode 240 and the drain electrode 270, to insulate (e.g., electrically insulate) these electrodes from one another.

The source electrode 260 and the drain electrode 270 may be on the interlayer insulating film 250. The interlayer insulating film 250 and the gate insulating film 230 may expose the source region and the drain region of the activation layer 220, and the source electrode 260 and the drain electrode 270 may be in contact (e.g., physical contact) with the exposed portions of the source region and the drain region of the activation layer 220.

The TFT may be electrically connected to a light-emitting device to drive the light-emitting device, and may be covered and protected by a passivation layer 280. The passivation layer 280 may include an inorganic insulating film (e.g., an inorganic electrically insulating film), an organic insulating film (e.g., an organic electrically insulating film), or any combination thereof. A light-emitting device may be provided on the passivation layer 280. The light-emitting device may include the first electrode 110, the interlayer 130, and the second electrode 150.

The first electrode 110 may be on the passivation layer 280. The passivation layer 280 may expose a portion of the drain electrode 270, not fully covering the drain electrode 270, and the first electrode 110 may be connected to the exposed portion of the drain electrode 270.

A pixel-defining film 290 including an insulating material (e.g., an electrically insulating material) may be on the first electrode 110. The pixel-defining film 290 may expose a certain region of the first electrode 110, and the interlayer 130 may be in the exposed region of the first electrode 110. The pixel-defining film 290 may be a polyimide-based organic film and/or a polyacrylic organic film. In embodiments, at least some layers of the interlayer 130 may extend beyond the upper portion of the pixel-defining film 290 to be provided in the form of a common layer.

The second electrode 150 may be on the interlayer 130, and a capping layer 170 may be additionally on the second electrode 150. The capping layer 170 may cover the second electrode 150.

The encapsulation portion 300 may be on the capping layer 170. The encapsulation portion 300 may be on a light-emitting device to protect the light-emitting device from moisture and/or oxygen. The encapsulation portion 300 may include: an inorganic film including silicon nitride (SiNx), silicon oxide (SiOx), indium tin oxide, indium zinc oxide, or any combination thereof; an organic film including polyethylene terephthalate, polyethylene naphthalate, polycarbonate, polyimide, polyethylene sulfonate, polyoxymethylene, polyarylate, hexamethyldisiloxane, an acrylic resin (for example, polymethyl methacrylate, polyacrylic acid, and/or the like), an epoxy-based resin (for example, aliphatic glycidyl ether (AGE), and/or the like), or any combination thereof; or a combination of the inorganic film and the organic film.

FIG. 3 is a cross-sectional view of an electronic apparatus according to another embodiment.

The electronic apparatus of FIG. 3 is the same as the light-emitting apparatus of FIG. 2, except that a light-shielding pattern 500 and a functional region 400 are additionally on the encapsulation portion 300. The functional region 400 may be i) a color filter area, ii) a color conversion area, or iii) a combination of the color filter area and the color conversion area. According to an embodiment, the light-emitting device included in the electronic apparatus of FIG. 3 may be a tandem light-emitting device.

### Manufacturing Method

Layers constituting the hole transport region, the emission layer, and the layers constituting the electron transport region may be provided in a certain region by using various suitable methods such as vacuum deposition, spin coating, casting, Langmuir-Blodgett (LB) deposition, ink-jet printing, laser-printing, laser-induced thermal imaging, and/or the like.

When the layers constituting the hole transport region, the emission layer, and the layers constituting the electron transport region are formed by vacuum deposition, the deposition may be performed at a deposition temperature in a range of about 100° C. to about 500° C., at a vacuum degree in a range of about 10⁻⁸ torr to about 10⁻³ torr, and at a deposition speed in a range of about 0.01 Å/sec to about 100 Å/sec, depending on a material to be included in a layer to be formed and the structure of a layer to be formed.

When layers constituting the hole transport region, an emission layer, and layers constituting the electron transport region are formed by spin coating, the spin coating may be performed at a coating speed of about 2,000 rpm to about 5,000 rpm and at a heat treatment temperature of about 80° C. to about 200° C. by taking into account a material to be included in a layer to be formed and the structure of a layer to be formed.

### Definition of Terms

The term “C₃-C₆₀ carbocyclic group” as used herein refers to a cyclic group consisting of carbon atoms as the only ring-forming atoms and having three to sixty carbon atoms, and the term “C₁-C₆₀ heterocyclic group” as used herein refers to a cyclic group that has one to sixty carbon atoms and further includes, in addition to a carbon atom, a heteroatom as a ring-forming atom. The C₃-C₆₀ carbocyclic group and the C₁-C₆₀ heterocyclic group may each be a monocyclic group consisting of one ring or a polycyclic group in which two or more rings are condensed with each other. According to an embodiment, the number of ring-forming atoms of the C₁-C₆₀ heterocyclic group may be 3 to 61.

The “cyclic group” as used herein may include both the C₃-C₆₀ carbocyclic group and the C₁-C₆₀ heterocyclic group.

The term “π electron-rich C₃-C₆₀ cyclic group” as used herein refers to a cyclic group that has three to sixty carbon atoms and does not include *—N═*′ as a ring-forming moiety, and the term “π electron-deficient nitrogen-containing C₁-C₆₀ cyclic group” as used herein refers to a heterocyclic group that has one to sixty carbon atoms and includes *—N═*′ as a ring-forming moiety.

In an embodiment,
the C₃-C₆₀ carbocyclic group may be i) Group T1 or ii) a condensed cyclic group in which two or more of Group T1 are condensed with each other (for example, a cyclopentadiene group, an adamantane group, a norbornane group, a benzene group, a pentalene group, a naphthalene group, an azulene group, an indacene group, an acenaphthylene group, a phenalene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indenophenanthrene group, or an indenoanthracene group),
the C₁-C₆₀ heterocyclic group may be i) Group T2, ii) a condensed cyclic group in which two or more of Group T2 are condensed with each other, or iii) a condensed cyclic group in which at least one Group T2 and at least one Group T1 are condensed with each other (for example, a pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, and/or the like),
the π electron-rich C₃-C₆₀ cyclic group may be i) Group T1, ii) a condensed cyclic group in which two or more of Group T1 are condensed with each other, iii) Group T3, iv) a condensed cyclic group in which two or more of Group T3 are condensed with each other, or v) a condensed cyclic group in which at least one Group T3 and at least one Group T1 are condensed with each other (for example, the C₃-C₆₀ carbocyclic group, a 1H-pyrrole group, a silole group, a borole group, a 2H-pyrrole group, a 3H-pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, or the like),
the π electron-deficient nitrogen-containing C₁-C₆₀ cyclic group may be i) Group T4, ii) a condensed cyclic group in which two or more of Group T4 are condensed with each other, iii) a condensed cyclic group in which at least one Group T4 and at least one Group T1 are condensed with each other, iv) a condensed cyclic group in which at least one Group T4 and at least one Group T3 are condensed with each other, or v) a condensed cyclic group in which at least one Group T4, at least one Group T1, and at least one Group T3 are condensed with one another (for example, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, and the like),
Group T1 may be a cyclopropane group, a cyclobutane group, a cyclopentane group, a cyclohexane group, a cycloheptane group, a cyclooctane group, a cyclobutene group, a cyclopentene group, a cyclopentadiene group, a cyclohexene group, a cyclohexadiene group, a cycloheptene group, an adamantane group, a norbornane (or bicyclo[2.2.1]heptane) group, a norbornene group, a bicyclo[1.1.1]pentane group, a bicyclo[2.1.1]hexane group, a bicyclo[2.2.2]octane group, or a benzene group,
Group T2 may be a furan group, a thiophene group, a 1H-pyrrole group, a silole group, a borole group, a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, a tetrazine group, a pyrrolidine group, an imidazolidine group, a dihydropyrrole group, a piperidine group, a tetrahydropyridine group, a dihydropyridine group, a hexahydropyrimidine group, a tetrahydropyrimidine group, a dihydropyrimidine group, a piperazine group, a tetrahydropyrazine group, a dihydropyrazine group, a tetrahydropyridazine group, or a dihydropyridazine group,
Group T3 may be a furan group, a thiophene group, a 1H-pyrrole group, a silole group, or a borole group, and
Group T4 may be a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, or a tetrazine group.

The terms “cyclic group”, “C₃-C₆₀ carbocyclic group”, “C₁-C₆₀ heterocyclic group”, “Tr electron-rich C₃-C₆₀ cyclic group”, or “π electron-deficient nitrogen-containing C₁-C₆₀ cyclic group” as used herein may refer to a group condensed to any cyclic group, a monovalent group, or a polyvalent group (for example, a divalent group, a trivalent group, a tetravalent group, and/or the like) according to the structure of a formula for which the corresponding term is used. According to an embodiment, “a benzene group” may be a benzo group, a phenyl group, a phenylene group, or the like, which may be easily understand by one of ordinary skill in the art according to the structure of a formula including the “benzene group.”

For example, examples of a monovalent C₃-C₆₀ carbocyclic group and a monovalent C₁-C₆₀ heterocyclic group may include a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group, and examples of a divalent C₃-C₆₀ carbocyclic group and a divalent C₁-C₆₀ heterocyclic group may include a C₃-C₁₀ cycloalkylene group, a C₁-C₁₀ heterocycloalkylene group, a C₃-C₁₀ cycloalkenylene group, a C₁-C₁₀ heterocycloalkenylene group, a C₆-C₆₀ arylene group, a C₁-C₆₀ heteroarylene group, a divalent non-aromatic condensed polycyclic group, and a divalent non-aromatic condensed heteropolycyclic group.

The term “C₁-C₆₀ alkyl group” as used herein refers to a linear or branched aliphatic hydrocarbon monovalent group that has one to sixty carbon atoms, and examples thereof include a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a tert-octyl group, an n-nonyl group, an isononyl group, a sec-nonyl group, a tert-nonyl group, an n-decyl group, an isodecyl group, a sec-decyl group, and a tert-decyl group. The term “C₁-C₆₀ alkylene group” as used herein refers to a divalent group having substantially the same structure as the C₁-C₆₀ alkyl group.

The term “C₂-C₆₀ alkenyl group” as used herein refers to a monovalent hydrocarbon group having at least one carbon-carbon double bond at a main chain (e.g., in the middle) or at a terminal end (e.g., the terminus) of the C₂-C₆₀ alkyl group, and examples thereof include an ethenyl group, a propenyl group, and a butenyl group. The term “C₂-C₆₀ alkenylene group” as used herein refers to a divalent group having substantially the same structure as the C₂-C₆₀ alkenyl group.

The term “C₂-C₆₀ alkynyl group” as used herein refers to a monovalent hydrocarbon group having at least one carbon-carbon triple bond at a main chain (e.g., in the middle) or at a terminal end (e.g., the terminus) of the C₂-C₆₀ alkyl group, and examples thereof include an ethynyl group and a propynyl group. The term “C₂-C₆₀ alkynylene group” as used herein refers to a divalent group having substantially the same structure as the C₂-C₆₀ alkynyl group.

The term “C₁-C₆₀ alkoxy group” as used herein refers to a monovalent group represented by —OA₁₀₁ (wherein A₁₀₁ is the C₁-C₆₀ alkyl group), and examples thereof include a methoxy group, an ethoxy group, and an isopropyloxy group.

The term “C₃-C₁₀ cycloalkyl group” as used herein refers to a monovalent saturated hydrocarbon monocyclic group including 3 to 10 carbon atoms. Examples of the C₃-C₁₀ cycloalkyl group as used herein include a cyclopropyl group, a cyclobutyl group, a cyclopentyl group, a cyclohexyl group, a cycloheptyl group, a cyclooctyl group, an adamantyl group, a norbornyl (bicyclo[2.2.1]heptyl) group, a bicyclo[1.1.1]pentyl group, a bicyclo[2.1.1]hexyl group, or a bicyclo[2.2.2]octyl group. The term “C₃-C₁₀ cycloalkylene group” as used herein refers to a divalent group having the same structure as the C₃-C₁₀ cycloalkyl group.

The term “C₁-C₁₀ heterocycloalkyl group” as used herein refers to a monovalent cyclic group that has one to ten carbon atoms and further includes, in addition to the carbon atoms, at least one heteroatom as a ring-forming atom, and examples thereof include a 1,2,3,4-oxatriazolidinyl group, a tetrahydrofuranyl group, and a tetrahydrothiophenyl group. The term “C₁-C₁₀ heterocycloalkylene group” as used herein refers to a divalent group having substantially the same structure as the C₁-C₁₀ heterocycloalkyl group.

The term “C₃-C₁₀ cycloalkenyl group” as used herein refers to a monovalent cyclic group that has three to ten carbon atoms and at least one carbon-carbon double bond in the ring thereof and no aromaticity (e.g., is not aromatic), and examples thereof include a cyclopentenyl group, a cyclohexenyl group, and a cycloheptenyl group. The term “C₃-C₁₀ cycloalkenylene group” as used herein refers to a divalent group having substantially the same structure as the C₃-C₁₀ cycloalkenyl group.

The term “C₁-C₁₀ heterocycloalkenyl group” as used herein refers to a monovalent cyclic group that has one to ten carbon atoms, further includes, in addition to the carbon atoms, at least one heteroatom as a ring-forming atom, and has at least one double bond in the ring thereof. Examples of the C₁-C₁₀ heterocycloalkenyl group are a 4,5-dihydro-1,2,3,4-oxatriazolyl group, a 2,3-dihydrofuranyl group, and a 2,3-dihydrothiophenyl group. The term “C₁-C₁₀ heterocycloalkenylene group” as used herein refers to a divalent group having the same structure as the C₁-C₁₀ heterocycloalkenyl group.

The term “C₆-C₆₀ aryl group” as used herein refers to a monovalent group having a carbocyclic aromatic system of six to sixty carbon atoms, and the term “C₆-C₆₀ arylene group” as used herein refers to a divalent group having a carbocyclic aromatic system of six to sixty carbon atoms. Examples of the C₆-C₆₀ aryl group are a phenyl group, a pentalenyl group, a naphthyl group, an azulenyl group, an indacenyl group, an acenaphthyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a heptalenyl group, a naphthacenyl group, a picenyl group, a hexacenyl group, a pentacenyl group, a rubicenyl group, a coronenyl group, and an ovalenyl group. When the C₆-C₆₀ aryl group and the C₆-C₆₀ arylene group each include two or more rings, the two or more rings may be condensed with each other.

The term “C₁-C₆₀ heteroaryl group” as used herein refers to a monovalent group having a heterocyclic aromatic system that has one to sixty carbon atoms and further includes, in addition to the carbon atoms, at least one heteroatom as a ring-forming atom. The term “C₁-C₆₀ heteroarylene group” as used herein refers to a divalent group having a heterocyclic aromatic system that has one to sixty carbon atoms and further includes, in addition to the carbon atoms, at least one heteroatom as a ring-forming atom. Examples of the C₁-C₆₀ heteroaryl group are a pyridinyl group, a pyrimidinyl group, a pyrazinyl group, a pyridazinyl group, a triazinyl group, a quinolinyl group, a benzoquinolinyl group, an isoquinolinyl group, a benzoisoquinolinyl group, a quinoxalinyl group, a benzoquinoxalinyl group, a quinazolinyl group, a benzoquinazolinyl group, a cinnolinyl group, a phenanthrolinyl group, a phthalazinyl group, and a naphthyridinyl group. When the C₁-C₆₀ heteroaryl group and the C₁-C₆₀ heteroarylene group each include two or more rings, the two or more rings may be condensed with each other.

The term “monovalent non-aromatic condensed polycyclic group” as used herein refers to a monovalent group having two or more rings condensed with each other, only carbon atoms (for example, eight to sixty carbon atoms) as ring-forming atoms, and no aromaticity in its molecular structure when considered as a whole (e.g., is not aromatic when considered as a whole). Examples of the monovalent non-aromatic condensed polycyclic group include an indenyl group, a fluorenyl group, a spiro-bifluorenyl group, a benzofluorenyl group, an indenophenanthrenyl group, and an indeno anthracenyl group. The term “divalent non-aromatic condensed polycyclic group” as used herein refers to a divalent group having substantially the same structure as the monovalent non-aromatic condensed polycyclic group.

The term “monovalent non-aromatic condensed heteropolycyclic group” as used herein refers to a monovalent group that has two or more rings condensed with each other, further includes, in addition to carbon atoms (for example, one to sixty carbon atoms), at least one heteroatom as a ring-forming atom, and has no aromaticity in its molecular structure when considered as a whole (e.g., is not aromatic when considered as a whole). Examples of the monovalent non-aromatic condensed heteropolycyclic group are a pyrrolyl group, a thiophenyl group, a furanyl group, an indolyl group, a benzoindolyl group, a naphtho indolyl group, an isoindolyl group, a benzoisoindolyl group, a naphthoisoindolyl group, a benzosilolyl group, a benzothiophenyl group, a benzofuranyl group, a carbazolyl group, a dibenzosilolyl group, a dibenzothiophenyl group, a dibenzofuranyl group, an azacarbazolyl group, an azafluorenyl group, an azadibenzosilolyl group, an azadibenzothiophenyl group, an azadibenzofuranyl group, a pyrazolyl group, an imidazolyl group, a triazolyl group, a tetrazolyl group, an oxazolyl group, an isoxazolyl group, a thiazolyl group, an isothiazolyl group, an oxadiazolyl group, a thiadiazolyl group, a benzopyrazolyl group, a benzimidazolyl group, a benzoxazolyl group, a benzothiazolyl group, a benzoxadiazolyl group, a benzothiadiazolyl group, an imidazopyridinyl group, an imidazopyrimidinyl group, an imidazotriazinyl group, an imidazopyrazinyl group, an imidazopyridazinyl group, an indenocarbazolyl group, an indolocarbazolyl group, a benzofurocarbazolyl group, a benzothienocarbazolyl group, a benzosilolocarbazolyl group, a benzoindolocarbazolyl group, a benzocarbazolyl group, a benzonaphthofuranyl group, a benzonaphthothiophenyl group, a benzonaphthosilolyl group, a benzofurodibenzofuranyl group, a benzofurodibenzothiophenyl group, a benzothienodibenzothiophenyl group, and the like. The term “divalent non-aromatic condensed heteropolycyclic group” as used herein refers to a divalent group having substantially the same structure as the monovalent non-aromatic condensed heteropolycyclic group.

The term “C₆-C₆₀ aryloxy group” as used herein refers to —OA₁₀₂ (wherein A₁₀₂ is the C₆-C₆₀ aryl group), and the term “C₆-C₆₀ arylthio group” as used herein refers to —SA₁₀₃ (wherein A₁₀₃ is the C₆-C₆₀ aryl group).

The term “C₇-C₆₀ arylalkyl group” as used herein refers to -A₁₀₄A₁₀₅ (wherein A₁₀₄ is a C₁-C₅₄ alkylene group, and A₁₀₅ is a C₆-C₅₉ aryl group), and the term “C₂-C₆₀ heteroarylalkyl group” as used herein refers to -A₁₀₆A₁₀₇ (wherein A₁₀₆ is a C₁-C₅₉ alkylene group, and A₁₀₇ is a C₁-C₅₉ heteroaryl group).

The term “R₁₀ₐ” as used herein may be:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂).
Q₁ to Q₃, Q₁₁ to Q₁₃, Q₂₁ to Q₂₃, and Q₃₁ to Q₃₃ may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or
a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, cyano group, a C₁-C₆₀ alkyl group, a C₁-C₆₀ alkoxy group, a phenyl group, a biphenyl group, or any combination thereof.

The term “heteroatom” as used herein refers to any atom other than a carbon atom. Examples of the heteroatom include O, S, N, P, Si, B, Ge, Se, or any combination thereof.

The term “transition metal” as used herein may include Hf, Ta, W, Re, Os, Ir, Pt, Au, and/or the like.

The term “Ph” as used herein refers to a phenyl group, the term “Me” as used herein refers to a methyl group, the term “Et” as used herein refers to an ethyl group, the term “ter-Bu” or “Bu^{t}” as used herein refers to a tert-butyl group, and the term “OMe” as used herein refers to a methoxy group.

The term “biphenyl group” as used herein refers to “a phenyl group substituted with a phenyl group.” In embodiments, the “biphenyl group” is a substituted phenyl group having a C₆-C₆₀ aryl group as a substituent.

The term “terphenyl group” as used herein refers to “a phenyl group substituted with a biphenyl group”. In embodiments, the “terphenyl group” is a substituted phenyl group having, as a substituent, a C₆-C₆₀ aryl group substituted with a C₆-C₆₀ aryl group.

The number of carbon atoms in the substituent definition is merely an example. For example, in the C₁-C₆₀ alkyl group, the number of carbon atoms, 60, is an example, and the definition for the alkyl group is equally applied to the C₁-C₂₀ alkyl group. The same applies to other embodiments.

Any hydrogen in the compound structures described herein may optionally be substituted with deuterium.

* and *′ as used herein, unless defined otherwise, each refer to a binding site to a neighboring atom in a corresponding formula.

Hereinafter, a light-emitting device according to embodiments will be described in more detail with reference to Examples.

### EXAMPLES

### Synthesis Example 1 (Compound 1)

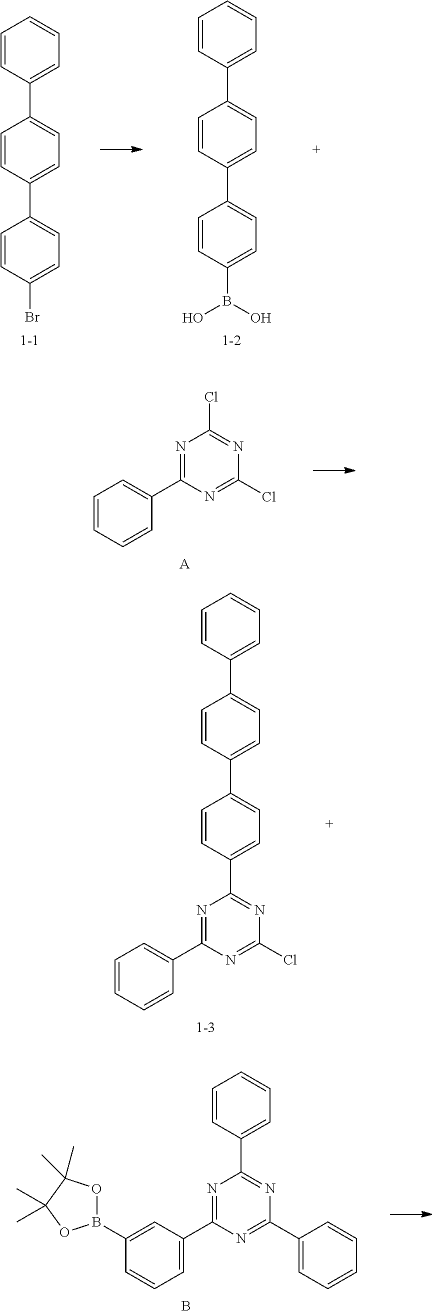

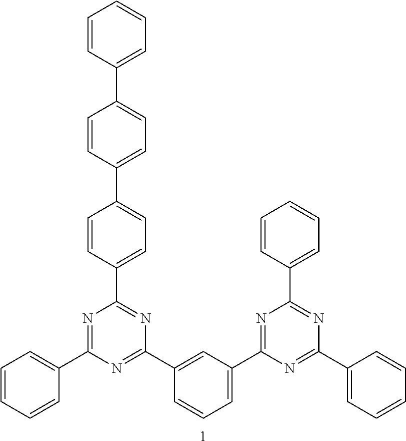

### Synthesis of Intermediate Compound 1-2

Compound 1-1 (1.0 eq.) was added to THE and cooled to −78° C. Under a nitrogen atmosphere, n-butyllithium (1.05 eq.) was slowly added dropwise, and the mixture was stirred at −78° C. for 1 hour. Trimethyl borate (1.2 eq.) was slowly added dropwise to the resultant solution at −78° C., and after stirring at room temperature for 4 hours, HCl was added dropwise and stirred at room temperature for 2 hours. After washing with ethyl acetate and water three times, the obtained organic layer was dried over MgSO₄ and dried under reduced pressure. The obtained solid was washed with water and methanol, filtered to obtain Intermediate Compound 1-2. (Yield: 68%)

### Synthesis of Intermediate Compound 1-3

Intermediate Compound 1-2 (1.0 eq.), 2,4-dichloro-6-phenyl-1,3,5-triazine (Compound A, 1.1 eq.), bis(triphenylphosphine)palladium(II)dichloride (0.02 eq.), and potassium carbonate (2.0 eq.) were dissolved in toluene:EtOH:H₂O (4:1:1 volume ratio) and stirred at 100° C. under a nitrogen atmosphere for 5 hours. After cooling, the resultant mixture was washed with ethyl acetate and water three times, and the obtained organic layer was dried over MgSO₄ and dried under reduced pressure. Intermediate Compound 1-3 was obtained by column chromatography. (Yield: 73%)

### Synthesis of Compound 1

Intermediate Compound 1-3 (1.0 eq.), 2,4-diphenyl-6-(3-(4,4,5,5-tetramethyl-1,3,2-dioxaborolan-2-yl)phenyl)-1,3,5-triazine (Compound B, 1.2 eq.), tetrakis(triphenylphosphine)palladium (0.05 eq.), and potassium carbonate (2.0 eq.) were dissolved in toluene:EtOH:H₂O (4:1:1 volume ratio) and stirred at 100° C. under a nitrogen atmosphere for 5 hours. After cooling, the resultant mixture was washed with ethyl acetate and water three times, and the obtained organic layer was dried over MgSO₄ and dried under reduced pressure. Compound 1 was obtained by column chromatography. (Yield: 73%)

### Synthesis Example 2 (Compound 6)

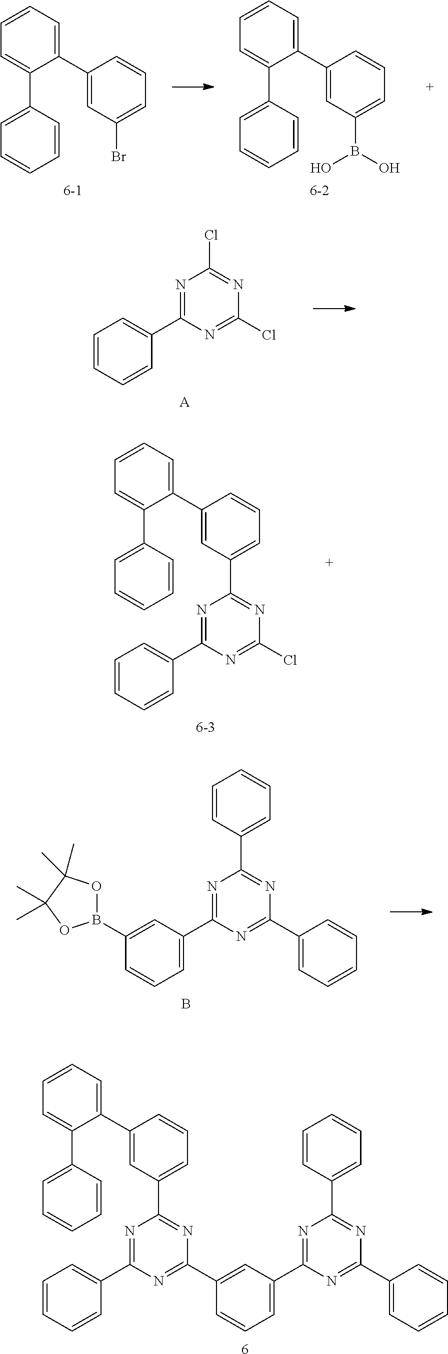

### Synthesis of Intermediate Compound 6-2

Compound 6-1 (1.0 eq.) was added to THE and cooled to −78° C. Under a nitrogen atmosphere, n-butyllithium (1.05 eq.) was slowly added dropwise, and the mixture was stirred at −78° C. for 1 hour. Trimethyl borate (1.2 eq.) was slowly added dropwise to the resultant solution at −78° C., and after stirring at room temperature for 4 hours, HCl was added dropwise and stirred at room temperature for 2 hours. After washing with ethyl acetate and water three times, the obtained organic layer was dried over MgSO₄ and dried under reduced pressure. The obtained solid was washed with water and methanol, filtered to obtain Intermediate Compound 6-2. (Yield: 69%)

### Synthesis of Intermediate Compound 6-3

Intermediate Compound 6-2 (1.0 eq.), 2,4-dichloro-6-phenyl-1,3,5-triazine (Compound A, 1.1 eq.), bis(triphenylphosphine)palladium(II)dichloride (0.02 eq.), and potassium carbonate (2.0 eq.) were dissolved in toluene:EtOH:H₂O (4:1:1 volume ratio) and stirred at 100° C. under a nitrogen atmosphere for 5 hours. After cooling, the resultant mixture was washed with ethyl acetate and water three times, and the obtained organic layer was dried over MgSO₄ and dried under reduced pressure. Intermediate Compound 6-3 was obtained by column chromatography. (Yield: 74%)

### Synthesis of Compound 6

Intermediate Compound 6-3 (1.0 eq.), 2,4-diphenyl-6-(3-(4,4,5,5-tetramethyl-1,3,2-dioxaborolan-2-yl)phenyl)-1,3,5-triazine (Compound B, 1.2 eq.), tetrakis(triphenylphosphine)palladium (0.05 eq.), and potassium carbonate (2.0 eq.) were dissolved in toluene:EtOH:H₂O (4:1:1 volume ratio) and stirred at 100° C. under a nitrogen atmosphere for 5 hours. After cooling, the resultant mixture was washed with ethyl acetate and water three times, and the obtained organic layer was dried over MgSO₄ and dried under reduced pressure. Compound 6 was obtained by column chromatography. (Yield: 74%)

### Synthesis Example 3 (Compound 7)

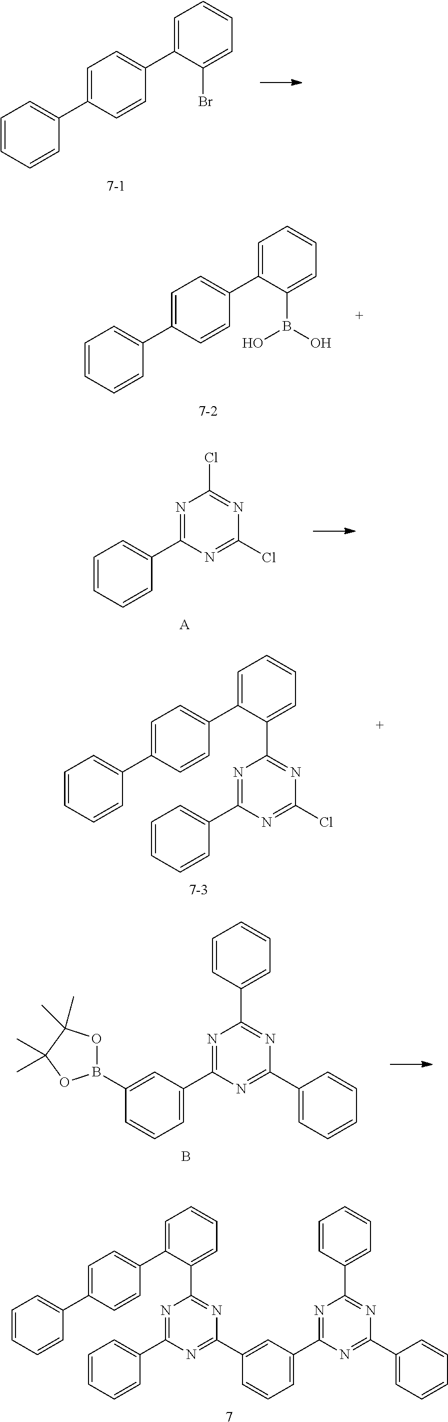

### Synthesis of Intermediate Compound 7-2

Compound 7-1 (1.0 eq.) was added to THE and cooled to −78° C. Under a nitrogen atmosphere, n-butyllithium (1.05 eq.) was slowly added dropwise, and the mixture was stirred at −78° C. for 1 hour. Trimethyl borate (1.2 eq.) was slowly added dropwise to the resultant solution at −78° C., and after stirring at room temperature for 4 hours, HCl was added dropwise and stirred at room temperature for 2 hours. After washing with ethyl acetate and water three times, the obtained organic layer was dried over MgSO₄ and dried under reduced pressure. The obtained solid was washed with water and methanol, filtered to obtain Intermediate Compound 7-2. (Yield: 63%)

### Synthesis of Intermediate Compound 7-3

Intermediate Compound 7-2 (1.0 eq.), 2,4-dichloro-6-phenyl-1,3,5-triazine (Compound A, 1.1 eq.), bis(triphenylphosphine)palladium(II)dichloride (0.02 eq.), and potassium carbonate (2.0 eq.) were dissolved in toluene:EtOH:H₂O (4:1:1 volume ratio) and stirred at 100° C. under a nitrogen atmosphere for 5 hours. After cooling, the resultant mixture was washed with ethyl acetate and water three times, and the obtained organic layer was dried over MgSO₄ and dried under reduced pressure. Intermediate Compound 7-3 was obtained by column chromatography. (Yield: 73%)

### Synthesis of Intermediate Compound 7

Intermediate Compound 7-3 (1.0 eq.), 2,4-diphenyl-6-(3-(4,4,5,5-tetramethyl-1,3,2-dioxaborolan-2-yl)phenyl)-1,3,5-triazine (Compound B, 1.2 eq.), tetrakis(triphenylphosphine)palladium (0.05 eq.), and potassium carbonate (2.0 eq.) were dissolved in toluene:EtOH:H₂O (4:1:1 volume ratio) and stirred at 100° C. under a nitrogen atmosphere for 5 hours. After cooling, the resultant mixture was washed with ethyl acetate and water three times, and the obtained organic layer was dried over MgSO₄ and dried under reduced pressure. Compound 7 was obtained by column chromatography. (Yield: 73%)

### Synthesis Example 4 (Compound 9)

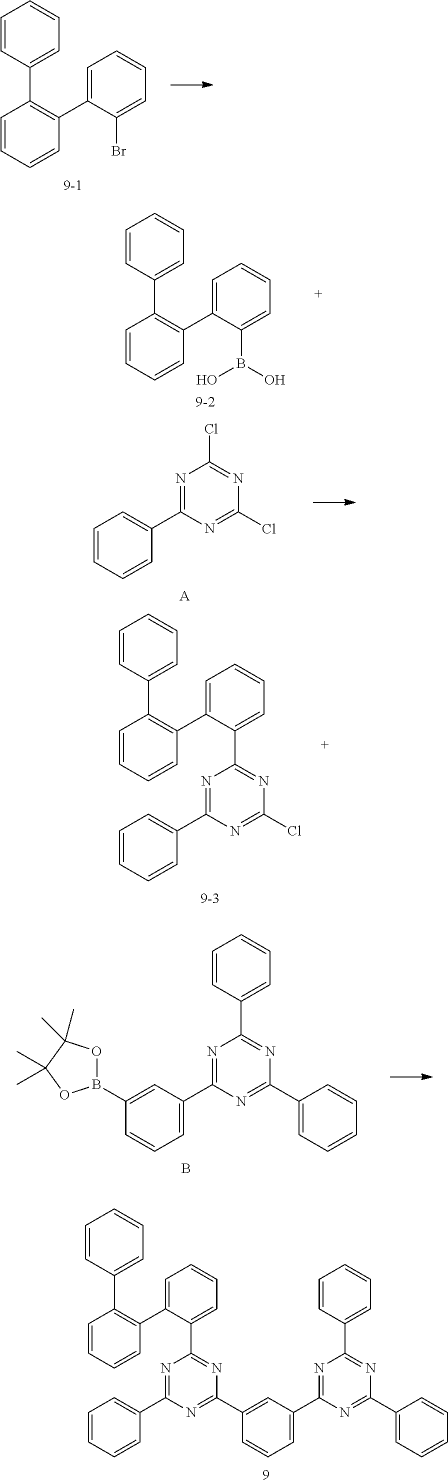

### Synthesis of Intermediate Compound 9-2

Compound 9-1 (1.0 eq.) was added to THE and cooled to −78° C. Under a nitrogen atmosphere, n-butyllithium (1.05 eq.) was slowly added dropwise, and the resultant mixture was stirred at −78° C. for 1 hour. Trimethyl borate (1.2 eq.) was slowly added dropwise to the solution at −78° C., and after stirring at room temperature for 4 hours, HCl was added dropwise and stirred at room temperature for 2 hours. After washing with ethyl acetate and water three times, the obtained organic layer was dried over MgSO₄ and dried under reduced pressure. The obtained solid was washed with water and methanol, filtered to obtain Intermediate Compound 9-2. (Yield: 68%)

### Synthesis of Intermediate Compound 9-3

Intermediate Compound 9-2 (1.0 eq.), 2,4-dichloro-6-phenyl-1,3,5-triazine (Compound A, 1.1 eq.), bis(triphenylphosphine)palladium(II)dichloride (0.02 eq.), and potassium carbonate (2.0 eq.) were dissolved in toluene:EtOH:H₂O (4:1:1 volume ratio) and stirred at 100° C. under a nitrogen atmosphere for 5 hours. After cooling, the resultant mixture was washed with ethyl acetate and water three times, and the obtained organic layer was dried over MgSO₄ and dried under reduced pressure. Intermediate Compound 9-3 was obtained by column chromatography. (Yield: 75%)

### Synthesis of Compound 9

Intermediate Compound 9-3 (1.0 eq.), 2,4-diphenyl-6-(3-(4,4,5,5-tetramethyl-1,3,2-dioxaborolan-2-yl)phenyl)-1,3,5-triazine (Compound B, 1.2 eq.), tetrakis(triphenylphosphine)palladium (0.05 eq.), and potassium carbonate (2.0 eq.) were dissolved in toluene:EtOH:H₂O (4:1:1 volume ratio) and stirred at 100° C. under a nitrogen atmosphere for 5 hours. After cooling, the resultant reaction solution was washed three times with ethyl acetate and water, and the resulting organic layer was dried with MgSO₄ first and then dried again under reduced pressure. Compound 9 was obtained by column chromatography. (Yield: 73%)

### Synthesis Example 5 (Compound 12)

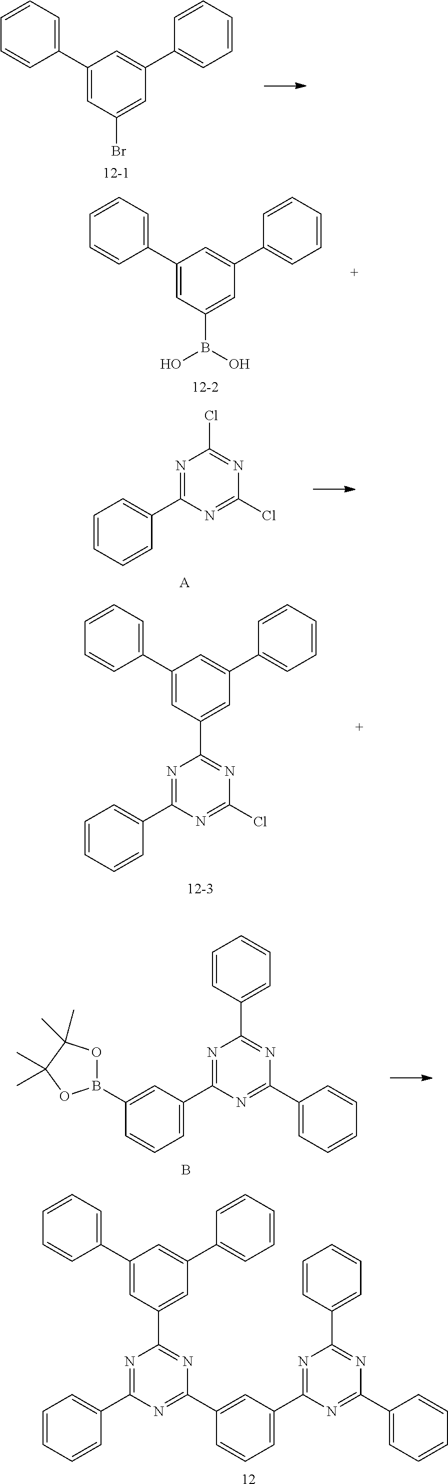

### Synthesis of Intermediate Compound 12-2

Compound 12-1 (1.0 eq.) was added to THE and cooled to −78° C. Under a nitrogen atmosphere, n-butyllithium (1.05 eq.) was slowly added dropwise, and the resultant mixture was stirred at −78° C. for 1 hour. Trimethyl borate (1.2 eq.) was slowly added dropwise to the solution at −78° C., and after stirring at room temperature for 4 hours, HCl was added dropwise and stirred at room temperature for 2 hours. The resultant reaction solution was washed three times with ethyl acetate and water, and the resulting organic layer was dried with MgSO₄ first and then dried again under reduced pressure. The obtained solid was washed with water and methanol, filtered to obtain Intermediate Compound 12-2. (Yield: 68%)

### Synthesis of Intermediate Compound 12-3

Intermediate Compound 12-2 (1.0 eq.), 2,4-dichloro-6-phenyl-1,3,5-triazine (Compound A, 1.1 eq.), bis(triphenylphosphine)palladium(II)dichloride (0.02 eq.), and potassium carbonate (2.0 eq.) were dissolved in toluene:EtOH:H₂O (4:1:1 volume ratio) and stirred at 100° C. under a nitrogen atmosphere for 5 hours. After cooling, the resultant reaction solution was washed three times with ethyl acetate and water, and the resulting organic layer was dried with MgSO₄ first and then dried again under reduced pressure. Intermediate Compound 12-3 was obtained by column chromatography. (Yield: 83%)

### Synthesis of Compound 12

Intermediate Compound 12-3 (1.0 eq.), 2,4-diphenyl-6-(3-(4,4,5,5-tetramethyl-1,3,2-dioxaborolan-2-yl)phenyl)-1,3,5-triazine (Compound B, 1.2 eq.), tetrakis(triphenylphosphine)palladium (0.05 eq.), and potassium carbonate (2.0 eq.) were dissolved in toluene:EtOH:H₂O (4:1:1 volume ratio) and stirred at 100° C. under a nitrogen atmosphere for 5 hours. After cooling, the resultant reaction solution was washed three times with ethyl acetate and water, and the resulting organic layer was dried with MgSO₄ first and then dried again under reduced pressure. Compound 12 was obtained by column chromatography. (Yield: 71%)

### Synthesis Example 6 (Compound 14)

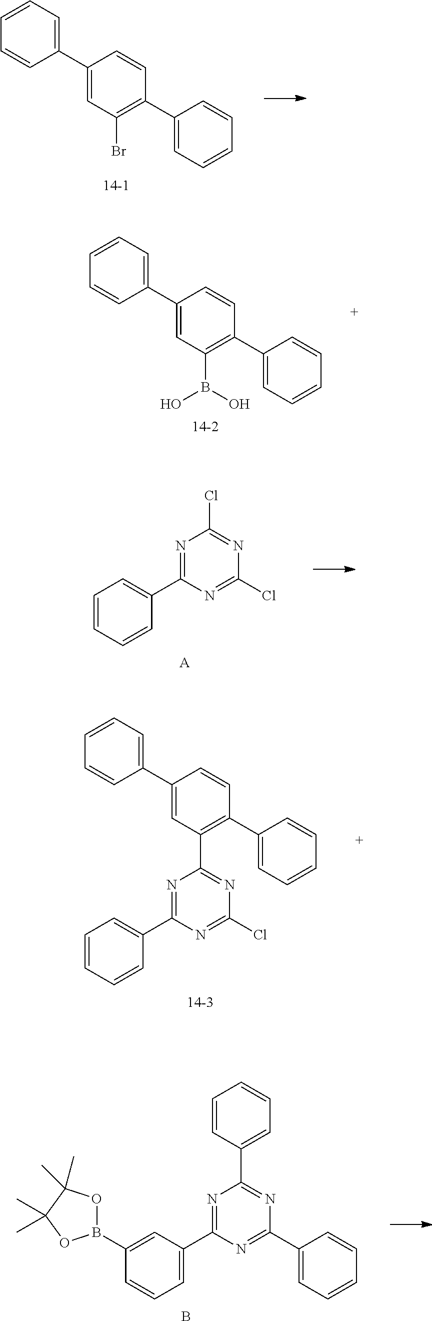

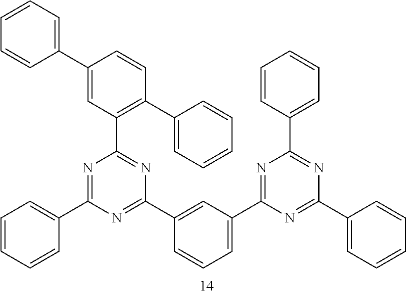

### Synthesis of Intermediate Compound 14-2

Compound 14-1 (1.0 eq.) was added to THE and cooled to −78° C. Under a nitrogen atmosphere, n-butyllithium (1.05 eq.) was slowly added dropwise, and the resultant mixture was stirred at −78° C. for 1 hour. Trimethyl borate (1.2 eq.) was slowly added dropwise to the solution at −78° C., and after stirring at room temperature for 4 hours, HCl was added dropwise and stirred at room temperature for 2 hours. The resultant reaction solution was washed three times with ethyl acetate and water, and the resulting organic layer was dried with MgSO₄ first and then dried again under reduced pressure. The obtained solid was washed with water and methanol, filtered to obtain Intermediate Compound 14-2. (Yield: 68%)

### Synthesis of Intermediate Compound 14-3

Intermediate Compound 14-2 (1.0 eq.), 2,4-dichloro-6-phenyl-1,3,5-triazine (Compound A, 1.1 eq.), bis(triphenylphosphine)palladium(II)dichloride (0.02 eq.), and potassium carbonate (2.0 eq.) were dissolved in toluene:EtOH:H₂O (4:1:1 volume ratio) and stirred at 100° C. under a nitrogen atmosphere for 5 hours. After cooling, the resultant reaction solution was washed three times with ethyl acetate and water, and the resulting organic layer was dried with MgSO₄ first and then dried again under reduced pressure. Intermediate Compound 14-3 was obtained by column chromatography. (Yield: 82%)

### Synthesis of Compound 14

Intermediate Compound 14-3 (1.0 eq.), 2,4-diphenyl-6-(3-(4,4,5,5-tetramethyl-1,3,2-dioxaborolan-2-yl)phenyl)-1,3,5-triazine (Compound B, 1.2 eq.), tetrakis(triphenylphosphine)palladium (0.05 eq.), and potassium carbonate (2.0 eq.) were dissolved in toluene:EtOH:H₂O (4:1:1 volume ratio) and stirred at 100° C. under a nitrogen atmosphere for 5 hours. After cooling, the resultant reaction solution was washed three times with ethyl acetate and water, and the resulting organic layer was dried with MgSO₄ first and then dried again under reduced pressure. Compound 14 was obtained by column chromatography. (Yield: 71%)

### Synthesis Example 7 (Compound 15)

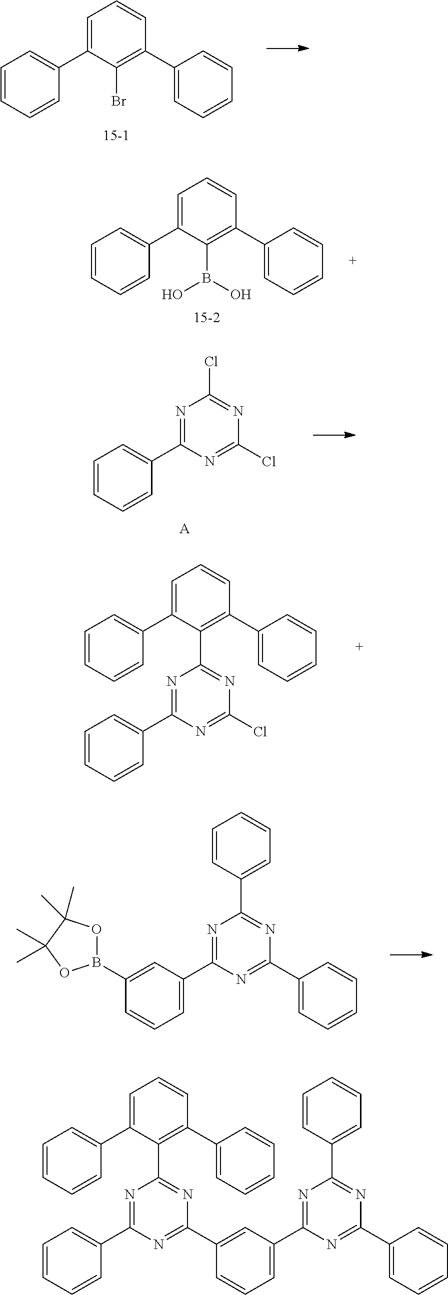

### Synthesis of Intermediate Compound 15-2

Compound 15-1 (1.0 eq.) was added to THE and cooled to −78° C. Under a nitrogen atmosphere, n-butyllithium (1.05 eq.) was slowly added dropwise, and the resultant mixture was stirred at −78° C. for 1 hour. Trimethyl borate (1.2 eq.) was slowly added dropwise to the solution at −78° C., and after stirring at room temperature for 4 hours, HCl was added dropwise and stirred at room temperature for 2 hours. The resultant reaction solution was washed three times with ethyl acetate and water, and the resulting organic layer was dried with MgSO₄ first and then dried again under reduced pressure. The obtained solid was washed with water and methanol, filtered to obtain Intermediate Compound 15-2. (Yield: 68%)

### Synthesis of Intermediate Compound 15-3

Intermediate Compound 15-2 (1.0 eq.), 2,4-dichloro-6-phenyl-1,3,5-triazine (Compound A, 1.1 eq.), bis(triphenylphosphine)palladium(II)dichloride (0.02 eq.), and potassium carbonate (2.0 eq.) were dissolved in toluene:EtOH:H₂O (4:1:1 volume ratio) and stirred at 100° C. under a nitrogen atmosphere for 5 hours. After cooling, the resultant reaction solution was washed three times with ethyl acetate and water, and the resulting organic layer was dried with MgSO₄ first and then dried again under reduced pressure. Intermediate Compound 15-3 was obtained by column chromatography. (Yield: 82%)

### Synthesis of Compound 15

Intermediate Compound 15-3 (1.0 eq.), 2,4-diphenyl-6-(3-(4,4,5,5-tetramethyl-1,3,2-dioxaborolan-2-yl)phenyl)-1,3,5-triazine (Compound B, 1.2 eq.), tetrakis(triphenylphosphine)palladium (0.05 eq.), and potassium carbonate (2.0 eq.) were dissolved in toluene:EtOH:H₂O (4:1:1 volume ratio) and stirred at 100° C. under a nitrogen atmosphere for 5 hours. After cooling, the resultant reaction solution was washed three times with ethyl acetate and water, and the resulting organic layer was dried with MgSO₄ first and then dried again under reduced pressure. Compound 15 was obtained by column chromatography. (Yield: 71%)

### Synthesis Example 8 (Compound 181)

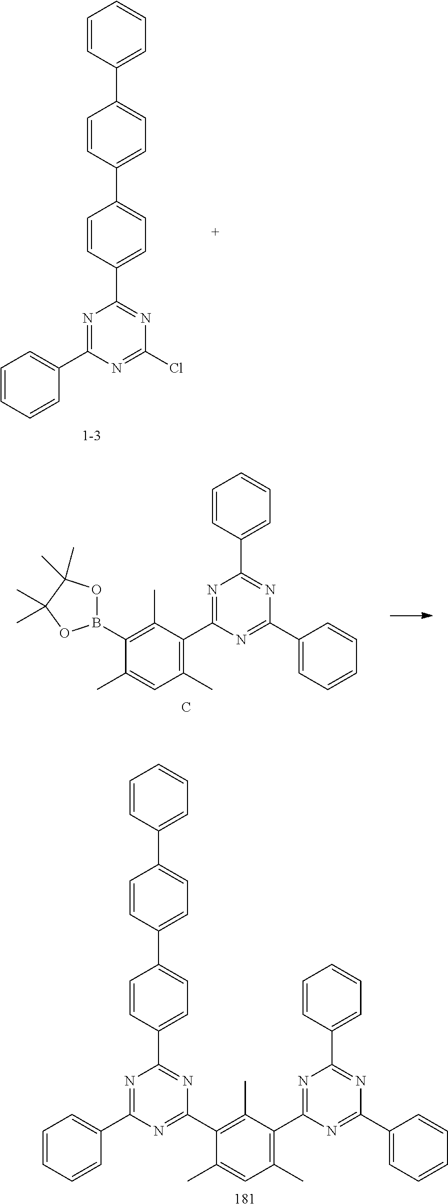

### Synthesis of Compound 181

Intermediate Compound 1-3 (1.0 eq.), 2,4-diphenyl-6-(2,4,6-trimethyl-3-(4,4,5,5-tetramethyl-1,3,2-dioxaborolan-2-yl)phenyl)-1,3,5-triazine (Compound C, 1.2 eq.), tetrakis(triphenylphosphine)palladium (0.05 eq.), and potassium carbonate (2.0 eq.) were dissolved in toluene:EtOH:H₂O (4:1:1 volume ratio) and stirred at 100° C. under a nitrogen atmosphere for 5 hours. After cooling, the resultant reaction solution was washed three times with ethyl acetate and water, and the resulting organic layer was dried with MgSO₄ first and then dried again under reduced pressure. Compound 181 was obtained by column chromatography. (Yield: 72%)

### Synthesis Example 9 (Compound 186)

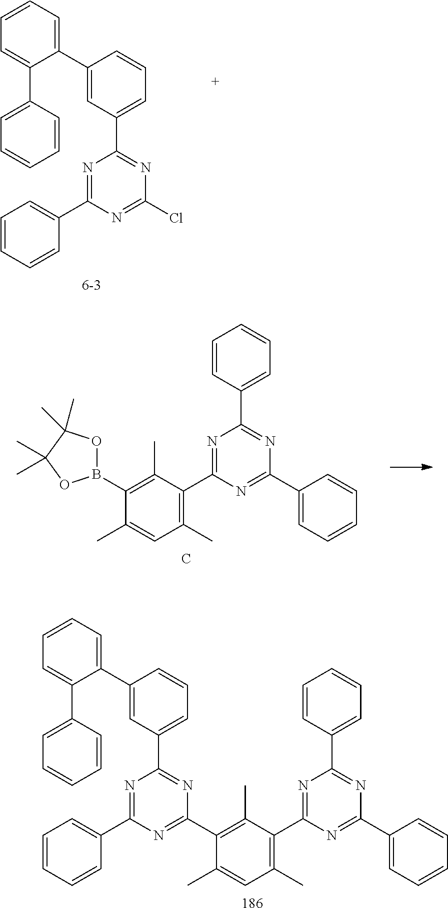

### Synthesis of Compound 186

Intermediate Compound 6-3 (1.0 eq.), 2,4-diphenyl-6-(2,4,6-trimethyl-3-(4,4,5,5-tetramethyl-1,3,2-dioxaborolan-2-yl)phenyl)-1,3,5-triazine (Compound C, 1.2 eq.), tetrakis(triphenylphosphine)palladium (0.05 eq.), and potassium carbonate (2.0 eq.) were dissolved in toluene:EtOH:H₂O (4:1:1 volume ratio) and stirred at 100° C. under a nitrogen atmosphere for 5 hours. After cooling, the resultant reaction solution was washed three times with ethyl acetate and water, and the resulting organic layer was dried with MgSO₄ first and then dried again under reduced pressure. Compound 186 was obtained by column chromatography. (Yield: 73%)

### Synthesis Example 10 (Compound 192)

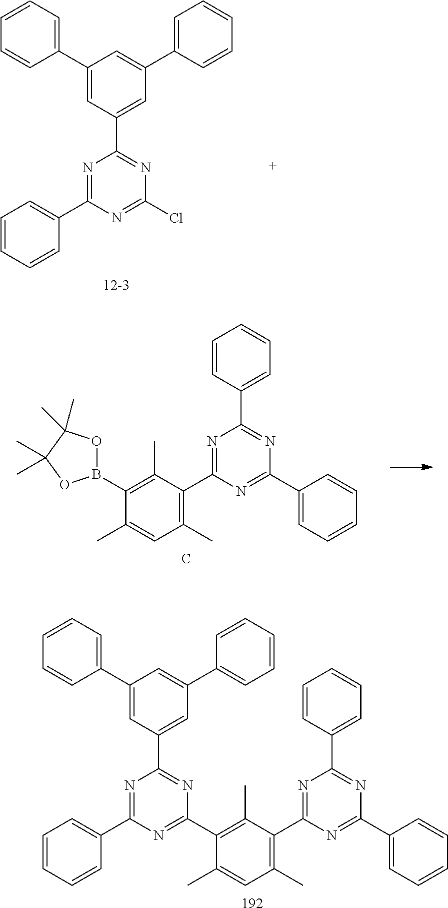

### Synthesis of Compound 192

Intermediate Compound 12-3 (1.0 eq.), 2,4-diphenyl-6-(2,4,6-trimethyl-3-(4,4,5,5-tetramethyl-1,3,2-dioxaborolan-2-yl)phenyl)-1,3,5-triazine (Compound C, 1.2 eq.), tetrakis(triphenylphosphine)palladium (0.05 eq.), and potassium carbonate (2.0 eq.) were dissolved in toluene:EtOH:H₂O (4:1:1 volume ratio) and stirred at 100° C. under a nitrogen atmosphere for 5 hours. After cooling, the resultant reaction solution was washed three times with ethyl acetate and water, and the resulting organic layer was dried with MgSO₄ first and then dried again under reduced pressure. Compound 192 was obtained by column chromatography. (Yield: 74%)

For the compounds synthesized in Synthesis Examples 1 to 10, ¹H NMR and high-resolution mass (HR-MS) were measured. Results are shown in Table 1. Synthesis methods of compounds other than the compounds of Synthesis Examples 1 to 10 may be easily recognized by those skilled in the art by referring to the synthesis paths and source materials.

TABLE 1

| | | HR-MS (m/z)[M⁺] | |
|---|---|---|---|
| Compound | ¹H NMR (CDCl₃, 500 MHz) | found | calc. |
| Compound | 8.38-8.36(m, 8H), 7.96-7.94(m, 3H), 7.75- | 692.27 | 692.83 |
| 1 | 7.73(m, 3H), 7.50-7.41(m, 12H), 7.25(m, 6H) | | |
| Compound | 8.38-8.36(m, 9H), 7.96-7.94(m, 4H), 7.79- | 692.27 | 692.83 |
| 6 | 7.73(m, 4H), 7.61-7.60(m, 3H), 7.50-7.46(m, | | |
| | 12H) | | |
| Compound | 8.38-8.36(m, 8H), 7.96-7.94(m, 3H), 7.75- | 692.27 | 692.83 |
| 7 | 7.73(m, 3H), 7.60(d. 2H), 7.50-7.41(m, 12H), | | |
| | 7.25(d, 4H) | | |
| Compound | 8.38-8.36(m, 8H), 7.96-7.94(m, 5H), 7.79- | 692.27 | 692.83 |
| 9 | 7.73(m, 3H), 7.60(m, 4H), 7.50-7.41(m, 12H) | | |
| Compound | 8.38-8.36(m, 8H), 8.04(s, 3H), 7.94(s, 1H), | 692.27 | 692.83 |
| 12 | 7.75-7.73(m, 5H), 7.50-7.41(m, 15H) | | |
| Compound | 8.38-8.36(m, 8H), 8.15-8.13(m, 2H), 7.94(s, | 692.27 | 692.83 |
| 14 | 1H), 7.79-7.75(m, 5H), 7.50-7.35(m, 16H) | | |
| Compound | 8.38-8.36(m, 8H), 8.06(d, 2H), 7.94(s, 1H), | 692.27 | 692.83 |
| 15 | 7.83-7.73(m, 6H), 7.50-7.46(m, 15H) | | |
| Compound | 8.36(m, 6H), 7.96(d, 2H), 7.75(m, 2H), 7.50- | 734.32 | 734.91 |
| 181 | 7.41(m, 12H), 7.35(s, 1H), 7.25(m, 6H), | | |
| | 2.57(s, 6H), 2.33(s, 3H) | | |
| Compound | 8.38-8.36(m, 7H), 7.96-7.94(d, 3H), 7.79- | 734.32 | 734.91 |
| 186 | 7.73(m, 3H), 7.61-7.60(m, 3H), 7.50-7.46(m, | | |
| | 12H), 7.35(s, 1H), 2.57(s, 6H), 2.33(s, 3H) | | |
| Compound | 8.36(m, 6H), 8.04(s, 3H), 7.75(m, 4H), 7.50- | 734.32 | 734.91 |
| 192 | 7.49(m, 15H), 7.35(s, 1H), 2.57(s, 6H), 2.33(s, | | |
| | 3H) | | |

### Manufacture of Light-Emitting Devices

### Comparative Example 1

A glass substrate (Corning) with an ITO electrode having 15 Ω/cm² (1200 Å) formed as an anode was cut to a size of 50 mm×50 mm×0.7 mm, ultrasonically cleaned with isopropyl alcohol and pure water for 5 minutes each, cleaned by ultraviolet (UV) irradiation and ozone exposure for 30 minutes, and then mounted in a vacuum deposition apparatus.

On the anode, NPD was deposited to form a hole injection layer having the thickness of 300 Å, followed by deposition of HT3 on the hole injection layer to form a hole transport layer having the thickness of 200 Å, and then CzSi was deposited to form an emission auxiliary layer having the thickness of 100 Å.

On the emission auxiliary layer, the first host, the second host, the first dopant, and the second dopant were co-deposited at a weight ratio of 42:42:15:1 with reference to Table 2 below to form an emission layer having the thickness of 200 Å.

Subsequently, TSPO1 was deposited on the emission layer to form a hole-blocking layer having the thickness of 200 Å, followed by deposition of TPBI on the hole-blocking layer to form an electron transport layer having the thickness of 300 Å, then LiF was deposited on the electron transport layer to form an electron injection layer having the thickness of 10 Å, and Al was deposited on the electron injection layer to form a cathode having the thickness of 3000 Å, thereby completing the fabrication of a light-emitting device.

### Comparative Examples 2 to 6

Light-emitting devices were manufactured in substantially the same manner as in Comparative Example 1, except that each of the compounds in Table 2 was used when forming the electron transport layer.

### Examples 1 to 10

Light-emitting devices were manufactured in substantially the same manner as in Comparative Example 1, except that each of the compounds in Table 2 was used when forming the electron transport layer.

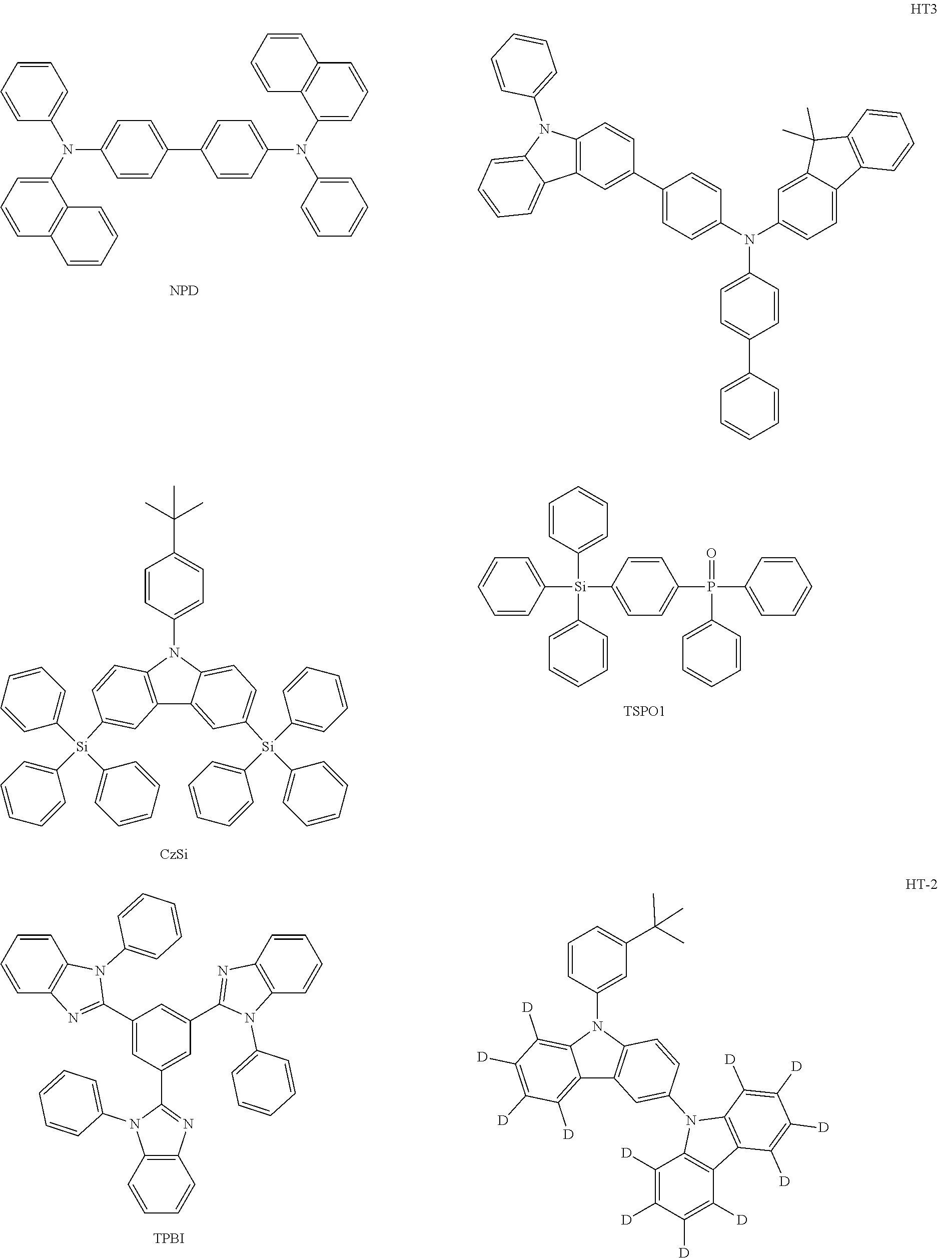

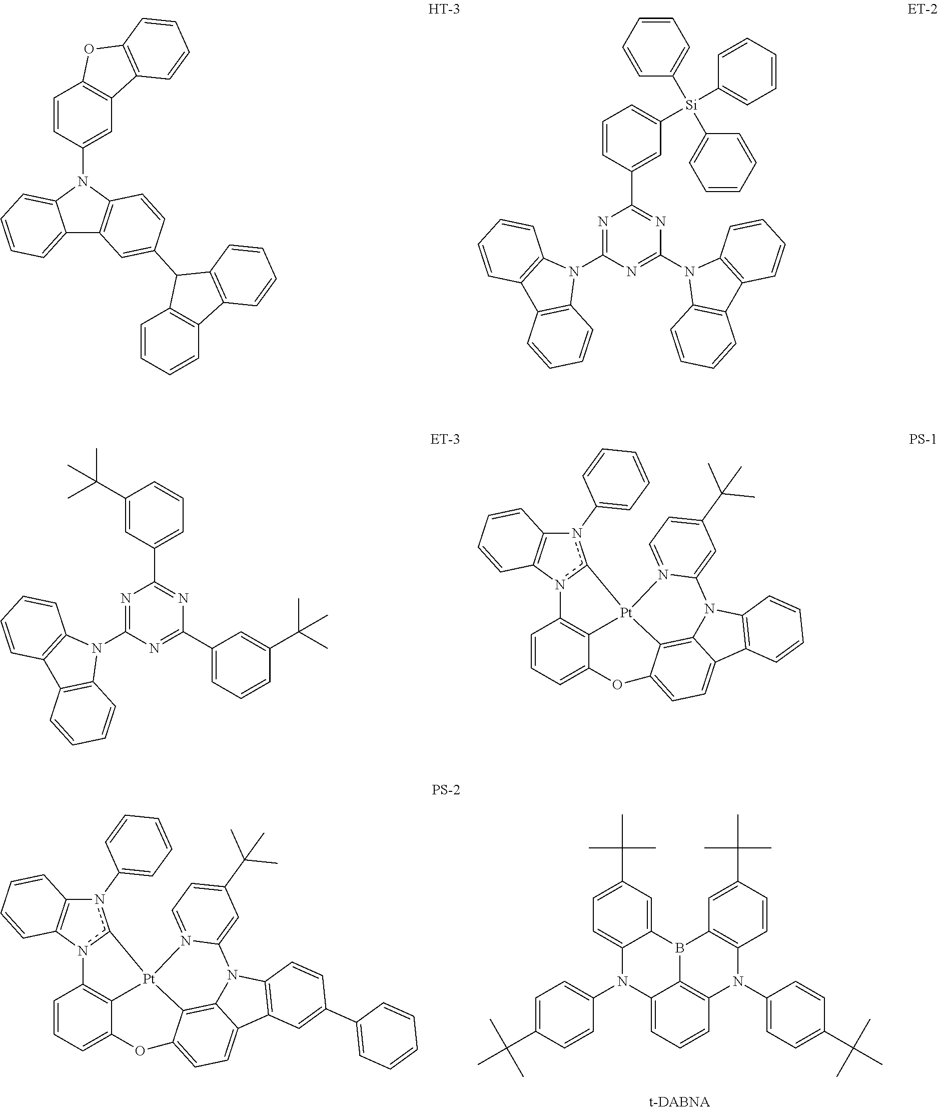

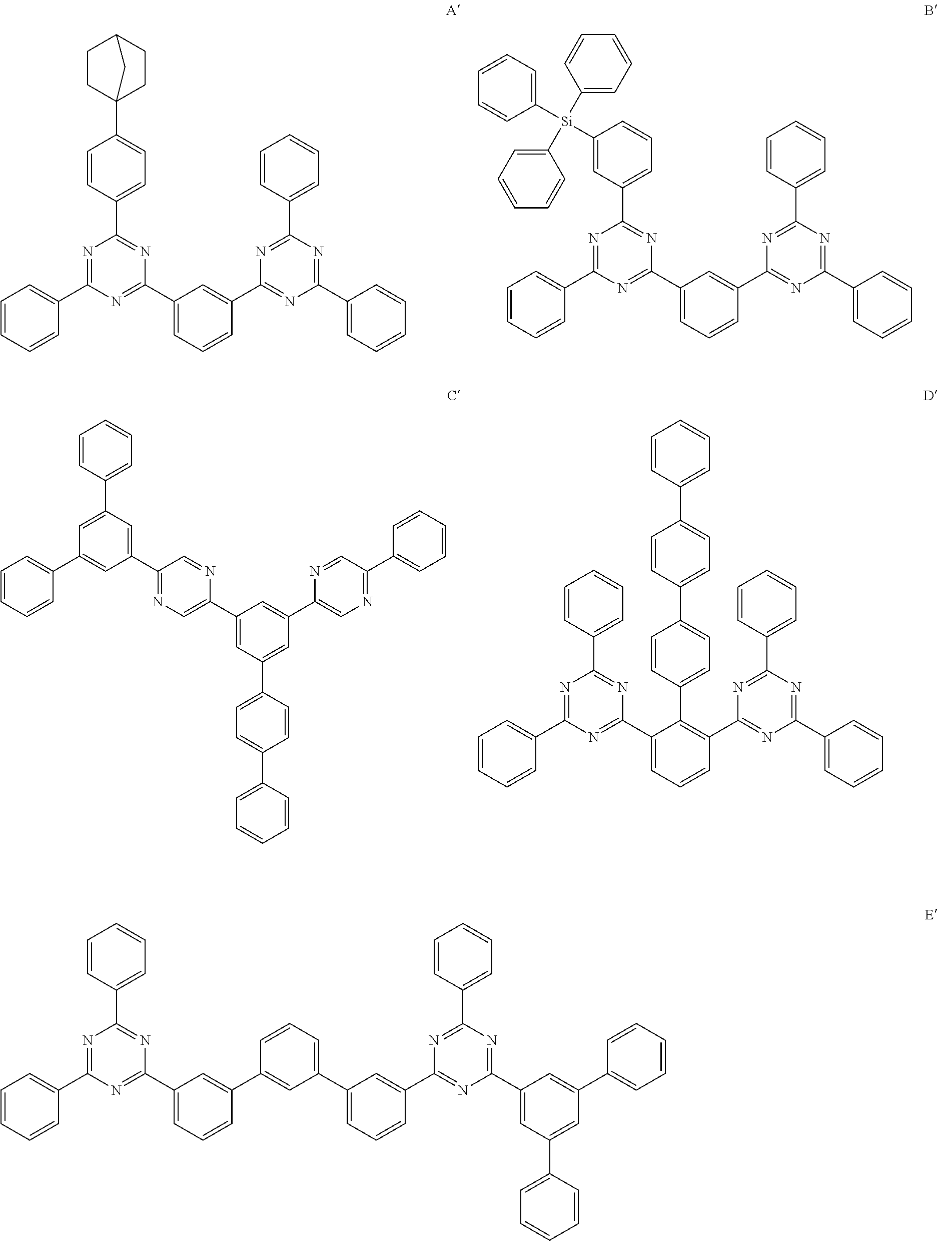

The results of the light-emitting devices are shown in Table 2.

The driving voltage of each of the light-emitting devices was measured using a source meter (Keithley Instrument, 2400 series), and efficiency and lifespan were measured using a Hamamatsu Photonics measurement device C9920-12.

TABLE 2

| | Hole | | | | Electron | Driving | | Lifespan |
|---|---|---|---|---|---|---|---|---|
| | transport | | First | Second | transport | voltage | Efficiency | ratio |
| | layer | HT/ET¹⁾ | dopant | dopant | layer | (V) | (cd/A) | (T95) |
| Comparative | HT3 | HT-3/ET-2 | PS-2 | t-DABNA | TPBI | 5.6 | 18.8 | 1 |
| Example 1 | | | | | | | | |
| Comparative | HT3 | HT-3/ET-2 | PS-2 | t-DABNA | A′ | 4.76 | 21.0 | 2.7 |
| Example 2 | | | | | | | | |
| Comparative | HT3 | HT-3/ET-2 | PS-2 | t-DABNA | B′ | 5.84 | 17.1 | 0.9 |
| Example 3 | | | | | | | | |
| Comparative | HT3 | HT-3/ET-2 | PS-2 | t-DABNA | C′ | 5.65 | 17.0 | 0.95 |
| Example 4 | | | | | | | | |
| Comparative | HT3 | HT-3/ET-2 | PS-2 | t-DABNA | D′ | 4.68 | 20.5 | 2.3 |
| Example 5 | | | | | | | | |
| Comparative | HT3 | HT-3/ET-2 | PS-2 | t-DABNA | E′ | 4.55 | 19.5 | 1.5 |
| Example 6 | | | | | | | | |
| Example 1 | HT3 | HT-3/ET-2 | PS-2 | t-DABNA | Compound 1 | 4.39 | 25.0 | 5.7 |
| Example 2 | HT3 | HT-3/ET-2 | PS-2 | t-DABNA | Compound 6 | 4.21 | 25.8 | 6.1 |
| Example 3 | HT3 | HT-3/ET-2 | PS-2 | t-DABNA | Compound 7 | 4.13 | 26.2 | 6.3 |
| Example 4 | HT3 | HT-3/ET-2 | PS-2 | t-DABNA | Compound 9 | 4.11 | 26.4 | 6.4 |
| Example 5 | HT3 | HT-3/ET-2 | PS-2 | t-DABNA | Compound 12 | 4.44 | 24.7 | 5.5 |
| Example 6 | HT3 | HT-2/ET-3 | PS-1 | t-DABNA | Compound 14 | 4.12 | 26.3 | 6.3 |
| Example 7 | HT3 | HT-2/ET-3 | PS-1 | t-DABNA | Compound 15 | 4.11 | 26.3 | 6.4 |
| Example 8 | HT3 | HT-2/ET-3 | PS-1 | t-DABNA | Compound 181 | 4.47 | 24.6 | 5.5 |
| Example 9 | HT3 | HT-2/ET-3 | PS-1 | t-DABNA | Compound 186 | 4.30 | 25.4 | 5.9 |
| Example 10 | HT3 | HT-2/ET-3 | PS-1 | t-DABNA | Compound 192 | 4.53 | 24.3 | 5.3 |

¹⁾HT: hole-transporting host
ET: electron-transporting host
HT:ET = 5:5 (weight ratio)

Referring to Table 2, it can be seen that the driving voltage, efficiency, and lifespan of Examples are superior to those of the Comparative Examples.

Light-emitting devices according to embodiments are superior to light-emitting devices of the related art.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various suitable changes in form and details may be made therein without departing from the spirit and scope as defined by the following claims, and equivalents thereof.

What is claimed is:

1. A compound represented by Formula 1: 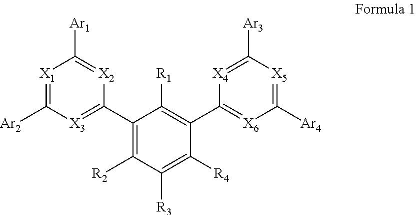
wherein, in Formula 1,
X₁ is N or CR₁₁, X₂ is N or CR₁₂, X₃ is N or CR₁₃, and at least one selected from among X₁ to X₃ is N,
X₄ is N or CR₁₄, X₅ is N or CR₁₅, X₆ is N or CR₁₆, and at least one selected from among X₄ to X₆ is N,
R₁ to R₄ and R₁₁ to R₁₆ are each independently hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₃-C₁₀ cycloalkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₁-C₆₀ alkylthio group unsubstituted or substituted with at least one R₁₀ₐ, a C₆-C₆₀ aryl group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₁-C₆₀ heteroaryl group unsubstituted or substituted with at least one R₁₀ₐ, a C₆-C₆₀ non-aromatic condensed polycyclic group unsubstituted or substituted with at least one R₁₀ₐ, a C₁-C₆₀ non-aromatic condensed heteropolycyclic group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂), Ar₁ to Ar₄ are each independently a C₆-C₆₀ aryl group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₁-C₆₀ heteroaryl group unsubstituted or substituted with at least one R₁₀ₐ, a C₈-C₆₀ non-aromatic condensed polycyclic group unsubstituted or substituted with at least one R₁₀ₐ, or a C₁-C₆₀ non-aromatic condensed heteropolycyclic group unsubstituted or substituted with at least one R₁₀ₐ,
at least one selected from among Ar₁ to Ar₄ is an unsubstituted terphenyl group,
R₁₀ₐ is:
hydrogen, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ are each independently hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, or a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof.

2. The compound of claim 1, wherein:
the unsubstituted terphenyl group is represented by Formula Y1 or Formula Y2: 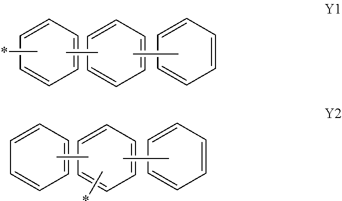
wherein * in Formulae Y1 and Y2 indicates a binding site to a neighboring atom.

3. The compound of claim 1, wherein:
the unsubstituted terphenyl group is represented by one of Formula Y11 to Formula Y25: 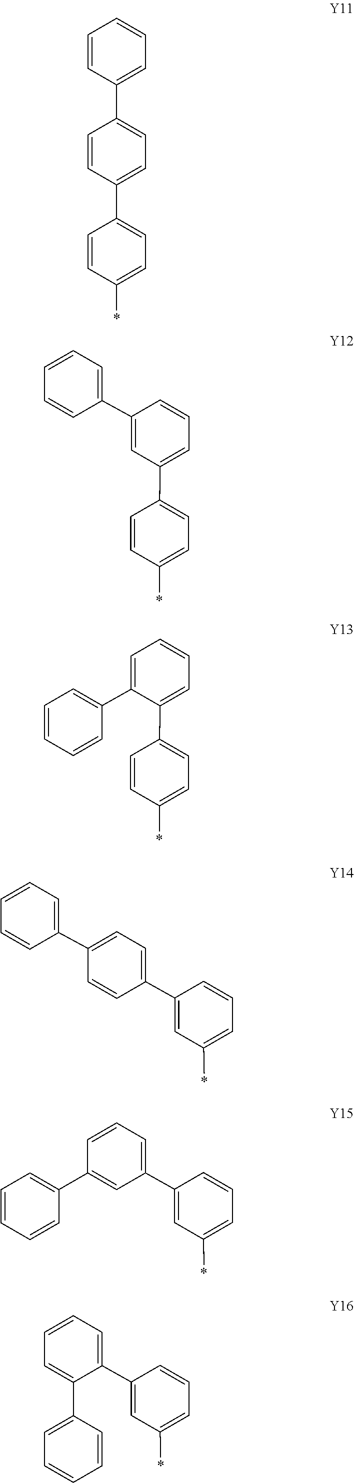 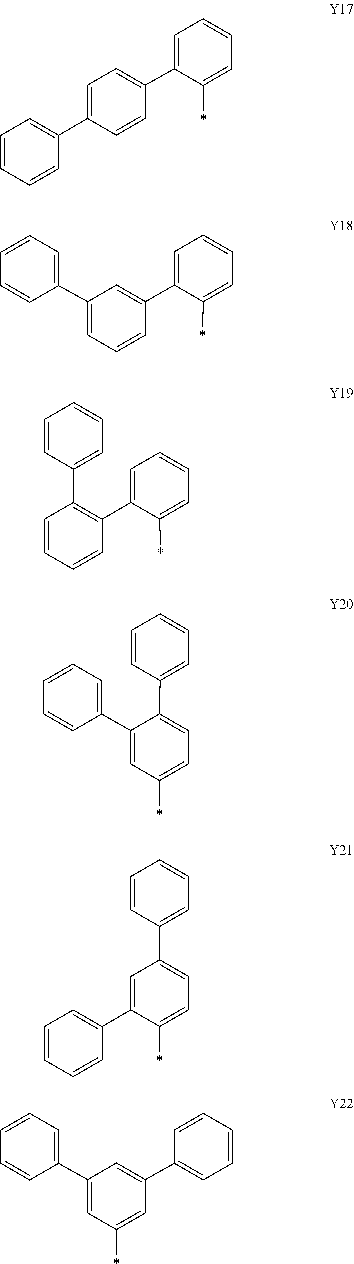 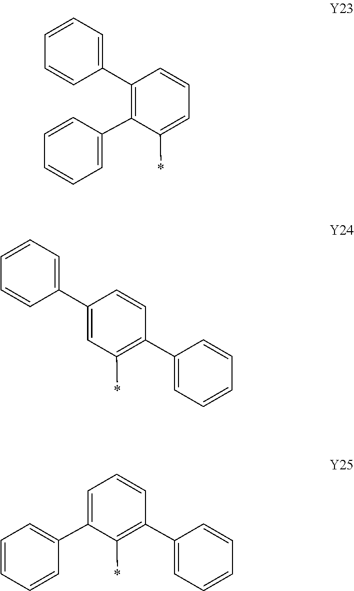 [SMILES: *c1cccc(-c2ccccc2)c1-c1ccccc1 ; *c1cc(-c2ccccc2)ccc1-c1ccccc1 ; *c1c(-c2ccccc2)cccc1-c1ccccc1]
wherein * in Formulae Y11 to Y25 indicates a binding site to a neighboring atom.

4. The compound of claim 1, wherein:
substituents other than the unsubstituted terphenyl groups of Ar₁ to Ar₄ are each independently a C₆-C₆₀ aryl group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, or a C₁-C₆₀ heteroaryl group unsubstituted or substituted with at least one R₁₀ₐ.

5. The compound of claim 1, wherein:
Ar₁ or Ar₃ is an unsubstituted terphenyl group.

6. The compound of claim 1, wherein:
R₁ to R₄ are each independently —F, —Cl, —Br, —I, a cyano group, or a C₁-C₆₀ alkyl group unsubstituted or substituted with at least one R₁₀ₐ.

7. The compound of claim 1, wherein:
R₁₁ and R₁₅ are each independently hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₃-C₁₀ cycloalkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₁-C₆₀ alkylthio group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₁-C₆₀ heteroaryl group unsubstituted or substituted with at least one R₁₀ₐ, a C₆-C₆₀ non-aromatic condensed polycyclic group unsubstituted or substituted with at least one R₁₀ₐ, a C₁-C₆₀ non-aromatic condensed heteropolycyclic group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂).

8. The compound of claim 1, wherein:
the compound represented by Formula 1 is one selected from among the following compounds:

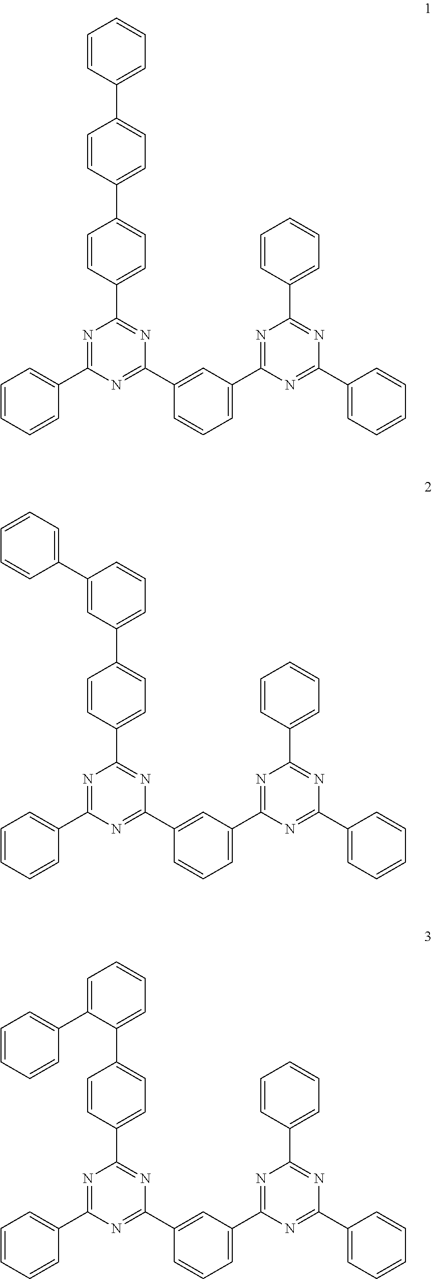 [SMILES: c1ccc(-c2ccc(-c3ccc(-c4nc(-c5ccccc5)nc(-c5cccc(-c6nc(-c7ccccc7)nc(-c7ccccc7)n6)c5)n4)cc3)cc2)cc1 ; c1ccc(-c2cccc(-c3ccc(-c4nc(-c5ccccc5)nc(-c5cccc(-c6nc(-c7ccccc7)nc(-c7ccccc7)n6)c5)n4)cc3)c2)cc1 ; c1ccc(-c2nc(-c3ccccc3)nc(-c3cccc(-c4nc(-c5ccccc5)nc(-c5ccc(-c6ccccc6-c6ccccc6)cc5)n4)c3)n2)cc1]

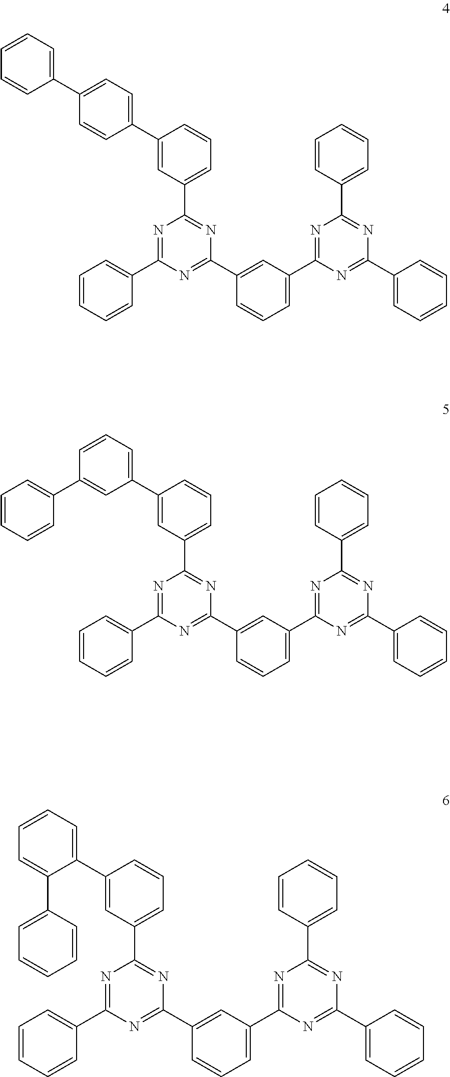 [SMILES: c1ccc(-c2ccc(-c3cccc(-c4nc(-c5ccccc5)nc(-c5cccc(-c6nc(-c7ccccc7)nc(-c7ccccc7)n6)c5)n4)c3)cc2)cc1 ; c1ccc(-c2cccc(-c3cccc(-c4nc(-c5ccccc5)nc(-c5cccc(-c6nc(-c7ccccc7)nc(-c7ccccc7)n6)c5)n4)c3)c2)cc1 ; c1ccc(-c2nc(-c3ccccc3)nc(-c3cccc(-c4nc(-c5ccccc5)nc(-c5cccc(-c6ccccc6-c6ccccc6)c5)n4)c3)n2)cc1]

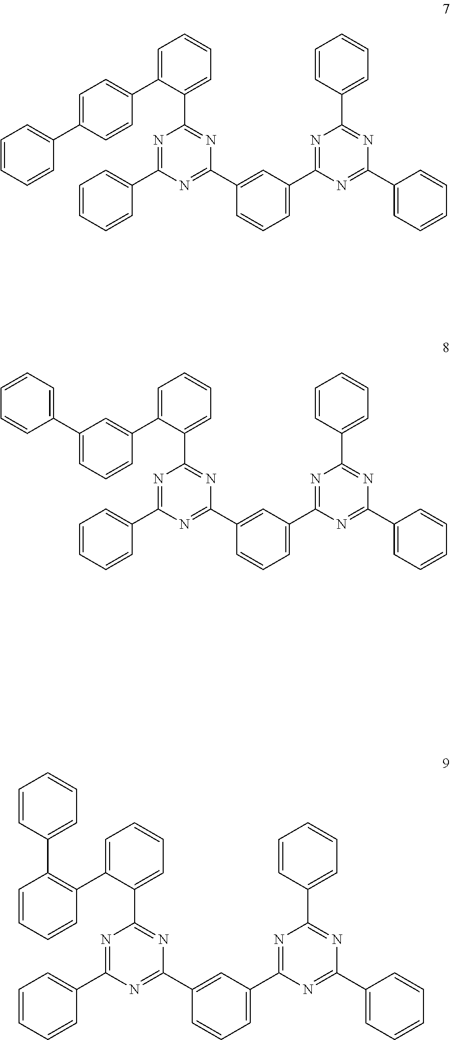 [SMILES: c1ccc(-c2ccc(-c3ccccc3-c3nc(-c4ccccc4)nc(-c4cccc(-c5nc(-c6ccccc6)nc(-c6ccccc6)n5)c4)n3)cc2)cc1 ; c1ccc(-c2cccc(-c3ccccc3-c3nc(-c4ccccc4)nc(-c4cccc(-c5nc(-c6ccccc6)nc(-c6ccccc6)n5)c4)n3)c2)cc1 ; c1ccc(-c2nc(-c3ccccc3)nc(-c3cccc(-c4nc(-c5ccccc5)nc(-c5ccccc5-c5ccccc5-c5ccccc5)n4)c3)n2)cc1]

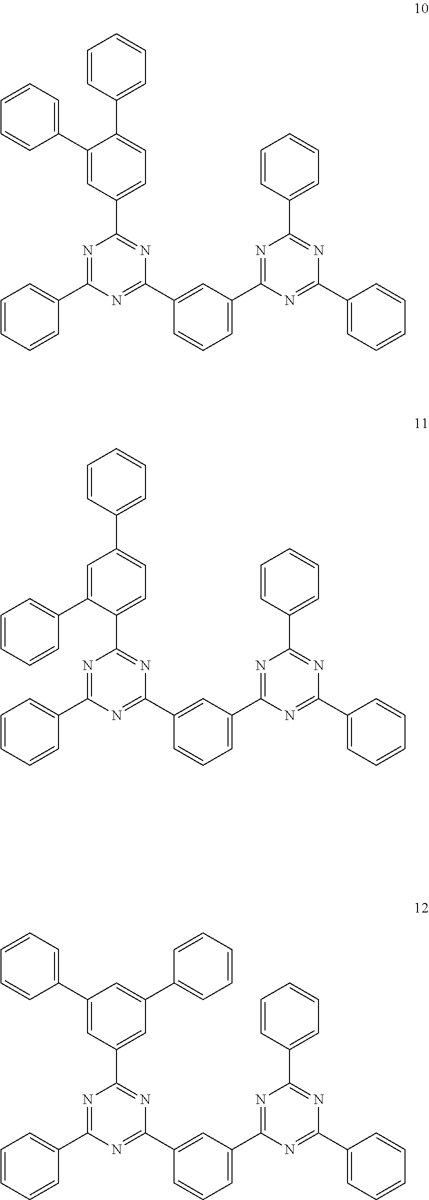 [SMILES: c1ccc(-c2nc(-c3ccccc3)nc(-c3cccc(-c4nc(-c5ccccc5)nc(-c5ccc(-c6ccccc6)c(-c6ccccc6)c5)n4)c3)n2)cc1 ; c1ccc(-c2ccc(-c3nc(-c4ccccc4)nc(-c4cccc(-c5nc(-c6ccccc6)nc(-c6ccccc6)n5)c4)n3)c(-c3ccccc3)c2)cc1 ; c1ccc(-c2cc(-c3ccccc3)cc(-c3nc(-c4ccccc4)nc(-c4cccc(-c5nc(-c6ccccc6)nc(-c6ccccc6)n5)c4)n3)c2)cc1]

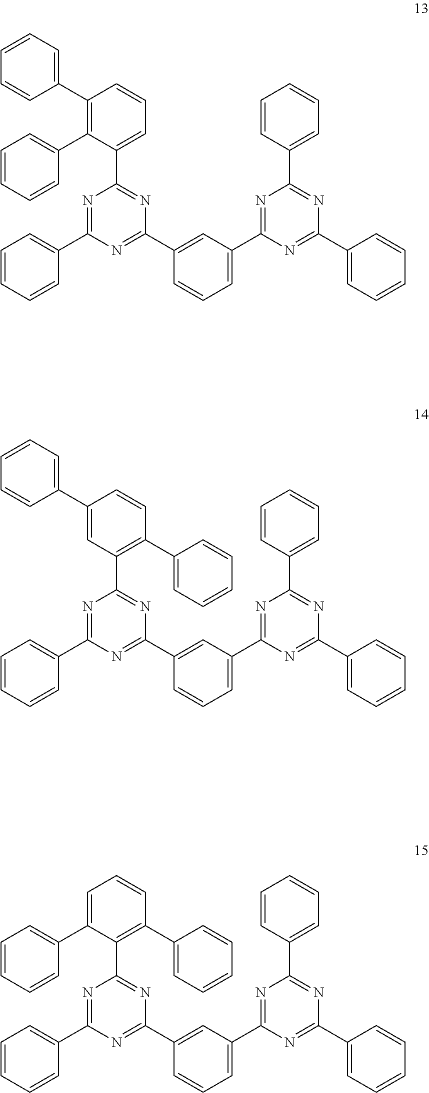 [SMILES: c1ccc(-c2nc(-c3ccccc3)nc(-c3cccc(-c4nc(-c5ccccc5)nc(-c5cccc(-c6ccccc6)c5-c5ccccc5)n4)c3)n2)cc1 ; c1ccc(-c2ccc(-c3ccccc3)c(-c3nc(-c4ccccc4)nc(-c4cccc(-c5nc(-c6ccccc6)nc(-c6ccccc6)n5)c4)n3)c2)cc1 ; c1ccc(-c2nc(-c3ccccc3)nc(-c3cccc(-c4nc(-c5ccccc5)nc(-c5c(-c6ccccc6)cccc5-c5ccccc5)n4)c3)n2)cc1]

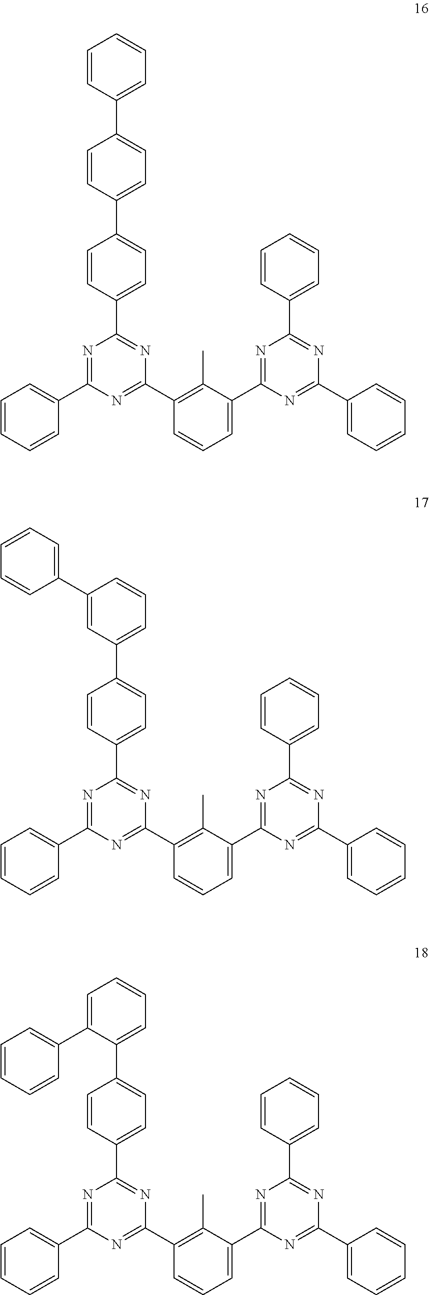 [SMILES: Cc1c(-c2nc(-c3ccccc3)nc(-c3ccccc3)n2)cccc1-c1nc(-c2ccccc2)nc(-c2ccc(-c3ccc(-c4ccccc4)cc3)cc2)n1 ; Cc1c(-c2nc(-c3ccccc3)nc(-c3ccccc3)n2)cccc1-c1nc(-c2ccccc2)nc(-c2ccc(-c3cccc(-c4ccccc4)c3)cc2)n1 ; Cc1c(-c2nc(-c3ccccc3)nc(-c3ccccc3)n2)cccc1-c1nc(-c2ccccc2)nc(-c2ccc(-c3ccccc3-c3ccccc3)cc2)n1]

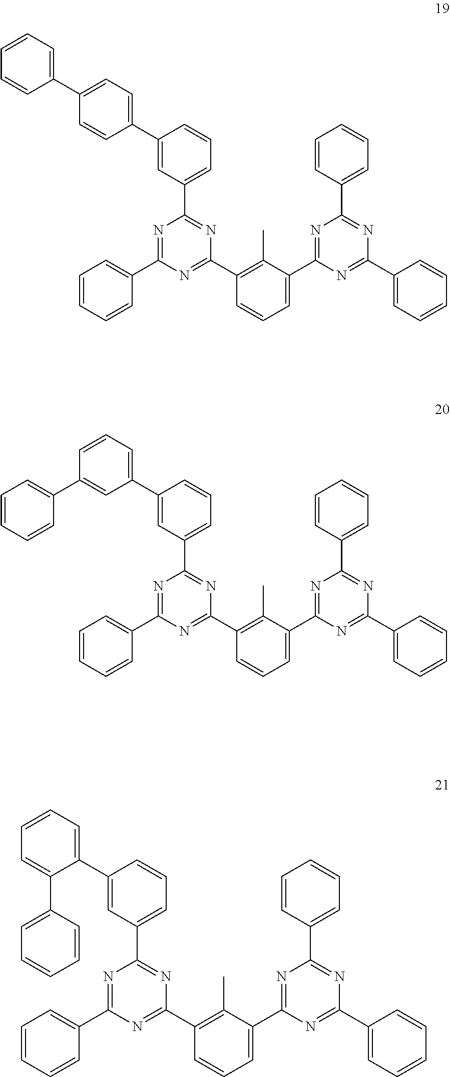 [SMILES: Cc1c(-c2nc(-c3ccccc3)nc(-c3ccccc3)n2)cccc1-c1nc(-c2ccccc2)nc(-c2cccc(-c3ccc(-c4ccccc4)cc3)c2)n1 ; Cc1c(-c2nc(-c3ccccc3)nc(-c3ccccc3)n2)cccc1-c1nc(-c2ccccc2)nc(-c2cccc(-c3cccc(-c4ccccc4)c3)c2)n1 ; Cc1c(-c2nc(-c3ccccc3)nc(-c3ccccc3)n2)cccc1-c1nc(-c2ccccc2)nc(-c2cccc(-c3ccccc3-c3ccccc3)c2)n1]

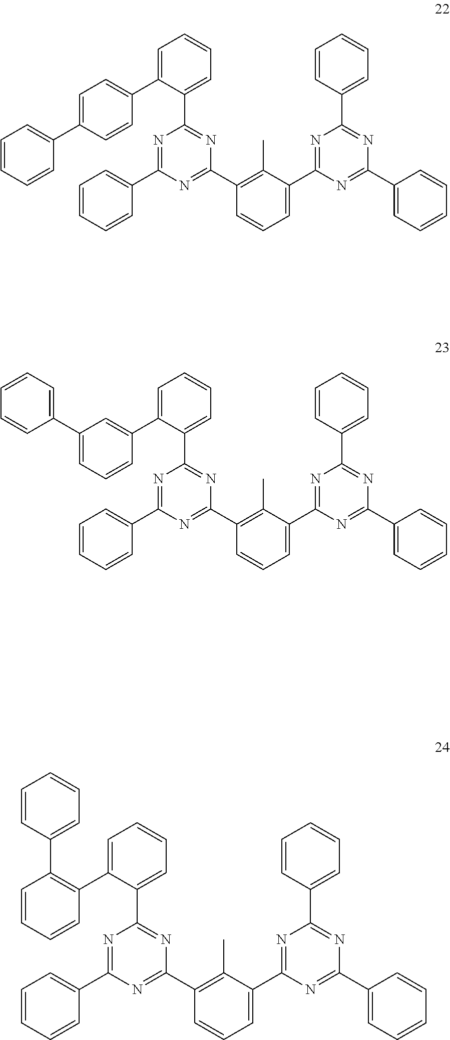 [SMILES: Cc1c(-c2nc(-c3ccccc3)nc(-c3ccccc3)n2)cccc1-c1nc(-c2ccccc2)nc(-c2ccccc2-c2ccc(-c3ccccc3)cc2)n1 ; Cc1c(-c2nc(-c3ccccc3)nc(-c3ccccc3)n2)cccc1-c1nc(-c2ccccc2)nc(-c2ccccc2-c2cccc(-c3ccccc3)c2)n1 ; Cc1c(-c2nc(-c3ccccc3)nc(-c3ccccc3)n2)cccc1-c1nc(-c2ccccc2)nc(-c2ccccc2-c2ccccc2-c2ccccc2)n1]

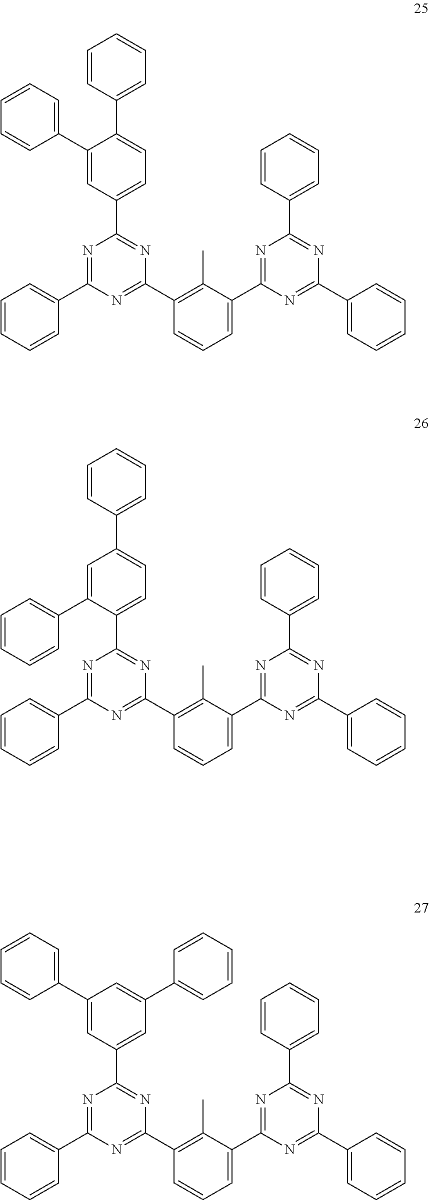 [SMILES: Cc1c(-c2nc(-c3ccccc3)nc(-c3ccccc3)n2)cccc1-c1nc(-c2ccccc2)nc(-c2ccc(-c3ccccc3)c(-c3ccccc3)c2)n1 ; Cc1c(-c2nc(-c3ccccc3)nc(-c3ccccc3)n2)cccc1-c1nc(-c2ccccc2)nc(-c2ccc(-c3ccccc3)cc2-c2ccccc2)n1 ; Cc1c(-c2nc(-c3ccccc3)nc(-c3ccccc3)n2)cccc1-c1nc(-c2ccccc2)nc(-c2cc(-c3ccccc3)cc(-c3ccccc3)c2)n1]

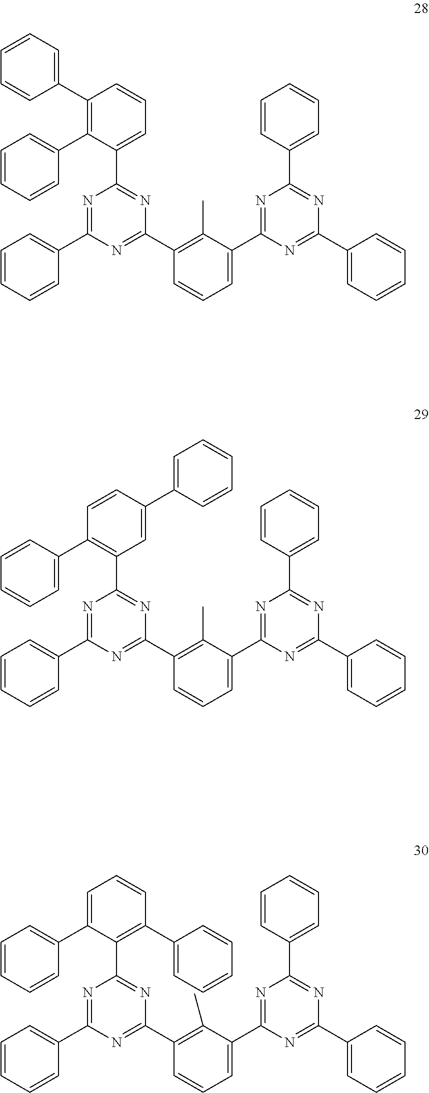 [SMILES: Cc1c(-c2nc(-c3ccccc3)nc(-c3ccccc3)n2)cccc1-c1nc(-c2ccccc2)nc(-c2cccc(-c3ccccc3)c2-c2ccccc2)n1 ; Cc1c(-c2nc(-c3ccccc3)nc(-c3ccccc3)n2)cccc1-c1nc(-c2ccccc2)nc(-c2cc(-c3ccccc3)ccc2-c2ccccc2)n1 ; Cc1c(-c2nc(-c3ccccc3)nc(-c3ccccc3)n2)cccc1-c1nc(-c2ccccc2)nc(-c2c(-c3ccccc3)cccc2-c2ccccc2)n1]

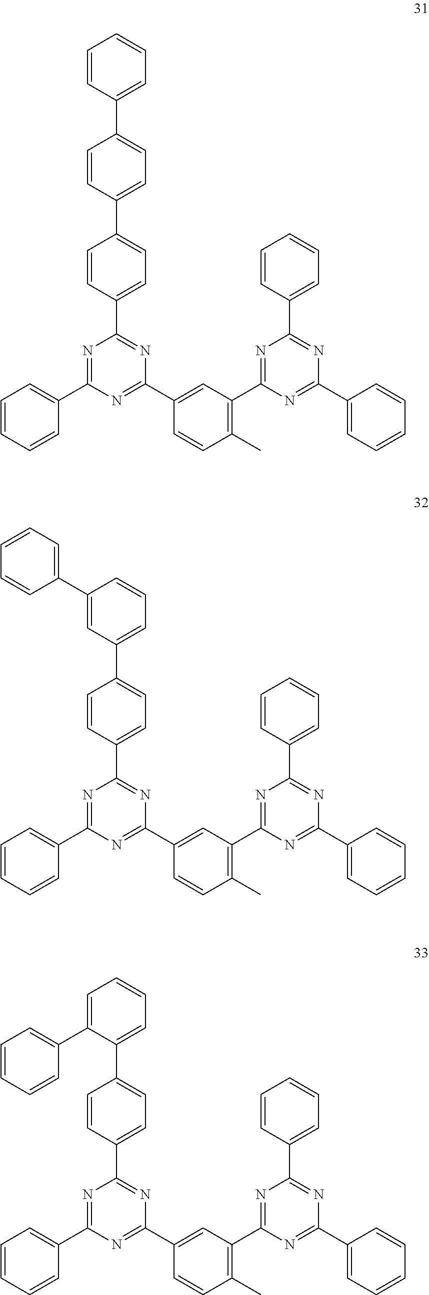 [SMILES: Cc1ccc(-c2nc(-c3ccccc3)nc(-c3ccc(-c4ccc(-c5ccccc5)cc4)cc3)n2)cc1-c1nc(-c2ccccc2)nc(-c2ccccc2)n1 ; Cc1ccc(-c2nc(-c3ccccc3)nc(-c3ccc(-c4cccc(-c5ccccc5)c4)cc3)n2)cc1-c1nc(-c2ccccc2)nc(-c2ccccc2)n1 ; Cc1ccc(-c2nc(-c3ccccc3)nc(-c3ccc(-c4ccccc4-c4ccccc4)cc3)n2)cc1-c1nc(-c2ccccc2)nc(-c2ccccc2)n1]

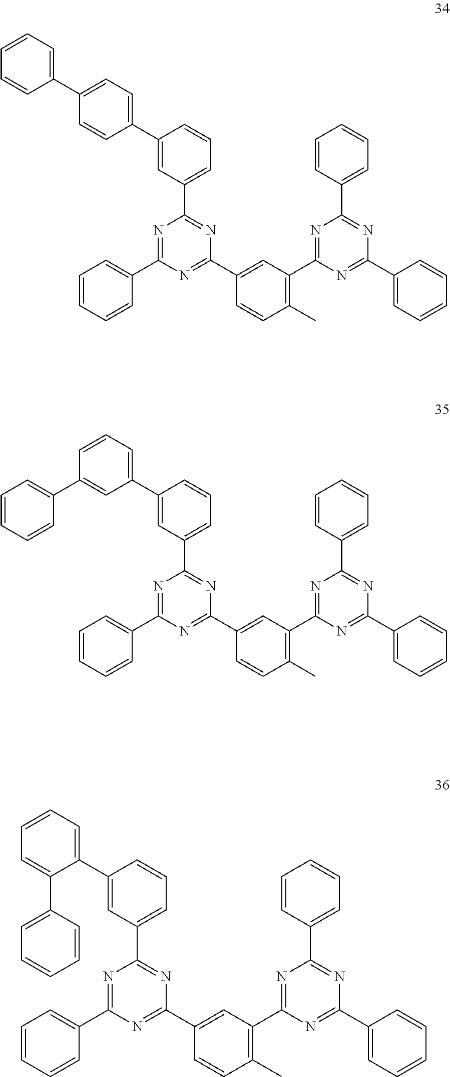 [SMILES: Cc1ccc(-c2nc(-c3ccccc3)nc(-c3cccc(-c4ccc(-c5ccccc5)cc4)c3)n2)cc1-c1nc(-c2ccccc2)nc(-c2ccccc2)n1 ; Cc1ccc(-c2nc(-c3ccccc3)nc(-c3cccc(-c4cccc(-c5ccccc5)c4)c3)n2)cc1-c1nc(-c2ccccc2)nc(-c2ccccc2)n1 ; Cc1ccc(-c2nc(-c3ccccc3)nc(-c3cccc(-c4ccccc4-c4ccccc4)c3)n2)cc1-c1nc(-c2ccccc2)nc(-c2ccccc2)n1]

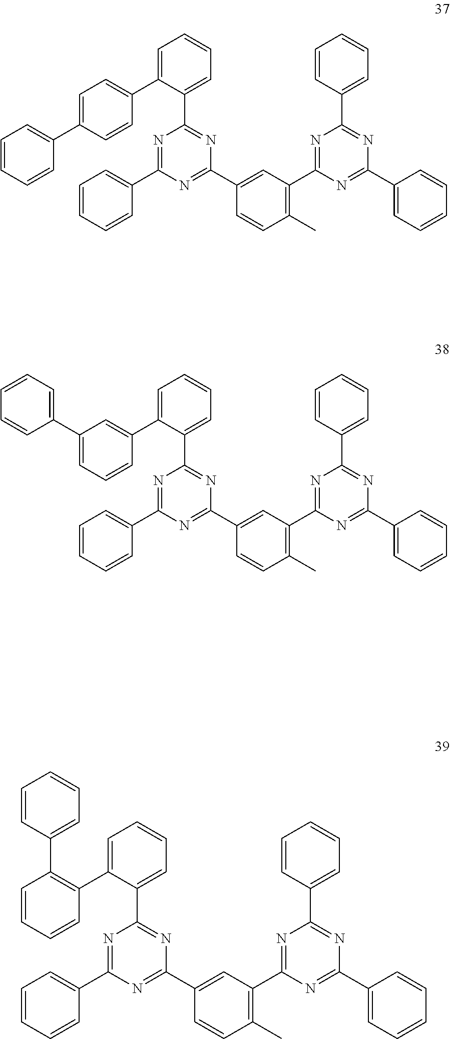 [SMILES: Cc1ccc(-c2nc(-c3ccccc3)nc(-c3ccccc3-c3ccc(-c4ccccc4)cc3)n2)cc1-c1nc(-c2ccccc2)nc(-c2ccccc2)n1 ; Cc1ccc(-c2nc(-c3ccccc3)nc(-c3ccccc3-c3cccc(-c4ccccc4)c3)n2)cc1-c1nc(-c2ccccc2)nc(-c2ccccc2)n1 ; Cc1ccc(-c2nc(-c3ccccc3)nc(-c3ccccc3-c3ccccc3-c3ccccc3)n2)cc1-c1nc(-c2ccccc2)nc(-c2ccccc2)n1]

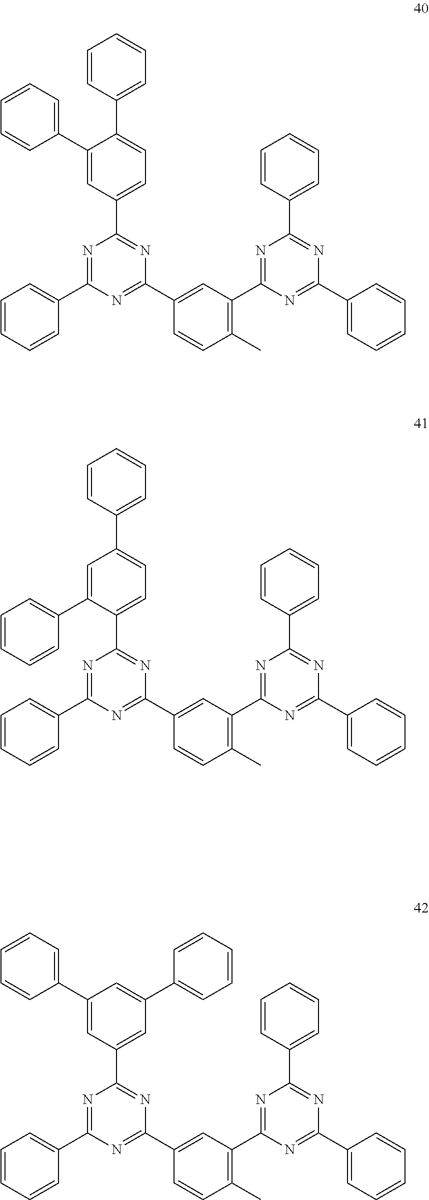 [SMILES: Cc1ccc(-c2nc(-c3ccccc3)nc(-c3ccc(-c4ccccc4)c(-c4ccccc4)c3)n2)cc1-c1nc(-c2ccccc2)nc(-c2ccccc2)n1 ; Cc1ccc(-c2nc(-c3ccccc3)nc(-c3ccc(-c4ccccc4)cc3-c3ccccc3)n2)cc1-c1nc(-c2ccccc2)nc(-c2ccccc2)n1 ; Cc1ccc(-c2nc(-c3ccccc3)nc(-c3cc(-c4ccccc4)cc(-c4ccccc4)c3)n2)cc1-c1nc(-c2ccccc2)nc(-c2ccccc2)n1]

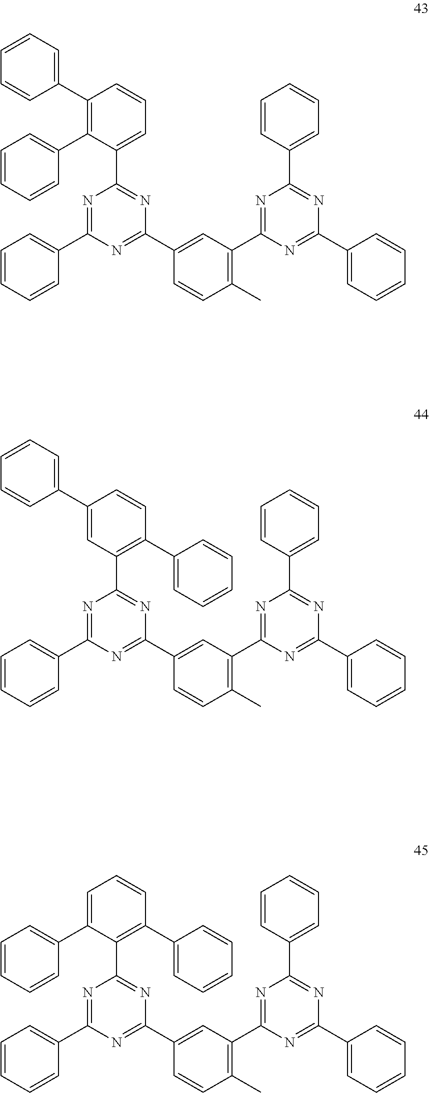 [SMILES: Cc1ccc(-c2nc(-c3ccccc3)nc(-c3cccc(-c4ccccc4)c3-c3ccccc3)n2)cc1-c1nc(-c2ccccc2)nc(-c2ccccc2)n1 ; Cc1ccc(-c2nc(-c3ccccc3)nc(-c3cc(-c4ccccc4)ccc3-c3ccccc3)n2)cc1-c1nc(-c2ccccc2)nc(-c2ccccc2)n1 ; Cc1ccc(-c2nc(-c3ccccc3)nc(-c3c(-c4ccccc4)cccc3-c3ccccc3)n2)cc1-c1nc(-c2ccccc2)nc(-c2ccccc2)n1]

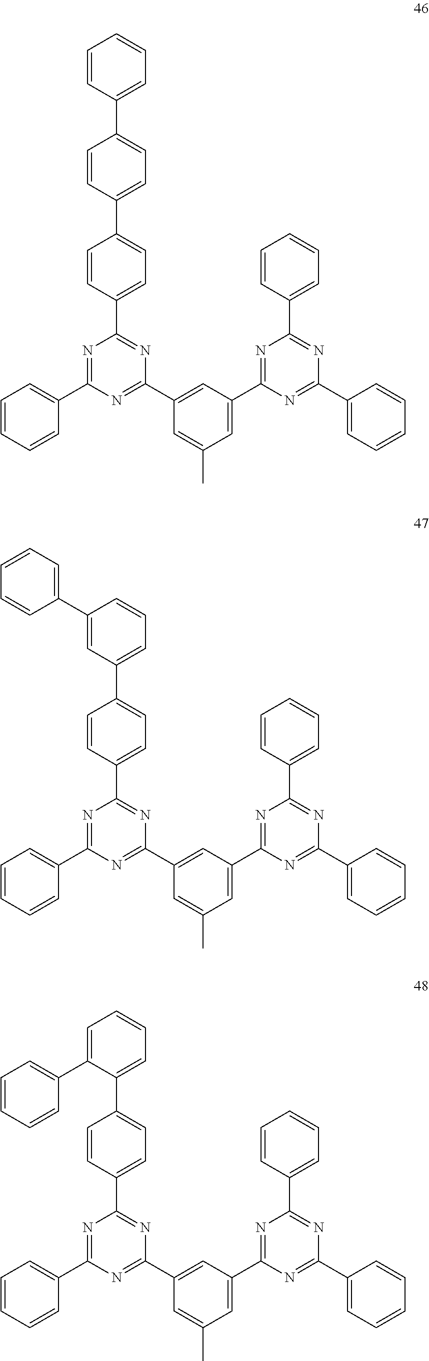 [SMILES: Cc1cc(-c2nc(-c3ccccc3)nc(-c3ccccc3)n2)cc(-c2nc(-c3ccccc3)nc(-c3ccc(-c4ccc(-c5ccccc5)cc4)cc3)n2)c1 ; Cc1cc(-c2nc(-c3ccccc3)nc(-c3ccccc3)n2)cc(-c2nc(-c3ccccc3)nc(-c3ccc(-c4cccc(-c5ccccc5)c4)cc3)n2)c1 ; Cc1cc(-c2nc(-c3ccccc3)nc(-c3ccccc3)n2)cc(-c2nc(-c3ccccc3)nc(-c3ccc(-c4ccccc4-c4ccccc4)cc3)n2)c1]

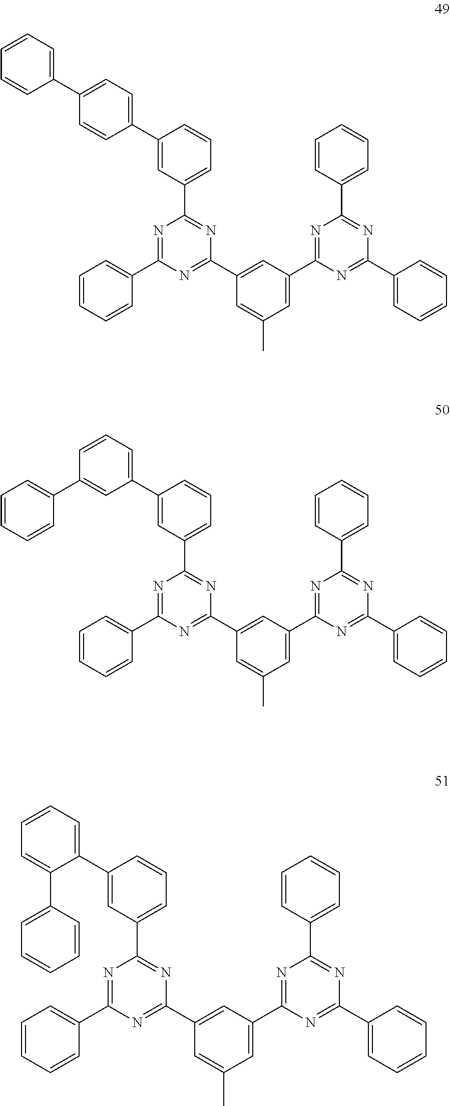 [SMILES: Cc1cc(-c2nc(-c3ccccc3)nc(-c3ccccc3)n2)cc(-c2nc(-c3ccccc3)nc(-c3cccc(-c4ccc(-c5ccccc5)cc4)c3)n2)c1 ; Cc1cc(-c2nc(-c3ccccc3)nc(-c3ccccc3)n2)cc(-c2nc(-c3ccccc3)nc(-c3cccc(-c4cccc(-c5ccccc5)c4)c3)n2)c1 ; Cc1cc(-c2nc(-c3ccccc3)nc(-c3ccccc3)n2)cc(-c2nc(-c3ccccc3)nc(-c3cccc(-c4ccccc4-c4ccccc4)c3)n2)c1]

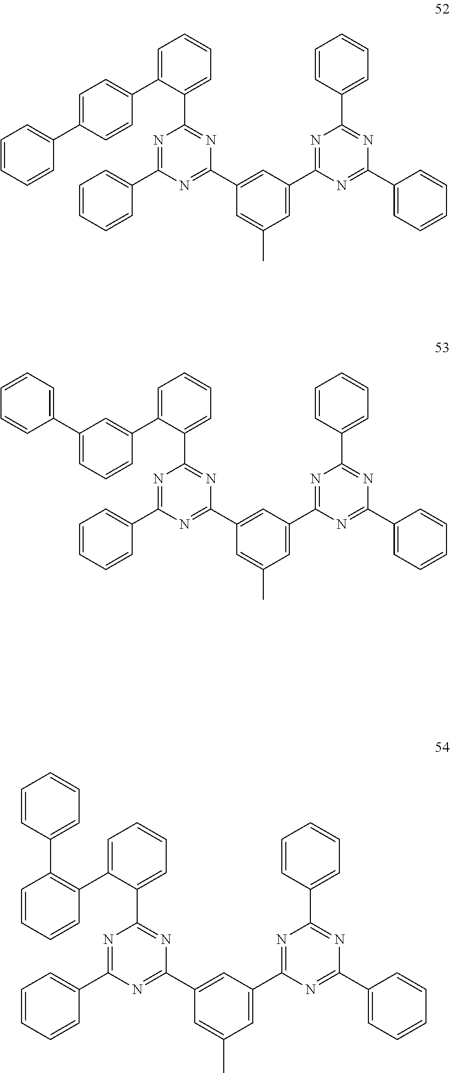 [SMILES: Cc1cc(-c2nc(-c3ccccc3)nc(-c3ccccc3)n2)cc(-c2nc(-c3ccccc3)nc(-c3ccccc3-c3ccc(-c4ccccc4)cc3)n2)c1 ; Cc1cc(-c2nc(-c3ccccc3)nc(-c3ccccc3)n2)cc(-c2nc(-c3ccccc3)nc(-c3ccccc3-c3cccc(-c4ccccc4)c3)n2)c1 ; Cc1cc(-c2nc(-c3ccccc3)nc(-c3ccccc3)n2)cc(-c2nc(-c3ccccc3)nc(-c3ccccc3-c3ccccc3-c3ccccc3)n2)c1]

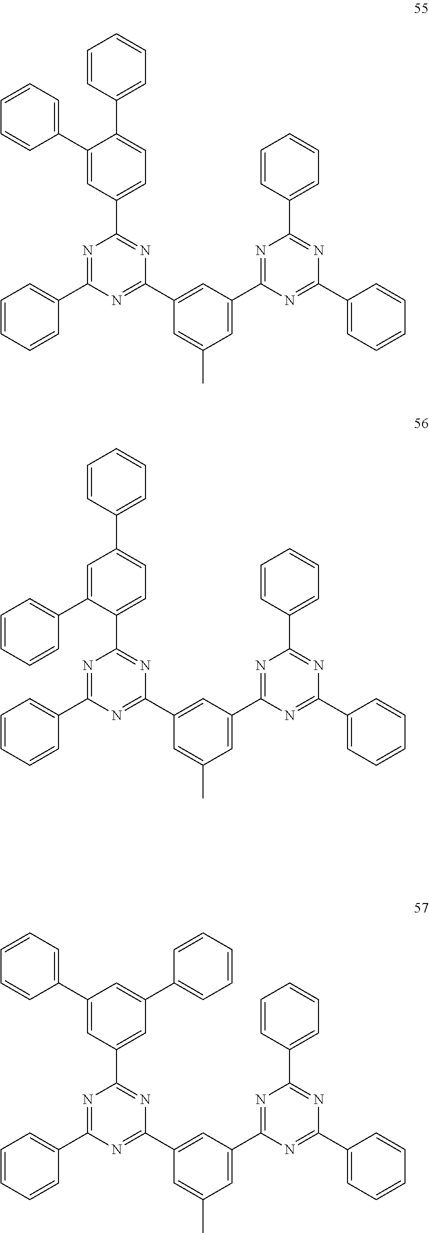 [SMILES: Cc1cc(-c2nc(-c3ccccc3)nc(-c3ccccc3)n2)cc(-c2nc(-c3ccccc3)nc(-c3ccc(-c4ccccc4)c(-c4ccccc4)c3)n2)c1 ; Cc1cc(-c2nc(-c3ccccc3)nc(-c3ccccc3)n2)cc(-c2nc(-c3ccccc3)nc(-c3ccc(-c4ccccc4)cc3-c3ccccc3)n2)c1 ; Cc1cc(-c2nc(-c3ccccc3)nc(-c3ccccc3)n2)cc(-c2nc(-c3ccccc3)nc(-c3cc(-c4ccccc4)cc(-c4ccccc4)c3)n2)c1]

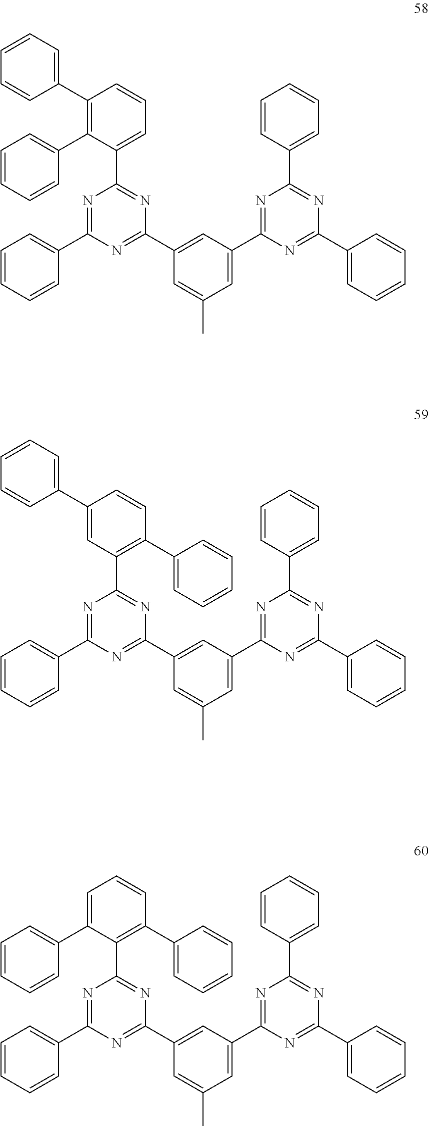 [SMILES: Cc1cc(-c2nc(-c3ccccc3)nc(-c3ccccc3)n2)cc(-c2nc(-c3ccccc3)nc(-c3cccc(-c4ccccc4)c3-c3ccccc3)n2)c1 ; Cc1cc(-c2nc(-c3ccccc3)nc(-c3ccccc3)n2)cc(-c2nc(-c3ccccc3)nc(-c3cc(-c4ccccc4)ccc3-c3ccccc3)n2)c1 ; Cc1cc(-c2nc(-c3ccccc3)nc(-c3ccccc3)n2)cc(-c2nc(-c3ccccc3)nc(-c3c(-c4ccccc4)cccc3-c3ccccc3)n2)c1]

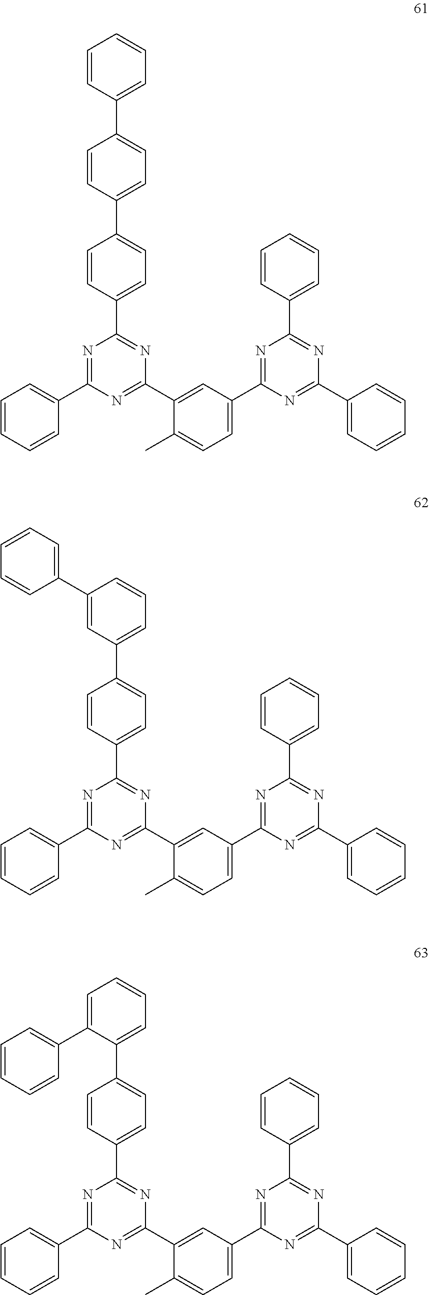 [SMILES: Cc1ccc(-c2nc(-c3ccccc3)nc(-c3ccccc3)n2)cc1-c1nc(-c2ccccc2)nc(-c2ccc(-c3ccc(-c4ccccc4)cc3)cc2)n1 ; Cc1ccc(-c2nc(-c3ccccc3)nc(-c3ccccc3)n2)cc1-c1nc(-c2ccccc2)nc(-c2ccc(-c3cccc(-c4ccccc4)c3)cc2)n1 ; Cc1ccc(-c2nc(-c3ccccc3)nc(-c3ccccc3)n2)cc1-c1nc(-c2ccccc2)nc(-c2ccc(-c3ccccc3-c3ccccc3)cc2)n1]

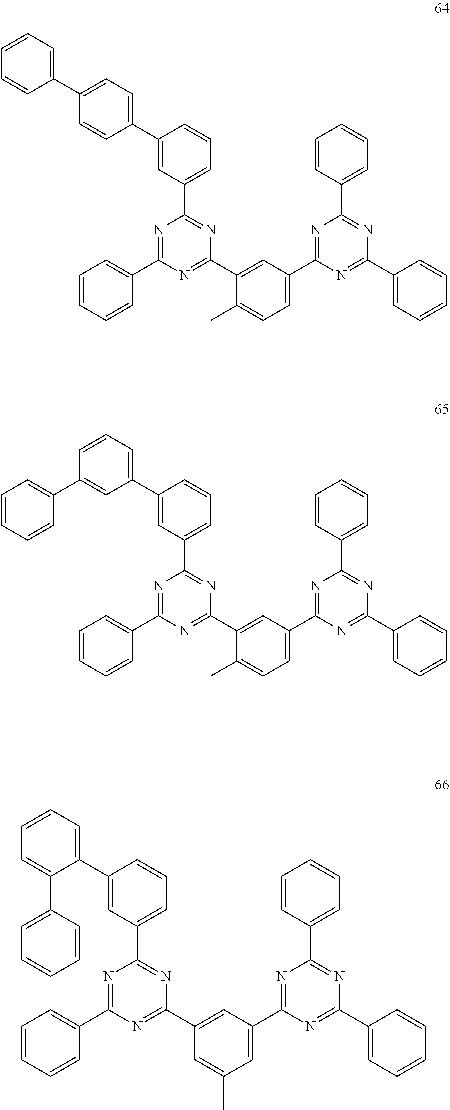 [SMILES: Cc1ccc(-c2nc(-c3ccccc3)nc(-c3ccccc3)n2)cc1-c1nc(-c2ccccc2)nc(-c2cccc(-c3ccc(-c4ccccc4)cc3)c2)n1 ; Cc1ccc(-c2nc(-c3ccccc3)nc(-c3ccccc3)n2)cc1-c1nc(-c2ccccc2)nc(-c2cccc(-c3cccc(-c4ccccc4)c3)c2)n1 ; Cc1cc(-c2nc(-c3ccccc3)nc(-c3ccccc3)n2)cc(-c2nc(-c3ccccc3)nc(-c3cccc(-c4ccccc4-c4ccccc4)c3)n2)c1]

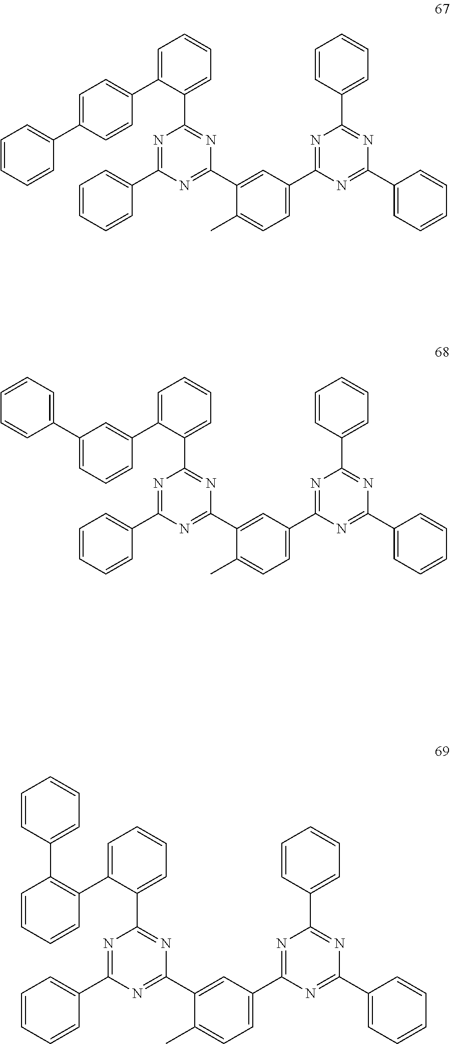 [SMILES: Cc1ccc(-c2nc(-c3ccccc3)nc(-c3ccccc3)n2)cc1-c1nc(-c2ccccc2)nc(-c2ccccc2-c2ccc(-c3ccccc3)cc2)n1 ; Cc1ccc(-c2nc(-c3ccccc3)nc(-c3ccccc3)n2)cc1-c1nc(-c2ccccc2)nc(-c2ccccc2-c2cccc(-c3ccccc3)c2)n1 ; Cc1ccc(-c2nc(-c3ccccc3)nc(-c3ccccc3)n2)cc1-c1nc(-c2ccccc2)nc(-c2ccccc2-c2ccccc2-c2ccccc2)n1]

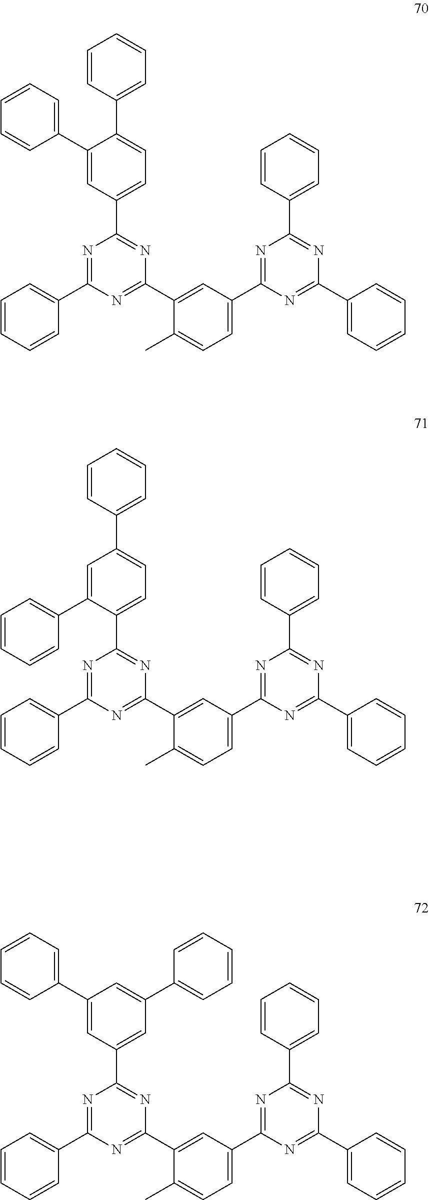 [SMILES: Cc1ccc(-c2nc(-c3ccccc3)nc(-c3ccccc3)n2)cc1-c1nc(-c2ccccc2)nc(-c2ccc(-c3ccccc3)c(-c3ccccc3)c2)n1 ; Cc1ccc(-c2nc(-c3ccccc3)nc(-c3ccccc3)n2)cc1-c1nc(-c2ccccc2)nc(-c2ccc(-c3ccccc3)cc2-c2ccccc2)n1 ; Cc1ccc(-c2nc(-c3ccccc3)nc(-c3ccccc3)n2)cc1-c1nc(-c2ccccc2)nc(-c2cc(-c3ccccc3)cc(-c3ccccc3)c2)n1]

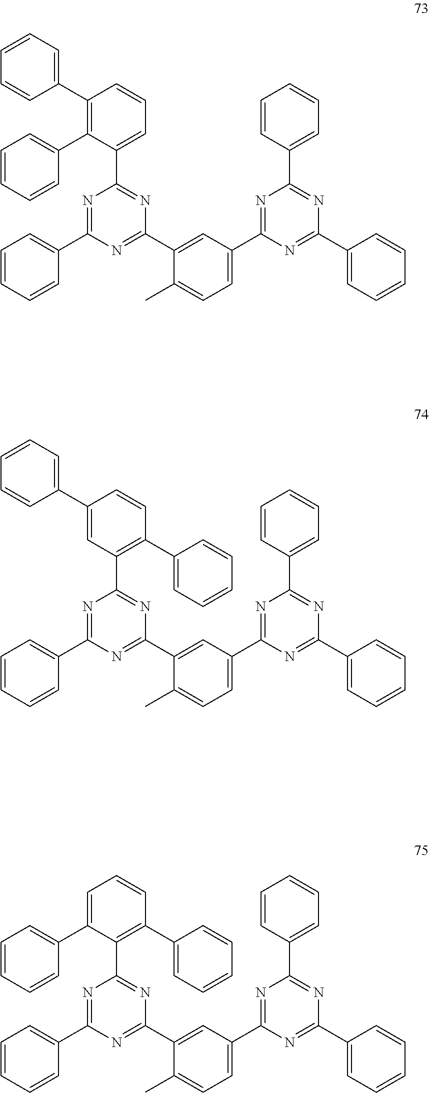 [SMILES: Cc1ccc(-c2nc(-c3ccccc3)nc(-c3ccccc3)n2)cc1-c1nc(-c2ccccc2)nc(-c2cccc(-c3ccccc3)c2-c2ccccc2)n1 ; Cc1ccc(-c2nc(-c3ccccc3)nc(-c3ccccc3)n2)cc1-c1nc(-c2ccccc2)nc(-c2cc(-c3ccccc3)ccc2-c2ccccc2)n1 ; Cc1ccc(-c2nc(-c3ccccc3)nc(-c3ccccc3)n2)cc1-c1nc(-c2ccccc2)nc(-c2c(-c3ccccc3)cccc2-c2ccccc2)n1]

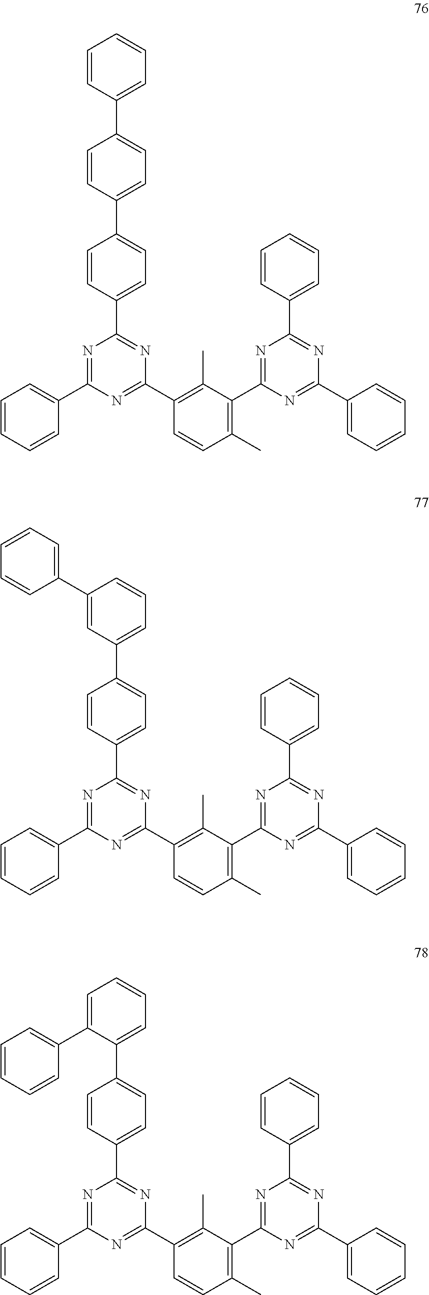 [SMILES: Cc1ccc(-c2nc(-c3ccccc3)nc(-c3ccc(-c4ccc(-c5ccccc5)cc4)cc3)n2)c(C)c1-c1nc(-c2ccccc2)nc(-c2ccccc2)n1 ; Cc1ccc(-c2nc(-c3ccccc3)nc(-c3ccc(-c4cccc(-c5ccccc5)c4)cc3)n2)c(C)c1-c1nc(-c2ccccc2)nc(-c2ccccc2)n1 ; Cc1ccc(-c2nc(-c3ccccc3)nc(-c3ccc(-c4ccccc4-c4ccccc4)cc3)n2)c(C)c1-c1nc(-c2ccccc2)nc(-c2ccccc2)n1]

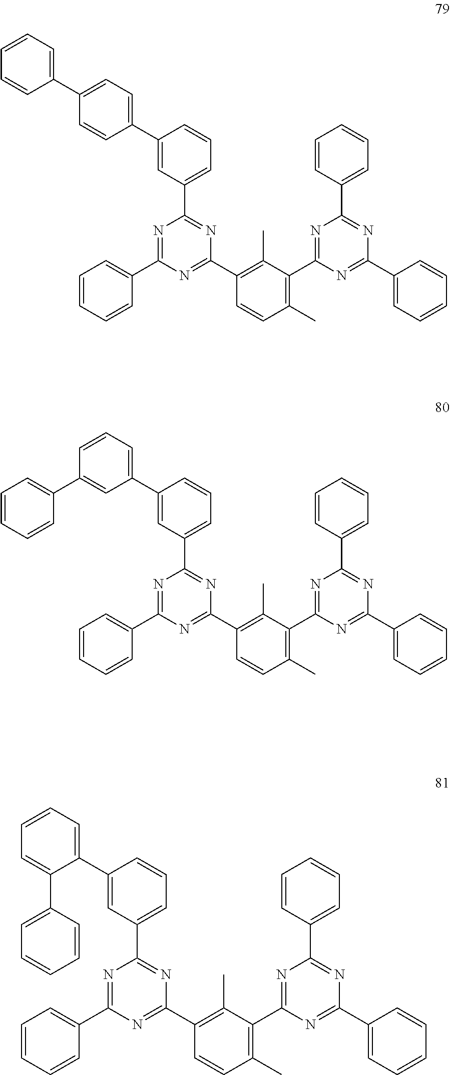 [SMILES: Cc1ccc(-c2nc(-c3ccccc3)nc(-c3cccc(-c4ccc(-c5ccccc5)cc4)c3)n2)c(C)c1-c1nc(-c2ccccc2)nc(-c2ccccc2)n1 ; Cc1ccc(-c2nc(-c3ccccc3)nc(-c3cccc(-c4cccc(-c5ccccc5)c4)c3)n2)c(C)c1-c1nc(-c2ccccc2)nc(-c2ccccc2)n1 ; Cc1ccc(-c2nc(-c3ccccc3)nc(-c3cccc(-c4ccccc4-c4ccccc4)c3)n2)c(C)c1-c1nc(-c2ccccc2)nc(-c2ccccc2)n1]

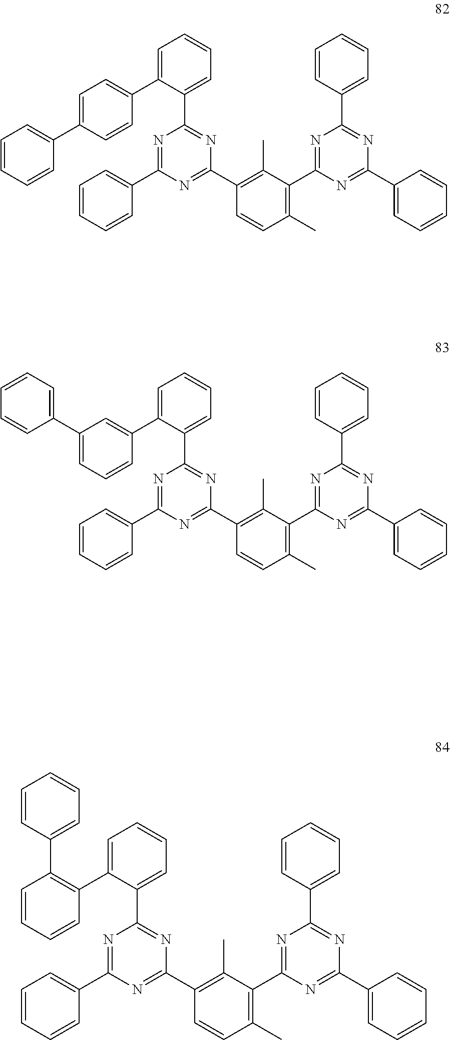 [SMILES: Cc1ccc(-c2nc(-c3ccccc3)nc(-c3ccccc3-c3ccc(-c4ccccc4)cc3)n2)c(C)c1-c1nc(-c2ccccc2)nc(-c2ccccc2)n1 ; Cc1ccc(-c2nc(-c3ccccc3)nc(-c3ccccc3-c3cccc(-c4ccccc4)c3)n2)c(C)c1-c1nc(-c2ccccc2)nc(-c2ccccc2)n1 ; Cc1ccc(-c2nc(-c3ccccc3)nc(-c3ccccc3-c3ccccc3-c3ccccc3)n2)c(C)c1-c1nc(-c2ccccc2)nc(-c2ccccc2)n1]

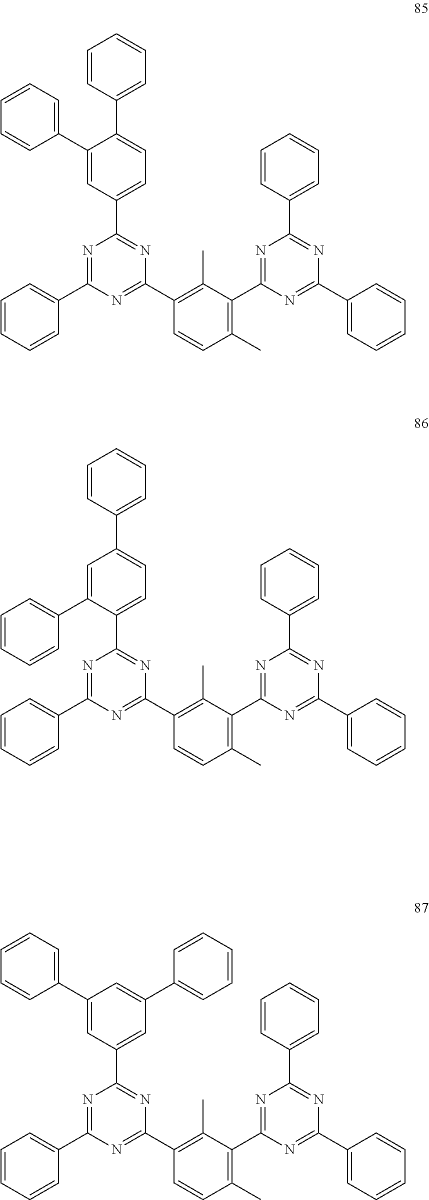 [SMILES: Cc1ccc(-c2nc(-c3ccccc3)nc(-c3ccc(-c4ccccc4)c(-c4ccccc4)c3)n2)c(C)c1-c1nc(-c2ccccc2)nc(-c2ccccc2)n1 ; Cc1ccc(-c2nc(-c3ccccc3)nc(-c3ccc(-c4ccccc4)cc3-c3ccccc3)n2)c(C)c1-c1nc(-c2ccccc2)nc(-c2ccccc2)n1 ; Cc1ccc(-c2nc(-c3ccccc3)nc(-c3cc(-c4ccccc4)cc(-c4ccccc4)c3)n2)c(C)c1-c1nc(-c2ccccc2)nc(-c2ccccc2)n1]

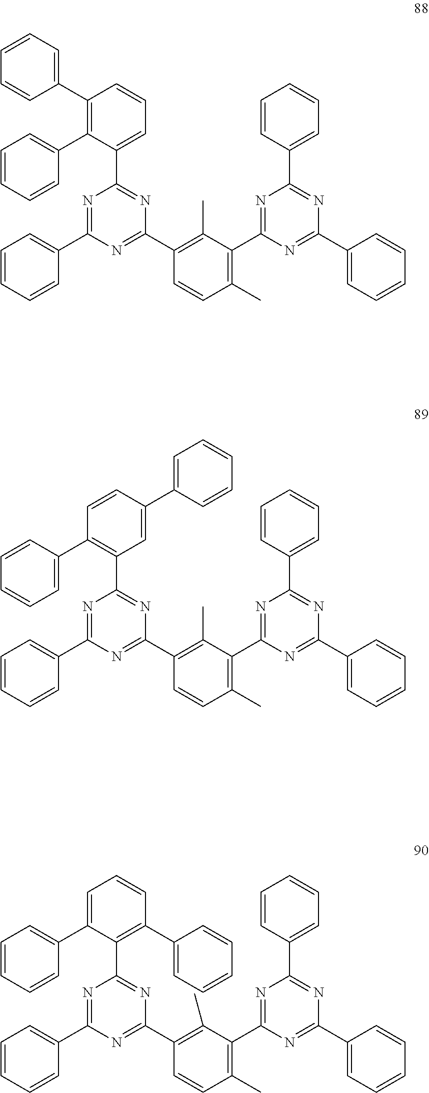 [SMILES: Cc1ccc(-c2nc(-c3ccccc3)nc(-c3cccc(-c4ccccc4)c3-c3ccccc3)n2)c(C)c1-c1nc(-c2ccccc2)nc(-c2ccccc2)n1 ; Cc1ccc(-c2nc(-c3ccccc3)nc(-c3cc(-c4ccccc4)ccc3-c3ccccc3)n2)c(C)c1-c1nc(-c2ccccc2)nc(-c2ccccc2)n1 ; Cc1ccc(-c2nc(-c3ccccc3)nc(-c3c(-c4ccccc4)cccc3-c3ccccc3)n2)c(C)c1-c1nc(-c2ccccc2)nc(-c2ccccc2)n1]

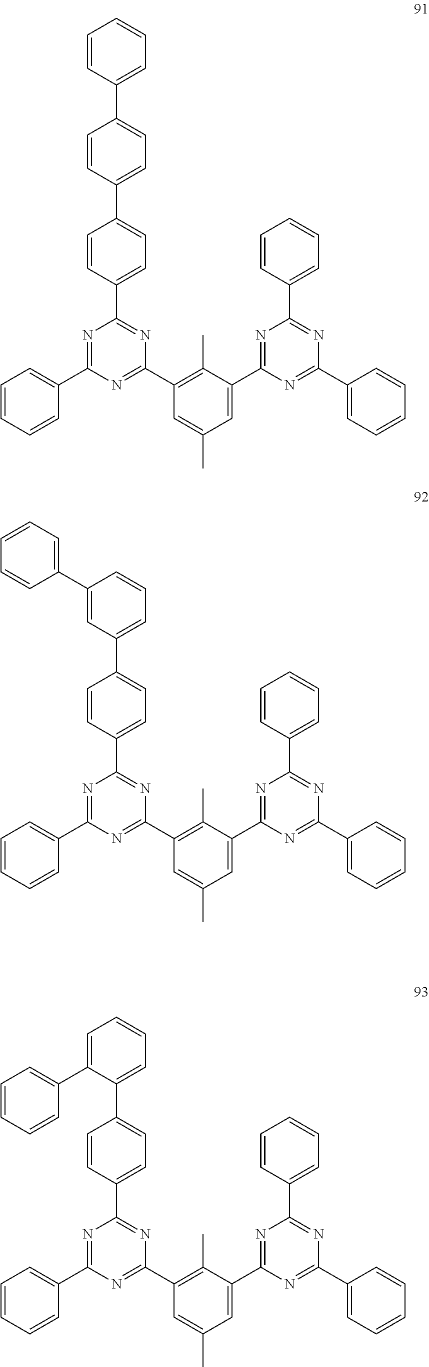 [SMILES: Cc1cc(-c2nc(-c3ccccc3)nc(-c3ccccc3)n2)c(C)c(-c2nc(-c3ccccc3)nc(-c3ccc(-c4ccc(-c5ccccc5)cc4)cc3)n2)c1 ; Cc1cc(-c2nc(-c3ccccc3)nc(-c3ccccc3)n2)c(C)c(-c2nc(-c3ccccc3)nc(-c3ccc(-c4cccc(-c5ccccc5)c4)cc3)n2)c1 ; Cc1cc(-c2nc(-c3ccccc3)nc(-c3ccccc3)n2)c(C)c(-c2nc(-c3ccccc3)nc(-c3ccc(-c4ccccc4-c4ccccc4)cc3)n2)c1]

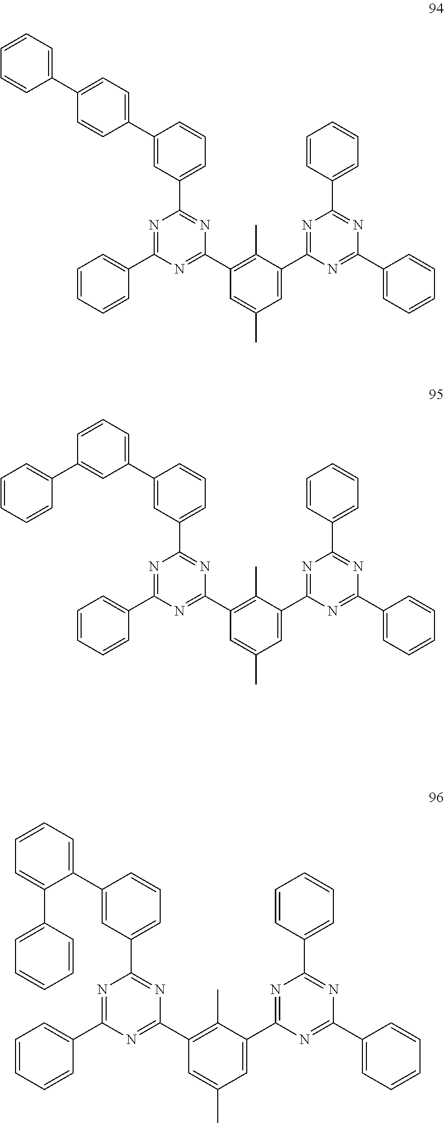 [SMILES: Cc1cc(-c2nc(-c3ccccc3)nc(-c3ccccc3)n2)c(C)c(-c2nc(-c3ccccc3)nc(-c3cccc(-c4ccc(-c5ccccc5)cc4)c3)n2)c1 ; Cc1cc(-c2nc(-c3ccccc3)nc(-c3ccccc3)n2)c(C)c(-c2nc(-c3ccccc3)nc(-c3cccc(-c4cccc(-c5ccccc5)c4)c3)n2)c1 ; Cc1cc(-c2nc(-c3ccccc3)nc(-c3ccccc3)n2)c(C)c(-c2nc(-c3ccccc3)nc(-c3cccc(-c4ccccc4-c4ccccc4)c3)n2)c1]

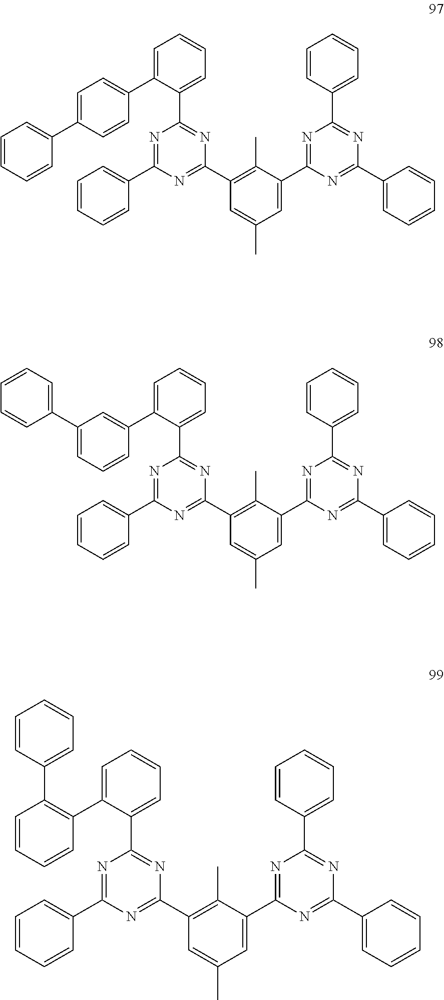 [SMILES: Cc1cc(-c2nc(-c3ccccc3)nc(-c3ccccc3)n2)c(C)c(-c2nc(-c3ccccc3)nc(-c3ccccc3-c3ccc(-c4ccccc4)cc3)n2)c1 ; Cc1cc(-c2nc(-c3ccccc3)nc(-c3ccccc3)n2)c(C)c(-c2nc(-c3ccccc3)nc(-c3ccccc3-c3cccc(-c4ccccc4)c3)n2)c1 ; Cc1cc(-c2nc(-c3ccccc3)nc(-c3ccccc3)n2)c(C)c(-c2nc(-c3ccccc3)nc(-c3ccccc3-c3ccccc3-c3ccccc3)n2)c1]

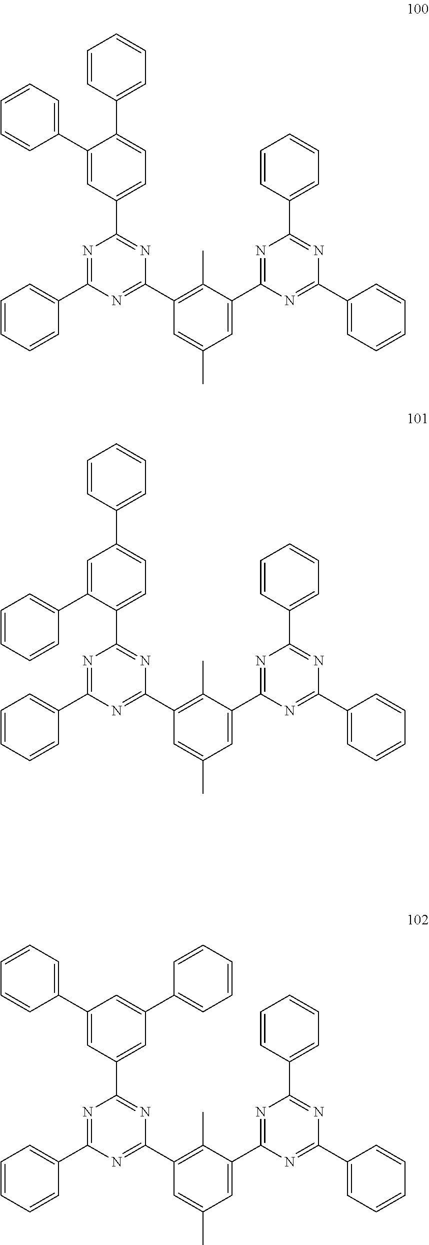 [SMILES: Cc1cc(-c2nc(-c3ccccc3)nc(-c3ccccc3)n2)c(C)c(-c2nc(-c3ccccc3)nc(-c3ccc(-c4ccccc4)c(-c4ccccc4)c3)n2)c1 ; Cc1cc(-c2nc(-c3ccccc3)nc(-c3ccccc3)n2)c(C)c(-c2nc(-c3ccccc3)nc(-c3ccc(-c4ccccc4)cc3-c3ccccc3)n2)c1 ; Cc1cc(-c2nc(-c3ccccc3)nc(-c3ccccc3)n2)c(C)c(-c2nc(-c3ccccc3)nc(-c3cc(-c4ccccc4)cc(-c4ccccc4)c3)n2)c1]

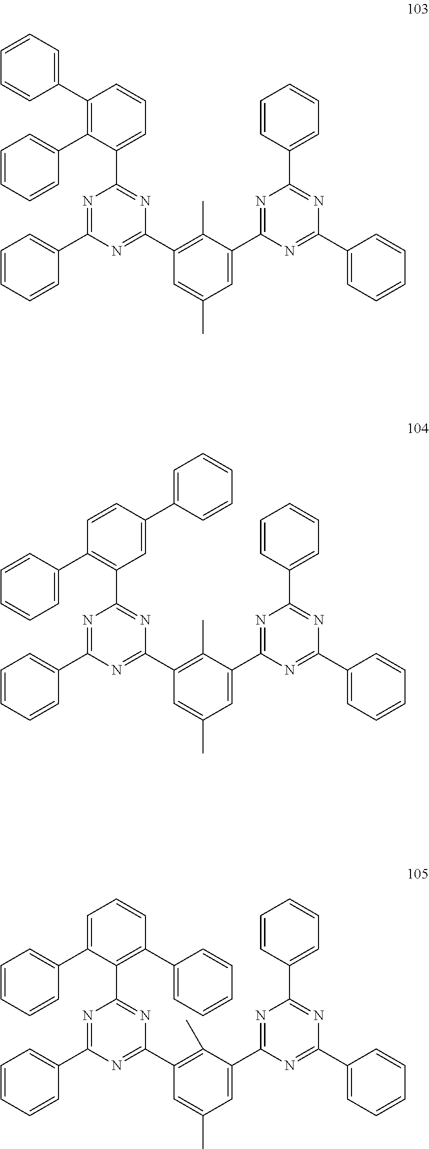 [SMILES: Cc1cc(-c2nc(-c3ccccc3)nc(-c3ccccc3)n2)c(C)c(-c2nc(-c3ccccc3)nc(-c3cccc(-c4ccccc4)c3-c3ccccc3)n2)c1 ; Cc1cc(-c2nc(-c3ccccc3)nc(-c3ccccc3)n2)c(C)c(-c2nc(-c3ccccc3)nc(-c3cc(-c4ccccc4)ccc3-c3ccccc3)n2)c1 ; Cc1cc(-c2nc(-c3ccccc3)nc(-c3ccccc3)n2)c(C)c(-c2nc(-c3ccccc3)nc(-c3c(-c4ccccc4)cccc3-c3ccccc3)n2)c1]

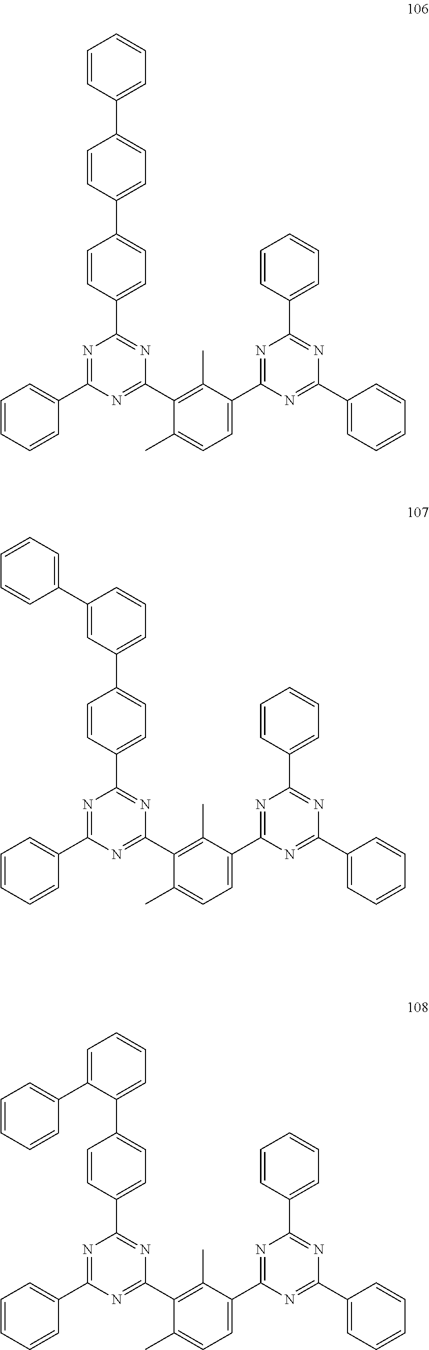 [SMILES: Cc1ccc(-c2nc(-c3ccccc3)nc(-c3ccccc3)n2)c(C)c1-c1nc(-c2ccccc2)nc(-c2ccc(-c3ccc(-c4ccccc4)cc3)cc2)n1 ; Cc1ccc(-c2nc(-c3ccccc3)nc(-c3ccccc3)n2)c(C)c1-c1nc(-c2ccccc2)nc(-c2ccc(-c3cccc(-c4ccccc4)c3)cc2)n1 ; Cc1ccc(-c2nc(-c3ccccc3)nc(-c3ccccc3)n2)c(C)c1-c1nc(-c2ccccc2)nc(-c2ccc(-c3ccccc3-c3ccccc3)cc2)n1]

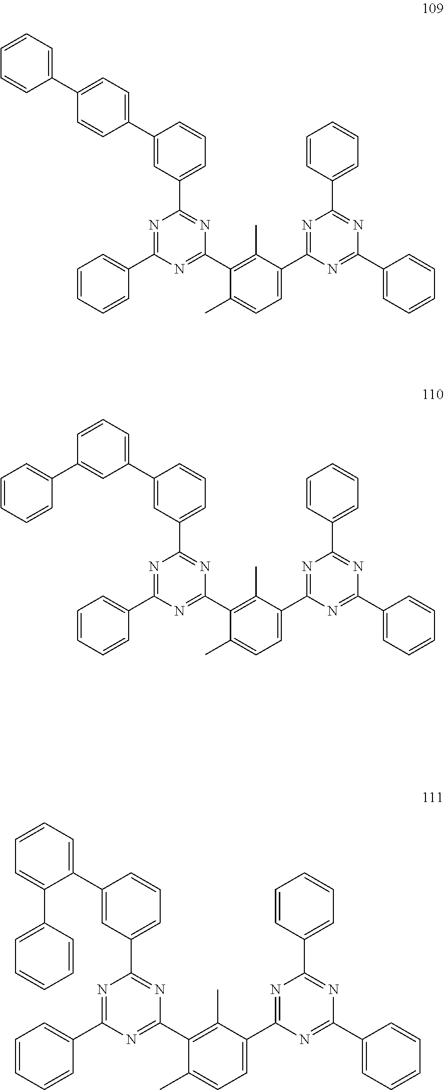 [SMILES: Cc1ccc(-c2nc(-c3ccccc3)nc(-c3ccccc3)n2)c(C)c1-c1nc(-c2ccccc2)nc(-c2cccc(-c3ccc(-c4ccccc4)cc3)c2)n1 ; Cc1ccc(-c2nc(-c3ccccc3)nc(-c3ccccc3)n2)c(C)c1-c1nc(-c2ccccc2)nc(-c2cccc(-c3cccc(-c4ccccc4)c3)c2)n1 ; Cc1ccc(-c2nc(-c3ccccc3)nc(-c3ccccc3)n2)c(C)c1-c1nc(-c2ccccc2)nc(-c2cccc(-c3ccccc3-c3ccccc3)c2)n1]

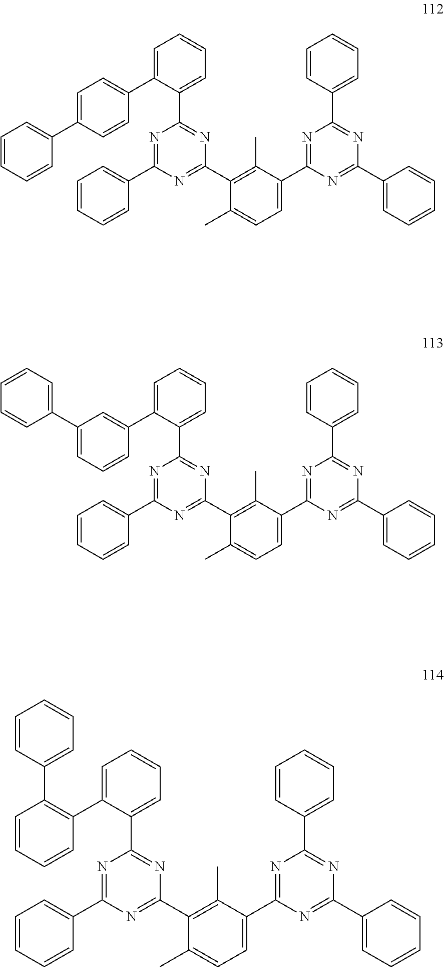 [SMILES: Cc1ccc(-c2nc(-c3ccccc3)nc(-c3ccccc3)n2)c(C)c1-c1nc(-c2ccccc2)nc(-c2ccccc2-c2ccc(-c3ccccc3)cc2)n1 ; Cc1ccc(-c2nc(-c3ccccc3)nc(-c3ccccc3)n2)c(C)c1-c1nc(-c2ccccc2)nc(-c2ccccc2-c2cccc(-c3ccccc3)c2)n1 ; Cc1ccc(-c2nc(-c3ccccc3)nc(-c3ccccc3)n2)c(C)c1-c1nc(-c2ccccc2)nc(-c2ccccc2-c2ccccc2-c2ccccc2)n1]

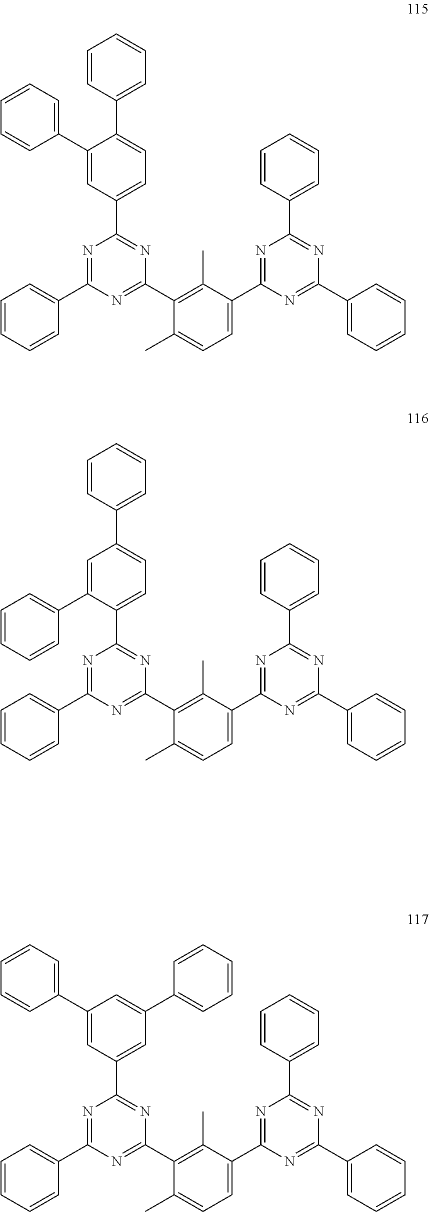 [SMILES: Cc1ccc(-c2nc(-c3ccccc3)nc(-c3ccccc3)n2)c(C)c1-c1nc(-c2ccccc2)nc(-c2ccc(-c3ccccc3)c(-c3ccccc3)c2)n1 ; Cc1ccc(-c2nc(-c3ccccc3)nc(-c3ccccc3)n2)c(C)c1-c1nc(-c2ccccc2)nc(-c2ccc(-c3ccccc3)cc2-c2ccccc2)n1 ; Cc1ccc(-c2nc(-c3ccccc3)nc(-c3ccccc3)n2)c(C)c1-c1nc(-c2ccccc2)nc(-c2cc(-c3ccccc3)cc(-c3ccccc3)c2)n1]

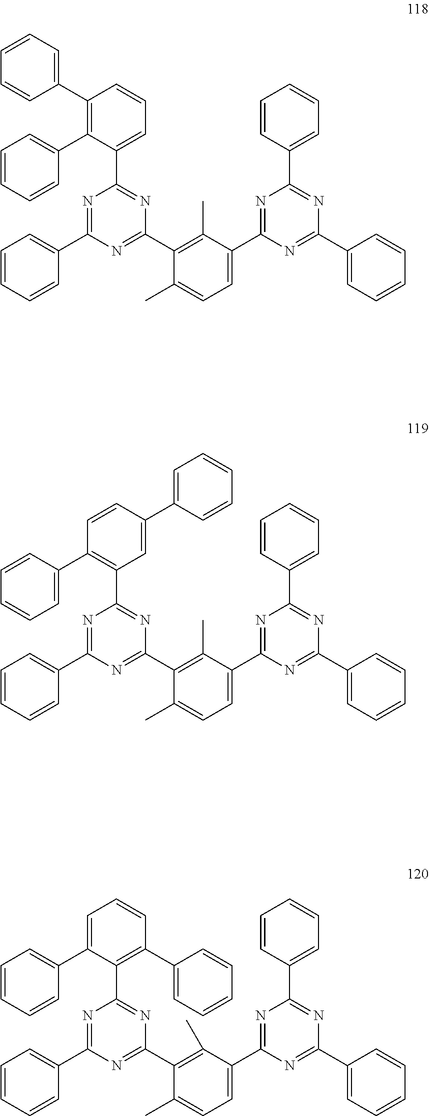 [SMILES: Cc1ccc(-c2nc(-c3ccccc3)nc(-c3ccccc3)n2)c(C)c1-c1nc(-c2ccccc2)nc(-c2cccc(-c3ccccc3)c2-c2ccccc2)n1 ; Cc1ccc(-c2nc(-c3ccccc3)nc(-c3ccccc3)n2)c(C)c1-c1nc(-c2ccccc2)nc(-c2cc(-c3ccccc3)ccc2-c2ccccc2)n1 ; Cc1ccc(-c2nc(-c3ccccc3)nc(-c3ccccc3)n2)c(C)c1-c1nc(-c2ccccc2)nc(-c2c(-c3ccccc3)cccc2-c2ccccc2)n1]

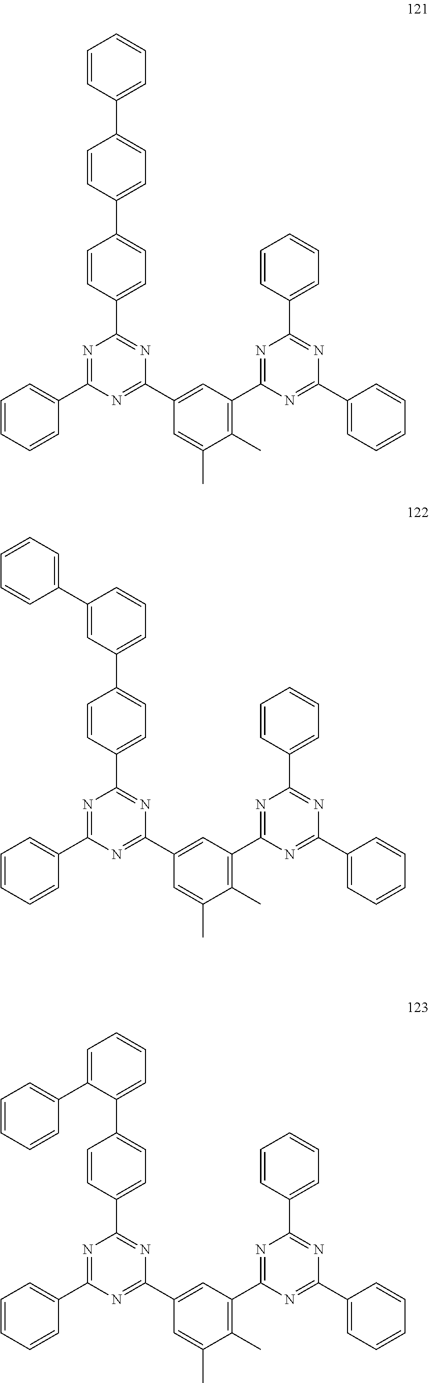 [SMILES: Cc1cc(-c2nc(-c3ccccc3)nc(-c3ccc(-c4ccc(-c5ccccc5)cc4)cc3)n2)cc(-c2nc(-c3ccccc3)nc(-c3ccccc3)n2)c1C ; Cc1cc(-c2nc(-c3ccccc3)nc(-c3ccc(-c4cccc(-c5ccccc5)c4)cc3)n2)cc(-c2nc(-c3ccccc3)nc(-c3ccccc3)n2)c1C ; Cc1cc(-c2nc(-c3ccccc3)nc(-c3ccc(-c4ccccc4-c4ccccc4)cc3)n2)cc(-c2nc(-c3ccccc3)nc(-c3ccccc3)n2)c1C]

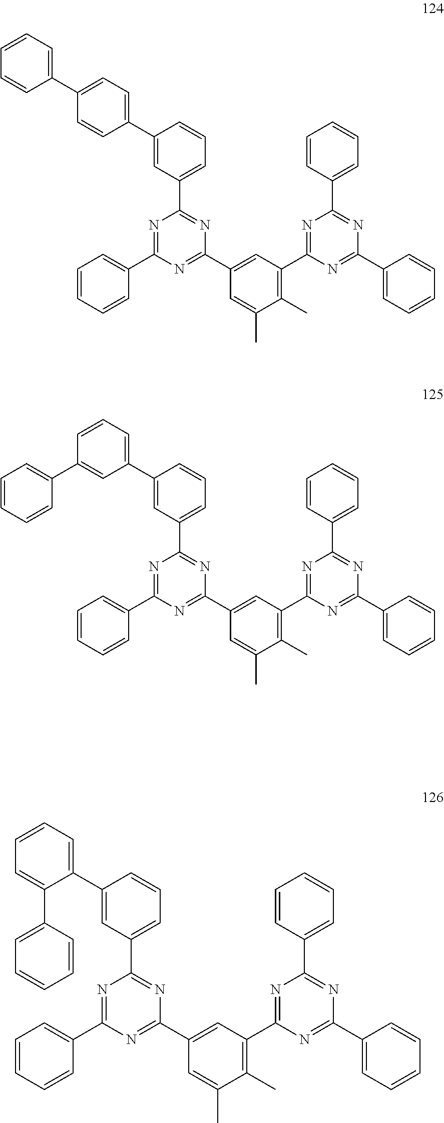 [SMILES: Cc1cc(-c2nc(-c3ccccc3)nc(-c3cccc(-c4ccc(-c5ccccc5)cc4)c3)n2)cc(-c2nc(-c3ccccc3)nc(-c3ccccc3)n2)c1C ; Cc1cc(-c2nc(-c3ccccc3)nc(-c3cccc(-c4cccc(-c5ccccc5)c4)c3)n2)cc(-c2nc(-c3ccccc3)nc(-c3ccccc3)n2)c1C ; Cc1cc(-c2nc(-c3ccccc3)nc(-c3cccc(-c4ccccc4-c4ccccc4)c3)n2)cc(-c2nc(-c3ccccc3)nc(-c3ccccc3)n2)c1C]

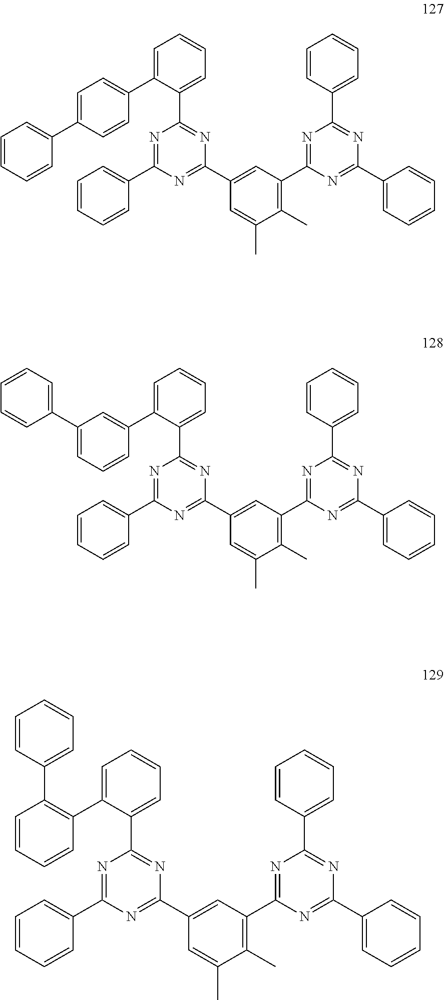 [SMILES: Cc1cc(-c2nc(-c3ccccc3)nc(-c3ccccc3-c3ccc(-c4ccccc4)cc3)n2)cc(-c2nc(-c3ccccc3)nc(-c3ccccc3)n2)c1C ; Cc1cc(-c2nc(-c3ccccc3)nc(-c3ccccc3-c3cccc(-c4ccccc4)c3)n2)cc(-c2nc(-c3ccccc3)nc(-c3ccccc3)n2)c1C ; Cc1cc(-c2nc(-c3ccccc3)nc(-c3ccccc3-c3ccccc3-c3ccccc3)n2)cc(-c2nc(-c3ccccc3)nc(-c3ccccc3)n2)c1C]

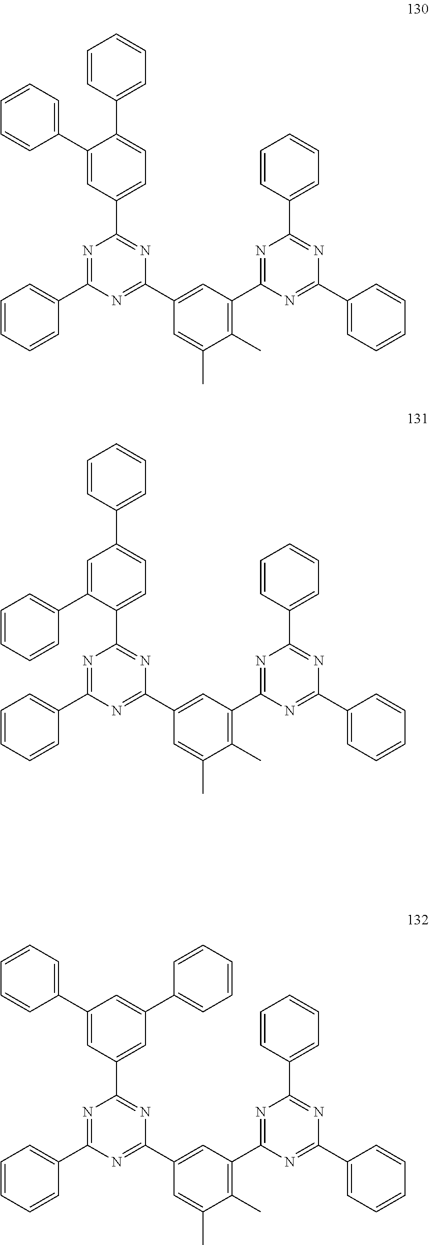 [SMILES: Cc1cc(-c2nc(-c3ccccc3)nc(-c3ccc(-c4ccccc4)c(-c4ccccc4)c3)n2)cc(-c2nc(-c3ccccc3)nc(-c3ccccc3)n2)c1C ; Cc1cc(-c2nc(-c3ccccc3)nc(-c3ccc(-c4ccccc4)cc3-c3ccccc3)n2)cc(-c2nc(-c3ccccc3)nc(-c3ccccc3)n2)c1C ; Cc1cc(-c2nc(-c3ccccc3)nc(-c3cc(-c4ccccc4)cc(-c4ccccc4)c3)n2)cc(-c2nc(-c3ccccc3)nc(-c3ccccc3)n2)c1C]

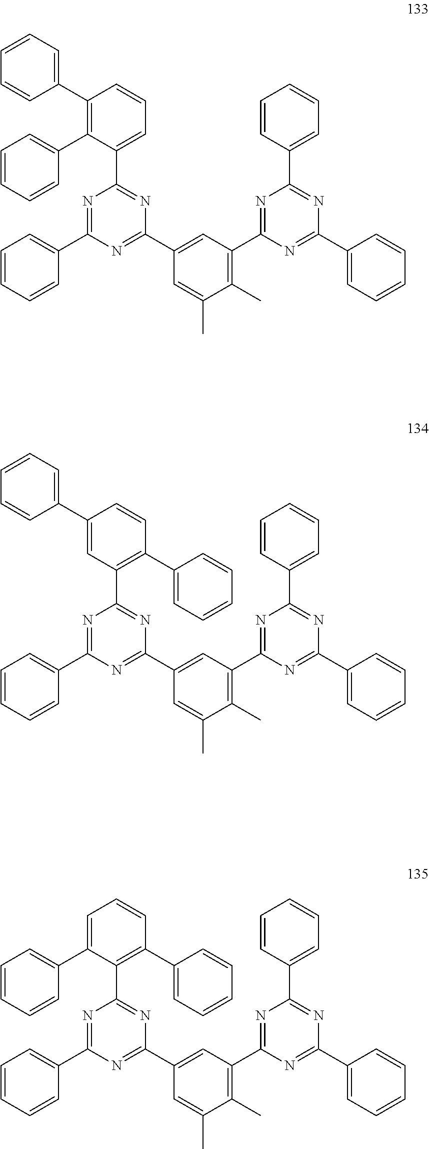 [SMILES: Cc1cc(-c2nc(-c3ccccc3)nc(-c3cccc(-c4ccccc4)c3-c3ccccc3)n2)cc(-c2nc(-c3ccccc3)nc(-c3ccccc3)n2)c1C ; Cc1cc(-c2nc(-c3ccccc3)nc(-c3cc(-c4ccccc4)ccc3-c3ccccc3)n2)cc(-c2nc(-c3ccccc3)nc(-c3ccccc3)n2)c1C ; Cc1cc(-c2nc(-c3ccccc3)nc(-c3c(-c4ccccc4)cccc3-c3ccccc3)n2)cc(-c2nc(-c3ccccc3)nc(-c3ccccc3)n2)c1C]

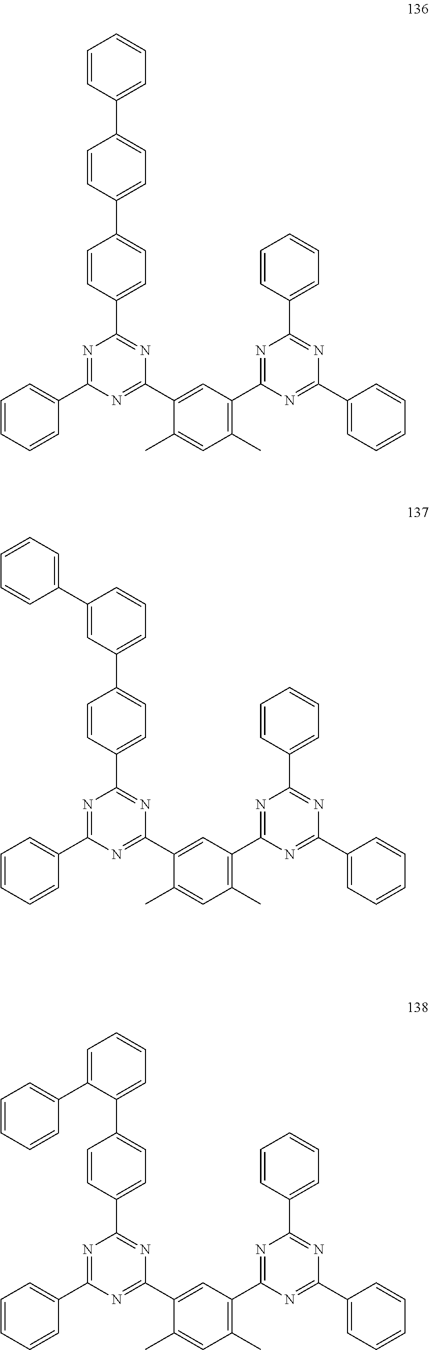 [SMILES: Cc1cc(C)c(-c2nc(-c3ccccc3)nc(-c3ccc(-c4ccc(-c5ccccc5)cc4)cc3)n2)cc1-c1nc(-c2ccccc2)nc(-c2ccccc2)n1 ; Cc1cc(C)c(-c2nc(-c3ccccc3)nc(-c3ccc(-c4cccc(-c5ccccc5)c4)cc3)n2)cc1-c1nc(-c2ccccc2)nc(-c2ccccc2)n1 ; Cc1cc(C)c(-c2nc(-c3ccccc3)nc(-c3ccc(-c4ccccc4-c4ccccc4)cc3)n2)cc1-c1nc(-c2ccccc2)nc(-c2ccccc2)n1]

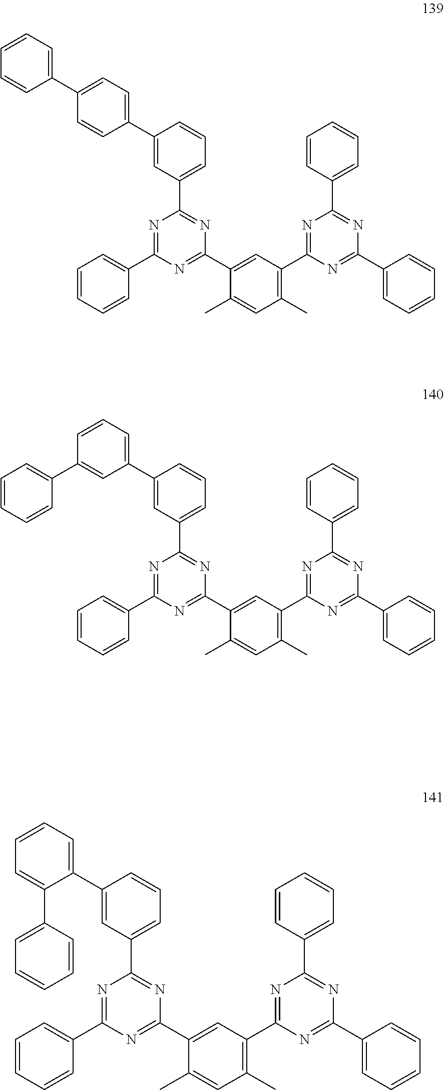 [SMILES: Cc1cc(C)c(-c2nc(-c3ccccc3)nc(-c3cccc(-c4ccc(-c5ccccc5)cc4)c3)n2)cc1-c1nc(-c2ccccc2)nc(-c2ccccc2)n1 ; Cc1cc(C)c(-c2nc(-c3ccccc3)nc(-c3cccc(-c4cccc(-c5ccccc5)c4)c3)n2)cc1-c1nc(-c2ccccc2)nc(-c2ccccc2)n1 ; Cc1cc(C)c(-c2nc(-c3ccccc3)nc(-c3cccc(-c4ccccc4-c4ccccc4)c3)n2)cc1-c1nc(-c2ccccc2)nc(-c2ccccc2)n1]

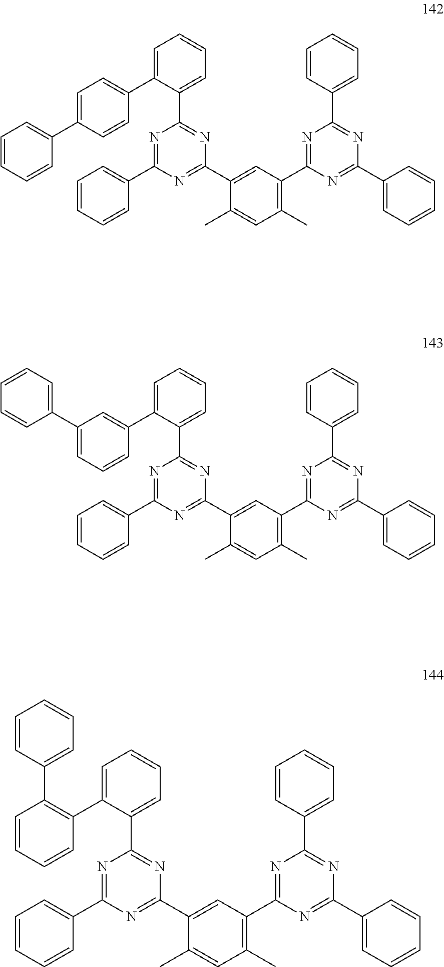 [SMILES: Cc1cc(C)c(-c2nc(-c3ccccc3)nc(-c3ccccc3-c3ccc(-c4ccccc4)cc3)n2)cc1-c1nc(-c2ccccc2)nc(-c2ccccc2)n1 ; Cc1cc(C)c(-c2nc(-c3ccccc3)nc(-c3ccccc3-c3cccc(-c4ccccc4)c3)n2)cc1-c1nc(-c2ccccc2)nc(-c2ccccc2)n1 ; Cc1cc(C)c(-c2nc(-c3ccccc3)nc(-c3ccccc3-c3ccccc3-c3ccccc3)n2)cc1-c1nc(-c2ccccc2)nc(-c2ccccc2)n1]

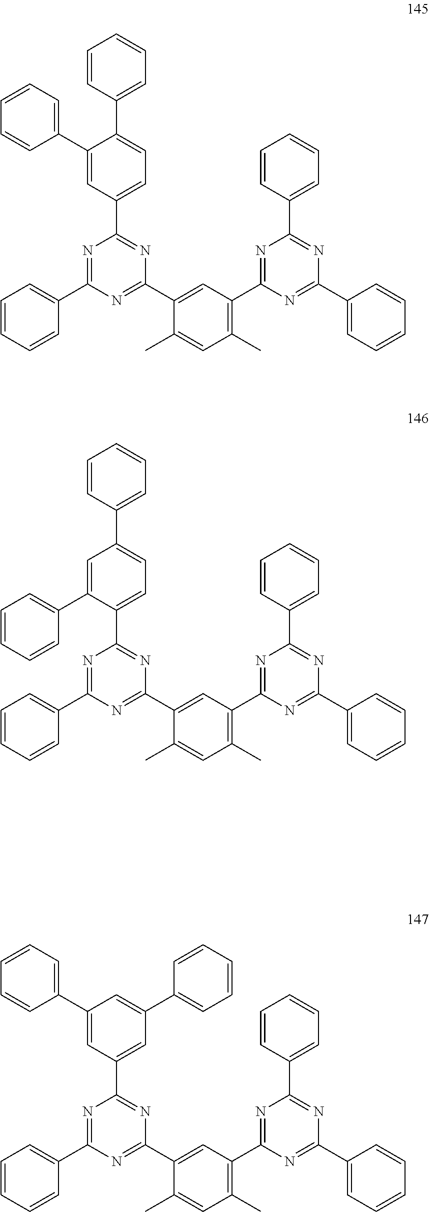 [SMILES: Cc1cc(C)c(-c2nc(-c3ccccc3)nc(-c3ccc(-c4ccccc4)c(-c4ccccc4)c3)n2)cc1-c1nc(-c2ccccc2)nc(-c2ccccc2)n1 ; Cc1cc(C)c(-c2nc(-c3ccccc3)nc(-c3ccc(-c4ccccc4)cc3-c3ccccc3)n2)cc1-c1nc(-c2ccccc2)nc(-c2ccccc2)n1 ; Cc1cc(C)c(-c2nc(-c3ccccc3)nc(-c3cc(-c4ccccc4)cc(-c4ccccc4)c3)n2)cc1-c1nc(-c2ccccc2)nc(-c2ccccc2)n1]

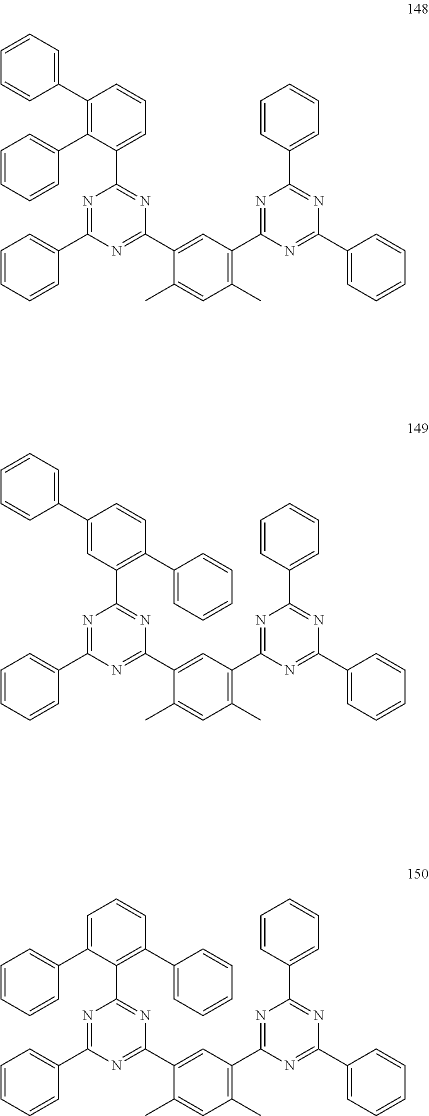 [SMILES: Cc1cc(C)c(-c2nc(-c3ccccc3)nc(-c3cccc(-c4ccccc4)c3-c3ccccc3)n2)cc1-c1nc(-c2ccccc2)nc(-c2ccccc2)n1 ; Cc1cc(C)c(-c2nc(-c3ccccc3)nc(-c3cc(-c4ccccc4)ccc3-c3ccccc3)n2)cc1-c1nc(-c2ccccc2)nc(-c2ccccc2)n1 ; Cc1cc(C)c(-c2nc(-c3ccccc3)nc(-c3c(-c4ccccc4)cccc3-c3ccccc3)n2)cc1-c1nc(-c2ccccc2)nc(-c2ccccc2)n1]

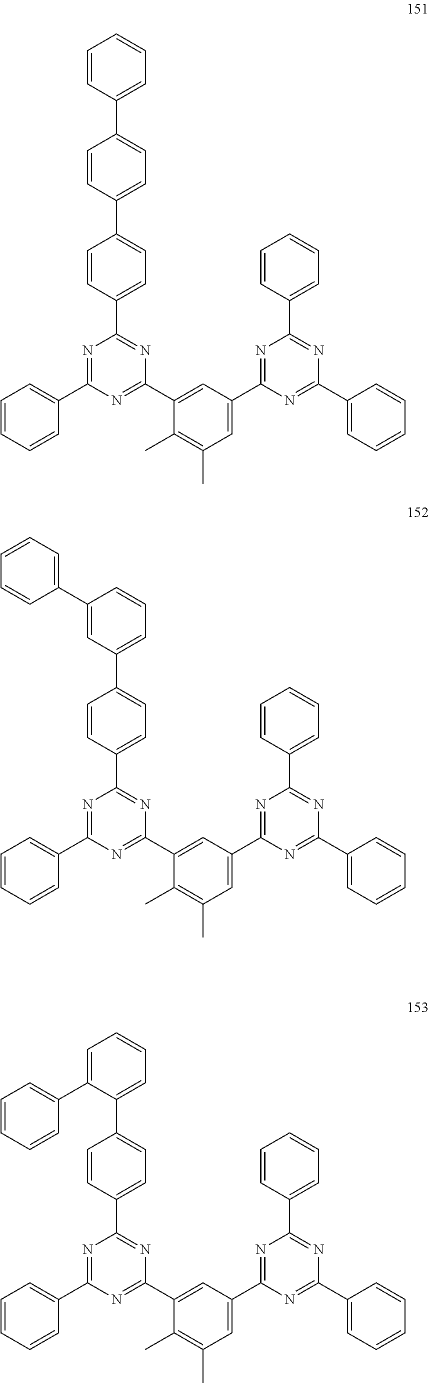 [SMILES: Cc1cc(-c2nc(-c3ccccc3)nc(-c3ccccc3)n2)cc(-c2nc(-c3ccccc3)nc(-c3ccc(-c4ccc(-c5ccccc5)cc4)cc3)n2)c1C ; Cc1cc(-c2nc(-c3ccccc3)nc(-c3ccccc3)n2)cc(-c2nc(-c3ccccc3)nc(-c3ccc(-c4cccc(-c5ccccc5)c4)cc3)n2)c1C ; Cc1cc(-c2nc(-c3ccccc3)nc(-c3ccccc3)n2)cc(-c2nc(-c3ccccc3)nc(-c3ccc(-c4ccccc4-c4ccccc4)cc3)n2)c1C]

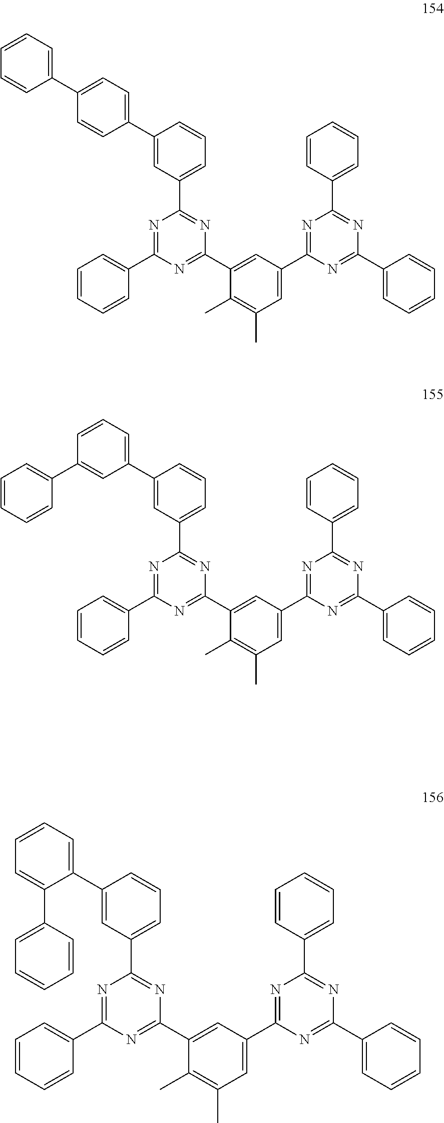 [SMILES: Cc1cc(-c2nc(-c3ccccc3)nc(-c3ccccc3)n2)cc(-c2nc(-c3ccccc3)nc(-c3cccc(-c4ccc(-c5ccccc5)cc4)c3)n2)c1C ; Cc1cc(-c2nc(-c3ccccc3)nc(-c3ccccc3)n2)cc(-c2nc(-c3ccccc3)nc(-c3cccc(-c4cccc(-c5ccccc5)c4)c3)n2)c1C ; Cc1cc(-c2nc(-c3ccccc3)nc(-c3ccccc3)n2)cc(-c2nc(-c3ccccc3)nc(-c3cccc(-c4ccccc4-c4ccccc4)c3)n2)c1C]

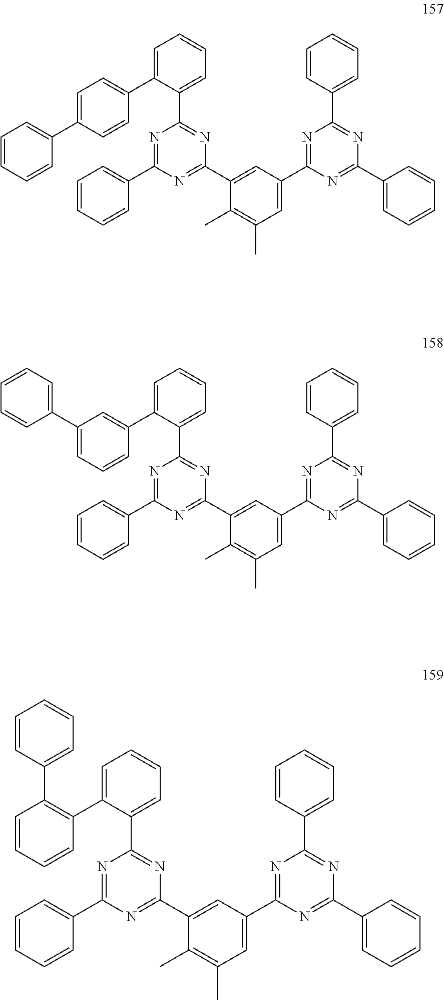 [SMILES: Cc1cc(-c2nc(-c3ccccc3)nc(-c3ccccc3)n2)cc(-c2nc(-c3ccccc3)nc(-c3ccccc3-c3ccc(-c4ccccc4)cc3)n2)c1C ; Cc1cc(-c2nc(-c3ccccc3)nc(-c3ccccc3)n2)cc(-c2nc(-c3ccccc3)nc(-c3ccccc3-c3cccc(-c4ccccc4)c3)n2)c1C ; Cc1cc(-c2nc(-c3ccccc3)nc(-c3ccccc3)n2)cc(-c2nc(-c3ccccc3)nc(-c3ccccc3-c3ccccc3-c3ccccc3)n2)c1C]

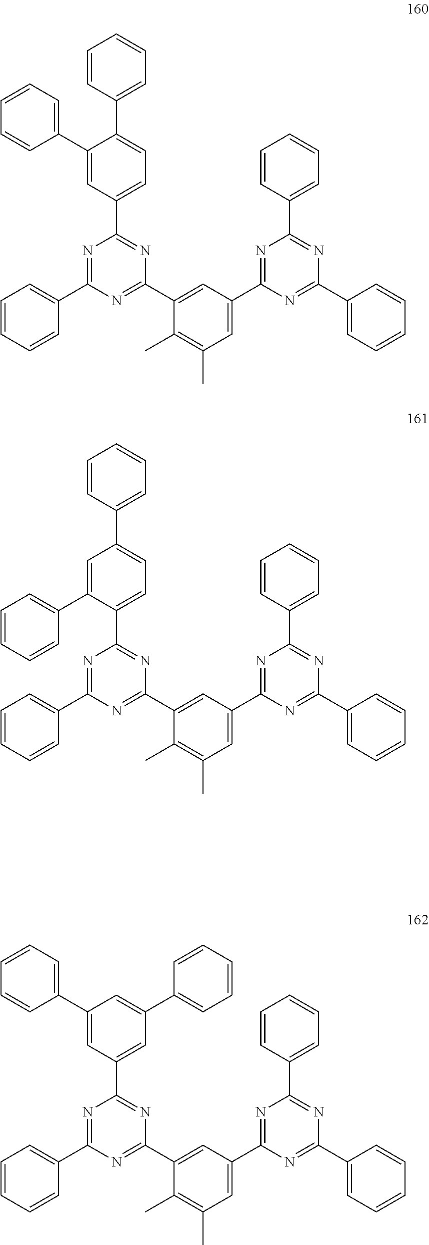 [SMILES: Cc1cc(-c2nc(-c3ccccc3)nc(-c3ccccc3)n2)cc(-c2nc(-c3ccccc3)nc(-c3ccc(-c4ccccc4)c(-c4ccccc4)c3)n2)c1C ; Cc1cc(-c2nc(-c3ccccc3)nc(-c3ccccc3)n2)cc(-c2nc(-c3ccccc3)nc(-c3ccc(-c4ccccc4)cc3-c3ccccc3)n2)c1C ; Cc1cc(-c2nc(-c3ccccc3)nc(-c3ccccc3)n2)cc(-c2nc(-c3ccccc3)nc(-c3cc(-c4ccccc4)cc(-c4ccccc4)c3)n2)c1C]

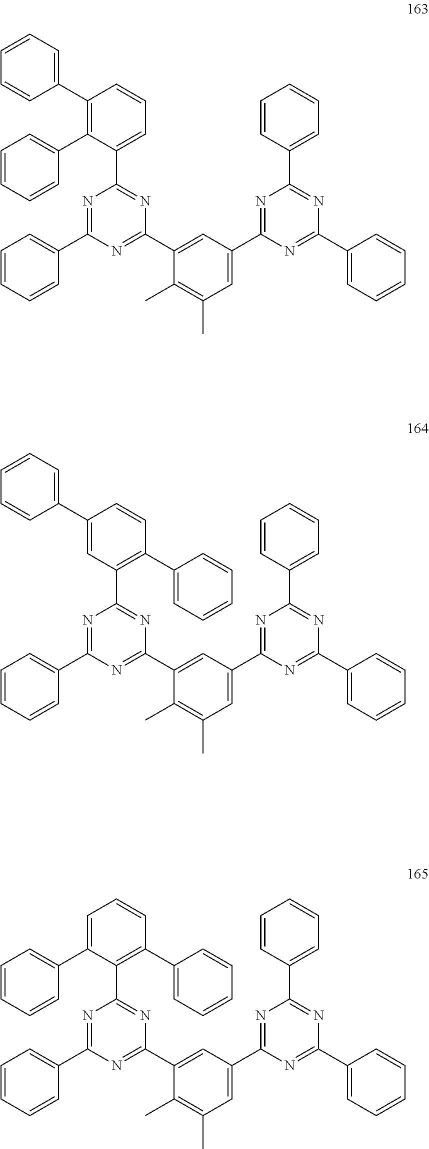 [SMILES: Cc1cc(-c2nc(-c3ccccc3)nc(-c3ccccc3)n2)cc(-c2nc(-c3ccccc3)nc(-c3cccc(-c4ccccc4)c3-c3ccccc3)n2)c1C ; Cc1cc(-c2nc(-c3ccccc3)nc(-c3ccccc3)n2)cc(-c2nc(-c3ccccc3)nc(-c3cc(-c4ccccc4)ccc3-c3ccccc3)n2)c1C ; Cc1cc(-c2nc(-c3ccccc3)nc(-c3ccccc3)n2)cc(-c2nc(-c3ccccc3)nc(-c3c(-c4ccccc4)cccc3-c3ccccc3)n2)c1C]

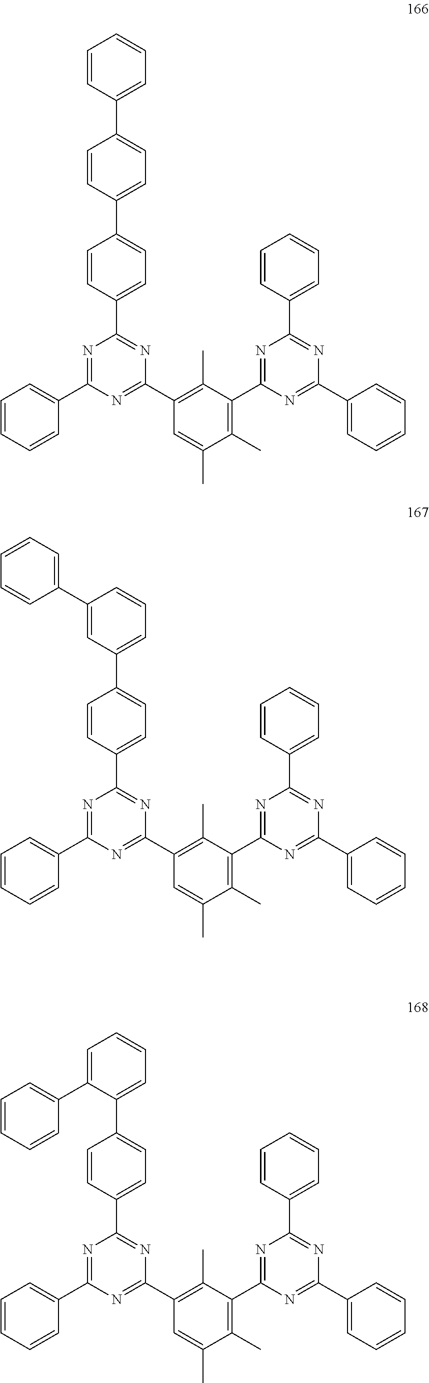 [SMILES: Cc1cc(-c2nc(-c3ccccc3)nc(-c3ccc(-c4ccc(-c5ccccc5)cc4)cc3)n2)c(C)c(-c2nc(-c3ccccc3)nc(-c3ccccc3)n2)c1C ; Cc1cc(-c2nc(-c3ccccc3)nc(-c3ccc(-c4cccc(-c5ccccc5)c4)cc3)n2)c(C)c(-c2nc(-c3ccccc3)nc(-c3ccccc3)n2)c1C ; Cc1cc(-c2nc(-c3ccccc3)nc(-c3ccc(-c4ccccc4-c4ccccc4)cc3)n2)c(C)c(-c2nc(-c3ccccc3)nc(-c3ccccc3)n2)c1C]

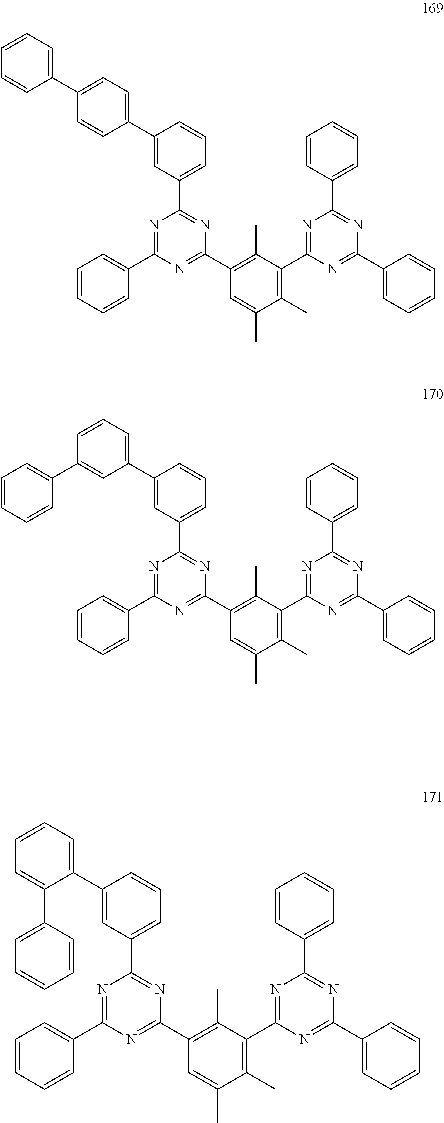 [SMILES: Cc1cc(-c2nc(-c3ccccc3)nc(-c3cccc(-c4ccc(-c5ccccc5)cc4)c3)n2)c(C)c(-c2nc(-c3ccccc3)nc(-c3ccccc3)n2)c1C ; Cc1cc(-c2nc(-c3ccccc3)nc(-c3cccc(-c4cccc(-c5ccccc5)c4)c3)n2)c(C)c(-c2nc(-c3ccccc3)nc(-c3ccccc3)n2)c1C ; Cc1cc(-c2nc(-c3ccccc3)nc(-c3cccc(-c4ccccc4-c4ccccc4)c3)n2)c(C)c(-c2nc(-c3ccccc3)nc(-c3ccccc3)n2)c1C]

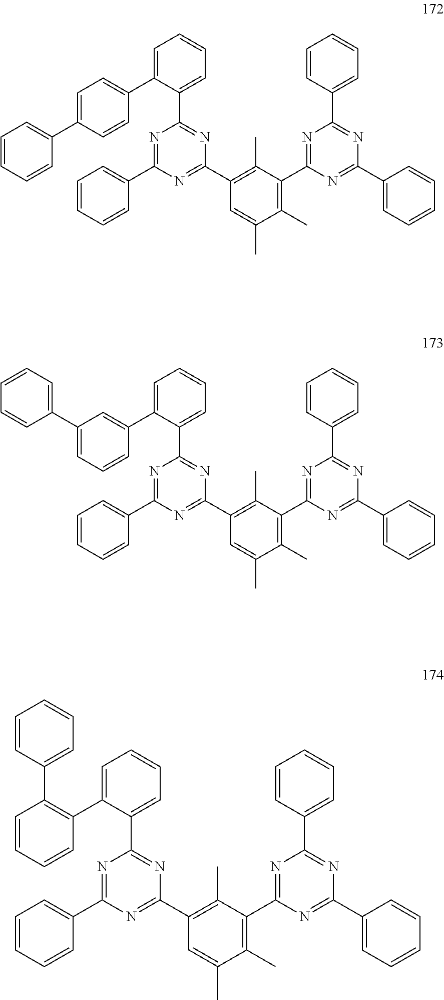 [SMILES: Cc1cc(-c2nc(-c3ccccc3)nc(-c3ccccc3-c3ccc(-c4ccccc4)cc3)n2)c(C)c(-c2nc(-c3ccccc3)nc(-c3ccccc3)n2)c1C ; Cc1cc(-c2nc(-c3ccccc3)nc(-c3ccccc3-c3cccc(-c4ccccc4)c3)n2)c(C)c(-c2nc(-c3ccccc3)nc(-c3ccccc3)n2)c1C ; Cc1cc(-c2nc(-c3ccccc3)nc(-c3ccccc3-c3ccccc3-c3ccccc3)n2)c(C)c(-c2nc(-c3ccccc3)nc(-c3ccccc3)n2)c1C]

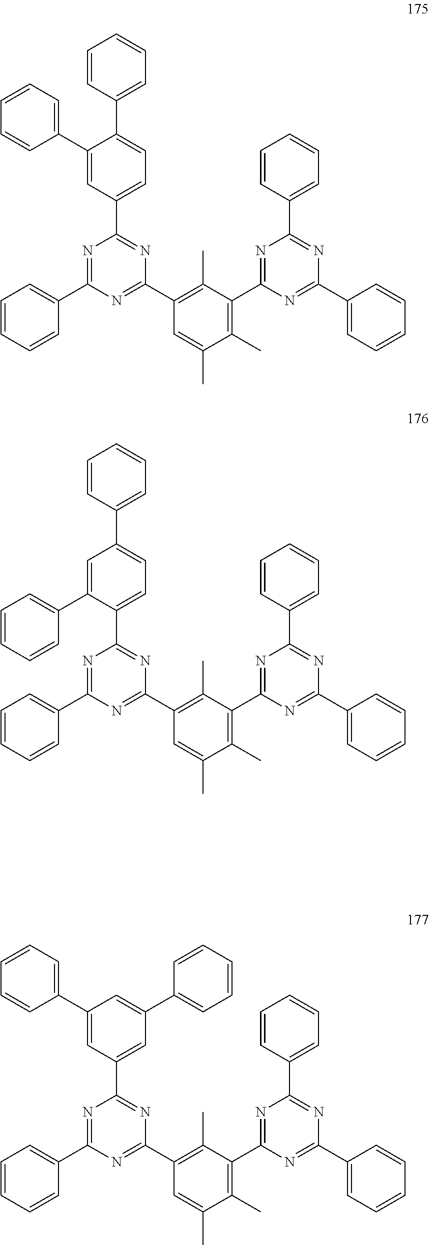 [SMILES: Cc1cc(-c2nc(-c3ccccc3)nc(-c3ccc(-c4ccccc4)c(-c4ccccc4)c3)n2)c(C)c(-c2nc(-c3ccccc3)nc(-c3ccccc3)n2)c1C ; Cc1cc(-c2nc(-c3ccccc3)nc(-c3ccc(-c4ccccc4)cc3-c3ccccc3)n2)c(C)c(-c2nc(-c3ccccc3)nc(-c3ccccc3)n2)c1C ; Cc1cc(-c2nc(-c3ccccc3)nc(-c3cc(-c4ccccc4)cc(-c4ccccc4)c3)n2)c(C)c(-c2nc(-c3ccccc3)nc(-c3ccccc3)n2)c1C]

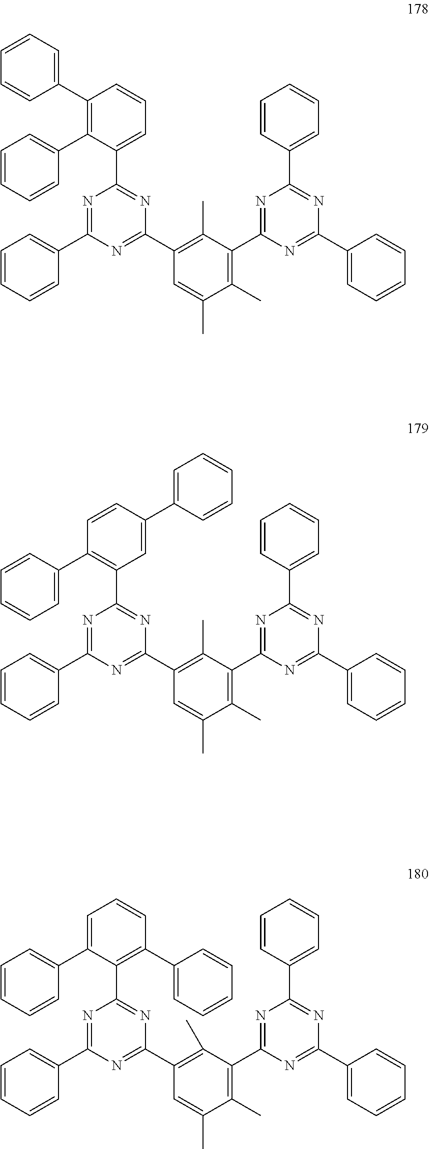 [SMILES: Cc1cc(-c2nc(-c3ccccc3)nc(-c3cccc(-c4ccccc4)c3-c3ccccc3)n2)c(C)c(-c2nc(-c3ccccc3)nc(-c3ccccc3)n2)c1C ; Cc1cc(-c2nc(-c3ccccc3)nc(-c3cc(-c4ccccc4)ccc3-c3ccccc3)n2)c(C)c(-c2nc(-c3ccccc3)nc(-c3ccccc3)n2)c1C ; Cc1cc(-c2nc(-c3ccccc3)nc(-c3c(-c4ccccc4)cccc3-c3ccccc3)n2)c(C)c(-c2nc(-c3ccccc3)nc(-c3ccccc3)n2)c1C]

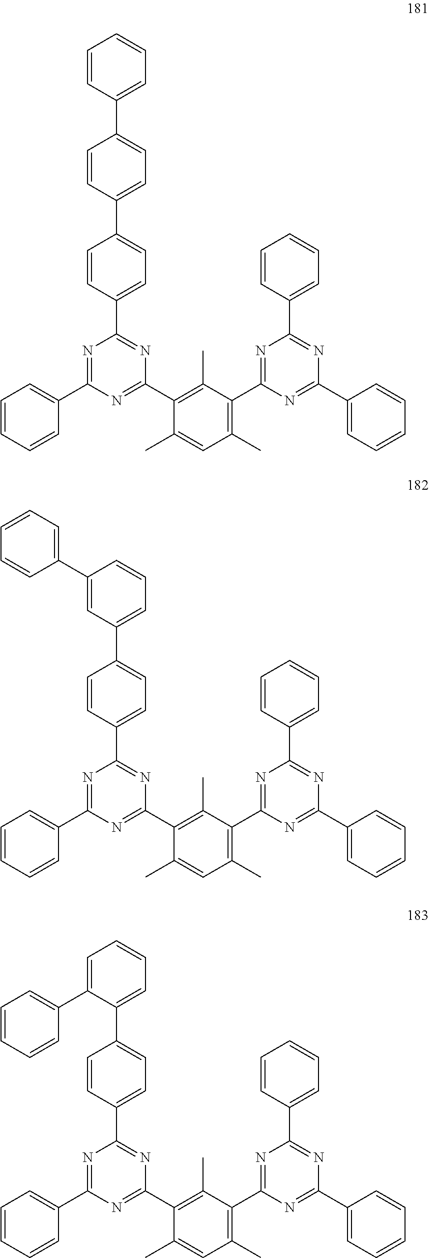 [SMILES: Cc1cc(C)c(-c2nc(-c3ccccc3)nc(-c3ccc(-c4ccc(-c5ccccc5)cc4)cc3)n2)c(C)c1-c1nc(-c2ccccc2)nc(-c2ccccc2)n1 ; Cc1cc(C)c(-c2nc(-c3ccccc3)nc(-c3ccc(-c4cccc(-c5ccccc5)c4)cc3)n2)c(C)c1-c1nc(-c2ccccc2)nc(-c2ccccc2)n1 ; Cc1cc(C)c(-c2nc(-c3ccccc3)nc(-c3ccc(-c4ccccc4-c4ccccc4)cc3)n2)c(C)c1-c1nc(-c2ccccc2)nc(-c2ccccc2)n1]

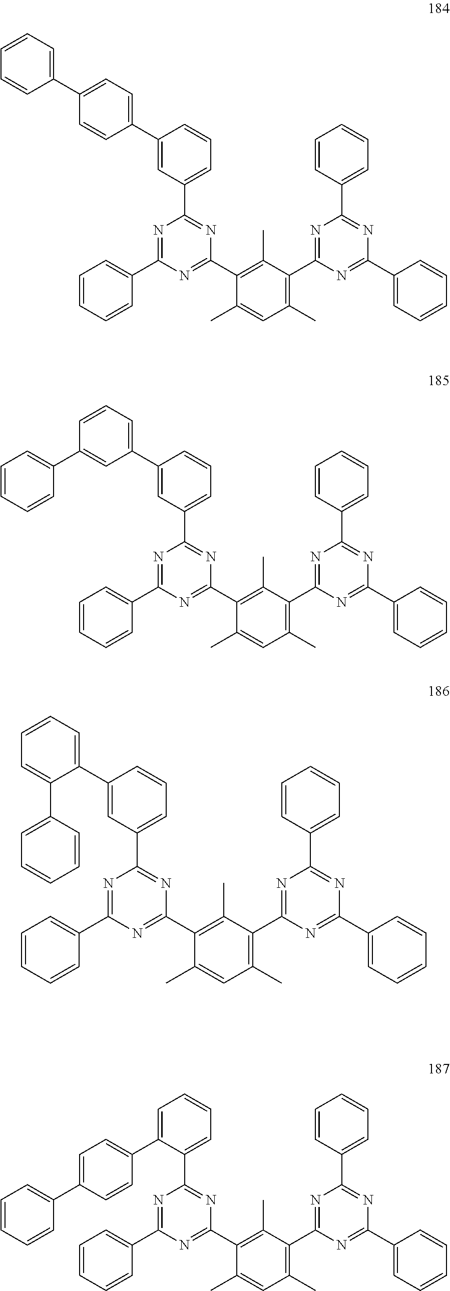 [SMILES: Cc1cc(C)c(-c2nc(-c3ccccc3)nc(-c3cccc(-c4ccc(-c5ccccc5)cc4)c3)n2)c(C)c1-c1nc(-c2ccccc2)nc(-c2ccccc2)n1 ; Cc1cc(C)c(-c2nc(-c3ccccc3)nc(-c3cccc(-c4cccc(-c5ccccc5)c4)c3)n2)c(C)c1-c1nc(-c2ccccc2)nc(-c2ccccc2)n1 ; Cc1cc(C)c(-c2nc(-c3ccccc3)nc(-c3cccc(-c4ccccc4-c4ccccc4)c3)n2)c(C)c1-c1nc(-c2ccccc2)nc(-c2ccccc2)n1 ; Cc1cc(C)c(-c2nc(-c3ccccc3)nc(-c3ccccc3-c3ccc(-c4ccccc4)cc3)n2)c(C)c1-c1nc(-c2ccccc2)nc(-c2ccccc2)n1]

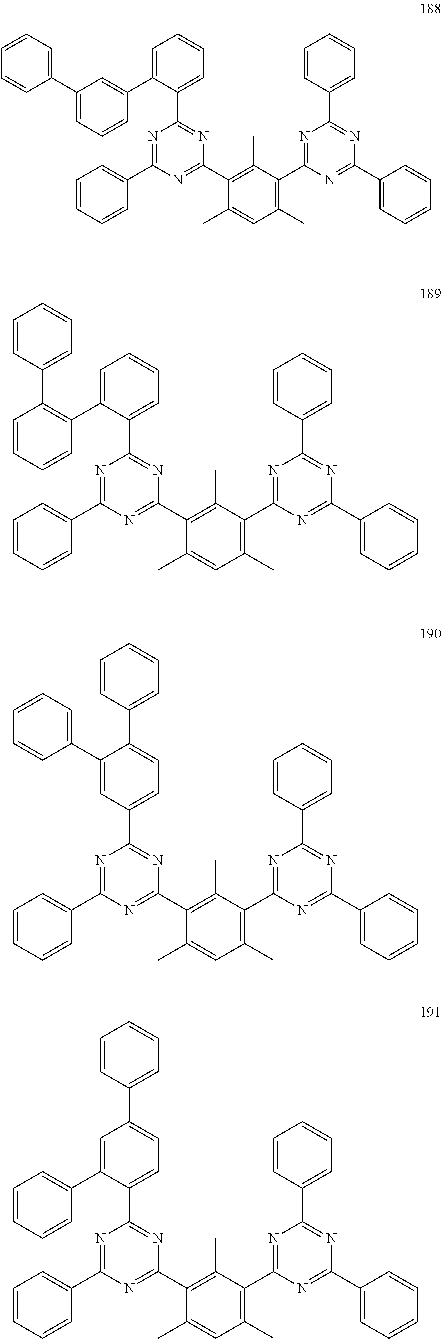 [SMILES: Cc1cc(C)c(-c2nc(-c3ccccc3)nc(-c3ccccc3-c3cccc(-c4ccccc4)c3)n2)c(C)c1-c1nc(-c2ccccc2)nc(-c2ccccc2)n1 ; Cc1cc(C)c(-c2nc(-c3ccccc3)nc(-c3ccccc3-c3ccccc3-c3ccccc3)n2)c(C)c1-c1nc(-c2ccccc2)nc(-c2ccccc2)n1 ; Cc1cc(C)c(-c2nc(-c3ccccc3)nc(-c3ccc(-c4ccccc4)c(-c4ccccc4)c3)n2)c(C)c1-c1nc(-c2ccccc2)nc(-c2ccccc2)n1 ; Cc1cc(C)c(-c2nc(-c3ccccc3)nc(-c3ccc(-c4ccccc4)cc3-c3ccccc3)n2)c(C)c1-c1nc(-c2ccccc2)nc(-c2ccccc2)n1]

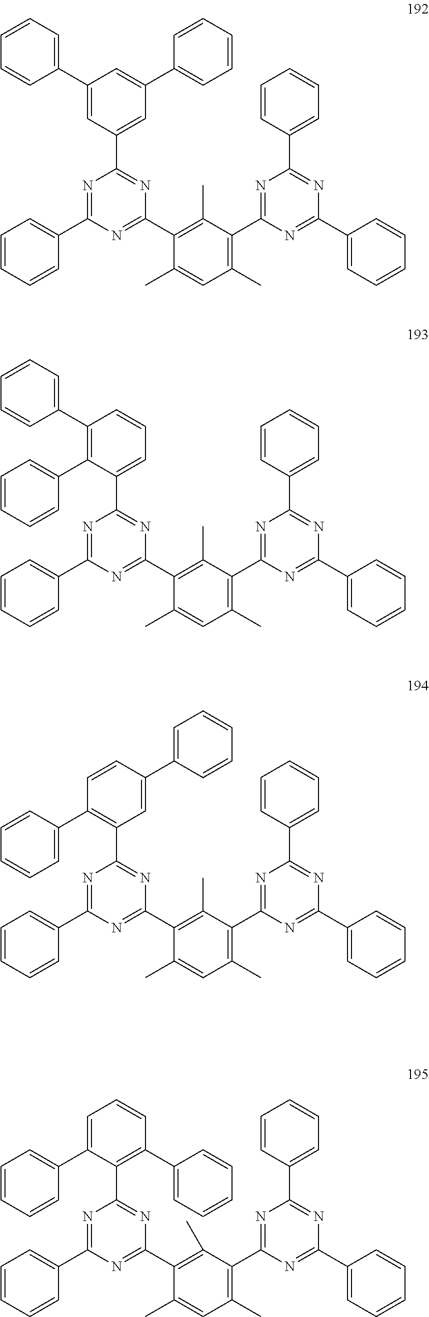 [SMILES: Cc1cc(C)c(-c2nc(-c3ccccc3)nc(-c3cc(-c4ccccc4)cc(-c4ccccc4)c3)n2)c(C)c1-c1nc(-c2ccccc2)nc(-c2ccccc2)n1 ; Cc1cc(C)c(-c2nc(-c3ccccc3)nc(-c3cccc(-c4ccccc4)c3-c3ccccc3)n2)c(C)c1-c1nc(-c2ccccc2)nc(-c2ccccc2)n1 ; Cc1cc(C)c(-c2nc(-c3ccccc3)nc(-c3cc(-c4ccccc4)ccc3-c3ccccc3)n2)c(C)c1-c1nc(-c2ccccc2)nc(-c2ccccc2)n1 ; Cc1cc(C)c(-c2nc(-c3ccccc3)nc(-c3c(-c4ccccc4)cccc3-c3ccccc3)n2)c(C)c1-c1nc(-c2ccccc2)nc(-c2ccccc2)n1]

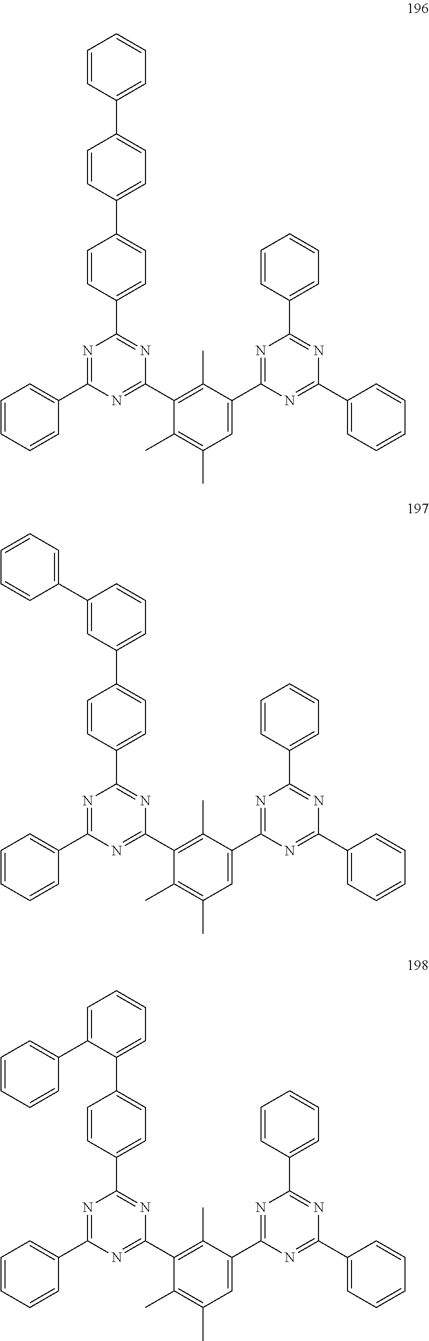 [SMILES: Cc1cc(-c2nc(-c3ccccc3)nc(-c3ccccc3)n2)c(C)c(-c2nc(-c3ccccc3)nc(-c3ccc(-c4ccc(-c5ccccc5)cc4)cc3)n2)c1C ; Cc1cc(-c2nc(-c3ccccc3)nc(-c3ccccc3)n2)c(C)c(-c2nc(-c3ccccc3)nc(-c3ccc(-c4cccc(-c5ccccc5)c4)cc3)n2)c1C ; Cc1cc(-c2nc(-c3ccccc3)nc(-c3ccccc3)n2)c(C)c(-c2nc(-c3ccccc3)nc(-c3ccc(-c4ccccc4-c4ccccc4)cc3)n2)c1C]

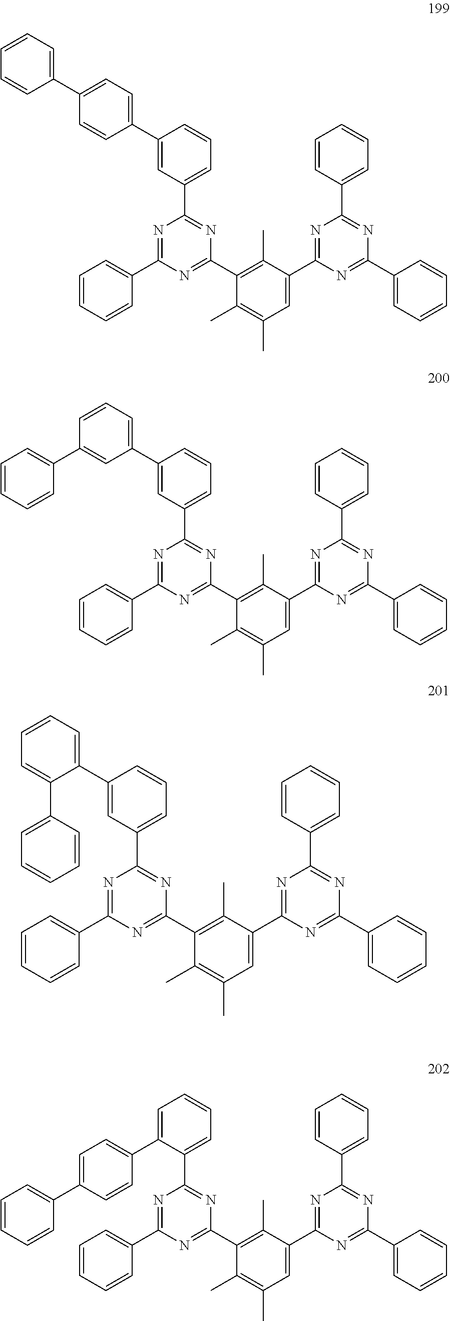 [SMILES: Cc1cc(-c2nc(-c3ccccc3)nc(-c3ccccc3)n2)c(C)c(-c2nc(-c3ccccc3)nc(-c3cccc(-c4ccc(-c5ccccc5)cc4)c3)n2)c1C ; Cc1cc(-c2nc(-c3ccccc3)nc(-c3ccccc3)n2)c(C)c(-c2nc(-c3ccccc3)nc(-c3cccc(-c4cccc(-c5ccccc5)c4)c3)n2)c1C ; Cc1cc(-c2nc(-c3ccccc3)nc(-c3ccccc3)n2)c(C)c(-c2nc(-c3ccccc3)nc(-c3cccc(-c4ccccc4-c4ccccc4)c3)n2)c1C ; Cc1cc(-c2nc(-c3ccccc3)nc(-c3ccccc3)n2)c(C)c(-c2nc(-c3ccccc3)nc(-c3ccccc3-c3ccc(-c4ccccc4)cc3)n2)c1C]

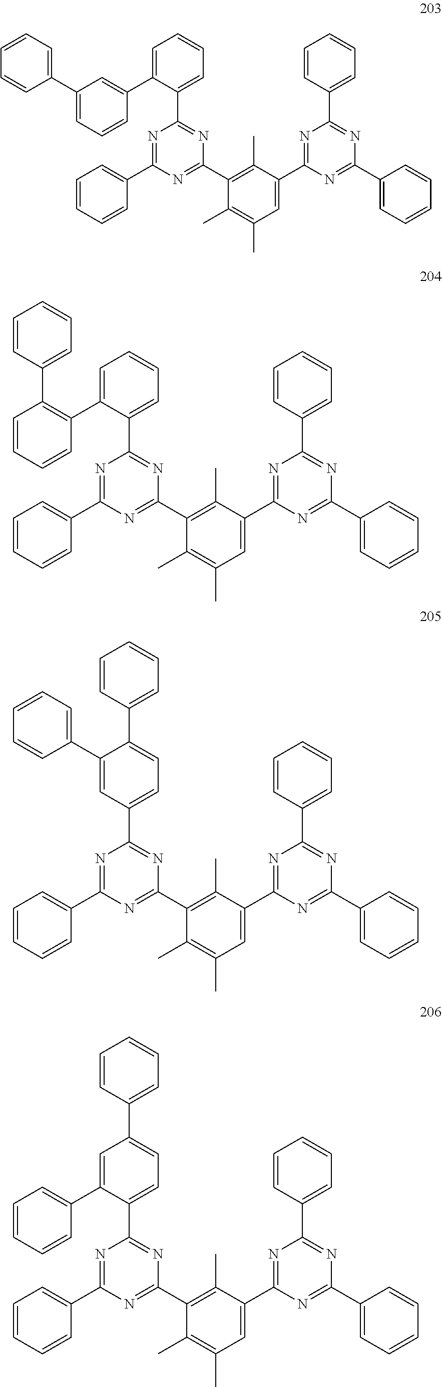 [SMILES: Cc1cc(-c2nc(-c3ccccc3)nc(-c3ccccc3)n2)c(C)c(-c2nc(-c3ccccc3)nc(-c3ccccc3-c3cccc(-c4ccccc4)c3)n2)c1C ; Cc1cc(-c2nc(-c3ccccc3)nc(-c3ccccc3)n2)c(C)c(-c2nc(-c3ccccc3)nc(-c3ccccc3-c3ccccc3-c3ccccc3)n2)c1C ; Cc1cc(-c2nc(-c3ccccc3)nc(-c3ccccc3)n2)c(C)c(-c2nc(-c3ccccc3)nc(-c3ccc(-c4ccccc4)c(-c4ccccc4)c3)n2)c1C ; Cc1cc(-c2nc(-c3ccccc3)nc(-c3ccccc3)n2)c(C)c(-c2nc(-c3ccccc3)nc(-c3ccc(-c4ccccc4)cc3-c3ccccc3)n2)c1C]

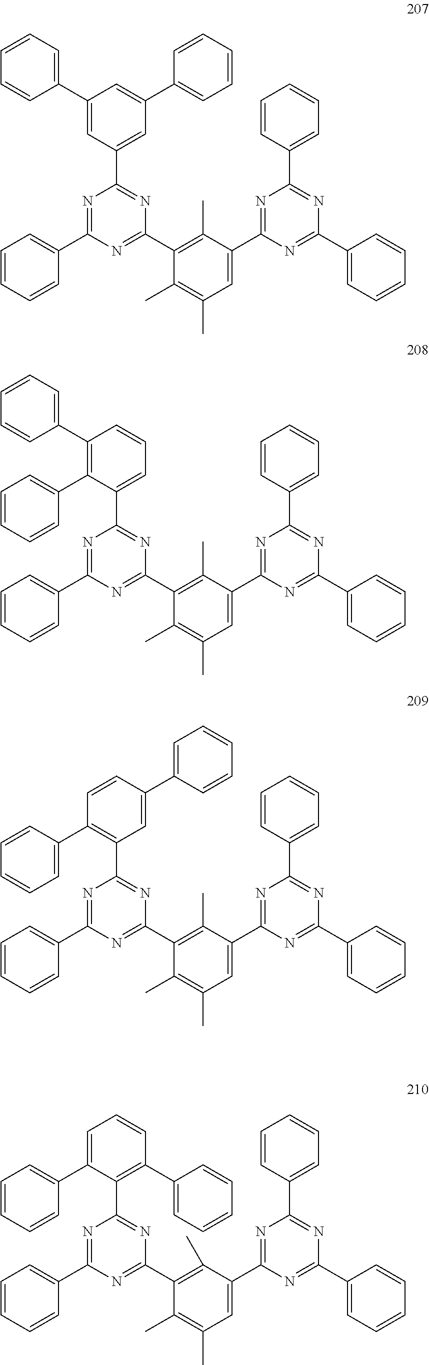 [SMILES: Cc1cc(-c2nc(-c3ccccc3)nc(-c3ccccc3)n2)c(C)c(-c2nc(-c3ccccc3)nc(-c3cc(-c4ccccc4)cc(-c4ccccc4)c3)n2)c1C ; Cc1cc(-c2nc(-c3ccccc3)nc(-c3ccccc3)n2)c(C)c(-c2nc(-c3ccccc3)nc(-c3cccc(-c4ccccc4)c3-c3ccccc3)n2)c1C ; Cc1cc(-c2nc(-c3ccccc3)nc(-c3ccccc3)n2)c(C)c(-c2nc(-c3ccccc3)nc(-c3cc(-c4ccccc4)ccc3-c3ccccc3)n2)c1C ; Cc1cc(-c2nc(-c3ccccc3)nc(-c3ccccc3)n2)c(C)c(-c2nc(-c3ccccc3)nc(-c3c(-c4ccccc4)cccc3-c3ccccc3)n2)c1C]

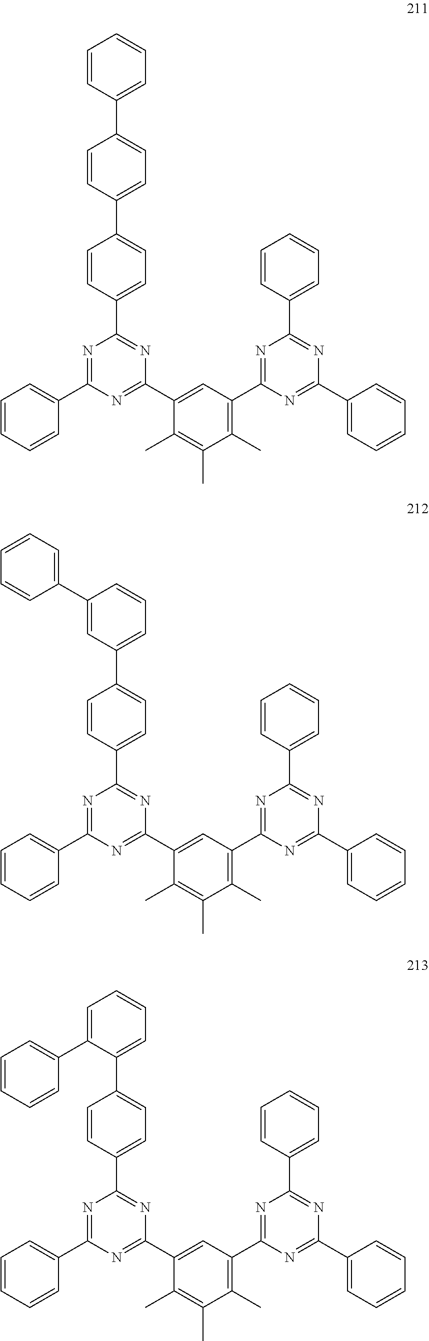 [SMILES: Cc1c(-c2nc(-c3ccccc3)nc(-c3ccccc3)n2)cc(-c2nc(-c3ccccc3)nc(-c3ccc(-c4ccc(-c5ccccc5)cc4)cc3)n2)c(C)c1C ; Cc1c(-c2nc(-c3ccccc3)nc(-c3ccccc3)n2)cc(-c2nc(-c3ccccc3)nc(-c3ccc(-c4cccc(-c5ccccc5)c4)cc3)n2)c(C)c1C ; Cc1c(-c2nc(-c3ccccc3)nc(-c3ccccc3)n2)cc(-c2nc(-c3ccccc3)nc(-c3ccc(-c4ccccc4-c4ccccc4)cc3)n2)c(C)c1C]

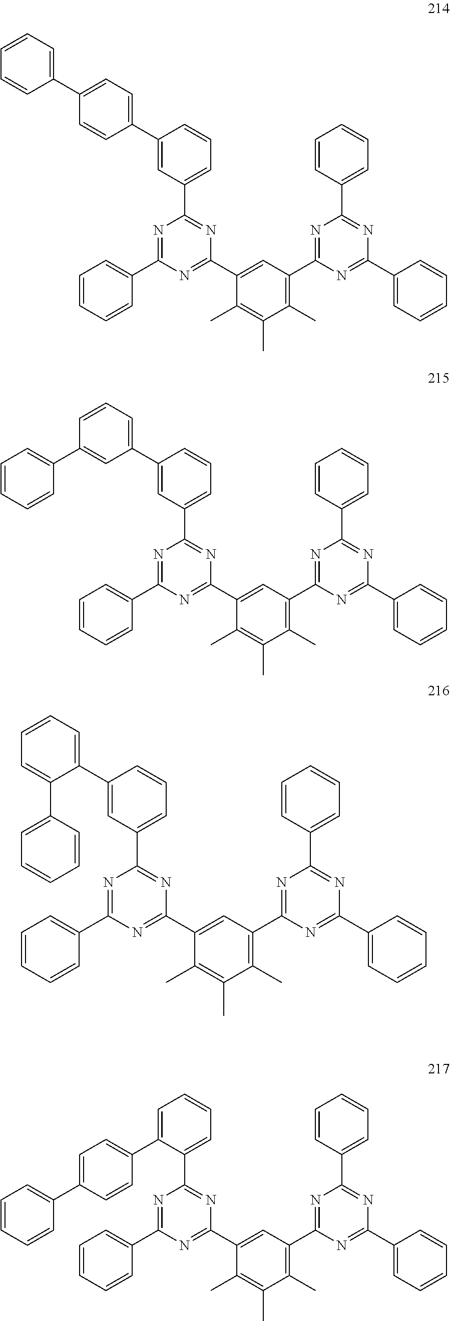 [SMILES: Cc1c(-c2nc(-c3ccccc3)nc(-c3ccccc3)n2)cc(-c2nc(-c3ccccc3)nc(-c3cccc(-c4ccc(-c5ccccc5)cc4)c3)n2)c(C)c1C ; Cc1c(-c2nc(-c3ccccc3)nc(-c3ccccc3)n2)cc(-c2nc(-c3ccccc3)nc(-c3cccc(-c4cccc(-c5ccccc5)c4)c3)n2)c(C)c1C ; Cc1c(-c2nc(-c3ccccc3)nc(-c3ccccc3)n2)cc(-c2nc(-c3ccccc3)nc(-c3cccc(-c4ccccc4-c4ccccc4)c3)n2)c(C)c1C ; Cc1c(-c2nc(-c3ccccc3)nc(-c3ccccc3)n2)cc(-c2nc(-c3ccccc3)nc(-c3ccccc3-c3ccc(-c4ccccc4)cc3)n2)c(C)c1C]

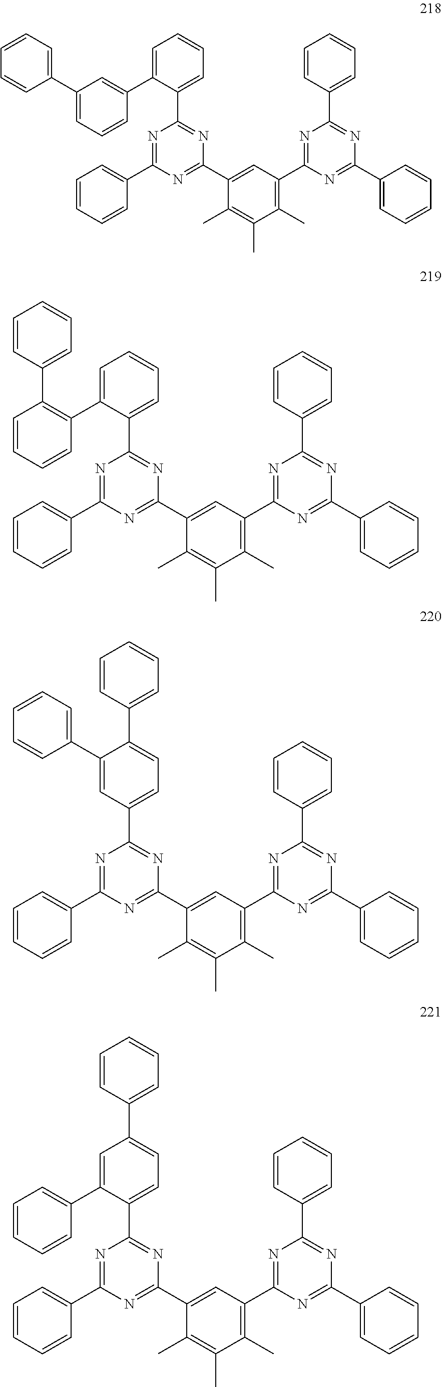 [SMILES: Cc1c(-c2nc(-c3ccccc3)nc(-c3ccccc3)n2)cc(-c2nc(-c3ccccc3)nc(-c3ccccc3-c3cccc(-c4ccccc4)c3)n2)c(C)c1C ; Cc1c(-c2nc(-c3ccccc3)nc(-c3ccccc3)n2)cc(-c2nc(-c3ccccc3)nc(-c3ccccc3-c3ccccc3-c3ccccc3)n2)c(C)c1C ; Cc1c(-c2nc(-c3ccccc3)nc(-c3ccccc3)n2)cc(-c2nc(-c3ccccc3)nc(-c3ccc(-c4ccccc4)c(-c4ccccc4)c3)n2)c(C)c1C ; Cc1c(-c2nc(-c3ccccc3)nc(-c3ccccc3)n2)cc(-c2nc(-c3ccccc3)nc(-c3ccc(-c4ccccc4)cc3-c3ccccc3)n2)c(C)c1C]

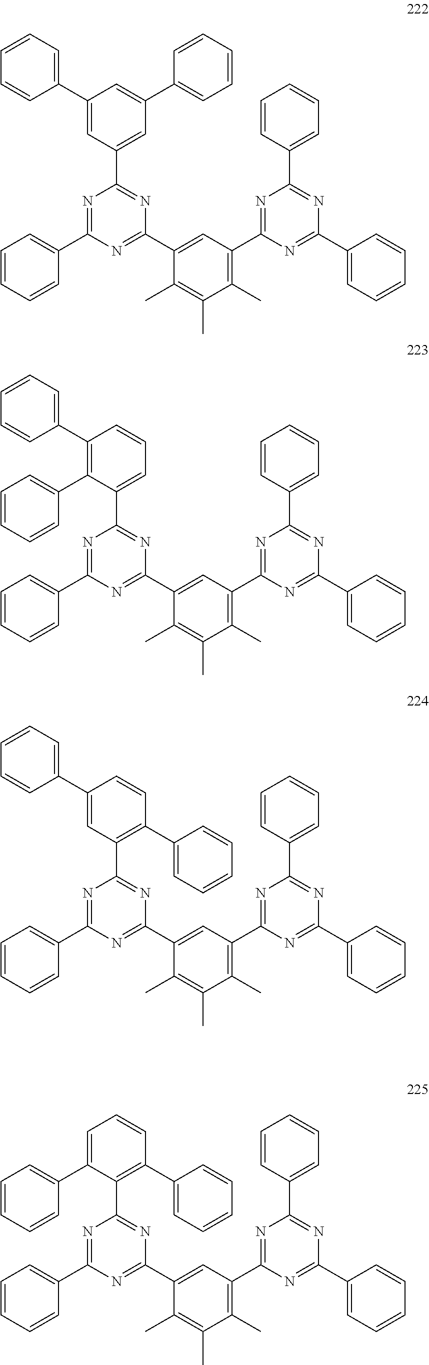 [SMILES: Cc1c(-c2nc(-c3ccccc3)nc(-c3ccccc3)n2)cc(-c2nc(-c3ccccc3)nc(-c3cc(-c4ccccc4)cc(-c4ccccc4)c3)n2)c(C)c1C ; Cc1c(-c2nc(-c3ccccc3)nc(-c3ccccc3)n2)cc(-c2nc(-c3ccccc3)nc(-c3cccc(-c4ccccc4)c3-c3ccccc3)n2)c(C)c1C ; Cc1c(-c2nc(-c3ccccc3)nc(-c3ccccc3)n2)cc(-c2nc(-c3ccccc3)nc(-c3cc(-c4ccccc4)ccc3-c3ccccc3)n2)c(C)c1C ; Cc1c(-c2nc(-c3ccccc3)nc(-c3ccccc3)n2)cc(-c2nc(-c3ccccc3)nc(-c3c(-c4ccccc4)cccc3-c3ccccc3)n2)c(C)c1C]

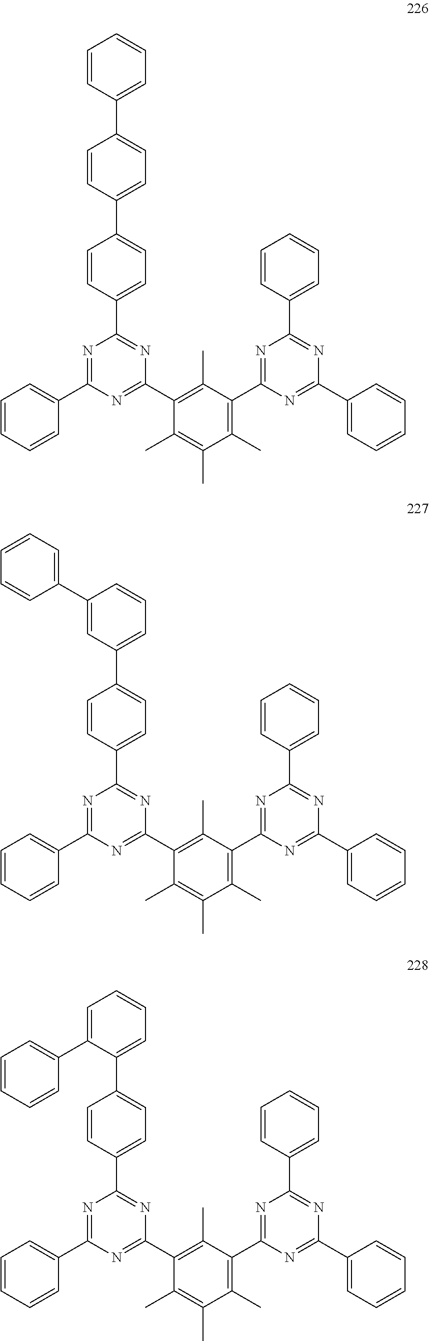 [SMILES: Cc1c(C)c(-c2nc(-c3ccccc3)nc(-c3ccccc3)n2)c(C)c(-c2nc(-c3ccccc3)nc(-c3ccc(-c4ccc(-c5ccccc5)cc4)cc3)n2)c1C ; Cc1c(C)c(-c2nc(-c3ccccc3)nc(-c3ccccc3)n2)c(C)c(-c2nc(-c3ccccc3)nc(-c3ccc(-c4cccc(-c5ccccc5)c4)cc3)n2)c1C ; Cc1c(C)c(-c2nc(-c3ccccc3)nc(-c3ccccc3)n2)c(C)c(-c2nc(-c3ccccc3)nc(-c3ccc(-c4ccccc4-c4ccccc4)cc3)n2)c1C]

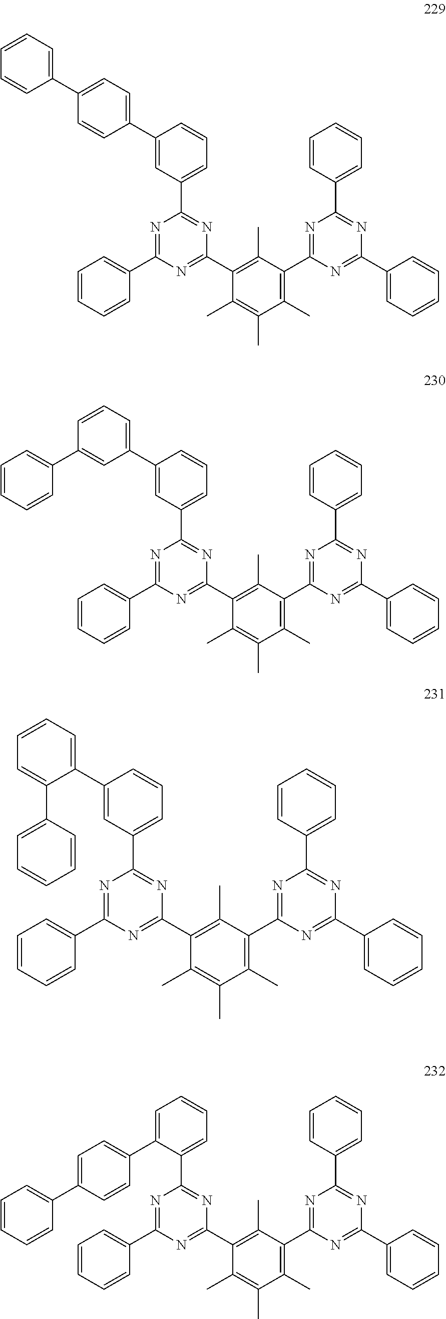 [SMILES: Cc1c(C)c(-c2nc(-c3ccccc3)nc(-c3ccccc3)n2)c(C)c(-c2nc(-c3ccccc3)nc(-c3cccc(-c4ccc(-c5ccccc5)cc4)c3)n2)c1C ; Cc1c(C)c(-c2nc(-c3ccccc3)nc(-c3ccccc3)n2)c(C)c(-c2nc(-c3ccccc3)nc(-c3cccc(-c4cccc(-c5ccccc5)c4)c3)n2)c1C ; Cc1c(C)c(-c2nc(-c3ccccc3)nc(-c3ccccc3)n2)c(C)c(-c2nc(-c3ccccc3)nc(-c3cccc(-c4ccccc4-c4ccccc4)c3)n2)c1C ; Cc1c(C)c(-c2nc(-c3ccccc3)nc(-c3ccccc3)n2)c(C)c(-c2nc(-c3ccccc3)nc(-c3ccccc3-c3ccc(-c4ccccc4)cc3)n2)c1C]

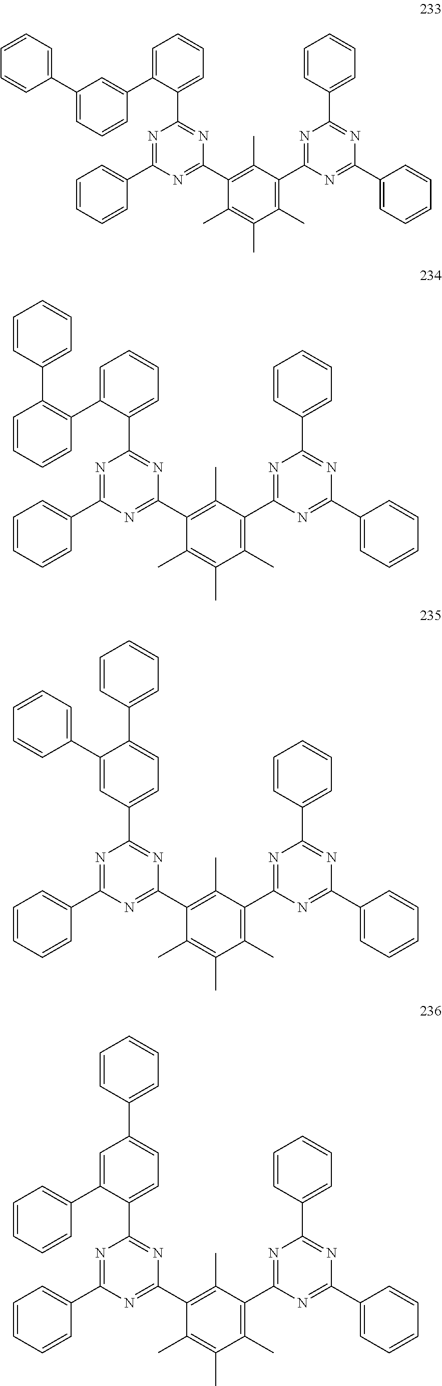 [SMILES: Cc1c(C)c(-c2nc(-c3ccccc3)nc(-c3ccccc3)n2)c(C)c(-c2nc(-c3ccccc3)nc(-c3ccccc3-c3cccc(-c4ccccc4)c3)n2)c1C ; Cc1c(C)c(-c2nc(-c3ccccc3)nc(-c3ccccc3)n2)c(C)c(-c2nc(-c3ccccc3)nc(-c3ccccc3-c3ccccc3-c3ccccc3)n2)c1C ; Cc1c(C)c(-c2nc(-c3ccccc3)nc(-c3ccccc3)n2)c(C)c(-c2nc(-c3ccccc3)nc(-c3ccc(-c4ccccc4)c(-c4ccccc4)c3)n2)c1C ; Cc1c(C)c(-c2nc(-c3ccccc3)nc(-c3ccccc3)n2)c(C)c(-c2nc(-c3ccccc3)nc(-c3ccc(-c4ccccc4)cc3-c3ccccc3)n2)c1C]

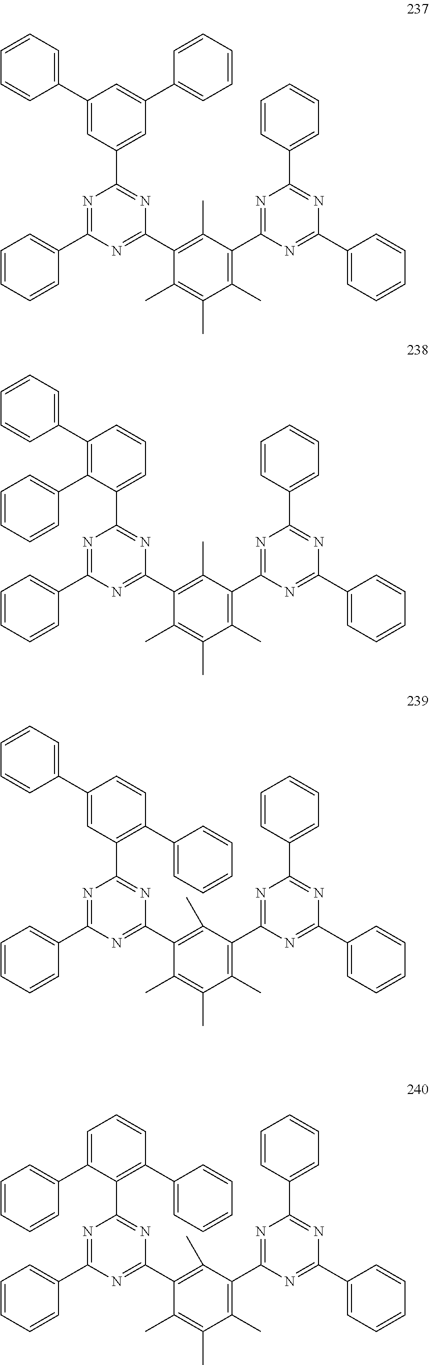 [SMILES: Cc1c(C)c(-c2nc(-c3ccccc3)nc(-c3ccccc3)n2)c(C)c(-c2nc(-c3ccccc3)nc(-c3cc(-c4ccccc4)cc(-c4ccccc4)c3)n2)c1C ; Cc1c(C)c(-c2nc(-c3ccccc3)nc(-c3ccccc3)n2)c(C)c(-c2nc(-c3ccccc3)nc(-c3cccc(-c4ccccc4)c3-c3ccccc3)n2)c1C ; Cc1c(C)c(-c2nc(-c3ccccc3)nc(-c3ccccc3)n2)c(C)c(-c2nc(-c3ccccc3)nc(-c3cc(-c4ccccc4)ccc3-c3ccccc3)n2)c1C ; Cc1c(C)c(-c2nc(-c3ccccc3)nc(-c3ccccc3)n2)c(C)c(-c2nc(-c3ccccc3)nc(-c3c(-c4ccccc4)cccc3-c3ccccc3)n2)c1C]

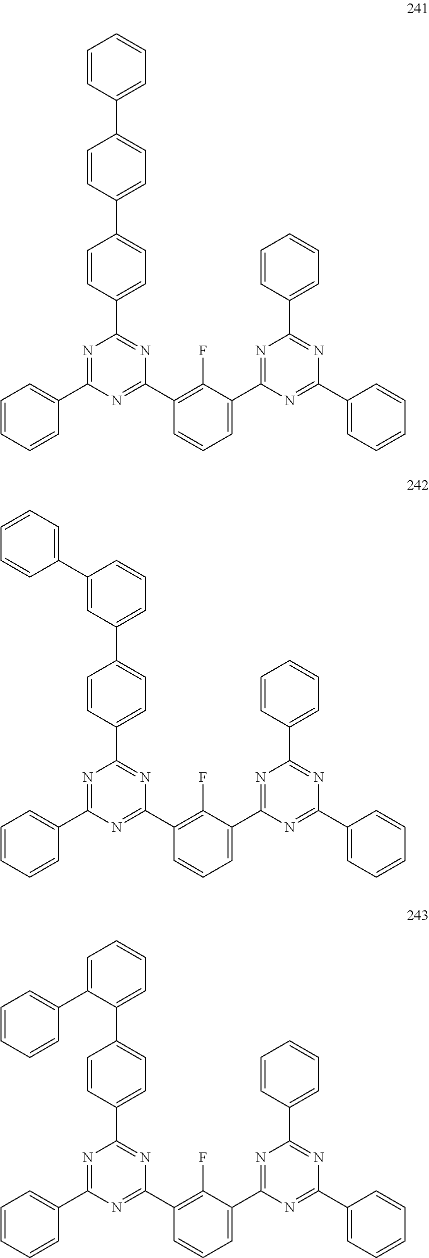 [SMILES: Fc1c(-c2nc(-c3ccccc3)nc(-c3ccccc3)n2)cccc1-c1nc(-c2ccccc2)nc(-c2ccc(-c3ccc(-c4ccccc4)cc3)cc2)n1 ; Fc1c(-c2nc(-c3ccccc3)nc(-c3ccccc3)n2)cccc1-c1nc(-c2ccccc2)nc(-c2ccc(-c3cccc(-c4ccccc4)c3)cc2)n1 ; Fc1c(-c2nc(-c3ccccc3)nc(-c3ccccc3)n2)cccc1-c1nc(-c2ccccc2)nc(-c2ccc(-c3ccccc3-c3ccccc3)cc2)n1]

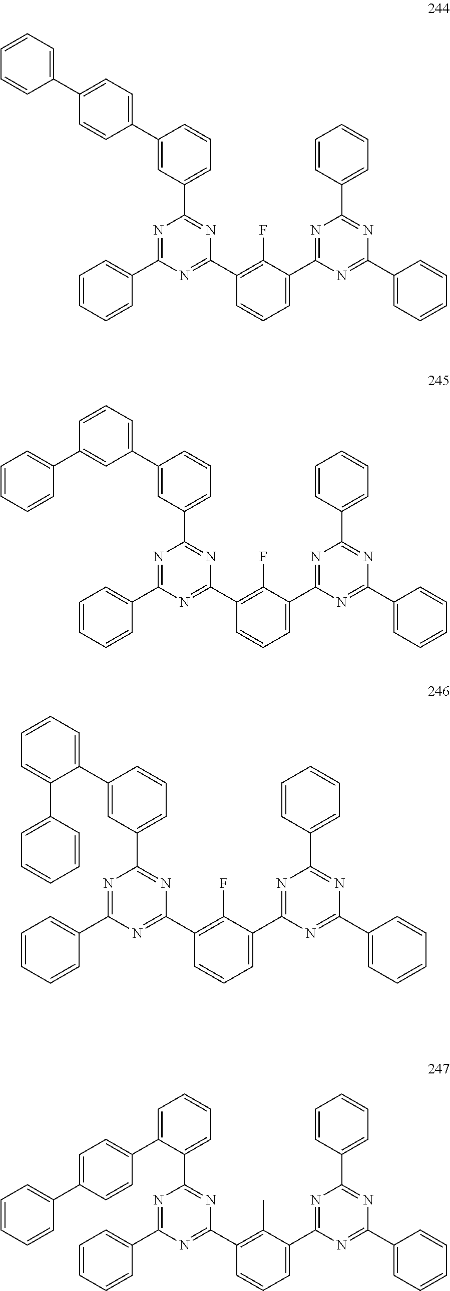 [SMILES: Fc1c(-c2nc(-c3ccccc3)nc(-c3ccccc3)n2)cccc1-c1nc(-c2ccccc2)nc(-c2cccc(-c3ccc(-c4ccccc4)cc3)c2)n1 ; Fc1c(-c2nc(-c3ccccc3)nc(-c3ccccc3)n2)cccc1-c1nc(-c2ccccc2)nc(-c2cccc(-c3cccc(-c4ccccc4)c3)c2)n1 ; Fc1c(-c2nc(-c3ccccc3)nc(-c3ccccc3)n2)cccc1-c1nc(-c2ccccc2)nc(-c2cccc(-c3ccccc3-c3ccccc3)c2)n1 ; Cc1c(-c2nc(-c3ccccc3)nc(-c3ccccc3)n2)cccc1-c1nc(-c2ccccc2)nc(-c2ccccc2-c2ccc(-c3ccccc3)cc2)n1]

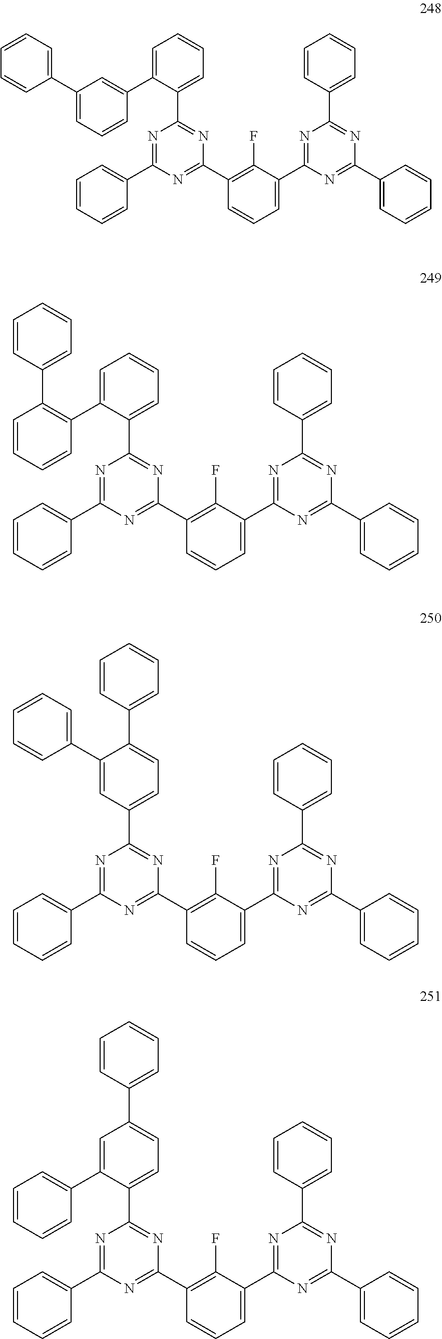 [SMILES: Fc1c(-c2nc(-c3ccccc3)nc(-c3ccccc3)n2)cccc1-c1nc(-c2ccccc2)nc(-c2ccccc2-c2cccc(-c3ccccc3)c2)n1 ; Fc1c(-c2nc(-c3ccccc3)nc(-c3ccccc3)n2)cccc1-c1nc(-c2ccccc2)nc(-c2ccccc2-c2ccccc2-c2ccccc2)n1 ; Fc1c(-c2nc(-c3ccccc3)nc(-c3ccccc3)n2)cccc1-c1nc(-c2ccccc2)nc(-c2ccc(-c3ccccc3)c(-c3ccccc3)c2)n1 ; Fc1c(-c2nc(-c3ccccc3)nc(-c3ccccc3)n2)cccc1-c1nc(-c2ccccc2)nc(-c2ccc(-c3ccccc3)cc2-c2ccccc2)n1]

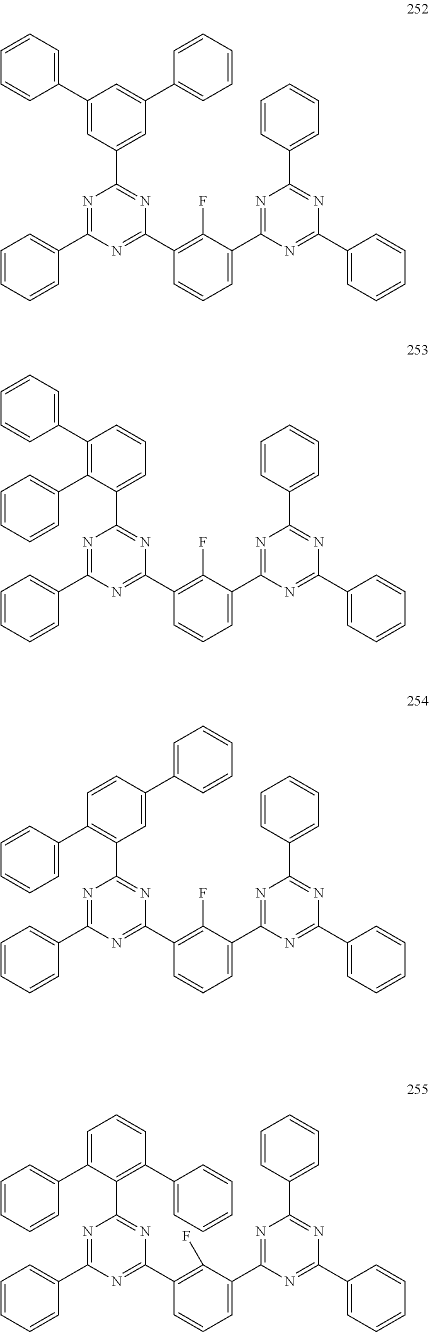 [SMILES: Fc1c(-c2nc(-c3ccccc3)nc(-c3ccccc3)n2)cccc1-c1nc(-c2ccccc2)nc(-c2cc(-c3ccccc3)cc(-c3ccccc3)c2)n1 ; Fc1c(-c2nc(-c3ccccc3)nc(-c3ccccc3)n2)cccc1-c1nc(-c2ccccc2)nc(-c2cccc(-c3ccccc3)c2-c2ccccc2)n1 ; Fc1c(-c2nc(-c3ccccc3)nc(-c3ccccc3)n2)cccc1-c1nc(-c2ccccc2)nc(-c2cc(-c3ccccc3)ccc2-c2ccccc2)n1 ; Fc1c(-c2nc(-c3ccccc3)nc(-c3ccccc3)n2)cccc1-c1nc(-c2ccccc2)nc(-c2c(-c3ccccc3)cccc2-c2ccccc2)n1]

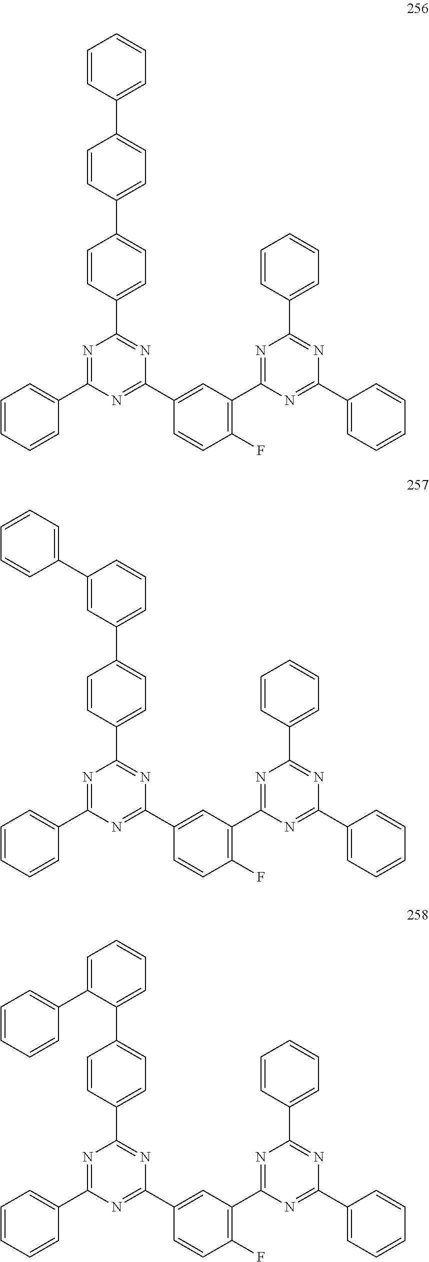 [SMILES: Fc1ccc(-c2nc(-c3ccccc3)nc(-c3ccc(-c4ccc(-c5ccccc5)cc4)cc3)n2)cc1-c1nc(-c2ccccc2)nc(-c2ccccc2)n1 ; Fc1ccc(-c2nc(-c3ccccc3)nc(-c3ccc(-c4cccc(-c5ccccc5)c4)cc3)n2)cc1-c1nc(-c2ccccc2)nc(-c2ccccc2)n1 ; Fc1ccc(-c2nc(-c3ccccc3)nc(-c3ccc(-c4ccccc4-c4ccccc4)cc3)n2)cc1-c1nc(-c2ccccc2)nc(-c2ccccc2)n1]

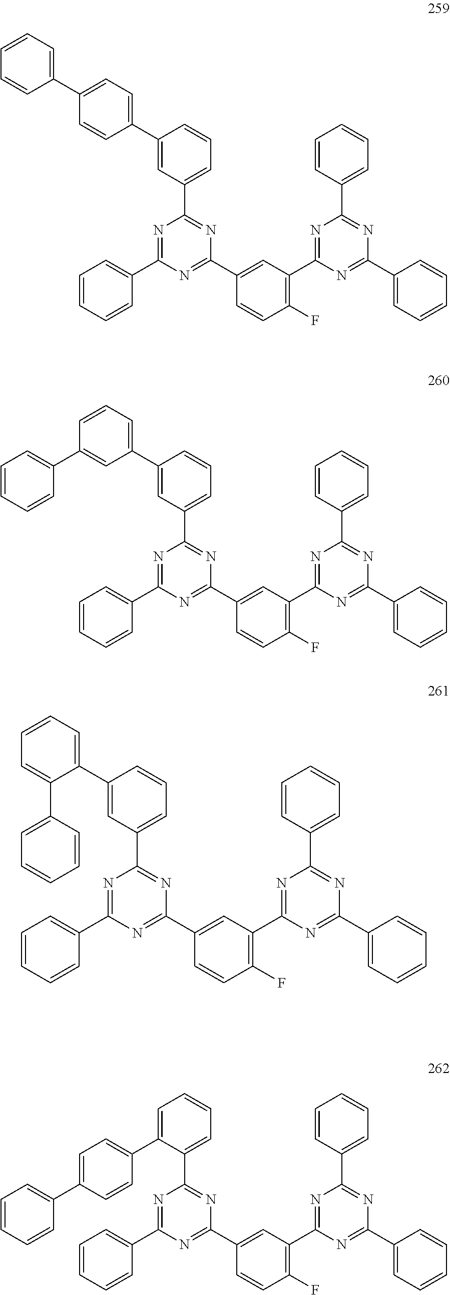 [SMILES: Fc1ccc(-c2nc(-c3ccccc3)nc(-c3cccc(-c4ccc(-c5ccccc5)cc4)c3)n2)cc1-c1nc(-c2ccccc2)nc(-c2ccccc2)n1 ; Fc1ccc(-c2nc(-c3ccccc3)nc(-c3cccc(-c4cccc(-c5ccccc5)c4)c3)n2)cc1-c1nc(-c2ccccc2)nc(-c2ccccc2)n1 ; Fc1ccc(-c2nc(-c3ccccc3)nc(-c3cccc(-c4ccccc4-c4ccccc4)c3)n2)cc1-c1nc(-c2ccccc2)nc(-c2ccccc2)n1 ; Fc1ccc(-c2nc(-c3ccccc3)nc(-c3ccccc3-c3ccc(-c4ccccc4)cc3)n2)cc1-c1nc(-c2ccccc2)nc(-c2ccccc2)n1]

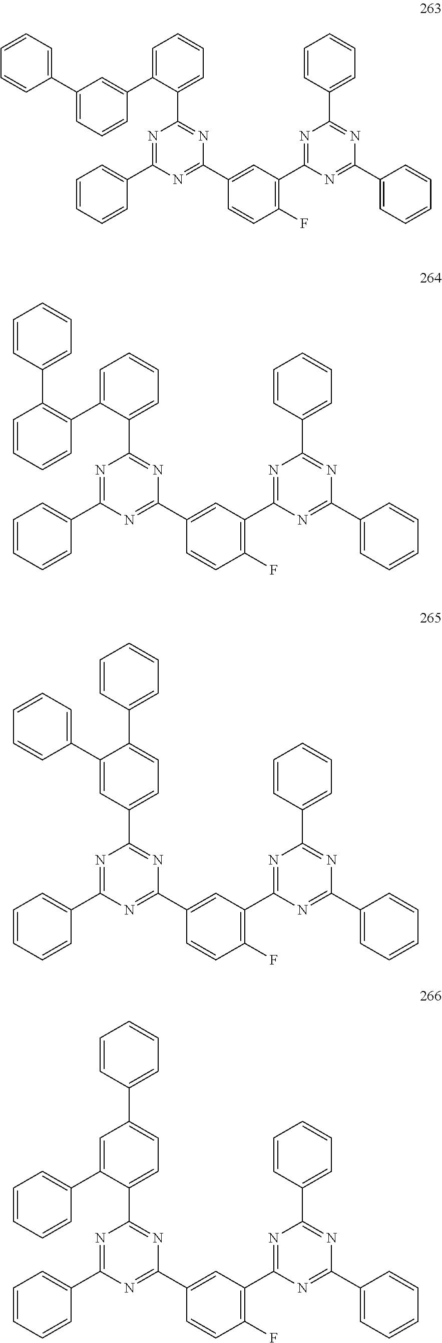 [SMILES: Fc1ccc(-c2nc(-c3ccccc3)nc(-c3ccccc3-c3cccc(-c4ccccc4)c3)n2)cc1-c1nc(-c2ccccc2)nc(-c2ccccc2)n1 ; Fc1ccc(-c2nc(-c3ccccc3)nc(-c3ccccc3-c3ccccc3-c3ccccc3)n2)cc1-c1nc(-c2ccccc2)nc(-c2ccccc2)n1 ; Fc1ccc(-c2nc(-c3ccccc3)nc(-c3ccc(-c4ccccc4)c(-c4ccccc4)c3)n2)cc1-c1nc(-c2ccccc2)nc(-c2ccccc2)n1 ; Fc1ccc(-c2nc(-c3ccccc3)nc(-c3ccc(-c4ccccc4)cc3-c3ccccc3)n2)cc1-c1nc(-c2ccccc2)nc(-c2ccccc2)n1]

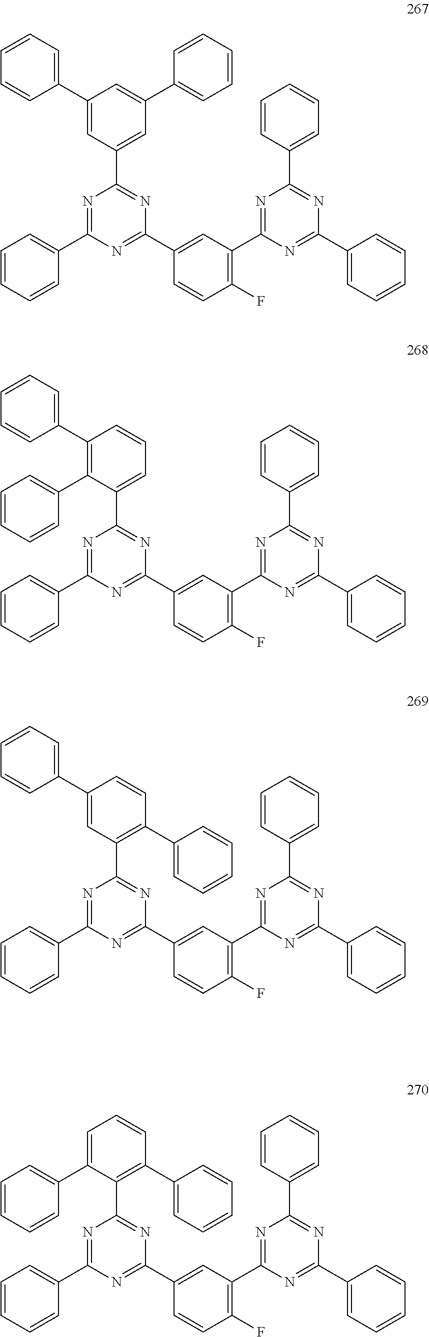 [SMILES: Fc1ccc(-c2nc(-c3ccccc3)nc(-c3cc(-c4ccccc4)cc(-c4ccccc4)c3)n2)cc1-c1nc(-c2ccccc2)nc(-c2ccccc2)n1 ; Fc1ccc(-c2nc(-c3ccccc3)nc(-c3cccc(-c4ccccc4)c3-c3ccccc3)n2)cc1-c1nc(-c2ccccc2)nc(-c2ccccc2)n1 ; Fc1ccc(-c2nc(-c3ccccc3)nc(-c3cc(-c4ccccc4)ccc3-c3ccccc3)n2)cc1-c1nc(-c2ccccc2)nc(-c2ccccc2)n1 ; Fc1ccc(-c2nc(-c3ccccc3)nc(-c3c(-c4ccccc4)cccc3-c3ccccc3)n2)cc1-c1nc(-c2ccccc2)nc(-c2ccccc2)n1]

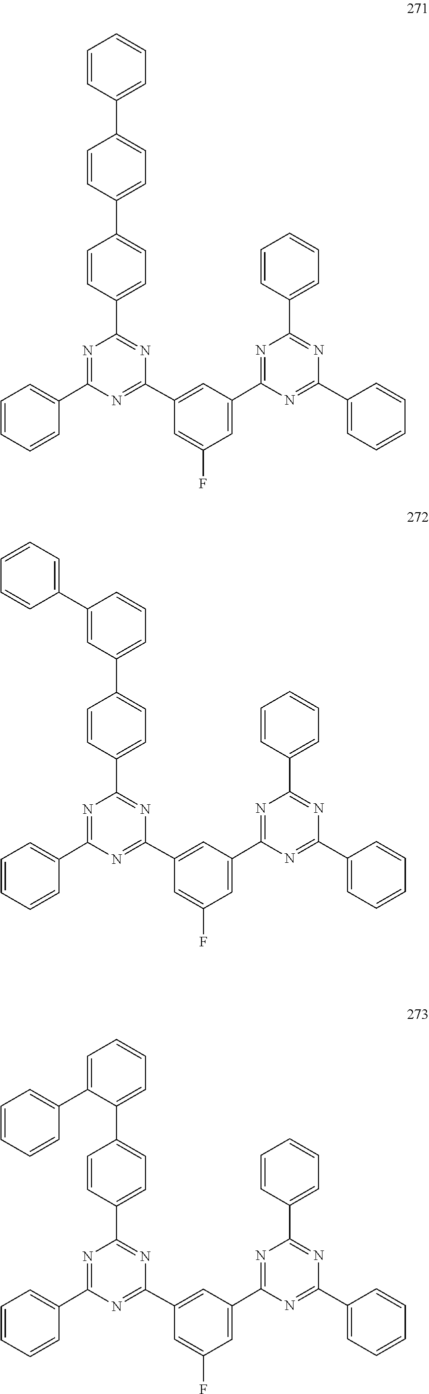 [SMILES: Fc1cc(-c2nc(-c3ccccc3)nc(-c3ccccc3)n2)cc(-c2nc(-c3ccccc3)nc(-c3ccc(-c4ccc(-c5ccccc5)cc4)cc3)n2)c1 ; Fc1cc(-c2nc(-c3ccccc3)nc(-c3ccccc3)n2)cc(-c2nc(-c3ccccc3)nc(-c3ccc(-c4cccc(-c5ccccc5)c4)cc3)n2)c1 ; Fc1cc(-c2nc(-c3ccccc3)nc(-c3ccccc3)n2)cc(-c2nc(-c3ccccc3)nc(-c3ccc(-c4ccccc4-c4ccccc4)cc3)n2)c1]

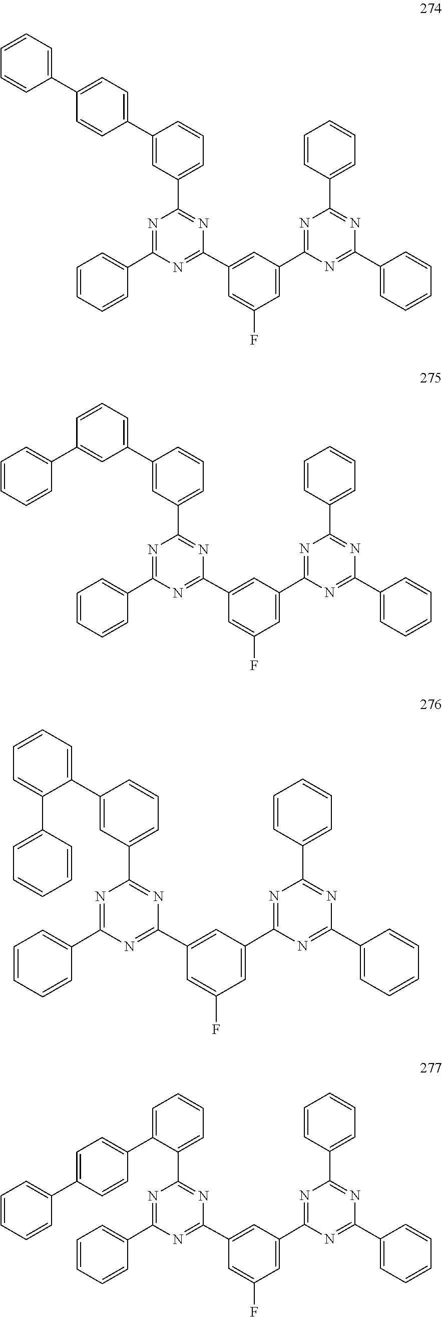 [SMILES: Fc1cc(-c2nc(-c3ccccc3)nc(-c3ccccc3)n2)cc(-c2nc(-c3ccccc3)nc(-c3cccc(-c4ccc(-c5ccccc5)cc4)c3)n2)c1 ; Fc1cc(-c2nc(-c3ccccc3)nc(-c3ccccc3)n2)cc(-c2nc(-c3ccccc3)nc(-c3cccc(-c4cccc(-c5ccccc5)c4)c3)n2)c1 ; Fc1cc(-c2nc(-c3ccccc3)nc(-c3ccccc3)n2)cc(-c2nc(-c3ccccc3)nc(-c3cccc(-c4ccccc4-c4ccccc4)c3)n2)c1 ; Fc1cc(-c2nc(-c3ccccc3)nc(-c3ccccc3)n2)cc(-c2nc(-c3ccccc3)nc(-c3ccccc3-c3ccc(-c4ccccc4)cc3)n2)c1]

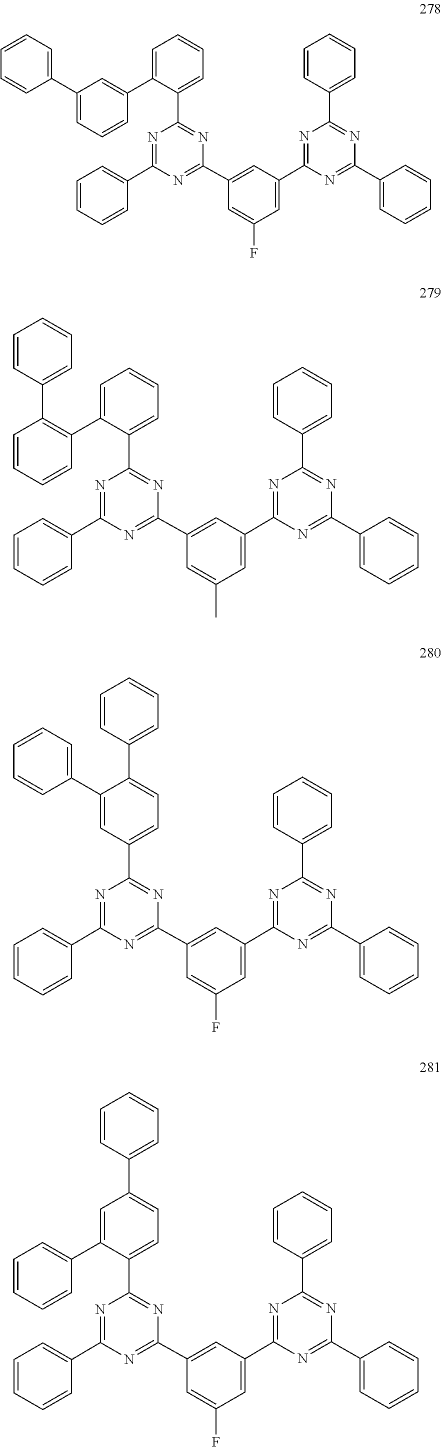 [SMILES: Fc1cc(-c2nc(-c3ccccc3)nc(-c3ccccc3)n2)cc(-c2nc(-c3ccccc3)nc(-c3ccccc3-c3cccc(-c4ccccc4)c3)n2)c1 ; Cc1cc(-c2nc(-c3ccccc3)nc(-c3ccccc3)n2)cc(-c2nc(-c3ccccc3)nc(-c3ccccc3-c3ccccc3-c3ccccc3)n2)c1 ; Fc1cc(-c2nc(-c3ccccc3)nc(-c3ccccc3)n2)cc(-c2nc(-c3ccccc3)nc(-c3ccc(-c4ccccc4)c(-c4ccccc4)c3)n2)c1 ; Fc1cc(-c2nc(-c3ccccc3)nc(-c3ccccc3)n2)cc(-c2nc(-c3ccccc3)nc(-c3ccc(-c4ccccc4)cc3-c3ccccc3)n2)c1]

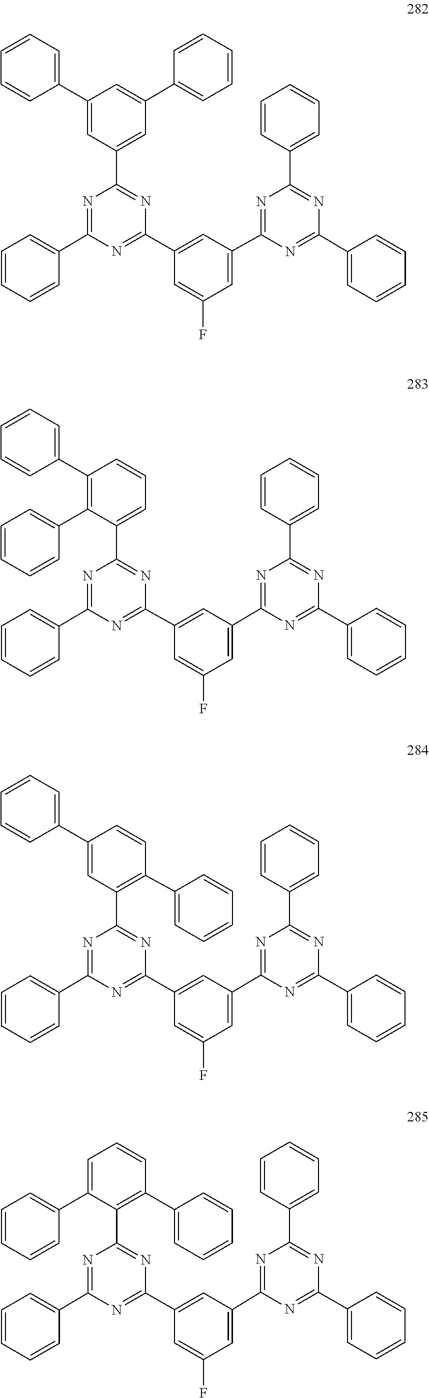 [SMILES: Fc1cc(-c2nc(-c3ccccc3)nc(-c3ccccc3)n2)cc(-c2nc(-c3ccccc3)nc(-c3cc(-c4ccccc4)cc(-c4ccccc4)c3)n2)c1 ; Fc1cc(-c2nc(-c3ccccc3)nc(-c3ccccc3)n2)cc(-c2nc(-c3ccccc3)nc(-c3cccc(-c4ccccc4)c3-c3ccccc3)n2)c1 ; Fc1cc(-c2nc(-c3ccccc3)nc(-c3ccccc3)n2)cc(-c2nc(-c3ccccc3)nc(-c3cc(-c4ccccc4)ccc3-c3ccccc3)n2)c1 ; Fc1cc(-c2nc(-c3ccccc3)nc(-c3ccccc3)n2)cc(-c2nc(-c3ccccc3)nc(-c3c(-c4ccccc4)cccc3-c3ccccc3)n2)c1]

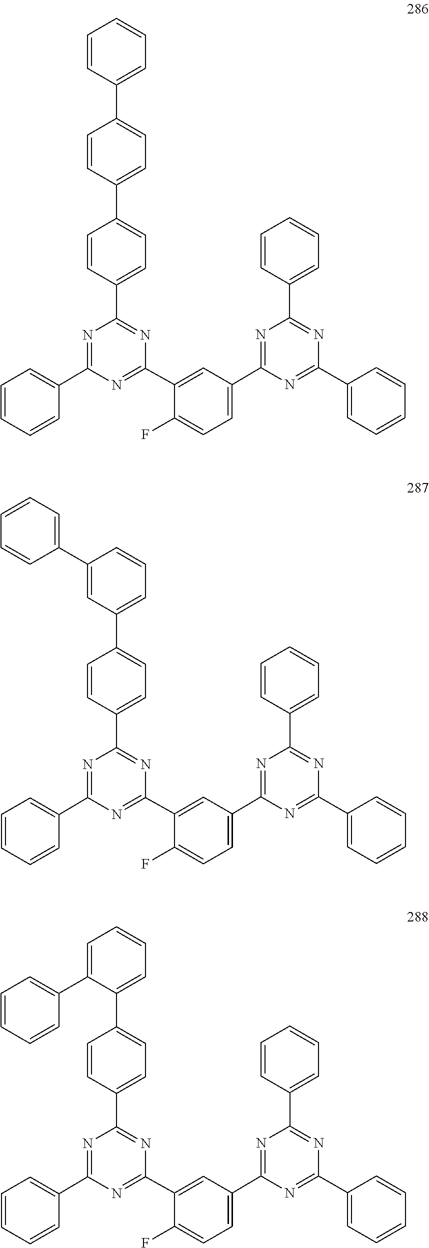 [SMILES: Fc1ccc(-c2nc(-c3ccccc3)nc(-c3ccccc3)n2)cc1-c1nc(-c2ccccc2)nc(-c2ccc(-c3ccc(-c4ccccc4)cc3)cc2)n1 ; Fc1ccc(-c2nc(-c3ccccc3)nc(-c3ccccc3)n2)cc1-c1nc(-c2ccccc2)nc(-c2ccc(-c3cccc(-c4ccccc4)c3)cc2)n1 ; Fc1ccc(-c2nc(-c3ccccc3)nc(-c3ccccc3)n2)cc1-c1nc(-c2ccccc2)nc(-c2ccc(-c3ccccc3-c3ccccc3)cc2)n1]

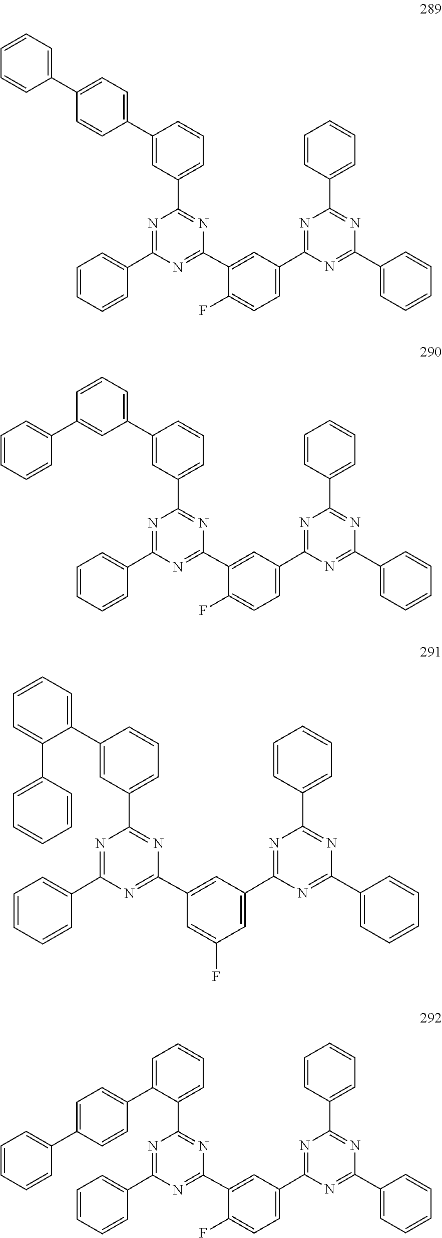 [SMILES: Fc1ccc(-c2nc(-c3ccccc3)nc(-c3ccccc3)n2)cc1-c1nc(-c2ccccc2)nc(-c2cccc(-c3ccc(-c4ccccc4)cc3)c2)n1 ; Fc1ccc(-c2nc(-c3ccccc3)nc(-c3ccccc3)n2)cc1-c1nc(-c2ccccc2)nc(-c2cccc(-c3cccc(-c4ccccc4)c3)c2)n1 ; Fc1cc(-c2nc(-c3ccccc3)nc(-c3ccccc3)n2)cc(-c2nc(-c3ccccc3)nc(-c3cccc(-c4ccccc4-c4ccccc4)c3)n2)c1 ; Fc1ccc(-c2nc(-c3ccccc3)nc(-c3ccccc3)n2)cc1-c1nc(-c2ccccc2)nc(-c2ccccc2-c2ccc(-c3ccccc3)cc2)n1]

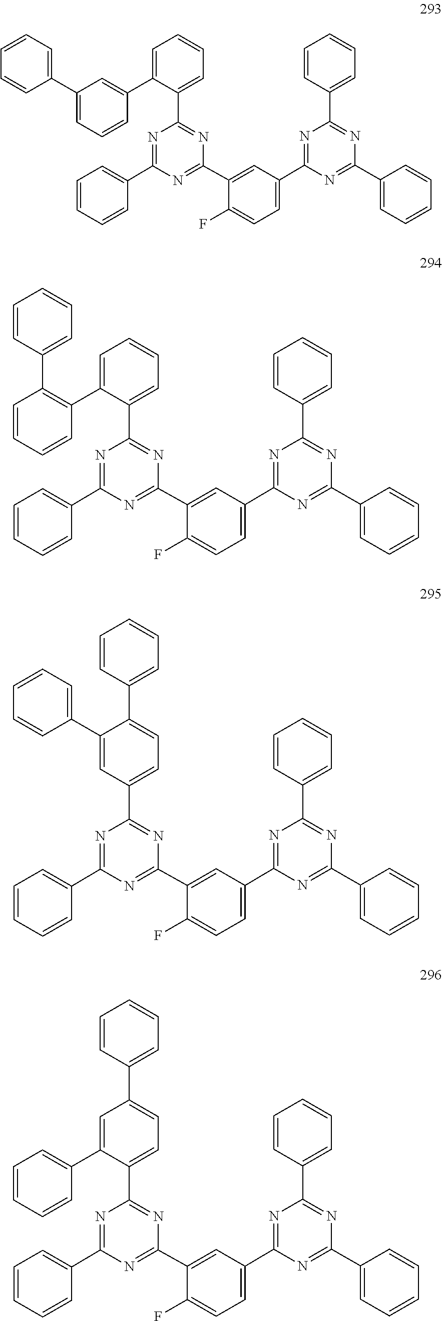 [SMILES: Fc1ccc(-c2nc(-c3ccccc3)nc(-c3ccccc3)n2)cc1-c1nc(-c2ccccc2)nc(-c2ccccc2-c2cccc(-c3ccccc3)c2)n1 ; Fc1ccc(-c2nc(-c3ccccc3)nc(-c3ccccc3)n2)cc1-c1nc(-c2ccccc2)nc(-c2ccccc2-c2ccccc2-c2ccccc2)n1 ; Fc1ccc(-c2nc(-c3ccccc3)nc(-c3ccccc3)n2)cc1-c1nc(-c2ccccc2)nc(-c2ccc(-c3ccccc3)c(-c3ccccc3)c2)n1 ; Fc1ccc(-c2nc(-c3ccccc3)nc(-c3ccccc3)n2)cc1-c1nc(-c2ccccc2)nc(-c2ccc(-c3ccccc3)cc2-c2ccccc2)n1]

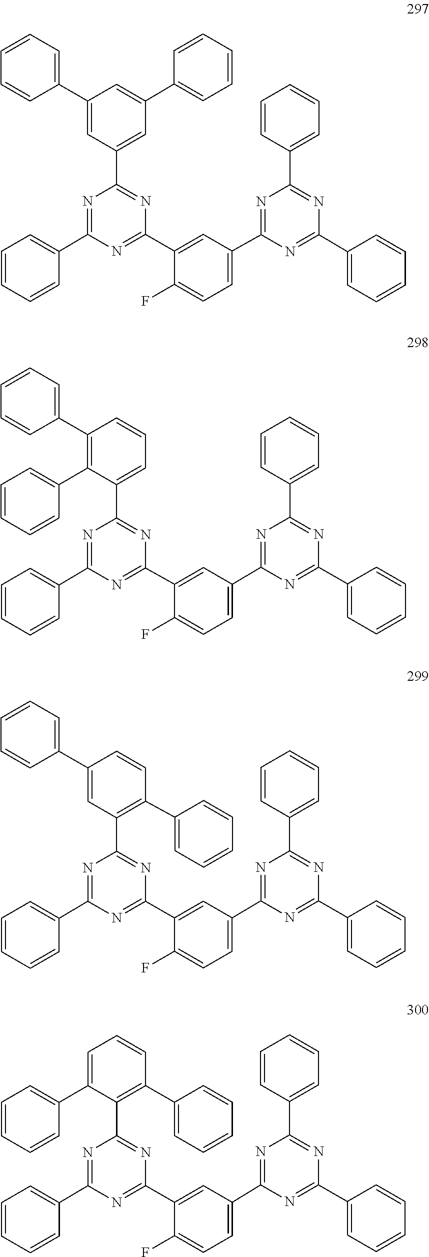 [SMILES: Fc1ccc(-c2nc(-c3ccccc3)nc(-c3ccccc3)n2)cc1-c1nc(-c2ccccc2)nc(-c2cc(-c3ccccc3)cc(-c3ccccc3)c2)n1 ; Fc1ccc(-c2nc(-c3ccccc3)nc(-c3ccccc3)n2)cc1-c1nc(-c2ccccc2)nc(-c2cccc(-c3ccccc3)c2-c2ccccc2)n1 ; Fc1ccc(-c2nc(-c3ccccc3)nc(-c3ccccc3)n2)cc1-c1nc(-c2ccccc2)nc(-c2cc(-c3ccccc3)ccc2-c2ccccc2)n1 ; Fc1ccc(-c2nc(-c3ccccc3)nc(-c3ccccc3)n2)cc1-c1nc(-c2ccccc2)nc(-c2c(-c3ccccc3)cccc2-c2ccccc2)n1]

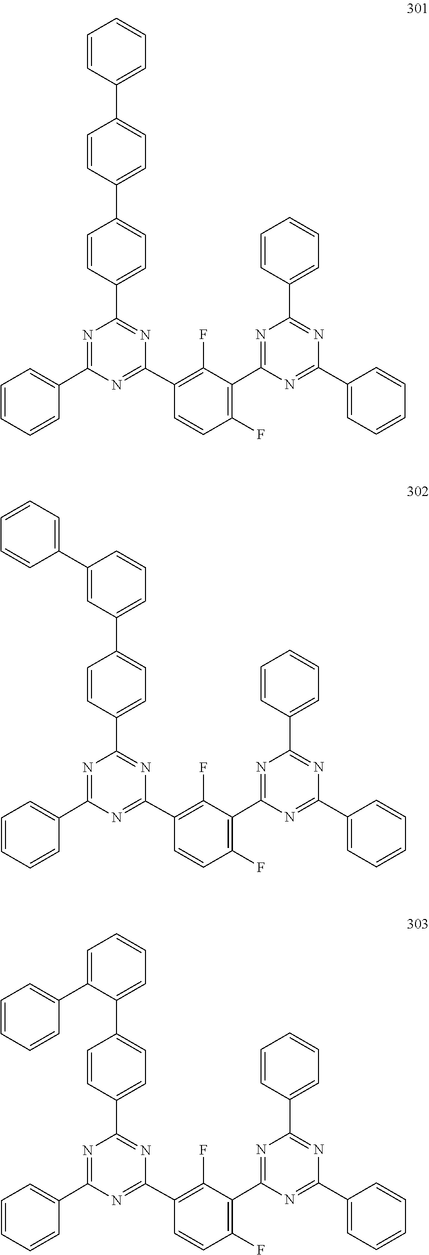 [SMILES: Fc1ccc(-c2nc(-c3ccccc3)nc(-c3ccc(-c4ccc(-c5ccccc5)cc4)cc3)n2)c(F)c1-c1nc(-c2ccccc2)nc(-c2ccccc2)n1 ; Fc1ccc(-c2nc(-c3ccccc3)nc(-c3ccc(-c4cccc(-c5ccccc5)c4)cc3)n2)c(F)c1-c1nc(-c2ccccc2)nc(-c2ccccc2)n1 ; Fc1ccc(-c2nc(-c3ccccc3)nc(-c3ccc(-c4ccccc4-c4ccccc4)cc3)n2)c(F)c1-c1nc(-c2ccccc2)nc(-c2ccccc2)n1]

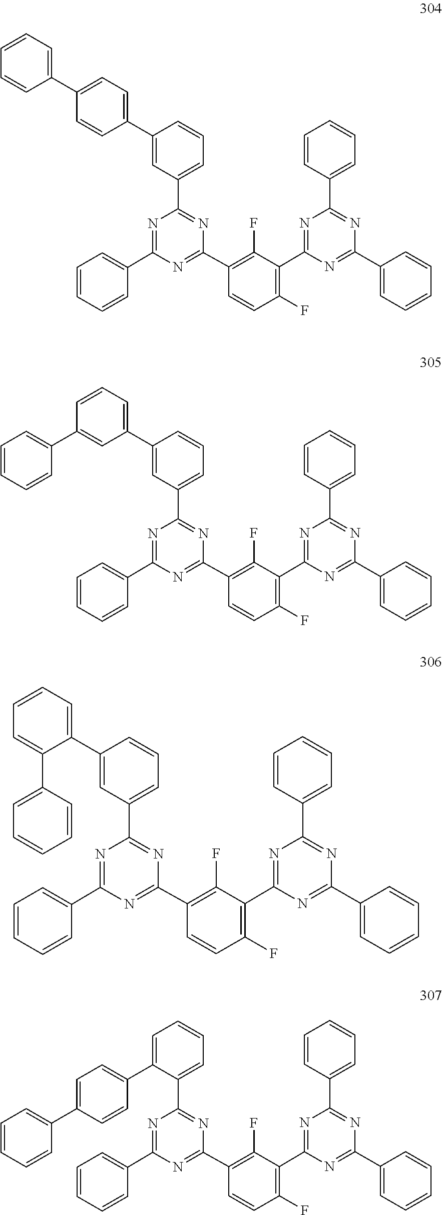 [SMILES: Fc1ccc(-c2nc(-c3ccccc3)nc(-c3cccc(-c4ccc(-c5ccccc5)cc4)c3)n2)c(F)c1-c1nc(-c2ccccc2)nc(-c2ccccc2)n1 ; Fc1ccc(-c2nc(-c3ccccc3)nc(-c3cccc(-c4cccc(-c5ccccc5)c4)c3)n2)c(F)c1-c1nc(-c2ccccc2)nc(-c2ccccc2)n1 ; Fc1ccc(-c2nc(-c3ccccc3)nc(-c3cccc(-c4ccccc4-c4ccccc4)c3)n2)c(F)c1-c1nc(-c2ccccc2)nc(-c2ccccc2)n1 ; Fc1ccc(-c2nc(-c3ccccc3)nc(-c3ccccc3-c3ccc(-c4ccccc4)cc3)n2)c(F)c1-c1nc(-c2ccccc2)nc(-c2ccccc2)n1]

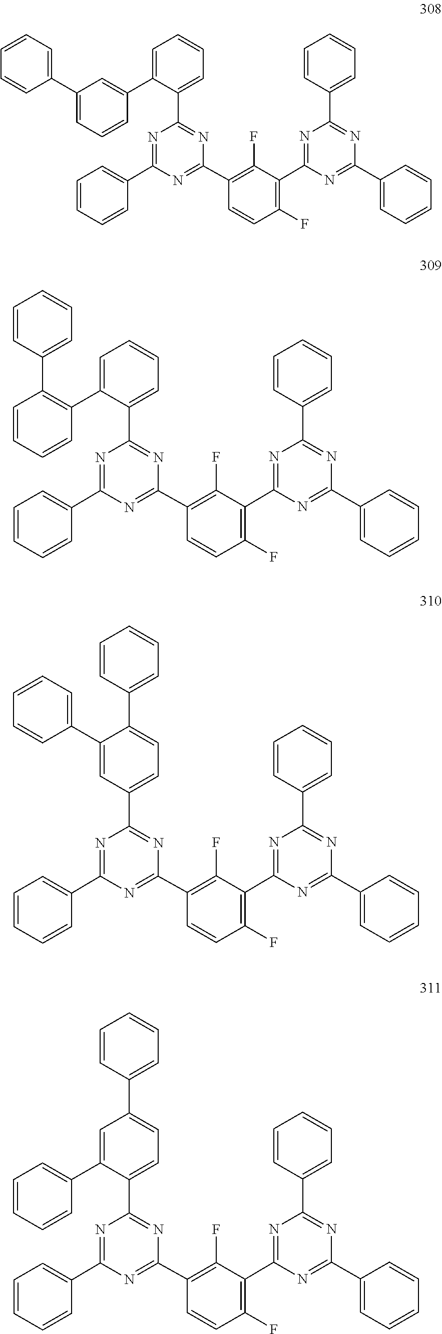 [SMILES: Fc1ccc(-c2nc(-c3ccccc3)nc(-c3ccccc3-c3cccc(-c4ccccc4)c3)n2)c(F)c1-c1nc(-c2ccccc2)nc(-c2ccccc2)n1 ; Fc1ccc(-c2nc(-c3ccccc3)nc(-c3ccccc3-c3ccccc3-c3ccccc3)n2)c(F)c1-c1nc(-c2ccccc2)nc(-c2ccccc2)n1 ; Fc1ccc(-c2nc(-c3ccccc3)nc(-c3ccc(-c4ccccc4)c(-c4ccccc4)c3)n2)c(F)c1-c1nc(-c2ccccc2)nc(-c2ccccc2)n1 ; Fc1ccc(-c2nc(-c3ccccc3)nc(-c3ccc(-c4ccccc4)cc3-c3ccccc3)n2)c(F)c1-c1nc(-c2ccccc2)nc(-c2ccccc2)n1]

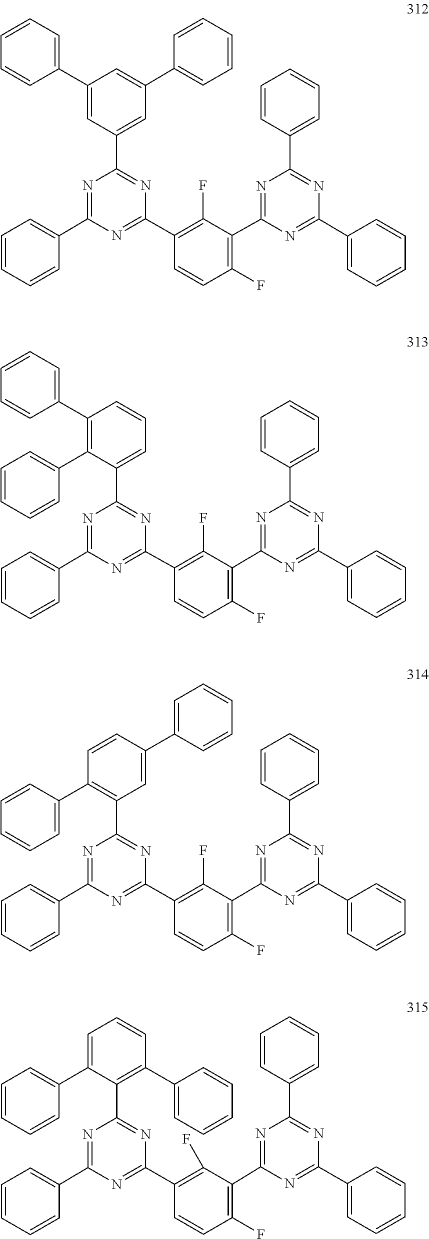 [SMILES: Fc1ccc(-c2nc(-c3ccccc3)nc(-c3cc(-c4ccccc4)cc(-c4ccccc4)c3)n2)c(F)c1-c1nc(-c2ccccc2)nc(-c2ccccc2)n1 ; Fc1ccc(-c2nc(-c3ccccc3)nc(-c3cccc(-c4ccccc4)c3-c3ccccc3)n2)c(F)c1-c1nc(-c2ccccc2)nc(-c2ccccc2)n1 ; Fc1ccc(-c2nc(-c3ccccc3)nc(-c3cc(-c4ccccc4)ccc3-c3ccccc3)n2)c(F)c1-c1nc(-c2ccccc2)nc(-c2ccccc2)n1 ; Fc1ccc(-c2nc(-c3ccccc3)nc(-c3c(-c4ccccc4)cccc3-c3ccccc3)n2)c(F)c1-c1nc(-c2ccccc2)nc(-c2ccccc2)n1]

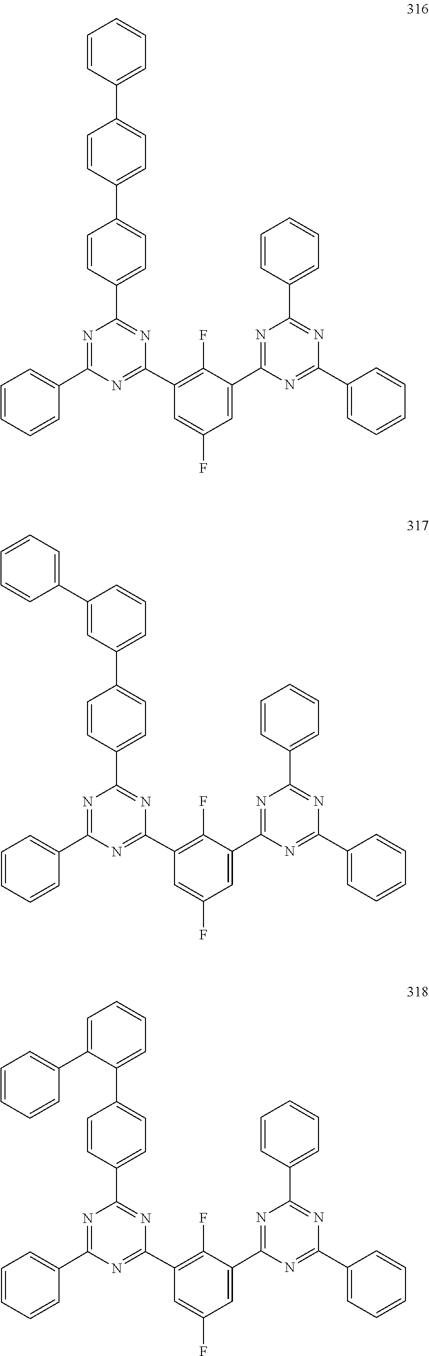 [SMILES: Fc1cc(-c2nc(-c3ccccc3)nc(-c3ccccc3)n2)c(F)c(-c2nc(-c3ccccc3)nc(-c3ccc(-c4ccc(-c5ccccc5)cc4)cc3)n2)c1 ; Fc1cc(-c2nc(-c3ccccc3)nc(-c3ccccc3)n2)c(F)c(-c2nc(-c3ccccc3)nc(-c3ccc(-c4cccc(-c5ccccc5)c4)cc3)n2)c1 ; Fc1cc(-c2nc(-c3ccccc3)nc(-c3ccccc3)n2)c(F)c(-c2nc(-c3ccccc3)nc(-c3ccc(-c4ccccc4-c4ccccc4)cc3)n2)c1]

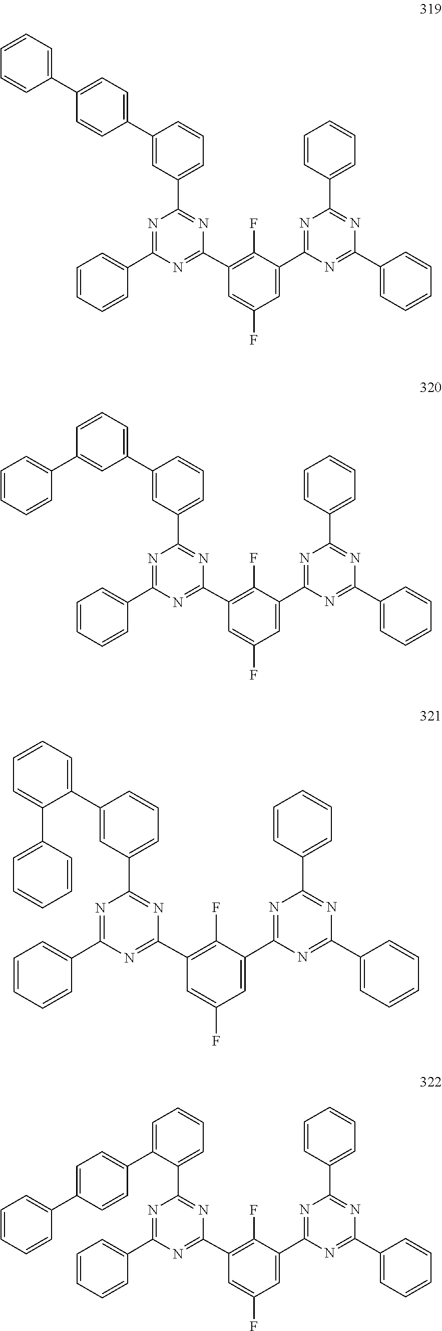 [SMILES: Fc1cc(-c2nc(-c3ccccc3)nc(-c3ccccc3)n2)c(F)c(-c2nc(-c3ccccc3)nc(-c3cccc(-c4ccc(-c5ccccc5)cc4)c3)n2)c1 ; Fc1cc(-c2nc(-c3ccccc3)nc(-c3ccccc3)n2)c(F)c(-c2nc(-c3ccccc3)nc(-c3cccc(-c4cccc(-c5ccccc5)c4)c3)n2)c1 ; Fc1cc(-c2nc(-c3ccccc3)nc(-c3ccccc3)n2)c(F)c(-c2nc(-c3ccccc3)nc(-c3cccc(-c4ccccc4-c4ccccc4)c3)n2)c1 ; Fc1cc(-c2nc(-c3ccccc3)nc(-c3ccccc3)n2)c(F)c(-c2nc(-c3ccccc3)nc(-c3ccccc3-c3ccc(-c4ccccc4)cc3)n2)c1]

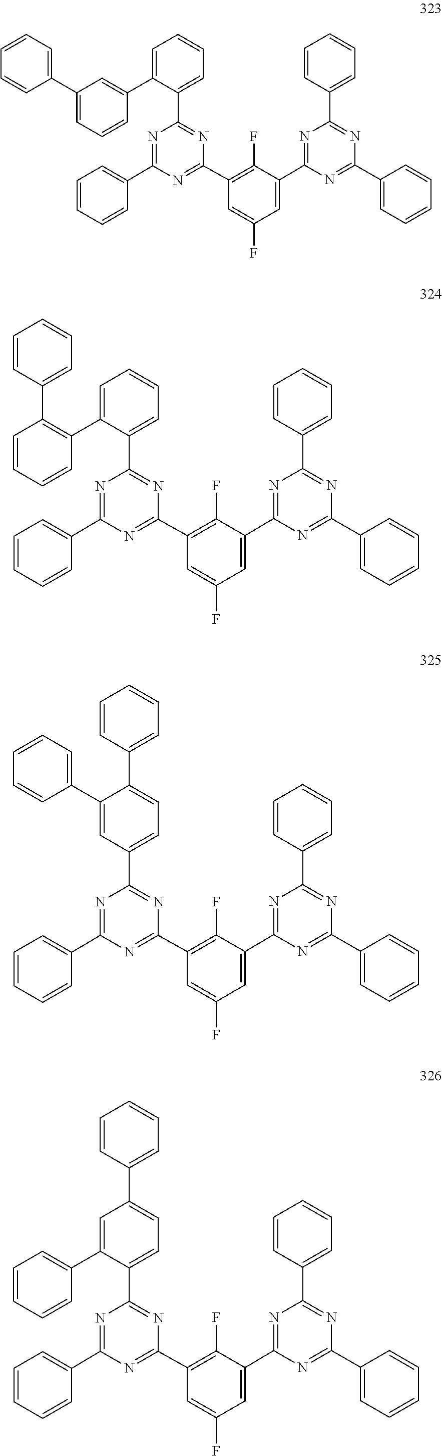 [SMILES: Fc1cc(-c2nc(-c3ccccc3)nc(-c3ccccc3)n2)c(F)c(-c2nc(-c3ccccc3)nc(-c3ccccc3-c3cccc(-c4ccccc4)c3)n2)c1 ; Fc1cc(-c2nc(-c3ccccc3)nc(-c3ccccc3)n2)c(F)c(-c2nc(-c3ccccc3)nc(-c3ccccc3-c3ccccc3-c3ccccc3)n2)c1 ; Fc1cc(-c2nc(-c3ccccc3)nc(-c3ccccc3)n2)c(F)c(-c2nc(-c3ccccc3)nc(-c3ccc(-c4ccccc4)c(-c4ccccc4)c3)n2)c1 ; Fc1cc(-c2nc(-c3ccccc3)nc(-c3ccccc3)n2)c(F)c(-c2nc(-c3ccccc3)nc(-c3ccc(-c4ccccc4)cc3-c3ccccc3)n2)c1]

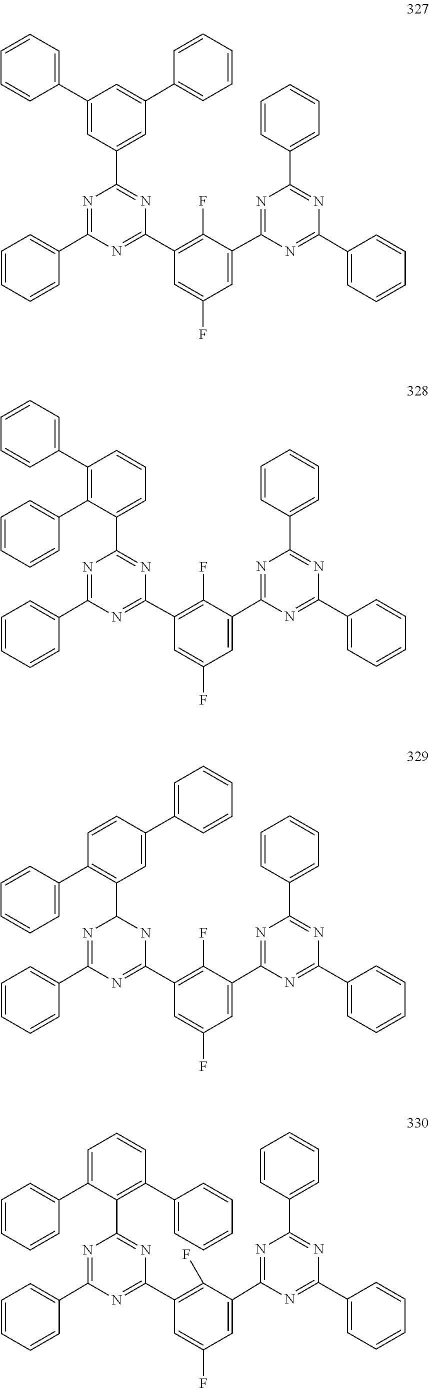 [SMILES: Fc1cc(-c2nc(-c3ccccc3)nc(-c3ccccc3)n2)c(F)c(-c2nc(-c3ccccc3)nc(-c3cc(-c4ccccc4)cc(-c4ccccc4)c3)n2)c1 ; Fc1cc(-c2nc(-c3ccccc3)nc(-c3ccccc3)n2)c(F)c(-c2nc(-c3ccccc3)nc(-c3cccc(-c4ccccc4)c3-c3ccccc3)n2)c1 ; Fc1cc(C2=NC(c3ccccc3)=NC(c3cc(-c4ccccc4)ccc3-c3ccccc3)N2)c(F)c(-c2nc(-c3ccccc3)nc(-c3ccccc3)n2)c1 ; Fc1cc(-c2nc(-c3ccccc3)nc(-c3ccccc3)n2)c(F)c(-c2nc(-c3ccccc3)nc(-c3c(-c4ccccc4)cccc3-c3ccccc3)n2)c1]

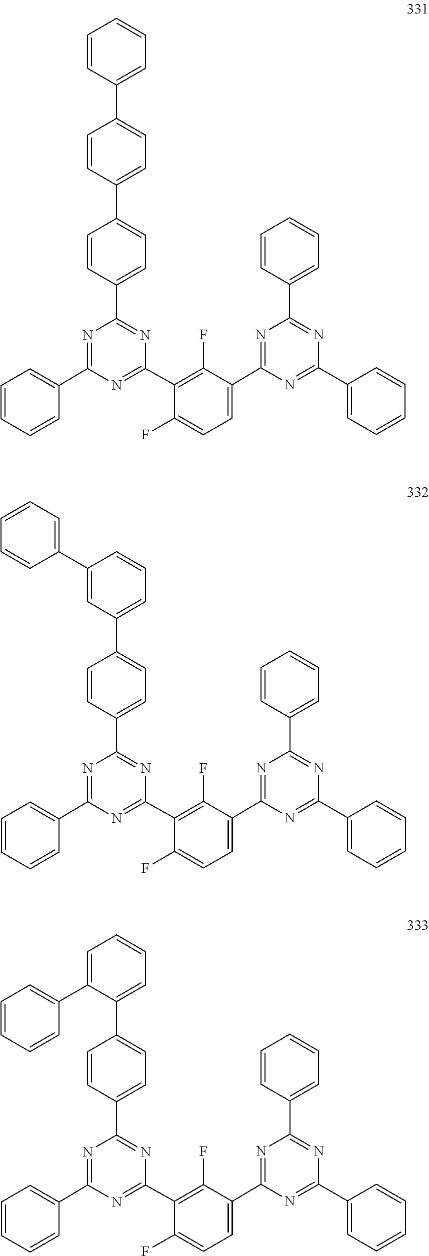 [SMILES: Fc1ccc(-c2nc(-c3ccccc3)nc(-c3ccccc3)n2)c(F)c1-c1nc(-c2ccccc2)nc(-c2ccc(-c3ccc(-c4ccccc4)cc3)cc2)n1 ; Fc1ccc(-c2nc(-c3ccccc3)nc(-c3ccccc3)n2)c(F)c1-c1nc(-c2ccccc2)nc(-c2ccc(-c3cccc(-c4ccccc4)c3)cc2)n1 ; Fc1ccc(-c2nc(-c3ccccc3)nc(-c3ccccc3)n2)c(F)c1-c1nc(-c2ccccc2)nc(-c2ccc(-c3ccccc3-c3ccccc3)cc2)n1]

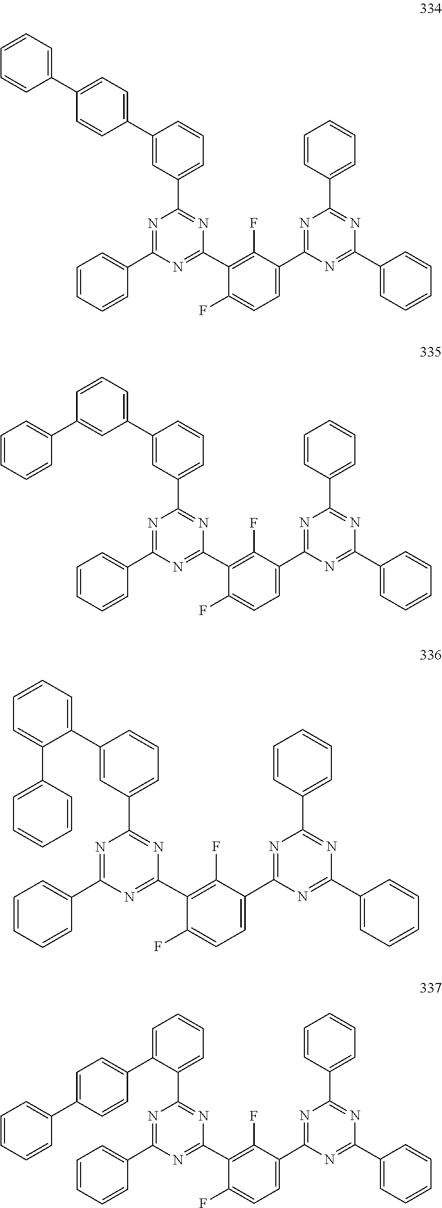 [SMILES: Fc1ccc(-c2nc(-c3ccccc3)nc(-c3ccccc3)n2)c(F)c1-c1nc(-c2ccccc2)nc(-c2cccc(-c3ccc(-c4ccccc4)cc3)c2)n1 ; Fc1ccc(-c2nc(-c3ccccc3)nc(-c3ccccc3)n2)c(F)c1-c1nc(-c2ccccc2)nc(-c2cccc(-c3cccc(-c4ccccc4)c3)c2)n1 ; Fc1ccc(-c2nc(-c3ccccc3)nc(-c3ccccc3)n2)c(F)c1-c1nc(-c2ccccc2)nc(-c2cccc(-c3ccccc3-c3ccccc3)c2)n1 ; Fc1ccc(-c2nc(-c3ccccc3)nc(-c3ccccc3)n2)c(F)c1-c1nc(-c2ccccc2)nc(-c2ccccc2-c2ccc(-c3ccccc3)cc2)n1]

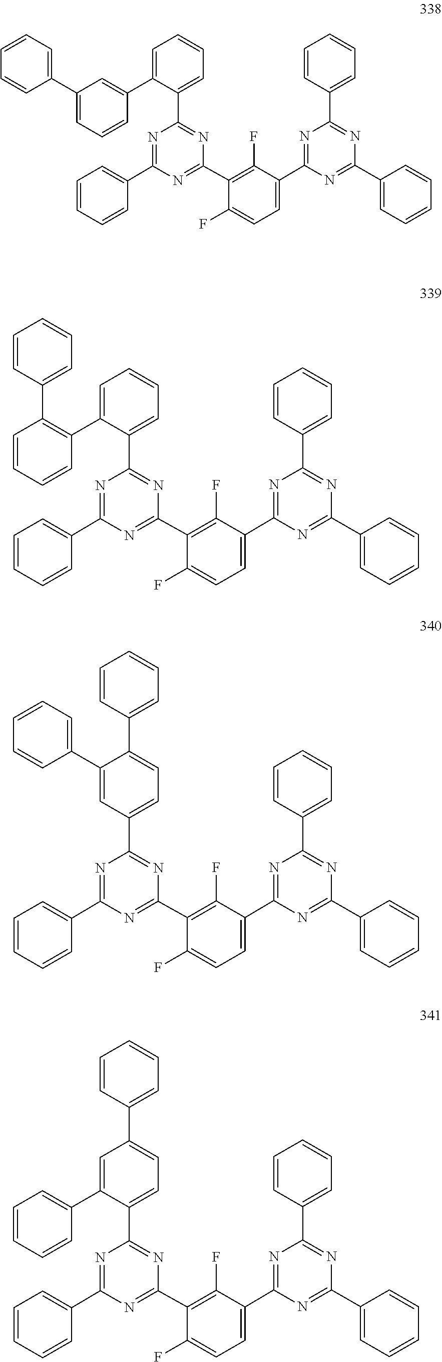 [SMILES: Fc1ccc(-c2nc(-c3ccccc3)nc(-c3ccccc3)n2)c(F)c1-c1nc(-c2ccccc2)nc(-c2ccccc2-c2cccc(-c3ccccc3)c2)n1 ; Fc1ccc(-c2nc(-c3ccccc3)nc(-c3ccccc3)n2)c(F)c1-c1nc(-c2ccccc2)nc(-c2ccccc2-c2ccccc2-c2ccccc2)n1 ; Fc1ccc(-c2nc(-c3ccccc3)nc(-c3ccccc3)n2)c(F)c1-c1nc(-c2ccccc2)nc(-c2ccc(-c3ccccc3)c(-c3ccccc3)c2)n1 ; Fc1ccc(-c2nc(-c3ccccc3)nc(-c3ccccc3)n2)c(F)c1-c1nc(-c2ccccc2)nc(-c2ccc(-c3ccccc3)cc2-c2ccccc2)n1]

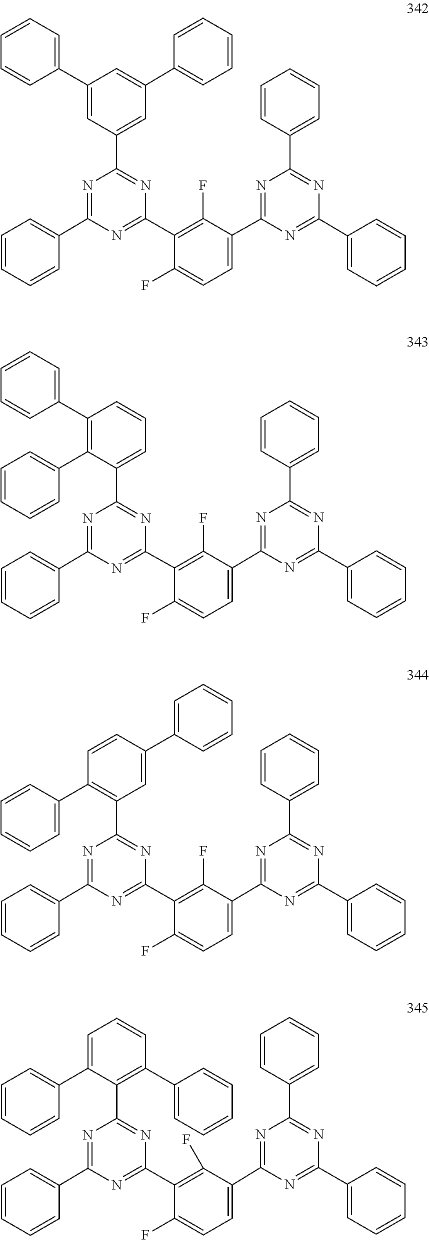 [SMILES: Fc1ccc(-c2nc(-c3ccccc3)nc(-c3ccccc3)n2)c(F)c1-c1nc(-c2ccccc2)nc(-c2cc(-c3ccccc3)cc(-c3ccccc3)c2)n1 ; Fc1ccc(-c2nc(-c3ccccc3)nc(-c3ccccc3)n2)c(F)c1-c1nc(-c2ccccc2)nc(-c2cccc(-c3ccccc3)c2-c2ccccc2)n1 ; Fc1ccc(-c2nc(-c3ccccc3)nc(-c3ccccc3)n2)c(F)c1-c1nc(-c2ccccc2)nc(-c2cc(-c3ccccc3)ccc2-c2ccccc2)n1 ; Fc1ccc(-c2nc(-c3ccccc3)nc(-c3ccccc3)n2)c(F)c1-c1nc(-c2ccccc2)nc(-c2c(-c3ccccc3)cccc2-c2ccccc2)n1]

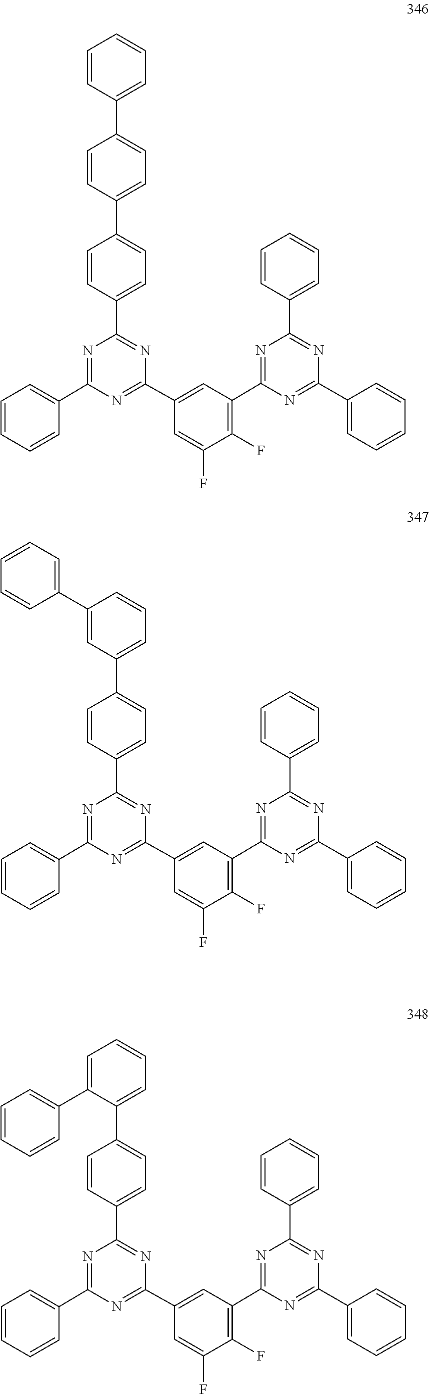 [SMILES: Fc1cc(-c2nc(-c3ccccc3)nc(-c3ccc(-c4ccc(-c5ccccc5)cc4)cc3)n2)cc(-c2nc(-c3ccccc3)nc(-c3ccccc3)n2)c1F ; Fc1cc(-c2nc(-c3ccccc3)nc(-c3ccc(-c4cccc(-c5ccccc5)c4)cc3)n2)cc(-c2nc(-c3ccccc3)nc(-c3ccccc3)n2)c1F ; Fc1cc(-c2nc(-c3ccccc3)nc(-c3ccc(-c4ccccc4-c4ccccc4)cc3)n2)cc(-c2nc(-c3ccccc3)nc(-c3ccccc3)n2)c1F]

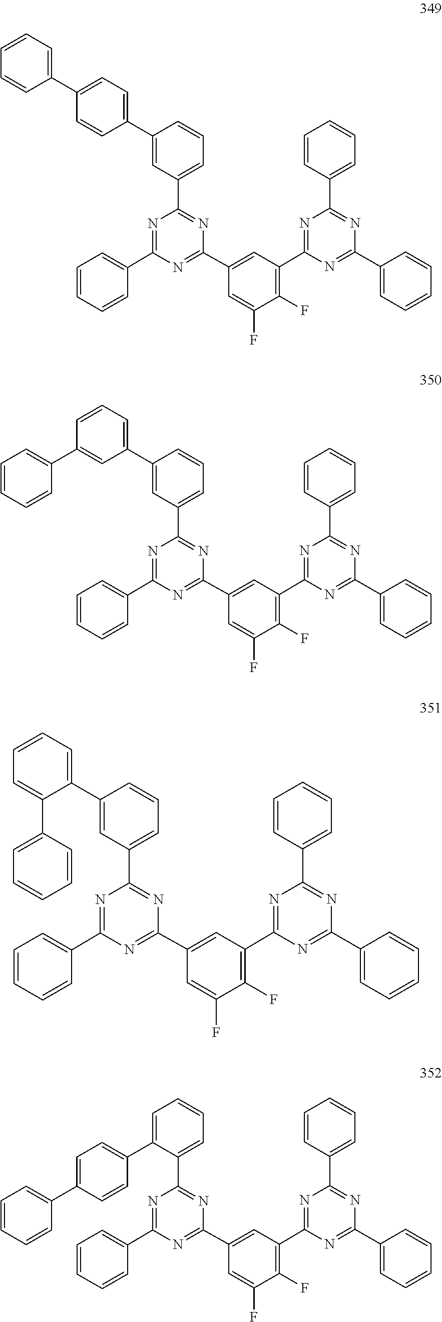 [SMILES: Fc1cc(-c2nc(-c3ccccc3)nc(-c3cccc(-c4ccc(-c5ccccc5)cc4)c3)n2)cc(-c2nc(-c3ccccc3)nc(-c3ccccc3)n2)c1F ; Fc1cc(-c2nc(-c3ccccc3)nc(-c3cccc(-c4cccc(-c5ccccc5)c4)c3)n2)cc(-c2nc(-c3ccccc3)nc(-c3ccccc3)n2)c1F ; Fc1cc(-c2nc(-c3ccccc3)nc(-c3cccc(-c4ccccc4-c4ccccc4)c3)n2)cc(-c2nc(-c3ccccc3)nc(-c3ccccc3)n2)c1F ; Fc1cc(-c2nc(-c3ccccc3)nc(-c3ccccc3-c3ccc(-c4ccccc4)cc3)n2)cc(-c2nc(-c3ccccc3)nc(-c3ccccc3)n2)c1F]

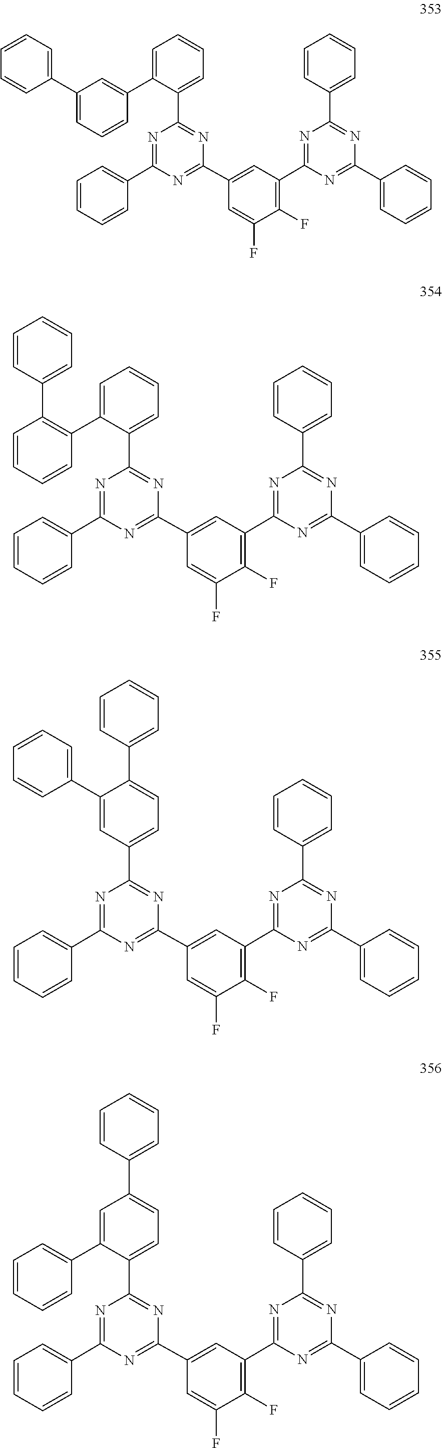 [SMILES: Fc1cc(-c2nc(-c3ccccc3)nc(-c3ccccc3-c3cccc(-c4ccccc4)c3)n2)cc(-c2nc(-c3ccccc3)nc(-c3ccccc3)n2)c1F ; Fc1cc(-c2nc(-c3ccccc3)nc(-c3ccccc3-c3ccccc3-c3ccccc3)n2)cc(-c2nc(-c3ccccc3)nc(-c3ccccc3)n2)c1F ; Fc1cc(-c2nc(-c3ccccc3)nc(-c3ccc(-c4ccccc4)c(-c4ccccc4)c3)n2)cc(-c2nc(-c3ccccc3)nc(-c3ccccc3)n2)c1F ; Fc1cc(-c2nc(-c3ccccc3)nc(-c3ccc(-c4ccccc4)cc3-c3ccccc3)n2)cc(-c2nc(-c3ccccc3)nc(-c3ccccc3)n2)c1F]

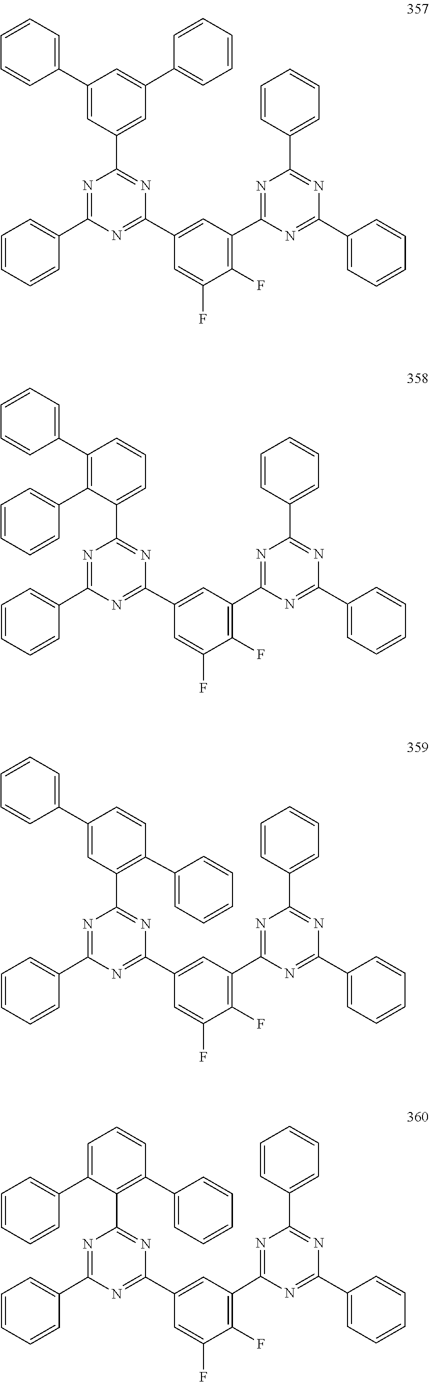 [SMILES: Fc1cc(-c2nc(-c3ccccc3)nc(-c3cc(-c4ccccc4)cc(-c4ccccc4)c3)n2)cc(-c2nc(-c3ccccc3)nc(-c3ccccc3)n2)c1F ; Fc1cc(-c2nc(-c3ccccc3)nc(-c3cccc(-c4ccccc4)c3-c3ccccc3)n2)cc(-c2nc(-c3ccccc3)nc(-c3ccccc3)n2)c1F ; Fc1cc(-c2nc(-c3ccccc3)nc(-c3cc(-c4ccccc4)ccc3-c3ccccc3)n2)cc(-c2nc(-c3ccccc3)nc(-c3ccccc3)n2)c1F ; Fc1cc(-c2nc(-c3ccccc3)nc(-c3c(-c4ccccc4)cccc3-c3ccccc3)n2)cc(-c2nc(-c3ccccc3)nc(-c3ccccc3)n2)c1F]

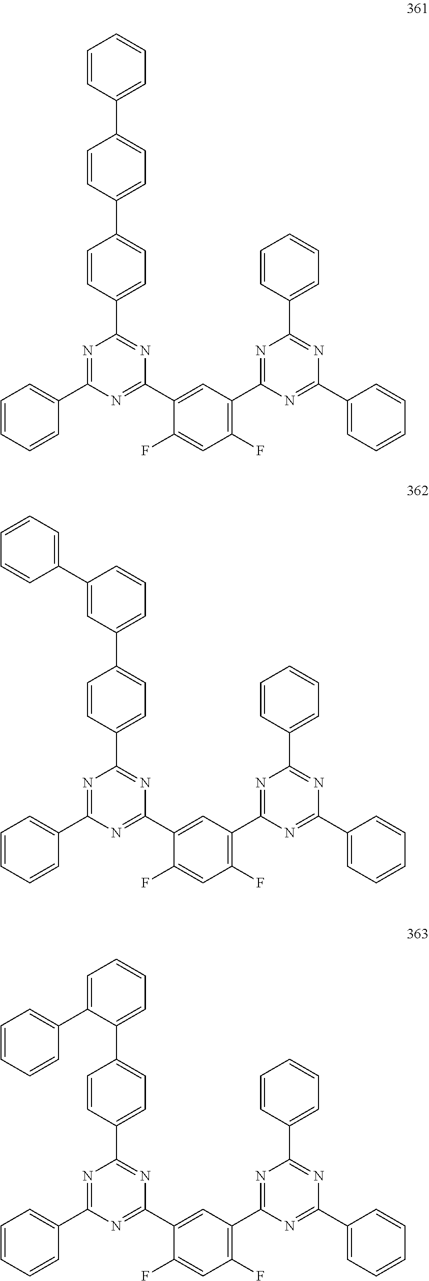 [SMILES: Fc1cc(F)c(-c2nc(-c3ccccc3)nc(-c3ccc(-c4ccc(-c5ccccc5)cc4)cc3)n2)cc1-c1nc(-c2ccccc2)nc(-c2ccccc2)n1 ; Fc1cc(F)c(-c2nc(-c3ccccc3)nc(-c3ccc(-c4cccc(-c5ccccc5)c4)cc3)n2)cc1-c1nc(-c2ccccc2)nc(-c2ccccc2)n1 ; Fc1cc(F)c(-c2nc(-c3ccccc3)nc(-c3ccc(-c4ccccc4-c4ccccc4)cc3)n2)cc1-c1nc(-c2ccccc2)nc(-c2ccccc2)n1]

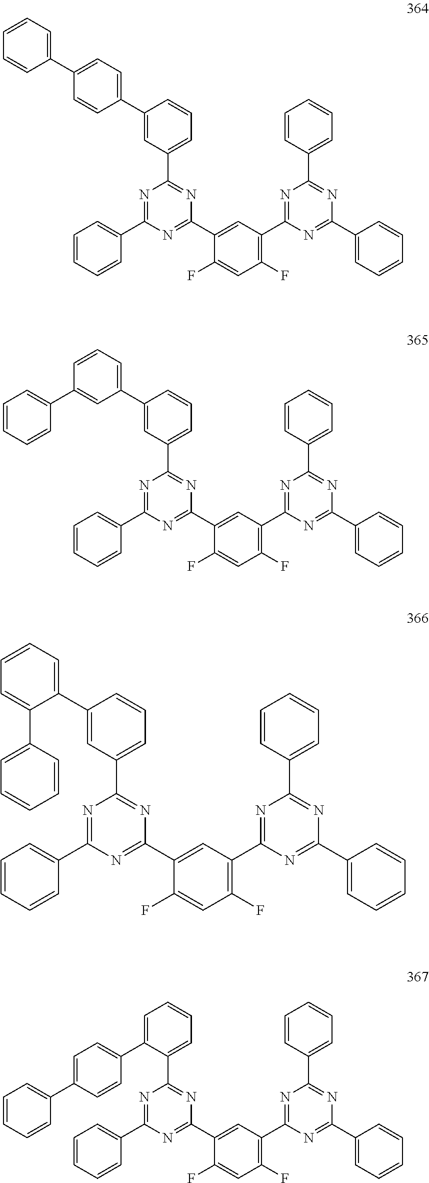 [SMILES: Fc1cc(F)c(-c2nc(-c3ccccc3)nc(-c3cccc(-c4ccc(-c5ccccc5)cc4)c3)n2)cc1-c1nc(-c2ccccc2)nc(-c2ccccc2)n1 ; Fc1cc(F)c(-c2nc(-c3ccccc3)nc(-c3cccc(-c4cccc(-c5ccccc5)c4)c3)n2)cc1-c1nc(-c2ccccc2)nc(-c2ccccc2)n1 ; Fc1cc(F)c(-c2nc(-c3ccccc3)nc(-c3cccc(-c4ccccc4-c4ccccc4)c3)n2)cc1-c1nc(-c2ccccc2)nc(-c2ccccc2)n1 ; Fc1cc(F)c(-c2nc(-c3ccccc3)nc(-c3ccccc3-c3ccc(-c4ccccc4)cc3)n2)cc1-c1nc(-c2ccccc2)nc(-c2ccccc2)n1]

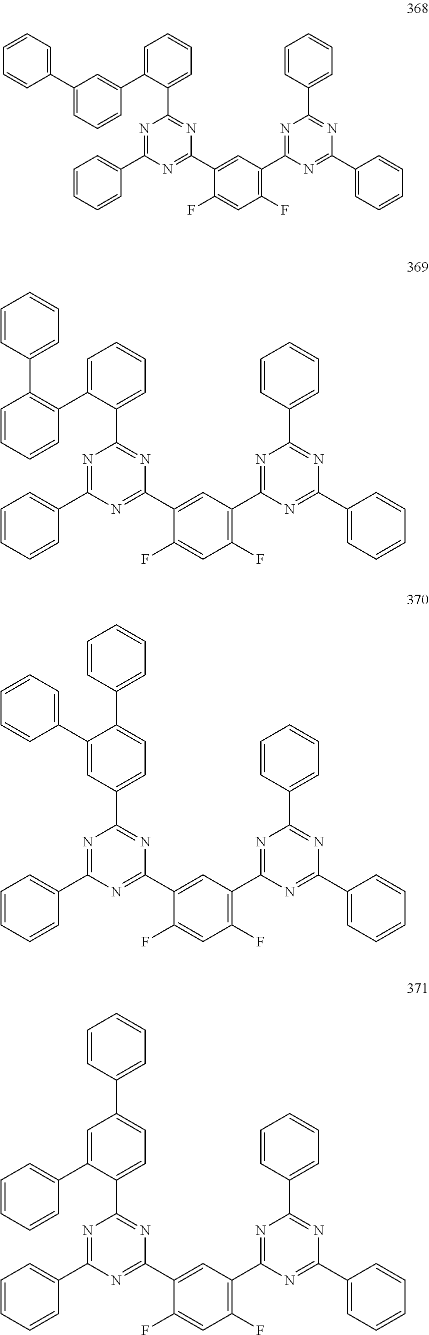 [SMILES: Fc1cc(F)c(-c2nc(-c3ccccc3)nc(-c3ccccc3-c3cccc(-c4ccccc4)c3)n2)cc1-c1nc(-c2ccccc2)nc(-c2ccccc2)n1 ; Fc1cc(F)c(-c2nc(-c3ccccc3)nc(-c3ccccc3-c3ccccc3-c3ccccc3)n2)cc1-c1nc(-c2ccccc2)nc(-c2ccccc2)n1 ; Fc1cc(F)c(-c2nc(-c3ccccc3)nc(-c3ccc(-c4ccccc4)c(-c4ccccc4)c3)n2)cc1-c1nc(-c2ccccc2)nc(-c2ccccc2)n1 ; Fc1cc(F)c(-c2nc(-c3ccccc3)nc(-c3ccc(-c4ccccc4)cc3-c3ccccc3)n2)cc1-c1nc(-c2ccccc2)nc(-c2ccccc2)n1]

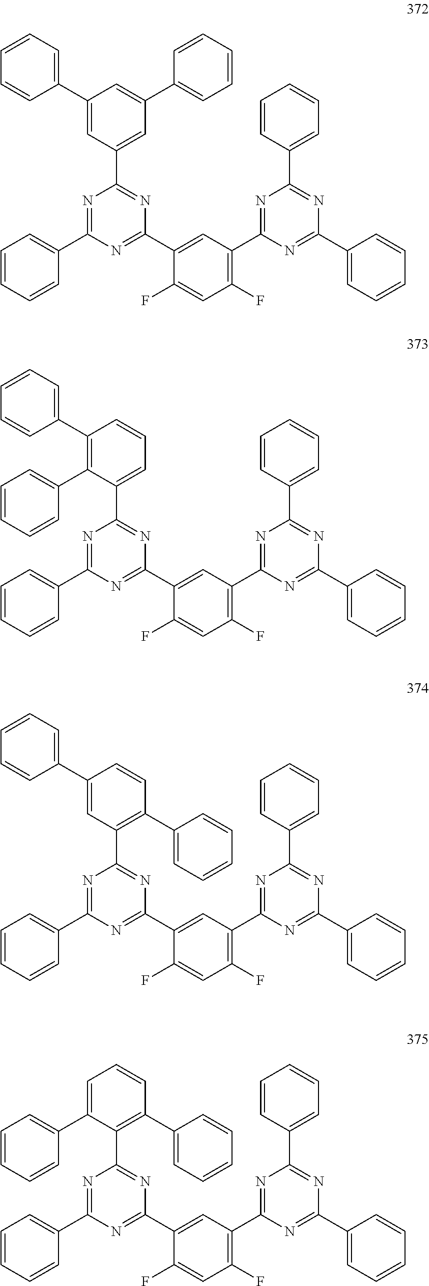 [SMILES: Fc1cc(F)c(-c2nc(-c3ccccc3)nc(-c3cc(-c4ccccc4)cc(-c4ccccc4)c3)n2)cc1-c1nc(-c2ccccc2)nc(-c2ccccc2)n1 ; Fc1cc(F)c(-c2nc(-c3ccccc3)nc(-c3cccc(-c4ccccc4)c3-c3ccccc3)n2)cc1-c1nc(-c2ccccc2)nc(-c2ccccc2)n1 ; Fc1cc(F)c(-c2nc(-c3ccccc3)nc(-c3cc(-c4ccccc4)ccc3-c3ccccc3)n2)cc1-c1nc(-c2ccccc2)nc(-c2ccccc2)n1 ; Fc1cc(F)c(-c2nc(-c3ccccc3)nc(-c3c(-c4ccccc4)cccc3-c3ccccc3)n2)cc1-c1nc(-c2ccccc2)nc(-c2ccccc2)n1]

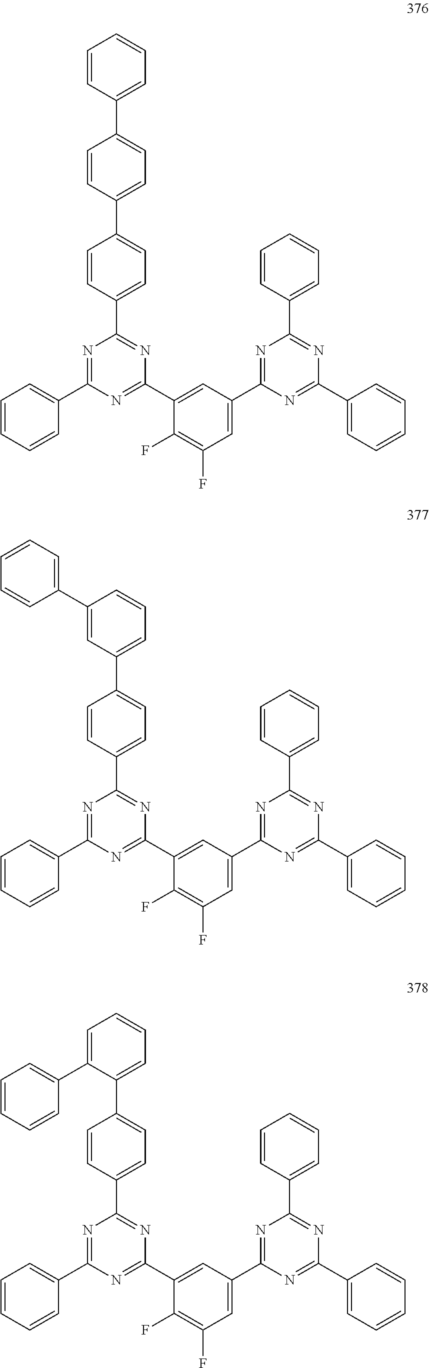 [SMILES: Fc1cc(-c2nc(-c3ccccc3)nc(-c3ccccc3)n2)cc(-c2nc(-c3ccccc3)nc(-c3ccc(-c4ccc(-c5ccccc5)cc4)cc3)n2)c1F ; Fc1cc(-c2nc(-c3ccccc3)nc(-c3ccccc3)n2)cc(-c2nc(-c3ccccc3)nc(-c3ccc(-c4cccc(-c5ccccc5)c4)cc3)n2)c1F ; Fc1cc(-c2nc(-c3ccccc3)nc(-c3ccccc3)n2)cc(-c2nc(-c3ccccc3)nc(-c3ccc(-c4ccccc4-c4ccccc4)cc3)n2)c1F]

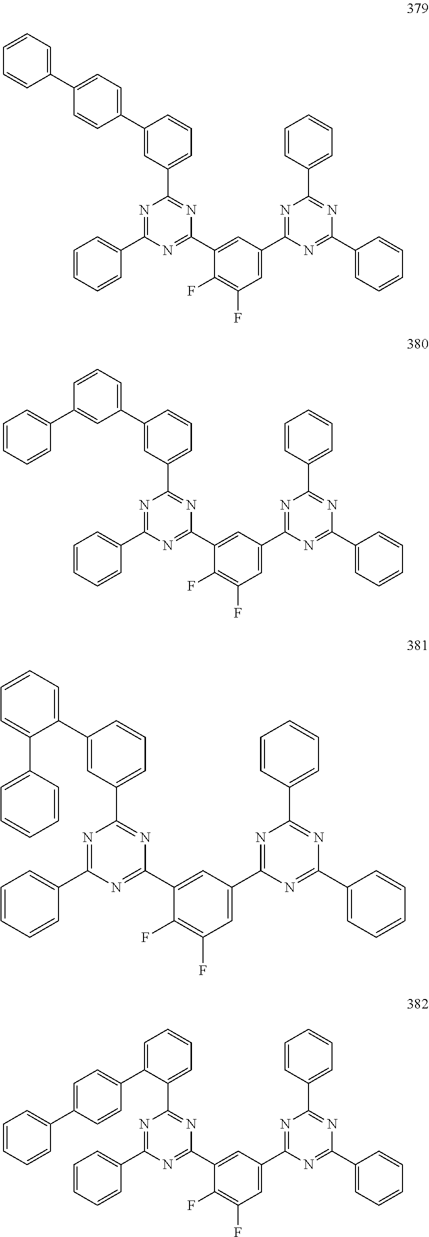 [SMILES: Fc1cc(-c2nc(-c3ccccc3)nc(-c3ccccc3)n2)cc(-c2nc(-c3ccccc3)nc(-c3cccc(-c4ccc(-c5ccccc5)cc4)c3)n2)c1F ; Fc1cc(-c2nc(-c3ccccc3)nc(-c3ccccc3)n2)cc(-c2nc(-c3ccccc3)nc(-c3cccc(-c4cccc(-c5ccccc5)c4)c3)n2)c1F ; Fc1cc(-c2nc(-c3ccccc3)nc(-c3ccccc3)n2)cc(-c2nc(-c3ccccc3)nc(-c3cccc(-c4ccccc4-c4ccccc4)c3)n2)c1F ; Fc1cc(-c2nc(-c3ccccc3)nc(-c3ccccc3)n2)cc(-c2nc(-c3ccccc3)nc(-c3ccccc3-c3ccc(-c4ccccc4)cc3)n2)c1F]

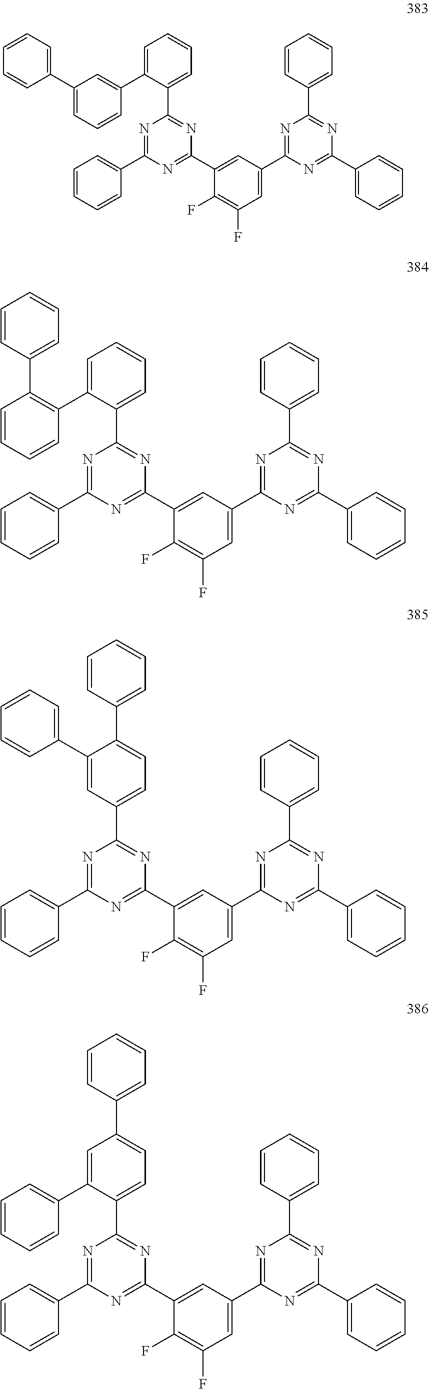 [SMILES: Fc1cc(-c2nc(-c3ccccc3)nc(-c3ccccc3)n2)cc(-c2nc(-c3ccccc3)nc(-c3ccccc3-c3cccc(-c4ccccc4)c3)n2)c1F ; Fc1cc(-c2nc(-c3ccccc3)nc(-c3ccccc3)n2)cc(-c2nc(-c3ccccc3)nc(-c3ccccc3-c3ccccc3-c3ccccc3)n2)c1F ; Fc1cc(-c2nc(-c3ccccc3)nc(-c3ccccc3)n2)cc(-c2nc(-c3ccccc3)nc(-c3ccc(-c4ccccc4)c(-c4ccccc4)c3)n2)c1F ; Fc1cc(-c2nc(-c3ccccc3)nc(-c3ccccc3)n2)cc(-c2nc(-c3ccccc3)nc(-c3ccc(-c4ccccc4)cc3-c3ccccc3)n2)c1F]

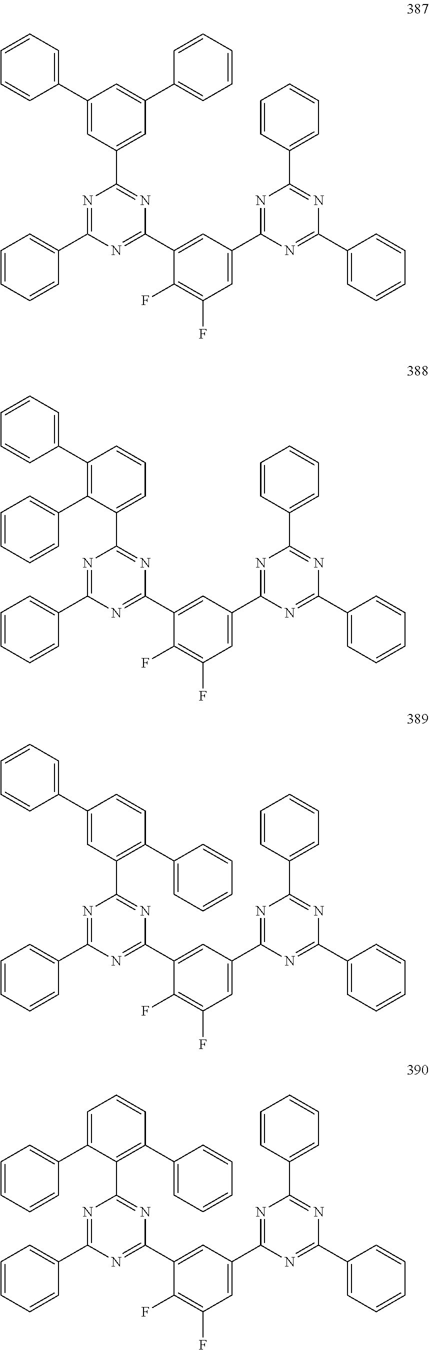 [SMILES: Fc1cc(-c2nc(-c3ccccc3)nc(-c3ccccc3)n2)cc(-c2nc(-c3ccccc3)nc(-c3cc(-c4ccccc4)cc(-c4ccccc4)c3)n2)c1F ; Fc1cc(-c2nc(-c3ccccc3)nc(-c3ccccc3)n2)cc(-c2nc(-c3ccccc3)nc(-c3cccc(-c4ccccc4)c3-c3ccccc3)n2)c1F ; Fc1cc(-c2nc(-c3ccccc3)nc(-c3ccccc3)n2)cc(-c2nc(-c3ccccc3)nc(-c3cc(-c4ccccc4)ccc3-c3ccccc3)n2)c1F ; Fc1cc(-c2nc(-c3ccccc3)nc(-c3ccccc3)n2)cc(-c2nc(-c3ccccc3)nc(-c3c(-c4ccccc4)cccc3-c3ccccc3)n2)c1F]

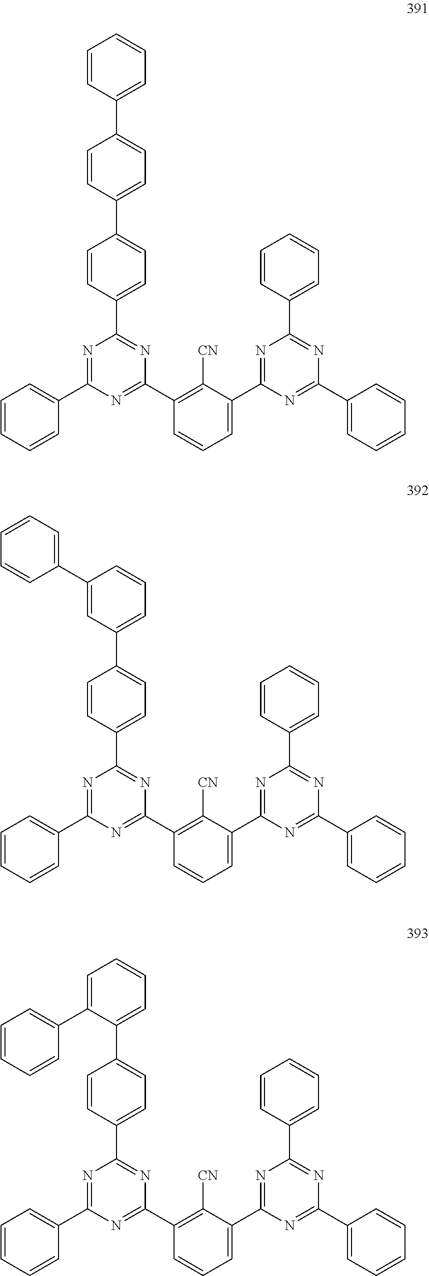 [SMILES: N#Cc1c(-c2nc(-c3ccccc3)nc(-c3ccccc3)n2)cccc1-c1nc(-c2ccccc2)nc(-c2ccc(-c3ccc(-c4ccccc4)cc3)cc2)n1 ; N#Cc1c(-c2nc(-c3ccccc3)nc(-c3ccccc3)n2)cccc1-c1nc(-c2ccccc2)nc(-c2ccc(-c3cccc(-c4ccccc4)c3)cc2)n1 ; N#Cc1c(-c2nc(-c3ccccc3)nc(-c3ccccc3)n2)cccc1-c1nc(-c2ccccc2)nc(-c2ccc(-c3ccccc3-c3ccccc3)cc2)n1]

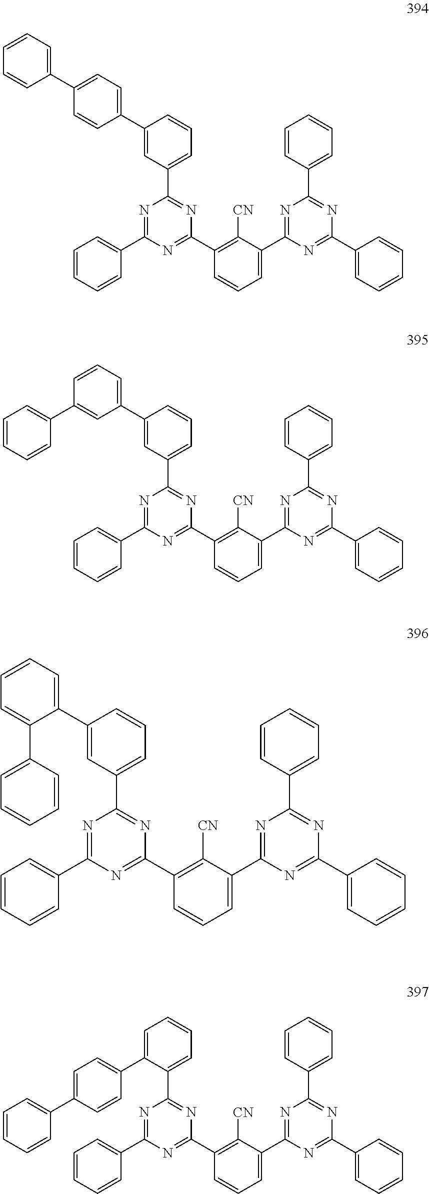 [SMILES: N#Cc1c(-c2nc(-c3ccccc3)nc(-c3ccccc3)n2)cccc1-c1nc(-c2ccccc2)nc(-c2cccc(-c3ccc(-c4ccccc4)cc3)c2)n1 ; N#Cc1c(-c2nc(-c3ccccc3)nc(-c3ccccc3)n2)cccc1-c1nc(-c2ccccc2)nc(-c2cccc(-c3cccc(-c4ccccc4)c3)c2)n1 ; N#Cc1c(-c2nc(-c3ccccc3)nc(-c3ccccc3)n2)cccc1-c1nc(-c2ccccc2)nc(-c2cccc(-c3ccccc3-c3ccccc3)c2)n1 ; N#Cc1c(-c2nc(-c3ccccc3)nc(-c3ccccc3)n2)cccc1-c1nc(-c2ccccc2)nc(-c2ccccc2-c2ccc(-c3ccccc3)cc2)n1]

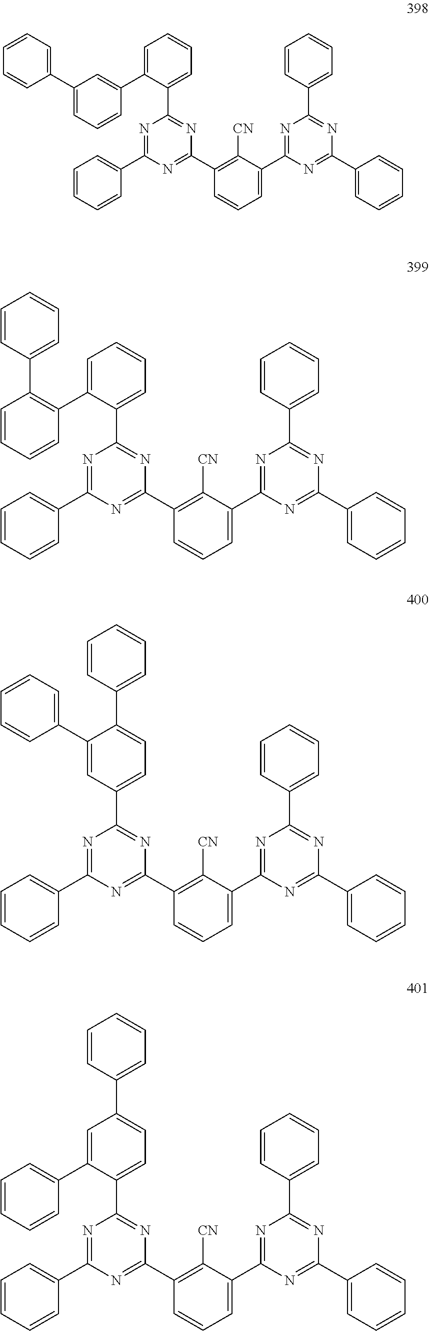 [SMILES: N#Cc1c(-c2nc(-c3ccccc3)nc(-c3ccccc3)n2)cccc1-c1nc(-c2ccccc2)nc(-c2ccccc2-c2cccc(-c3ccccc3)c2)n1 ; N#Cc1c(-c2nc(-c3ccccc3)nc(-c3ccccc3)n2)cccc1-c1nc(-c2ccccc2)nc(-c2ccccc2-c2ccccc2-c2ccccc2)n1 ; N#Cc1c(-c2nc(-c3ccccc3)nc(-c3ccccc3)n2)cccc1-c1nc(-c2ccccc2)nc(-c2ccc(-c3ccccc3)c(-c3ccccc3)c2)n1 ; N#Cc1c(-c2nc(-c3ccccc3)nc(-c3ccccc3)n2)cccc1-c1nc(-c2ccccc2)nc(-c2ccc(-c3ccccc3)cc2-c2ccccc2)n1]

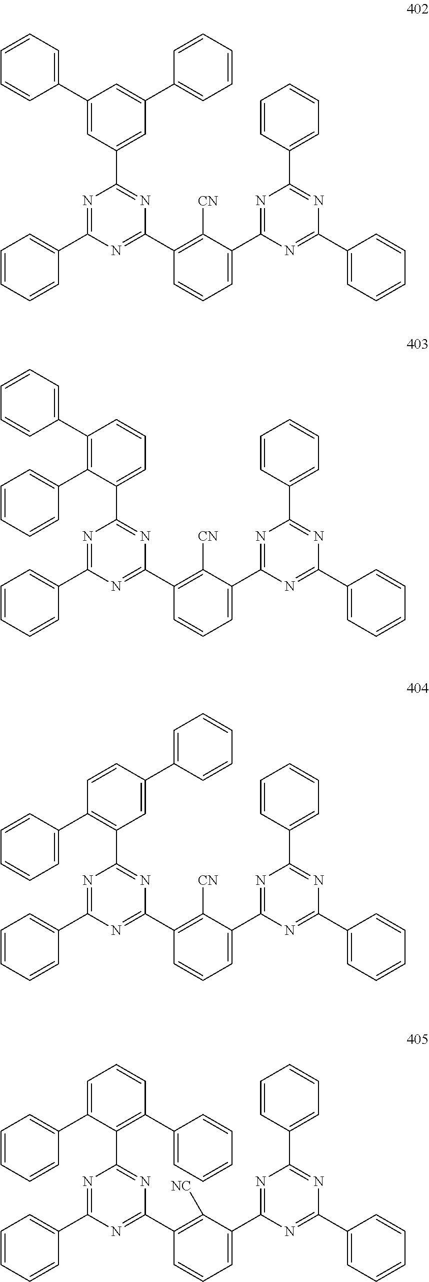 [SMILES: N#Cc1c(-c2nc(-c3ccccc3)nc(-c3ccccc3)n2)cccc1-c1nc(-c2ccccc2)nc(-c2cc(-c3ccccc3)cc(-c3ccccc3)c2)n1 ; N#Cc1c(-c2nc(-c3ccccc3)nc(-c3ccccc3)n2)cccc1-c1nc(-c2ccccc2)nc(-c2cccc(-c3ccccc3)c2-c2ccccc2)n1 ; N#Cc1c(-c2nc(-c3ccccc3)nc(-c3ccccc3)n2)cccc1-c1nc(-c2ccccc2)nc(-c2cc(-c3ccccc3)ccc2-c2ccccc2)n1 ; N#Cc1c(-c2nc(-c3ccccc3)nc(-c3ccccc3)n2)cccc1-c1nc(-c2ccccc2)nc(-c2c(-c3ccccc3)cccc2-c2ccccc2)n1]

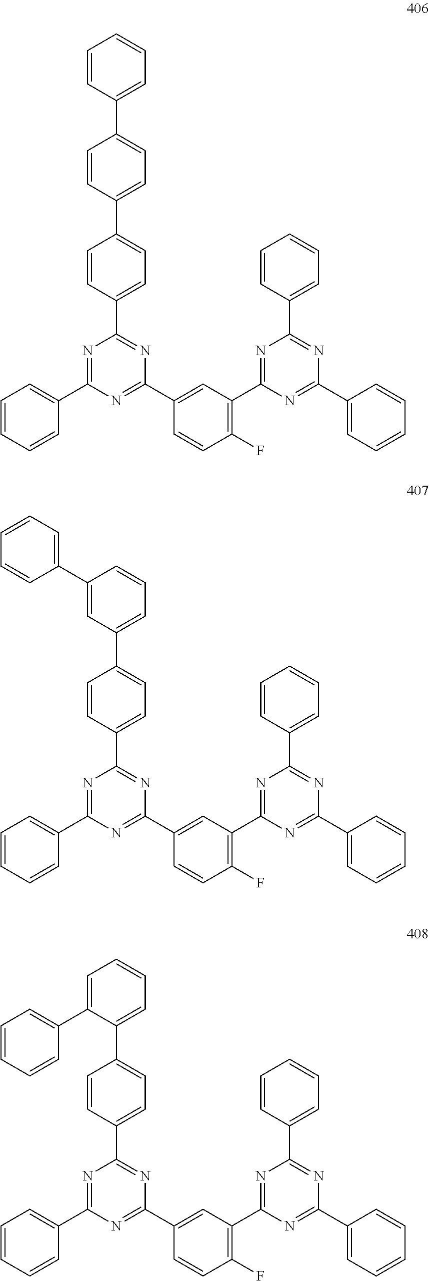 [SMILES: Fc1ccc(-c2nc(-c3ccccc3)nc(-c3ccc(-c4ccc(-c5ccccc5)cc4)cc3)n2)cc1-c1nc(-c2ccccc2)nc(-c2ccccc2)n1 ; Fc1ccc(-c2nc(-c3ccccc3)nc(-c3ccc(-c4cccc(-c5ccccc5)c4)cc3)n2)cc1-c1nc(-c2ccccc2)nc(-c2ccccc2)n1 ; Fc1ccc(-c2nc(-c3ccccc3)nc(-c3ccc(-c4ccccc4-c4ccccc4)cc3)n2)cc1-c1nc(-c2ccccc2)nc(-c2ccccc2)n1]

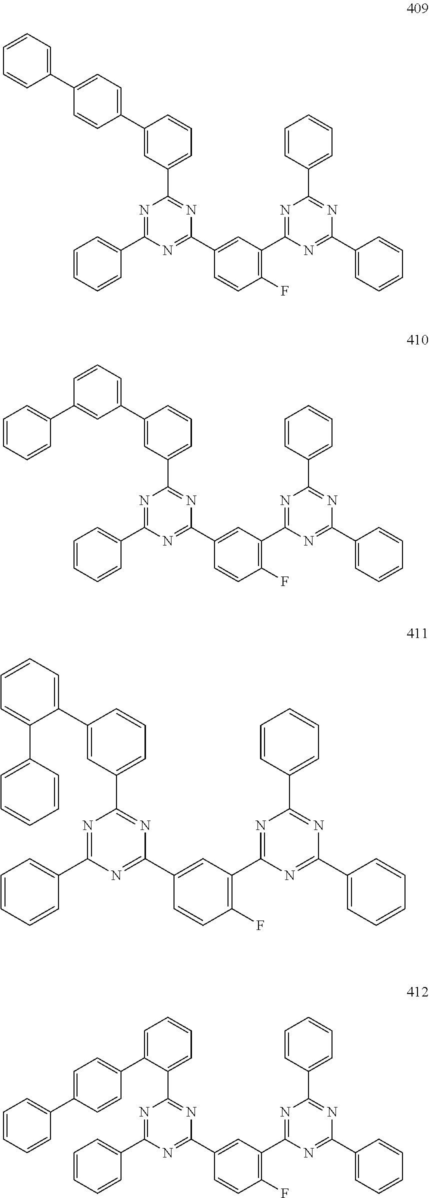 [SMILES: Fc1ccc(-c2nc(-c3ccccc3)nc(-c3cccc(-c4ccc(-c5ccccc5)cc4)c3)n2)cc1-c1nc(-c2ccccc2)nc(-c2ccccc2)n1 ; Fc1ccc(-c2nc(-c3ccccc3)nc(-c3cccc(-c4cccc(-c5ccccc5)c4)c3)n2)cc1-c1nc(-c2ccccc2)nc(-c2ccccc2)n1 ; Fc1ccc(-c2nc(-c3ccccc3)nc(-c3cccc(-c4ccccc4-c4ccccc4)c3)n2)cc1-c1nc(-c2ccccc2)nc(-c2ccccc2)n1 ; Fc1ccc(-c2nc(-c3ccccc3)nc(-c3ccccc3-c3ccc(-c4ccccc4)cc3)n2)cc1-c1nc(-c2ccccc2)nc(-c2ccccc2)n1]

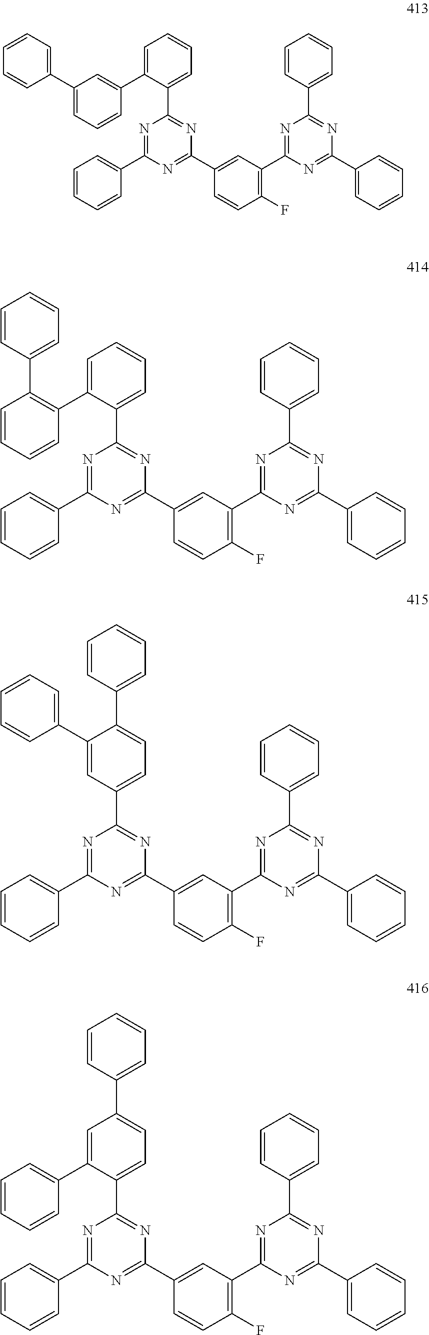 [SMILES: Fc1ccc(-c2nc(-c3ccccc3)nc(-c3ccccc3-c3cccc(-c4ccccc4)c3)n2)cc1-c1nc(-c2ccccc2)nc(-c2ccccc2)n1 ; Fc1ccc(-c2nc(-c3ccccc3)nc(-c3ccccc3-c3ccccc3-c3ccccc3)n2)cc1-c1nc(-c2ccccc2)nc(-c2ccccc2)n1 ; Fc1ccc(-c2nc(-c3ccccc3)nc(-c3ccc(-c4ccccc4)c(-c4ccccc4)c3)n2)cc1-c1nc(-c2ccccc2)nc(-c2ccccc2)n1 ; Fc1ccc(-c2nc(-c3ccccc3)nc(-c3ccc(-c4ccccc4)cc3-c3ccccc3)n2)cc1-c1nc(-c2ccccc2)nc(-c2ccccc2)n1]

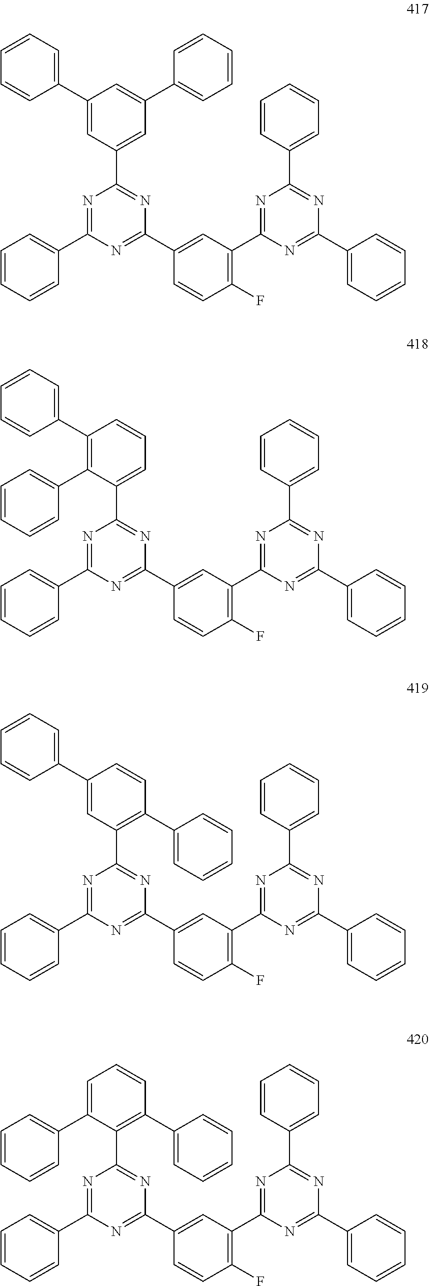 [SMILES: Fc1ccc(-c2nc(-c3ccccc3)nc(-c3cc(-c4ccccc4)cc(-c4ccccc4)c3)n2)cc1-c1nc(-c2ccccc2)nc(-c2ccccc2)n1 ; Fc1ccc(-c2nc(-c3ccccc3)nc(-c3cccc(-c4ccccc4)c3-c3ccccc3)n2)cc1-c1nc(-c2ccccc2)nc(-c2ccccc2)n1 ; Fc1ccc(-c2nc(-c3ccccc3)nc(-c3cc(-c4ccccc4)ccc3-c3ccccc3)n2)cc1-c1nc(-c2ccccc2)nc(-c2ccccc2)n1 ; Fc1ccc(-c2nc(-c3ccccc3)nc(-c3c(-c4ccccc4)cccc3-c3ccccc3)n2)cc1-c1nc(-c2ccccc2)nc(-c2ccccc2)n1]

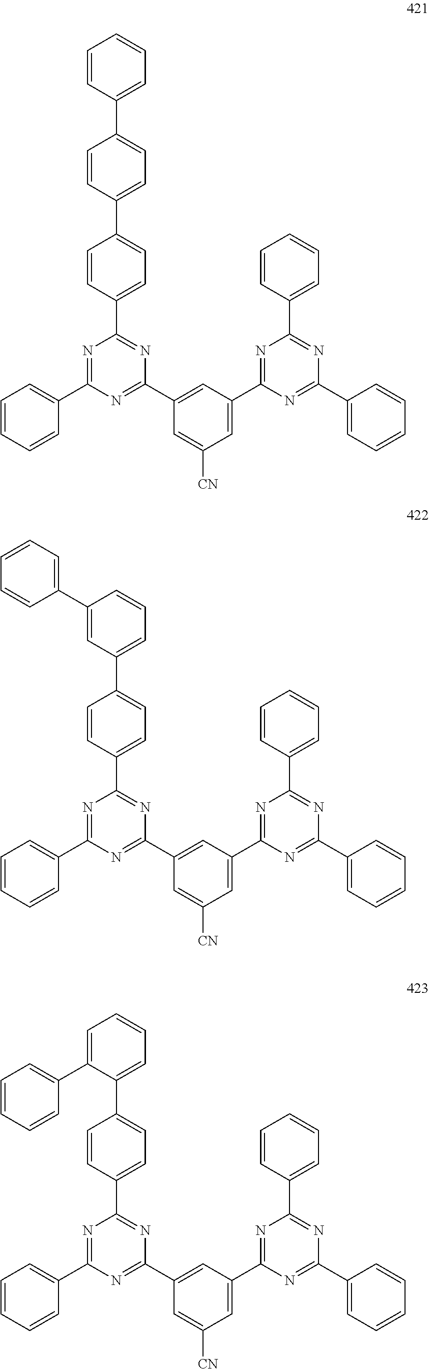 [SMILES: N#Cc1cc(-c2nc(-c3ccccc3)nc(-c3ccccc3)n2)cc(-c2nc(-c3ccccc3)nc(-c3ccc(-c4ccc(-c5ccccc5)cc4)cc3)n2)c1 ; N#Cc1cc(-c2nc(-c3ccccc3)nc(-c3ccccc3)n2)cc(-c2nc(-c3ccccc3)nc(-c3ccc(-c4cccc(-c5ccccc5)c4)cc3)n2)c1 ; N#Cc1cc(-c2nc(-c3ccccc3)nc(-c3ccccc3)n2)cc(-c2nc(-c3ccccc3)nc(-c3ccc(-c4ccccc4-c4ccccc4)cc3)n2)c1]

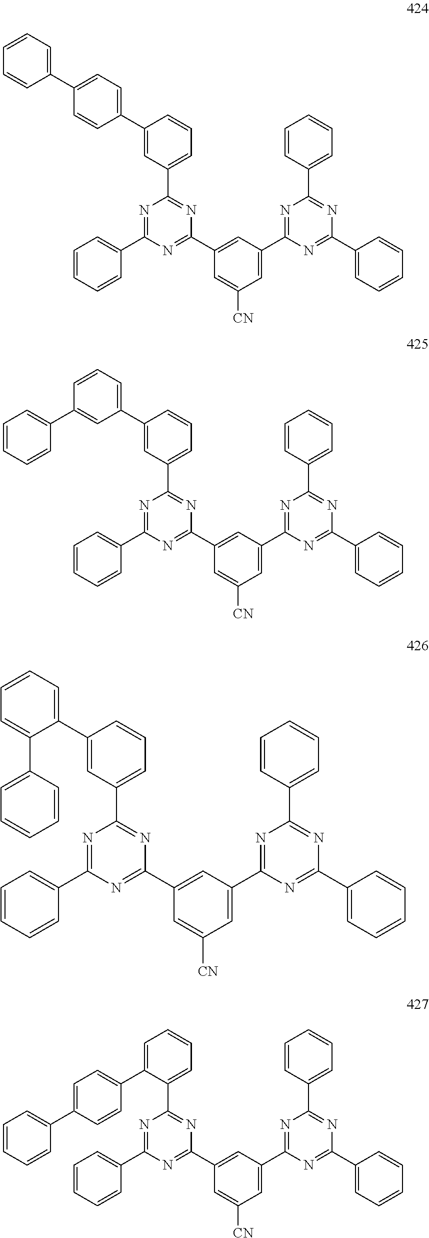 [SMILES: N#Cc1cc(-c2nc(-c3ccccc3)nc(-c3ccccc3)n2)cc(-c2nc(-c3ccccc3)nc(-c3cccc(-c4ccc(-c5ccccc5)cc4)c3)n2)c1 ; N#Cc1cc(-c2nc(-c3ccccc3)nc(-c3ccccc3)n2)cc(-c2nc(-c3ccccc3)nc(-c3cccc(-c4cccc(-c5ccccc5)c4)c3)n2)c1 ; N#Cc1cc(-c2nc(-c3ccccc3)nc(-c3ccccc3)n2)cc(-c2nc(-c3ccccc3)nc(-c3cccc(-c4ccccc4-c4ccccc4)c3)n2)c1 ; N#Cc1cc(-c2nc(-c3ccccc3)nc(-c3ccccc3)n2)cc(-c2nc(-c3ccccc3)nc(-c3ccccc3-c3ccc(-c4ccccc4)cc3)n2)c1]

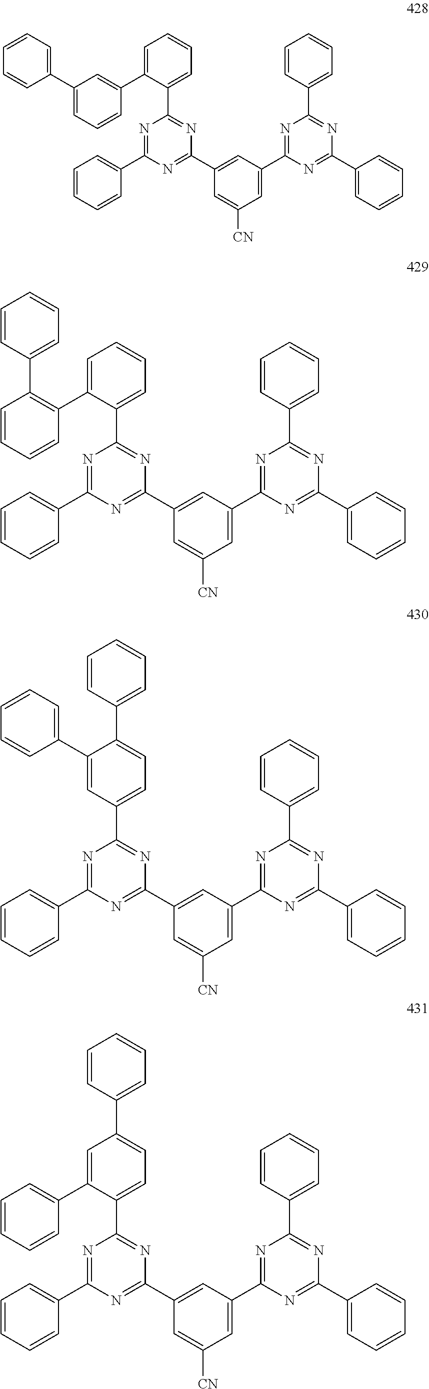 [SMILES: N#Cc1cc(-c2nc(-c3ccccc3)nc(-c3ccccc3)n2)cc(-c2nc(-c3ccccc3)nc(-c3ccccc3-c3cccc(-c4ccccc4)c3)n2)c1 ; N#Cc1cc(-c2nc(-c3ccccc3)nc(-c3ccccc3)n2)cc(-c2nc(-c3ccccc3)nc(-c3ccccc3-c3ccccc3-c3ccccc3)n2)c1 ; N#Cc1cc(-c2nc(-c3ccccc3)nc(-c3ccccc3)n2)cc(-c2nc(-c3ccccc3)nc(-c3ccc(-c4ccccc4)c(-c4ccccc4)c3)n2)c1 ; N#Cc1cc(-c2nc(-c3ccccc3)nc(-c3ccccc3)n2)cc(-c2nc(-c3ccccc3)nc(-c3ccc(-c4ccccc4)cc3-c3ccccc3)n2)c1]

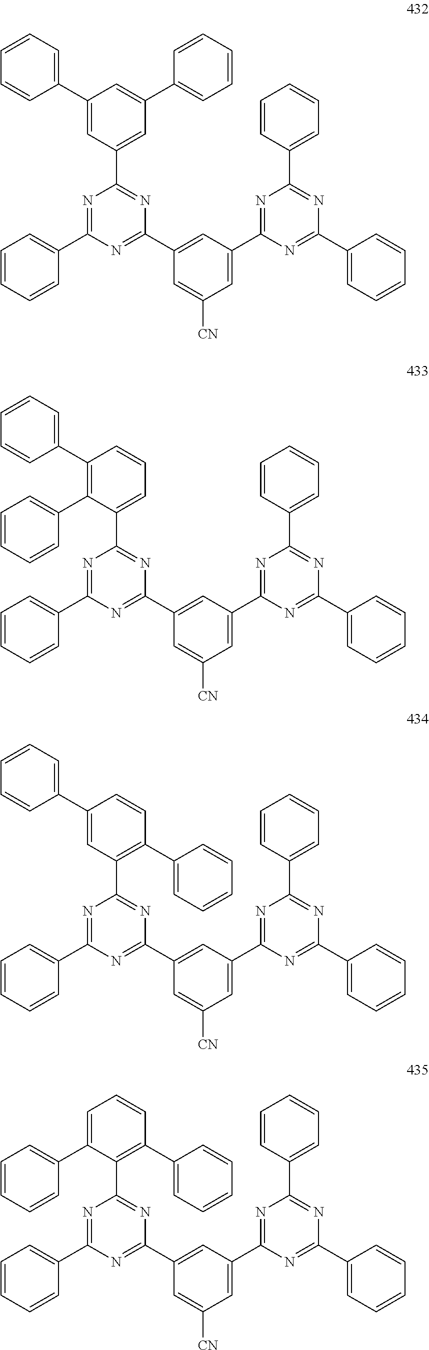 [SMILES: N#Cc1cc(-c2nc(-c3ccccc3)nc(-c3ccccc3)n2)cc(-c2nc(-c3ccccc3)nc(-c3cc(-c4ccccc4)cc(-c4ccccc4)c3)n2)c1 ; N#Cc1cc(-c2nc(-c3ccccc3)nc(-c3ccccc3)n2)cc(-c2nc(-c3ccccc3)nc(-c3cccc(-c4ccccc4)c3-c3ccccc3)n2)c1 ; N#Cc1cc(-c2nc(-c3ccccc3)nc(-c3ccccc3)n2)cc(-c2nc(-c3ccccc3)nc(-c3cc(-c4ccccc4)ccc3-c3ccccc3)n2)c1 ; N#Cc1cc(-c2nc(-c3ccccc3)nc(-c3ccccc3)n2)cc(-c2nc(-c3ccccc3)nc(-c3c(-c4ccccc4)cccc3-c3ccccc3)n2)c1]

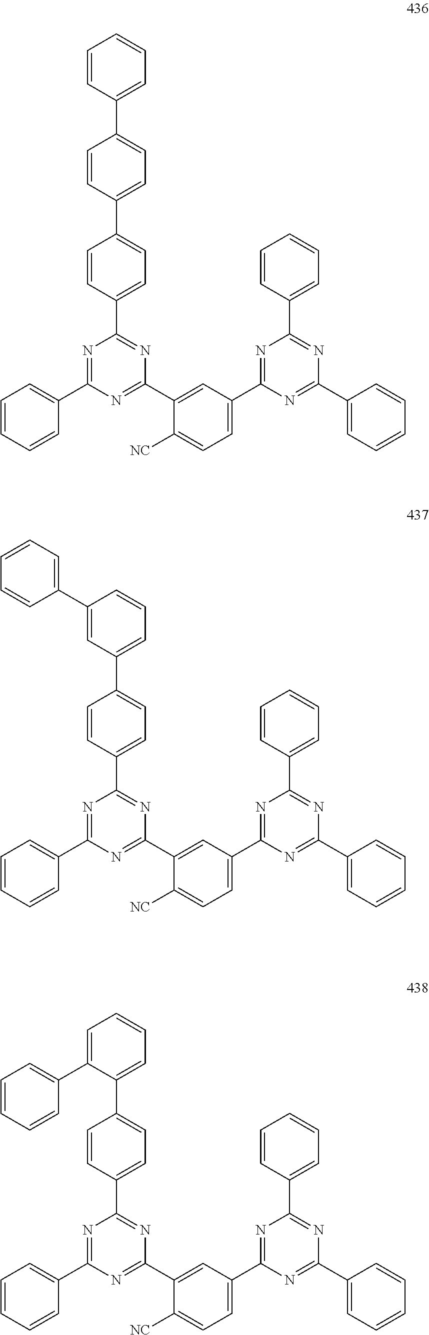 [SMILES: N#Cc1ccc(-c2nc(-c3ccccc3)nc(-c3ccccc3)n2)cc1-c1nc(-c2ccccc2)nc(-c2ccc(-c3ccc(-c4ccccc4)cc3)cc2)n1 ; N#Cc1ccc(-c2nc(-c3ccccc3)nc(-c3ccccc3)n2)cc1-c1nc(-c2ccccc2)nc(-c2ccc(-c3cccc(-c4ccccc4)c3)cc2)n1 ; N#Cc1ccc(-c2nc(-c3ccccc3)nc(-c3ccccc3)n2)cc1-c1nc(-c2ccccc2)nc(-c2ccc(-c3ccccc3-c3ccccc3)cc2)n1]

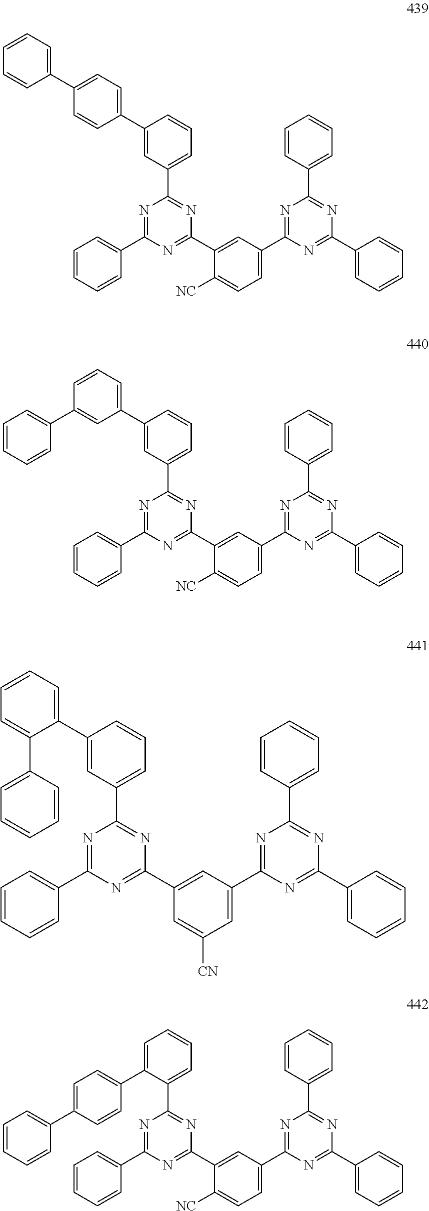 [SMILES: N#Cc1ccc(-c2nc(-c3ccccc3)nc(-c3ccccc3)n2)cc1-c1nc(-c2ccccc2)nc(-c2cccc(-c3ccc(-c4ccccc4)cc3)c2)n1 ; N#Cc1ccc(-c2nc(-c3ccccc3)nc(-c3ccccc3)n2)cc1-c1nc(-c2ccccc2)nc(-c2cccc(-c3cccc(-c4ccccc4)c3)c2)n1 ; N#Cc1cc(-c2nc(-c3ccccc3)nc(-c3ccccc3)n2)cc(-c2nc(-c3ccccc3)nc(-c3cccc(-c4ccccc4-c4ccccc4)c3)n2)c1 ; N#Cc1ccc(-c2nc(-c3ccccc3)nc(-c3ccccc3)n2)cc1-c1nc(-c2ccccc2)nc(-c2ccccc2-c2ccc(-c3ccccc3)cc2)n1]

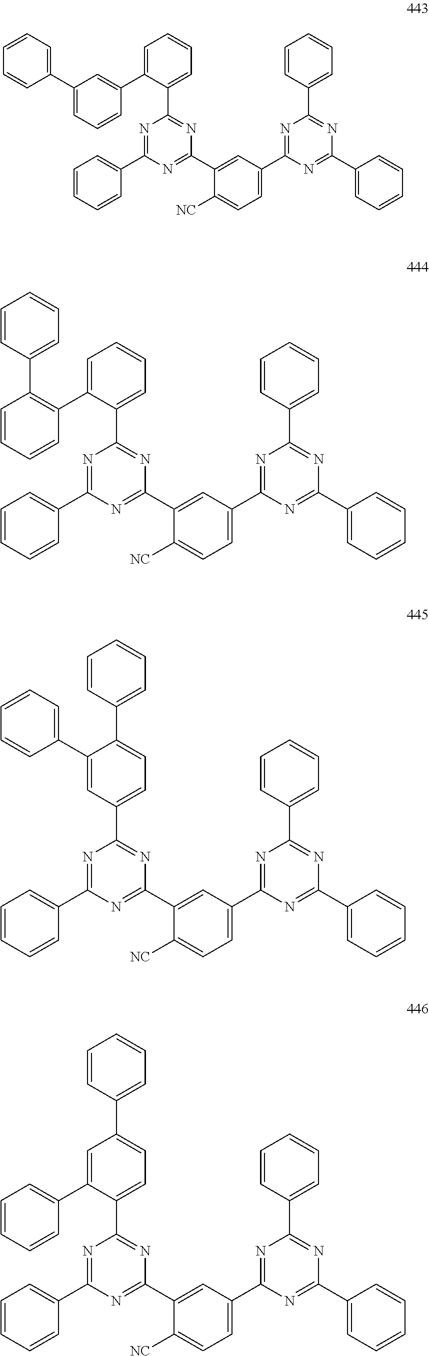 [SMILES: N#Cc1ccc(-c2nc(-c3ccccc3)nc(-c3ccccc3)n2)cc1-c1nc(-c2ccccc2)nc(-c2ccccc2-c2cccc(-c3ccccc3)c2)n1 ; N#Cc1ccc(-c2nc(-c3ccccc3)nc(-c3ccccc3)n2)cc1-c1nc(-c2ccccc2)nc(-c2ccccc2-c2ccccc2-c2ccccc2)n1 ; N#Cc1ccc(-c2nc(-c3ccccc3)nc(-c3ccccc3)n2)cc1-c1nc(-c2ccccc2)nc(-c2ccc(-c3ccccc3)c(-c3ccccc3)c2)n1 ; N#Cc1ccc(-c2nc(-c3ccccc3)nc(-c3ccccc3)n2)cc1-c1nc(-c2ccccc2)nc(-c2ccc(-c3ccccc3)cc2-c2ccccc2)n1]

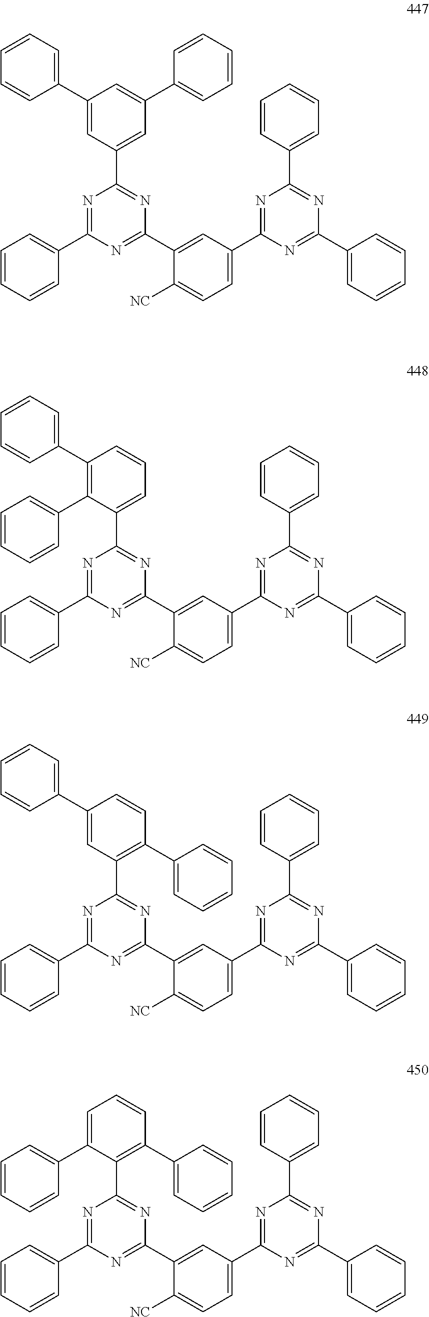 [SMILES: N#Cc1ccc(-c2nc(-c3ccccc3)nc(-c3ccccc3)n2)cc1-c1nc(-c2ccccc2)nc(-c2cc(-c3ccccc3)cc(-c3ccccc3)c2)n1 ; N#Cc1ccc(-c2nc(-c3ccccc3)nc(-c3ccccc3)n2)cc1-c1nc(-c2ccccc2)nc(-c2cccc(-c3ccccc3)c2-c2ccccc2)n1 ; N#Cc1ccc(-c2nc(-c3ccccc3)nc(-c3ccccc3)n2)cc1-c1nc(-c2ccccc2)nc(-c2cc(-c3ccccc3)ccc2-c2ccccc2)n1 ; N#Cc1ccc(-c2nc(-c3ccccc3)nc(-c3ccccc3)n2)cc1-c1nc(-c2ccccc2)nc(-c2c(-c3ccccc3)cccc2-c2ccccc2)n1]

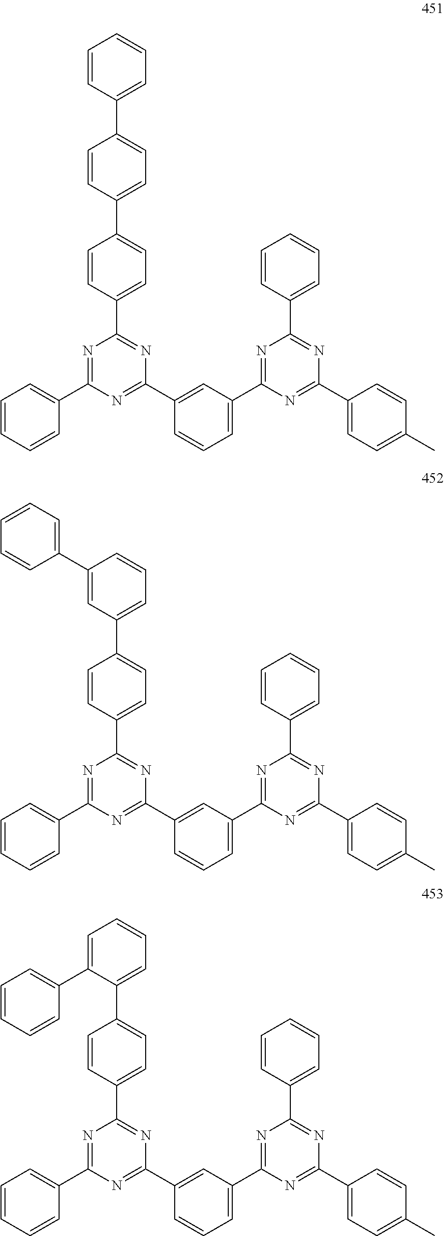 [SMILES: Cc1ccc(-c2nc(-c3ccccc3)nc(-c3cccc(-c4nc(-c5ccccc5)nc(-c5ccc(-c6ccc(-c7ccccc7)cc6)cc5)n4)c3)n2)cc1 ; Cc1ccc(-c2nc(-c3ccccc3)nc(-c3cccc(-c4nc(-c5ccccc5)nc(-c5ccc(-c6cccc(-c7ccccc7)c6)cc5)n4)c3)n2)cc1 ; Cc1ccc(-c2nc(-c3ccccc3)nc(-c3cccc(-c4nc(-c5ccccc5)nc(-c5ccc(-c6ccccc6-c6ccccc6)cc5)n4)c3)n2)cc1]

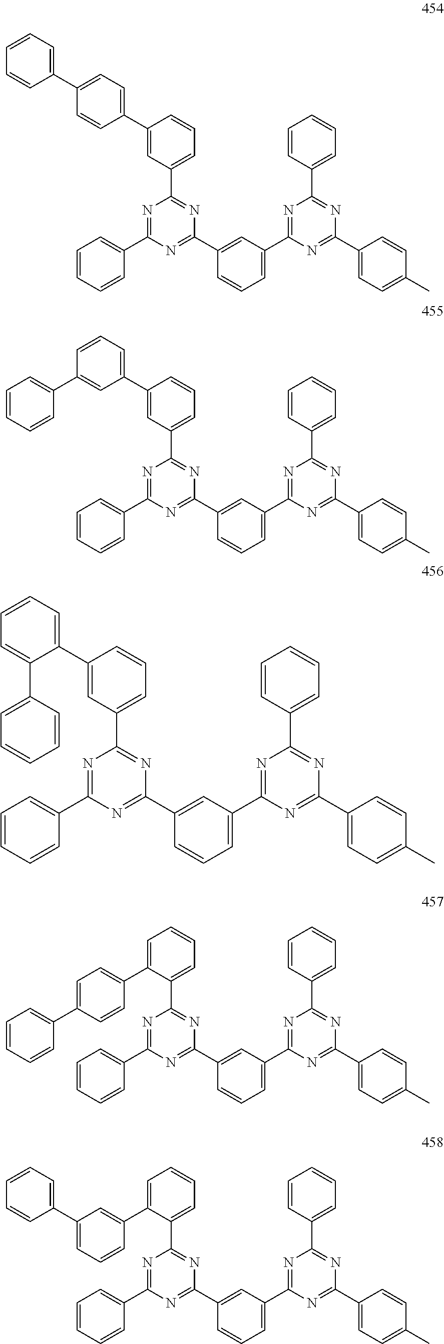 [SMILES: Cc1ccc(-c2nc(-c3ccccc3)nc(-c3cccc(-c4nc(-c5ccccc5)nc(-c5cccc(-c6ccc(-c7ccccc7)cc6)c5)n4)c3)n2)cc1 ; Cc1ccc(-c2nc(-c3ccccc3)nc(-c3cccc(-c4nc(-c5ccccc5)nc(-c5cccc(-c6cccc(-c7ccccc7)c6)c5)n4)c3)n2)cc1 ; Cc1ccc(-c2nc(-c3ccccc3)nc(-c3cccc(-c4nc(-c5ccccc5)nc(-c5cccc(-c6ccccc6-c6ccccc6)c5)n4)c3)n2)cc1 ; Cc1ccc(-c2nc(-c3ccccc3)nc(-c3cccc(-c4nc(-c5ccccc5)nc(-c5ccccc5-c5ccc(-c6ccccc6)cc5)n4)c3)n2)cc1 ; Cc1ccc(-c2nc(-c3ccccc3)nc(-c3cccc(-c4nc(-c5ccccc5)nc(-c5ccccc5-c5cccc(-c6ccccc6)c5)n4)c3)n2)cc1]

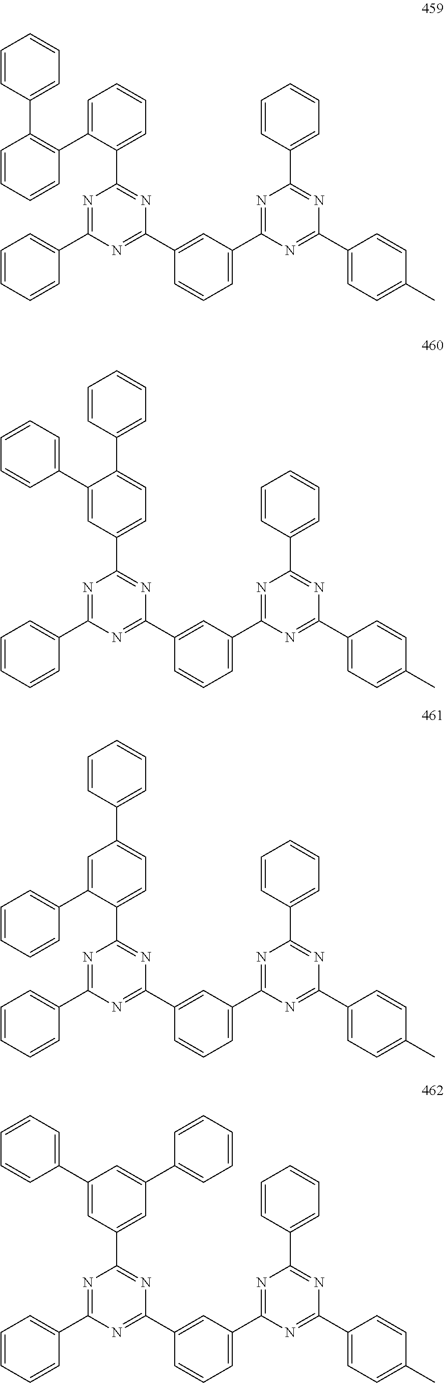 [SMILES: Cc1ccc(-c2nc(-c3ccccc3)nc(-c3cccc(-c4nc(-c5ccccc5)nc(-c5ccccc5-c5ccccc5-c5ccccc5)n4)c3)n2)cc1 ; Cc1ccc(-c2nc(-c3ccccc3)nc(-c3cccc(-c4nc(-c5ccccc5)nc(-c5ccc(-c6ccccc6)c(-c6ccccc6)c5)n4)c3)n2)cc1 ; Cc1ccc(-c2nc(-c3ccccc3)nc(-c3cccc(-c4nc(-c5ccccc5)nc(-c5ccc(-c6ccccc6)cc5-c5ccccc5)n4)c3)n2)cc1 ; Cc1ccc(-c2nc(-c3ccccc3)nc(-c3cccc(-c4nc(-c5ccccc5)nc(-c5cc(-c6ccccc6)cc(-c6ccccc6)c5)n4)c3)n2)cc1]

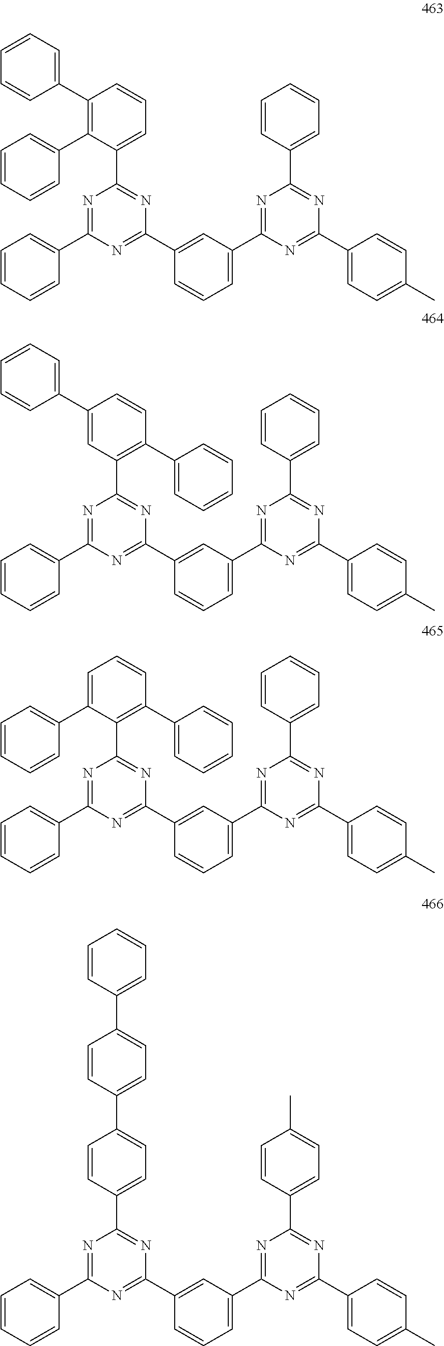

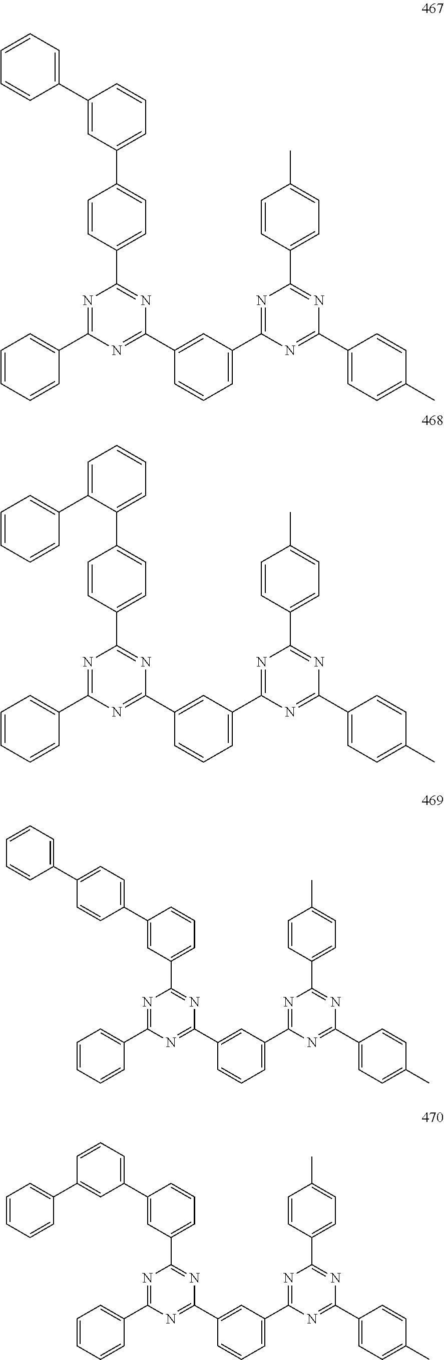 [SMILES: Cc1ccc(-c2nc(-c3ccc(C)cc3)nc(-c3cccc(-c4nc(-c5ccccc5)nc(-c5ccc(-c6cccc(-c7ccccc7)c6)cc5)n4)c3)n2)cc1 ; Cc1ccc(-c2nc(-c3ccc(C)cc3)nc(-c3cccc(-c4nc(-c5ccccc5)nc(-c5ccc(-c6ccccc6-c6ccccc6)cc5)n4)c3)n2)cc1 ; Cc1ccc(-c2nc(-c3ccc(C)cc3)nc(-c3cccc(-c4nc(-c5ccccc5)nc(-c5cccc(-c6ccc(-c7ccccc7)cc6)c5)n4)c3)n2)cc1 ; Cc1ccc(-c2nc(-c3ccc(C)cc3)nc(-c3cccc(-c4nc(-c5ccccc5)nc(-c5cccc(-c6cccc(-c7ccccc7)c6)c5)n4)c3)n2)cc1]

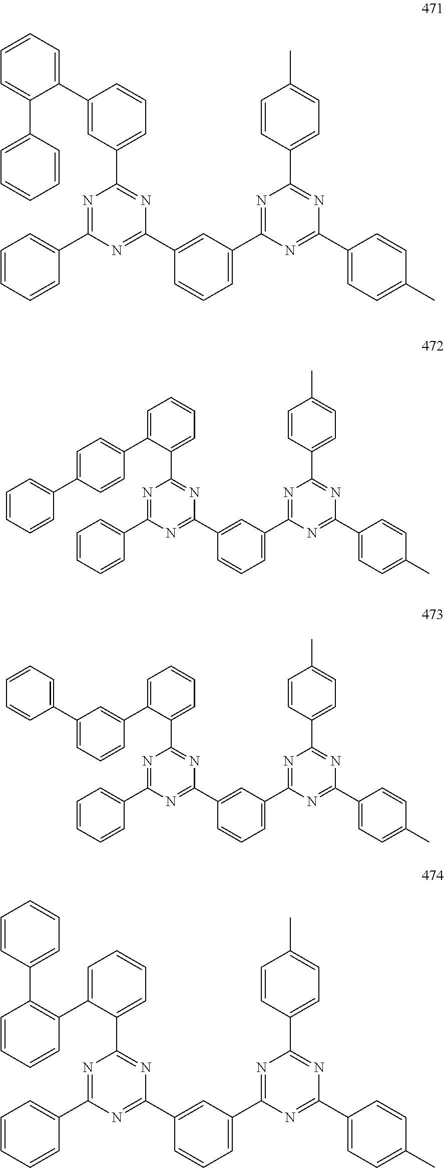 [SMILES: Cc1ccc(-c2nc(-c3ccc(C)cc3)nc(-c3cccc(-c4nc(-c5ccccc5)nc(-c5cccc(-c6ccccc6-c6ccccc6)c5)n4)c3)n2)cc1 ; Cc1ccc(-c2nc(-c3ccc(C)cc3)nc(-c3cccc(-c4nc(-c5ccccc5)nc(-c5ccccc5-c5ccc(-c6ccccc6)cc5)n4)c3)n2)cc1 ; Cc1ccc(-c2nc(-c3ccc(C)cc3)nc(-c3cccc(-c4nc(-c5ccccc5)nc(-c5ccccc5-c5cccc(-c6ccccc6)c5)n4)c3)n2)cc1 ; Cc1ccc(-c2nc(-c3ccc(C)cc3)nc(-c3cccc(-c4nc(-c5ccccc5)nc(-c5ccccc5-c5ccccc5-c5ccccc5)n4)c3)n2)cc1]

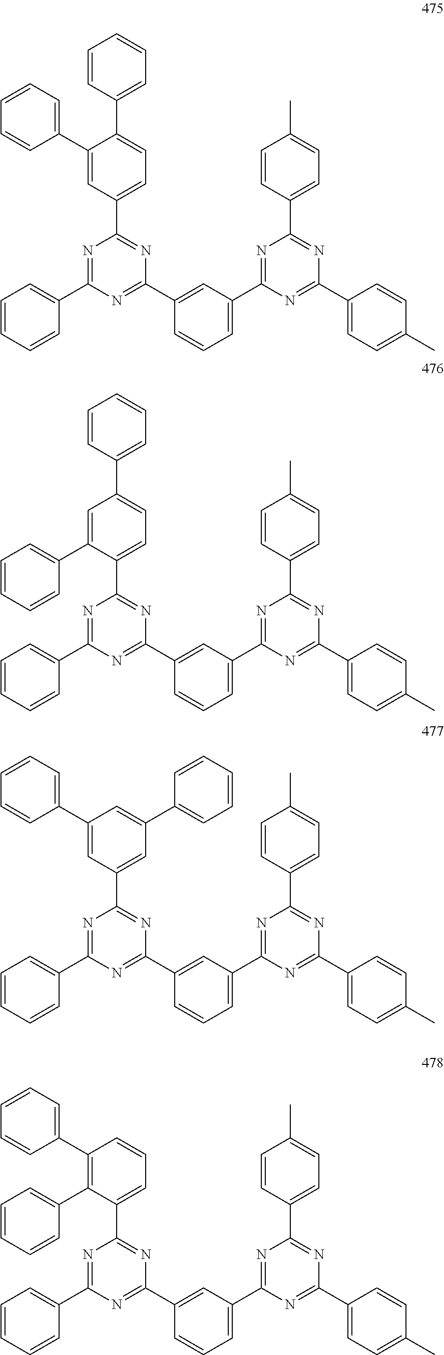

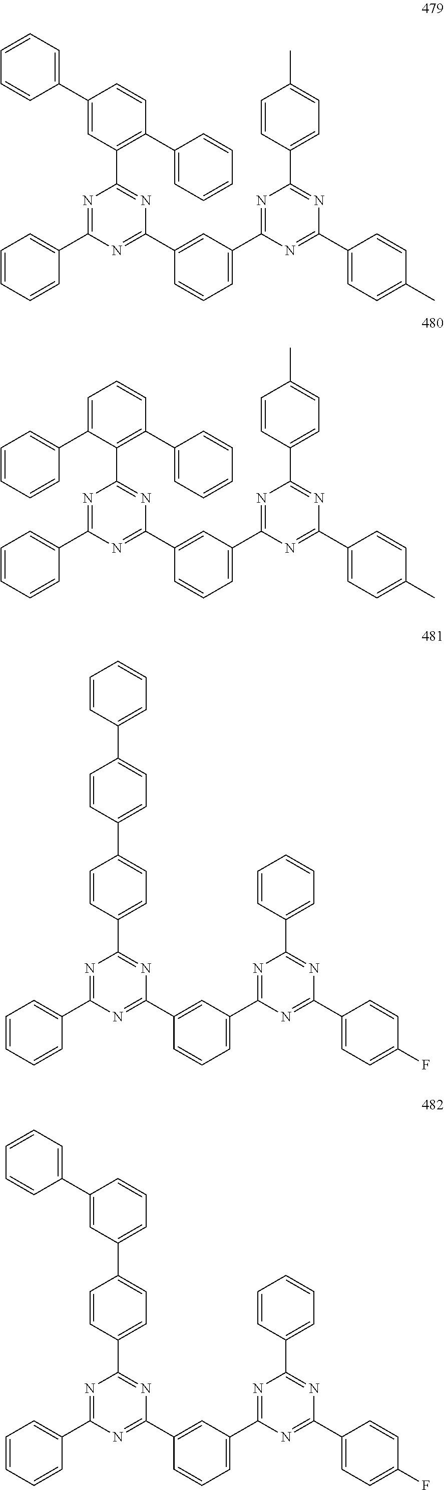 [SMILES: Cc1ccc(-c2nc(-c3ccc(C)cc3)nc(-c3cccc(-c4nc(-c5ccccc5)nc(-c5cc(-c6ccccc6)ccc5-c5ccccc5)n4)c3)n2)cc1 ; Cc1ccc(-c2nc(-c3ccc(C)cc3)nc(-c3cccc(-c4nc(-c5ccccc5)nc(-c5c(-c6ccccc6)cccc5-c5ccccc5)n4)c3)n2)cc1 ; Fc1ccc(-c2nc(-c3ccccc3)nc(-c3cccc(-c4nc(-c5ccccc5)nc(-c5ccc(-c6ccc(-c7ccccc7)cc6)cc5)n4)c3)n2)cc1 ; Fc1ccc(-c2nc(-c3ccccc3)nc(-c3cccc(-c4nc(-c5ccccc5)nc(-c5ccc(-c6cccc(-c7ccccc7)c6)cc5)n4)c3)n2)cc1]

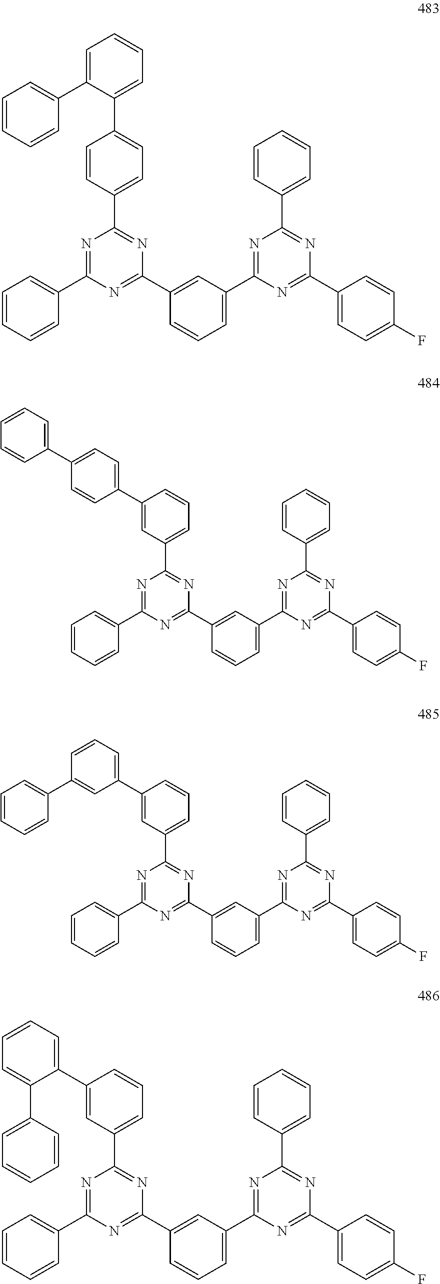 [SMILES: Fc1ccc(-c2nc(-c3ccccc3)nc(-c3cccc(-c4nc(-c5ccccc5)nc(-c5ccc(-c6ccccc6-c6ccccc6)cc5)n4)c3)n2)cc1 ; Fc1ccc(-c2nc(-c3ccccc3)nc(-c3cccc(-c4nc(-c5ccccc5)nc(-c5cccc(-c6ccc(-c7ccccc7)cc6)c5)n4)c3)n2)cc1 ; Fc1ccc(-c2nc(-c3ccccc3)nc(-c3cccc(-c4nc(-c5ccccc5)nc(-c5cccc(-c6cccc(-c7ccccc7)c6)c5)n4)c3)n2)cc1 ; Fc1ccc(-c2nc(-c3ccccc3)nc(-c3cccc(-c4nc(-c5ccccc5)nc(-c5cccc(-c6ccccc6-c6ccccc6)c5)n4)c3)n2)cc1]

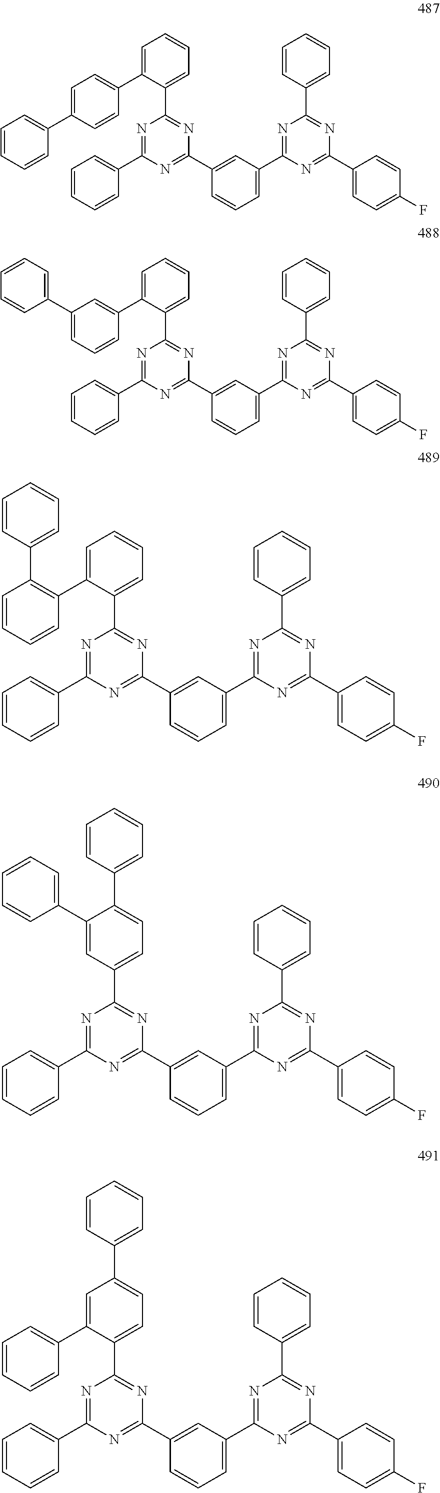 [SMILES: Fc1ccc(-c2nc(-c3ccccc3)nc(-c3cccc(-c4nc(-c5ccccc5)nc(-c5ccccc5-c5ccc(-c6ccccc6)cc5)n4)c3)n2)cc1 ; Fc1ccc(-c2nc(-c3ccccc3)nc(-c3cccc(-c4nc(-c5ccccc5)nc(-c5ccccc5-c5cccc(-c6ccccc6)c5)n4)c3)n2)cc1 ; Fc1ccc(-c2nc(-c3ccccc3)nc(-c3cccc(-c4nc(-c5ccccc5)nc(-c5ccccc5-c5ccccc5-c5ccccc5)n4)c3)n2)cc1 ; Fc1ccc(-c2nc(-c3ccccc3)nc(-c3cccc(-c4nc(-c5ccccc5)nc(-c5ccc(-c6ccccc6)c(-c6ccccc6)c5)n4)c3)n2)cc1 ; Fc1ccc(-c2nc(-c3ccccc3)nc(-c3cccc(-c4nc(-c5ccccc5)nc(-c5ccc(-c6ccccc6)cc5-c5ccccc5)n4)c3)n2)cc1]

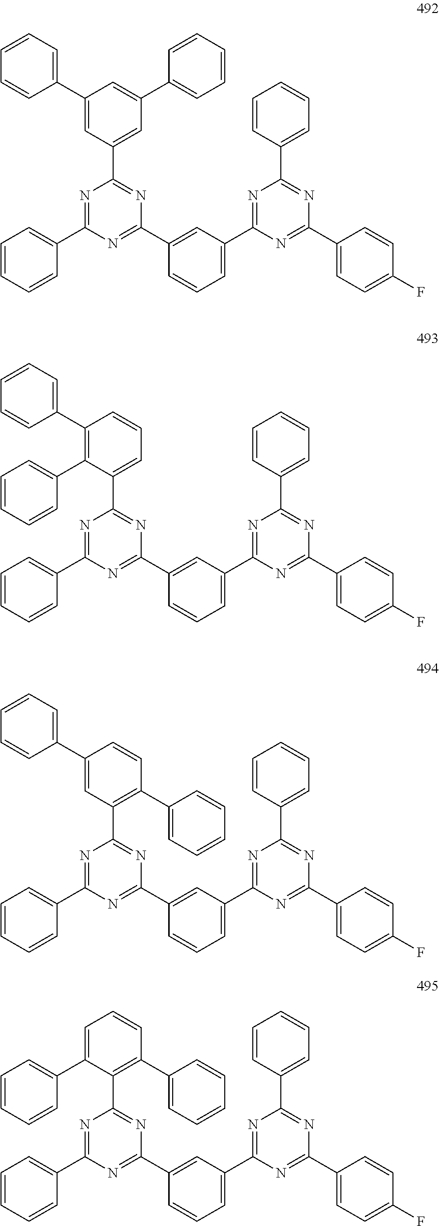 [SMILES: Fc1ccc(-c2nc(-c3ccccc3)nc(-c3cccc(-c4nc(-c5ccccc5)nc(-c5cc(-c6ccccc6)cc(-c6ccccc6)c5)n4)c3)n2)cc1 ; Fc1ccc(-c2nc(-c3ccccc3)nc(-c3cccc(-c4nc(-c5ccccc5)nc(-c5cccc(-c6ccccc6)c5-c5ccccc5)n4)c3)n2)cc1 ; Fc1ccc(-c2nc(-c3ccccc3)nc(-c3cccc(-c4nc(-c5ccccc5)nc(-c5cc(-c6ccccc6)ccc5-c5ccccc5)n4)c3)n2)cc1 ; Fc1ccc(-c2nc(-c3ccccc3)nc(-c3cccc(-c4nc(-c5ccccc5)nc(-c5c(-c6ccccc6)cccc5-c5ccccc5)n4)c3)n2)cc1]

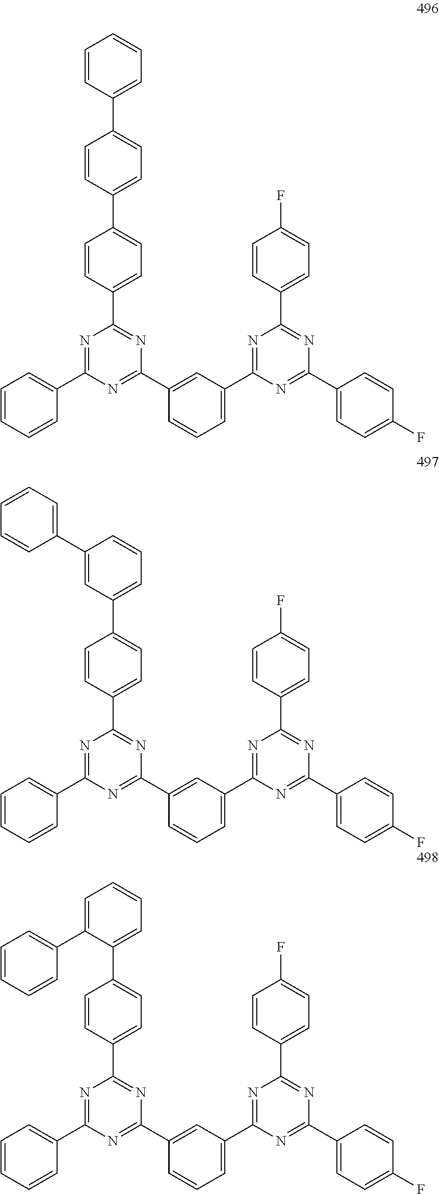 [SMILES: Fc1ccc(-c2nc(-c3ccc(F)cc3)nc(-c3cccc(-c4nc(-c5ccccc5)nc(-c5ccc(-c6ccc(-c7ccccc7)cc6)cc5)n4)c3)n2)cc1 ; Fc1ccc(-c2nc(-c3ccc(F)cc3)nc(-c3cccc(-c4nc(-c5ccccc5)nc(-c5ccc(-c6cccc(-c7ccccc7)c6)cc5)n4)c3)n2)cc1 ; Fc1ccc(-c2nc(-c3ccc(F)cc3)nc(-c3cccc(-c4nc(-c5ccccc5)nc(-c5ccc(-c6ccccc6-c6ccccc6)cc5)n4)c3)n2)cc1]

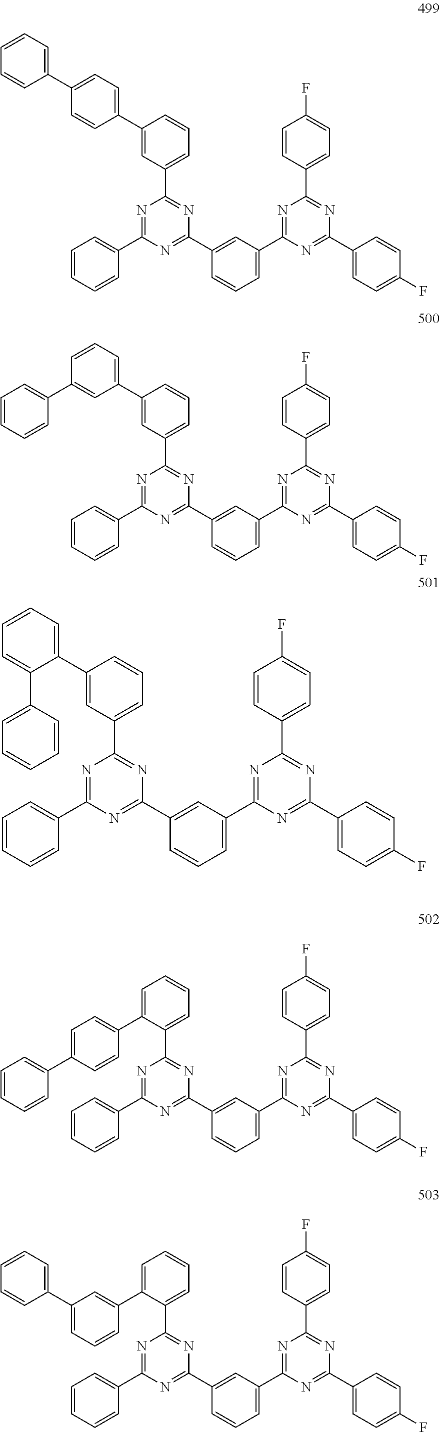

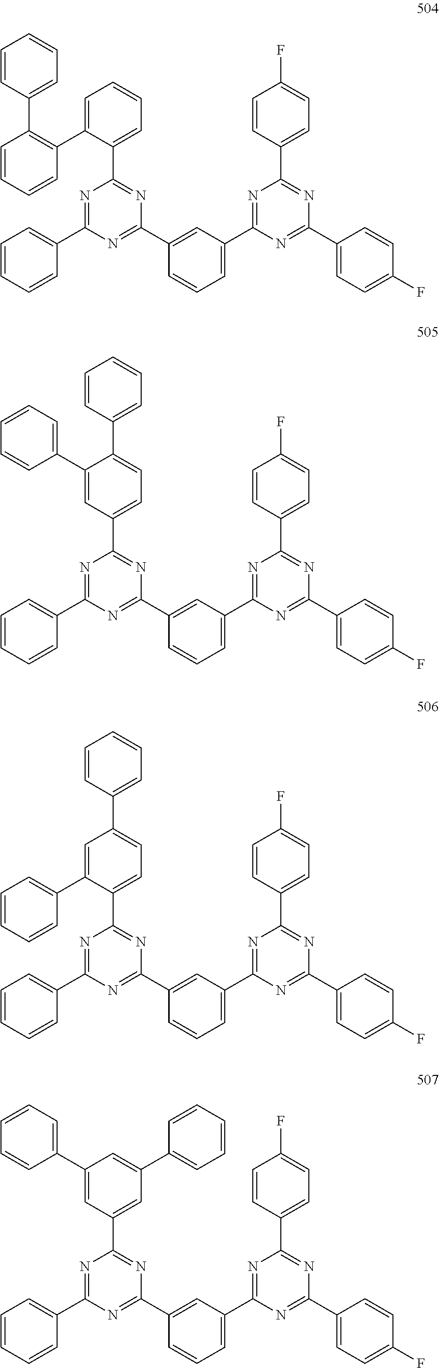 [SMILES: Fc1ccc(-c2nc(-c3ccc(F)cc3)nc(-c3cccc(-c4nc(-c5ccccc5)nc(-c5ccccc5-c5ccccc5-c5ccccc5)n4)c3)n2)cc1 ; Fc1ccc(-c2nc(-c3ccc(F)cc3)nc(-c3cccc(-c4nc(-c5ccccc5)nc(-c5ccc(-c6ccccc6)c(-c6ccccc6)c5)n4)c3)n2)cc1 ; Fc1ccc(-c2nc(-c3ccc(F)cc3)nc(-c3cccc(-c4nc(-c5ccccc5)nc(-c5ccc(-c6ccccc6)cc5-c5ccccc5)n4)c3)n2)cc1 ; Fc1ccc(-c2nc(-c3ccc(F)cc3)nc(-c3cccc(-c4nc(-c5ccccc5)nc(-c5cc(-c6ccccc6)cc(-c6ccccc6)c5)n4)c3)n2)cc1]

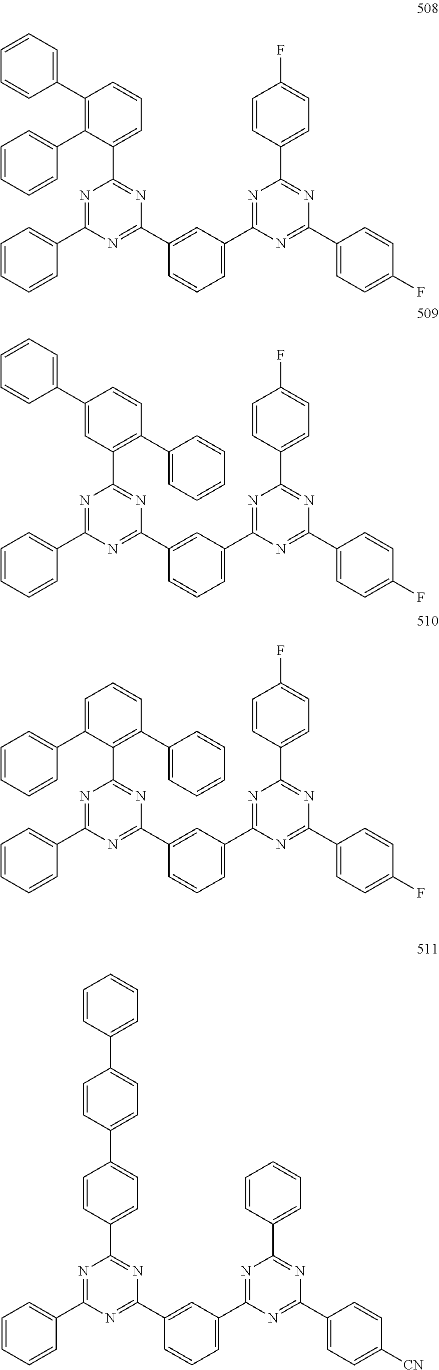 [SMILES: Fc1ccc(-c2nc(-c3ccc(F)cc3)nc(-c3cccc(-c4nc(-c5ccccc5)nc(-c5cccc(-c6ccccc6)c5-c5ccccc5)n4)c3)n2)cc1 ; Fc1ccc(-c2nc(-c3ccc(F)cc3)nc(-c3cccc(-c4nc(-c5ccccc5)nc(-c5cc(-c6ccccc6)ccc5-c5ccccc5)n4)c3)n2)cc1 ; Fc1ccc(-c2nc(-c3ccc(F)cc3)nc(-c3cccc(-c4nc(-c5ccccc5)nc(-c5c(-c6ccccc6)cccc5-c5ccccc5)n4)c3)n2)cc1 ; N#Cc1ccc(-c2nc(-c3ccccc3)nc(-c3cccc(-c4nc(-c5ccccc5)nc(-c5ccc(-c6ccc(-c7ccccc7)cc6)cc5)n4)c3)n2)cc1]

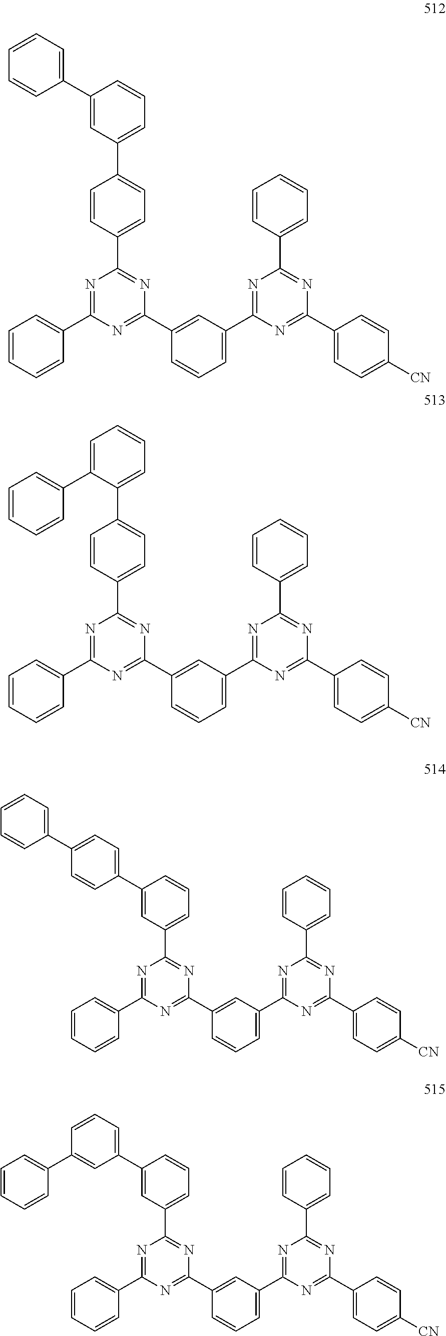 [SMILES: N#Cc1ccc(-c2nc(-c3ccccc3)nc(-c3cccc(-c4nc(-c5ccccc5)nc(-c5ccc(-c6cccc(-c7ccccc7)c6)cc5)n4)c3)n2)cc1 ; N#Cc1ccc(-c2nc(-c3ccccc3)nc(-c3cccc(-c4nc(-c5ccccc5)nc(-c5ccc(-c6ccccc6-c6ccccc6)cc5)n4)c3)n2)cc1 ; N#Cc1ccc(-c2nc(-c3ccccc3)nc(-c3cccc(-c4nc(-c5ccccc5)nc(-c5cccc(-c6ccc(-c7ccccc7)cc6)c5)n4)c3)n2)cc1 ; N#Cc1ccc(-c2nc(-c3ccccc3)nc(-c3cccc(-c4nc(-c5ccccc5)nc(-c5cccc(-c6cccc(-c7ccccc7)c6)c5)n4)c3)n2)cc1]

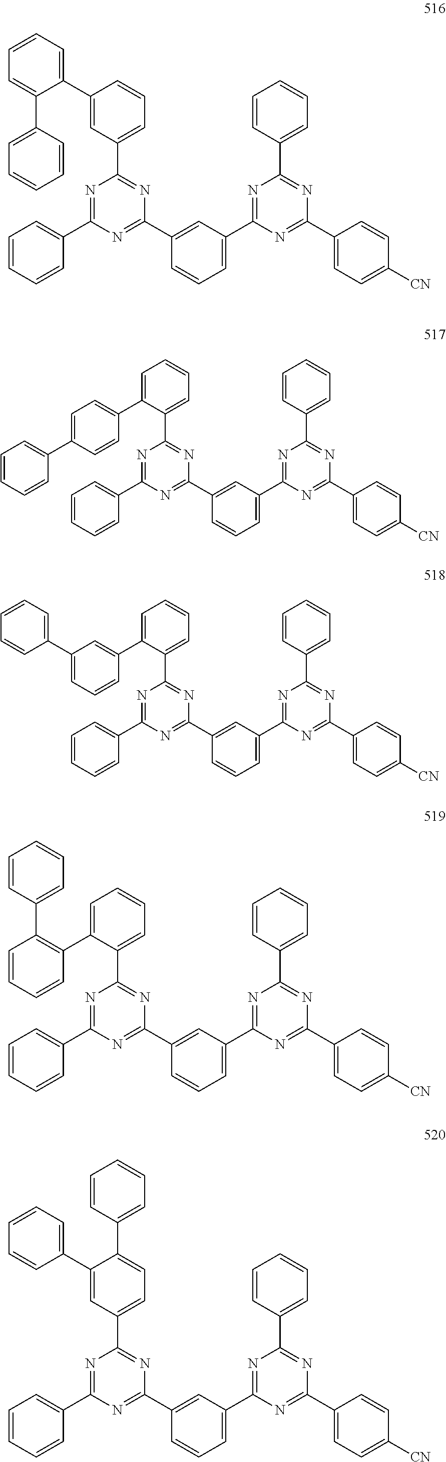 [SMILES: N#Cc1ccc(-c2nc(-c3ccccc3)nc(-c3cccc(-c4nc(-c5ccccc5)nc(-c5cccc(-c6ccccc6-c6ccccc6)c5)n4)c3)n2)cc1 ; N#Cc1ccc(-c2nc(-c3ccccc3)nc(-c3cccc(-c4nc(-c5ccccc5)nc(-c5ccccc5-c5ccc(-c6ccccc6)cc5)n4)c3)n2)cc1 ; N#Cc1ccc(-c2nc(-c3ccccc3)nc(-c3cccc(-c4nc(-c5ccccc5)nc(-c5ccccc5-c5cccc(-c6ccccc6)c5)n4)c3)n2)cc1 ; N#Cc1ccc(-c2nc(-c3ccccc3)nc(-c3cccc(-c4nc(-c5ccccc5)nc(-c5ccccc5-c5ccccc5-c5ccccc5)n4)c3)n2)cc1 ; N#Cc1ccc(-c2nc(-c3ccccc3)nc(-c3cccc(-c4nc(-c5ccccc5)nc(-c5ccc(-c6ccccc6)c(-c6ccccc6)c5)n4)c3)n2)cc1]

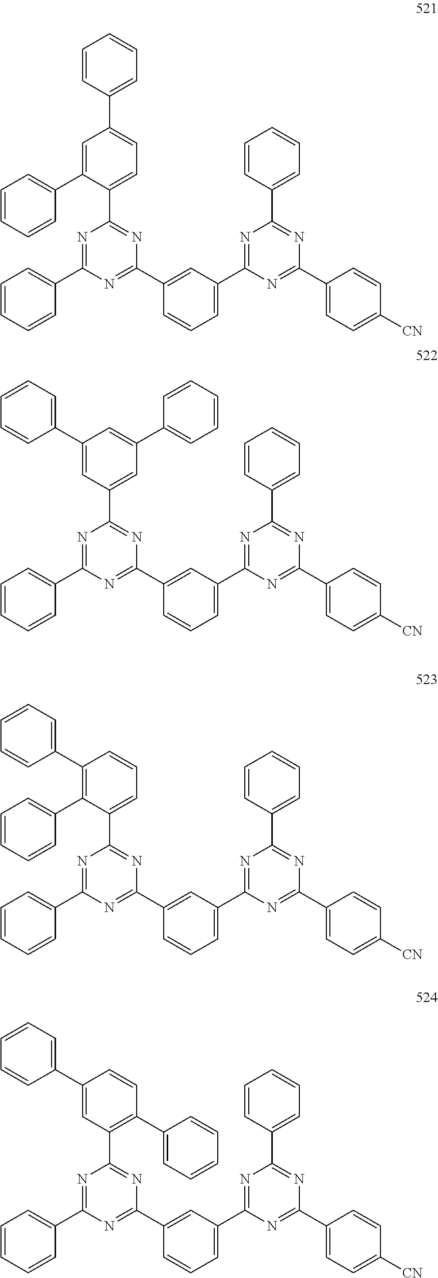 [SMILES: N#Cc1ccc(-c2nc(-c3ccccc3)nc(-c3cccc(-c4nc(-c5ccccc5)nc(-c5ccc(-c6ccccc6)cc5-c5ccccc5)n4)c3)n2)cc1 ; N#Cc1ccc(-c2nc(-c3ccccc3)nc(-c3cccc(-c4nc(-c5ccccc5)nc(-c5cc(-c6ccccc6)cc(-c6ccccc6)c5)n4)c3)n2)cc1 ; N#Cc1ccc(-c2nc(-c3ccccc3)nc(-c3cccc(-c4nc(-c5ccccc5)nc(-c5cccc(-c6ccccc6)c5-c5ccccc5)n4)c3)n2)cc1 ; N#Cc1ccc(-c2nc(-c3ccccc3)nc(-c3cccc(-c4nc(-c5ccccc5)nc(-c5cc(-c6ccccc6)ccc5-c5ccccc5)n4)c3)n2)cc1]

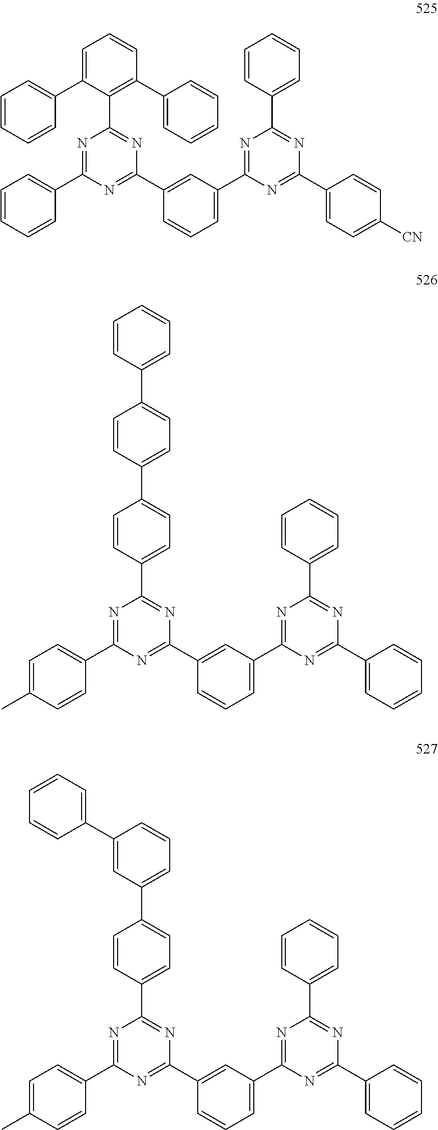 [SMILES: N#Cc1ccc(-c2nc(-c3ccccc3)nc(-c3cccc(-c4nc(-c5ccccc5)nc(-c5c(-c6ccccc6)cccc5-c5ccccc5)n4)c3)n2)cc1 ; Cc1ccc(-c2nc(-c3ccc(-c4ccc(-c5ccccc5)cc4)cc3)nc(-c3cccc(-c4nc(-c5ccccc5)nc(-c5ccccc5)n4)c3)n2)cc1 ; Cc1ccc(-c2nc(-c3ccc(-c4cccc(-c5ccccc5)c4)cc3)nc(-c3cccc(-c4nc(-c5ccccc5)nc(-c5ccccc5)n4)c3)n2)cc1]

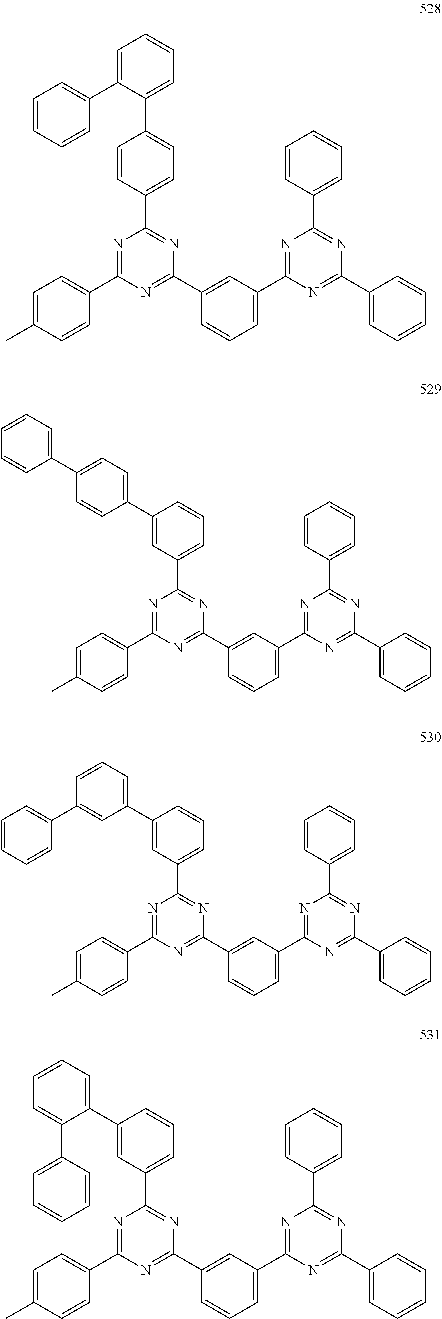 [SMILES: Cc1ccc(-c2nc(-c3ccc(-c4ccccc4-c4ccccc4)cc3)nc(-c3cccc(-c4nc(-c5ccccc5)nc(-c5ccccc5)n4)c3)n2)cc1 ; Cc1ccc(-c2nc(-c3cccc(-c4ccc(-c5ccccc5)cc4)c3)nc(-c3cccc(-c4nc(-c5ccccc5)nc(-c5ccccc5)n4)c3)n2)cc1 ; Cc1ccc(-c2nc(-c3cccc(-c4cccc(-c5ccccc5)c4)c3)nc(-c3cccc(-c4nc(-c5ccccc5)nc(-c5ccccc5)n4)c3)n2)cc1 ; Cc1ccc(-c2nc(-c3cccc(-c4nc(-c5ccccc5)nc(-c5ccccc5)n4)c3)nc(-c3cccc(-c4ccccc4-c4ccccc4)c3)n2)cc1]

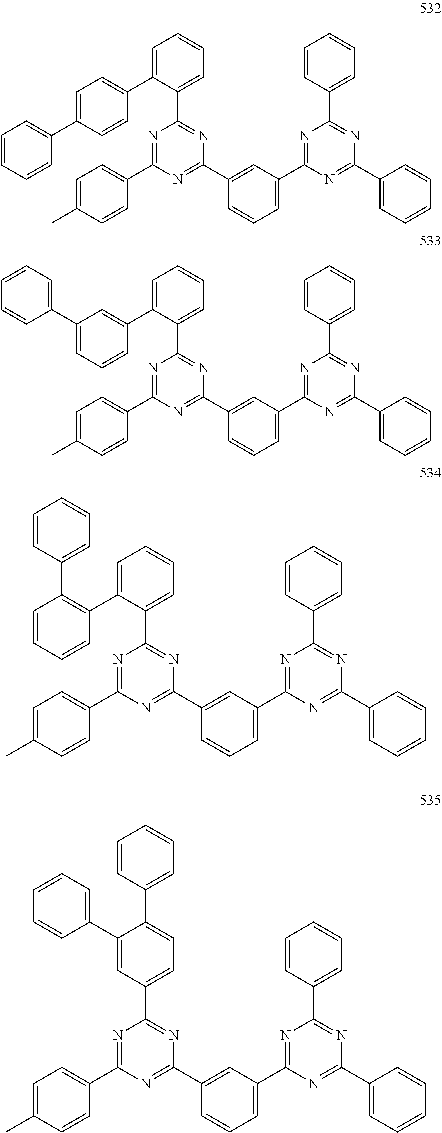 [SMILES: Cc1ccc(-c2nc(-c3cccc(-c4nc(-c5ccccc5)nc(-c5ccccc5)n4)c3)nc(-c3ccccc3-c3ccc(-c4ccccc4)cc3)n2)cc1 ; Cc1ccc(-c2nc(-c3cccc(-c4nc(-c5ccccc5)nc(-c5ccccc5)n4)c3)nc(-c3ccccc3-c3cccc(-c4ccccc4)c3)n2)cc1 ; Cc1ccc(-c2nc(-c3cccc(-c4nc(-c5ccccc5)nc(-c5ccccc5)n4)c3)nc(-c3ccccc3-c3ccccc3-c3ccccc3)n2)cc1 ; Cc1ccc(-c2nc(-c3cccc(-c4nc(-c5ccccc5)nc(-c5ccccc5)n4)c3)nc(-c3ccc(-c4ccccc4)c(-c4ccccc4)c3)n2)cc1]

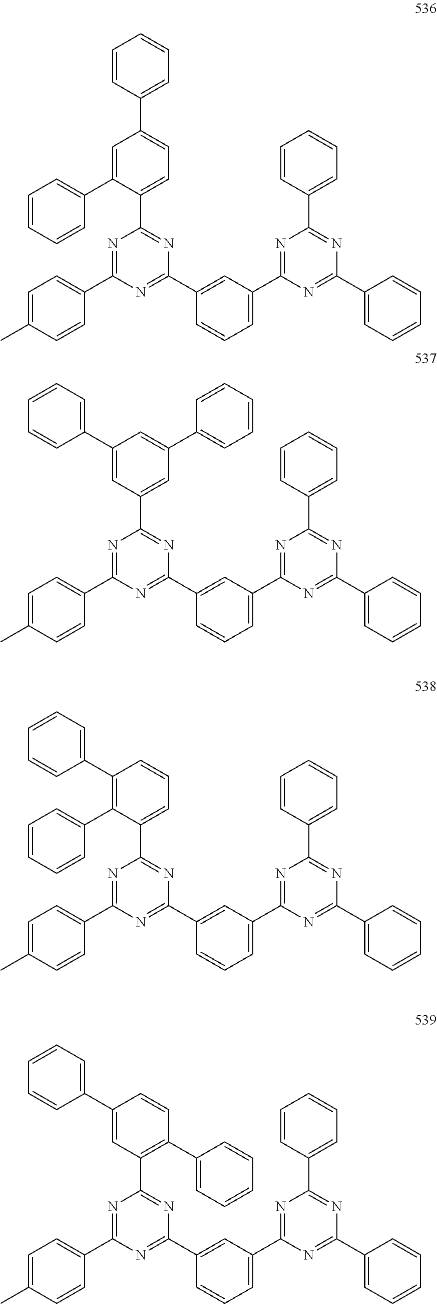

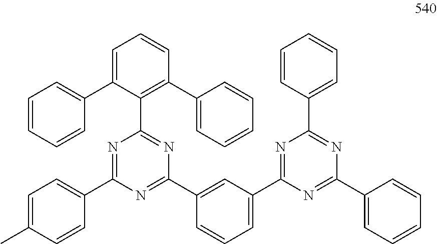 [SMILES: Cc1ccc(-c2nc(-c3cccc(-c4nc(-c5ccccc5)nc(-c5ccccc5)n4)c3)nc(-c3c(-c4ccccc4)cccc3-c3ccccc3)n2)cc1]

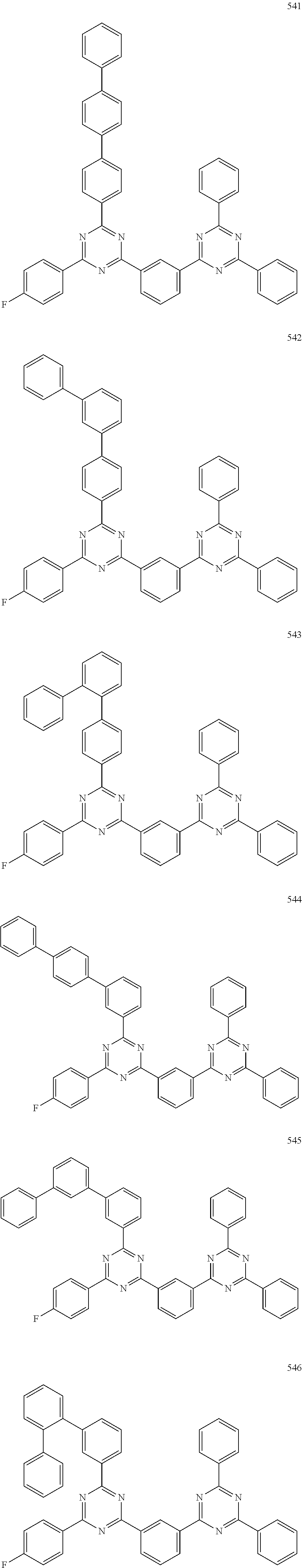

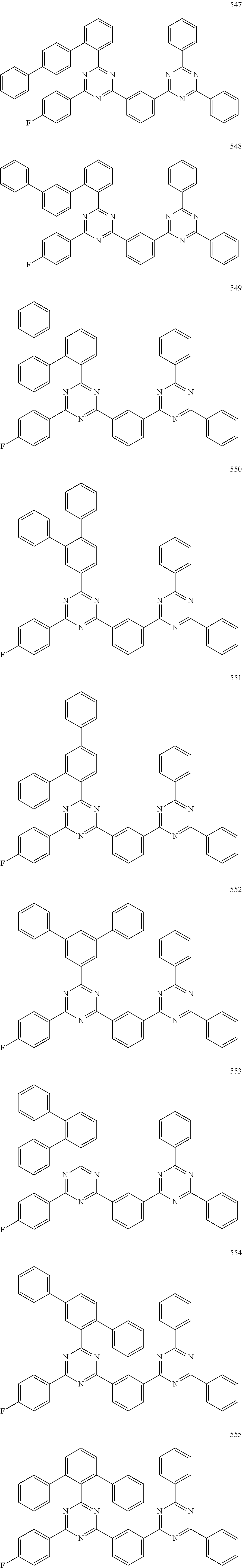

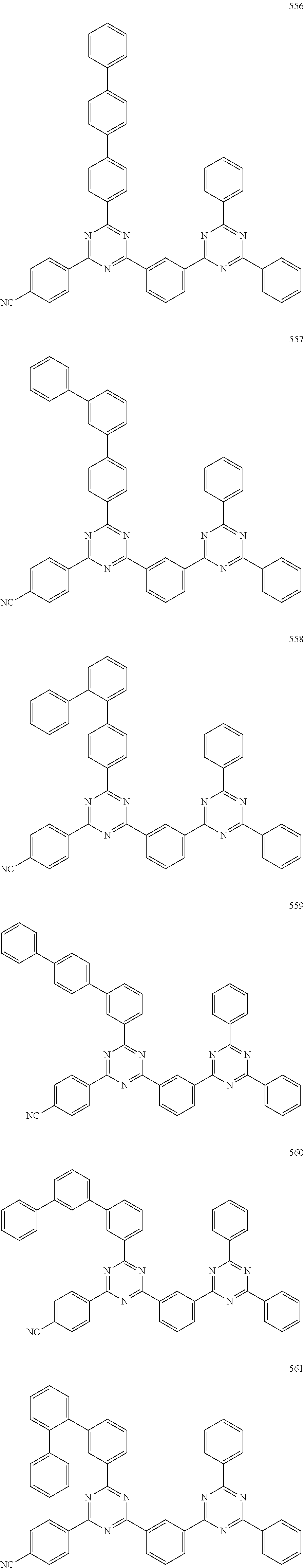

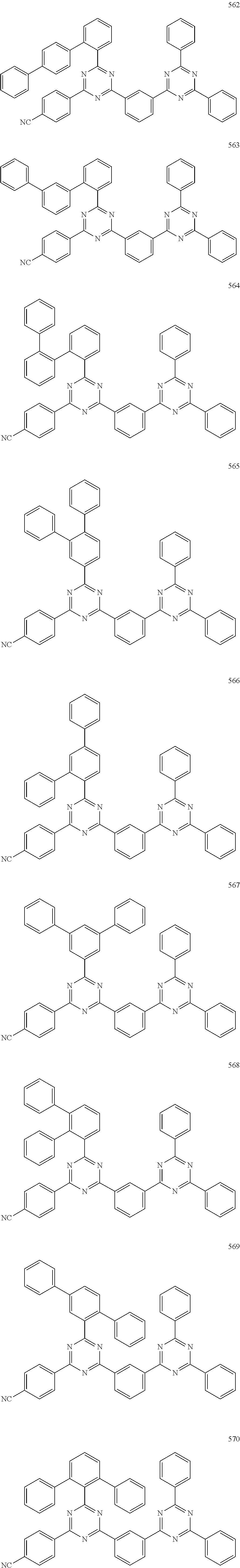

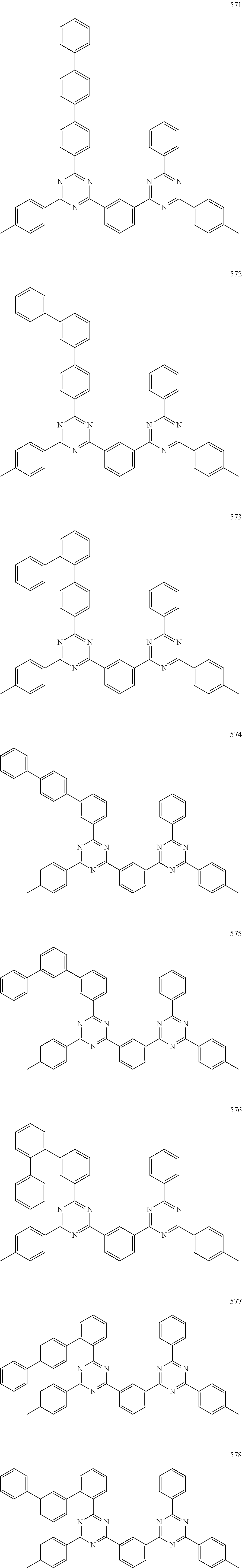

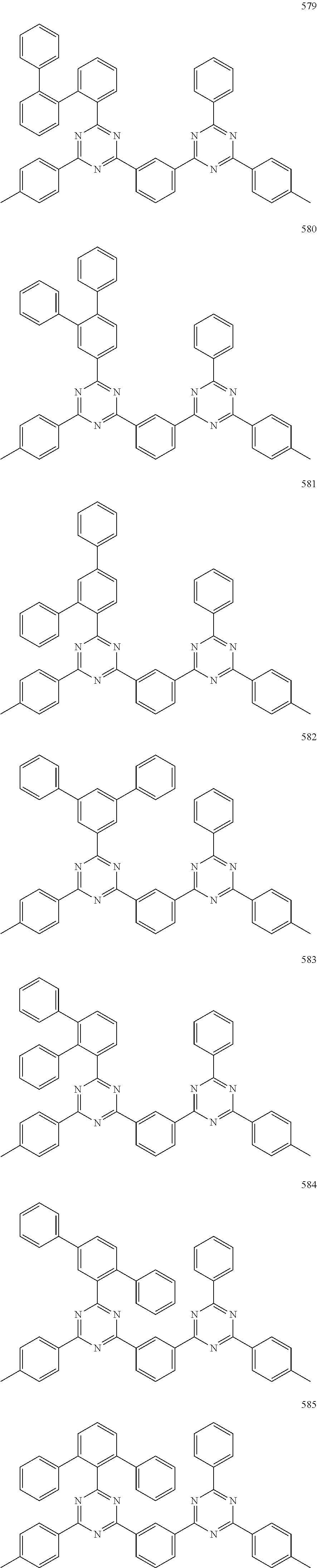

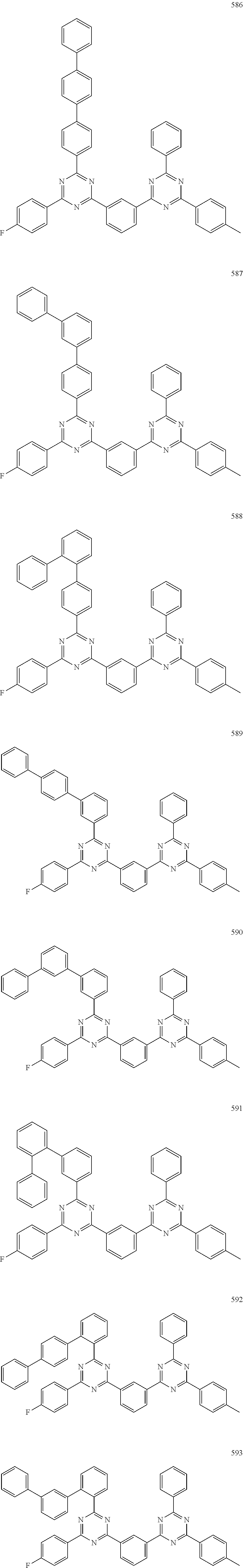

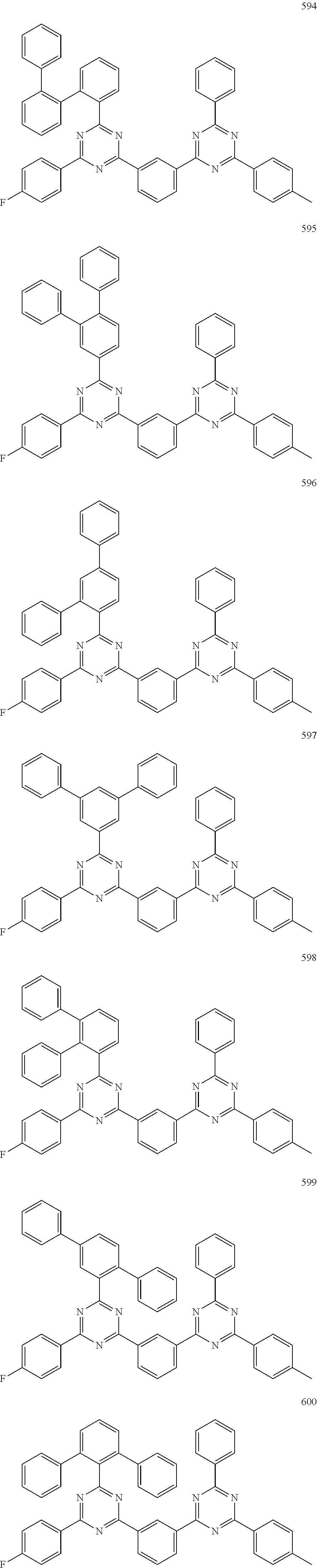

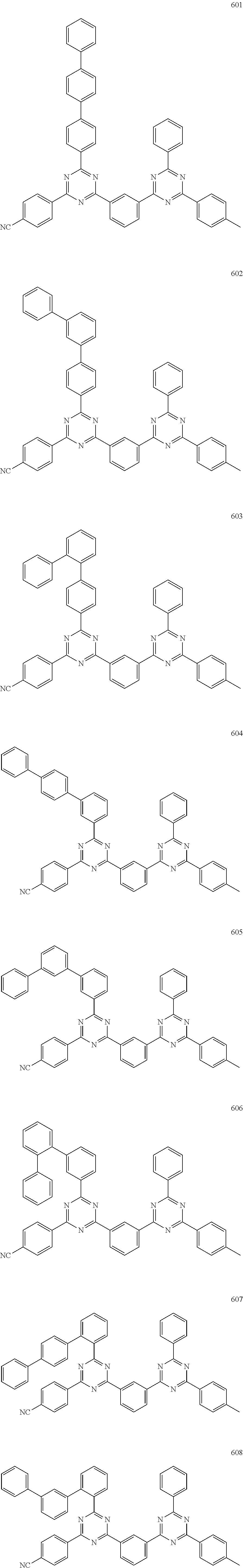

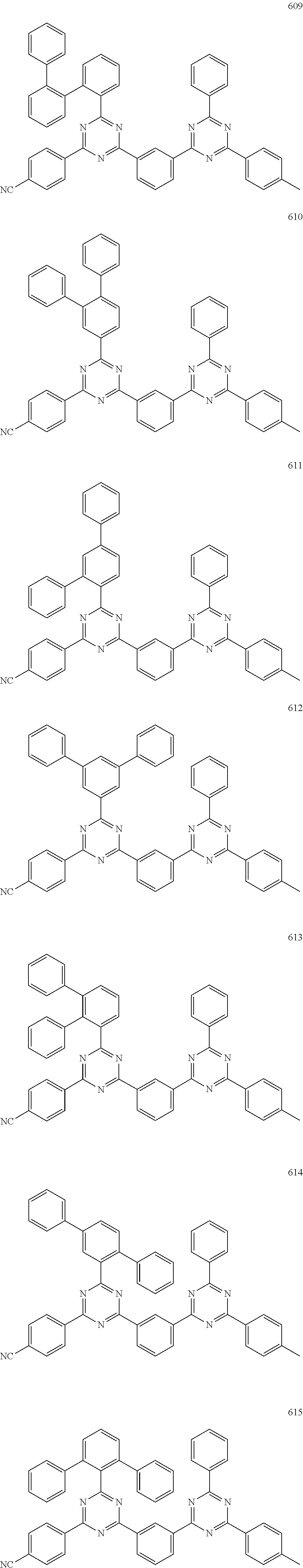

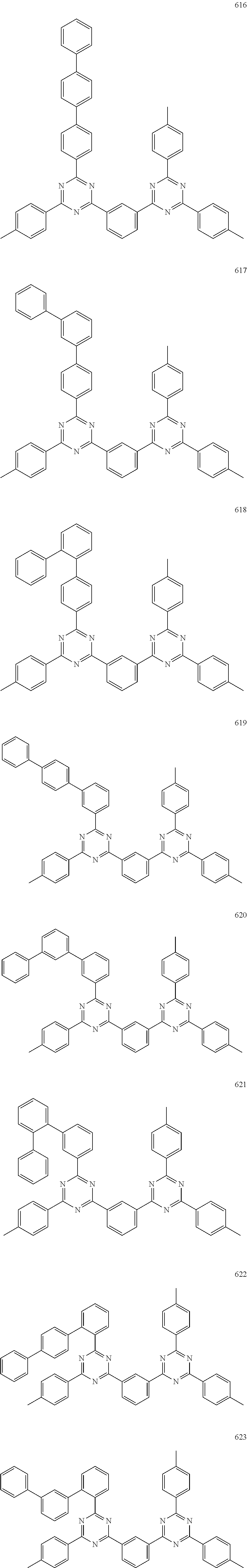

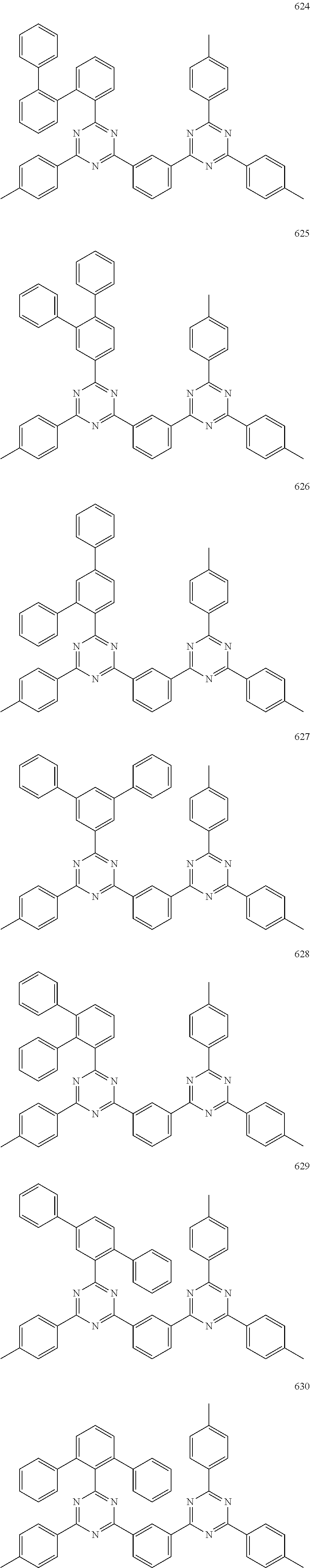

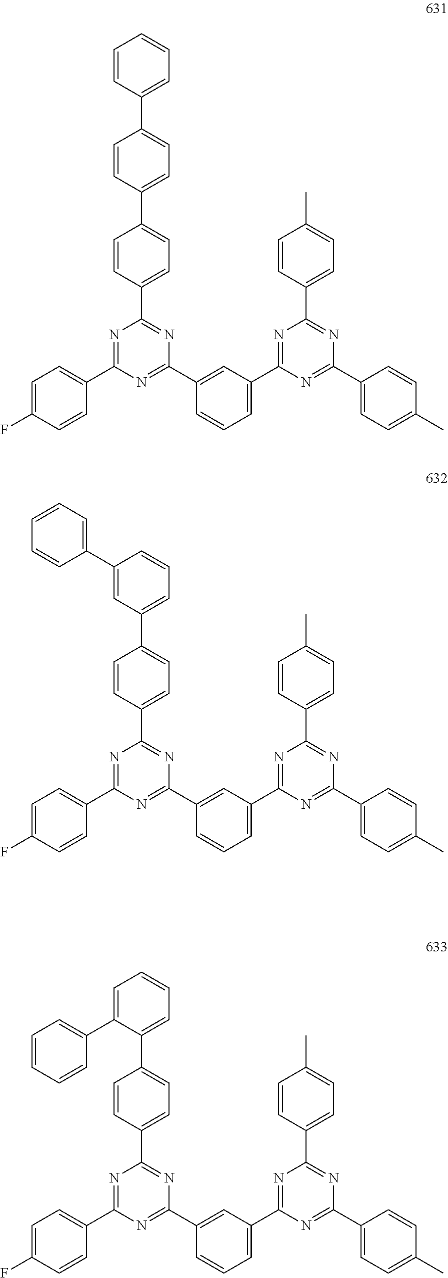 [SMILES: Cc1ccc(-c2nc(-c3ccc(C)cc3)nc(-c3cccc(-c4nc(-c5ccc(F)cc5)nc(-c5ccc(-c6ccc(-c7ccccc7)cc6)cc5)n4)c3)n2)cc1 ; Cc1ccc(-c2nc(-c3ccc(C)cc3)nc(-c3cccc(-c4nc(-c5ccc(F)cc5)nc(-c5ccc(-c6cccc(-c7ccccc7)c6)cc5)n4)c3)n2)cc1 ; Cc1ccc(-c2nc(-c3ccc(C)cc3)nc(-c3cccc(-c4nc(-c5ccc(F)cc5)nc(-c5ccc(-c6ccccc6-c6ccccc6)cc5)n4)c3)n2)cc1]

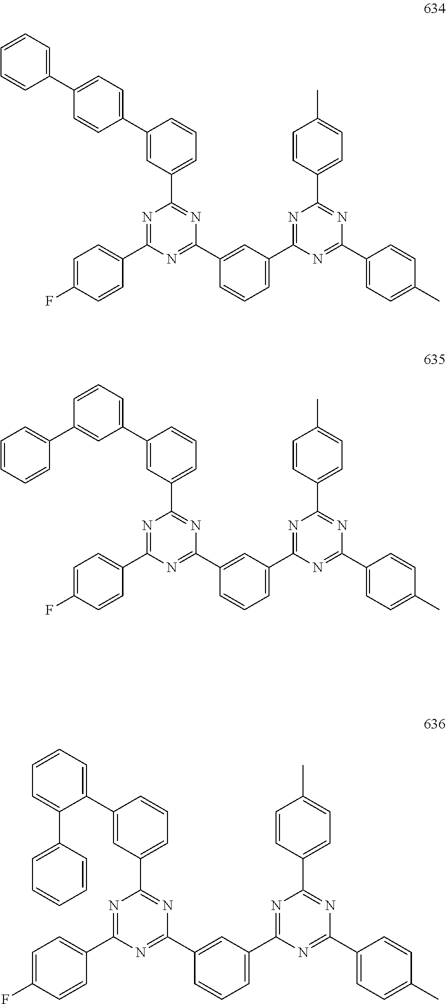 [SMILES: Cc1ccc(-c2nc(-c3ccc(C)cc3)nc(-c3cccc(-c4nc(-c5ccc(F)cc5)nc(-c5cccc(-c6ccc(-c7ccccc7)cc6)c5)n4)c3)n2)cc1 ; Cc1ccc(-c2nc(-c3ccc(C)cc3)nc(-c3cccc(-c4nc(-c5ccc(F)cc5)nc(-c5cccc(-c6cccc(-c7ccccc7)c6)c5)n4)c3)n2)cc1 ; Cc1ccc(-c2nc(-c3ccc(C)cc3)nc(-c3cccc(-c4nc(-c5ccc(F)cc5)nc(-c5cccc(-c6ccccc6-c6ccccc6)c5)n4)c3)n2)cc1]

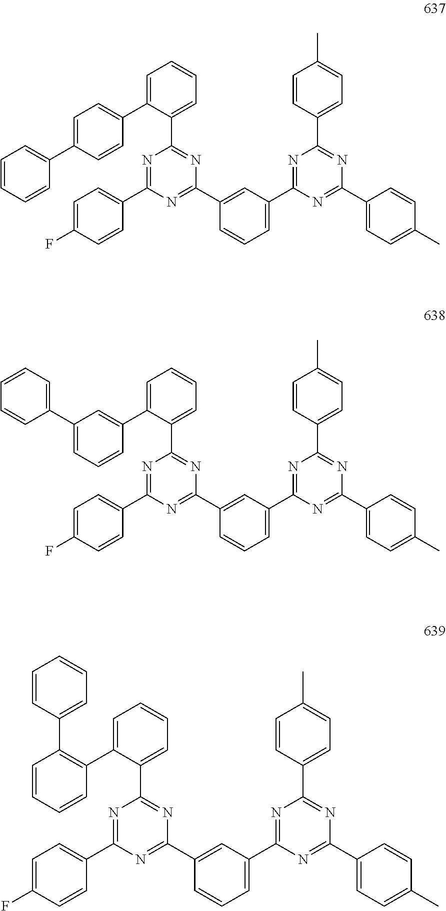 [SMILES: Cc1ccc(-c2nc(-c3ccc(C)cc3)nc(-c3cccc(-c4nc(-c5ccc(F)cc5)nc(-c5ccccc5-c5ccc(-c6ccccc6)cc5)n4)c3)n2)cc1 ; Cc1ccc(-c2nc(-c3ccc(C)cc3)nc(-c3cccc(-c4nc(-c5ccc(F)cc5)nc(-c5ccccc5-c5cccc(-c6ccccc6)c5)n4)c3)n2)cc1 ; Cc1ccc(-c2nc(-c3ccc(C)cc3)nc(-c3cccc(-c4nc(-c5ccc(F)cc5)nc(-c5ccccc5-c5ccccc5-c5ccccc5)n4)c3)n2)cc1]

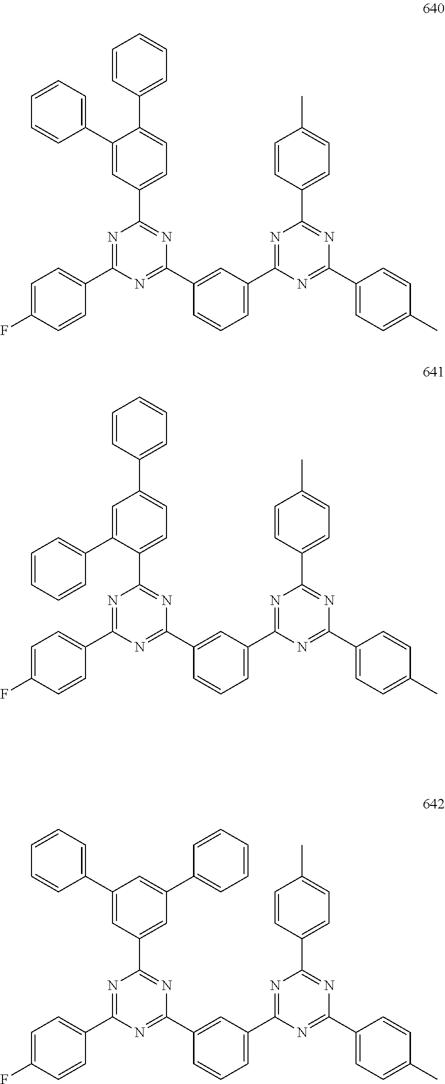 [SMILES: Cc1ccc(-c2nc(-c3ccc(C)cc3)nc(-c3cccc(-c4nc(-c5ccc(F)cc5)nc(-c5ccc(-c6ccccc6)c(-c6ccccc6)c5)n4)c3)n2)cc1 ; Cc1ccc(-c2nc(-c3ccc(C)cc3)nc(-c3cccc(-c4nc(-c5ccc(F)cc5)nc(-c5ccc(-c6ccccc6)cc5-c5ccccc5)n4)c3)n2)cc1 ; Cc1ccc(-c2nc(-c3ccc(C)cc3)nc(-c3cccc(-c4nc(-c5ccc(F)cc5)nc(-c5cc(-c6ccccc6)cc(-c6ccccc6)c5)n4)c3)n2)cc1]

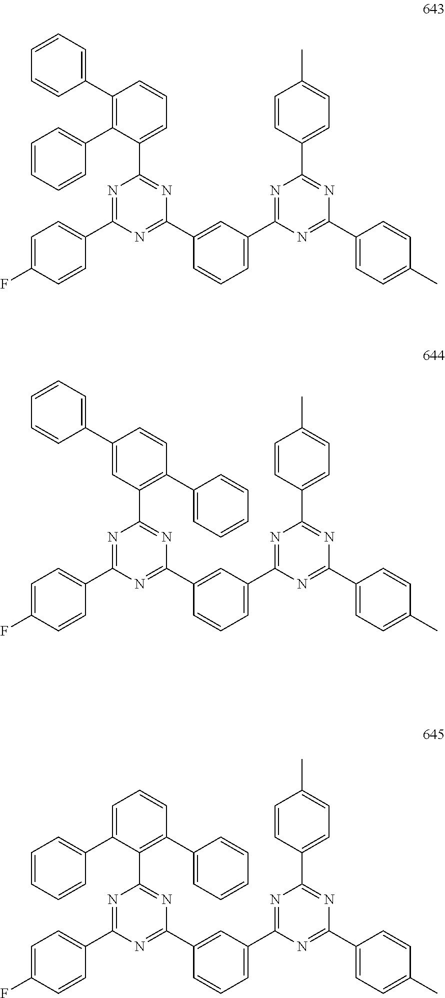 [SMILES: Cc1ccc(-c2nc(-c3ccc(C)cc3)nc(-c3cccc(-c4nc(-c5ccc(F)cc5)nc(-c5cccc(-c6ccccc6)c5-c5ccccc5)n4)c3)n2)cc1 ; Cc1ccc(-c2nc(-c3ccc(C)cc3)nc(-c3cccc(-c4nc(-c5ccc(F)cc5)nc(-c5cc(-c6ccccc6)ccc5-c5ccccc5)n4)c3)n2)cc1 ; Cc1ccc(-c2nc(-c3ccc(C)cc3)nc(-c3cccc(-c4nc(-c5ccc(F)cc5)nc(-c5c(-c6ccccc6)cccc5-c5ccccc5)n4)c3)n2)cc1]

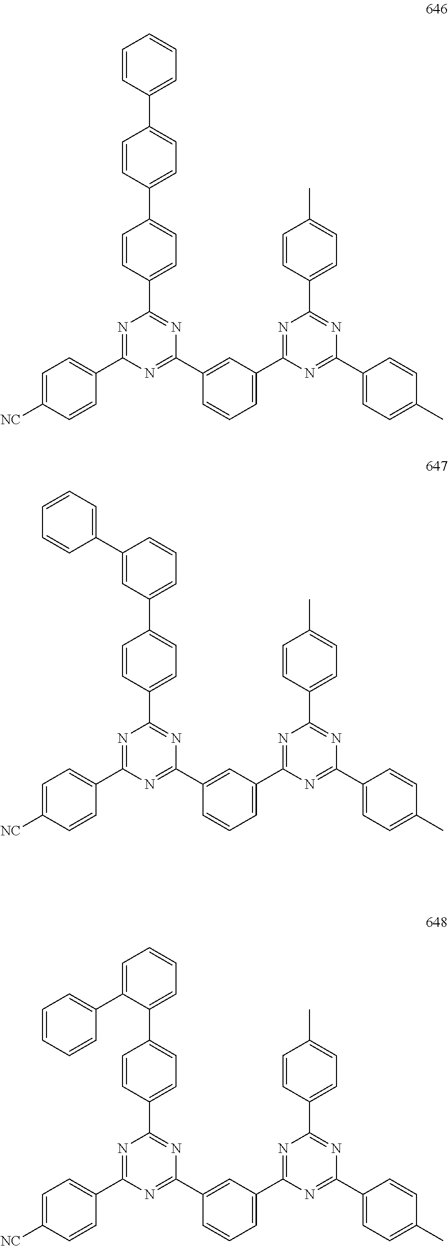 [SMILES: Cc1ccc(-c2nc(-c3ccc(C)cc3)nc(-c3cccc(-c4nc(-c5ccc(C#N)cc5)nc(-c5ccc(-c6ccc(-c7ccccc7)cc6)cc5)n4)c3)n2)cc1 ; Cc1ccc(-c2nc(-c3ccc(C)cc3)nc(-c3cccc(-c4nc(-c5ccc(C#N)cc5)nc(-c5ccc(-c6cccc(-c7ccccc7)c6)cc5)n4)c3)n2)cc1 ; Cc1ccc(-c2nc(-c3ccc(C)cc3)nc(-c3cccc(-c4nc(-c5ccc(C#N)cc5)nc(-c5ccc(-c6ccccc6-c6ccccc6)cc5)n4)c3)n2)cc1]

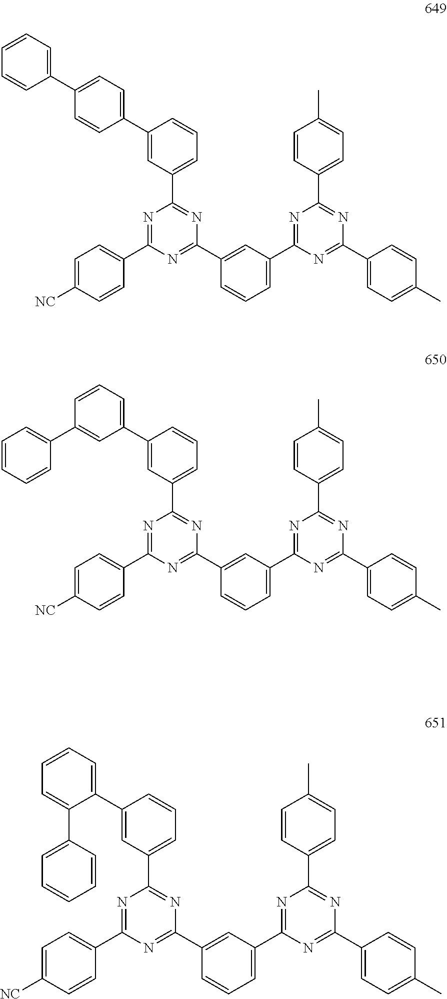 [SMILES: Cc1ccc(-c2nc(-c3ccc(C)cc3)nc(-c3cccc(-c4nc(-c5ccc(C#N)cc5)nc(-c5cccc(-c6ccc(-c7ccccc7)cc6)c5)n4)c3)n2)cc1 ; Cc1ccc(-c2nc(-c3ccc(C)cc3)nc(-c3cccc(-c4nc(-c5ccc(C#N)cc5)nc(-c5cccc(-c6cccc(-c7ccccc7)c6)c5)n4)c3)n2)cc1 ; Cc1ccc(-c2nc(-c3ccc(C)cc3)nc(-c3cccc(-c4nc(-c5ccc(C#N)cc5)nc(-c5cccc(-c6ccccc6-c6ccccc6)c5)n4)c3)n2)cc1]

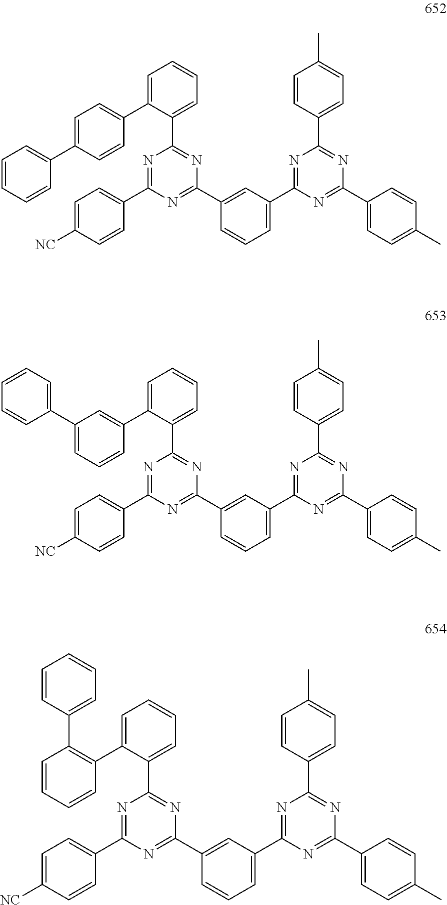 [SMILES: Cc1ccc(-c2nc(-c3ccc(C)cc3)nc(-c3cccc(-c4nc(-c5ccc(C#N)cc5)nc(-c5ccccc5-c5ccc(-c6ccccc6)cc5)n4)c3)n2)cc1 ; Cc1ccc(-c2nc(-c3ccc(C)cc3)nc(-c3cccc(-c4nc(-c5ccc(C#N)cc5)nc(-c5ccccc5-c5cccc(-c6ccccc6)c5)n4)c3)n2)cc1 ; Cc1ccc(-c2nc(-c3ccc(C)cc3)nc(-c3cccc(-c4nc(-c5ccc(C#N)cc5)nc(-c5ccccc5-c5ccccc5-c5ccccc5)n4)c3)n2)cc1]

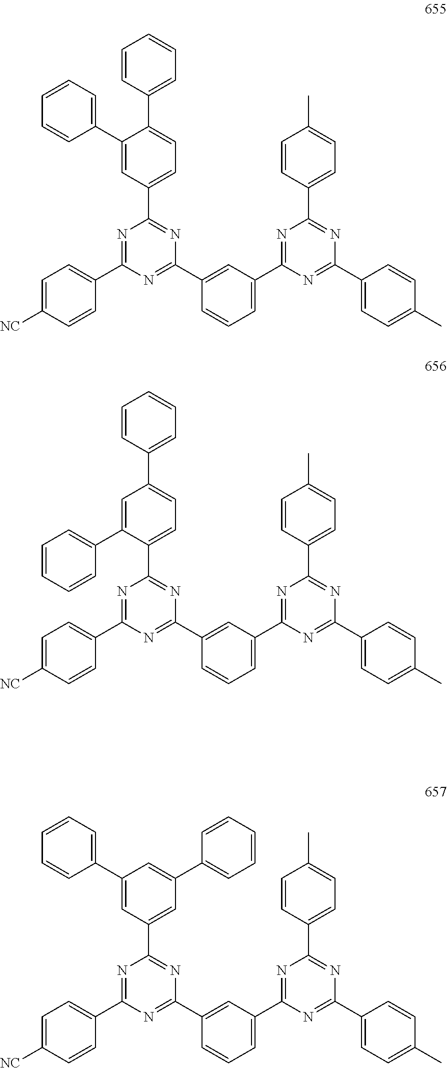 [SMILES: Cc1ccc(-c2nc(-c3ccc(C)cc3)nc(-c3cccc(-c4nc(-c5ccc(C#N)cc5)nc(-c5ccc(-c6ccccc6)c(-c6ccccc6)c5)n4)c3)n2)cc1 ; Cc1ccc(-c2nc(-c3ccc(C)cc3)nc(-c3cccc(-c4nc(-c5ccc(C#N)cc5)nc(-c5ccc(-c6ccccc6)cc5-c5ccccc5)n4)c3)n2)cc1 ; Cc1ccc(-c2nc(-c3ccc(C)cc3)nc(-c3cccc(-c4nc(-c5ccc(C#N)cc5)nc(-c5cc(-c6ccccc6)cc(-c6ccccc6)c5)n4)c3)n2)cc1]

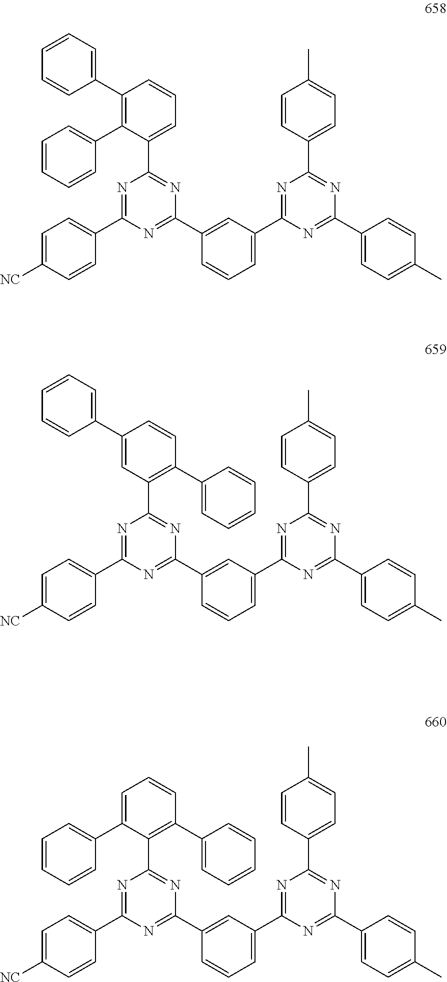 [SMILES: Cc1ccc(-c2nc(-c3ccc(C)cc3)nc(-c3cccc(-c4nc(-c5ccc(C#N)cc5)nc(-c5cccc(-c6ccccc6)c5-c5ccccc5)n4)c3)n2)cc1 ; Cc1ccc(-c2nc(-c3ccc(C)cc3)nc(-c3cccc(-c4nc(-c5ccc(C#N)cc5)nc(-c5cc(-c6ccccc6)ccc5-c5ccccc5)n4)c3)n2)cc1 ; Cc1ccc(-c2nc(-c3ccc(C)cc3)nc(-c3cccc(-c4nc(-c5ccc(C#N)cc5)nc(-c5c(-c6ccccc6)cccc5-c5ccccc5)n4)c3)n2)cc1]

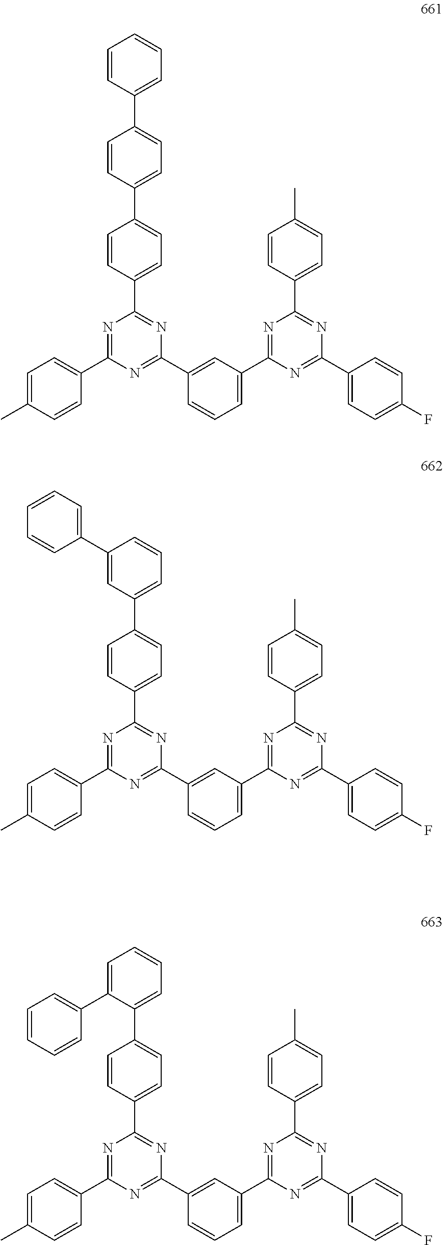 [SMILES: Cc1ccc(-c2nc(-c3ccc(F)cc3)nc(-c3cccc(-c4nc(-c5ccc(C)cc5)nc(-c5ccc(-c6ccc(-c7ccccc7)cc6)cc5)n4)c3)n2)cc1 ; Cc1ccc(-c2nc(-c3ccc(F)cc3)nc(-c3cccc(-c4nc(-c5ccc(C)cc5)nc(-c5ccc(-c6cccc(-c7ccccc7)c6)cc5)n4)c3)n2)cc1 ; Cc1ccc(-c2nc(-c3ccc(F)cc3)nc(-c3cccc(-c4nc(-c5ccc(C)cc5)nc(-c5ccc(-c6ccccc6-c6ccccc6)cc5)n4)c3)n2)cc1]

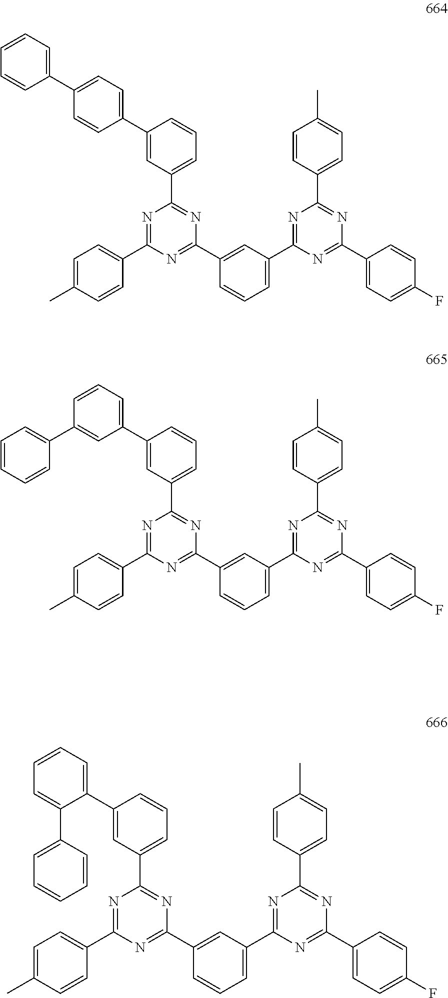 [SMILES: Cc1ccc(-c2nc(-c3ccc(F)cc3)nc(-c3cccc(-c4nc(-c5ccc(C)cc5)nc(-c5cccc(-c6ccc(-c7ccccc7)cc6)c5)n4)c3)n2)cc1 ; Cc1ccc(-c2nc(-c3ccc(F)cc3)nc(-c3cccc(-c4nc(-c5ccc(C)cc5)nc(-c5cccc(-c6cccc(-c7ccccc7)c6)c5)n4)c3)n2)cc1 ; Cc1ccc(-c2nc(-c3ccc(F)cc3)nc(-c3cccc(-c4nc(-c5ccc(C)cc5)nc(-c5cccc(-c6ccccc6-c6ccccc6)c5)n4)c3)n2)cc1]

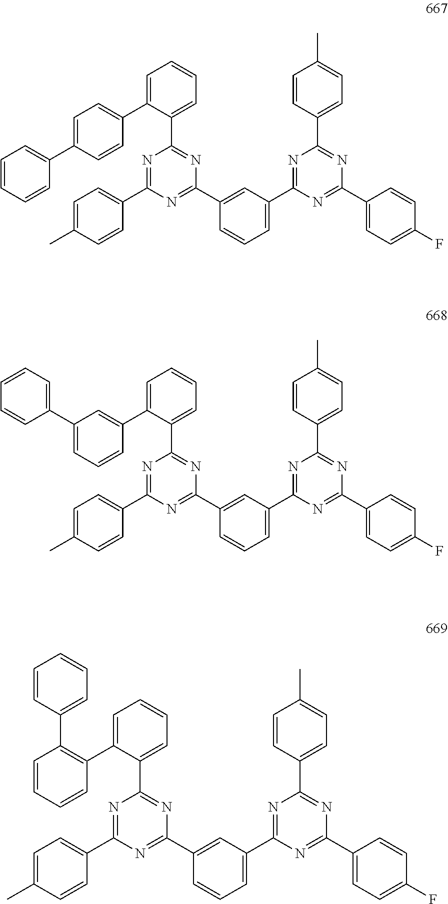 [SMILES: Cc1ccc(-c2nc(-c3ccc(F)cc3)nc(-c3cccc(-c4nc(-c5ccc(C)cc5)nc(-c5ccccc5-c5ccc(-c6ccccc6)cc5)n4)c3)n2)cc1 ; Cc1ccc(-c2nc(-c3ccc(F)cc3)nc(-c3cccc(-c4nc(-c5ccc(C)cc5)nc(-c5ccccc5-c5cccc(-c6ccccc6)c5)n4)c3)n2)cc1 ; Cc1ccc(-c2nc(-c3ccc(F)cc3)nc(-c3cccc(-c4nc(-c5ccc(C)cc5)nc(-c5ccccc5-c5ccccc5-c5ccccc5)n4)c3)n2)cc1]

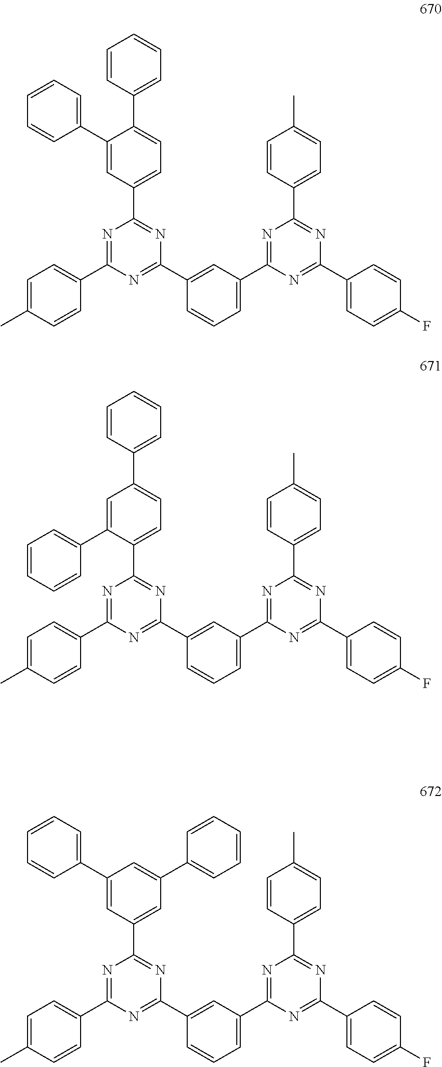 [SMILES: Cc1ccc(-c2nc(-c3ccc(F)cc3)nc(-c3cccc(-c4nc(-c5ccc(C)cc5)nc(-c5ccc(-c6ccccc6)c(-c6ccccc6)c5)n4)c3)n2)cc1 ; Cc1ccc(-c2nc(-c3ccc(F)cc3)nc(-c3cccc(-c4nc(-c5ccc(C)cc5)nc(-c5ccc(-c6ccccc6)cc5-c5ccccc5)n4)c3)n2)cc1 ; Cc1ccc(-c2nc(-c3ccc(F)cc3)nc(-c3cccc(-c4nc(-c5ccc(C)cc5)nc(-c5cc(-c6ccccc6)cc(-c6ccccc6)c5)n4)c3)n2)cc1]

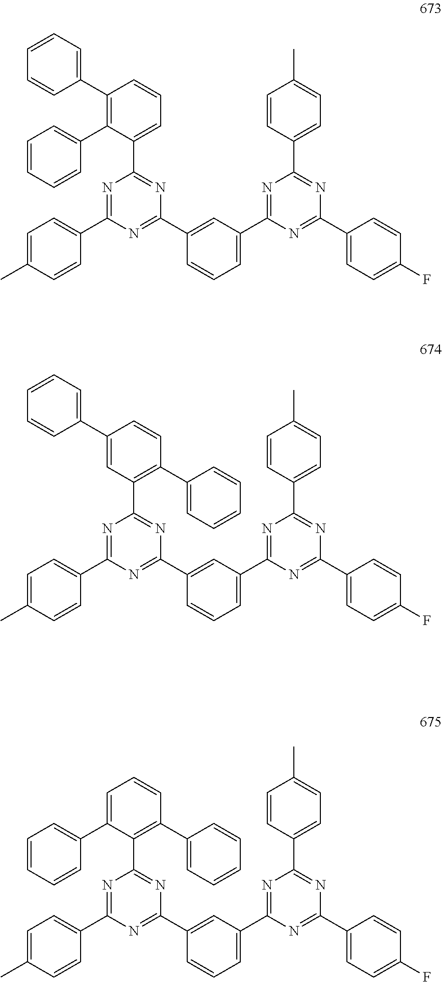 [SMILES: Cc1ccc(-c2nc(-c3ccc(F)cc3)nc(-c3cccc(-c4nc(-c5ccc(C)cc5)nc(-c5cccc(-c6ccccc6)c5-c5ccccc5)n4)c3)n2)cc1 ; Cc1ccc(-c2nc(-c3ccc(F)cc3)nc(-c3cccc(-c4nc(-c5ccc(C)cc5)nc(-c5cc(-c6ccccc6)ccc5-c5ccccc5)n4)c3)n2)cc1 ; Cc1ccc(-c2nc(-c3ccc(F)cc3)nc(-c3cccc(-c4nc(-c5ccc(C)cc5)nc(-c5c(-c6ccccc6)cccc5-c5ccccc5)n4)c3)n2)cc1]

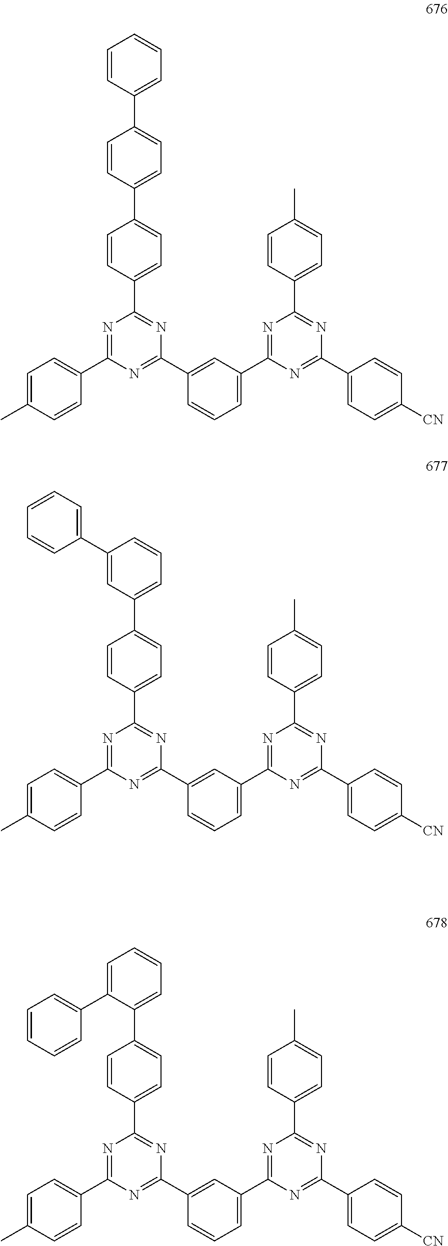 [SMILES: Cc1ccc(-c2nc(-c3ccc(C#N)cc3)nc(-c3cccc(-c4nc(-c5ccc(C)cc5)nc(-c5ccc(-c6ccc(-c7ccccc7)cc6)cc5)n4)c3)n2)cc1 ; Cc1ccc(-c2nc(-c3ccc(C#N)cc3)nc(-c3cccc(-c4nc(-c5ccc(C)cc5)nc(-c5ccc(-c6cccc(-c7ccccc7)c6)cc5)n4)c3)n2)cc1 ; Cc1ccc(-c2nc(-c3ccc(C#N)cc3)nc(-c3cccc(-c4nc(-c5ccc(C)cc5)nc(-c5ccc(-c6ccccc6-c6ccccc6)cc5)n4)c3)n2)cc1]

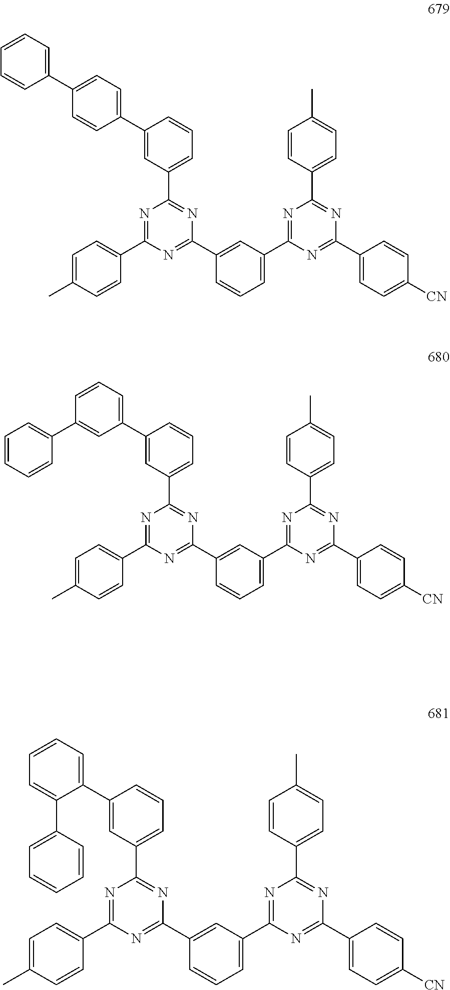 [SMILES: Cc1ccc(-c2nc(-c3ccc(C#N)cc3)nc(-c3cccc(-c4nc(-c5ccc(C)cc5)nc(-c5cccc(-c6ccc(-c7ccccc7)cc6)c5)n4)c3)n2)cc1 ; Cc1ccc(-c2nc(-c3ccc(C#N)cc3)nc(-c3cccc(-c4nc(-c5ccc(C)cc5)nc(-c5cccc(-c6cccc(-c7ccccc7)c6)c5)n4)c3)n2)cc1 ; Cc1ccc(-c2nc(-c3ccc(C#N)cc3)nc(-c3cccc(-c4nc(-c5ccc(C)cc5)nc(-c5cccc(-c6ccccc6-c6ccccc6)c5)n4)c3)n2)cc1]

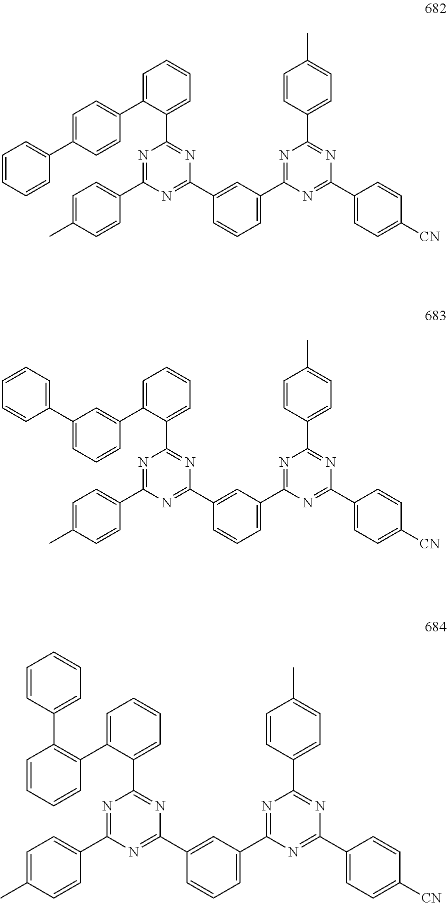 [SMILES: Cc1ccc(-c2nc(-c3ccc(C#N)cc3)nc(-c3cccc(-c4nc(-c5ccc(C)cc5)nc(-c5ccccc5-c5ccc(-c6ccccc6)cc5)n4)c3)n2)cc1 ; Cc1ccc(-c2nc(-c3ccc(C#N)cc3)nc(-c3cccc(-c4nc(-c5ccc(C)cc5)nc(-c5ccccc5-c5cccc(-c6ccccc6)c5)n4)c3)n2)cc1 ; Cc1ccc(-c2nc(-c3ccc(C#N)cc3)nc(-c3cccc(-c4nc(-c5ccc(C)cc5)nc(-c5ccccc5-c5ccccc5-c5ccccc5)n4)c3)n2)cc1]

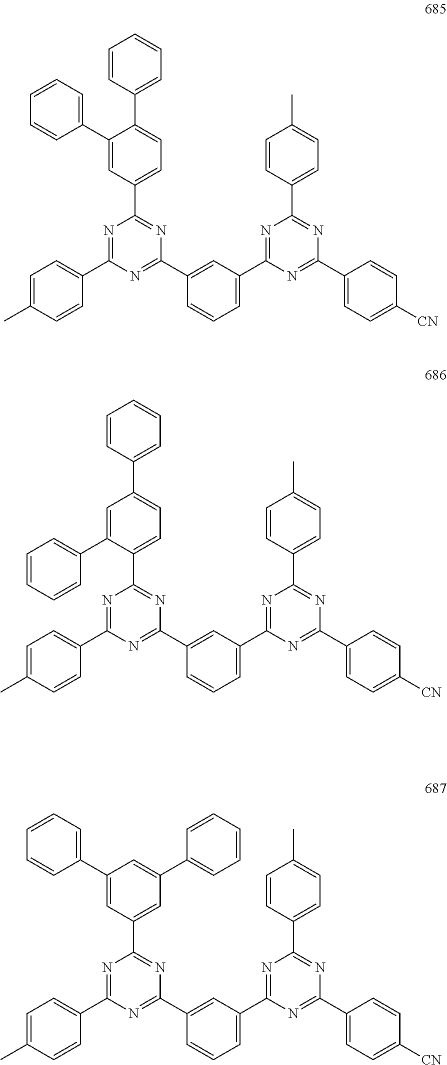 [SMILES: Cc1ccc(-c2nc(-c3ccc(C#N)cc3)nc(-c3cccc(-c4nc(-c5ccc(C)cc5)nc(-c5ccc(-c6ccccc6)c(-c6ccccc6)c5)n4)c3)n2)cc1 ; Cc1ccc(-c2nc(-c3ccc(C#N)cc3)nc(-c3cccc(-c4nc(-c5ccc(C)cc5)nc(-c5ccc(-c6ccccc6)cc5-c5ccccc5)n4)c3)n2)cc1 ; Cc1ccc(-c2nc(-c3ccc(C#N)cc3)nc(-c3cccc(-c4nc(-c5ccc(C)cc5)nc(-c5cc(-c6ccccc6)cc(-c6ccccc6)c5)n4)c3)n2)cc1]

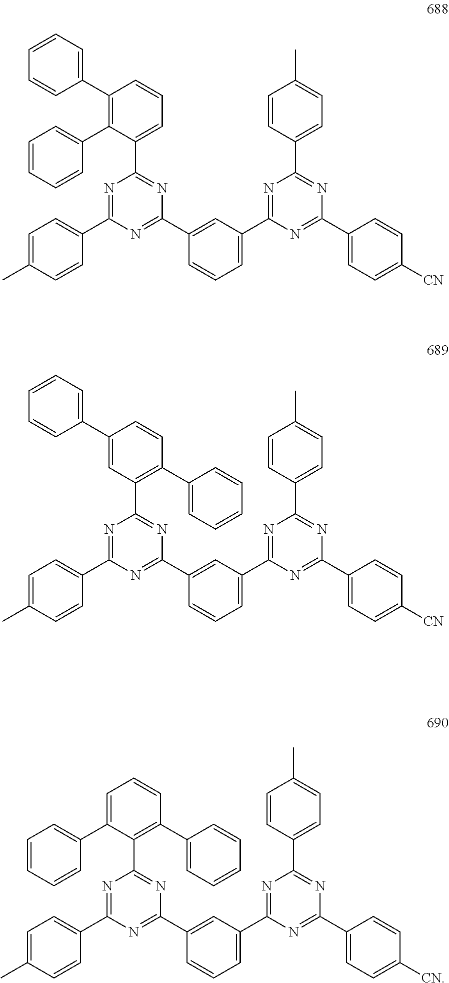 [SMILES: Cc1ccc(-c2nc(-c3ccc(C#N)cc3)nc(-c3cccc(-c4nc(-c5ccc(C)cc5)nc(-c5cccc(-c6ccccc6)c5-c5ccccc5)n4)c3)n2)cc1 ; Cc1ccc(-c2nc(-c3ccc(C#N)cc3)nc(-c3cccc(-c4nc(-c5ccc(C)cc5)nc(-c5cc(-c6ccccc6)ccc5-c5ccccc5)n4)c3)n2)cc1 ; Cc1ccc(-c2nc(-c3ccc(C#N)cc3)nc(-c3cccc(-c4nc(-c5ccc(C)cc5)nc(-c5c(-c6ccccc6)cccc5-c5ccccc5)n4)c3)n2)cc1]

9. A light-emitting device comprising:
a first electrode;
a second electrode facing the first electrode; and
an interlayer comprising an emission layer and between the first electrode and the second electrode,
wherein the interlayer comprises the compound represented by Formula 1 of claim 1.

10. The light-emitting device of claim 9, wherein:
the first electrode is an anode,
the second electrode is a cathode, and
the interlayer further comprises: a hole transport region between the first electrode and the emission layer and comprising a hole injection layer, a hole transport layer, an electron-blocking layer, an emission auxiliary layer, or any combination thereof; and/or an electron transport region between the second electrode and the emission layer and comprising a hole-blocking layer, an electron transport layer, an electron injection layer, or any combination thereof.

11. The light-emitting device of claim 9, wherein:
the electron transport region comprises the compound represented by Formula 1.

12. The light-emitting device of claim 9, wherein:
the interlayer comprises an electron transport layer, and the electron transport layer comprises the compound represented by Formula 1.

13. The light-emitting device of claim 9, wherein:
the emission layer comprises a first host, a second host, a first dopant, and a second dopant,
the first dopant is a compound comprising a ligand comprising a metal and an imidazole moiety, and
the second dopant is a compound comprising boron.

14. The light-emitting device of claim 13, wherein:
the metal comprises a transition metal.

15. The light-emitting device of claim 13, wherein:
the first host is a hole-transporting host.

16. The light-emitting device of claim 13, wherein:
the second host is an electron-transporting host.

17. The light-emitting device of claim 9, wherein:
the emission layer emits blue light.

18. An electronic apparatus comprising the light-emitting device of claim 9.

19. The electronic apparatus of claim 18, further comprising a thin-film transistor,
wherein the thin-film transistor includes a source electrode and a drain electrode, and
the first electrode of the light-emitting device is electrically connected to at least one selected from among the source electrode and the drain electrode of the thin-film transistor.

20. The electronic apparatus of claim 18, wherein:
the electronic apparatus comprises a display, a light source, lighting, a personal computer, a mobile phone, a digital camera, an electronic notebook, an electronic dictionary, an electronic game device, a medical device, a fish finder, a measuring device, a meter, or a projector.
